(19) United States
(12) Patent Application Publication
(10) Pub. No.: US 2026/0085062 A1
(43) Pub. Date: Mar. 26, 2026

(54) NOVEL COMPOUNDS AS CK2 INHIBITORS

(71) Applicant: Cambridge Enterprise Limited, Cambridge (GB)

(72) Inventors: Paul Glossop, Cambridgeshire (GB); Marko Juhana Hyvonen, Cambridge (GB); Paul Brear, Cambridgeshire (GB); David Robert Spring, Cambridgeshire (GB)

(21) Appl. No.: 19/109,724

(22) PCT Filed: Sep. 8, 2023
(86) PCT No.: PCT/GB23/52339

§ 371 Date: Mar. 7, 2025

(30) Foreign Application Priority Data
Sep. 8, 2022 (GB) 2213163.5

(51) Int. Cl. C07D 403/14; A61K 31/416; A61K 31/4196; A61K 31/422; A61K 31/4245; A61K 31/433; A61K 31/4439; A61K 31/444; A61K 31/501; C07D 231/56; C07D 401/04; C07D 401/14; C07D 403/04; C07D 403/12; C07D 405/04; C07D 413/04; C07D 413/14; C07D 417/04; C07D 471/04

(52) U.S. Cl. CPC C07D 403/14; A61K 31/416; A61K 31/4196; A61K 31/422; A61K 31/4245; A61K 31/433; A61K 31/4439; A61K 31/444; A61K 31/501; C07D 231/56; C07D 401/04; C07D 401/14; C07D 403/04; C07D 403/12; C07D 405/04; C07D 413/04; C07D 413/14; C07D 417/04; C07D 471/04

(57) ABSTRACT

Provided are compounds of the Formula I, and salts, hydrates and solvates thereof:

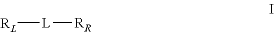

wherein R_{L}, L and R_{R}, and any groups associated therewith, are each as defined in the specification. The compounds are inhibitors of Casein Kinase 2 alpha (CK2α) and are useful for the treatment and/or prevention of diseases and conditions in which CK2α activity is implicated, such as, for example, but not limited to, the treatment and/or prevention of proliferative disorders (e.g. cancer), viral infections, inflammation, diabetes, vascular and ischemic disorders, neurodegeneration and the regulation of circadian rhythm. The present invention also relates to pharmaceutical compositions comprising the compounds defined herein and to their use for the treatment of diseases and/or conditions in which CK2α activity is implicated.

### INTRODUCTION

The present invention relates to novel therapeutic compounds. More specifically, the present invention relates to novel therapeutic compounds that inhibit Casein Kinase 2 alpha subunit (CK2α (CSNK2A1) and/or CK2α′ (CSNK2A2)) and as part of the CK2 holoenzyme. The novel therapeutic compounds are therefore useful for the treatment and/or prevention of diseases and conditions in which CK2α activity is implicated, such as, for example but not limited to, the treatment and/or prevention of proliferative disorders (e.g. cancer), viral infections, inflammation, diabetes, vascular and ischemic disorders, neurodegeneration and the regulation of circadian rhythm.

The present invention also relates to pharmaceutical compositions comprising the novel therapeutic compounds defined herein, to processes for synthesising these compounds and to their use for the treatment of diseases and/or conditions in which CK2α activity is implicated.

### BACKGROUND OF THE INVENTION

CK2α is a serine/threonine kinase that is a key regulator of many cellular processes and is involved in cellular proliferation and anti-apoptotic mechanisms (Battistutta & Lolli, Mol. Cell. Biochem. 2011). It mainly exists as a holoenzyme composed of two catalytic (α and/or α′) and a dimer of regulatory (p) subunits, but it can also be found as the isolated subunits (Niefind et al, EMBO J 2001). Unlike most other kinases, it is constitutively active and more than 300 proteins have been identified as putative CK2α substrates, making it one of the most pleiotropic proteins in eukaryotic systems (Meggio & Pinna, FASEB 2003).

CK2α is a pro-survival kinase that operates across multiple signaling pathways to convey a proliferative and anti-apoptotic phenotype to cells. Consequently, cancer cells are often described as being addicted to CK2α activity and a high-profile genome-wide CRISPR-Cas9 screen highlighted CK2α as a top tier, high priority drug target for Colorectal Cancer (CRC) (Behan et al, Nature 2019). The target is well validated by human data that correlates poor patient survival in numerous tumor types, including CRC, with increased CK2α expression (Lin et al, PLoS ONE 2011). Additionally, data from clinical samples shows CK2α expression is upregulated in numerous tumor types (Ortega et al, PLoS ONE 2014; Di Maira et al, 2019).

The human genetics of CRC are well characterized and approximately 80% tumors are identified as being wnt pathway mutation driven (e.g. APC, β-catenin) (Zhan et al, Oncogene 2017). The wnt pathway is known to be sensitive to and amplified by CK2α activity and can be inhibited by loss of CK2α function (Gao & Wang, J B C 2006). For example, in animal models, CK2α inhibition prevents tumor growth that is driven by different mutations in the wnt pathway (Dowling et al, ACS 2016).

CK2α also contributes to the malignant phenotype in cholangiocarcinoma (CCA), which is known to be a wnt-dysregulated tumor type (Zhan et al, Oncogene 2017). CK2α is over-expressed in human CCA samples and CCA tumor cell lines (Di Maira et al, Oncogenesis 2019); and disruption of CK2α activity in CCA cell models is reported to inhibit tumorigenic properties (Zakharia et al, Translational Oncology 2019).

It is hypothesised that a CK2α inhibitor given either as a monotherapy, in combination with standard of care chemotherapy or in combination with other targeted therapies in development, such as, but not limited to, KRAS inhibitors, will inhibit CRC tumor growth by reversing aberrant upregulation of wnt signaling to restore the normal balance of apoptosis and proliferation.

Existing CK2α inhibitors target the highly conserved ATP binding site. This design strategy often leads to a poor selectivity profile for such inhibitors over other kinases. There is therefore a need for potent and more selective CK2α inhibitors that bind to the catalytic ATP site of CK2α (to drive potent enzyme inhibition) but also interact with other areas of CK2α, such as the αD site (to drive high levels of selectivity over other kinases).

The present invention was devised with the foregoing in mind.

### SUMMARY OF THE INVENTION

In one aspect, the present invention provides a compound of Formula I as defined herein, and/or a pharmaceutically acceptable salt, hydrate or solvate thereof.

In another aspect, the present invention provides a pharmaceutical composition which comprises a compound of Formula I as defined herein, or a pharmaceutically acceptable salt, hydrate or solvate thereof, and one or more pharmaceutically acceptable excipients.

In another aspect, the present invention provides a compound of Formula I as defined herein, or a pharmaceutically acceptable salt, hydrate or solvate thereof, or a pharmaceutical composition as defined herein, for use in therapy.

In another aspect, the present invention provides a compound of Formula I as defined herein, or a pharmaceutically acceptable salt, hydrate or solvate thereof, or a pharmaceutical composition as defined herein, for use in the treatment of a disease or condition in which CK2α activity is implicated.

In another aspect, the present invention provides a compound of Formula I as defined herein, or a pharmaceutically acceptable salt, hydrate or solvate thereof, or a pharmaceutical composition as defined herein, for use in the treatment of a disease or condition associated with aberrant activity of CK2α.

In another aspect, the present invention provides a compound of Formula I as defined herein, or a pharmaceutically acceptable salt, hydrate or solvate thereof, or a pharmaceutical composition as defined herein, for use in the treatment of proliferative disorders (e.g. cancer or benign neoplasms), viral infections, an inflammatory disease or condition, diabetes, vascular and ischemic disorders, neurodegenerative disorders and/or the regulation of circadian rhythm.

In another aspect, the present invention provides a compound of Formula I as defined herein, or a pharmaceutically acceptable salt, hydrate or solvate thereof, or a pharmaceutical composition as defined herein, for use in the treatment of a cancer.

In another aspect, the present invention provides a compound of Formula I as defined herein, or a pharmaceutically acceptable salt, hydrate or solvate thereof, or a pharmaceutical composition as defined herein, for use in the treatment of a viral infection.

In another aspect, the present invention provides the use of a compound of Formula I as defined herein, or a pharmaceutically acceptable salt, hydrate or solvate thereof, in the manufacture of a medicament for use in the treatment of a disease or condition in which CK2α activity is implicated.

In another aspect, the present invention provides the use of a compound of Formula I as defined herein, or a pharmaceutically acceptable salt, hydrate or solvate thereof, in the manufacture of a medicament for use in the treatment of a disease or condition associated with aberrant activity of CK2α.

In another aspect, the present invention provides the use of a compound of Formula I as defined herein, or a pharmaceutically acceptable salt, hydrate or solvate thereof, in the manufacture of a medicament for use in the treatment of proliferative disorders (e.g. cancer or benign neoplasms), viral infections, an inflammatory disease or condition, diabetes, vascular and ischemic disorders, neurodegenerative disorders and/or the regulation of circadian rhythm.

In another aspect, the present invention provides the use of a compound of Formula I as defined herein, or a pharmaceutically acceptable salt, hydrate or solvate thereof, in the manufacture of a medicament for use in the treatment of a cancer.

In another aspect, the present invention provides the use of a compound of Formula I as defined herein, or a pharmaceutically acceptable salt, hydrate or solvate thereof, in the manufacture of a medicament for use in the treatment of a viral infection.

In another aspect, the present invention provides a method of treating a disease or condition in which CK2α activity is implicated, said method comprising administering to a subject in need thereof an effective amount of a compound of Formula I as defined herein, or a pharmaceutically acceptable salt, hydrate or solvate thereof, or a pharmaceutical composition as defined herein.

In another aspect, the present invention provides a method of treating a disease or condition associated with aberrant activity of CK2α, said method comprising administering to a subject in need thereof an effective amount of a compound of Formula I as defined herein, or a pharmaceutically acceptable salt, hydrate or solvate thereof, or a pharmaceutical composition as defined herein.

In another aspect, the present invention provides a method of treating a proliferative disorder (e.g. cancer or benign neoplasms), a viral infection, an inflammatory disease or condition, diabetes, vascular and ischemic disorders, neurodegenerative disorders and/or regulating cardiac rhythm, said method comprising administering to a subject in need thereof an effective amount of a compound of Formula I as defined herein, or a pharmaceutically acceptable salt, hydrate or solvate thereof, or a pharmaceutical composition as defined herein.

In another aspect, the present invention provides a method of treating cancer, said method comprising administering to a subject in need thereof an effective amount of a compound of Formula I as defined herein, or a pharmaceutically acceptable salt, hydrate or solvate thereof, or a pharmaceutical composition as defined herein.

In another aspect, the present invention provides a method of treating a viral infection, said method comprising administering to a subject in need thereof an effective amount of a compound of Formula I as defined herein, or a pharmaceutically acceptable salt, hydrate or solvate thereof, or a pharmaceutical composition as defined herein.

In another aspect, the present invention provides a combination treatment comprising a compound of Formula I, or a pharmaceutically acceptable salt, hydrate or solvate thereof, as defined herein, with one or more additional therapeutic agents.

In another aspect, the present invention provides processes for preparing compounds of Formula I as defined herein, or a pharmaceutically acceptable salt, hydrate or solvate thereof, as defined herein, with one or more additional therapeutic agents.

Preferred, suitable, and optional features of any one particular aspect of the present invention are also preferred, suitable, and optional features of any other aspect.

### DETAILED DESCRIPTION OF THE INVENTION

### Definitions

Unless otherwise stated, the following terms used in the specification and claims have the following meanings set out below.

It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

References to “Casein Kinase 2 alpha” or “CK2α” herein include CK2α (CSNK2A1) and/or CK2α′ (CSNK2A2). Where reference is made to the compounds of the present invention defined herein inhibiting CK2α or being CK2α inhibitors, we mean that the compounds function as inhibitors of CK2α (CSNK2A1) and/or CK2α′ (CSNK2A2) and the CK2 holoenzyme. In a particular embodiment, the compounds of the invention inhibit CK2α (CSNK2A1). In another embodiment, the compounds of the invention inhibit CK2α′ (CSNK2A2).

The compounds and intermediates described herein may be named according to either the IUPAC (International Union for Pure and Applied Chemistry) or CAS (Chemical Abstracts Service) nomenclature systems. It should be understood that unless expressly stated to the contrary, the terms “compounds of Formula I”, “compounds of the invention” and the more general term “compounds” refer to and include any and all compounds described by and/or with reference to Formula I herein. It should also be understood that these terms encompass all stereoisomers, i.e. cis and trans isomers, as well as optical isomers, i.e. R and S enantiomers, of such compounds, in substantially pure form and/or any mixtures of the foregoing in any ratio. This understanding extends to pharmaceutical compositions and methods of treatment that employ or comprise one or more compounds of the Formula I, either by themselves or in combination with additional agents.

Unless specified otherwise, atoms are referred to herein by their chemical symbol as appearing in the IUPAC periodic table of the Elements. For example, “C” refers to a carbon atom.

The term “(m-nC)” or “(m-nC) group” used alone or as a prefix, refers to any group having m to n carbon atoms.

In this specification the term “alkyl” includes both straight and branched chain alkyl groups. References to individual alkyl groups such as “propyl” are specific for the straight chain version only and references to individual branched chain alkyl groups such as “isopropyl” are specific for the branched chain version only. For Example, “(1-6C)alkyl” includes (1-4C)alkyl, (1-3C)alkyl, propyl, isopropyl and t-butyl. A similar convention applies to other radicals, for example “phenyl(1-6C)alkyl” includes phenyl(1-4C)alkyl, benzyl, 1-phenylethyl and 2-phenylethyl.

An “alkylene” group is an alkyl group that is positioned between and serves to connect two other chemical groups. Thus, “(1-6C)alkylene” means a linear saturated divalent hydrocarbon radical of one to six carbon atoms or a branched saturated divalent hydrocarbon radical of three to six carbon atoms, for example, methylene, ethylene, propylene, 2-methylpropylene, pentylene, and the like.

“(3-6C)cycloalkyl” means a hydrocarbon ring containing from 3 to 6 carbon atoms, for example, cyclopropyl, cyclobutyl, cyclopentyl or cyclohexyl.

“(3-6C)cycloalkoxy” refers to cycloalkoxy groups (i.e. O-cycloalkyl group) wherein the cycloalkyl group means a hydrocarbon ring containing from 3 to 6 carbon atoms, for example, —O-cyclopropyl, —O-cyclobutyl, —O-cyclopentyl or —O-cyclohexyl.

The term “halo”, “halogen” or “halogeno” refers to fluoro, chloro, bromo and iodo.

As used herein by themselves or in conjunction with another term or terms, “haloalkyl” and “haloalkyl group” refer to alkyl groups in which one or more hydrogen atoms are replaced by halogen atoms. Representative examples include, but are not limited to, —CF₃, —CHF₂, —CH₂F, —CF₂CF₃, —CHFCF₃, and —CH₂CF₃. Suitably, a haloalkyl group is selected from —CHF₂ and —CF₃, suitably —CF₃.

As used herein by themselves or in conjunction with another term or terms, “haloalkoxy” and “haloalkoxy group” refer to alkoxy groups (i.e. O-alkyl groups) in which one or more hydrogen atoms are replaced by halogen atoms. Representative examples include, but are not limited to, —OCF₃, —OCHF₂, —OCH₂F, and —OCF₂CF₃. Suitably, a haloalkoxy group is selected from —OCHF₂ and —OCF₃, suitably —OCF₃.

The term “heterocyclyl”, “heterocyclic” or “heterocycle” means a non-aromatic saturated or partially saturated monocyclic, fused, bridged, or spiro bicyclic heterocyclic ring system(s). Monocyclic heterocyclic rings contain from about 3 to 12 (suitably from 3 to 7) ring atoms, with from 1 to 5 (suitably 1, 2 or 3) heteroatoms selected from nitrogen, oxygen or sulfur in the ring. Bicyclic heterocycles contain from 7 to 17 member atoms, suitably 7 to 12 member atoms, in the ring. Bicyclic heterocyclic(s) rings may be fused, spiro, or bridged ring systems. Examples of heterocyclic groups include cyclic ethers such as, but not limited to, oxiranyl, oxetanyl, tetrahydrofuranyl, dioxanyl, and substituted cyclic ethers. Heterocycles containing nitrogen include, for example, azetidinyl, pyrrolidinyl, piperidinyl, piperazinyl, tetrahydrotriazinyl, tetrahydropyrazolyl, and the like. Typical sulfur containing heterocycles include tetrahydrothienyl, dihydro-1,3-dithiol, tetrahydro-2H-thiopyran, and hexahydrothiepine. Other heterocycles include dihydrooxathiolyl, tetrahydrooxazolyl, tetrahydro-oxadiazolyl, tetrahydrodioxazolyl, tetrahydrooxathiazolyl, hexahydrotriazinyl, tetrahydrooxazinyl, morpholinyl, thiomorpholinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothienyl and thiomorpholinyl such as, but not limited to, tetrahydrothiene 1,1-dioxide and thiomorpholinyl 1,1-dioxide. A suitable value for a heterocyclyl group which bears 1 or 2 oxo (═O) or thioxo (=S) substituents is, for example, 2-oxopyrrolidinyl, 2-thioxopyrrolidinyl, 2-oxoimidazolidinyl, 2-thioxoimidazolidinyl, 2-oxopiperidinyl, 2,5-dioxopyrrolidinyl, 2,5-dioxoimidazolidinyl or 2,6-dioxopiperidinyl. Particular heterocyclyl groups are saturated monocyclic 3 to 7 membered heterocyclyls containing 1, 2 or 3 heteroatoms selected from nitrogen, oxygen or sulfur, for example azetidinyl, tetrahydrofuranyl, tetrahydropyranyl, pyrrolidinyl, morpholinyl, tetrahydrothienyl, tetrahydrothienyl 1,1-dioxide, thiomorpholinyl, thiomorpholinyl 1,1-dioxide, piperidinyl, homopiperidinyl, piperazinyl or homopiperazinyl. As the skilled person would appreciate, any heterocycle may be linked to a group via any suitable atom, such as via a carbon or nitrogen atom. However, reference herein to piperidino or morpholino refers to a piperidin-1-yl or morpholin-4-yl ring that is linked via the ring nitrogen.

By “bridged ring systems” is meant ring systems in which two rings share more than two atoms, see for example Advanced Organic Chemistry, by Jerry March, 4^{th} Edition, Wiley Interscience, pages 131-133, 1992. Examples of bridged heterocyclyl ring systems include, aza-bicyclo[2.2.1]heptane, 2-oxa-5-azabicyclo[2.2.1]heptane, aza-bicyclo[2.2.2]octane, aza-bicyclo[3.2.1]octane and quinuclidine.

By “spiro bicyclic ring systems” we mean that the two ring systems share one common spiro carbon atom, i.e. the heterocyclic ring is linked to a further carbocyclic or heterocyclic ring through a single common spiro carbon atom. Examples of spiro ring systems include 6-azaspiro[3.4]octane, 2-oxa-6-azaspiro[3.4]octane, 2-azaspiro[3.3]heptanes, 2-oxa-6-azaspiro[3.3]heptanes, 7-oxa-2-azaspiro[3.5]nonane, 6-oxa-2-azaspiro[3.4]octane, 2-oxa-7-azaspiro[3.5]nonane and 2-oxa-6-azaspiro[3.5]nonane.

The term “heteroaryl” or “heteroaromatic” means an aromatic mono-, bi-, or polycyclic ring incorporating one or more (for example 14, particularly 1, 2 or 3) heteroatoms selected from nitrogen, oxygen or sulfur. The term heteroaryl includes both monovalent species and divalent species. Examples of heteroaryl groups are monocyclic and bicyclic groups containing from five to twelve ring members, and more usually from five to ten ring members.

The heteroaryl group can be, for example, a 5- or 6-membered monocyclic ring or a 9- or 10-membered bicyclic ring, for example a bicyclic structure formed from fused five and six membered rings or two fused six membered rings. Each ring may contain up to about four heteroatoms typically selected from nitrogen, sulfur and oxygen. Typically, the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general, the number of basic nitrogen atoms present in the heteroaryl group, including any amino group substituents of the ring, will be less than five. The nitrogen atom of a heteroaryl may occasionally be in the form of a N-oxide (N⁺—O⁻).

Examples of heteroaryl include furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, isoindolyl, benzothienyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl, pteridinyl, naphthyridinyl, carbazolyl, phenazinyl, benzisoquinolinyl, pyridopyrazinyl, thieno[2,3b]-furanyl-, 2H-furo[3,2b]-pyranyl-, 5H-pyrido[2,3-d]-ooxazinyl-, 1H-pyrazolo[4,3-d]-oxazolyl, 4H-imidazo[4,5d]thiazolyl, pyrazino[2,3d]pyridazinyl, -imidazo[2,1b]thiazolyl, -imidazo[1,2b][1,2,4]-triazinyl. The term heteroaryl also covers exemplified groups such as those depicted below:

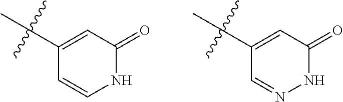 [SMILES: *c1cc[nH]c(=O)c1 ; *c1cn[nH]c(=O)c1]

as these groups tautomerise to the following:

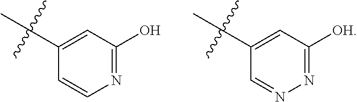 [SMILES: *c1ccnc(O)c1 ; *c1cnnc(O)c1]

Occasionally, a N atom present in a heteroaryl ring may be present N⁺—O⁻.

“Heteroaryl” also covers partially aromatic bi- or polycyclic ring systems wherein at least one ring is an aromatic ring and one or more of the other ring(s) is a nonaromatic, saturated or partially saturated ring, provided at least one ring contains one or more heteroatoms selected from nitrogen, oxygen or -sulfur-. Examples of partially aromatic heteroaryl groups include for example, tetrahydroisoquinolinyl, tetrahydroquinolinyl, 2-oxo-1,2,3,4-tetrahydroquinolinyl, dihydrobenzthienyl, dihydrobenzfuranyl, 2,3-dihydro-benzo[1,4]dioxinyl, benzo[1,3]dioxolyl, 2,2-dioxo-1,3-dihydro-2-benzothienyl, 4,5,6,7-tetrahydrobenzofuranyl, indolinyl, 1,2,3,4-tetrahydro-1,8-naphthyridinyl, 1,2,3,4-tetrahydropyrido[2,3-b]pyrazinyl, 3,4-dihydro-2H-pyrido[3,2-b][1,4]oxazinyl and 6,8-dihydro-5H-[1,2,4]triazolo[4,3-a]pyrazinyl.

Examples of five membered heteroaryl groups include but are not limited to pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl and tetrazolyl groups.

Examples of six membered heteroaryl groups include but are not limited to pyridyl, pyrazinyl, pyridazinyl, pyrimidinyl and triazinyl.

A bicyclic heteroaryl group may be, for example, a group selected from:
a benzene ring fused to a 5- or 6-membered ring containing 1, 2 or 3 ring heteroatoms;
a pyridine ring fused to a 5- or 6-membered ring containing 1, 2 or 3 ring heteroatoms;
a pyrimidine ring fused to a 5- or 6-membered ring containing 1 or 2 ring heteroatoms;
a pyrrole ring fused to a 5- or 6-membered ring containing 1, 2 or 3 ring heteroatoms;
a pyrazole ring fused to a 5- or 6-membered ring containing 1 or 2 ring heteroatoms;
a pyrazine ring fused to a 5- or 6-membered ring containing 1 or 2 ring heteroatoms;
an imidazole ring fused to a 5- or 6-membered ring containing 1 or 2 ring heteroatoms;
an oxazole ring fused to a 5- or 6-membered ring containing 1 or 2 ring heteroatoms;
an isoxazole ring fused to a 5- or 6-membered ring containing 1 or 2 ring heteroatoms;
a thiazole ring fused to a 5- or 6-membered ring containing 1 or 2 ring heteroatoms;
an isothiazole ring fused to a 5- or 6-membered ring containing 1 or 2 ring heteroatoms;
a thiophene ring fused to a 5- or 6-membered ring containing 1, 2 or 3 ring heteroatoms;
a furan ring fused to a 5- or 6-membered ring containing 1, 2 or 3 ring heteroatoms;
a cyclohexyl ring fused to a 5- or 6-membered heteroaromatic ring containing 1, 2 or 3 ring heteroatoms; and
a cyclopentyl ring fused to a 5- or 6-membered heteroaromatic ring containing 1, 2 or 3 ring heteroatoms.

Particular examples of bicyclic heteroaryl groups containing a six membered ring fused to a five membered ring include but are not limited to benzfuranyl, benzthiophenyl, benzimidazolyl, benzoxazolyl, benzisoxazolyl, benzthiazolyl, benzisothiazolyl, isobenzofuranyl, indolyl, isoindolyl, indolizinyl, indolinyl, isoindolinyl, purinyl (e.g., adeninyl, guaninyl), indazolyl, benzodioxolyl and pyrazolopyridinyl groups.

Particular examples of bicyclic heteroaryl groups containing two fused six membered rings include but are not limited to quinolinyl, isoquinolinyl, chromanyl, thiochromanyl, chromenyl, isochromenyl, chromanyl, isochromanyl, benzodioxanyl, quinolizinyl, benzoxazinyl, benzodiazinyl, pyridopyridinyl, quinoxalinyl, quinazolinyl, cinnolinyl, phthalazinyl, naphthyridinyl and pteridinyl groups.

The term “aryl” means a cyclic or polycyclic aromatic ring having from 5 to 12 carbon atoms. The term aryl includes both monovalent species and divalent species. Examples of aryl groups include, but are not limited to, phenyl, biphenyl, naphthyl and the like. In particular embodiment, an aryl is phenyl.

This specification also makes use of several composite terms to describe groups comprising more than one functionality. Such terms will be understood by a person skilled in the art. For Example, heterocyclyl(m-nC)alkyl comprises (m-nC)alkyl substituted by heterocyclyl.

The term “aryl(1-2C)alkyl” means an aryl group covalently attached to a (1-2C)alkylene group, both of which are defined herein. Examples of aryl-(1-2C)alkyl groups include benzyl, phenylethyl, and the like.

“Heteroaryl(1-3C)alkyl” means a heteroaryl group covalently attached to a (1-3C)alkylene group, both of which are defined herein. Examples of heteroaryl-alkyl groups include pyridin-3-ylmethyl, 2-(benzofuran-2-yl)ethyl, and the like.

“Heterocyclyl(1-2C)alkyl” means a heterocyclyl group covalently attached to a (1-2C)alkylene group, both of which are defined herein.

“(3-6C)cycloalkyl-(1-2C)alkyl” means a (3-6C)cycloalkyl group covalently attached to a (1-2C)alkylene group, both of which are defined herein.

The term “optionally substituted” refers to either groups, structures, or molecules that are substituted and those that are not substituted. The term “wherein a/any CH, CH₂, CH₃ group or heteroatom (i.e. NH) within a R¹ group is optionally substituted” suitably means that (any) one of the hydrogen radicals of the R¹ group is substituted by a relevant stipulated group.

Where optional substituents are chosen from “one or more” groups it is to be understood that this definition includes all substituents being chosen from one of the specified groups or the substituents being chosen from two or more of the specified groups.

A wavy bond ( [symbol] ) is used herein to show a point of attachment.

The phrase “compound of the invention” means those compounds which are disclosed herein, both generically and specifically.

As used herein by itself or in conjunction with another term or terms, “pharmaceutically acceptable” refers to materials that are generally chemically and/or physically compatible with other ingredients (such as, for example, with reference to a formulation), and/or are generally physiologically compatible with the recipient (such as, for example, a subject) thereof.

As used herein by themselves or in conjunction with another term or terms, “subject(s)” and “patient(s)”, suitably refer to mammals, in particular humans.

### Compounds of the Invention

In a first aspect, the present invention relates to a compound, or pharmaceutically acceptable salt, hydrate or solvate thereof, having the structural formula I shown below:

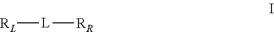

wherein:
L is linker that separates R_{L} from R_{R} by 7 to 13 bond lengths;
R_{L} is selected from one of formulae Ic, Id, Ie, If, Ig, Ih, Ii, Ij or Ik shown below:

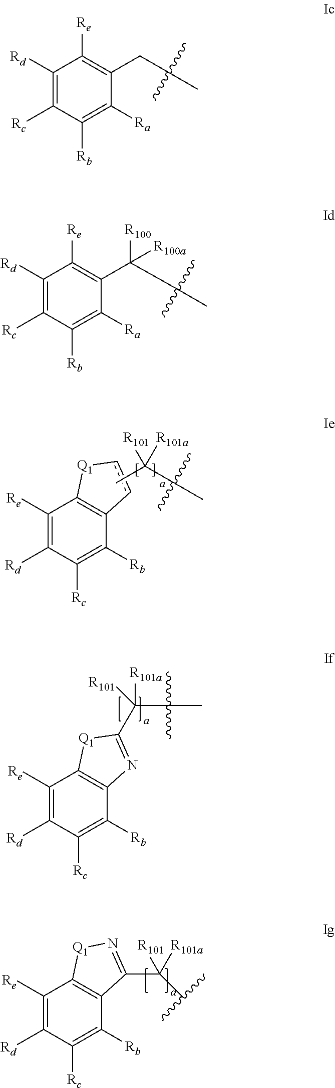

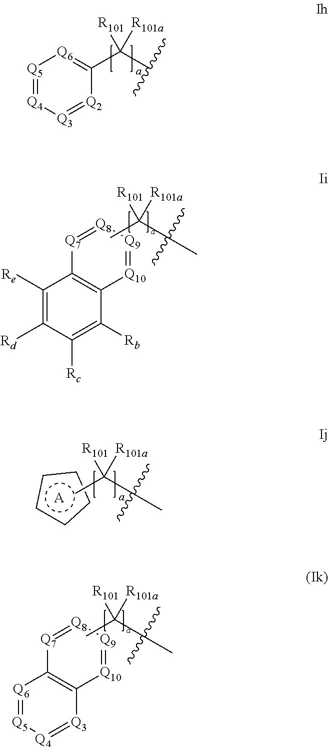

wherein:
Rₐ and Rₑ are each independently selected from hydrogen, methyl or halo;
R_{b} and R_{d} are each independently selected from hydrogen, halo, cyano, (1-4C)alkyl, (3-6C)cycloalkyl,
   —[CH₂]₀₋₃-(1-4C)alkoxy,
   —[CH₂]₀₋₃—C(O)NH₂,
   —[CH₂]₀₋₃—C(O)NH(1-4C)alkyl,
   —[CH₂]₀₋₃—C(O)N[(1-4C)alkyl]₂,
   —[CH₂]₀₋₃—NH₂,
   —[CH₂]₀₋₃—NH(1-4C)alkyl,
   —[CH₂]₀₋₃—N[(1-4C)alkyl]₂,
   —[CH₂]₀₋₃—S(O)_{q}-(1-4C)alkyl (wherein q is 0, 1 or 2),
   —[CH₂]₀₋₃—C(O)(1-4C)alkyl,
   —[CH₂]₀₋₃—C(O)OH,
   —[CH₂]₀₋₃—C(O)O-(1-4C)alkyl,
   —[CH₂]₀₋₃—N(R_{f})C(O)-(1-4C)alkyl (wherein R_{f} is hydrogen or methyl),
   —[CH₂]₀₋₃—S(O)₂NH(1-4C)alkyl,
   —[CH₂]₀₋₃—S(O)₂N[(1-4C)alkyl]₂,
   —[CH₂]₀₋₃—N(R₉)SO₂-(1-4C)alkyl (wherein R₉ is hydrogen or methyl),
   a group of the formula:

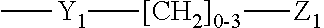

wherein Y₁ is absent, —O—, —NH—, —NMe-, —S—, —S(O)— or —S(O)₂—; and
Z₁ is (3-6C)cycloalkyl, phenyl, a 4- to 6-membered heterocyclyl or 5 or 6-membered heteroaryl;
and wherein:
any alkyl, alkoxy, cycloalkyl or —[CH₂]— moiety within a R_{b} and R_{d} substituent group is optionally substituted by one or more substituents selected from halo, hydroxy, cyano, amino, —C(O)OH, —C(O)NH₂, (1-2C)alkoxy, or (3-4C)cycloalkoxy; and
Z₁ is optionally substituted by one or more substituents selected from: halo, hydroxy, cyano, amino, —C(O)OH, —C(O)NH₂, (1-2C)alkoxy, (1-2C)alkyl, (3-4C)cycloalkyl, (3-4C)cycloalkoxy, —C(O)NH(1-2C)alkyl, —C(O)N[(1-2C)alkyl]₂, —NH(1-2C)alkyl, —N[(1-2C)alkyl]₂, —S(O)_{q}-(1-2C)alkyl (wherein q is 0, 1 or 2), —C(O)(1-2C)alkyl, —C(O)O-(1-2C)alkyl, —N(R_{f})C(O)-(1-2C)alkyl, —S(O)₂NH(1-2C)alkyl, —S(O)₂N[(1-2C)alkyl]₂, or —NHSO₂-(1-2C)alkyl, and wherein any (1-2C)alkoxy, (1-2C)alkyl, (3-4C)cycloalkyl or (3-4C)cycloalkoxy group is optionally substituted by one or more substituents selected from halo, cyano, hydroxy, (1-2C)alkyl, (1-2C)alkoxy or (1-2C)alkoxy-(1-2C)alkyl;
R_{c} is selected from hydrogen, halo, cyano, —C(O)NH₂, (1-4C)alkyl, (3-6C)cycloalkyl
   —[CH₂]₀₋₃-(1-4C)alkoxy,
   —[CH₂]₀₋₃-(3-6C)cycloalkoxy,
   —[CH₂]₀₋₃—C(O)NH₂,
   —[CH₂]₀₋₃—C(O)NH(1-4C)alkyl,
   —[CH₂]₀₋₃—C(O)N[(1-4C)alkyl]₂,
   —[CH₂]₀₋₃—NH₂,
   —[CH₂]₀₋₃—NH(1-4C)alkyl,
   —[CH₂]₀₋₃—N[(1-4C)alkyl]₂,
   —[CH₂]₀₋₃—S(O)_{q}-(1-4C)alkyl (wherein q is 0, 1 or 2),
   —[CH₂]₀₋₃—C(O)(1-4C)alkyl,
   —[CH₂]₀₋₃—C(O)OH,
   —[CH₂]₀₋₃—C(O)O-(1-4C)alkyl,
   —[CH₂]₀₋₃—N(Rn)C(O)-(1-4C)alkyl (wherein Rₕ is hydrogen or methyl),
   —[CH₂]₀₋₃—S(O)₂NH(1-4C)alkyl,
   —[CH₂]₀₋₃—S(O)₂N[(1-4C)alkyl]₂,
   —[CH₂]₀₋₃—N(Ri)SO₂-(1-4C)alkyl (wherein Ri is hydrogen or methyl),
   a group of the formula:

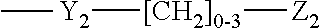

wherein Y₂ is absent, —O—, —NH—, —NMe-, —S—, —S(O)— or —S(O)₂—; and
Z₂ is (3-6C)cycloalkyl, phenyl, a 4- to 6-membered heterocyclyl or 5 or 6-membered heteroaryl;
and wherein:
   any alkyl, alkoxy, cycloalkyl or —[CH₂]— moiety within a R_{c} substituent group is optionally substituted by one or more substituents selected from halo, hydroxy, cyano, amino, —C(O)OH, —C(O)NH₂, (1-2C)alkoxy, or (3-4C)cycloalkoxy; and
   Z₂ is optionally substituted by one or more substituents selected from: halo, hydroxy, cyano, amino, —C(O)OH, —C(O)NH₂, (1-2C)alkoxy, (1-2C)alkyl, (3-4C)cycloalkyl, (3-4C)cycloalkoxy, —C(O)NH(1-2C)alkyl, —C(O)N[(1-2C)alkyl]₂, —NH(1-2C)alkyl, —N[(1-2C)alkyl]₂, —S(O)_{q}-(1-2C)alkyl (wherein q is 0, 1 or 2), —C(O)(1-2C)alkyl, —C(O)O-(1-2C)alkyl, —N(R_{f})C(O)-(1-2C)alkyl, —S(O)₂NH(1-2C)alkyl, —S(O)₂N[(1-2C)alkyl]₂, or —NHSO₂-(1-2C)alkyl, and wherein any (1-2C)alkoxy, (1-2C)alkyl, (3-4C)cycloalkyl or (3-4C)cycloalkoxy group is optionally substituted by one or more substituents selected from halo, cyano, hydroxy, (1-2C)alkyl, (1-2C)alkoxy or (1-2C)alkoxy-(1-2C)alkyl;
R₁₀₀ is (1-2C)alkyl optionally substituted by hydroxy or halo;
R₁₀₀ₐ is selected from hydrogen or methyl;
or R₁₀₀ and R₁₀₀ₐ are linked to form a cyclopropyl or cyclobutyl ring;
R₁₀₁ is hydrogen or (1-2C)alkyl optionally substituted by hydroxy or halo;
R₁₀₁ₐ is selected from hydrogen or methyl;
or R₁₀₁ and R₁₀₁ₐ are linked to form a cyclopropyl or cyclobutyl ring;
integer a is 0, 1 or 2;
Q₁ is selected from —NR₁₀₂—, —O—, —S— or —CH—;
R₁₀₂ is hydrogen or (1-2C)alkyl;
[symbol] represents a single or double bond;
Q₂ is N or CRₐ;
Q₃ is N or CR_{b};
Q₄ is N or CR_{c};
Q₅ is N or CR_{d};
Q₅ is N or CRₑ;
Rₐ, R_{b}, R_{c}, R_{d} and Re are each as defined above:
   with the proviso that one to three of Q₂, Q₃, Q₄, Q₅ or Q₆ is/are N;
Q₇ is N or CR_{f};
Q₈ is N or CR_{f};
Q₉ is N or CR_{f};
Q₁₀ is N or CR_{f};
   with the proviso that one or two of Q₇, Q₈, Q₉ or Q₁₀ is/are N;
each R_{f} present is independently selected from hydrogen, methyl or halo;
Ring A is a five-membered heteroaryl ring optionally substituted by one R_{b} and/or one or two R_{c} substituents;
R_{R} is selected from one of formulae Im, In or Io shown below:

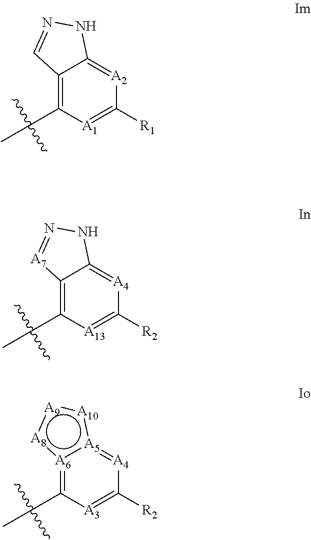

wherein:
[symbol] denotes the point of attachment;
A₁ and A₂ are both CH; or one of A₁ and A₂ is N and the other is CH;
R₁ is a 5- or 6-membered heteroaryl ring which is optionally substituted on any available carbon atom by one or more R_{1A} substituent groups and on any available nitrogen atom by one or more R_{1B} substituent groups; and wherein:
   each R_{1A} group present is selected from hydroxy, cyano, amino, halo, —C(O)OH, —C(O)NH₂, (1-2C)alkoxy, (1-2C)alkyl, (3-4C)cycloalkyl, (3-4C)cycloalkoxy, —C(O)NH(1-2C)alkyl, —C(O)N[(1-2C)alkyl]₂, —NH(1-2C)alkyl, —N[(1-2C)alkyl]₂, —S(O)_{q}-(1-2C)alkyl (wherein q is 0, 1 or 2), —C(O)(1-2C)alkyl, —C(O)O-(1-2C)alkyl, —N(R_{f})C(O)-(1-2C)alkyl (wherein R_{f} is hydrogen or methyl), —S(O)₂NH(1-2C)alkyl, —S(O)₂N[(1-2C)alkyl]₂, or —NHSO₂-(1-2C)alkyl; and wherein any (1-2C)alkoxy, (1-2C)alkyl, (3-4C)cycloalkyl or (3-4C)cycloalkoxy group is optionally substituted by one or more substituents selected from halo, cyano, hydroxy, (1-2C)alkyl, (1-2C)alkoxy or (1-2C)alkoxy-(1-2C)alkyl];
   and R_{1B} is (1-2C)alkyl or (3-4C)cycloalkyl;
A₃ is selected from CH, CR₃ or N;
A₄ is selected from CH, CR₄ or N;
A₅ and A₆ are both C, or one of A₅ and A₆ is N and the other is C;
A₇ is selected from CH, CR₇ or N;
R₂ is selected from:
   (i) a group R₁ defined above;
   (ii) hydrogen, halo, cyano, (1-4C)alkyl, (1-4C)cyanoalkyl, (1-4C)hydroxyalkyl, (1-4C)haloalkyl, (1-4C)aminoalkyl,
      —[CH₂]₀₋₃-(1-4C)alkoxy,
      —[CH₂]₀₋₃—C(O)NH₂,
      —[CH₂]₀₋₃—C(O)NH(1-4C)alkyl,
      —[CH₂]₀₋₃—C(O)N[(1-4C)alkyl]₂,
      —[CH₂]₀₋₃—NH(1-4C)alkyl,
      —[CH₂]₀₋₃—N[(1-4C)alkyl]₂,
      —[CH₂]₀₋₃—S(O)_{q}-(1-4C)alkyl (wherein q is 0, 1 or 2),
      —[CH₂]₀₋₃—C(O)(1-4C)alkyl,
      —[CH₂]₀₋₃—C(O)OH,
      —[CH₂]₀₋₃—C(O)O-(1-4C)alkyl,
      —[CH₂]₀₋₃—N(R₂ₐ)C(O)-(1-4C)alkyl (wherein R₂ₐ is hydrogen or methyl),
      —[CH₂]₀₋₃—S(O)₂NH(1-4C)alkyl,
      —[CH₂]₀₋₃—S(O)₂N[(1-4C)alkyl]₂,
      —[CH₂]₀₋₃—N(R_{2b})SO₂-(1-4C)alkyl (wherein R_{2b} is hydrogen or methyl),
      a 4- to 7-membered heterocyclyl,
      (3-6C)cycloalkyl,
      (3-6C)cycloalkyl(1-2C)alkyl,
      phenyl, or
      phenyl(1-2C)alkyl;
      and wherein any cycloalkyl, heterocyclyl or phenyl group present is optionally substituted by one or more hydroxy, cyano, amino, halo, —C(O)OH, —C(O)NH₂, (1-2C)alkoxy, (1-2C)alkyl, —C(O)NH(1-2C)alkyl, —C(O)N[(1-2C)alkyl]₂, —NH(1-2C)alkyl, —N[(1-2C)alkyl]₂, —S(O)_{q}-(1-2C)alkyl (wherein q is 0, 1 or 2), —C(O)(1-2C)alkyl, —C(O)O-(1-2C)alkyl, —N(R_{f})C(O)-(1-2C)alkyl (wherein R_{f} is hydrogen or methyl), —S(O)₂NH(1-2C)alkyl, —S(O)₂N[(1-2C)alkyl]₂, or —NHSO₂-(1-2C)alkyl;
R₃ is selected from fluoro, chloro, methyl, methoxy or cyano;
R₄ is selected from fluoro, chloro, methyl, methoxy or cyano;
R₇ is selected from methyl, —NH(1-2C)alkyl or —NH(3-4C)cycloalkyl;
and wherein R₂ is not a group R₁ when A₃, A₄ and A₇ are all CH and A₅ and A₆ are C;
A₈ is selected from CH, CR₈, O, S, NH or N;
A₉ is selected from CH, CR₉, O, S, NH or N;
A₁₀ is selected from CH, CR₁₀, O, S, NH or N;
R₈, R₉ and R₁₀ are selected from methyl, amino, —NH(1-2C)alkyl or —NH(3-4C)cycloalkyl;
and wherein:
   (i) only one of A₈, A₉ and A₁₀ can be NH;
   (ii) one to four of A₅, A₈, A₉ and A₁₀ or A₆, A₈, A₉ and A₁₀ can be N
   (iii) only one of A₈, A₉ or A₁₀ can be 0 or S;
   (iv) when one of A₈, A₉ or A₁₀ are 0 or S, A₅ and/or Aₛ cannot be N.

Particular compounds of the invention include, for example, compounds of the formula I, or pharmaceutically acceptable salts, hydrates and/or solvates thereof, wherein, unless otherwise stated, each of R_{L}, L and R_{R}, and any groups associated therewith, each have any of the meanings defined hereinbefore or are as defined in any one of paragraphs (1) to (163) hereinafter:—
(1) L is linker that separates R_{L} from R_{R} by 8 to 12 bond lengths;
(2) L is linker that separates R_{L} from R_{R} by 9 to 11 bond lengths;
(3) L is linker that separates R_{L} from R_{R} by 10 bond lengths;
(4) L is a linker selected from:
(i) a group of the formula:

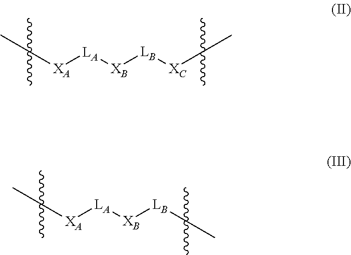

wherein:
   X_{A} is selected from: —O—, —S—, —SO—, —SO₂—, —N(Rₓₐ)—, —C(O)—, —C(O)O—, —OC(O)—, —C(O)N(Rₓₐ)—, —N(Rₓₐ)C(O)—, —N(Rₓₐ₁)C(O)N(Rₓₐ)—, —S(O)₂N(Rₓₐ)— or —N(Rₓₐ)SO₂—, wherein Rₓₐ and Rₓₐ, are selected from hydrogen or methyl;
   L_{A} is a (1-6C)alkylene optionally substituted by one or more R_{LA} substituent groups, or a -[(0-3C)alkylene-(3-6C)cycloalkylene-(0-3C)alkylene]- optionally substituted by one or more R_{LA} substituent groups;
   each R_{LA} group present is selected from hydroxy, halo, (1-2C)alkyl, (1-2C)hydroxyalkyl or (1-2C)haloalkyl;
   X_{B} is selected from: —O—, —S—, —SO—, —SO₂—, —N(R_{xb})—, —C(O)—, —C(O)O—, —OC(O)—, —C(O)N(R_{xb})—, —N(R_{xb})C(O)—, —N(R_{xb})C(O)N(R_{xb})—, —S(O)₂N(R_{xb})— or —N(R_{xb})SO₂—, wherein R_{xb} and R_{xb1} are selected from hydrogen or methyl;
   L_{B} is a (1-6C)alkylene optionally substituted by one or more R_{LB} substituent groups, or a -[(0-3C)alkylene-(3-6C)cycloalkylene-(0-3C)alkylene]-group optionally substituted by one or more R_{LB} substituent groups;
   each R_{LB} group present is selected from hydroxy, halo, (1-2C)alkyl, (1-2C)hydroxyalkyl or (1-2C)haloalkyl;
   X_{c} is selected from: —O—, —S—, —SO—, —SO₂—, —N(R_{xc})—, —C(O)—, —C(O)O—, —OC(O)—, —C(O)N(R_{xc})—, —N(R_{xc1})C(O)—, —N(R_{xc1})C(O)N(R_{xc})—, —S(O)₂N(R_{xc})— or —N(R_{xc})SO₂—, wherein R_{xc} and R_{xc1} are selected from hydrogen or methyl;
(ii) a group of the formula:

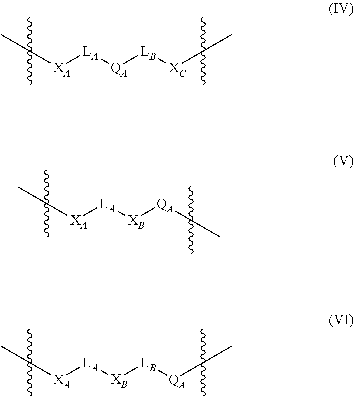

wherein:
X_{A}, L_{A}, X_{B}, L_{B} and X_{C} are as defined above;
Q_{A} is selected from a 5 or 6-membered heteroaryl ring, a 9 or 10-membered bicyclic heteroaryl ring, (3-6C)cycloalkyl, a phenyl, or a 4 to 6 membered heterocyclic ring;
(iii) a group of the formula:

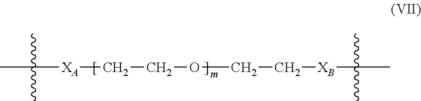

wherein:
X_{A} and X_{B} are each as defined above;
integer m is 1 or 2.
(5) L is a linker selected from:
(i) a group of the formula:

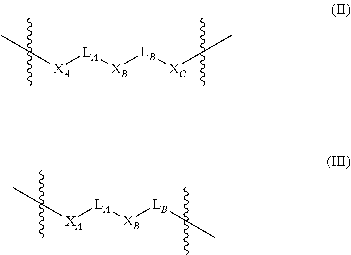

wherein:
   X_{A} is selected from: —O—, —S—, —SO—, —SO₂—, —N(Rₓₐ)—, —C(O)—, —C(O)O—, —OC(O)—, —C(O)N(Rₓₐ)—, —N(Rₓₐ)C(O)—, —N(Rₓₐ₁)C(O)N(Rₓₐ)—, —S(O)₂N(Rₓₐ)— or —N(Rₓₐ)SO₂—, wherein Rₓₐ and Rₓₐ, are selected from hydrogen or methyl;
   L_{A} is a (1-5C)alkylene optionally substituted by one or more R_{LA} substituent groups, or a -[(0-2C)alkylene-(3-6C)cycloalkylene-(0-2C)alkylene]-group optionally substituted by one or more R_{LA} substituent groups;
   each R_{LA} group present is selected from hydroxy, halo, (1-2C)alkyl, (1-2C)hydroxyalkyl or (1-2C)haloalkyl;
   X_{B} is selected from: —O—, —S—, —SO—, —SO₂—, —N(R_{xb})—, —C(O)—, —C(O)O—, —OC(O)—, —C(O)N(R_{xb})—, —N(R_{xb})C(O)—, —N(R_{xb})C(O)N(R_{xb})—, —S(O)₂N(R_{xb})— or —N(R_{xb})SO₂—, wherein R_{xb} and R_{xb1} are selected from hydrogen or methyl;
   L_{B} is a (1-5C)alkylene optionally substituted by one or more R_{LB} substituent groups, or a -[(0-2C)alkylene-(3-6C)cycloalkylene-(0-2C)alkylene]-group optionally substituted by one or more R_{LB} substituent groups;
   each R_{LB} group present is selected from hydroxy, halo, (1-2C)alkyl, (1-2C)hydroxyalkyl or (1-2C)haloalkyl;
   X_{c} is selected from: —O—, —S—, —SO—, —SO₂—, —N(R_{xc})—, —C(O)—, —C(O)O—, —OC(O)—, —C(O)N(R_{xc})—, —N(R_{xc1})C(O)—, —N(R_{xc1})C(O)N(R_{xc})—, —S(O)₂N(R_{xc})— or —N(R_{xc})SO₂—, wherein R_{xc} and R_{xc1} are selected from hydrogen or methyl;
(ii) a group of the formula:

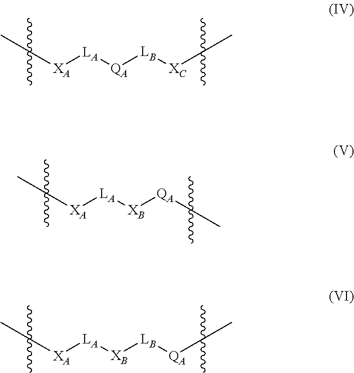

wherein:
X_{A}, L_{A}, X_{B}, L_{B} and X_{C} are as defined above;
Q_{A} is selected from a 5 or 6-membered heteroaryl ring, a 9 or 10-membered bicyclic heteroaryl ring, or a 4 to 6 membered heterocyclic ring;
(iii) a group of the formula:

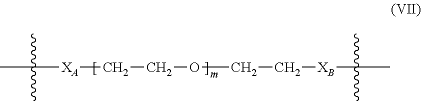

wherein:
X_{A} and X_{B} are each as defined above
integer m is 1.
(6) L is a linker selected from:
(i) a group of the formula:

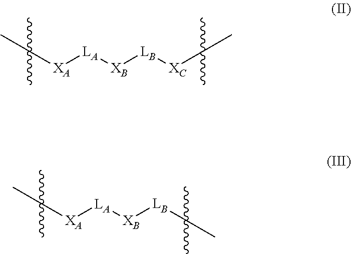

wherein:
   X_{A} is selected from: —O—, —SO₂—, —N(Rₓₐ)—, —C(O)—, —C(O)N(Rₓₐ)—, —N(Rₓₐ)C(O)—, —S(O)₂N(Rₓₐ)— or —N(Rₓₐ)SO₂—, wherein Rₓₐ is selected from hydrogen or methyl;
   L_{A} is a (1-5C)alkylene optionally substituted by one or more R_{LA} substituent groups, or a -[(0-2C)alkylene-(3-6C)cycloalkylene-(0-2C)alkylene]-group optionally substituted by one or more R_{LA} substituent groups;
   each R_{LA} group present is selected from (1-2C)alkyl, (1-2C)hydroxyalkyl or (1-2C)haloalkyl;
   X_{B} is selected from: —O—, —SO₂—, —N(R_{xb})—, —C(O)—, —C(O)N(R_{xb})—, —N(R_{xb1})C(O)—, —S(O)₂N(R_{xb})— or —N(R_{xb})SO₂—, wherein R_{xb} and R_{xb1} are selected from hydrogen or methyl;
   L_{B} is a (1-4C)alkylene optionally substituted by one or more R_{LB} substituent groups, or a -[(0-2C)alkylene-(3-6C)cycloalkylene-(0-2C)alkylene]-group optionally substituted by one or more R_{LB} substituent groups;
   each R_{LB} group present is selected from (1-2C)alkyl, (1-2C)hydroxyalkyl or (1-2C)haloalkyl;
   X_{c} is selected from: —O—, —SO₂—, —N(R_{xc})—, —C(O)—, —C(O)N(R_{xc})—, —N(R_{xc1})C(O)—, —S(O)₂N(R_{xc})— or —N(R_{xc})SO₂—, wherein R_{xc} and R_{xc1} are selected from hydrogen or methyl;
(ii) a group of the formula:

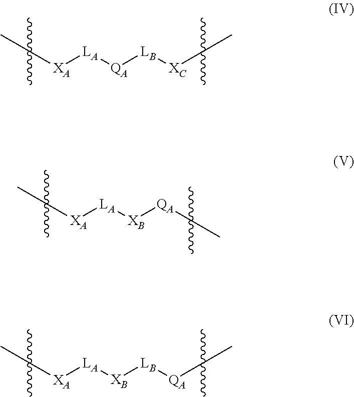

wherein:
X_{A}, L_{A}, X_{B}, L_{B} and X_{C} are as defined above;
Q_{A} is selected from a 5 or 6-membered heteroaryl ring, or a 4 to 6 membered heterocyclic ring;
(iii) a group of the formula:

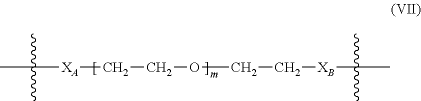

wherein:
X_{A} and X_{B} are each as defined above
integer m is 1.
(7) L is a linker selected from:
(i) a group of the formula:

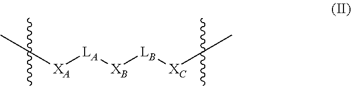

wherein:
   X_{A} is selected from: —O—, —SO₂—, —N(Rₓₐ)—, —C(O)—, —C(O)N(Rₓₐ)—, —N(Rₓₐ)C(O)—, —S(O)₂N(Rₓₐ)— or —N(Rₓₐ)SO₂—, wherein Rₓₐ is selected from hydrogen or methyl;
   L_{A} is a (1-5C)alkylene optionally substituted by one or more R_{LA} substituent groups, or a -[(0-2C)alkylene-(3-6C)cycloalkylene-(0-2C)alkylene]-group optionally substituted by one or more R_{LA} substituent groups;
   each R_{LA} group present is selected from (1-2C)alkyl, (1-2C)hydroxyalkyl or (1-2C)haloalkyl;
   X_{B} is selected from: —O—, —SO₂—, —N(R_{xb})—, —C(O)—, —C(O)N(R_{xb})—, —N(R_{xb1})C(O)—, —S(O)₂N(R_{xb})— or —N(R_{xb})SO₂—, wherein R_{xb} and R_{xb1} are selected from hydrogen or methyl;
   L_{B} is a (1-4C)alkylene optionally substituted by one or more R_{LB} substituent groups, or a -[(0-2C)alkylene-(3-6C)cycloalkylene-(0-2C)alkylene]-group optionally substituted by one or more R_{LB} substituent groups;
   each R_{LB} group present is selected from (1-2C)alkyl, (1-2C)hydroxyalkyl or (1-2C)haloalkyl;
   X_{c} is selected from: —O—, —SO₂—, —N(R_{xc})—, —C(O)—, —C(O)N(R_{xc})—, —N(R_{xc1})C(O)—, —S(O)₂N(R_{xc})— or —N(R_{xc})SO₂—, wherein R_{xc} and R_{xc1} are selected from hydrogen or methyl;
(ii) a group of the formula:

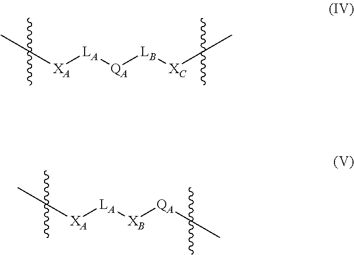

wherein:
X_{A}, L_{A}, X_{B}, L_{B} and X_{C} are as defined above;
Q_{A} is selected from a 5 or 6-membered heteroaryl ring, or a 4 to 6 membered heterocyclic ring;
(8) L is a linker selected from:
(i) a group of the formula:

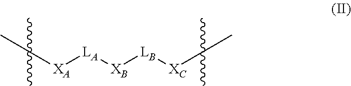

wherein:
   X_{A} is selected from: —O—, —SO₂—, —N(Rₓₐ)—, —C(O)N(Rₓₐ)—, —N(Rₓₐ)C(O)—, —S(O)₂N(Rₓₐ)— or —N(Rₓₐ)SO₂—, wherein Rₓₐ is selected from hydrogen or methyl;
   L_{A} is a (1-5C)alkylene optionally substituted by one or more R_{LA} substituent groups, or a -[(0-1C)alkylene-(4-6C)cycloalkylene-(0-1C)alkylene]-group,
   each R_{LA} group present is selected from (1-2C)alkyl, (1-2C)hydroxyalkyl or (1-2C)haloalkyl;
   X_{B} is selected from: —O—, —SO₂—, —N(R_{xb})—, —C(O)N(R_{xb})—, —N(R_{xb1})C(O)—, —S(O)₂N(R_{xb})— or —N(R_{xb})SO₂—, wherein R_{xb} and R_{xb1} are selected from hydrogen or methyl;
   L_{B} is a (1-4C)alkylene optionally substituted by one or more R_{LB} substituent groups,
   each R_{LB} group present is selected from (1-2C)alkyl, (1-2C)hydroxyalkyl or (1-2C)haloalkyl;
   X_{c} is selected from: —O—, —SO₂—, —N(R_{xc})—, —C(O)N(R_{xc})—, —N(R_{xc1})C(O)—, —S(O)₂N(R_{xc})— or —N(R_{xc})SO₂—, wherein R_{xc} and R_{xc1} are selected from hydrogen or methyl;
(ii) a group of the formula:

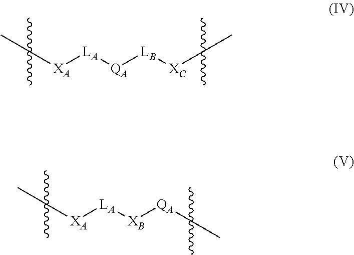

wherein:
X_{A}, L_{A}, X_{B}, L_{B} and X_{C} are as defined above;
Q_{A} is selected from a 5 or 6-membered heteroaryl ring, or a 4 to 6 membered heterocyclic ring;
(9) L is a linker selected from:
(i) a group of the formula:

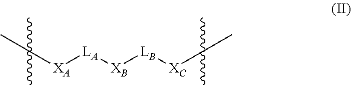

wherein:
   X_{A} is selected from: —O—, —N(Rₓₐ)—, —C(O)N(Rₓₐ)—, or —N(Rₓₐ)C(O)—, wherein Rₓₐ is selected from hydrogen or methyl;
   L_{A} is a (2-5C)alkylene optionally substituted by one or more R_{LA} substituent groups, or a -[(4-6C)cycloalkylene-(0-1C)alkylene]-group, each RLA group present is (1-2C)alkyl;
   X_{B} is selected from: —O—, —N(R_{xb})—, —C(O)N(R_{xb})—, or —N(R_{xb1})C(O)—, wherein R_{xb} and R_{xb1} are selected from hydrogen or methyl;
   L_{B} is a (1-4C)alkylene optionally substituted by one or more R_{LB} substituent groups,
   each R_{LB} group present is (1-2C)alkyl;
   X_{c} is selected from: —O—, —N(R_{xc})—, —C(O)N(R_{xc})— or —N(R_{xc1})C(O)—, wherein R_{xc} and R_{xc1} are selected from hydrogen or methyl;
(ii) a group of the formula:

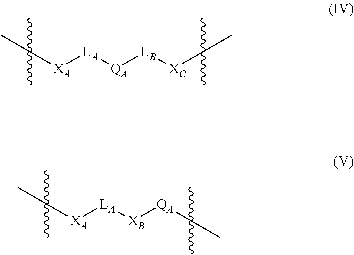

wherein:
X_{A}, L_{A}, X_{B}, L_{B} and X_{C} are as defined above;
Q_{A} is selected from a 5 or 6-membered heteroaryl ring, or a 4 to 6 membered heterocyclic ring;
(10) L is a linker selected from:
(i) a group of the formula:

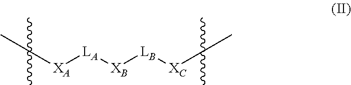

wherein:
   X_{A} is selected from: —O—, —N(H)—, or —N(Me)-;
   L_{A} is a (2-4C)alkylene optionally substituted by one or more R_{LA} substituent groups, or a -[(4-5C)cycloalkylene-(0-1C)alkylene]-group,
   each RLA group present is (1-2C)alkyl;
   X_{B} is selected from: —O—, —N(H)—, —N(Me)-, —C(O)N(H)-, —C(O)N(Me)-, —N(H)C(O)— or —N(Me)C(O)—;
   L_{B} is a (1-4C)alkylene optionally substituted by one or more R_{LB} substituent groups,
   each R_{LB} group present is (1-2C)alkyl;
   X_{c} is selected from: —O—, —N(H)—, —N(Me)-, —C(O)N(H)—, —C(O)N(Me)-, —N(H)C(O)— or —N(Me)C(O)—;
(ii) a group of the formula:

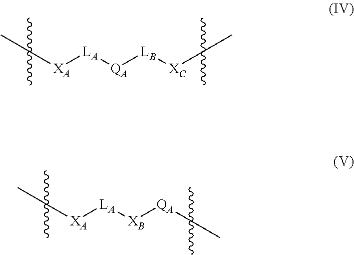

wherein:
X_{A}, L_{A}, X_{B}, L_{B} and X_{C} are as defined above;
Q_{A} is selected from a 5-membered heteroaryl ring, or a 4-membered heterocyclic ring;
(11) L is a linker selected from:
(i) a group of the formula:

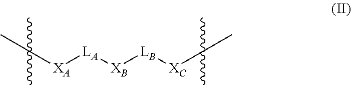

wherein:
   X_{A} is selected from: —N(H)—, or —N(Me)-;
   L_{A} is a (2-4C)alkylene optionally substituted by a methyl group, or L_{A} is a -[cyclobutylene-(0-1C)alkylene]-group;
   X_{B} is selected from: —O—, —N(H)—, —N(Me)-, —C(O)N(H)—, or —C(O)N(Me)-;
   L_{B} is a (1-4C)alkylene optionally substituted by a methyl group;
   X_{c} is selected from: —O—, —N(H)—, —N(Me)-, —C(O)N(H)— or —C(O)N(Me)-;
(ii) a group of the formula:

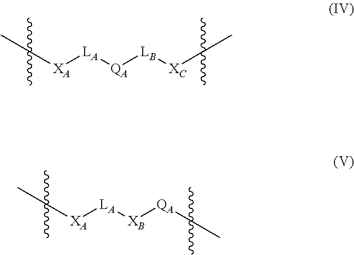

wherein:
X_{A}, L_{A}, X_{B}, L_{B} and X_{C} are as defined above;
Q_{A} is selected from an oxazole group, triazole group or a azetidine group;
(12) L is a linker selected from:

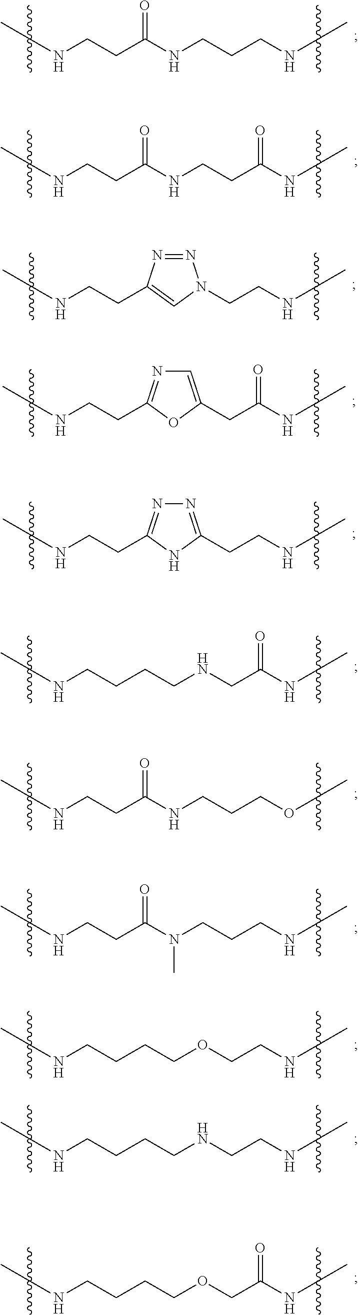

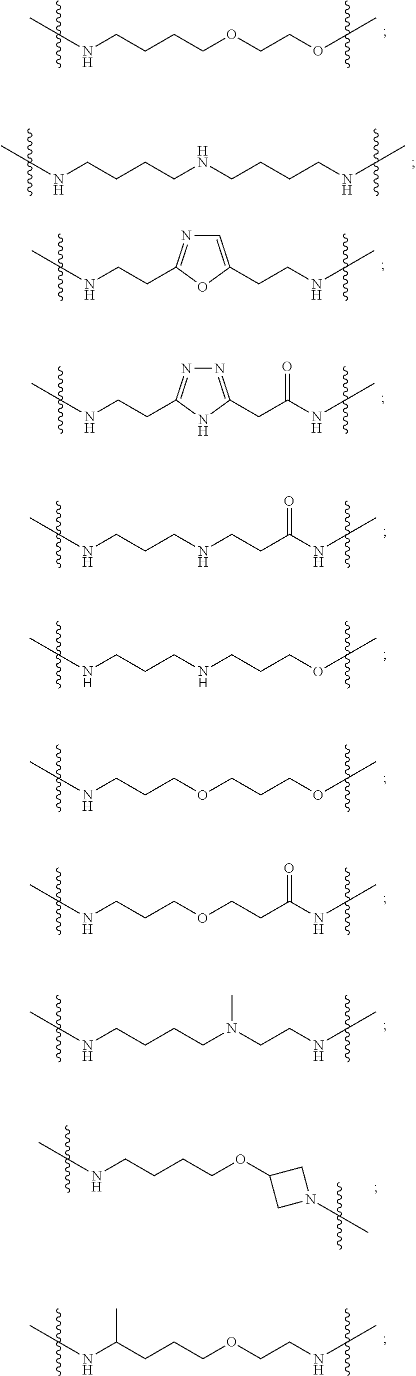

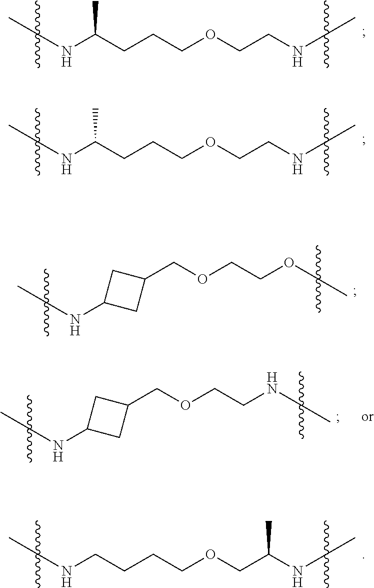 [SMILES: *NCCOCCC[C@H](C)N* ; *NCCOCCC[C@@H](C)N* ; *NC1CC(COCCO*)C1 ; *NCCOCC1CC(N*)C1 ; *NCCCCOC[C@@H](C)N*]

(13) L is a linker selected from:

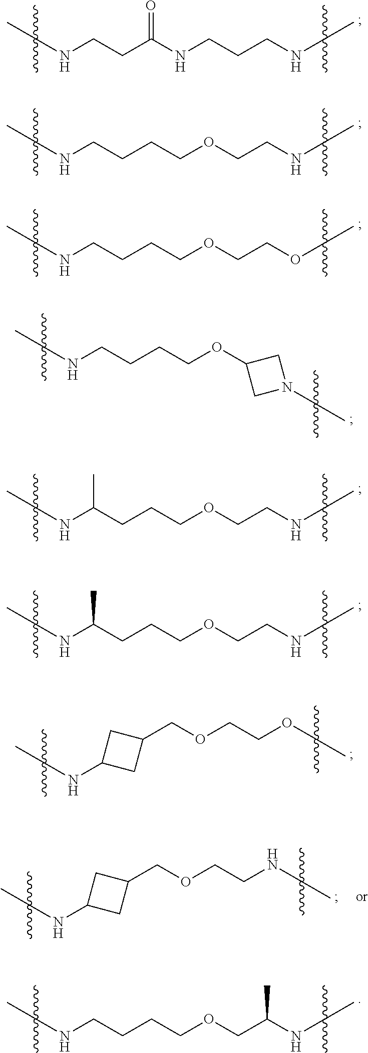

(14) R_{L} is selected from one of formulae Ic, Id, Ie, If or Ii shown below:

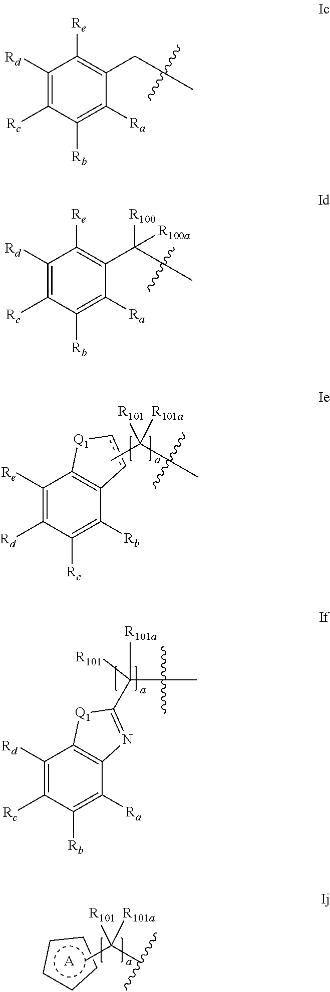

(15) R_{L} is selected from one of formulae Ic, Ie or If shown below:

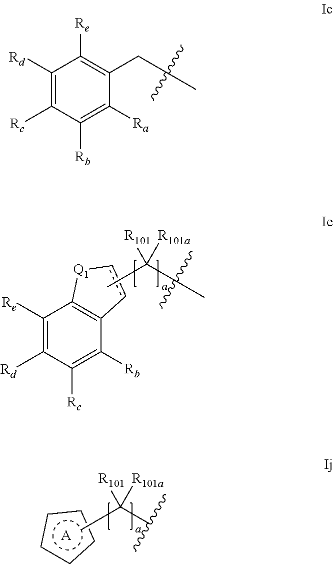

(16) R_{L} is selected from one of formulae Ic or Ie shown below:

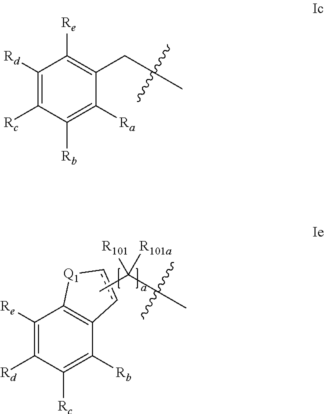

(17) R_{L} is selected from one of formulae shown below:

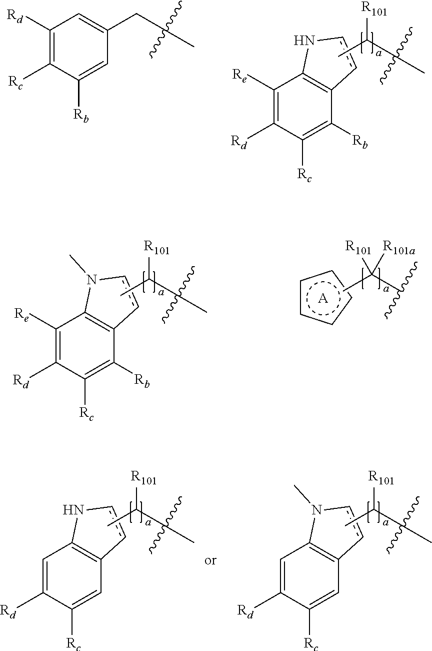

wherein:
[symbol] denotes the point of attachment; and
[symbol] denotes an optional double bond;
(18) R_{L} is selected from one of formulae shown below:

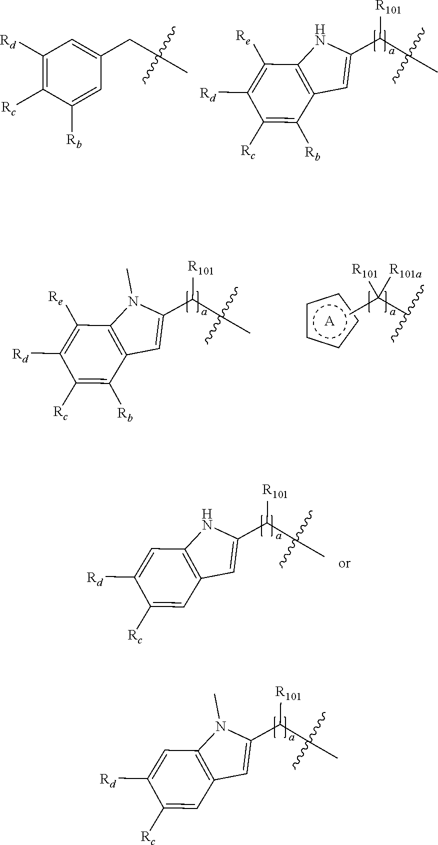

wherein:
[symbol] denotes the point of attachment;
(19) R_{L} is selected from one of formulae shown below:

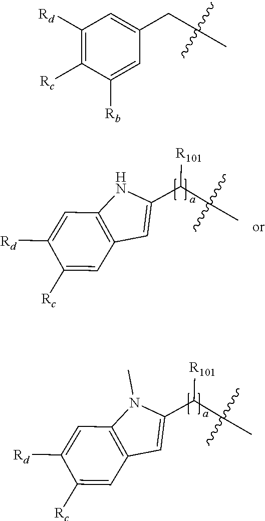

wherein:
[symbol] denotes the point of attachment;
(20) Rₐ and Rₑ are each independently selected from hydrogen, methyl, fluoro, chloro or bromo;
(21) Rₐ and Rₑ are each independently selected from hydrogen, fluoro, chloro or bromo;
(22) Rₐ and Rₑ are each independently selected from hydrogen or chloro;
(23) Rₐ and Rₑ are both hydrogen;
(24) R_{b} and R_{d} are each independently selected from hydrogen, halo, cyano, (1-4C)alkyl, (3-6C)cycloalkyl,
   —[CH₂]₀₋₂-(1-4C)alkoxy,
   —[CH₂]₀₋₂—C(O)NH₂,
   —[CH₂]₀₋₂—C(O)NH(1-4C)alkyl,
   —[CH₂]₀₋₂—C(O)N[(1-4C)alkyl]₂,
   —[CH₂]₀₋₂—NH₂,
   —[CH₂]₀₋₂—NH(1-4C)alkyl,
   —[CH₂]₀₋₂—N[(1-4C)alkyl]₂,
   —[CH₂]₀₋₂—S(O)_{q}-(1-4C)alkyl (wherein q is 0, 1 or 2),
   —[CH₂]₀₋₂—C(O)(1-4C)alkyl,
   —[CH₂]₀₋₂—C(O)OH,
   —[CH₂]₀₋₂—C(O)O-(1-4C)alkyl,
   —[CH₂]₀₋₂—N(R_{f})C(O)-(1-4C)alkyl (wherein R_{f} is hydrogen or methyl),
   —[CH₂]₀₋₂—S(O)₂NH(1-4C)alkyl,
   —[CH₂]₀₋₂—S(O)₂N[(1-4C)alkyl]₂,
   —[CH₂]₀₋₂—N(R₉)SO₂-(1-4C)alkyl (wherein R₉ is hydrogen or methyl),
   a group of the formula:

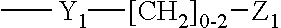

wherein Y₁ is absent, —O—, —NH—, —NMe-, —S—, —S(O)— or —S(O)₂—; and
Z₁ is (3-6C)cycloalkyl, phenyl, a 4- to 6-membered heterocyclyl or 5 or 6-membered heteroaryl;
and wherein:
any alkyl, alkoxy, cycloalkyl or —[CH₂]— moiety within a R_{b} and R_{d} substituent group is optionally substituted by one or more substituents selected from halo, hydroxy, cyano, amino, —C(O)OH, —C(O)NH₂, (1-2C)alkoxy, or (3-4C)cycloalkoxy; and
Z₁ is optionally substituted by one or more substituents selected from: halo, hydroxy, cyano, amino, —C(O)OH, —C(O)NH₂, (1-2C)alkoxy, (1-2C)alkyl, (3-4C)cycloalkyl, (3-4C)cycloalkoxy, —C(O)NH(1-2C)alkyl, —C(O)N[(1-2C)alkyl]₂, —NH(1-2C)alkyl, —N[(1-2C)alkyl]₂, —S(O)_{q}-(1-2C)alkyl (wherein q is 0, 1 or 2), —C(O)(1-2C)alkyl, —C(O)O-(1-2C)alkyl, —N(R_{f})C(O)-(1-2C)alkyl, —S(O)₂NH(1-2C)alkyl, —S(O)₂N[(1-2C)alkyl]₂, or —NHSO₂-(1-2C)alkyl, and wherein any (1-2C)alkoxy, (1-2C)alkyl, (3-4C)cycloalkyl or (3-4C)cycloalkoxy group is optionally substituted by one or more substituents selected from halo, cyano, hydroxy, (1-2C)alkyl, (1-2C)alkoxy or (1-2C)alkoxy-(1-2C)alkyl;
(25) R_{b} and R_{d} are each independently selected from hydrogen, halo, cyano, (1-4C)alkyl, (3-6C)cycloalkyl,
   —[CH₂]₀₋₂-(1-4C)alkoxy,
   —[CH₂]₀₋₂—C(O)NH₂,
   —[CH₂]₀₋₂—C(O)NH(1-2C)alkyl,
   —[CH₂]₀₋₂—C(O)N[(1-2C)alkyl]₂,
   —[CH₂]₀₋₂—NH₂,
   —[CH₂]₀₋₂—NH(1-2C)alkyl,
   —[CH₂]₀₋₂—N[(1-2C)alkyl]₂,
   —[CH₂]₀₋₂—S(O)_{q}-(1-2C)alkyl (wherein q is 0, 1 or 2),
   —[CH₂]₀₋₂—C(O)(1-2C)alkyl,
   —[CH₂]₀₋₂—C(O)OH,
   —[CH₂]₀₋₂—C(O)O-(1-2C)alkyl,
   —[CH₂]₀₋₂—N(R_{f})C(O)-(1-2C)alkyl (wherein R_{f} is hydrogen or methyl),
   —[CH₂]₀₋₂—S(O)₂NH(1-2C)alkyl,
—[CH₂]₀₋₂—S(O)₂N[(1-2C)alkyl]₂,
   —[CH₂]₀₋₂—N(R₉)SO₂-(1-2C)alkyl (wherein R₉ is hydrogen or methyl),
   a group of the formula:

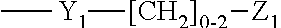

wherein Y₁ is absent, —O—, —NH—, —NMe-, —S—, —S(O)— or —S(O)₂—; and
Z₁ is (3-6C)cycloalkyl, phenyl, a 4- to 6-membered heterocyclyl or 5 or 6-membered heteroaryl;
and wherein:
any alkyl, alkoxy, cycloalkyl or —[CH₂]— moiety within a R_{b} and R_{d} substituent group is optionally substituted by one or more substituents selected from halo, hydroxy, cyano, amino, —C(O)OH, —C(O)NH₂, (1-2C)alkoxy, or (3-4C)cycloalkoxy; and
Z₁ is optionally substituted by one or more substituents selected from: halo, hydroxy, cyano, amino, —C(O)OH, —C(O)NH₂, (1-2C)alkoxy, (1-2C)alkyl, (3-4C)cycloalkyl, (3-4C)cycloalkoxy, —C(O)NH(1-2C)alkyl, —C(O)N[(1-2C)alkyl]₂, —NH(1-2C)alkyl, —N[(1-2C)alkyl]₂, —S(O)_{q}-(1-2C)alkyl (wherein q is 0, 1 or 2), —C(O)(1-2C)alkyl, —C(O)O-(1-2C)alkyl, —N(R_{f})C(O)-(1-2C)alkyl, —S(O)₂NH(1-2C)alkyl, —S(O)₂N[(1-2C)alkyl]₂, or —NHSO₂-(1-2C)alkyl, and wherein any (1-2C)alkoxy, (1-2C)alkyl, (3-4C)cycloalkyl or (3-4C)cycloalkoxy group is optionally substituted by one or more substituents selected from halo, cyano, hydroxy, (1-2C)alkyl, (1-2C)alkoxy or (1-2C)alkoxy-(1-2C)alkyl;
(26) R_{b} and R_{d} are each independently selected from hydrogen, halo, cyano, (1-4C)alkyl, (3-6C)cycloalkyl,
   —[CH₂]₀₋₁-(1-4C)alkoxy,
   —[CH₂]₀₋₁—C(O)NH₂,
   —[CH₂]₀₋₁—C(O)NH(1-2C)alkyl,
   —[CH₂]₀₋₁—C(O)N[(1-2C)alkyl]₂,
   —[CH₂]₀₋₁—NH₂,
   —[CH₂]₀₋₁—NH(1-2C)alkyl,
   —[CH₂]₀₋₁—N[(1-2C)alkyl]₂,
   —[CH₂]₀₋₁—S(O)_{q}-(1-2C)alkyl (wherein q is 0, 1 or 2),
   —[CH₂]₀₋₁—C(O)(1-2C)alkyl,
   —[CH₂]₀₋₁—C(O)OH,
   —[CH₂]₀₋₁—C(O)O-(1-2C)alkyl,
   —[CH₂]₀₋₁—N(H)C(O)-(1-2C)alkyl,
   —[CH₂]₀₋₁—S(O)₂NH(1-2C)alkyl,
   —[CH₂]₀₋₁—S(O)₂N[(1-2C)alkyl]₂,
   —[CH₂]₀₋₁—N(H)SO₂-(1-2C)alkyl,
   a group of the formula:

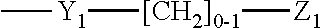

wherein Y₁ is absent, —O—, —NH—, —NMe-, —S—, —S(O)— or —S(O)₂—; and
Z₁ is (3-6C)cycloalkyl, phenyl, a 4- to 6-membered heterocyclyl or 5 or 6-membered heteroaryl;
and wherein:
any alkyl, alkoxy, cycloalkyl or —[CH₂]— moiety within a R_{b} and R_{d} substituent group is optionally substituted by one or more substituents selected from halo, hydroxy, cyano, amino, —C(O)OH, —C(O)NH₂, (1-2C)alkoxy, or (3-4C)cycloalkoxy; and
Z₁ is optionally substituted by one or more substituents selected from: halo, hydroxy, cyano, amino, —C(O)OH, —C(O)NH₂, (1-2C)alkoxy, (1-2C)alkyl, (3-4C)cycloalkyl, (3-4C)cycloalkoxy, —C(O)NH(1-2C)alkyl, —C(O)N[(1-2C)alkyl]₂, —NH(1-2C)alkyl, —N[(1-2C)alkyl]₂, —S(O)_{q}-(1-2C)alkyl (wherein q is 0, 1 or 2), —C(O)(1-2C)alkyl, —C(O)O-(1-2C)alkyl, —N(R_{f})C(O)-(1-2C)alkyl, —S(O)₂NH(1-2C)alkyl, —S(O)₂N[(1-2C)alkyl]₂, or —NHSO₂-(1-2C)alkyl, and wherein any (1-2C)alkoxy, (1-2C)alkyl, (3-4C)cycloalkyl or (3-4C)cycloalkoxy group is optionally substituted by one or more substituents selected from halo, cyano, hydroxy, (1-2C)alkyl, (1-2C)alkoxy or (1-2C)alkoxy-(1-2C)alkyl;
(27) R_{b} and R_{d} are each independently selected from hydrogen, halo, cyano, (1-4C)alkyl, (3-6C)cycloalkyl,
   —[CH₂]₀₋₁-(1-4C)alkoxy,
   —[CH₂]₀₋₁—C(O)NH₂,
   —[CH₂]₀₋₁—C(O)NH(1-2C)alkyl,
   —[CH₂]₀₋₁—NH₂,
   —[CH₂]₀₋₁—NH(1-2C)alkyl,
   —[CH₂]₀₋₁—C(O)(1-2C)alkyl,
   —[CH₂]₀₋₁—C(O)OH,
   —[CH₂]₀₋₁—C(O)O-(1-2C)alkyl,
   —[CH₂]₀₋₁—N(H)C(O)-(1-2C)alkyl,
   a group of the formula:

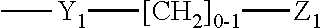

wherein Y₁ is absent, —O—, —NH—, —NMe-, —S—, —S(O)— or —S(O)₂—; and
Z₁ is (3-6C)cycloalkyl, phenyl, a 4- to 6-membered heterocyclyl or 5 or 6-membered heteroaryl;
and wherein:
any alkyl, alkoxy, cycloalkyl or —[CH₂]— moiety within a R_{b} and R_{d} substituent group is optionally substituted by one or more substituents selected from halo, hydroxy, cyano, amino, —C(O)OH, —C(O)NH₂, (1-2C)alkoxy, or (3-4C)cycloalkoxy; and
Z₁ is optionally substituted by one or more substituents selected from: halo, hydroxy, cyano, amino, —C(O)OH, —C(O)NH₂, (1-2C)alkoxy, (1-2C)alkyl, (3-4C)cycloalkyl, (3-4C)cycloalkoxy, —C(O)NH(1-2C)alkyl, —C(O)N[(1-2C)alkyl]₂, —NH(1-2C)alkyl, —N[(1-2C)alkyl]₂, —S(O)_{q}-(1-2C)alkyl (wherein q is 0, 1 or 2), —C(O)(1-2C)alkyl, —C(O)O-(1-2C)alkyl, —N(R_{f})C(O)-(1-2C)alkyl, —S(O)₂NH(1-2C)alkyl, —S(O)₂N[(1-2C)alkyl]₂, or —NHSO₂-(1-2C)alkyl, and wherein any (1-2C)alkoxy, (1-2C)alkyl, (3-4C)cycloalkyl or (3-4C)cycloalkoxy group is optionally substituted by one or more substituents selected from halo, cyano, hydroxy, (1-2C)alkyl, (1-2C)alkoxy or (1-2C)alkoxy-(1-2C)alkyl;
(28) R_{b} and R_{d} are each independently selected from hydrogen, halo, cyano, (1-4C)alkyl, (3-6C)cycloalkyl,
   —[CH₂]₀₋₁-(1-4C)alkoxy,
   —[CH₂]₀₋₁—C(O)NH₂,
   —[CH₂]₀₋₁—C(O)NH(1-2C)alkyl,
   —[CH₂]₀₋₁—C(O)(1-2C)alkyl,
   —[CH₂]₀₋₁—C(O)OH,
   a group of the formula:

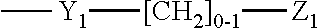

wherein Y₁ is absent, —O—, —NH—, —NMe-, —S—, —S(O)— or —S(O)₂—; and
Z₁ is (3-6C)cycloalkyl, phenyl, a 4- to 6-membered heterocyclyl or 5 or 6-membered heteroaryl;
and wherein:
any alkyl, alkoxy, cycloalkyl or —[CH₂]— moiety within a R_{b} and R_{d} substituent group is optionally substituted by one or more substituents selected from halo, hydroxy, cyano, amino, —C(O)OH, —C(O)NH₂, (1-2C)alkoxy, or (3-4C)cycloalkoxy; and
Z₁ is optionally substituted by one or more substituents selected from: halo, hydroxy, cyano, amino, —C(O)OH, —C(O)NH₂, (1-2C)alkoxy, (1-2C)alkyl, (3-4C)cycloalkyl, (3-4C)cycloalkoxy, —C(O)NH(1-2C)alkyl, —C(O)N[(1-2C)alkyl]₂, —NH(1-2C)alkyl, —N[(1-2C)alkyl]₂, —C(O)(1-2C)alkyl, —C(O)O-(1-2C)alkyl or —N(R_{f})C(O)-(1-2C)alkyl, and wherein any (1-2C)alkoxy, (1-2C)alkyl, (3-4C)cycloalkyl or (3-4C)cycloalkoxy group is optionally substituted by one or more substituents selected from halo, cyano, hydroxy, (1-2C)alkyl, (1-2C)alkoxy or (1-2C)alkoxy-(1-2C)alkyl;
(29) R_{b} and R_{d} are each independently selected from hydrogen, halo, cyano, (1-4C)alkyl,
   —[CH₂]₀₋₁-(1-4C)alkoxy,
   —[CH₂]₀₋₁—C(O)NH₂,
   a group of the formula:

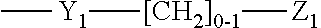

wherein Y₁ is absent, —O—, —NH— or —NMe-; and
Z₁ is (4-6C)cycloalkyl, phenyl, a 4- to 6-membered heterocyclyl or 5 or 6-membered heteroaryl;
and wherein:
any alkyl, alkoxy, cycloalkyl or —[CH₂]— moiety within a R_{b} and R_{d} substituent group is optionally substituted by one or more substituents selected from halo, hydroxy, cyano, amino, —C(O)OH, —C(O)NH₂, (1-2C)alkoxy, or (3-4C)cycloalkoxy; and
Z₁ is optionally substituted by one or more substituents selected from: halo, hydroxy, cyano, amino, —C(O)OH, —C(O)NH₂, (1-2C)alkoxy, (1-2C)alkyl, (3-4C)cycloalkyl, (3-4C)cycloalkoxy, —C(O)NH(1-2C)alkyl, —C(O)N[(1-2C)alkyl]₂, —NH(1-2C)alkyl, —N[(1-2C)alkyl]₂, —C(O)(1-2C)alkyl, —C(O)O-(1-2C)alkyl or —N(R_{f})C(O)-(1-2C)alkyl, and wherein any (1-2C)alkoxy, (1-2C)alkyl, (3-4C)cycloalkyl or (3-4C)cycloalkoxy group is optionally substituted by one or more substituents selected from halo, cyano, hydroxy, (1-2C)alkyl, (1-2C)alkoxy or (1-2C)alkoxy-(1-2C)alkyl;
(30) R_{b} and R_{d} are each independently selected from hydrogen, halo, cyano, (1-3C)alkyl, (1-3C)alkoxy, —C(O)NH₂,
   a group of the formula:

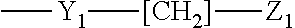

wherein Y₁ is absent; and
Z₁ is 5 or 6-membered heteroaryl;
and wherein:
any alkyl, alkoxy, cycloalkyl or —[CH₂]— moiety within a R_{b} and R_{d} substituent group is optionally substituted by one or more substituents selected from halo, hydroxy, cyano, amino, —C(O)OH, —C(O)NH₂, (1-2C)alkoxy, or (3-4C)cycloalkoxy; and
Z₁ is optionally substituted by one or more substituents selected from: halo, hydroxy, cyano, amino, —C(O)OH, —C(O)NH₂, (1-2C)alkoxy, (1-2C)alkyl, (3-4C)cycloalkyl, (3-4C)cycloalkoxy, —C(O)NH(1-2C)alkyl, —C(O)N[(1-2C)alkyl]₂, —NH(1-2C)alkyl, —N[(1-2C)alkyl]₂, —C(O)(1-2C)alkyl, —C(O)O-(1-2C)alkyl or —N(R_{f})C(O)-(1-2C)alkyl, and wherein any (1-2C)alkoxy, (1-2C)alkyl, (3-4C)cycloalkyl or (3-4C)cycloalkoxy group is optionally substituted by one or more substituents selected from halo, cyano, hydroxy, (1-2C)alkyl, (1-2C)alkoxy or (1-2C)alkoxy-(1-2C)alkyl;
(31) R_{b} and R_{d} are each independently selected from hydrogen, fluoro, chloro, bromo, cyano, methyl, ethyl, methoxy, ethoxy, —C(O)NH₂, —CH₂-oxazole, —CH₂OH, —CH₂OCH₃, —CH₂CN, —CH₂CH₂OH, —CF3, —OCF₃, —O—CH₂CH₂OH, —O—CH₂CF₃, —CH₂—C(O)NH₂, —CH(CH₃)CN or —C(CH₃)₂CN;
(32) R_{b} and R_{d} are each independently selected from hydrogen, fluoro, chloro cyano, methyl, ethyl, methoxy, ethoxy, —C(O)NH₂, —CH₂-oxazole, —CH₂OH, —CH₂CN, —CH₂CH₂OH, —CF3, —OCF₃, —O—CH₂CH₂OH or —CH₂—C(O)NH₂;
(33) One of R_{b} and R_{d} is hydrogen or halogen and the other is selected from any one of the options defined in paragraphs (24) to (32);
(34) One of R_{b} and R_{d} is hydrogen or halogen and the other is selected from hydrogen, fluoro, chloro cyano, methyl, ethyl, methoxy, ethoxy, —C(O)NH₂, —CH₂-oxazole, —CH₂OH, —CH₂CN, —CH₂CH₂OH, —CF3, —OCF₃, —O—CH₂CH₂OH or —CH₂—C(O)NH₂;
(35) R_{c} is selected from hydrogen, halo, cyano, —C(O)NH₂, (1-4C)alkyl, (3-6C)cycloalkyl
   —[CH₂]₀₋₂-(1-4C)alkoxy,
   —[CH₂]₀₋₂-(3-6C)cycloalkoxy,
   —[CH₂]₀₋₂—C(O)NH₂,
   —[CH₂]₀₋₂—C(O)NH(1-2C)alkyl,
   —[CH₂]₀₋₂—C(O)N[(1-2C)alkyl]₂,
—[CH₂]₀₋₂—NH₂,
   —[CH₂]₀₋₂—NH(1-2C)alkyl,
   —[CH₂]₀₋₂—N[(1-2C)alkyl]₂,
   —[CH₂]₀₋₂—S(O)_{q}-(1-2C)alkyl (wherein q is 0, 1 or 2),
   —[CH₂]₀₋₂—C(O)(1-2C)alkyl,
   —[CH₂]₀₋₂—C(O)OH,
   —[CH₂]₀₋₂—C(O)O-(1-2C)alkyl,
   —[CH₂]₀₋₂—N(Rn)C(O)-(1-2C)alkyl (wherein Rₕ is hydrogen or methyl),
   —[CH₂]₀₋₂—S(O)₂NH(1-2C)alkyl,
   —[CH₂]₀₋₂—S(O)₂N[(1-2C)alkyl]₂,
   —[CH₂]₀₋₂—N(Rᵢ)SO₂-(1-2C)alkyl (wherein Rᵢ is hydrogen or methyl),
   a group of the formula:

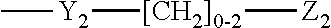

wherein Y₂ is absent, —O—, —NH—, —NMe-, —S—, —S(O)— or —S(O)₂—; and
Z₂ is (3-6C)cycloalkyl, phenyl, a 4- to 6-membered heterocyclyl or 5 or 6-membered heteroaryl;
and wherein:
any alkyl, alkoxy, cycloalkyl or —[CH₂]— moiety within a R_{c} substituent group is optionally substituted by one or more substituents selected from halo, hydroxy, cyano, amino, —C(O)OH, —C(O)NH₂, (1-2C)alkoxy, or (3-4C)cycloalkoxy; and
Z₂ is optionally substituted by one or more substituents selected from: halo, hydroxy, cyano, amino, —C(O)OH, —C(O)NH₂, (1-2C)alkoxy, (1-2C)alkyl, (3-4C)cycloalkyl, (3-4C)cycloalkoxy, —C(O)NH(1-2C)alkyl, —C(O)N[(1-2C)alkyl]₂, —NH(1-2C)alkyl, —N[(1-2C)alkyl]₂, —S(O)_{q}-(1-2C)alkyl (wherein q is 0, 1 or 2), —C(O)(1-2C)alkyl, —C(O)O-(1-2C)alkyl, —N(R_{f})C(O)-(1-2C)alkyl, —S(O)₂NH(1-2C)alkyl, —S(O)₂N[(1-2C)alkyl]₂, or —NHSO₂-(1-2C)alkyl, and wherein any (1-2C)alkoxy, (1-2C)alkyl, (3-4C)cycloalkyl or (3-4C)cycloalkoxy group is optionally substituted by one or more substituents selected from halo, cyano, hydroxy, (1-2C)alkyl, (1-2C)alkoxy or (1-2C)alkoxy-(1-2C)alkyl;
(36) R_{c} is selected from hydrogen, halo, cyano, —C(O)NH₂, (1-4C)alkyl, (3-6C)cycloalkyl
   —[CH₂]₀₋₁-(1-4C)alkoxy,
   —[CH₂]₀₋₁-(3-6C)cycloalkoxy,
   —[CH₂]₀₋₁—C(O)NH₂,
   —[CH₂]₀₋₁—C(O)NH(1-2C)alkyl,
   —[CH₂]₀₋₁—C(O)N[(1-2C)alkyl]₂,
   —[CH₂]₀₋₁—NH₂,
   —[CH₂]₀₋₁—NH(1-2C)alkyl,
   —[CH₂]₀₋₁—N[(1-2C)alkyl]₂,
   —[CH₂]₀₋₁—S(O)_{q}-(1-2C)alkyl (wherein q is 0, 1 or 2),
   —[CH₂]₀₋₁—C(O)(1-2C)alkyl,
   —[CH₂]₀₋₁—C(O)OH,
   —[CH₂]₀₋₁—C(O)O-(1-2C)alkyl,
   —[CH₂]₀₋₁—N(Rₕ)C(O)-(1-2C)alkyl (wherein Rₕ is hydrogen or methyl),
   —[CH₂]₀₋₁—S(O)₂NH(1-2C)alkyl,
   —[CH₂]₀₋₁—S(O)₂N[(1-2C)alkyl]₂,
   —[CH₂]₀₋₁—N(Rᵢ)SO₂-(1-2C)alkyl (wherein Rᵢ is hydrogen or methyl),
   a group of the formula:

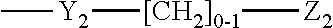

wherein Y₂ is absent, —O—, —NH—, —NMe-, —S—, —S(O)— or —S(O)₂—; and
Z₂ is (3-6C)cycloalkyl, phenyl, a 4- to 6-membered heterocyclyl or 5 or 6-membered heteroaryl;
and wherein:
any alkyl, alkoxy, cycloalkyl or —[CH₂]— moiety within a R_{c} substituent group is optionally substituted by one or more substituents selected from halo, hydroxy, cyano, amino, —C(O)OH, —C(O)NH₂, (1-2C)alkoxy, or (3-4C)cycloalkoxy; and
Z₂ is optionally substituted by one or more substituents selected from: halo, hydroxy, cyano, amino, —C(O)OH, —C(O)NH₂, (1-2C)alkoxy, (1-2C)alkyl, (3-4C)cycloalkyl, (3-4C)cycloalkoxy, —C(O)NH(1-2C)alkyl, —C(O)N[(1-2C)alkyl]₂, —NH(1-2C)alkyl, —N[(1-2C)alkyl]₂, —S(O)_{q}-(1-2C)alkyl (wherein q is 0, 1 or 2), —C(O)(1-2C)alkyl, —C(O)O-(1-2C)alkyl, —N(R_{f})C(O)-(1-2C)alkyl, —S(O)₂NH(1-2C)alkyl, —S(O)₂N[(1-2C)alkyl]₂, or —NHSO₂-(1-2C)alkyl, and wherein any (1-2C)alkoxy, (1-2C)alkyl, (3-4C)cycloalkyl or (3-4C)cycloalkoxy group is optionally substituted by one or more substituents selected from halo, cyano, hydroxy, (1-2C)alkyl, (1-2C)alkoxy or (1-2C)alkoxy-(1-2C)alkyl;
(37) R_{c} is selected from hydrogen, halo, cyano, —C(O)NH₂, (1-4C)alkyl, (3-6C)cycloalkyl
   —[CH₂]₀₋₁-(1-4C)alkoxy,
   —[CH₂]₀₋₁-(3-6C)cycloalkoxy,
   —[CH₂]₀₋₁—C(O)NH₂,
   —[CH₂]₀₋₁—NH₂,
   —[CH₂]₀₋₁—NH(1-2C)alkyl,
   —[CH₂]₀₋₁—C(O)(1-2C)alkyl,
   —[CH₂]₀₋₁—C(O)OH,
   —[CH₂]₀₋₁—C(O)O-(1-2C)alkyl,
   a group of the formula:

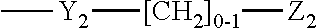

wherein Y₂ is absent, —O—, —NH—, —NMe-, —S—, —S(O)— or —S(O)₂—; and
Z₂ is (3-6C)cycloalkyl, phenyl, a 4- to 6-membered heterocyclyl or 5 or 6-membered heteroaryl;
and wherein:
any alkyl, alkoxy, cycloalkyl or —[CH₂]— moiety within a R_{c} substituent group is optionally substituted by one or more substituents selected from halo, hydroxy, cyano, amino, —C(O)OH, —C(O)NH₂, (1-2C)alkoxy, or (3-4C)cycloalkoxy; and
Z₂ is optionally substituted by one or more substituents selected from: halo, hydroxy, cyano, amino, —C(O)OH, —C(O)NH₂, (1-2C)alkoxy, (1-2C)alkyl, (3-4C)cycloalkyl, (3-4C)cycloalkoxy, —C(O)NH(1-2C)alkyl, —C(O)N[(1-2C)alkyl]₂, —NH(1-2C)alkyl, —N[(1-2C)alkyl]₂, —S(O)_{q}-(1-2C)alkyl (wherein q is 0, 1 or 2), —C(O)(1-2C)alkyl, —C(O)O-(1-2C)alkyl, —N(R_{f})C(O)-(1-2C)alkyl, —S(O)₂NH(1-2C)alkyl, —S(O)₂N[(1-2C)alkyl]₂, or —NHSO₂-(1-2C)alkyl, and wherein any (1-2C)alkoxy, (1-2C)alkyl, (3-4C)cycloalkyl or (3-4C)cycloalkoxy group is optionally substituted by one or more substituents selected from halo, cyano, hydroxy, (1-2C)alkyl, (1-2C)alkoxy or (1-2C)alkoxy-(1-2C)alkyl;
(38) R_{c} is selected from hydrogen, halo, cyano, —C(O)NH₂, (1-4C)alkyl, (3-6C)cycloalkyl
   —[CH₂]₀₋₁-(1-4C)alkoxy,
   —[CH₂]₀₋₁-(3-6C)cycloalkoxy,
   —[CH₂]₀₋₁—C(O)NH₂,
   —[CH₂]₀₋₁—NH₂,
   —[CH₂]₀₋₁—C(O)OH,
   a group of the formula:

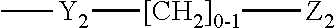

wherein Y₂ is absent, —O—, —NH— or —NMe-; and
Z₂ is (3-6C)cycloalkyl, phenyl, a 4- to 6-membered heterocyclyl or 5 or 6-membered heteroaryl;
and wherein:
any alkyl, alkoxy, cycloalkyl or —[CH₂]— moiety within a R_{c} substituent group is optionally substituted by one or more substituents selected from halo, hydroxy, cyano, amino, —C(O)OH, —C(O)NH₂, (1-2C)alkoxy, or (3-4C)cycloalkoxy; and
Z₂ is optionally substituted by one or more substituents selected from: halo, hydroxy, cyano, amino, —C(O)OH, —C(O)NH₂, (1-2C)alkoxy, (1-2C)alkyl, (3-4C)cycloalkyl, (3-4C)cycloalkoxy, —C(O)NH(1-2C)alkyl, —C(O)N[(1-2C)alkyl]₂, —NH(1-2C)alkyl, —N[(1-2C)alkyl]₂, —C(O)(1-2C)alkyl or —C(O)O-(1-2C)alkyl, and wherein any (1-2C)alkoxy, (1-2C)alkyl, (3-4C)cycloalkyl or (3-4C)cycloalkoxy group is optionally substituted by one or more substituents selected from halo, cyano, hydroxy, (1-2C)alkyl, (1-2C)alkoxy or (1-2C)alkoxy-(1-2C)alkyl;
(39) R_{c} is selected from hydrogen, halo, cyano, —C(O)NH₂, (1-4C)alkyl, (3-6C)cycloalkyl, -(1-4C)alkoxy,
   a group of the formula:

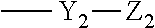

wherein Y₂ is absent, —O—, —NH— or —NMe-; and
Z₂ is (3-6C)cycloalkyl, phenyl, a 4- to 6-membered heterocyclyl or 5 or 6-membered heteroaryl;
and wherein:
any alkyl, alkoxy, cycloalkyl or —[CH₂]— moiety within a R_{c} substituent group is optionally substituted by one or more substituents selected from halo, hydroxy, cyano, amino, —C(O)OH, —C(O)NH₂, (1-2C)alkoxy, or (3-4C)cycloalkoxy; and Z₂ is optionally substituted by one or more substituents selected from: halo, hydroxy, cyano, amino, —C(O)OH, —C(O)NH₂, (1-2C)alkoxy, (1-2C)alkyl, (3-4C)cycloalkyl, (3-4C)cycloalkoxy, —C(O)NH(1-2C)alkyl, —C(O)N[(1-2C)alkyl]₂, —NH(1-2C)alkyl, —N[(1-2C)alkyl]₂, —C(O)(1-2C)alkyl or —C(O)O-(1-2C)alkyl, and wherein any (1-2C)alkoxy, (1-2C)alkyl, (3-4C)cycloalkyl or (3-4C)cycloalkoxy group is optionally substituted by one or more substituents selected from halo, cyano, hydroxy, (1-2C)alkyl, (1-2C)alkoxy or (1-2C)alkoxy-(1-2C)alkyl;
(40) R_{c} is selected from hydrogen, halo, cyano, —C(O)NH₂, (1-4C)alkyl, (3-6C)cycloalkyl, -(1-4C)alkoxy,
   a group of the formula:

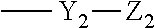

wherein Y₂ is absent or —O—; and
Z₂ is (3-4C)cycloalkyl or phenyl;
and wherein:
any alkyl, alkoxy, cycloalkyl or —[CH₂]— moiety within a R_{c} substituent group is optionally substituted by one or more substituents selected from halo, hydroxy, cyano, amino, —C(O)OH, —C(O)NH₂, (1-2C)alkoxy, or (3-4C)cycloalkoxy; and
Z₂ is optionally substituted by one or more substituents selected from: halo, hydroxy, cyano, amino, —C(O)OH, —C(O)NH₂, (1-2C)alkoxy, (1-2C)alkyl, (3-4C)cycloalkyl, (3-4C)cycloalkoxy, —C(O)NH(1-2C)alkyl, —C(O)N[(1-2C)alkyl]₂, —NH(1-2C)alkyl, —N[(1-2C)alkyl]₂, —C(O)(1-2C)alkyl or —C(O)O-(1-2C)alkyl, and wherein any (1-2C)alkoxy, (1-2C)alkyl, (3-4C)cycloalkyl or (3-4C)cycloalkoxy group is optionally substituted by one or more substituents selected from halo, cyano, hydroxy, (1-2C)alkyl, (1-2C)alkoxy or (1-2C)alkoxy-(1-2C)alkyl;
(41) R_{c} is selected from hydrogen, halo, cyano, —C(O)NH₂, (1-4C)alkyl, (3-6C)cycloalkyl, -(1-4C)alkoxy,
   a group of the formula:

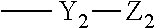

wherein Y₂ is absent or —O—; and
Z₂ is (3-4C)cycloalkyl or phenyl;
and wherein:
any alkyl, alkoxy, cycloalkyl or —[CH₂]— moiety within a Rₑ substituent group is optionally substituted by one or more substituents selected from fluoro, chloro, hydroxy, cyano, or amino; and
Z₂ is optionally substituted by one or more substituents selected from: fluoro, chloro, hydroxy, cyano, amino or (1-2C)alkyl, and wherein any (1-2C)alkyl is optionally substituted by one or more substituents selected from fluoro, chloro, cyano or hydroxy;
(42) R_{c} is selected from hydrogen, fluoro, chloro, bromo, cyano, —C(O)NH₂, methyl, —CF₃, ethyl, propyl, methoxy, —OCF₃, ethoxy, propoxy, cyclopropyl, cyclobutyl, cyclopentyl, phenyl, —O-cyclopropyl, —O-cyclobutyl, —O-cyclopentyl, -phenyl-(1-2Calkyl)-OH or -phenyl-(1-2Calkyl)-CN;
(43) R_{c} is selected from hydrogen, —CF₃, —OCF₃, cyclopropyl, phenyl, —O-cyclobutyl or -phenyl-CH₂—OH;
(44) R₁₀₀ is methyl optionally substituted by hydroxy, fluoro, chloro or bromo;
(45) R₁₀₀ is methyl optionally substituted by hydroxy or chloro;
(46) R₁₀₀ₐ is hydrogen;
(47) R₁₀₀ₐ is methyl;
(48) R₁₀₀ and R₁₀₀ₐ are linked to form a cyclopropyl ring;
(49) R₁₀₀ and R₁₀₀ₐ are linked to form a cyclobutyl ring;
(50) R₁₀₁ is hydrogen or methyl optionally substituted by hydroxy or halo;
(51) R₁₀₁ is hydrogen or methyl optionally substituted by hydroxy, fluoro, chloro or bromo;
(52) R₁₀₁ is hydrogen or methyl optionally substituted by hydroxy or chloro;
(53) R₁₀₁ₐ is hydrogen;
(54) R₁₀₁ₐ is methyl;
(55) R₁₀₁ and R₁₀₁ₐ are linked to form a cyclopropyl ring;
(56) R₁₀₁ and R₁₀₁ₐ are linked to form a cyclobutyl ring;
(57) integer a is 0, 1 or 2;
(58) integer a is 0 or 1
(59) integer a is 1
(60) Q₁ is selected from —NR₁₀₂—, —O— or —S—;
(61) Q₁ is selected from —NR₁₀₂— or —O—;
(62) Q₁ is —NR₁₀₂—;
(63) R₁₀₂ is hydrogen or methyl;
(64) R₁₀₂ is hydrogen
(65) [symbol] represents a double bond;
(66) Q₂ is N or CRₐ;
   Q₃ is N or CR_{b};
   Q₄ is N or CR_{c};
   Q₅ is N or CR_{d};
   Q₆ is N or CH;
   Rₐ, R_{b}, R_{c}, R_{d} and Rₑ are each as defined in any one of paragraphs (20) to (23), (24) to (34) and (35) to (43) above;
   with the proviso that one to three of Q₂, Q₃, Q₄, Q₅ or Q₆ is/are N;
(67) Q₂ is N or CH;
   Q₃ is N or CR_{b};
   Q₄ is N or CR_{c};
   Q₅ is N or CR_{d};
   Q₅ is N or CH;
   R_{b}, R_{c} and R_{d} are each as defined in any one of paragraphs (24) to (34) and (35) to (43) above;
   with the proviso that one to three of Q₂, Q₃, Q₄, Q₅ or Q₆ is/are N;
(68) Q₂ is N;
   Q₃ is CR_{b};
   Q₄ is CR_{c};
   Q₅ is CR_{d};
   Q₅ is CH;
   R_{b}, R_{c} and R_{d} are each as defined in any one of paragraphs (24) to (34) and (35) to (43) above;
(69) Q₂ is CH;
   Q₃ is N;
   Q₄ is CR_{c};
   Q₅ is CR_{d};
   Q₅ is CH;
   R_{c} and R_{d} are each as defined in any one of paragraphs (24) to (34) and (35) to (43) above;
   with the proviso that one to three of Q₂, Q₃, Q₄, Q₅ or Q₆ is/are N;
(70) Q₂ is CH;
   Q₃ is CR_{b};
   Q₄ is CR_{c};
   Q₅ is N;
   Q₆ is CH;
   R_{b} and R_{c} are each as defined in any one of paragraphs (24) to (34) and (35) to (43) above;
(71) Q₂ is CH;
   Q₃ is CR_{b};
   Q₄ is CR_{c};
   Q₅ is CR_{d};
   Q₆ is N;
   R_{b}, R_{c} and R_{d} are each as defined in any one of paragraphs (24) to (34) and (35) to (43) above;
(72) Q₇ is N or CR_{f};
   Q₈ is N or CR_{f};
   Q₉ is N or CR_{f};
   Q₁₀ is N or CR_{f};
   with the proviso that one or two of Q₇, Q₈, Q₉ or Q₁₀ is/are N;
   each R_{f} present is independently selected from hydrogen, methyl fluoro, chloro or bromo;
(73) Q₇ is N or CR_{f};
   Q₈ is N or CR_{f};
   Q₉ is N or CR_{f};
   Q₁₀ is N or CR_{f};
   with the proviso that one or two of Q₇, Q₈, Q₉ or Q₁₀ is/are N;
   each R_{f} present is hydrogen;
(74) Q₇ is N;
   Q₈ is CH;
   Q₉ is CH;
   Q₁₀ is CH;
(75) Ring A is a five-membered heteroaryl ring comprising one, two or three heteroatoms selected from N, O or S, optionally substituted by one R_{b} (as defined herein) and/or one or two R_{c} substituents (as defined herein);
(76) Ring A is a five-membered heteroaryl ring comprising one or two heteroatoms selected from N, O or S, optionally substituted by one R_{b} (as defined in any one of paragraphs (24) to (34) above) and/or one or two R_{c} substituents (as defined in any one of paragraphs (35) to (43) above);
(77) Ring A is a five-membered heteroaryl ring comprising one or two heteroatoms selected from N or O, optionally substituted by one R_{b} (as defined in any one of paragraphs (30) to (34) above) and/or one or two R_{c} substituents (as defined in any one of paragraphs (40) to (43) above);
(78) Ring A is a five-membered heteroaryl ring comprising two heteroatoms selected from N or O, optionally substituted by one R_{b} (as defined in paragraph (34) above) and/or one or two R_{c} substituents (as defined in paragraph (43) above);
(79) Ring A is a pyrazole ring substituted with one, two or three groups independently selected from phenyl, methyl, chloro;
(80) Ring A is a pyrazole ring substituted with one phenyl group and one chloro group;
(81) R_{R} is selected from one of formulae Im or In shown below:

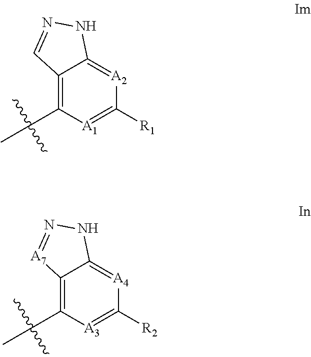

wherein:
[symbol] denotes the point of attachment;
(82) R_{R} is formula Im shown below:

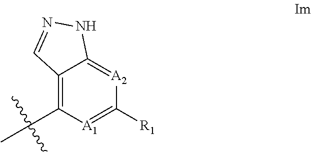

wherein:
[symbol] denotes the point of attachment;
(83) A₁ and A₂ are both CH;
(84) one of A₁ and A₂ is N and the other is CH;
(85) R₁ is a 5- or 6-membered heteroaryl ring comprising one, two, three or four heteroatoms which is optionally substituted on any available carbon atom by one or more R_{1A} substituent groups and on any available nitrogen atom by one or more R_{1B} substituent groups;
(86) R₁ is a 5- or 6-membered heteroaryl ring comprising one, two, three or four heteroatoms selected from N, O or S which is optionally substituted on any available carbon atom by one or more R_{1A} substituent groups and on any available nitrogen atom by one or more R_{1B} substituent groups;
(87) R₁ is a 5- or 6-membered heteroaryl ring comprising one, two, or three heteroatoms selected from N, O or S which is optionally substituted on any available carbon atom by one or more R_{1A} substituent groups and on any available nitrogen atom by one or more R_{1B} substituent groups;
(88) R₁ is selected from:

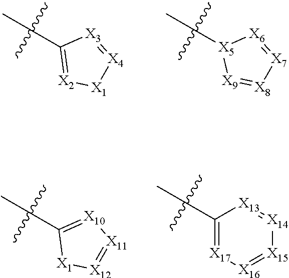

wherein:
[symbol] denotes the point of attachment;
X₁ is NH, NR_{1B}, O or S;
X₅ is N;
X₂, X₃, X₄, X₆, X₇, X₈, X₉, X₁₀, X₁₁, X₁₂, X₁₃, X₁₄, X₁₅, X₁₆ or X₁₇ are selected from CH, CR_{1A}, N or N⁺—O⁻;
and R_{1A} and R_{1B} are both as defined herein;
(89) R₁ is selected from:

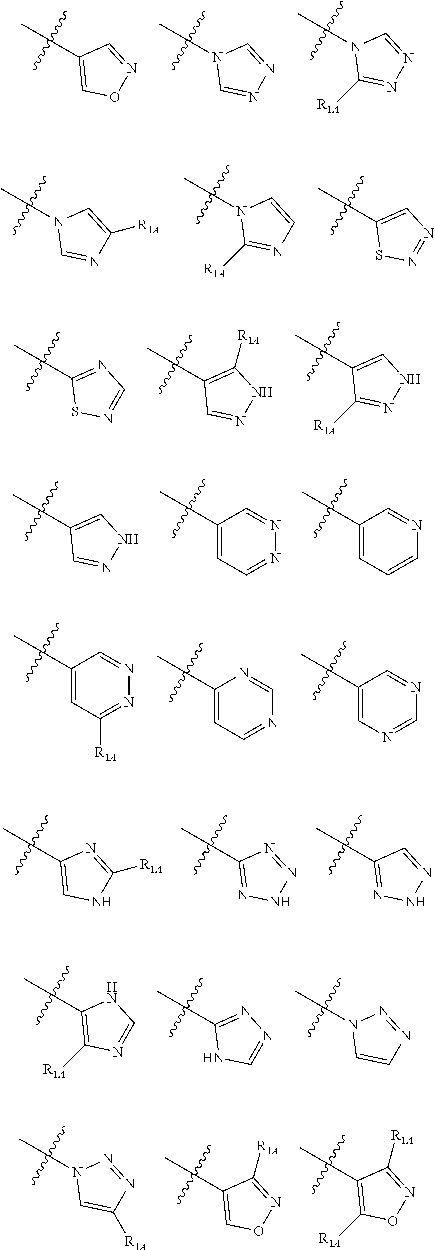

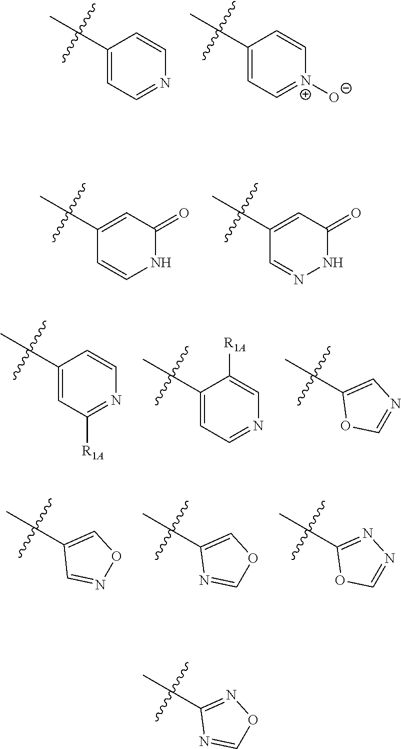

wherein R_{1A} is as defined herein;
(90) R₁ is selected from:

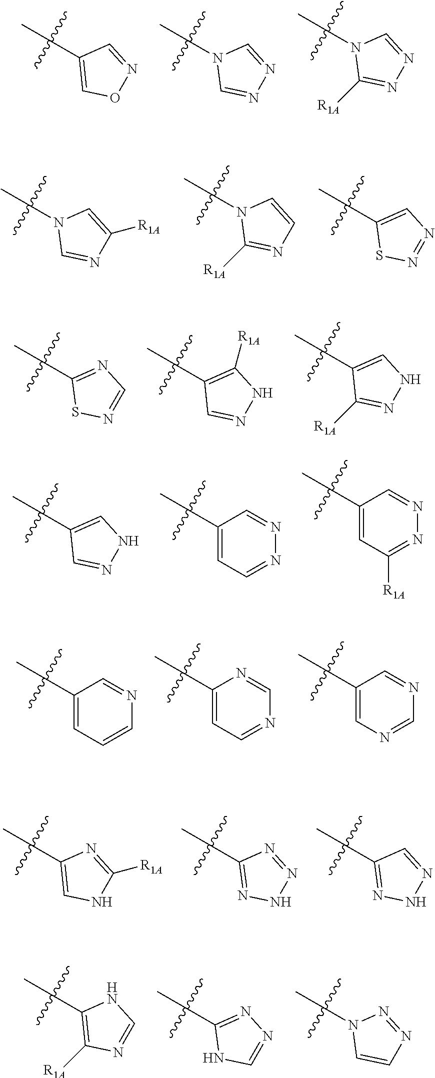

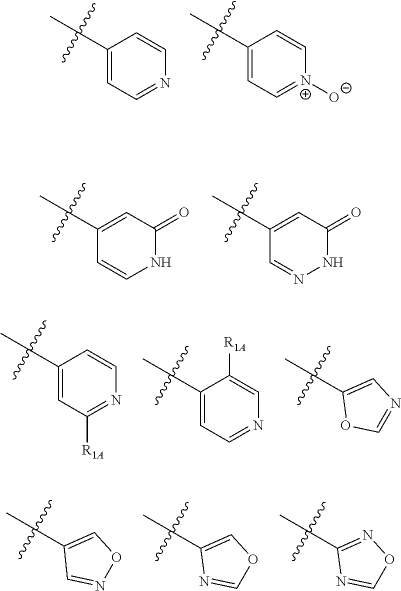

wherein R_{1A} is as defined herein;
(91) R₁ is selected from:

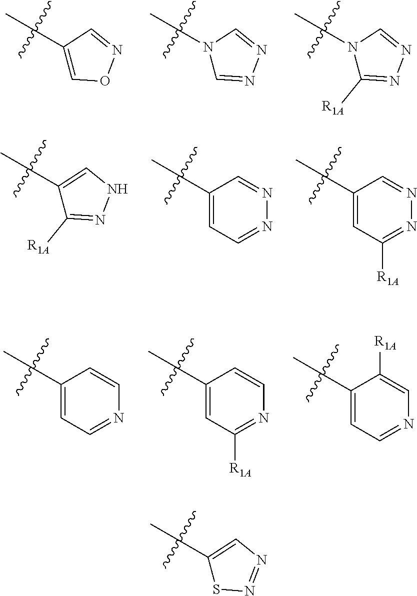

wherein R_{1A} is as defined herein;
(92) X₁ is NH, NMe, O or S;
(93) X₁ is NH, O or S;
(94) X₁ is NH;
(95) X₁ is 0;
(96) X₁ is S;
(97) X₅ is N;
(98) X₂, X₃ and X₄ are each independently selected from CH, CR_{1A} or N, wherein R_{1A} is as defined herein;
(99) X₂, X₃ and X₄ are each independently selected from CH, C-(1-2C)alkyl, C—CN, C—OH, C—NH₂, C—O-(1-2C)alkyl, C-halo or N;
(100) X₂, X₃ and X₄ are each independently selected from CH, C—CH₃, C—CH₂CH₃, C—CN, C—OH, C—NH₂, CO—CH₃, C—O—CH₂CH₃, C—F, C—Cl, C—Br or N;
(101) X₂, X₃ and X₄ are each independently selected from CH, C—CH₃, C—CN, C—O—CH₃, or N;
(102) X₂, X₃ and X₄ are all N;
(103) One or two of X₂, X₃ and X₄ are N and the others are each independently selected from CH, C—CH₃, C—CN, or C—O—CH₃;
(104) One of X₂, X₃ and X₄ is N and the others are each independently selected from CH, C—CH₃, C—CN or C—O—CH₃;
(105) Two of X₂, X₃ and X₄ is N and the other is CH;
(106) X₆, X₇, X₈ and X₉ are each independently selected from CH, CR_{1A} or N, wherein R_{1A} is as defined herein;
(107) X₆, X₇, X₈ and X₉ are each independently selected from CH, C-(1-2C)alkyl, C—CN, C—OH, C—NH₂, C—O-(1-2C)alkyl, C-halo or N;
(108) X₆, X₇, X₈ and X₉ are each independently selected from CH, C—CH₃, C—CH₂CH₃, C—CN, C—OH, C—NH₂, CO—CH₃, C—O—CH₂CH₃, C—F, C—Cl, C—Br or N;
(109) X₆, X₇, X₈ and X₉ are each independently selected from CH, C—CH₃, C—CN, C—OH or N;
(110) One or two of X₆, X₇, X₈ and X₉ are N and the others are each independently selected from CH, C—CH₃, C—CN or C—OH;
(111) Two of X₆, X₇, X₈ and X₉ are N and the others are each independently selected from CH, C—CH₃, C—CN or C—OH;
(112) Two of X₆, X₇, X₈ and X₉ are N and the others are both CH;
(113) X₁₀, X₁₁ and X₁₂ are each independently selected from CH, CR_{1A} or N, wherein R_{1A} is as defined herein;
(114) X₁₀, X₁₁ and X₁₂ are each independently selected from CH, C-(1-2C)alkyl, C—CN, C—OH, C—NH₂, C—O-(1-2C)alkyl, C-halo or N;
(115) X₁₀, X₁₁ and X₁₂ are each independently selected from CH, C—CH₃, C—CH₂CH₃, C—CN, C—OH, C—NH₂, CO—CH₃, C—O—CH₂CH₃, C—F, C—Cl, C—Br or N;
(116) One or two of X₁₀, X₁₁ and X₁₂ N and the others are each independently selected from CH or C—CH₃;
(117) Two of X₁₀, X₁₁ and X₁₂ N and the other is CH;
(118) X₁₃, X₁₄, X₁₅, X₁₆ and X₁₇ are each independently selected from CH, CR_{1A} or N, wherein R_{1A} is as defined herein;
(119) X₁₃, X₁₄, X₁₅, X₁₆ and X₁₇ are each independently selected from CH, C-(1-2C)alkyl, C—CN, C—OH, C(O), C—NH₂, C—O-(1-2C)alkyl, C-halo, N or N-oxide;
(120) X₁₃, X₁₄, X₁₅, X₁₆ and X₁₇ are each independently selected from CH, C—CH₃, C—CH₂CH₃, C—CN, C—OH, C(O), C—NH₂, C—O—CH₃, C—O—CH₂CH₃, C—F, C—Cl, C-Br, N or N-oxide;
(121) One or two of X₁₃, X₁₄, X₁₅, X₁₆ and X₁₇ are N and the others are each independently selected from CH, C—CH₃, C—CH₂CH₃, C—CN, C—OH, C(O), C—NH₂, C—O—CH₃, C—O—CH₂CH₃, C—F, C—Cl, C-Br, N or N-oxide;
(122) One or two of X₁₃, X₁₄, X₁₅, X₁₆ and X₁₇ are N and the others are each independently selected from CH, C—CH₃, C—CN, C—OH, C(O), C—NH₂, CO—CH₃, C—F, C—Cl, C—Br or N-oxide;
(123) One of X₁₃, X₁₄, X₁₅, X₁₆ and X₁₇ are N and the others are each independently selected from CH, C—CH₃, C—CN, C(O), C—NH₂, CO—CH₃, C—F or C—Cl;
(124) Two of X₁₃, X₁₄, X₁₅, X₁₆ and X₁₇ are N and the others are each independently selected from CH, C—CN, C—OH, or C—NH₂;
(125) Two of X₁₃, X₁₄, X₁₅, X₁₆ and X₁₇ are N, one is C—OH or C—NH₂, and the others are CH;
(126) Two of X₁₃, X₁₄, X₁₅, X₁₆ and X₁₇ are N and the others are CH;
(127) each R_{1A} group present is selected from hydroxy, cyano, amino, halo, —C(O)OH, —C(O)NH₂, (1-2C)alkoxy, (1-2C)alkyl, (3-4C)cycloalkyl, (3-4C)cycloalkoxy, —C(O)NH(1-2C)alkyl, —C(O)N[(1-2C)alkyl]₂, —NH(1-2C)alkyl, —N[(1-2C)alkyl]₂, or —C(O)(1-2C)alkyl, —C(O)O-(1-2C)alkyl; and wherein any (1-2C)alkoxy, (1-2C)alkyl, (3-4C)cycloalkyl or (3-4C)cycloalkoxy group is optionally substituted by one or more substituents selected from halo, cyano, hydroxy, (1-2C)alkyl, (1-2C)alkoxy or (1-2C)alkoxy-(1-2C)alkyl;
(128) each R_{1A} group present is selected from hydroxy, cyano, amino, halo, —C(O)OH, —C(O)NH₂, (1-2C)alkoxy, (1-2C)alkyl, (3-4C)cycloalkyl, (3-4C)cycloalkoxy, —C(O)NH(Me), —C(O)N[Me]₂, —NH(Me), —N(Me)₂, or —C(O)(Me), —C(O)O-(Me); and wherein any (1-2C)alkoxy, (1-2C)alkyl, methyl, (3-4C)cycloalkyl or (3-4C)cycloalkoxy group is optionally substituted by one or more substituents selected from halo, cyano, hydroxy, (1-2C)alkyl, (1-2C)alkoxy or (1-2C)alkoxy-(1-2C)alkyl;
(129) each R_{1A} group present is selected from hydroxy, cyano, amino, halo, —C(O)OH, —C(O)NH₂, (1-2C)alkoxy, (1-2C)alkyl, (3-4C)cycloalkyl, (3-4C)cycloalkoxy, —C(O)NH(Me), —C(O)N[Me]₂, —NH(Me), —N(Me)₂, or —C(O)(Me), —C(O)O-(Me); and wherein any (1-2C)alkoxy, (1-2C)alkyl, methyl, (3-4C)cycloalkyl or (3-4C)cycloalkoxy group is optionally substituted by one or more substituents selected from halo, cyano, hydroxy, (1-2C)alkyl, (1-2C)alkoxy or (1-2C)alkoxy-(1-2C)alkyl;
(130) each R_{1A} group present is selected from hydroxy, cyano, amino, chloro, fluoro, —C(O)OH, —C(O)NH₂, (1-2C)alkoxy, (1-2C)alkyl; and wherein any (1-2C)alkoxy, (1-2C)alkyl group is optionally substituted by one or more substituents selected from halo, cyano, hydroxy, (1-2C)alkyl, (1-2C)alkoxy or (1-2C)alkoxy-(1-2C)alkyl;
(131) each R_{1A} group present is selected from —OH, —CN, NH₂, —F, —Cl, ═O, —OCH₃, or —CH₃;
(132) each R_{1A} group present is selected from —OH, —CN, NH₂, —F, ═O, —OCH₃, or —CH₃;
(133) each R_{1B} group is methyl, ethyl, cyclopropyl or cyclobutyl;
(134) each R_{1B} group is methyl;
(135) A₃ is selected from CH;
(136) A₃ is selected from CR₃;
(137) A₃ is selected from N;
(138) A₄ is selected from CH;
(139) A₄ is selected from CR₄;
(140) A₄ is selected from N;
(141) A₅ and A₆ are both C;
(142) one of A₅ and A₆ is N and the other is C;
(143) A₇ is selected from CH;
(144) A₇ is selected from CR₇;
(145) A₇ is selected from N;
(146) R₂ is selected from:
(i) a group R₁ defined above;
(ii) hydrogen, halo, cyano, (1-4C)alkyl, (1-4C)cyanoalkyl, (1-4C)hydroxyalkyl, (1-4C)haloalkyl, (1-4C)aminoalkyl,
   —[CH₂]₀₋₂-(1-4C)alkoxy,
   —[CH₂]₀₋₂—C(O)NH₂,
   —[CH₂]₀₋₂—C(O)NH(1-4C)alkyl,
   —[CH₂]₀₋₂—C(O)N[(1-4C)alkyl]₂,
   —[CH₂]₀₋₂—NH(1-4C)alkyl,
   —[CH₂]₀₋₂—N[(1-4C)alkyl]₂,
   —[CH₂]₀₋₂—S(O)_{q}-(1-4C)alkyl (wherein q is 0, 1 or 2),
   —[CH₂]₀₋₂—C(O)(1-4C)alkyl,
   —[CH₂]₀₋₂—C(O)OH,
   —[CH₂]₀₋₂—C(O)O-(1-4C)alkyl,
   —[CH₂]₀₋₂—N(R₂ₐ)C(O)-(1-4C)alkyl (wherein R₂ₐ is hydrogen or methyl),
   —[CH₂]₀₋₂—S(O)₂NH(1-4C)alkyl,
   —[CH₂]₀₋₂—S(O)₂N[(1-4C)alkyl]₂,
   —[CH₂]₀₋₂—N(R_{2b})SO₂-(1-4C)alkyl (wherein R_{2b} is hydrogen or methyl),
   a 4- to 7-membered heterocyclyl,
   (3-6C)cycloalkyl,
   (3-6C)cycloalkyl(1-2C)alkyl,
   phenyl, or
   phenyl(1-2C)alkyl;
   and wherein any cycloalkyl, heterocyclyl or phenyl group present is optionally substituted by one or more hydroxy, cyano, amino, halo, —C(O)OH, —C(O)NH₂, (1-2C)alkoxy, (1-2C)alkyl, —C(O)NH(1-2C)alkyl, —C(O)N[(1-2C)alkyl]₂, —NH(1-2C)alkyl, —N[(1-2C)alkyl]₂, —S(O)_{q}-(1-2C)alkyl (wherein q is 0, 1 or 2), —C(O)(1-2C)alkyl, —C(O)O-(1-2C)alkyl, —N(R_{f})C(O)-(1-2C)alkyl (wherein R_{f} is hydrogen or methyl), —S(O)₂NH(1-2C)alkyl, —S(O)₂N[(1-2C)alkyl]₂, or —NHSO₂-(1-2C)alkyl;
(147) R₂ is selected from:
(i) a group R₁ defined above;
(ii) hydrogen, halo, cyano, (1-4C)alkyl, (1-4C)cyanoalkyl, (1-4C)hydroxyalkyl, (1-4C)haloalkyl, (1-4C)aminoalkyl,
   —[CH₂]₀₋₂-(1-4C)alkoxy,
   —[CH₂]₀₋₂—C(O)NH₂,
   —[CH₂]₀₋₂—C(O)NH(1-2C)alkyl,
   —[CH₂]₀₋₂—C(O)N[(1-4C)alkyl]₂,
   —[CH₂]₀₋₂—NH(1-2C)alkyl,
   —[CH₂]₀₋₂—N[(1-2C)alkyl]₂,
   —[CH₂]₀₋₂—S(O)_{q}-(1-2C)alkyl (wherein q is 0, 1 or 2),
   —[CH₂]₀₋₂—C(O)(1-2C)alkyl,
   —[CH₂]₀₋₂—C(O)OH,
   —[CH₂]₀₋₂—C(O)O-(1-2C)alkyl,
   —[CH₂]₀₋₂—N(H)C(O)-(1-2C)alkyl,
   —[CH₂]₀₋₂—S(O)₂NH(1-2C)alkyl,
   —[CH₂]₀₋₂—S(O)₂N[(1-2C)alkyl]₂,
   —[CH₂]₀₋₂—N(H)SO₂-(1-2C)alkyl,
   a 4- to 7-membered heterocyclyl,
   (3-6C)cycloalkyl,
   (3-6C)cycloalkyl(1-2C)alkyl,
   phenyl, or
   phenyl(1-2C)alkyl;
   and wherein any cycloalkyl, heterocyclyl or phenyl group present is optionally substituted by one or more hydroxy, cyano, amino, halo, —C(O)OH, —C(O)NH₂, (1-2C)alkoxy, (1-2C)alkyl, —C(O)NH(1-2C)alkyl, —C(O)N[(1-2C)alkyl]₂, —NH(1-2C)alkyl, —N[(1-2C)alkyl]₂, —S(O)_{q}-(1-2C)alkyl (wherein q is 0, 1 or 2), —C(O)(1-2C)alkyl, —C(O)O-(1-2C)alkyl, —N(R_{f})C(O)-(1-2C)alkyl (wherein R_{f} is hydrogen or methyl), —S(O)₂NH(1-2C)alkyl, —S(O)₂N[(1-2C)alkyl]₂, or —NHSO₂-(1-2C)alkyl;
(148) R₂ is selected from:
(i) a group R₁ defined above;
(ii) hydrogen, halo, cyano, (1-4C)alkyl, (1-4C)cyanoalkyl, (1-4C)hydroxyalkyl, (1-4C)haloalkyl, (1-4C)aminoalkyl,
   —[CH₂]₀₋₁-(1-4C)alkoxy,
   —[CH₂]₀₋₁—C(O)NH₂,
   —[CH₂]₀₋₁—C(O)NH(1-2C)alkyl,
   —[CH₂]₀₋₁—C(O)N[(1-4C)alkyl]₂,
   —[CH₂]₀₋₁—NH(1-2C)alkyl,
   —[CH₂]₀₋₁—N[(1-2C)alkyl]₂,
   —[CH₂]₀₋₁—S(O)_{q}-(1-2C)alkyl (wherein q is 0, 1 or 2),
   —[CH₂]₀₋₁—C(O)(1-2C)alkyl,
   —[CH₂]₀₋₂—C(O)OH,
   —[CH₂]₀₋₁—C(O)O-(1-2C)alkyl,
   —[CH₂]₀₋₁—N(H)C(O)-(1-2C)alkyl,
   —[CH₂]₀₋₁—S(O)₂NH(1-2C)alkyl,
   —[CH₂]₀₋₁—S(O)₂N[(1-2C)alkyl]₂,
   —[CH₂]₀₋₁—N(H)SO₂-(1-2C)alkyl,
   a 4- to 7-membered heterocyclyl,
   (3-6C)cycloalkyl,
   (3-6C)cycloalkyl(1-2C)alkyl,
   phenyl, or
   phenyl(1-2C)alkyl;
   and wherein any cycloalkyl, heterocyclyl or phenyl group present is optionally substituted by one or more hydroxy, cyano, amino, halo, —C(O)OH, —C(O)NH₂, (1-2C)alkoxy, (1-2C)alkyl, —C(O)NH(1-2C)alkyl, —C(O)N[(1-2C)alkyl]₂, —NH(1-2C)alkyl, —N[(1-2C)alkyl]₂, —S(O)_{q}-(1-2C)alkyl (wherein q is 0, 1 or 2), —C(O)(1-2C)alkyl, —C(O)O-(1-2C)alkyl, —N(R_{f})C(O)-(1-2C)alkyl (wherein R_{f} is hydrogen or methyl), —S(O)₂NH(1-2C)alkyl, —S(O)₂N[(1-2C)alkyl]₂, or —NHSO₂-(1-2C)alkyl;
(149) R₂ is selected from:
(i) a group R₁ defined above;
(ii) hydrogen, halo, cyano, (1-4C)alkyl, (1-4C)cyanoalkyl, (1-4C)hydroxyalkyl, (1-4C)haloalkyl, (1-4C)aminoalkyl,
   —[CH₂]₀₋₁-(1-4C)alkoxy,
   —[CH₂]₀₋₁—C(O)NH₂,
   —[CH₂]₀₋₁—C(O)NH(1-2C)alkyl,
   —[CH₂]₀₋₁—C(O)N[(1-4C)alkyl]₂,
   —[CH₂]₀₋₁—C(O)(1-2C)alkyl,
   —[CH₂]₀₋₂—C(O)OH,
   —[CH₂]₀₋₁—C(O)O-(1-2C)alkyl,
   —[CH₂]₀₋₁—N(H)C(O)-(1-2C)alkyl,
   a 4- to 7-membered heterocyclyl,
   (3-6C)cycloalkyl,
   (3-6C)cycloalkyl(1-2C)alkyl,
   phenyl, or
   phenyl(1-2C)alkyl;
   and wherein any cycloalkyl, heterocyclyl or phenyl group present is optionally substituted by one or more hydroxy, cyano, amino, halo, —C(O)OH, —C(O)NH₂, methoxy, methyl, —C(O)NH(Me), —C(O)N(Me)₂, —NH(Me), —N(Me)₂, —S(O)_{q}-(Me) (wherein q is 0, 1 or 2), —C(O)(Me), —C(O)O-(Me), —N(H)C(O)-(Me), —S(O)₂NH(Me), —S(O)₂N(Me)₂, or —NHSO₂-(Me);
(150) R₂ is selected from:
(i) a group R₁ defined above;
(ii) hydrogen, halo, cyano, (1-2C)alkyl, (1-2C)cyanoalkyl, (3-4C)cyanoalkyl, (1-2C)hydroxyalkyl, (1-2C)haloalkyl, (1-2C)aminoalkyl,
   —[CH₂]₀₋₁-(1-2C)alkoxy,
   —[CH₂]₀₋₁—C(O)NH₂,
   —[CH₂]₀₋₁—C(O)NH(1-2C)alkyl,
   —[CH₂]₀₋₁—C(O)N[(1-2C)alkyl]₂,
   —[CH₂]₀₋₁—C(O)(1-2C)alkyl,
   —[CH₂]₀₋₂—C(O)OH,
   —[CH₂]₀₋₁—C(O)O-(1-2C)alkyl,
   —[CH₂]₀₋₁—N(H)C(O)-(1-2C)alkyl,
   a 4- to 6-membered heterocyclyl,
   (4-6C)cycloalkyl,
   (4-6C)cycloalkyl(1-2C)alkyl,
   phenyl, or
   phenyl(1-2C)alkyl;
   and wherein any cycloalkyl, heterocyclyl or phenyl group present is optionally substituted by one or more hydroxy, cyano, amino, halo, —C(O)OH, —C(O)NH₂, methoxy, methyl, —C(O)NH(Me), —C(O)N(Me)₂, —NH(Me), —N(Me)₂, —S(O)_{q}-(Me) (wherein q is 0, 1 or 2), —C(O)(Me), —C(O)O-(Me), —N(H)C(O)-(Me), —S(O)₂NH(Me), —S(O)₂N(Me)₂, or —NHSO₂-(Me);
(151) R₂ is selected from:
(i) a group R₁ defined above;
(ii) hydrogen, halo, cyano, (1-2C)alkyl, (1-2C)cyanoalkyl, (3-4C)cyanoalkyl, (1-2C)hydroxyalkyl, (1-2C)haloalkyl, (1-2C)aminoalkyl,
   —[CH₂]₀₋₁-(1-2C)alkoxy,
   —[CH₂]₀₋₂—C(O)NH₂,
   —[CH₂]₀₋₁—C(O)N[(1-2C)alkyl]₂,
   —[CH₂]₀₋₁—C(O)OH,
   a 4- to 6-membered heterocyclyl,
   (4-6C)cycloalkyl,
   phenyl,
   and wherein any cycloalkyl, heterocyclyl or phenyl group present is optionally substituted by one or more hydroxy, cyano, amino, halo, —C(O)OH, —C(O)NH₂, methoxy, methyl, —C(O)NH(Me), —C(O)N(Me)₂, —NH(Me), —N(Me)₂, —S(O)_{q}-(Me) (wherein q is 0, 1 or 2), —C(O)(Me), —C(O)O-(Me), —N(H)C(O)-(Me), —S(O)₂NH(Me), —S(O)₂N(Me)₂, or —NHSO₂-(Me);
(152) R₂ is selected from:
(i) a group R₁ defined above;
(ii) hydrogen, chloro, fluoro, cyano, methyl, ethyl, —CH₂CN, —C(CH₃)₂CN, —CH₂OH, (1-2C)haloalkyl, —CH₂NH₂, —OCH₃, —C(O)NH₂, —C(O)N(Me)₂, CH₂CH₂—C(O)OH, —C(O)OH, -morpholino, -oxetane, -tetrahydrofuran or -tetrahyrdopyran;
(153) R₂ is selected from:
(i) a group R₁ defined above;
(ii) hydrogen, cyano, —C(O)NH₂, —C(O)N(Me)₂, CH₂CH₂—C(O)OH, —C(O)OH, -morpholino, -oxetane, -tetrahydrofuran, -tetrahyrdopyran, —CH₂CN or —C(CH₃)₂CN;
(154) R₃ is selected from fluoro, chloro, methyl or methoxy;
(155) R₃ is chloro;
(156) R₄ is selected from fluoro, chloro, methyl or methoxy;
(157) R₄ is selected from fluoro, chloro or methyl;
(158) R₇ is selected from methyl or —NH(Me);
(159) R₇ is —NH(Me);
(160) A₈ is selected from CH, CR₈, O, S or N;
(161) A₉ is selected from CH, CR₉, O, S or N;
(162) A₁₀ is selected from CH, CR₁₀, O, S, NH or N;
(163) R₈, R₉ and R₁₀ are selected from methyl, amino or —NH(Me).

In a particular group of compounds of the invention, when L is a group of formula II defined herein, either:
(i) X_{A} is not NH, when L_{A} is an unsubstituted (4C)alkylene (butylene), X_{B} is —O—, L_{B} is an unsubstituted (2C)alkylene (ethylene), and X_{C} is —O— or —NH—;
(ii) L_{A} is not an unsubstituted (4C)alkylene (butylene), when X_{A} is NH, X_{B} is —O—, L_{B} is an unsubstituted (2C)alkylene (ethylene), and X_{C} is —O— or —NH—;
(iii) X_{B} is not —O—, when X_{A} is NH, L_{A} is an unsubstituted (4C)alkylene (butylene), L_{B} is an unsubstituted (2C)alkylene (ethylene), and X_{C} is —O— or —NH—;
(iv) L_{B} is not an unsubstituted (2C)alkylene (ethylene), when X_{A} is NH, L_{A} is an unsubstituted (4C)alkylene (butylene), X_{B} is —O—, and X_{C} is —O— or —NH—;
(v) X_{c} is not —O— or —NH—, when X_{A} is NH, L_{A} is an unsubstituted (4C)alkylene (butylene), X_{B} is —O—, and L_{B} is an unsubstituted (2C)alkylene (ethylene).

In a further group of compounds of the invention, when L is a group of formula ∥ defined herein, L_{A} is suitably selected from:
(i) a (4C)alkylene substituted by one or more R_{LA} substituent groups;
(ii) a (2-3C)alkylene or a (5C)alkylene optionally substituted by one or more R_{LA} substituent groups; or
(iii) a (0-3C)alkylene-(3-6C)cycloalkylene-(0-3C)alkylene- group optionally substituted by one or more RLA substituent groups.

In a further group of compounds of the invention, when L is a group of formula ∥ defined herein, L_{A} is suitably selected from:
(i) a (4C)alkylene substituted by one or more R_{LA} substituent groups;
(ii) a (2-3C)alkylene or a (5C)alkylene optionally substituted by one or more R_{LA} substituent groups.

Suitably, in any of the definitions of formula I set out herein, at least one of Rₐ, R_{b}, R_{c}, R_{d} or Rₑ is a non-hydrogen substituent. By “non-hydrogen substituent” we mean a substituent selected from any one of the options defined herein for Rₐ, R_{b}, R_{c}, R_{d} or Rₑ other than hydrogen. More suitably, one to four of Rₐ, R_{b}, R_{c}, R_{d} or Rₑ is/are a non-hydrogen substituent(s). Most suitably, one to three of Rₐ, R_{b}, R_{c}, R_{d} or Rₑ is/are a non-hydrogen substituent(s).

Suitably, in any of the definitions of formula I set out herein, up to four of Rₐ, R_{b}, R_{c}, R_{d} or Rₑ are hydrogen and the remainder are non-hydrogen substituents (i.e. selected from any one of the options set out herein for Rₐ, R_{b}, R_{c}, R_{d} or Rₑ other than hydrogen). More suitably, two to four of Rₐ, R_{b}, R_{c}, R_{d} or Rₑ are hydrogen and the remainder are non-hydrogen substituents.

In a particular group of compounds of formula I, if Rₑ is a group of the formula —Y₂—[CH₂]₀₋₃—Z₂, then R_{b} and R_{d} cannot be a group of the formula —Y₁—[CH₂]₀₋₃—Z₁.

In a further group of compounds of formula I, if one or both of R_{b} and R_{d} is a group of the formula —Y₁—[CH₂]₀₋₃—Z₁ as defined herein, then R_{c} cannot be a group of the formula —Y₂—[CH₂]₀₋₃—Z₂.

In a particular group of compounds of formula I:
if R_{c} is a group of the formula —Y₂—[CH₂]₀₋₃—Z₂ then R_{b} and R_{d} cannot be a group of the formula —Y₁—[CH₂]₀₋₃—Z; and/or
if one or both of R_{b} and R_{d} is a group of the formula —Y₁—[CH₂]₀₋₃—Z₁ as defined herein, then R_{c} cannot be a group of the formula —Y₂—[CH₂]₀₋₃—Z₂.

In another particular group of compounds of formula I:
I. if R_{c} is a group of the formula —Y₂—[CH₂]₀₋₃—Z₂ then R_{b} and R_{d} cannot be a group of the formula —Y₁—[CH₂]₀₋₃—Z₁; and
II. if one of R_{b} and R_{d} is a group of the formula —Y₁—[CH₂]₀₋₃—Z₁ as defined herein, then the other cannot be a group of the formula —Y₁—[CH₂]₀₋₃—Z₁ and R_{c} cannot be a group of the formula —Y₂—[CH₂]₀₋₃—Z₂.

Suitably, in any of the definitions of formula I set out herein, a heteroaryl is a 5- or 6-membered heteroaryl ring comprising one, two or three heteroatoms selected from N, O or S, unless specified otherwise.

Suitably, in any of the definitions of formula I set out herein, a heterocyclyl group is a 4-, 5- or 6-membered heterocyclyl ring comprising one, two or three heteroatoms selected from N, O or S, unless specified otherwise. Most suitably, a heterocyclyl group is a 4-, 5- or 6-membered ring comprising one or two heteroatoms selected from N, O or S [e.g. morpholinyl (e.g. 4-morpholinyl), piperidinyl, piperazinyl or pyrrolidinyl].

Suitably, in any of the definitions of formula I set out herein, L is as defined in formula I above or as defined in any one of paragraphs (1) to (13) above. More suitably, L is as defined in any one of paragraphs (4) to (13) above. Even more suitably, L is as defined in any one of paragraphs (6) to (13) above. Yet even more suitably, L is as defined in any one of paragraphs (8) to (13) above. Yet still even more suitably, L is as defined in any one of paragraphs (10) to (13) above. Most suitably, L is as defined in any one of paragraphs (12) or (13) above.

Suitably, in any of the definitions of formula I set out herein, R_{L} is as defined in any one of paragraphs (14) to (19) above. More suitably, R_{L} is as defined in any one of paragraphs (15) to (19) above. Even more suitably, R_{L} is as defined in any one of paragraphs (16) to (19) above. Yet even more suitable, R_{L} is as defined in any one of paragraphs (17), (18) or (19) above. Most suitably, R_{L} is as defined in any one of paragraphs (18) or (19) above.

Suitably, in any of the definitions of formula I set out herein, Rₐ and Rₑ are as defined in any one of paragraphs (20) to (23) above. More suitably, Rₐ and Rₑ are as defined in any one of paragraphs (21), (22) or (23) above. Even more suitably, Rₐ and Rₑ are as defined in any one of paragraphs (22) or (23) above. Most suitably, Rₐ and Rₑ are as defined in paragraph (23) above.

In a particular group of compounds of formula I, Rₐ and Rₑ are as defined in paragraph (23) above, and L and R_{L}, and any group associated therewith, are each as defined in formula I above.

Suitably, in any of the definitions of formula I set out herein, R_{b} and R_{d} are as defined in any one of paragraphs (24) to (34) above. More suitably, R_{b} and R_{d} are as defined in any one of paragraphs (26) to (34) above. Even more suitably, R_{b} and R_{d} are as defined in any one of paragraphs (28) to (34) above. Yet more suitably, R_{b} and R_{d} are as defined in any one of paragraphs (30) to (34) above. Yet even more suitably, R_{b} and R_{d} are as defined in any one of paragraphs (31), (32), (33) or (34) above. Yet still even more suitably, R_{b} and R_{d} are as defined in any one of paragraphs (32), (33) or (34) above. Most suitably, R_{b} and R_{d} are as defined in any one of paragraphs (33) or (34) above.

In a particular group of compounds of formula I, R_{b} and R_{d} are as defined in paragraph (24) above, and L and R_{L}, and any group associated therewith, are each as defined in formula I above.

In a particular group of compounds of formula I, R_{b} and R_{d} are as defined in paragraph (26) above, and L and R_{L}, and any group associated therewith, are each as defined in formula I above.

In a particular group of compounds of formula I, R_{b} and R_{d} are as defined in paragraph (28) above, and L and R_{L}, and any group associated therewith, are each as defined in formula I above.

In a particular group of compounds of formula I, R_{b} and R_{d} are as defined in paragraph (30) above, and L and R_{L}, and any group associated therewith, are each as defined in formula I above.

In a particular group of compounds of formula I, R_{b} and R_{d} are as defined in paragraph (31) above, and L and R_{L}, and any group associated therewith, are each as defined in formula I above.

In a particular group of compounds of formula I, R_{b} and R_{d} are as defined in paragraph (32) above, and L and R_{L}, and any group associated therewith, are each as defined in formula I above.

In a particular group of compounds of formula I, R_{b} and R_{d} are as defined in paragraph (33) above, and L and R_{L}, and any group associated therewith, are each as defined in formula I above.

In a particular group of compounds of formula I, R_{b} and R_{d} are as defined in paragraph (34) above, and L and R_{L}, and any group associated therewith, are each as defined in formula I above.

Suitably, in any of the definitions of formula I set out herein, R_{c} is as defined in any one of paragraphs (35) to (43) above. More suitably, R_{c} is as defined in any one of paragraphs (37) to (43) above. Even more suitably, R_{c} is as defined in any one of paragraphs (39) to (43) above. Yet more suitable, R_{c} is as defined in any one of paragraphs (40), (41), (42) or (43) above. Yet even more suitably, R_{c} is as defined in paragraphs (41), (42) or (43) above. Yet even more suitably, R_{c} is as defined in paragraphs (42) or (43) above.

In a particular group of compounds of formula I, R_{c} is as defined in paragraph (35) above, and L and R_{L}, and any group associated therewith, are each as defined in formula I above.

In a particular group of compounds of formula I, R_{c} is as defined in paragraph (37) above, and L and R_{L}, and any group associated therewith, are each as defined in formula I above.

In a particular group of compounds of formula I, R_{c} is as defined in paragraph (39) above, and L and R_{L}, and any group associated therewith, are each as defined in formula I above.

In a particular group of compounds of formula I, R_{c} is as defined in paragraph (40) above, and L and R_{L}, and any group associated therewith, are each as defined in formula I above.

In a particular group of compounds of formula I, R_{c} is as defined in paragraph (41) above, and L and R_{L}, and any group associated therewith, are each as defined in formula I above.

In a particular group of compounds of formula I, R_{c} is as defined in paragraph (42) above, and L and R_{L}, and any group associated therewith, are each as defined in formula I above.

In a particular group of compounds of formula I, R_{c} is as defined in paragraph (43) above, and L and R_{L}, and any group associated therewith, are each as defined in formula I above.

In a particular group of compounds of formula I defined herein:
L is as defined in any one of paragraphs (1) to (13) above;
R_{L} is as defined in any one of paragraphs (14) to (19) above;
Rₐ and Rₑ are both as defined in any one of paragraphs (20) to (23) above;
R_{b} and R_{d} are both as defined in any one of paragraphs (24) to (34) above; and
R_{c} is as defined in any one of paragraphs (34) to (43) above.

In a particular group of compounds of formula I defined herein:
L is as defined in any one of paragraphs (1) to (13) above;
R_{L} is as defined in any one of paragraphs (14) to (19) above;
Rₐ and Rₑ are both as defined in any one of paragraphs (21) to (23) above;
R_{b} and R_{d} are both as defined in any one of paragraphs (24) to (34) above; and
R_{c} is as defined in any one of paragraphs (34) to (43) above.

In a particular group of compounds of formula I defined herein:
L is as defined in any one of paragraphs (1) to (13) above;
R_{L} is as defined in any one of paragraphs (14) to (19) above;
Rₐ and Rₑ are both as defined in any one of paragraphs (22) or (23) above;
R_{b} and R_{d} are both as defined in any one of paragraphs (24) to (34) above; and
R_{c} is as defined in any one of paragraphs (34) to (43) above.

In a particular group of compounds of formula I defined herein:
L is as defined in any one of paragraphs (1) to (13) above;
R_{L} is as defined in any one of paragraphs (14) to (19) above;
Rₐ and Rₑ are both as defined in paragraph (23) above;
R_{b} and R_{d} are both as defined in any one of paragraphs (24) to (34) above; and
R_{c} is as defined in any one of paragraphs (34) to (43) above.

In a particular group of compounds of formula I defined herein:
L is as defined in any one of paragraphs (1) to (13) above;
R_{L} is as defined in any one of paragraphs (14) to (19) above;
Rₐ and Rₑ are both as defined in paragraph (23) above;
R_{b} and R_{d} are both as defined in any one of paragraphs (26) to (34) above; and
R_{c} is as defined in any one of paragraphs (34) to (43) above.

In a particular group of compounds of formula I defined herein:
L is as defined in any one of paragraphs (1) to (13) above;
R_{L} is as defined in any one of paragraphs (14) to (19) above;
Rₐ and Rₑ are both as defined in paragraph (23) above;
R_{b} and R_{d} are both as defined in any one of paragraphs (28) to (34) above; and
R_{c} is as defined in any one of paragraphs (34) to (43) above.

In a particular group of compounds of formula I defined herein:
L is as defined in any one of paragraphs (1) to (13) above;
R_{L} is as defined in any one of paragraphs (14) to (19) above;
Rₐ and Rₑ are both as defined in paragraph (23) above;
R_{b} and R_{d} are both as defined in any one of paragraphs (30) to (34) above; and
R_{c} is as defined in any one of paragraphs (34) to (43) above.

In a particular group of compounds of formula I defined herein:
L is as defined in any one of paragraphs (1) to (13) above;
R_{L} is as defined in any one of paragraphs (14) to (19) above;
Rₐ and Rₑ are both as defined in paragraph (23) above;
R_{b} and R_{d} are both as defined in any one of paragraphs (31) to (34) above; and
R_{c} is as defined in any one of paragraphs (34) to (43) above.

In a particular group of compounds of formula I defined herein:
L is as defined in any one of paragraphs (1) to (13) above;
R_{L} is as defined in any one of paragraphs (14) to (19) above;
Rₐ and Rₑ are both as defined in paragraph (23) above;
R_{b} and R_{d} are both as defined in any one of paragraphs (32) to (34) above; and
R_{c} is as defined in any one of paragraphs (34) to (43) above.

In a particular group of compounds of formula I defined herein:
L is as defined in any one of paragraphs (1) to (13) above;
R_{L} is as defined in any one of paragraphs (14) to (19) above;
Rₐ and Rₑ are both as defined in paragraph (23) above;
R_{b} and R_{d} are both as defined in any one of paragraphs (33) or (34) above; and
R_{c} is as defined in any one of paragraphs (34) to (43) above.

In a particular group of compounds of formula I defined herein:
L is as defined in any one of paragraphs (1) to (13) above;
R_{L} is as defined in any one of paragraphs (14) to (19) above;
Rₐ and Rₑ are both as defined in paragraph (23) above;
R_{b} and R_{d} are both as defined in paragraph (34) above; and
R_{c} is as defined in any one of paragraphs (34) to (43) above.

In a particular group of compounds of formula I defined herein:
L is as defined in any one of paragraphs (1) to (13) above;
R_{L} is as defined in any one of paragraphs (14) to (19) above;
Rₐ and Rₑ are both as defined in paragraph (23) above;
R_{b} and R_{d} are both as defined in any one of paragraphs (24) to (34) above; and
R_{c} is as defined in any one of paragraphs (36) to (43) above.

In a particular group of compounds of formula I defined herein:
L is as defined in any one of paragraphs (1) to (13) above;
R_{L} is as defined in any one of paragraphs (14) to (19) above;
Rₐ and Rₑ are both as defined in paragraph (23) above;
R_{b} and R_{d} are both as defined in any one of paragraphs (24) to (34) above; and
R_{c} is as defined in any one of paragraphs (38) to (43) above.

In a particular group of compounds of formula I defined herein:
L is as defined in any one of paragraphs (1) to (13) above;
R_{L} is as defined in any one of paragraphs (14) to (19) above;
Rₐ and Rₑ are both as defined in paragraph (23) above;
R_{b} and R_{d} are both as defined in any one of paragraphs (24) to (34) above; and
R_{c} is as defined in any one of paragraphs (40) to (43) above.

In a particular group of compounds of formula I defined herein:

L is as defined in any one of paragraphs (1) to (13) above;
R_{L} is as defined in any one of paragraphs (14) to (19) above;
Rₐ and Rₑ are both as defined in paragraph (23) above;
R_{b} and R_{d} are both as defined in any one of paragraphs (24) to (34) above; and
R_{c} is as defined in any one of paragraphs (41) to (43) above.

In a particular group of compounds of formula I defined herein:
L is as defined in any one of paragraphs (1) to (13) above;
R_{L} is as defined in any one of paragraphs (14) to (19) above;
Rₐ and Rₑ are both as defined in paragraph (23) above;
R_{b} and R_{d} are both as defined in any one of paragraphs (24) to (34) above; and
R_{c} is as defined in any one of paragraphs (42) or (43) above.

In a particular group of compounds of formula I defined herein:
L is as defined in any one of paragraphs (1) to (13) above;
R_{L} is as defined in any one of paragraphs (14) to (19) above;
Rₐ and Rₑ are both as defined in paragraph (23) above;
R_{b} and R_{d} are both as defined in any one of paragraphs (24) to (34) above; and
R_{c} is as defined in paragraph (43) above.

In a particular group of compounds of formula I defined herein:
L is as defined in any one of paragraphs (1) to (13) above;
R_{L} is as defined in any one of paragraphs (14) to (19) above;
Rₐ and Rₑ are both as defined in paragraph (23) above;
R_{b} and R_{d} are both as defined in any one of paragraphs (26) to (34) above; and
R_{c} is as defined in any one of paragraphs (36) to (43) above.

In a particular group of compounds of formula I defined herein:
L is as defined in any one of paragraphs (1) to (13) above;
R_{L} is as defined in any one of paragraphs (14) to (19) above;
Rₐ and Rₑ are both as defined in paragraph (23) above;
R_{b} and R_{d} are both as defined in any one of paragraphs (28) to (34) above; and
R_{c} is as defined in any one of paragraphs (38) to (43) above.

In a particular group of compounds of formula I defined herein:
L is as defined in any one of paragraphs (1) to (13) above;
R_{L} is as defined in any one of paragraphs (14) to (19) above;
Rₐ and Rₑ are both as defined in paragraph (23) above;
R_{b} and R_{d} are both as defined in any one of paragraphs (30) to (34) above; and
R_{c} is as defined in any one of paragraphs (40) to (43) above.

In a particular group of compounds of formula I defined herein:
L is as defined in any one of paragraphs (1) to (13) above;
R_{L} is as defined in any one of paragraphs (14) to (19) above;
Rₐ and Rₑ are both as defined in paragraph (23) above;
R_{b} and R_{d} are both as defined in any one of paragraphs (31) to (34) above; and
R_{c} is as defined in any one of paragraphs (40) to (43) above.

In a particular group of compounds of formula I defined herein:
L is as defined in any one of paragraphs (1) to (13) above;
R_{L} is as defined in any one of paragraphs (14) to (19) above;
Rₐ and Rₑ are both as defined in paragraph (23) above;
R_{b} and R_{d} are both as defined in any one of paragraphs (32) to (34) above; and
R_{c} is as defined in any one of paragraphs (41) to (43) above.

In a particular group of compounds of formula I defined herein:
L is as defined in any one of paragraphs (1) to (13) above;
R_{L} is as defined in any one of paragraphs (14) to (19) above;
Rₐ and Rₑ are both as defined in paragraph (23) above;
R_{b} and R_{d} are both as defined in any one of paragraphs (33) or (34) above; and
R_{c} is as defined in any one of paragraphs (42) or (43) above.

In a particular group of compounds of formula I defined herein:
L is as defined in any one of paragraphs (1) to (13) above;
R_{L} is as defined in any one of paragraphs (14) to (19) above;
Rₐ and Rₑ are both as defined in paragraph (23) above;
R_{b} and R_{d} are both as defined in paragraph (34) above; and
R_{c} is as defined in paragraph (43) above.

In a particular group of compounds of formula I defined herein:
L is as defined in any one of paragraphs (4) to (13) above;
R_{L} is as defined in any one of paragraphs (14) to (19) above;
Rₐ and Rₑ are both as defined in paragraph (23) above;
R_{b} and R_{d} are both as defined in any one of paragraphs (24) to (34) above; and
R_{c} is as defined in any one of paragraphs (34) to (43) above.

In a particular group of compounds of formula I defined herein:
L is as defined in any one of paragraphs (6) to (13) above;
R_{L} is as defined in any one of paragraphs (14) to (19) above;
Rₐ and Rₑ are both as defined in paragraph (23) above;
R_{b} and R_{d} are both as defined in any one of paragraphs (24) to (34) above; and
R_{c} is as defined in any one of paragraphs (34) to (43) above.

In a particular group of compounds of formula I defined herein:
L is as defined in any one of paragraphs (8) to (13) above;
R_{L} is as defined in any one of paragraphs (14) to (19) above;
Rₐ and Rₑ are both as defined in paragraph (23) above;
R_{b} and R_{d} are both as defined in any one of paragraphs (24) to (34) above; and
R_{c} is as defined in any one of paragraphs (34) to (43) above.

In a particular group of compounds of formula I defined herein:
L is as defined in any one of paragraphs (10) to (13) above;
R_{L} is as defined in any one of paragraphs (14) to (19) above;
Rₐ and Rₑ are both as defined in paragraph (23) above;
R_{b} and R_{d} are both as defined in any one of paragraphs (24) to (34) above; and
R_{c} is as defined in any one of paragraphs (34) to (43) above.

In a particular group of compounds of formula I defined herein:
L is as defined in any one of paragraphs (11) to (13) above;
R_{L} is as defined in any one of paragraphs (14) to (19) above;
Rₐ and Rₑ are both as defined in paragraph (23) above;
R_{b} and R_{d} are both as defined in any one of paragraphs (24) to (34) above; and
R_{c} is as defined in any one of paragraphs (34) to (43) above.

In a particular group of compounds of formula I defined herein:
L is as defined in any one of paragraphs (12) or (13) above;
R_{L} is as defined in any one of paragraphs (14) to (19) above;
Rₐ and Rₑ are both as defined in paragraph (23) above;
R_{b} and R_{d} are both as defined in any one of paragraphs (24) to (34) above; and
R_{c} is as defined in any one of paragraphs (34) to (43) above.

In a particular group of compounds of formula I defined herein:
L is as defined in paragraph (13) above;
R_{L} is as defined in any one of paragraphs (14) to (19) above;
Rₐ and Rₑ are both as defined in paragraph (23) above;
R_{b} and R_{d} are both as defined in any one of paragraphs (24) to (34) above; and
R_{c} is as defined in any one of paragraphs (34) to (43) above.

In a particular group of compounds of formula I defined herein:
L is as defined in any one of paragraphs (1) to (13) above;
R_{L} is as defined in any one of paragraphs (15) to (19) above;
Rₐ and Rₑ are both as defined in paragraph (23) above;
R_{b} and R_{d} are both as defined in any one of paragraphs (24) to (34) above; and
R_{c} is as defined in any one of paragraphs (34) to (43) above.

In a particular group of compounds of formula I defined herein:
L is as defined in any one of paragraphs (1) to (13) above;
R_{L} is as defined in any one of paragraphs (16) to (19) above;
Rₐ and Rₑ are both as defined in paragraph (23) above;
R_{b} and R_{d} are both as defined in any one of paragraphs (24) to (34) above; and
R_{c} is as defined in any one of paragraphs (34) to (43) above.

In a particular group of compounds of formula I defined herein:
L is as defined in any one of paragraphs (1) to (13) above;
R_{L} is as defined in any one of paragraphs (17) to (19) above;
Rₐ and Rₑ are both as defined in paragraph (23) above;
R_{b} and R_{d} are both as defined in any one of paragraphs (24) to (34) above; and
R_{c} is as defined in any one of paragraphs (34) to (43) above.

In a particular group of compounds of formula I defined herein:
L is as defined in any one of paragraphs (1) to (13) above;
R_{L} is as defined in any one of paragraphs (18) or (19) above;
Rₐ and Rₑ are both as defined in paragraph (23) above;
R_{b} and R_{d} are both as defined in any one of paragraphs (24) to (34) above; and
R_{c} is as defined in any one of paragraphs (34) to (43) above.

In a particular group of compounds of formula I defined herein:
L is as defined in any one of paragraphs (1) to (13) above;
R_{L} is as defined in paragraph (19) above;
Rₐ and Rₑ are both as defined in paragraph (23) above;
R_{b} and R_{d} are both as defined in any one of paragraphs (24) to (34) above; and
R_{c} is as defined in any one of paragraphs (34) to (43) above.

In a particular group of compounds of formula I defined herein:
L is as defined in any one of paragraphs (4) to (13) above;
R_{L} is as defined in any one of paragraphs (15) to (19) above;
Rₐ and Rₑ are both as defined in paragraph (23) above;
R_{b} and R_{d} are both as defined in any one of paragraphs (26) to (34) above; and
R_{c} is as defined in any one of paragraphs (36) to (43) above.

In a particular group of compounds of formula I defined herein:
L is as defined in any one of paragraphs (6) to (13) above;
R_{L} is as defined in any one of paragraphs (16) to (19) above;
Rₐ and Rₑ are both as defined in paragraph (23) above;
R_{b} and R_{d} are both as defined in any one of paragraphs (28) to (34) above; and
R_{c} is as defined in any one of paragraphs (38) to (43) above.

In a particular group of compounds of formula I defined herein:
L is as defined in any one of paragraphs (8) to (13) above;
R_{L} is as defined in any one of paragraphs (17) to (19) above;
Rₐ and Rₑ are both as defined in paragraph (23) above;
R_{b} and R_{d} are both as defined in any one of paragraphs (30) to (34) above; and
R_{c} is as defined in any one of paragraphs (40) to (43) above.

In a particular group of compounds of formula I defined herein:
L is as defined in any one of paragraphs (10) to (13) above;
R_{L} is as defined in any one of paragraphs (18) or (19) above;
Rₐ and Rₑ are both as defined in paragraph (23) above;
R_{b} and R_{d} are both as defined in any one of paragraphs (31) to (34) above; and
R_{c} is as defined in any one of paragraphs (40) to (43) above.

In a particular group of compounds of formula I defined herein:
L is as defined in any one of paragraphs (11) to (13) above;
R_{L} is as defined in any one of paragraphs (18) or (19) above;
Rₐ and Rₑ are both as defined in paragraph (23) above;
R_{b} and R_{d} are both as defined in any one of paragraphs (32) to (34) above; and
R_{c} is as defined in any one of paragraphs (41) to (43) above.

In a particular group of compounds of formula I defined herein:
L is as defined in any one of paragraphs (12) or (13) above;
R_{L} is as defined in any one of paragraphs (18) or (19) above;
Rₐ and Rₑ are both as defined in paragraph (23) above;
R_{b} and R_{d} are both as defined in any one of paragraphs (33) or (34) above; and
R_{c} is as defined in any one of paragraphs (42) or (43) above.

In a particular group of compounds of formula I defined herein:
L is as defined in paragraph (13) above;
R_{L} is as defined in paragraph (19) above;
Rₐ and Rₑ are both as defined in paragraph (23) above;
R_{b} and R_{d} are both as defined in paragraph (34) above; and
R_{c} is as defined in paragraph (43) above.

Suitably, in any of the definitions of formula I set out herein, R₁₀₀ is as defined in formula I above, or as defined in any one of paragraphs (44) or (45) above.

Suitably, in any of the definitions of formula I set out herein, R₁₀₀ₐ is as defined in formula I above, or as defined in any one of paragraphs (46) or (47) above.

Suitably, in any of the definitions of formula I set out herein, R₁₀₀ and R₁₀₀ₐ are as defined in formula I above, or as defined in any one of paragraphs (48) or (49) above.

Suitably, in any of the definitions of formula I set out herein, R₁₀₁ is as defined in formula I above, or as defined in any one of paragraphs (50), (51) or (52) above.

Suitably, in any of the definitions of formula I set out herein, R₁₀₁ₐ is as defined in formula I above, or as defined in any one of paragraphs (53) or (54) above.

Suitably, in any of the definitions of formula I set out herein, R₁₀₁ and R₁₀₁ₐ are as defined in formula I above, or as defined in any one of paragraphs (55) or (56) above.

Suitably, in any of the definitions of formula I set out herein, integer a is as defined in formula I above, or as defined in any one of paragraphs (57), (58) or (59) above.

Suitably, in any of the definitions of formula I set out herein, Q₁ is as defined in formula I above, or as defined in any one of paragraphs (60), (61) or (62) above.

Suitably, in any of the definitions of formula I set out herein, R₁₀₂ is as defined in formula I above, or as defined in any one of paragraphs (63) or (64) above.

Suitably, in any of the definitions of formula I set out herein, Q₂, Q₃, Q₄, Q₅ and Q₆ is as defined in formula I above, or as defined in any one of paragraphs (66) to (71) above. More, suitably, Q₂, Q₃, Q₄, Q₅ and Q₆ is as defined in formula I above, or as defined in any one of paragraphs (67) to (71) above. Even more suitably, Q₂, Q₃, Q₄, Q₅ and Q₆ is as defined in formula I above, or as defined in any one of paragraphs (68) to (71) above. Yet more suitably, Q₂, Q₃, Q₄, Q₅ and Q₆ is as defined in formula I above, or as defined in any one of paragraphs (69) to (71) above. Most suitably, Q₂, Q₃, Q₄, Q₅ and Q₆ is as defined in formula I above, or as defined in any one of paragraphs (70) or (71) above.

Suitably, in any of the definitions of formula I set out herein, Q₇, Q₈, Q₉ and Q₁₀ is as defined in formula I above, or as defined in any one of paragraphs (72) to (74) above. More suitably, in any of the definitions of formula I set out herein, Q₇, Q₈, Q₉ and Q₁₀ is as defined in formula I above, or as defined in any one of paragraphs (73) or (74) above.

Suitably, in any of the definitions of formula I set out herein, ring A is as defined in formula I above, or as defined in any one of paragraphs (75) to (80) above. More suitably, ring A is as defined in any one of paragraphs (76) to (80) above. Even more suitably, ring A is as defined in any one of paragraphs (77) to (80) above. Yet more suitably, ring A is as defined in any one of paragraphs (78), (79) or (80) above. Most suitable, ring A is as defined in anyone of paragraph (79) or (80) above.

Suitably, in any of the definitions of formula I set out herein, R_{R} is as defined in formula I above or as defined in any one of paragraphs (81) or (82) above.

In a particular group of compounds of formula I, R_{R} is as defined in paragraph (81) above, and L and R_{L}, and any group associated therewith, are each as defined in formula I above.

In a particular group of compounds of formula I, R_{R} is as defined in paragraph (82) above, and L and R_{L}, and any group associated therewith, are each as defined in formula I above.

Suitably, in any of the definitions of formula I set out herein, A₁ and A₂ are as defined in paragraph (83) or (84) above.

Suitably, in any of the definitions of formula I set out herein, R₁ is as defined in any one of paragraphs (85) to (91) above. More suitably, R₁ is as defined in any one of paragraphs (86) to (91) above. Even more suitably, R₁ is as defined in any one of paragraphs (87) to (91) above. Yet more suitably, R₁ is as defined in any one of paragraphs (88) to (91) above. Yet even more suitably, R₁ is as defined in any one of paragraphs (89) to (91) above. Most suitably, R₁ is as defined in any one of paragraphs (90) or (91) above.

Suitably, in any of the definitions of formula I set out herein, X₁ is as defined in any one of paragraphs (92) to (96) above. More suitably, X₁ is as defined in any one of paragraphs (94), (95) or (96) above.

Suitably, in any of the definitions of formula I set out herein, X₅ is as defined in paragraph (97) above.

Suitably, in any of the definitions of formula I set out herein, X₂, X₃ and X₄ are as defined in any one of paragraphs (98) to (105) above. More suitably, X₂, X₃ and X₄ are as defined in any one of paragraphs (100) to (105) above. Even more suitably, X₂, X₃ and X₄ are as defined in any one of paragraphs (102) to (105) above.

Suitably, in any of the definitions of formula I set out herein, X₆, X₇, X₈ and X₉ are as defined in any one of paragraphs (106) to (112) above. More suitably, X₆, X₇, Xa, and X₉ are as defined in any one of paragraphs (108) to (112) above. Even more suitably, X₆, X₇, X₈ and Xe are as defined in any one of paragraphs (110) to (112) above. Yet even more suitably, X₆, X₇, X₈ and X₉ are as defined in any one of paragraphs (111) or (112) above.

Suitably, in any of the definitions of formula I set out herein, X₁₀, X₁₁ and X₁₂ are as defined in any one of paragraphs (113) to (117) above. More suitably, X₁₀, X₁₁ and X₁₂ are as defined in any one of paragraphs (115) to (117) above. Even more suitably, X₁₀, X₁₁ and X₁₂ are as defined in any one of paragraphs (116) or (117) above.

Suitably, in any of the definitions of formula I set out herein, X₁₃, X₁₄, X₁₅, X₁₆ and X₁₇ are as defined in any one of paragraphs (118) to (126) above. More suitably, X₁₃, X₁₄, X₁₅, X₁₆ and X₁₇ are as defined in any one of paragraphs (120) to (126) above. Even more suitably, X₁₃, X₁₄, X₁₅, X₁₆ and X₁₇ are as defined in any one of paragraphs (122) to (126) above. Yet even more suitably, X₁₃, X₁₄, X₁₅, X₁₆ and X₁₇ are as defined in any one of paragraphs (124) to (126) above.

Suitably, in any of the definitions of formula I set out herein, R_{1A} is as defined in any one of paragraphs (127) to (132). More suitably, R_{1A} is as defined in any one of paragraphs (129) to (132). Even more suitably, R_{1A} is as defined in any one of paragraphs (130) to (132).

Even more suitably, R_{1A} is as defined in any one of paragraphs (131) or (132). Most suitably, R_{1A} is as defined in paragraph (132).

Suitably, in any of the definitions of formula I set out herein, R_{1B} is as defined in any one of paragraphs (133) or (134) above.

Suitably, in any of the definitions of formula I set out herein, A₃ is as defined in any one of paragraphs (135), (136) or (137) above.

Suitably, in any of the definitions of formula I set out herein, A₄ is as defined in any one of paragraphs (138), (139) or (140) above.

Suitably, in any of the definitions of formula I set out herein, A₅ and A₆ are as defined in any one of paragraphs (141) or (142) above.

Suitably, in any of the definitions of formula I set out herein, A₇ is as defined in any one of paragraphs (143), (144) or (145) above.

Suitably, in any of the definitions of formula I set out herein, R₂ is as defined in any one of paragraphs (146) to (153) above. More suitably, R₂ is as defined in any one of paragraphs (147) to (153) above. Even more suitably, R₂ is as defined in any one of paragraphs (148) to (153) above. Yet more suitably, R₂ is as defined in any one of paragraphs (149) to (153) above. Yet even more suitably, R₂ is as defined in any one of paragraphs (150) to (153) above. Yet still even more suitably, R₂ is as defined in any one of paragraphs (151), (152) or (153) above. Most suitably, R₂ is as defined in any one of paragraphs (152) or (153) above.

Suitably, in any of the definitions of formula I set out herein, R₃ is as defined in any one of paragraphs (154) or (155) above.

Suitably, in any of the definitions of formula I set out herein, R₄ is as defined in any one of paragraphs (156) or (157) above.

Suitably, in any of the definitions of formula I set out herein, R₇ is as defined in any one of paragraphs (158) or (159) above.

Suitably, in any of the definitions of formula I set out herein, Aₛ is as defined in paragraph (160) above.

Suitably, in any of the definitions of formula I set out herein, A₉ is as defined in paragraph (161) above.

Suitably, in any of the definitions of formula I set out herein, A₁₀ is as defined in paragraph (162) above.

Suitably, in any of the definitions of formula I set out herein, R₈, R₉ and R₁₀ are as defined in paragraph (163) above.

In a particular group of compounds of formula I defined herein:
R_{R} is as defined in any one of paragraphs (81) or (82) above;
R₁ is as defined in any one of paragraphs (85) to (91) above, or R₂ is as defined in any one of paragraphs (146) to (153) above;
L is as defined in any one of paragraphs (1) to (13) above; and
R_{L}, and any groups associated therewith, is as defined herein.

In a particular group of compounds of formula I defined herein:
R_{R} is as defined in any one of paragraphs (81) or (82) above;
R₁ is as defined in any one of paragraphs (86) to (91) above, or R₂ is as defined in any one of paragraphs (148) to (153) above;
L is as defined in any one of paragraphs (1) to (13) above; and
R_{L}, and any groups associated therewith, is as defined herein.

In a particular group of compounds of formula I defined herein:
R_{R} is as defined in any one of paragraphs (81) or (82) above;
R₁ is as defined in any one of paragraphs (87) to (91) above, or R₂ is as defined in any one of paragraphs (149) to (153) above;
L is as defined in any one of paragraphs (1) to (13) above; and
R_{L}, and any groups associated therewith, is as defined herein.

In a particular group of compounds of formula I defined herein:
R_{R} is as defined in any one of paragraphs (81) or (82) above;
R₁ is as defined in any one of paragraphs (88) to (91) above, or R₂ is as defined in any one of paragraphs (150) to (153) above;
L is as defined in any one of paragraphs (1) to (13) above; and
R_{L}, and any groups associated therewith, is as defined herein.

In a particular group of compounds of formula I defined herein:
R_{R} is as defined in any one of paragraphs (81) or (82) above;
R₁ is as defined in any one of paragraphs (89) to (91) above, or R₂ is as defined in any one of paragraphs (151) to (153) above;
L is as defined in any one of paragraphs (1) to (13) above; and
R_{L}, and any groups associated therewith, is as defined herein.

In a particular group of compounds of formula I defined herein:
R_{R} is as defined in any one of paragraphs (81) or (82) above;
R₁ is as defined in any one of paragraphs (90) or (91) above, or R₂ is as defined in any one of paragraphs (152) or (153) above;
L is as defined in any one of paragraphs (1) to (13) above; and
R_{L}, and any groups associated therewith, is as defined herein.

In a particular group of compounds of formula I defined herein:
R_{R} is as defined in any one of paragraphs (81) or (82) above;
R₁ is as defined in paragraph (91) above, or R₂ is as defined in paragraph (153) above;
L is as defined in any one of paragraphs (1) to (13) above; and
R_{L}, and any groups associated therewith, is as defined herein.

In a particular group of compounds of formula I defined herein:
R_{R} is as defined in any one of paragraphs (81) or (82) above;
R₁ is as defined in any one of paragraphs (86) to (91) above, or R₂ is as defined in any one of paragraphs (148) to (153) above;
L is as defined in any one of paragraphs (4) to (13) above;
R_{L} is as defined in any one of paragraphs (14) to (19) above;
Rₐ and Rₑ are both as defined in any one of paragraphs (20) to (23) above;
R_{b} and R_{d} are both as defined in any one of paragraphs (24) to (34) above; and
R_{c} is as defined in any one of paragraphs (34) to (43) above.

In a particular group of compounds of formula I defined herein:
R_{R} is as defined in any one of paragraphs (81) or (82) above;
R₁ is as defined in any one of paragraphs (87) to (91) above, or R₂ is as defined in any one of paragraphs (149) to (153) above;
L is as defined in any one of paragraphs (8) to (13) above;
R_{L} is as defined in any one of paragraphs (15) to (19) above;
Rₐ and Rₑ are both as defined in any one of paragraphs (21) to (23) above;
R_{b} and R_{d} are both as defined in any one of paragraphs (26) to (34) above; and
R_{c} is as defined in any one of paragraphs (36) to (43) above.

In a particular group of compounds of formula I defined herein:
R_{R} is as defined in any one of paragraphs (81) or (82) above;
R₁ is as defined in any one of paragraphs (88) to (91) above, or R₂ is as defined in any one of paragraphs (150) to (153) above;
L is as defined in any one of paragraphs (9) to (13) above;
R_{L} is as defined in any one of paragraphs (16) to (19) above;
Rₐ and Rₑ are both as defined in any one of paragraphs (22) or (23) above;
R_{b} and R_{d} are both as defined in any one of paragraphs (28) to (34) above; and
R_{c} is as defined in any one of paragraphs (38) to (43) above.

In a particular group of compounds of formula I defined herein:
R_{R} is as defined in any one of paragraphs (81) or (82) above;
R₁ is as defined in any one of paragraphs (89) to (91) above, or R₂ is as defined in any one of paragraphs (151) to (153) above;
L is as defined in any one of paragraphs (10) to (13) above;
R_{L} is as defined in any one of paragraphs (17) to (19) above;
Rₐ and Rₑ are both as defined in paragraph (23) above;
R_{b} and R_{d} are both as defined in any one of paragraphs (30) to (34) above; and
R_{c} is as defined in any one of paragraphs (40) to (43) above.

In a particular group of compounds of formula I defined herein:
R_{R} is as defined in any one of paragraphs (81) or (82) above;
R₁ is as defined in any one of paragraphs (90) or (91) above, or R₂ is as defined in any one of paragraphs (152) or (153) above;
L is as defined in any one of paragraphs (11) to (13) above;
R_{L} is as defined in any one of paragraphs (18) or (19) above;
Rₐ and Rₑ are both as defined in paragraph (23) above;
R_{b} and R_{d} are both as defined in any one of paragraphs (32) to (34) above; and
R_{c} is as defined in any one of paragraphs (42) or (43) above.

In a particular group of compounds of formula I defined herein:
R_{R} is as defined in any one of paragraphs (81) or (82) above;
R₁ is as defined in paragraph (91) above, or R₂ is as defined in paragraph (153) above;
L is as defined in any one of paragraphs (12) or (13) above;
R_{L} is as defined in paragraph (19) above;
R_{b} and R_{d} are both as defined in paragraph (34) above; and
R_{c} is as defined in paragraph (43) above.

In a particular group of compounds of the invention, the compound is a compound of formula I defined herein in which R_{L} is as defined in paragraph (16) above Rₐ and Rₑ are as defined in paragraph (23) above, i.e. the compounds have the formula Ip shown below, or a pharmaceutically acceptable salt thereof:

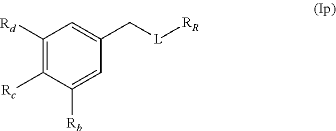

wherein L, R_{R}, R_{b}, R_{c} and R_{d}, and any groups associated therewith, each have any one of the definitions set out hereinbefore.

In a particular group of compounds of formula Ip:
L is as defined in any one of paragraphs (1) to (13) above;
R_{R} is as defined in any one of paragraphs (81) or (82) above;
R₁ is as defined in any one of paragraphs (86) to (91) above, or R₂ is as defined in any one of paragraphs (148) to (153) above;
R_{b} and R_{d} are both as defined in any one of paragraphs (24) to (34) above; and
R_{c} is as defined in any one of paragraphs (34) to (43) above.

In a particular group of compounds of formula Ip:
L is as defined in any one of paragraphs (4) to (13) above;
R_{R} is as defined in any one of paragraphs (81) or (82) above;
R₁ is as defined in any one of paragraphs (87) to (91) above, or R₂ is as defined in any one of paragraphs (149) to (153) above;
R_{b} and R_{d} are both as defined in any one of paragraphs (26) to (34) above; and
R_{c} is as defined in any one of paragraphs (36) to (43) above.

In a particular group of compounds of formula Ip:
L is as defined in any one of paragraphs (6) to (13) above;
R_{R} is as defined in any one of paragraphs (81) or (82) above;
R₁ is as defined in any one of paragraphs (88) to (91) above, or R₂ is as defined in any one of paragraphs (150) to (153) above;
R_{b} and R_{d} are both as defined in any one of paragraphs (28) to (34) above; and
R_{c} is as defined in any one of paragraphs (38) to (43) above.

In a particular group of compounds of formula Ip:
L is as defined in any one of paragraphs (8) to (13) above;
R_{R} is as defined in any one of paragraphs (81) or (82) above;
R₁ is as defined in any one of paragraphs (89) to (91) above, or R₂ is as defined in any one of paragraphs (151) to (153) above;
R_{b} and R_{d} are both as defined in any one of paragraphs (30) to (34) above; and
R_{c} is as defined in any one of paragraphs (40) to (43) above.

In a particular group of compounds of formula Ip:
L is as defined in any one of paragraphs (10) to (13) above;
R_{R} is as defined in any one of paragraphs (81) or (82) above;
R₁ is as defined in any one of paragraphs (90) or (91) above, or R₂ is as defined in any one of paragraphs (152) or (153) above;
R_{b} and R_{d} are both as defined in any one of paragraphs (31) to (34) above; and
R_{c} is as defined in any one of paragraphs (40) to (43) above.

In a particular group of compounds of formula Ip:
L is as defined in any one of paragraphs (11) to (13) above;
R_{R} is as defined in any one of paragraphs (81) or (82) above;
R₁ is as defined in any one of paragraphs (90) or (91) above, or R₂ is as defined in any one of paragraphs (152) or (153) above;
R_{b} and R_{d} are both as defined in any one of paragraphs (32) to (34) above; and
R_{c} is as defined in any one of paragraphs (41) to (43) above.

In a particular group of compounds of formula Ip:
L is as defined in any one of paragraphs (12) or (13) above;
R_{R} is as defined in any one of paragraphs (81) or (82) above;
R₁ is as defined in paragraph (91) above, or R₂ is as defined in paragraph (153) above;
Rₐ and Rₑ are both as defined in paragraph (23) above;
R_{b} and R_{d} are both as defined in any one of paragraphs (33) or (34) above; and
R_{c} is as defined in any one of paragraphs (42) or (43) above.

In a particular group of compounds of formula Ip defined herein:
L is as defined in paragraph (13) above;
R_{R} is as defined in paragraph (82) above;
R₁ is as defined in paragraph (91) above;
R_{b} and R_{d} are both as defined in paragraph (34) above; and
R_{c} is as defined in paragraph (43) above.

In a particular group of compounds of the invention, the compound is a compound of formula I defined herein in which R_{L} is as defined in paragraph (16) above and Rₑ is as defined in paragraph (23) above, i.e. the compounds have the formula Iq shown below, or a pharmaceutically acceptable salt thereof:

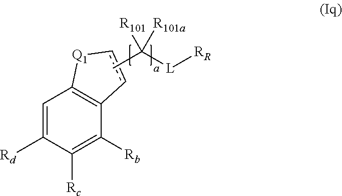

wherein L, R_{R}, a, R₁₀₁, R₁₀₁ₐ, Q₁, R_{b}, R_{c} and R_{d} each have any one of the definitions set out herein.

In a particular group of compounds of formula Iq:
L is as defined in any one of paragraphs (1) to (13) above;
R_{R} is as defined in any one of paragraphs (81) or (82) above;
R₁ is as defined in any one of paragraphs (86) to (91) above, or R₂ is as defined in any one of paragraphs (148) to (153) above;
Integer a is as defined in paragraph (57) to (59) above;
R₁₀₁ is as defined in any one of paragraphs (50) to (52) above;
R₁₀₁ₐ is as defined in any one of paragraphs (53) or (54) above;
Q₁ is as defined in any one of paragraphs (60) to (62) above;
R_{b} and R_{d} are both as defined in any one of paragraphs (24) to (34) above; and
R_{c} is as defined in any one of paragraphs (34) to (43) above.

In a particular group of compounds of formula Iq:
L is as defined in any one of paragraphs (4) to (13) above;
R_{R} is as defined in any one of paragraphs (81) or (82) above;
R₁ is as defined in any one of paragraphs (87) to (91) above, or R₂ is as defined in any one of paragraphs (149) to (153) above;
Integer a is as defined in paragraph (58) or (59) above;
R₁₀₁ is as defined in any one of paragraphs (51) or (52) above;
R₁₀₁ₐ is as defined in any one of paragraphs (53) or (54) above;
Q₁ is as defined in any one of paragraphs (61) to (62) above;
R_{b} and R_{d} are both as defined in any one of paragraphs (26) to (34) above; and
R_{c} is as defined in any one of paragraphs (36) to (43) above.

In a particular group of compounds of formula Iq:
L is as defined in any one of paragraphs (6) to (13) above;
R_{R} is as defined in any one of paragraphs (81) or (82) above;
R₁ is as defined in any one of paragraphs (88) to (91) above, or R₂ is as defined in any one of paragraphs (150) to (153) above;
Integer a is as defined in paragraph (58) or (59) above;
R₁₀₁ is as defined in any one of paragraphs (51) or (52) above;
R₁₀₁ₐ is as defined in any one of paragraphs (53) or (54) above;
Q₁ is as defined in any one of paragraphs (61) to (62) above;
R_{b} and R_{d} are both as defined in any one of paragraphs (28) to (34) above; and
R_{c} is as defined in any one of paragraphs (38) to (43) above.

In a particular group of compounds of formula Iq:
L is as defined in any one of paragraphs (8) to (13) above;
R_{R} is as defined in any one of paragraphs (81) or (82) above;
R₁ is as defined in any one of paragraphs (89) to (91) above, or R₂ is as defined in any one of paragraphs (151) to (153) above;
Integer a is as defined in paragraph (58) or (59) above;
R₁₀₁ is as defined in any one of paragraphs (51) or (52) above;
R₁₀₁ₐ is as defined in any one of paragraphs (53) or (54) above;
Q₁ is as defined in any one of paragraphs (61) to (62) above;
R_{b} and R_{d} are both as defined in any one of paragraphs (30) to (34) above; and
R_{c} is as defined in any one of paragraphs (40) to (43) above.

In a particular group of compounds of formula Iq:
L is as defined in any one of paragraphs (10) to (13) above;
R_{R} is as defined in any one of paragraphs (81) or (82) above;
R₁ is as defined in any one of paragraphs (90) or (91) above, or R₂ is as defined in any one of paragraphs (152) or (153) above;
Integer a is as defined in paragraph (58) or (59) above;
R₁₀₁ is as defined in any one of paragraphs (51) or (52) above;
R₁₀₁ₐ is as defined in any one of paragraphs (53) or (54) above;
Q₁ is as defined in any one of paragraphs (61) to (62) above;
R_{b} and R_{d} are both as defined in any one of paragraphs (31) to (34) above; and
R_{c} is as defined in any one of paragraphs (40) to (43) above.

In a particular group of compounds of formula Iq:
L is as defined in any one of paragraphs (11) to (13) above;
R_{R} is as defined in any one of paragraphs (81) or (82) above;
R₁ is as defined in any one of paragraphs (90) or (91) above, or R₂ is as defined in any one of paragraphs (152) or (153) above;
Integer a is as defined in paragraph (58) or (59) above;
R₁₀₁ is as defined in any one of paragraphs (51) or (52) above;
R₁₀₁ₐ is as defined in any one of paragraphs (53) or (54) above;
Q₁ is as defined in any one of paragraphs (61) to (62) above;
R₁₀₂ is as defined in any one of paragraphs (63) or (64) above;
R_{b} and R_{d} are both as defined in any one of paragraphs (32) to (34) above; and
R_{c} is as defined in any one of paragraphs (41) to (43) above.

In a particular group of compounds of formula Iq:
L is as defined in any one of paragraphs (12) or (13) above;
R_{R} is as defined in any one of paragraphs (81) or (82) above;
R₁ is as defined in paragraph (91) above, or R₂ is as defined in paragraph (153) above;
Integer a is as defined in paragraph (58) or (59) above;
R₁₀₁ is as defined in any one of paragraphs (51) or (52) above;
R₁₀₁ₐ is as defined in any one of paragraphs (53) or (54) above;
Q₁ is as defined in any one of paragraphs (61) to (62) above;
R₁₀₂ is as defined in any one of paragraphs (63) or (64) above;
Rₐ and Rₑ are both as defined in paragraph (23) above;
R_{b} and R_{d} are both as defined in any one of paragraphs (33) or (34) above; and
R_{c} is as defined in any one of paragraphs (42) or (43) above.

In a particular group of compounds of formula Iq defined herein:
L is as defined in paragraph (13) above;
R_{R} is as defined in paragraph (82) above;
R₁ is as defined in paragraph (91) above;
Integer a is as defined in paragraph (58) or (59) above;
R₁₀₁ is as defined in any one of paragraphs (51) or (52) above;
R₁₀₁ₐ is as defined in any one of paragraphs (53) or (54) above;
Q₁ is as defined in any one of paragraphs (61) to (62) above;
R₁₀₂ is as defined in paragraph (64) above;
R_{b} and R_{d} are both as defined in paragraph (34) above; and
R_{c} is as defined in paragraph (43) above.

In a particular group of compounds of the invention, the compound is a compound of formula I defined herein in which R_{R} is as defined in paragraph (81) above, i.e. the compounds have the formula Ir shown below, or a pharmaceutically acceptable salt thereof:

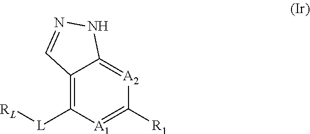

wherein A₁, A₂, L, R_{L} and R₁ each have any one of the definitions set out herein.

In a particular group of compounds of formula Ir:
A₁ and A₂ are as defined in any one of paragraphs (83) or (84) above;
L is as defined in any one of paragraphs (1) to (13) above;
R_{L} is as defined in any one of paragraphs (14) to (19) above;
R₁ is as defined in any one of paragraphs (85) to (91) above;
R_{1A} is as defined in any one of paragraphs (127) to (132) above; and
R_{1B} is as defined in any one of paragraphs (133) or (134) above.

In a particular group of compounds of formula Ir:
A₁ and A₂ are as defined in any one of paragraphs (83) or (84) above;
L is as defined in any one of paragraphs (4) to (13) above;
R_{L} is as defined in any one of paragraphs (15) to (19) above;
R₁ is as defined in any one of paragraphs (86) to (91) above;
R_{1A} is as defined in any one of paragraphs (128) to (132) above; and
R_{1B} is as defined in any one of paragraphs (133) or (134) above.

In a particular group of compounds of formula Ir:
A₁ and A₂ are as defined in any one of paragraphs (83) or (84) above;
L is as defined in any one of paragraphs (6) to (13) above;
R_{L} is as defined in any one of paragraphs (16) to (19) above;
R₁ is as defined in any one of paragraphs (87) to (91) above;
R_{1A} is as defined in any one of paragraphs (129) to (132) above; and
R_{1B} is as defined in any one of paragraphs (133) or (134) above.

In a particular group of compounds of formula Ir:
A₁ and A₂ are as defined in any one of paragraphs (83) or (84) above;
L is as defined in any one of paragraphs (8) to (13) above;
R_{L} is as defined in any one of paragraphs (17) to (19) above;
R₁ is as defined in any one of paragraphs (88) to (91) above;
R_{1A} is as defined in any one of paragraphs (130) to (132) above; and
R_{1B} is as defined in any one of paragraphs (133) or (134) above.

In a particular group of compounds of formula Ir:
A₁ and A₂ are as defined in any one of paragraphs (83) or (84) above;
L is as defined in any one of paragraphs (10) to (13) above;
R_{L} is as defined in any one of paragraphs (18) or (19) above;
R₁ is as defined in any one of paragraphs (89) to (91) above;
R_{1A} is as defined in any one of paragraphs (131) or (132) above; and
R_{1B} is as defined in any one of paragraphs (133) or (134) above.

In a particular group of compounds of formula Ir:
A₁ and A₂ are as defined in any one of paragraphs (83) or (84) above;
L is as defined in any one of paragraphs (12) or (13) above;
R_{L} is as defined in any one of paragraphs (18) or (19) above;
R₁ is as defined in any one of paragraphs (90) or (91) above;
R_{1A} is as defined in any one of paragraphs (131) or (132) above; and
R_{1B} is as defined in any one of paragraphs (133) or (134) above.

In a particular group of compounds of formula Ir:
A₁ and A₂ are as defined in paragraph (83) above;
L is as defined in paragraph (13) above;
R_{L} is as defined in paragraph (19) above;
R₁ is as defined in paragraph (91) above;
R_{1A} is as defined in paragraph (132) above; and
R_{1B} is as defined in paragraph (134) above.

In a particular group of compounds of the invention, the compound is a compound of formula I defined herein in which R_{R} is as defined in paragraph (81) above and A₁ and A₂ are as defined in paragraph (83) above, i.e. the compounds have the formula Is shown below, or a pharmaceutically acceptable salt thereof:

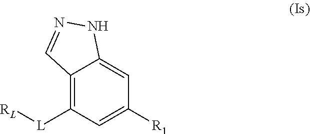

wherein L, R_{L} and R₁ each have any one of the definitions set out herein.

In a particular group of compounds of formula Is:
L is as defined in any one of paragraphs (1) to (13) above;
R_{L} is as defined in any one of paragraphs (14) to (19) above;
R₁ is as defined in any one of paragraphs (85) to (91) above;
R_{1A} is as defined in any one of paragraphs (127) to (132) above; and
R_{1B} is as defined in any one of paragraphs (133) or (134) above.

In a particular group of compounds of formula Is:
L is as defined in any one of paragraphs (4) to (13) above;
R_{L} is as defined in any one of paragraphs (15) to (19) above;
R₁ is as defined in any one of paragraphs (86) to (91) above;
R_{1A} is as defined in any one of paragraphs (128) to (132) above; and
R_{1B} is as defined in any one of paragraphs (133) or (134) above.

In a particular group of compounds of formula Is:
L is as defined in any one of paragraphs (6) to (13) above;
R_{L} is as defined in any one of paragraphs (16) to (19) above;
R₁ is as defined in any one of paragraphs (87) to (91) above;
R_{1A} is as defined in any one of paragraphs (129) to (132) above; and
R_{1B} is as defined in any one of paragraphs (133) or (134) above.

In a particular group of compounds of formula Is:
L is as defined in any one of paragraphs (8) to (13) above;
R_{L} is as defined in any one of paragraphs (17) to (19) above;
R₁ is as defined in any one of paragraphs (88) to (91) above;
R_{1A} is as defined in any one of paragraphs (130) to (132) above; and
R_{1B} is as defined in any one of paragraphs (133) or (134) above.

In a particular group of compounds of formula Is:
L is as defined in any one of paragraphs (10) to (13) above;
R_{L} is as defined in any one of paragraphs (18) or (19) above;
R₁ is as defined in any one of paragraphs (89) to (91) above;
R_{1A} is as defined in any one of paragraphs (131) or (132) above; and
R_{1B} is as defined in any one of paragraphs (133) or (134) above.

In a particular group of compounds of formula Is:
L is as defined in any one of paragraphs (12) or (13) above;
R_{L} is as defined in any one of paragraphs (18) or (19) above;
R₁ is as defined in any one of paragraphs (90) or (91) above;
R_{1A} is as defined in any one of paragraphs (131) or (132) above; and
R_{1B} is as defined in any one of paragraphs (133) or (134) above.

In a particular group of compounds of formula Is:
L is as defined in paragraph (13) above;
R_{L} is as defined in paragraph (19) above;
R₁ is as defined in paragraph (91) above;
R_{1A} is as defined in paragraph (132) above; and
R_{1B} is as defined in paragraph (134) above.

In a particular group of compounds of the invention, the compound is a compound of formula I defined herein in which R_{R} is as defined in paragraph (81) above, i.e. the compounds have the formula It shown below, or a pharmaceutically acceptable salt thereof:

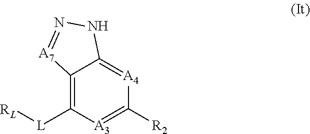

wherein A₃, A₄, A₇, L, R_{L} and R₂ each have any one of the definitions set out herein.

In a particular group of compounds of formula It:
A₃ is as defined in any one of paragraphs (135), (136) or (137) above;
A₄ is as defined in any one of paragraphs (138), (139) or (140) above;
A₇ is as defined in any one of paragraphs (143), (144) or (145) above;
L is as defined in any one of paragraphs (1) to (13) above;
R_{L} is as defined in any one of paragraphs (14) to (19) above; and
R₂ is as defined in any one of paragraphs (146) to (153) above;

In a particular group of compounds of formula It:
A₃ is as defined in any one of paragraphs (135), (136) or (137) above;
A₄ is as defined in any one of paragraphs (138), (139) or (140) above;
A₇ is as defined in any one of paragraphs (143), (144) or (145) above;
L is as defined in any one of paragraphs (4) to (13) above;
R_{L} is as defined in any one of paragraphs (15) to (19) above; and
R₂ is as defined in any one of paragraphs (148) to (153) above.

In a particular group of compounds of formula It:
A₃ is as defined in any one of paragraphs (135), (136) or (137) above;
A₄ is as defined in any one of paragraphs (138), (139) or (140) above;
A₇ is as defined in any one of paragraphs (143), (144) or (145) above;
L is as defined in any one of paragraphs (6) to (13) above;
R_{L} is as defined in any one of paragraphs (16) to (19) above; and
R₂ is as defined in any one of paragraphs (150) to (153) above.

In a particular group of compounds of formula It:
A₃ is as defined in any one of paragraphs (135), (136) or (137) above;
A₄ is as defined in any one of paragraphs (138), (139) or (140) above;
A₇ is as defined in any one of paragraphs (143), (144) or (145) above;
L is as defined in any one of paragraphs (8) to (13) above;
R_{L} is as defined in any one of paragraphs (17) to (19) above; and
R₂ is as defined in any one of paragraphs (151) to (153) above.

In a particular group of compounds of formula It:
A₃ is as defined in any one of paragraphs (135), (136) or (137) above;
A₄ is as defined in any one of paragraphs (138), (139) or (140) above;
A₇ is as defined in any one of paragraphs (143), (144) or (145) above;
L is as defined in any one of paragraphs (10) to (13) above;
R_{L} is as defined in any one of paragraphs (18) or (19) above; and
R₂ is as defined in any one of paragraphs (152) or (153) above.

In a particular group of compounds of formula It:
A₃ is as defined in paragraph (135) above;
A₄ is as defined in paragraph (138) above;
A₇ is as defined in any one of paragraphs (143) or (145) above;
L is as defined in any one of paragraphs (12) or (13) above;
R_{L} is as defined in any one of paragraphs (18) or (19) above; and
R₂ is as defined in any one of paragraphs (152) or (153) above.

In a particular group of compounds of formula It:
A₃ is as defined in paragraph (135) above;
A₄ is as defined in paragraph (138) above;
A₇ is as defined in any one of paragraphs (143) or (145) above;
L is as defined in paragraph (13) above;
R_{L} is as defined in any paragraph (19) above; and
R₂ is as defined in paragraph (153) above.

In a particular group of compounds of the invention, the compound is a compound of formula I defined herein in which R_{R} is as defined in paragraph (81) above, A₃ is as defined in paragraph (135) above, A₄ is as defined in paragraph (139) above and A₅ and A₆ are as defined in paragraph (141) above, i.e. the compounds have the formula Iu shown below, or a pharmaceutically acceptable salt thereof:

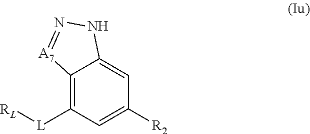

wherein A₇, L, R_{L} and R₂ each have any one of the definitions set out herein.

In a particular group of compounds of formula Iu:
A₇ is as defined in any one of paragraphs (143), (144) or (145) above;
L is as defined in any one of paragraphs (1) to (13) above;
R_{L} is as defined in any one of paragraphs (14) to (19) above; and
R₂ is as defined in any one of paragraphs (146) to (153) above;

In a particular group of compounds of formula Iu:
A₇ is as defined in any one of paragraphs (143), (144) or (145) above;
L is as defined in any one of paragraphs (4) to (13) above;
R_{L} is as defined in any one of paragraphs (15) to (19) above; and
R₂ is as defined in any one of paragraphs (148) to (153) above.

In a particular group of compounds of formula Iu:
A₇ is as defined in any one of paragraphs (143), (144) or (145) above;
L is as defined in any one of paragraphs (6) to (13) above;
R_{L} is as defined in any one of paragraphs (16) to (19) above; and
R₂ is as defined in any one of paragraphs (150) to (153) above.

In a particular group of compounds of formula Iu:
A₇ is as defined in any one of paragraphs (143), (144) or (145) above;
L is as defined in any one of paragraphs (8) to (13) above;
R_{L} is as defined in any one of paragraphs (17) to (19) above; and
R₂ is as defined in any one of paragraphs (151) to (153) above.

In a particular group of compounds of formula Iu:
A₇ is as defined in any one of paragraphs (143), (144) or (145) above;
L is as defined in any one of paragraphs (10) to (13) above;
R_{L} is as defined in any one of paragraphs (18) or (19) above; and
R₂ is as defined in any one of paragraphs (152) or (153) above.

In a particular group of compounds of formula Iu:
A₇ is as defined in any one of paragraphs (143) or (145) above;
L is as defined in any one of paragraphs (12) or (13) above;
R_{L} is as defined in any one of paragraphs (18) or (19) above; and
R₂ is as defined in any one of paragraphs (152) or (153) above.

In a particular group of compounds of formula Iu:
A₇ is as defined in any one of paragraphs (143) or (145) above;
L is as defined in paragraph (13) above;
R_{L} is as defined in any paragraph (19) above; and
R₂ is as defined in paragraph (153) above.

Compounds of the present invention include any of the compounds described in the example section of the present application, or a pharmaceutically acceptable salt, hydrate or solvate thereof, and, in particular, any of the following:
3-(((2-Chloro-[1,1′-biphenyl]-4-yl)methyl)amino)-N-(3-((6-(pyridin-4-yl)-1H-indazol-4-yl)amino)propyl)propanamide;
4-((3-(3-(((2-chloro-[1,1′-biphenyl]-4-yl)methyl)amino)propanamido)propyl)amino)-1H-indazole-6-carboxamide;
N-(3-((6-(1H-pyrazol-4-yl)-1H-indazol-4-yl)amino)propyl)-3-(((2-chloro-[1,1′-biphenyl]-4-yl)methyl)amino)propanamide;
N-(3-((6-(1H-1,2,4-triazol-1-yl)-1H-indazol-4-yl)amino)propyl)-3-(((2-chloro-[1,1′-biphenyl]-4-yl)methyl)amino)propanamide;
3-((3-Chloro-4-(trifluoromethoxy)benzyl)amino)-N-(3-((6-(pyridin-4-yl)-1H-indazol-4-yl)amino)propyl)propanamide;
3-(((2-chloro-[1,1′-biphenyl]-4-yl)methyl)amino)-N-(3-((6-(pyridazin-4-yl)-1H-indazol-4-yl)amino)propyl)propanamide;
3-(((2-chloro-[1,1′-biphenyl]-4-yl)methyl)amino)-N-(3-((6-(3-cyano-1H-pyrazol-4-yl)-1H-indazol-4-yl)amino)propyl)propanamide;
3-(((2-chloro-[1,1′-biphenyl]-4-yl)methyl)amino)-N-(3-((6-(pyridin-4-yl)-1H-benzo[d][1,2,3]triazol-4-yl)amino)propyl)propanamide;
3-(((2-chloro-[1,1′-biphenyl]-4-yl)methyl)amino)-N-(3-((6-(2-methyl-1H-imidazol-4-yl)-1H-indazol-4-yl)amino)propyl)propanamide;
3-(((2-chloro-[1,1′-biphenyl]-4-yl)methyl)amino)-N-(3-((6-cyano-1H-indazol-4-yl)amino)propyl)propanamide;
N-(3-((6-(4H-1,2,4-triazol-4-yl)-1H-indazol-4-yl)amino)propyl)-3-(((2-chloro-[1,1′-biphenyl]-4-yl)methyl)amino)propanamide;
N-(6-(4H-1,2,4-triazol-4-yl)-1H-indazol-4-yl)-3-(3-(((2-chloro-[1,1′-biphenyl]-4-yl)methyl)amino)propanamido)propanamide;
N-(3-((6-(4H-1,2,4-triazol-4-yl)-1H-indazol-4-yl)amino)propyl)-3-((3-chlorobenzyl)amino)propanamide;
N-(2-(4-(2-(((2-chloro-[1,1′-biphenyl]-4-yl)methyl)amino)ethyl)-1H-1,2,3-triazol-1-yl)ethyl)-6-(4H-1,2,4-triazol-4-yl)-1H-indazol-4-amine;
N-(6-(4H-1,2,4-triazol-4-yl)-1H-indazol-4-yl)-2-(2-(2-((3-chloro-4-(trifluoromethoxy)benzyl)amino)ethyl)oxazol-5-yl)acetamide;
N-(2-(5-(2-((3-chloro-4-(trifluoromethoxy)benzyl)amino)ethyl)-4H-1,2,4-triazol-3-yl)ethyl)-6-(4H-1,2,4-triazol-4-yl)-1H-indazol-4-amine;
N-(6-(4H-1,2,4-triazol-4-yl)-1H-indazol-4-yl)-2-((4-((3-chloro-4-(trifluoromethoxy)benzyl)amino)butyl)amino)acetamide;
N-(6-(4H-1,2,4-triazol-4-yl)-1H-indazol-4-yl)-3-(3-((3-chloro-4-(trifluoromethoxy)benzyl)amino)propanamido)propanamide;
N-(3-((6-(4H-1,2,4-triazol-4-yl)-1H-indazol-4-yl)oxy)propyl)-3-((3-chloro-4-(trifluoromethoxy)benzyl)amino)propanamide;
N-(3-((6-(4H-1,2,4-triazol-4-yl)-1H-indazol-4-yl)amino)propyl)-3-((3-chloro-4-(trifluoromethoxy)benzyl)amino)-N-methylpropanamide;
3-(((2-Chloro-[1,1′-biphenyl]-4-yl)methyl)amino)-N-(3-((6-(2-methylpyridin-4-yl)-1H-indazol-4-yl)amino)propyl)propanamide;
3-(((2-Chloro-[1,1′-biphenyl]-4-yl)methyl)amino)-N-(3-((6-(2-methoxypyridin-4-yl)-1H-indazol-4-yl)amino)propyl)propanamide;
N-(2-(4-(((6-chloro-1H-benzo[d]imidazol-2-yl)methyl)amino)butoxy)ethyl)-6-(4H-1,2,4-triazol-4-yl)-1H-indazol-4-amine;
N1-(2-((6-(4H-1,2,4-triazol-4-yl)-1H-indazol-4-yl)amino)ethyl)-N4-(3-chloro-4-(trifluoromethoxy)benzyl)butane-1,4-diamine;
N-(3-((6-(2H-1,2,3-triazol-4-yl)-1H-indazol-4-yl)amino)propyl)-3-(((2-chloro-[1,1′-biphenyl]-4-yl)methyl)amino)propanamide;
3-(((2-chloro-[1,1′-biphenyl]-4-yl)methyl)amino)-N-(3-((6-(pyridin-3-yl)-1H-indazol-4-yl)amino)propyl)propanamide;
3-(((2-chloro-[1,1′-biphenyl]-4-yl)methyl)amino)-N-(3-((6-(3-methyl-1H-pyrazol-4-yl)-1H-indazol-4-yl)amino)propyl)propanamide;
3-(((2-chloro-[1,1′-biphenyl]-4-yl)methyl)amino)-N-(3-((6-(isoxazol-4-yl)-1H-indazol-4-yl)amino)propyl)propanamide;
3-(((2-chloro-[1,1′-biphenyl]-4-yl)methyl)amino)-N-(3-((6-(4-methyl-1H-imidazol-1-yl)-1H-indazol-4-yl)amino)propyl)propanamide;
4-(4-((3-(3-(((2-chloro-[1,1′-biphenyl]-4-yl)methyl)amino)propanamido)propyl)amino)-1H-indazol-6-yl)pyridine 1-oxide;
3-((3-chloro-4-(trifluoromethoxy)benzyl)amino)-N-(3-((6-(2-oxo-1,2-dihydropyridin-4-yl)-1H-indazol-4-yl)amino)propyl)propanamide;
N-(3-((6-(1,2,3-thiadiazol-5-yl)-1H-indazol-4-yl)amino)propyl)-3-((3-chloro-4-(trifluoromethoxy)benzyl)amino)propanamide;
N-(3-((6-(4H-1,2,4-triazol-4-yl)-1H-indazol-4-yl)amino)propyl)-3-((3-chloro-4-(trifluoromethoxy)benzyl)amino)propanamide;
N-(3-((6-(4H-1,2,4-triazol-4-yl)-1H-indazol-4-yl)amino)propyl)-3-(((4-chloro-5-phenyl-1H-pyrazol-3-yl)methyl)amino)propanamide;
N-(3-((6-(4H-1,2,4-triazol-4-yl)-1H-indazol-4-yl)amino)propyl)-3-((3-(hydroxymethyl)benzyl)amino)propanamide;
N-(3-((6-(4H-1,2,4-triazol-4-yl)-1H-indazol-4-yl)amino)propyl)-3-(((6-chloro-1-methyl-1H-indol-2-yl)methyl)amino)propanamide;
N-(3-((6-(4H-1,2,4-triazol-4-yl)-1H-indazol-4-yl)amino)propyl)-3-(((6-chloro-1H-indol-2-yl)methyl)amino)propanamide;
N-(3-((6-(4H-1,2,4-triazol-4-yl)-1H-indazol-4-yl)amino)propyl)-3-(((1-methyl-5-(trifluoromethyl)-1H-indol-2-yl)methyl)amino)propanamide;
N-(3-((6-(4H-1,2,4-triazol-4-yl)-1H-indazol-4-yl)amino)propyl)-3-((3-cyano-4-cyclobutoxybenzyl)amino)propanamide;
N-(3-((6-(4H-1,2,4-triazol-4-yl)-1H-indazol-4-yl)amino)propyl)-3-((3-(2-hydroxyethyl)benzyl)amino)propanamide;
N-(3-((6-(4H-1,2,4-triazol-4-yl)-1H-indazol-4-yl)amino)propyl)-3-((4-cyclobutoxy-3-(hydroxymethyl)benzyl)amino)propanamide;
N-(3-((6-(4H-1,2,4-triazol-4-yl)-1H-indazol-4-yl)amino)propyl)-3-((3-(2-hydroxyethoxy)benzyl)amino)propanamide;
N-(3-((6-(4H-1,2,4-triazol-4-yl)-1H-indazol-4-yl)amino)propyl)-3-((3-chloro-5-(hydroxymethyl)benzyl)amino)propanamide;
N-(3-((6-(4H-1,2,4-triazol-4-yl)-1H-indazol-4-yl)amino)propyl)-3-((3-(hydroxymethyl)-4-(trifluoromethoxy)benzyl)amino)propanamide;
N-(3-((6-(4H-1,2,4-triazol-4-yl)-1H-indazol-4-yl)amino)propyl)-3-((3-cyano-4-cyclopropylbenzyl)amino)propanamide;
N-(3-((6-(4H-1,2,4-triazol-4-yl)-1H-indazol-4-yl)amino)propyl)-3-((3-cyano-4-(trifluoromethoxy)benzyl)amino)propanamide;
N-(3-((6-(4H-1,2,4-triazol-4-yl)-1H-indazol-4-yl)amino)propyl)-3-((3-(cyanomethyl)benzyl)amino)propanamide;
N-(6-(4H-1,2,4-triazol-4-yl)-1H-indazol-4-yl)-2-(4-((3-chloro-4-(trifluoromethoxy)benzyl)amino)butoxy)acetamide; 6-(4H-1,2,4-triazol-4-yl)-N-(2-(4-(((5-(trifluoromethyl)-1H-indol-2-yl)methyl)amino)butoxy)ethyl)-1H-indazol-4-amine;
N-(2-(4-(((6-chloro-1-methyl-1H-indol-2-yl)methyl)amino)butoxy)ethyl)-6-(4H-1,2,4-triazol-4-yl)-1H-indazol-4-amine;
N-(2-(4-(((1-methyl-5-(trifluoromethyl)-1H-indol-2-yl)methyl)amino)butoxy)ethyl)-6-(4H-1,2,4-triazol-4-yl)-1H-indazol-4-amine;
N-(2-(4-(((6-chloro-1H-indol-2-yl)methyl)amino)butoxy)ethyl)-6-(4H-1,2,4-triazol-4-yl)-1H-indazol-4-amine;
N-(2-(4-(((5-cyclobutoxy-1H-indol-2-yl)methyl)amino)butoxy)ethyl)-6-(4H-1,2,4-triazol-4-yl)-1H-indazol-4-amine;
N-((5-cyclobutoxy-1H-indol-2-yl)methyl)-4-(2-((6-(isoxazol-4-yl)-1H-indazol-4-yl)oxy)ethoxy)butan-1-amine;
N-((5-cyclobutoxy-1H-indol-2-yl)methyl)-4-(2-((6-(pyridazin-4-yl)-1H-indazol-4-yl)oxy)ethoxy)butan-1-amine;
4-(2-((6-(pyridazin-4-yl)-1H-indazol-4-yl)oxy)ethoxy)-N-((5-(trifluoromethyl)-1H-indol-2-yl)methyl)butan-1-amine;
N-(2-(4-(((5-cyclobutoxy-1H-indol-2-yl)methyl)amino)butoxy)ethyl)-6-(pyridazin-4-yl)-1H-indazol-4-amine;
N-(3-((6-(4H-1,2,4-triazol-4-yl)-1H-indazol-4-yl)amino)propyl)-3-(((2-chloro-2′-(hydroxymethyl)-[1,1′-biphenyl]-4-yl)methyl)amino)propanamide;
4-((3-(3-(((2-chloro-[1,1′-biphenyl]-4-yl)methyl)amino)propanamido)propyl)amino)-1H-indazole-6-carboxylic acid;
4-((4-((4-(((2-chloro-[1,1′-biphenyl]-4-yl)methyl)amino)butyl)amino)butyl)amino)-1H-indazole-6-carboxylic acid;
N-(3-((1H-indazol-4-yl)amino)propyl)-3-(((2-chloro-[1,1′-biphenyl]-4-yl)methyl)amino)propanamide;
4-((3-(3-(((2-chloro-[1,1′-biphenyl]-4-yl)methyl)amino)propanamido)propyl)amino)-N,N-dimethyl-1H-indazole-6-carboxamide;
3-(((2-chloro-[1,1′-biphenyl]-4-yl)methyl)amino)-N-(3-((6-(hydroxymethyl)-1H-indazol-4-yl)amino)propyl)propanamide;
N-(2-(2-(2-((3-chloro-4-(trifluoromethoxy)benzyl)amino)ethyl)oxazol-5-yl)ethyl)-6-(4H-1,2,4-triazol-4-yl)-1H-indazol-4-amine;
N-(6-(4H-1,2,4-triazol-4-yl)-1H-indazol-4-yl)-2-(5-(2-((3-chloro-4-(trifluoromethoxy)benzyl)amino)ethyl)-4H-1,2,4-triazol-3-yl)acetamide;
N-(6-(4H-1,2,4-triazol-4-yl)-1H-indazol-4-yl)-3-((3-((3-chloro-4-(trifluoromethoxy)benzyl)amino)propyl)amino)propanamide;
N1-(3-((6-(4H-1,2,4-triazol-4-yl)-1H-indazol-4-yl)oxy)propyl)-N3-(3-chloro-4-(trifluoromethoxy)benzyl)propane-1,3-diamine;
N-(2-(4-(((6-chloro-1-methyl-1H-benzo[d]imidazol-2-yl)methyl)amino)butoxy)ethyl)-6-(4H-1,2,4-triazol-4-yl)-1H-indazol-4-amine;
3-(3-((6-(4H-1,2,4-triazol-4-yl)-1H-indazol-4-yl)oxy)propoxy)-N-(3-chloro-4-(trifluoromethoxy)benzyl)propan-1-amine;
N-(6-(4H-1,2,4-triazol-4-yl)-1H-indazol-4-yl)-3-(3-((3-chloro-4-(trifluoromethoxy)benzyl)amino)propoxy)propanamide;
3-(((2-chloro-[1,1′-biphenyl]-4-yl)methyl)amino)-N-(3-((6-(3-methyl-4H-1,2,4-triazol-4-yl)-1H-indazol-4-yl)amino)propyl)propanamide;
N1-(2-((6-(4H-1,2,4-triazol-4-yl)-1H-indazol-4-yl)amino)ethyl)-N4-(3-chloro-4-(trifluoromethoxy)benzyl)-N1-methylbutane-1,4-diamine;
3-(((2-chloro-[1,1′-biphenyl]-4-yl)methyl)amino)-N-(3-((6-(pyrimidin-5-yl)-1H-indazol-4-yl)amino)propyl)propanamide;
3-(((2-chloro-[1,1′-biphenyl]-4-yl)methyl)amino)-N-(3-((6-(2-cyanopyridin-4-yl)-1H-indazol-4-yl)amino)propyl)propanamide;
3-(((2-chloro-[1,1′-biphenyl]-4-yl)methyl)amino)-N-(3-((6-(pyrimidin-4-yl)-1H-indazol-4-yl)amino)propyl)propanamide;
3-(((2-chloro-[1,1′-biphenyl]-4-yl)methyl)amino)-N-(3-((6-(3-methoxy-1H-pyrazol-4-yl)-1H-indazol-4-yl)amino)propyl)propanamide;
N-(3-((6-(4H-1,2,4-triazol-4-yl)-1H-indazol-4-yl)amino)propyl)-3-((4-cyclopropyl-3-(hydroxymethyl)benzyl)amino)propanamide;
3-(((2-chloro-[1,1′-biphenyl]-4-yl)methyl)amino)-N-(3-((6-morpholino-1H-indazol-4-yl)amino)propyl)propanamide;
N-(3-((6-(2H-tetrazol-5-yl)-1H-indazol-4-yl)amino)propyl)-3-(((2-chloro-[1,1′-biphenyl]-4-yl)methyl)amino)propanamide;
3-(((2-chloro-[1,1′-biphenyl]-4-yl)methyl)amino)-N-(3-((6-(2-methyl-1H-imidazol-1-yl)-1H-indazol-4-yl)amino)propyl)propanamide;
3-(((2-chloro-[1,1′-biphenyl]-4-yl)methyl)amino)-N-(3-((6-(oxetan-3-yl)-1H-indazol-4-yl)amino)propyl)propanamide;
3-(((2-chloro-[1,1′-biphenyl]-4-yl)methyl)amino)-N-(3-((6-(tetrahydrofuran-3-yl)-1H-indazol-4-yl)amino)propyl)propanamide;
N-(3-((5-(4H-1,2,4-triazol-4-yl)-1H-indazol-7-yl)amino)propyl)-3-((3-chloro-4-(trifluoromethoxy)benzyl)amino)propanamide; methyl 4-((2-(4-(((5-(trifluoromethyl)-1H-indol-2-yl)methyl)amino)butoxy)ethyl)amino)-1H-indazole-6-carboxylate;
4-((2-(4-(((5-(trifluoromethyl)-1H-indol-2-yl)methyl)amino)butoxy)ethyl)amino)-1H-indazole-6-carboxylic acid;
6-(3-methyl-4H-1,2,4-triazol-4-yl)-N-(2-(4-(((5-(trifluoromethyl)-1H-indol-2-yl)methyl)amino)butoxy)ethyl)-1H-indazol-4-amine;
6-(2-methoxypyridin-4-yl)-N-(2-(4-(((5-(trifluoromethyl)-1H-indol-2-yl)methyl)amino)butoxy)ethyl)-1H-indazol-4-amine;
methyl 3-(4-((2-(4-(((5-(trifluoromethyl)-1H-indol-2-yl)methyl)amino)butoxy)ethyl)amino)-1H-indazol-6-yl)propanoate;
3-(4-((2-(4-(((5-(trifluoromethyl)-1H-indol-2-yl)methyl)amino)butoxy)ethyl)amino)-1H-indazol-6-yl)propanoic acid;
3-(((2-chloro-[1,1′-biphenyl]-4-yl)methyl)amino)-N-(3-((6-(tetrahydro-2H-pyran-4-yl)-1H-indazol-4-yl)amino)propyl)propanamide;
3-(((2-chloro-[1,1′-biphenyl]-4-yl)methyl)amino)-N-(3-((6-(4-methyl-1H-imidazol-5-yl)-1H-indazol-4-yl)amino)propyl)propanamide;
6-(3-methoxy-1H-pyrazol-4-yl)-N-(2-(4-(((5-(trifluoromethyl)-1H-indol-2-yl)methyl)amino)butoxy)ethyl)-1H-indazol-4-amine;
6-(pyridazin-4-yl)-N-(2-(4-(((5-(trifluoromethyl)-1H-indol-2-yl)methyl)amino)butoxy)ethyl)-1H-indazol-4-amine;
N-(2-(4-(((5-(trifluoromethyl)-1H-indol-2-yl)methyl)amino)butoxy)ethyl)-1H-indazol-4-amine;
6-(pyrimidin-4-yl)-N-(2-(4-(((5-(trifluoromethyl)-1H-indol-2-yl)methyl)amino)butoxy)ethyl)-1H-indazol-4-amine;
4-((2-(4-(((5-(trifluoromethyl)-1H-indol-2-yl)methyl)amino)butoxy)ethyl)amino)-1H-indazole-6-carbonitrile;
6-(isoxazol-4-yl)-N-(2-(4-(((5-(trifluoromethyl)-1H-indol-2-yl)methyl)amino)butoxy)ethyl)-1H-indazol-4-amine;
N-(2-(4-(((5-cyclobutoxy-1H-indol-2-yl)methyl)amino)butoxy)ethyl)-6-(isoxazol-4-yl)-1H-indazol-4-amine;
N-(2-(4-((3-chloro-4-(trifluoromethoxy)benzyl)amino)butoxy)ethyl)-6-(pyridazin-4-yl)-1H-benzo[d][1,2,3]triazol-4-amine;
6-(pyridazin-4-yl)-N-(2-(4-(((5-(trifluoromethyl)-1H-indol-2-yl)methyl)amino)butoxy)ethyl)-1H-benzo[d][1,2,3]triazol-4-amine;
4-(2-((6-(pyridazin-4-yl)-1H-benzo[d][1,2,3]triazol-4-yl)oxy)ethoxy)-N-((5-(trifluoromethyl)-1H-indol-2-yl)methyl)butan-1-amine;
N-(3-chloro-4-(trifluoromethoxy)benzyl)-4-(2-((6-(pyridazin-4-yl)-1H-benzo[d][1,2,3]triazol-4-yl)oxy)ethoxy)butan-1-amine;
2-(3-(((4-((1-(6-(4H-1,2,4-triazol-4-yl)-1H-indazol-4-yl)azetidin-3-yl)oxy)butyl)amino)methyl)-5-fluorophenyl)acetonitrile;
(3-(((4-((1-(6-(4H-1,2,4-triazol-4-yl)-1H-indazol-4-yl)azetidin-3-yl)oxy)butyl)amino)methyl)-5-(trifluoromethyl)phenyl)methanol;
2-(3-(((4-((1-(6-(4H-1,2,4-triazol-4-yl)-1H-indazol-4-yl)azetidin-3-yl)oxy)butyl)amino)methyl)-5-(trifluoromethyl)phenyl)acetonitrile;
2-(3-(((4-((1-(6-(4H-1,2,4-triazol-4-yl)-1H-indazol-4-yl)azetidin-3-yl)oxy)butyl)amino)methyl)-5-methylphenyl)acetonitrile;
N-(3-chloro-4-(trifluoromethoxy)benzyl)-4-((1-(6-(pyridazin-4-yl)-1H-indazol-4-yl)azetidin-3-yl)oxy)butan-1-amine;
N-(3-fluoro-4-(trifluoromethoxy)benzyl)-4-((1-(6-(pyridazin-4-yl)-1H-indazol-4-yl)azetidin-3-yl)oxy)butan-1-amine;
N-((5-cyclobutoxy-1H-indol-2-yl)methyl)-4-((1-(6-(pyridazin-4-yl)-1H-indazol-4-yl)azetidin-3-yl)oxy)butan-1-amine;
(3-fluoro-5-(((4-((1-(6-(pyridazin-4-yl)-1H-indazol-4-yl)azetidin-3-yl)oxy)butyl)amino)methyl)phenyl)methanol;
(3-chloro-5-(((4-((1-(6-(pyridazin-4-yl)-1H-indazol-4-yl)azetidin-3-yl)oxy)butyl)amino)methyl)phenyl)methanol;
(3-(((4-((1-(6-(pyridazin-4-yl)-1H-indazol-4-yl)azetidin-3-yl)oxy)butyl)amino)methyl)-5-(trifluoromethyl)phenyl)methanol;
2-(3-fluoro-5-(((4-((1-(6-(pyridazin-4-yl)-1H-indazol-4-yl)azetidin-3-yl)oxy)butyl)amino)methyl)phenyl)acetonitrile;
2-(3-(((4-((1-(6-(pyridazin-4-yl)-1H-indazol-4-yl)azetidin-3-yl)oxy)butyl)amino)methyl)-5-(trifluoromethyl)phenyl)acetonitrile;
2-(3-methyl-5-(((4-((1-(6-(pyridazin-4-yl)-1H-indazol-4-yl)azetidin-3-yl)oxy)butyl)amino)methyl)phenyl)acetonitrile;
4-(2-((6-(4H-1,2,4-triazol-4-yl)-1H-benzo[d][1,2,3]triazol-4-yl)oxy)ethoxy)-N-((5-(trifluoromethyl)-1H-indol-2-yl)methyl)butan-1-amine;
4-(2-((6-(4H-1,2,4-triazol-4-yl)-1H-indazol-4-yl)oxy)ethoxy)-N-((5-(trifluoromethyl)-1H-indol-2-yl)methyl)butan-1-amine;
4-(2-((6-(4H-1,2,4-triazol-4-yl)-1H-indazol-4-yl)oxy)ethoxy)-N-((5-cyclobutoxy-1H-indol-2-yl)methyl)butan-1-amine;
N-((5-cyclobutoxy-1H-indol-2-yl)methyl)-4-((1-(6-(isoxazol-4-yl)-1H-indazol-4-yl)azetidin-3-yl)oxy)butan-1-amine;
(3-(((4-((1-(6-(isoxazol-4-yl)-1H-indazol-4-yl)azetidin-3-yl)oxy)butyl)amino)methyl)-5-(trifluoromethyl)phenyl)methanol;
2-(3-(((4-((1-(6-(isoxazol-4-yl)-1H-indazol-4-yl)azetidin-3-yl)oxy)butyl)amino)methyl)-5-methylphenyl)acetonitrile;
2-(3-chloro-5-(((4-((1-(6-(isoxazol-4-yl)-1H-indazol-4-yl)azetidin-3-yl)oxy)butyl)amino)methyl)phenyl)acetonitrile;
4-(2-((6-(4H-1,2,4-triazol-4-yl)-1H-benzo[d][1,2,3]triazol-4-yl)oxy)ethoxy)-N-(3-chloro-4-(trifluoromethoxy)benzyl)butan-1-amine;
4-((1-(6-(4H-1,2,4-triazol-4-yl)-1H-indazol-4-yl)azetidin-3-yl)oxy)-N-((5-cyclobutoxy-1H-indol-2-yl)methyl)butan-1-amine;
(3-(((4-((1-(6-(4H-1,2,4-triazol-4-yl)-1H-indazol-4-yl)azetidin-3-yl)oxy)butyl)amino)methyl)-5-fluorophenyl)methanol;
N-(2-(4-((3-chloro-4-(trifluoromethoxy)benzyl)amino)butoxy)ethyl)-6-(4H-1,2,4-triazol-4-yl)-1H-benzo[d][1,2,3]triazol-4-amine;
N-(2-(4-((3-chloro-4-(trifluoromethoxy)benzyl)amino)butoxy)ethyl)-6-(isoxazol-4-yl)-1H-benzo[d][1,2,3]triazol-4-amine;
4-((1-(6-(4H-1,2,4-triazol-4-yl)-1H-indazol-4-yl)azetidin-3-yl)oxy)-N-((5-(trifluoromethyl)-1H-indol-2-yl)methyl)butan-1-amine;
4-((1-(6-(4H-1,2,4-triazol-4-yl)-1H-indazol-4-yl)azetidin-3-yl)oxy)-N-(3-fluoro-4-(trifluoromethoxy)benzyl)butan-1-amine;
(3-(((4-((1-(6-(4H-1,2,4-triazol-4-yl)-1H-indazol-4-yl)azetidin-3-yl)oxy)butyl)amino)methyl)-5-chlorophenyl)methanol;
2-(3-(((4-((1-(6-(4H-1,2,4-triazol-4-yl)-1H-indazol-4-yl)azetidin-3-yl)oxy)butyl)amino)methyl)-5-chlorophenyl)acetonitrile;
4-((1-(6-(4H-1,2,4-triazol-4-yl)-1H-indazol-4-yl)azetidin-3-yl)oxy)-N-(3-chloro-4-(trifluoromethoxy)benzyl)butan-1-amine;
2-(3-chloro-5-(((4-((1-(6-(pyridazin-4-yl)-1H-indazol-4-yl)azetidin-3-yl)oxy)butyl)amino)methyl)phenyl)acetonitrile;
2-(3-(((4-((1-(6-(pyridazin-4-yl)-1H-indazol-4-yl)azetidin-3-yl)oxy)butyl)amino)methyl)-5-(trifluoromethoxy)phenyl)acetonitrile;
N-(3-fluoro-4-(trifluoromethoxy)benzyl)-4-((1-(6-(isoxazol-4-yl)-1H-indazol-4-yl)azetidin-3-yl)oxy)butan-1-amine;
(3-chloro-5-(((4-((1-(6-(isoxazol-4-yl)-1H-indazol-4-yl)azetidin-3-yl)oxy)butyl)amino)methyl)phenyl)methanol;
N-(3-chloro-4-(trifluoromethoxy)benzyl)-4-((1-(6-(isoxazol-4-yl)-1H-indazol-4-yl)azetidin-3-yl)oxy)butan-1-amine;
(3-fluoro-5-(((4-((1-(6-(isoxazol-4-yl)-1H-indazol-4-yl)azetidin-3-yl)oxy)butyl)amino)methyl)phenyl)methanol;
4-((1-(6-(isoxazol-4-yl)-1H-indazol-4-yl)azetidin-3-yl)oxy)-N-((5-(trifluoromethyl)-1H-indol-2-yl)methyl)butan-1-amine;
4-((1-(6-(pyridazin-4-yl)-1H-indazol-4-yl)azetidin-3-yl)oxy)-N-((5-(trifluoromethyl)-1H-indol-2-yl)methyl)butan-1-amine;
2-(3-(((4-(2-((6-(4H-1,2,4-triazol-4-yl)-1H-benzo[d][1,2,3]triazol-4-yl)amino)ethoxy)butyl)amino)methyl)-5-(trifluoromethoxy)phenyl)acetonitrile;
6-(4H-1,2,4-triazol-4-yl)-N-(2-(4-(((5-(trifluoromethyl)-1H-indol-2-yl)methyl)amino)butoxy)ethyl)-1H-benzo[d][1,2,3]triazol-4-amine;
6-(isoxazol-4-yl)-N-(2-(4-(((5-(trifluoromethyl)-1H-indol-2-yl)methyl)amino)butoxy)ethyl)-1H-benzo[d][1,2,3]triazol-4-amine;
4-(2-((6-(isoxazol-4-yl)-1H-indazol-4-yl)oxy)ethoxy)-N-((5-(trifluoromethyl)-1H-indol-2-yl)methyl)butan-1-amine;
2-(3-(((4-((1-(6-(isoxazol-4-yl)-1H-indazol-4-yl)azetidin-3-yl)oxy)butyl)amino)methyl)-5-(trifluoromethyl)phenyl)acetonitrile;
2-(3-(((4-((1-(6-(isoxazol-4-yl)-1H-indazol-4-yl)azetidin-3-yl)oxy)butyl)amino)methyl)-5-(trifluoromethoxy)phenyl)acetonitrile;
2-(3-(((4-((1-(6-(4H-1,2,4-triazol-4-yl)-1H-indazol-4-yl)azetidin-3-yl)oxy)butyl)amino)methyl)-5-(trifluoromethoxy)phenyl)acetonitrile;
2-(3-(((4-(2-((6-(4H-1,2,4-triazol-4-yl)-1H-benzo[d][1,2,3]triazol-4-yl)oxy)ethoxy)butyl)amino)methyl)-5-(trifluoromethoxy)phenyl)acetonitrile;
4-((1-(6-(pyridazin-4-yl)-1H-benzo[d][1,2,3]triazol-4-yl)azetidin-3-yl)oxy)-N-((5-(trifluoromethyl)-1H-indol-2-yl)methyl)butan-1-amine;
N-(3-chloro-4-(trifluoromethoxy)benzyl)-4-((1-(6-(pyridazin-4-yl)-1H-benzo[d][1,2,3]triazol-4-yl)azetidin-3-yl)oxy)butan-1-amine;
2-(3-(((4-((1-(6-(pyridazin-4-yl)-1H-benzo[d][1,2,3]triazol-4-yl)azetidin-3-yl)oxy)butyl)amino)methyl)-5-(trifluoromethoxy)phenyl)acetonitrile;
4-((1-(6-(4H-1,2,4-triazol-4-yl)-1H-benzo[d][1,2,3]triazol-4-yl)azetidin-3-yl)oxy)-N-((5-(trifluoromethyl)-1H-indol-2-yl)methyl)butan-1-amine;
4-((1-(6-(4H-1,2,4-triazol-4-yl)-1H-benzo[d][1,2,3]triazol-4-yl)azetidin-3-yl)oxy)-N-(3-chloro-4-(trifluoromethoxy)benzyl)butan-1-amine;
2-(3-(((4-((1-(6-(4H-1,2,4-triazol-4-yl)-1H-benzo[d][1,2,3]triazol-4-yl)azetidin-3-yl)oxy)butyl)amino)methyl)-5-(trifluoromethoxy)phenyl)acetonitrile;
2-(3-(((4-(2-((6-(pyridazin-4-yl)-1H-benzo[d][1,2,3]triazol-4-yl)oxy)ethoxy)butyl)amino)methyl)-5-(trifluoromethoxy)phenyl)acetonitrile;
3-((3-chloro-4-(trifluoromethoxy)benzyl)amino)-N-(3-((6-(5-cyano-1H-pyrazol-4-yl)-1H-indazol-4-yl)amino)propyl)propanamide;
N-(3-((6-(1,3,4-oxadiazol-2-yl)-1H-indazol-4-yl)amino)propyl)-3-((3-chloro-4-(trifluoromethoxy)benzyl)amino)propanamide;
3-((3-chloro-4-(trifluoromethoxy)benzyl)amino)-N-(3-((7-chloro-6-(pyridin-4-yl)-1H-indazol-4-yl)amino)propyl)propanamide;
3-((3-chloro-4-(trifluoromethoxy)benzyl)amino)-N-(3-((6-(5-methyl-1H-pyrazol-4-yl)-1H-indazol-4-yl)amino)propyl)propanamide;
3-((3-chloro-4-(trifluoromethoxy)benzyl)amino)-N-(3-((6-cyano-1H-indazol-4-yl)amino)propyl)propanamide;
N-(3-((6-(1H-1,2,3-triazol-1-yl)-1H-indazol-4-yl)amino)propyl)-3-((3-chloro-4-(trifluoromethoxy)benzyl)amino)propanamide;
3-((3-chloro-4-(trifluoromethoxy)benzyl)amino)-N-(3-((6-(3-fluoropyridin-4-yl)-1H-indazol-4-yl)amino)propyl)propanamide;
N-(3-((1H-indazol-4-yl)amino)propyl)-3-((3-chloro-4-(trifluoromethoxy)benzyl)amino)propanamide;
3-((3-chloro-4-(trifluoromethoxy)benzyl)amino)-N-(3-((6-(oxazol-5-yl)-1H-indazol-4-yl)amino)propyl)propanamide;
3-((3-chloro-4-(trifluoromethoxy)benzyl)amino)-N-(3-((6-(3-methylpyridin-4-yl)-1H-indazol-4-yl)amino)propyl)propanamide;
7-chloro-N-(2-(4-((3,5-difluoro-4-(trifluoromethoxy)benzyl)amino)butoxy)ethyl)-6-(pyridin-4-yl)-1H-indazol-4-amine;
4-((2-(4-((3,5-difluoro-4-(trifluoromethoxy)benzyl)amino)butoxy)ethyl)amino)-1H-indazole-6-carbonitrile;
3-((3-chloro-4-(trifluoromethoxy)benzyl)amino)-N-(3-((6-(6-oxo-1,6-dihydropyridazin-4-yl)-1H-indazol-4-yl)amino)propyl)propanamide;
3-((3-chloro-4-(trifluoromethoxy)benzyl)amino)-N-(3-((6-(3-chloropyridin-4-yl)-1H-indazol-4-yl)amino)propyl)propanamide;
3-((3-chloro-4-(trifluoromethoxy)benzyl)amino)-N-(3-((6-(2-oxo-1,2-dihydropyrimidin-4-yl)-1H-indazol-4-yl)amino)propyl)propanamide;
4-(2-(((6-(4H-1,2,4-triazol-4-yl)-1H-indazol-4-yl)methyl)amino)ethoxy)-N-(3,5-difluoro-4-(trifluoromethoxy)benzyl)butan-1-amine;
N-(3-(((6-(4H-1,2,4-triazol-4-yl)-1H-indazol-4-yl)methyl)amino)propyl)-3-((3-chloro-4-(trifluoromethoxy)benzyl)amino)propanamide;
N-(3-((6-(1,2,4-oxadiazol-5-yl)-1H-indazol-4-yl)amino)propyl)-3-((3-chloro-4-(trifluoromethoxy)benzyl)amino)propanamide;
N-(3-((6-(4H-1,2,4-triazol-3-yl)-1H-indazol-4-yl)amino)propyl)-3-((3-chloro-4-(trifluoromethoxy)benzyl)amino)propanamide;
3-((3-chloro-4-(trifluoromethoxy)benzyl)amino)-N-(3-((6-(oxazol-4-yl)-1H-indazol-4-yl)amino)propyl)propanamide;
N-(2-(4-((3,5-difluoro-4-(trifluoromethoxy)benzyl)amino)butoxy)ethyl)-7-fluoro-6-(pyridin-4-yl)-1H-indazol-4-amine;
3-((3-chloro-4-(trifluoromethoxy)benzyl)amino)-N-(3-((6-(3-cyano-4H-1,2,4-triazol-4-yl)-1H-indazol-4-yl)amino)propyl)propanamide;
3-((3-chloro-4-(trifluoromethoxy)benzyl)amino)-N-(3-((7-methyl-6-(pyridin-4-yl)-1H-indazol-4-yl)amino)propyl)propanamide;
N-(2-(4-((3,5-difluoro-4-(trifluoromethoxy)benzyl)amino)butoxy)ethyl)-7-methyl-6-(pyridin-4-yl)-1H-indazol-4-amine;
3-((3-chloro-4-(trifluoromethoxy)benzyl)amino)-N-(3-((7-fluoro-6-(pyridin-4-yl)-1H-indazol-4-yl)amino)propyl)propanamide;
3-((3-chloro-4-(trifluoromethoxy)benzyl)amino)-N-(3-((7-chloro-6-(4H-1,2,4-triazol-4-yl)-1H-indazol-4-yl)amino)propyl)propanamide;
7-chloro-N-(2-(4-((3,5-difluoro-4-(trifluoromethoxy)benzyl)amino)butoxy)ethyl)-6-(4H-1,2,4-triazol-4-yl)-1H-indazol-4-amine;
N-(3-((6-(1,2,4-thiadiazol-5-yl)-1H-indazol-4-yl)amino)propyl)-3-((3-chloro-4-(trifluoromethoxy)benzyl)amino)propanamide;
N-(3-((6-(2-aminopyridin-4-yl)-1H-indazol-4-yl)amino)propyl)-3-((3-chloro-4-(trifluoromethoxy)benzyl)amino)propanamide;
3-((3-chloro-4-(trifluoromethoxy)benzyl)amino)-N-(3-((6-(pyridin-4-yl)-1H-pyrazolo[3,4-b]pyridin-4-yl)amino)propyl)propanamide;
5-chloro-N-(2-(4-((3,5-difluoro-4-(trifluoromethoxy)benzyl)amino)butoxy)ethyl)-6-(4H-1,2,4-triazol-4-yl)-1H-indazol-4-amine;
N-(3-((6-(6-aminopyridazin-4-yl)-1H-indazol-4-yl)amino)propyl)-3-((3-chloro-4-(trifluoromethoxy)benzyl)amino)propanamide;
N-(3-((6-(6-aminopyridazin-4-yl)-1H-indazol-4-yl)amino)propyl)-3-((3-chloro-4-(trifluoromethoxy)benzyl)amino)propanamide;
N-(3-((6-(1,2,4-oxadiazol-3-yl)-1H-indazol-4-yl)amino)propyl)-3-((3-chloro-4-(trifluoromethoxy)benzyl)amino)propanamide;
N4-(2-(4-((3,5-difluoro-4-(trifluoromethoxy) benzyl)amino) butoxy)ethyl)-N3-methyl-6-(4H-1,2,4-triazol-4-yl)-1H-indazole-3,4-diamine;
3-((3-chloro-4-(trifluoromethoxy)benzyl)amino)-N-(3-((3-(methylamino)-6-(4H-1,2,4-triazol-4-yl)-1H-indazol-4-yl)amino)propyl)propanamide;
(S)-(3-(((5-(2-((6-(4H-1,2,4-triazol-4-yl)-1H-indazol-4-yl)amino)ethoxy)pentan-2-yl)amino)methyl)-5-(trifluoromethoxy)phenyl)methanol;
(S)-5-(4-((2-((4-((3-(hydroxymethyl)-5-(trifluoromethoxy)benzyl)amino)pentyl)oxy)ethyl)amino)-1H-indazol-6-yl)pyridazin-3-ol;
(S)-2-(3-(((5-(2-((6-(6-hydroxypyridazin-4-yl)-1H-indazol-4-yl)amino)ethoxy)pentan-2-yl)amino)methyl)-5-(trifluoromethoxy)phenyl)acetonitrile;
(S)-5-(4-((2-((4-((3-(oxazol-5-ylmethyl)-5-(trifluoromethoxy)benzyl)amino)pentyl)oxy)ethyl)amino)-1H-indazol-6-yl)pyridazin-3-ol;
(S)-3-(((5-(2-((6-(isoxazol-4-yl)-1H-indazol-4-yl)amino)ethoxy)pentan-2-yl)amino)methyl)-5-(trifluoromethoxy) benzamide;
4-((2-(4-((3,5-difluoro-4-(trifluoromethoxy)benzyl)amino)butoxy)ethyl)amino)-1H-indazole-6-carboxylic acid;
2-(4-((2-(4-((3,5-difluoro-4-(trifluoromethoxy)benzyl)amino)butoxy)ethyl)amino)-1H-indazol-6-yl)acetic acid;
5-(4-(2-((3-((3-(2-hydroxyethoxy)-5-(trifluoromethoxy)benzyl)amino)cyclobutyl)methoxy)ethoxy)-1H-indazol-6-yl)pyridazin-3-ol;
5-(4-(2-((3-((3-(hydroxymethyl)-5-(trifluoromethoxy)benzyl)amino)cyclobutyl)methoxy)ethoxy)-1H-indazol-6-yl)pyridazin-3-ol;
3-(4-((2-(4-((3,5-difluoro-4-(trifluoromethoxy)benzyl)amino)butoxy)ethyl)amino)-1H-indazol-6-yl)propanoic acid;
2-(3-(((4-(2-((1H-indazol-4-yl)amino)ethoxy)butyl)amino)methyl)-5-(trifluoromethoxy)phenoxy)ethanol;
(3-(((3-((2-((6-(1,2,3-thiadiazol-5-yl)-1H-indazol-4-yl)amino)ethoxy)methyl)cyclobutyl)amino)methyl)-5-(trifluoromethoxy)phenyl)methanol;
2-(4-((2-(4-((3-(hydroxymethyl)-5-(trifluoromethoxy)benzyl)amino)butoxy)ethyl)amino)-1H-indazol-6-yl)acetonitrile;
2-(3-(((3-((2-((6-(1,2,3-thiadiazol-5-yl)-1H-indazol-4-yl)amino)ethoxy)methyl)cyclobutyl)amino)methyl)-5-(trifluoromethoxy)phenoxy)ethan-1-ol;
2-(4-((2-(4-((3-(hydroxymethyl)-5-(trifluoromethoxy)benzyl)amino)butoxy)ethyl)amino)-1H-indazol-6-yl)-2-methylpropanenitrile;
(S)-(3-(((5-(2-((6-(1,2,3-thiadiazol-5-yl)-1H-indazol-4-yl)amino)ethoxy)pentan-2-yl)amino)methyl)-5-(trifluoromethoxy)phenyl)methanol; and
(R)-(3-(((4-(2-((6-(1,2,3-thiadiazol-5-yl)-1H-indazol-4-yl)amino)propoxy)butyl)amino)methyl)-5-(trifluoromethoxy)phenyl)methanol.

Though the present invention may relate to any compound or particular group of compounds defined herein by way of optional, preferred or suitable features or otherwise in terms of particular embodiments, the present invention may also relate to any compound or particular group of compounds that specifically excludes said optional, preferred or suitable features or particular embodiments.

Suitably, the present invention excludes any individual compounds not possessing the biological activity defined herein.

### Salts and Solvates

The compounds (including final products and intermediates) described herein may be isolated and used per se or may be isolated in the form of a salt, suitably pharmaceutically acceptable salts. It should be understood that the terms “salt(s)” and “salt form(s)” used by themselves or in conjunction with another term or terms encompasses all inorganic and organic salts, including industrially acceptable salts, as defined herein, and pharmaceutically acceptable salts, as defined herein, unless otherwise specified. As used herein, industrially acceptable salts are salts that are generally suitable for manufacturing and/or processing (including purification) as well as for shipping and storage, but may not be salts that are typically administered for clinical or therapeutic use. Industrially acceptable salts may be prepared on a laboratory scale, i.e. multi-gram or smaller, or on a larger scale, i.e. up to and including a kilogram or more.

Pharmaceutically acceptable salts, as used herein, are salts that are generally chemically and/or physically compatible with the other ingredients comprising a formulation, and/or are generally physiologically compatible with the recipient thereof. Pharmaceutically acceptable salts may be prepared on a laboratory scale, i.e. multi-gram or smaller, or on a larger scale, i.e. up to and including a kilogram or more. It should be understood that pharmaceutically acceptable salts are not limited to salts that are typically administered or approved by the FDA or equivalent foreign regulatory body for clinical or therapeutic use in humans. A practitioner of ordinary skill will readily appreciate that some salts are both industrially acceptable as well as pharmaceutically acceptable salts. It should be understood that all such salts, including mixed salt forms, are within the scope of the application.

In one embodiment, the compounds of Formula I and sub-formulae thereof are isolated as pharmaceutically acceptable salts.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulfuric, phosphoric, trifluoroacetic, formic, citric or maleic acid. In addition, a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

In general, salts of the present application can be prepared in situ during the isolation and/or purification of a compound (including intermediates), or by separately reacting the compound (or intermediate) with a suitable organic or inorganic acid or base (as appropriate) and isolating the salt thus formed. The degree of ionisation in the salt may vary from completely ionised to almost non-ionised. In practice, the various salts may be precipitated (with or without the addition of one or more co-solvents and/or anti-solvents) and collected by filtration or the salts may be recovered by evaporation of solvent(s). Salts of the present application may also be formed via a “salt switch” or ion exchange/double displacement reaction, i.e. reaction in which one ion is replaced (wholly or in part) with another ion having the same charge. One skilled in the art will appreciate that the salts may be prepared and/or isolated using a single method or a combination of methods.

Representative salts include, but are not limited to, acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate, trifluoroacetate and the like. Other examples of representative salts include alkali or alkaline earth metal cations such as, but not limited to, sodium, lithium, potassium, calcium, magnesium, and the like, as well as non-toxic ammonium, quaternary ammonium and amine cations including, but not limited to, ammonium, tetramethylammonium, tetraethylammonium, lysine, arginine, benzathine, choline, tromethamine, diolamine, glycine, meglumine, olamine and the like.

Certain compounds of the Formula I and sub-formulae thereof may exist in solvated as well as unsolvated forms such as, for example, hydrated forms. It is to be understood that the invention encompasses all such solvated forms that possess the biological activity described herein.

### Polymorphs

It is also to be understood that certain compounds of the Formula I and sub-formulae thereof may exhibit polymorphism, and that the invention encompasses all such forms that possess the biological activity described herein.

### N-Oxides

Compounds of the Formula I and sub-formulae thereof containing an amine function may also form N-oxides. A reference herein to a compound of the Formula I and sub-formulae thereof that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-Oxides can be formed by treatment of the corresponding amine with an oxidizing agent such as, but not limited to, hydrogen peroxide or a per-acid (e.g. a peroxycarboxylic acid), see for example Advanced Organic Chemistry, by Jerry March, 4^{th} Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (mCPBA), for example, in an inert solvent such as, but not limited to, dichloromethane.

### Tautomers

Compounds of the Formula I and sub-formulae thereof may exist in a number of different tautomeric forms and references to compounds of the Formula I and sub-formulae thereof include all such forms. For the avoidance of doubt, where a compound can exist in one of several tautomeric forms, and only one is specifically described or shown, all others are nevertheless embraced by Formula I and sub-formulae thereof. Examples of tautomeric forms include keto-, enol-, and enolate-forms, as in, for example, the following tautomeric pairs: keto/enol (illustrated below), pyrimidone/hydroxypyrimidine, imine/enamine, amide/imino alcohol, amidine/amidine, nitroso/oxime, thioketone/enethiol, and nitro/aci-nitro.

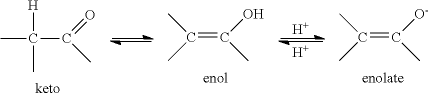

### Isomers

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric centre, for example, it is bonded to four different groups, a pair of enantiomers is possible. An enantiomer can be characterized by the absolute configuration of its asymmetric centre and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

Certain compounds of Formula I and sub-formulae thereof may have one or more asymmetric centres and therefore can exist in a number of stereoisomeric configurations. Consequently, such compounds can be synthesized and/or isolated as mixtures of enantiomers and/or as individual (pure) enantiomers, and, in the case of two or more asymmetric centres, single diastereomers and/or mixtures of diastereomers. It should be understood that the present application includes all such enantiomers and diastereomers and mixtures thereof in all ratios.

### Isotopes

The compounds of the present invention are described herein using structural formulas that do not specifically recite the mass numbers or the isotope ratios of the constituent atoms. As such it is intended that the present application includes compounds in which the constituent atoms are present in any ratio of isotope forms. For example, carbon atoms may be present in any ratio of ¹²C, ¹³C, and ¹⁴C; hydrogen atoms may be present in any ratio of ¹H, ²H, and ³H; etc. Preferably, the constituent atoms in the compounds of the present invention are present in their naturally occurring ratios of isotope forms.

### Prodrugs and Metabolites

The compounds of Formula I and sub-formulae thereof may be administered in the form of a pro-drug which is broken down in the human or animal body to release a compound of the invention. A pro-drug may be used to alter the physical properties and/or the pharmacokinetic properties of a compound of the invention. A pro-drug can be formed when the compound of the invention contains a suitable group or substituent to which a property-modifying group can be attached. Examples of pro-drugs include in vivo cleavable ester derivatives that may be formed at a carboxy group or a hydroxy group in a compound of the Formula I and in-vivo cleavable amide derivatives that may be formed at a carboxy group or an amino group in a compound of the Formula I and sub-formulae thereof.

Accordingly, the present invention includes those compounds of the Formula I and sub-formulae thereof as defined herein when made available by organic synthesis and when made available within the human or animal body by way of cleavage of a pro-drug thereof. Accordingly, the present invention includes those compounds of the Formula I that are produced by organic synthetic means and also such compounds that are produced in the human or animal body by way of metabolism of a precursor compound, that is a compound of the Formula I and sub-formulae thereof may be a synthetically-produced compound or a metabolically-produced compound.

A suitable pharmaceutically acceptable pro-drug of a compound of the Formula I and sub-formulae thereof is one that is based on reasonable medical judgement as being suitable for administration to the human or animal body without undesirable pharmacological activities and without undue toxicity.

Various forms of pro-drug have been described, for example in the following documents:
a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191 (1991);
d) H. Bundgaard, Advanced Drug Delivery Reviews, 8, 1-38 (1992);
e) H. Bundgaard, et al., Journal of Pharmaceutical Sciences, 77, 285 (1988);
f) N. Kakeya, et al., Chem. Pharm. Bull., 32, 692 (1984);
g) T. Higuchi and V. Stella, “Pro-Drugs as Novel Delivery Systems”, A.C.S. Symposium Series, Volume 14; and
h) E. Roche (editor), “Bioreversible Carriers in Drug Design”, Pergamon Press, 1987.

A suitable pharmaceutically acceptable pro-drug of a compound of the Formula I and sub-formulae thereof that possesses a carboxy group is, for example, an in vivo cleavable ester thereof. An in vivo cleavable ester of a compound of the Formula I containing a carboxy group is, for example, a pharmaceutically acceptable ester which is cleaved in the human or animal body to produce the parent acid. Suitable pharmaceutically acceptable esters for carboxy include C₁₋₆alkyl esters such as, but not limited to, methyl, ethyl and tert-butyl, C₁₋₆alkoxymethyl esters such as, but not limited to, methoxymethyl esters, C₁₋₆alkanoyloxymethyl esters such as, but not limited to, pivaloyloxymethyl esters, 3-phthalidyl esters, C₃₋₈cycloalkylcarbonyloxy-C₁₋₆alkyl esters such as, but not limited to, cyclopentylcarbonyloxymethyl and 1-cyclohexylcarbonyloxyethyl esters, 2-oxo-1,3-dioxolenylmethyl esters such as, but not limited to, 5-methyl-2-oxo-1,3-dioxolen-4-ylmethyl esters and C₁₋₆alkoxycarbonyloxy-C₁₋₆alkyl esters such as, but not limited to, methoxycarbonyloxymethyl and 1-methoxycarbonyloxyethyl esters.

A suitable pharmaceutically acceptable pro-drug of a compound of the Formula I and sub-formulae thereof that possesses a hydroxy group is, for example, an in vivo cleavable ester or ether thereof. An in vivo cleavable ester or ether of a compound of the Formula I and sub-formulae thereof containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as, but not limited to, phosphate esters (including phosphoramidic cyclic esters). Further suitable pharmaceutically acceptable ester forming groups for a hydroxy group include C₁₋₁₀alkanoyl groups such as, but not limited to, acetyl, benzoyl, phenylacetyl and substituted benzoyl and phenylacetyl groups, C₁₋₁₀alkoxycarbonyl groups such as, but not limited to, ethoxycarbonyl, N,N-(C₁₋₆)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁₋₄alkyl)piperazin-1-ylmethyl. Suitable pharmaceutically acceptable ether forming groups for a hydroxy group include a-acyloxyalkyl groups such as, but not limited to, acetoxymethyl and pivaloyloxymethyl groups.

A suitable pharmaceutically acceptable pro-drug of a compound of the Formula I and sub-formulae thereof that possesses a carboxy group is, for example, an in vivo cleavable amide thereof, for example an amide formed with an amine such as, but not limited to, ammonia, a C₁₋₄alkylamine such as, but not limited to, methylamine, a (C₁₋₄alkyl)₂amine such as, but not limited to, dimethylamine, N-ethyl-N-methylamine or diethylamine, a C₁₋₄alkoxy-C₂₋₄alkylamine such as, but not limited to, 2-methoxyethylamine, a phenyl-C₁₋₄ alkylamine such as, but not limited to, benzylamine and amino acids such as, but not limited to, glycine or an ester thereof.

A suitable pharmaceutically acceptable pro-drug of a compound of the Formula I and sub-formulae thereof that possesses an amino group is, for example, an in vivo cleavable amide derivative thereof. Suitable pharmaceutically acceptable amides from an amino group include, for example an amide formed with C₁₋₁₀alkanoyl groups such as, but not limited to, an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁₋₄alkyl)piperazin-1-ylmethyl.

The in vivo effects of a compound of the Formula I and sub-formulae thereof may be exerted in part by one or more metabolites that are formed within the human or animal body after administration of a compound of the Formula I and sub-formulae thereof. As stated herein, the in vivo effects of a compound of the Formula I and sub-formulae thereof may also be exerted by way of metabolism of a precursor compound (a pro-drug).

### Pharmaceutical Compositions

According to a further aspect of the invention there is provided a pharmaceutical composition which comprises a compound of the invention as defined herein, or a pharmaceutically acceptable salt, hydrate or solvate thereof, in association with a pharmaceutically acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

An effective amount of a compound of the present invention for use in therapy is an amount sufficient to treat or prevent a proliferative condition referred to herein, slow its progression and/or reduce the symptoms associated with the condition.

The amount of active ingredient that is combined with one or more excipients to produce a single dosage form will necessarily vary depending upon the individual treated and the particular route of administration. For example, a formulation intended for oral administration to humans will generally contain, for example, from 0.5 mg to 1.5 g of active agent (more suitably from 0.5 to 600 mg, for example from 1 to 200 mg) compounded with an appropriate and convenient amount of excipients which may vary from about 5 to about 98 percent by weight of the total composition.

The size of the dose for therapeutic or prophylactic purposes of a compound of the Formula I will naturally vary according to the nature and severity of the conditions, the age and sex of the animal or patient and the route of administration, according to well-known principles of medicine.

It is to be noted that dosages and dosing regimens may vary with the type and severity of the condition to be alleviated, and may include the administration of single or multiple doses, i.e. QD (once daily), BID (twice daily), etc., over a particular period of time (days or hours). It is to be further understood that for any particular subject or patient, specific dosage regimens may need to be adjusted over time according to the individual need and the professional judgment of the person administering or supervising the administration of the pharmaceutical compositions. For example, doses may be adjusted based on pharmacokinetic or pharmacodynamic parameters, which may include clinical effects such as toxic effects and/or laboratory values. Thus, the present application encompasses intra-patient dose-escalation as determined by the person skilled in the art. Procedures and processes for determining the appropriate dosage(s) and dosing regimen(s) are well-known in the relevant art and would readily be ascertained by the skilled artisan. As such, one of ordinary skill would readily appreciate and recognize that the dosage ranges set forth herein are exemplary only and are not intended to limit the scope or practice of the pharmaceutical compositions described herein.

In using a compound of the invention for therapeutic or prophylactic purposes it will generally be administered so that a daily dose in the range, for example, 0.1 mg/kg to 75 mg/kg body weight is received, given if required in divided doses. In general lower doses will be administered when a parenteral route is employed. Thus, for example, for intravenous or intraperitoneal administration, a dose in the range, for example, 0.1 mg/kg to 30 mg/kg body weight will generally be used. Similarly, for administration by inhalation, a dose in the range, for example, 0.05 mg/kg to 25 mg/kg body weight will be used.

For the compounds of the present invention, oral administration is particularly suitable. The compounds of the present invention may be formulated as a tablet, capsule or solution for oral administration. Suitably, the compound of the present invention is formulated in a unit dosage form (e.g. a tablet or capsule) for oral administration. Typically, unit dosage forms will contain about 0.5 mg to 1.5 g of a compound of this invention.

### Synthesis

The compounds of the present invention can be prepared by any suitable technique known in the art.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilised.

It will be appreciated that during the synthesis of the compounds of the invention in the processes defined herein, or during the synthesis of certain starting materials, it may be desirable to protect certain substituent groups to prevent their undesired reaction. The skilled chemist will appreciate when such protection is required, and how such protecting groups may be put in place, and later removed.

For Examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule.

Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as, but not limited to, acetyl, an alkoxycarbonyl group, for example a methoxycarbonyl, ethoxycarbonyl or tbutoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as, but not limited to, an alkali metal hydroxide, for example lithium or sodium hydroxide. Alternatively an acyl group such as a tertbutoxycarbonyl group may be removed, for example, by treatment with a suitable acid, such as hydrochloric, sulfuric or phosphoric acid or trifluoroacetic acid and an arylmethoxycarbonyl group such as a benzyloxycarbonyl group may be removed, for example, by hydrogenation over a catalyst such as palladium on carbon, or by treatment with a Lewis acid, for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively, an arylmethyl group such as a benzyl group may be removed, for example, by hydrogenation over a catalyst such as palladium on carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium on carbon.

Resins may also be used as a protecting group.

Once a compound of formula (1) has been synthesised by any one of the processes defined herein, the processes may then further comprise one or more of the additional steps of:
(i) removing any residual protecting groups present;
(ii) converting the compound formula (1) into another compound of formula (1);
(iii) forming a pharmaceutically acceptable salt, hydrate or solvate of the compound of formula I; and/or
(iv) forming a prodrug of the compound of formula I.

### Therapeutic Uses and Applications

The compounds of the present invention are potent inhibitors of Casein Kinase 2 alpha (CK2α). Data showing the CK2α inhibition for the exemplified compounds is presented in the accompanying example section.

The compounds of the present invention are designed to bind to the catalytic ATP site of CK2α (to drive potent enzyme inhibition) and the αD site (to drive high levels of selectivity over other kinases) [Brear et al, Chem Sci 2016].

Accordingly, the compounds of formula I are useful for the treatment and/or prevention of diseases and conditions in which CK2α activity is implicated, such as, for example, but not limited to, the treatment and/or prevention of proliferative disorders (e.g. cancer), viral infections, inflammation, diabetes, vascular and ischemic disorders, neurodegeneration and the regulation of circadian rhythm.

In another aspect, the present invention provides a compound of Formula I as defined herein, or a pharmaceutically acceptable salt, hydrate or solvate thereof, or a pharmaceutical composition as defined herein, for use in therapy.

In another aspect, the present invention provides a compound of Formula I as defined herein, or a pharmaceutically acceptable salt, hydrate or solvate thereof, or a pharmaceutical composition as defined herein, for use in the treatment of a disease or condition in which CK2α activity is implicated.

In another aspect, the present invention provides the use of a compound of Formula I as defined herein, or a pharmaceutically acceptable salt, hydrate or solvate thereof, in the manufacture of a medicament for use in the treatment of a disease or condition in which CK2α activity is implicated.

In another aspect, the present invention provides a method of treating a disease or condition in which CK2α activity is implicated, said method comprising administering to a subject in need thereof an effective amount of a compound of Formula I as defined herein, or a pharmaceutically acceptable salt, hydrate or solvate thereof, or a pharmaceutical composition as defined herein.

In another aspect, the present invention provides a compound of Formula I as defined herein, or a pharmaceutically acceptable salt, hydrate or solvate thereof, or a pharmaceutical composition as defined herein, for use in the treatment of a disease or condition associated with aberrant activity of CK2α.

In another aspect, the present invention provides the use of a compound of Formula I as defined herein, or a pharmaceutically acceptable salt, hydrate or solvate thereof, in the manufacture of a medicament for use in the treatment of a disease or condition associated with aberrant activity of CK2α.

In another aspect, the present invention provides a method of treating a disease or condition associated with aberrant activity of CK2α, said method comprising administering to a subject in need thereof an effective amount of a compound of Formula I as defined herein, or a pharmaceutically acceptable salt, hydrate or solvate thereof, or a pharmaceutical composition as defined herein.

In another aspect, the present invention provides a compound of Formula I as defined herein, or a pharmaceutically acceptable salt, hydrate or solvate thereof, or a pharmaceutical composition as defined herein, for use in the treatment of proliferative disorders (e.g. cancer or benign neoplasms), viral infections, an inflammatory disease or condition, diabetes, vascular and ischemic disorders, neurodegenerative disorders and/or the regulation of circadian rhythm.

In another aspect, the present invention provides the use of a compound of Formula I as defined herein, or a pharmaceutically acceptable salt, hydrate or solvate thereof, in the manufacture of a medicament for use in the treatment of proliferative disorders (e.g. cancer or benign neoplasms), viral infections, an inflammatory disease or condition, diabetes, vascular and ischemic disorders, neurodegenerative disorders and/or the regulation of circadian rhythm.

In another aspect, the present invention provides a method of treating a proliferative disorder (e.g. cancer or benign neoplasms), a viral infection, an inflammatory disease or condition, diabetes, vascular and ischemic disorders, neurodegenerative disorders and/or regulating cardiac rhythm, said method comprising administering to a subject in need thereof an effective amount of a compound of Formula I as defined herein, or a pharmaceutically acceptable salt, hydrate or solvate thereof, or a pharmaceutical composition as defined herein.

In another aspect, the present invention provides a compound of Formula I as defined herein, or a pharmaceutically acceptable salt, hydrate or solvate thereof, or a pharmaceutical composition as defined herein, for use in the treatment of a proliferative disorder.

In another aspect, the present invention provides the use of a compound of Formula I as defined herein, or a pharmaceutically acceptable salt, hydrate or solvate thereof, in the manufacture of a medicament for use in the treatment of a proliferative disorder (e.g. cancer or a benign neoplasms).

In another aspect, the present invention provides a method of treating a proliferative disorder (e.g. cancer or benign neoplasms), said method comprising administering to a subject in need thereof an effective amount of a compound of Formula I as defined herein, or a pharmaceutically acceptable salt, hydrate or solvate thereof, or a pharmaceutical composition as defined herein.

The terms “proliferative disorder” and “proliferative condition” are used interchangeably herein and pertain to an unwanted or uncontrolled cellular proliferation of excessive or abnormal cells which is undesired, such as, neoplastic or hyperplastic growth, whether in vitro or in vivo.

Examples of proliferative conditions include, but are not limited to, pre-malignant and malignant cellular proliferation, including but not limited to, cancers, psoriasis, bone diseases, fibroproliferative disorders (e.g. of connective tissues), and atherosclerosis.

Any type of cell may be treated, including but not limited to, lung, colon, breast, ovarian, prostate, liver, pancreas, brain, blood and skin.

In certain aspects of the present invention, the proliferative disorder is cancer, suitably a cancer selected from lung, colon/colorectal, breast, ovarian, prostate, liver, pancreas, brain, blood, cholangiocarcinoma and skin cancer.

In a particular aspect of the invention, the proliferative disorder is colon/colorectal, cholangiocarcinoma, ovarian or prostate cancer.

In a particular aspect of the invention, the proliferative disorder is colorectal cancer.

In certain aspects of the present invention, the proliferative disorder is hematopoietic tumour, including: myelogenous and granulocytic leukemia (malignancy of the myeloid and granulocytic white blood cell series); lymphatic, lymphocytic, and lymphoblastic leukemia (malignancy of the lymphoid and lymphocytic blood cell series); polycythemia vera and erythremia (malignancy of various blood cell products, but with red cells predominating);
and myelofibrosis.

A benign neoplasm may be, for example, hemangiomas, hepatocellular adenoma, cavernous haemangioma, focal nodular hyperplasia, acoustic neuromas, neurofibroma, bile duct adenoma, bile duct cystanoma, fibroma, lipomas, leiomyomas, mesotheliomas, teratomas, myxomas, nodular regenerative hyperplasia, trachomas, pyogenic granulomas, moles, uterine fibroids, thyroid adenomas, adrenocortical adenomas or pituitary adenomas. The benign neoplasm may be endometrial implants or a keratocystic odontogenic tumor.

In another aspect, the present invention provides a compound of Formula I as defined herein, or a pharmaceutically acceptable salt, hydrate or solvate thereof, or a pharmaceutical composition as defined herein, for use in the treatment of a cancer.

In another aspect, the present invention the use of a compound of Formula I as defined herein, or a pharmaceutically acceptable salt, hydrate or solvate thereof, in the manufacture of a medicament for use in the treatment of a cancer.

In another aspect, the present invention provides a method of treating cancer, said method comprising administering to a subject in need thereof an effective amount of a compound of Formula I as defined herein, or a pharmaceutically acceptable salt, hydrate or solvate thereof, or a pharmaceutical composition as defined herein.

The cancer may be non-metastatic or metastatic and which may be a solid tumour or a haematological (“liquid”) cancer. The cancer may, for example, be selected from:
(1) Carcinoma, including for example tumours derived from stratified squamous epithelia (squamous cell carcinomas) and tumours arising within organs or glands (adenocarcinomas). Examples include breast, colon, lung, prostate, ovary, esophageal carcinoma (including, but not limited to, esophageal adenocarcinoma and squamous cell carcinoma), basal-like breast carcinoma, basal cell carcinoma (a form of skin cancer), squamous cell carcinoma (various tissues), head and neck carcinoma (including, but not limited to, squamous cell carcinomas), stomach carcinoma (including, but not limited to, stomach adenocarcinoma, gastrointestinal stromal tumor), signet ring cell carcinoma, bladder carcinoma (including transitional cell carcinoma (a malignant neoplasm of the bladder)), bronchogenic carcinoma, colorectal carcinoma (including, but not limited to, colon carcinoma and rectal carcinoma), anal carcinoma, gastric carcinoma, lung carcinoma (including but not limited to small cell carcinoma (SCLC) and non-small cell carcinoma of the lung (NSCLC), lung adenocarcinoma, squamous cell carcinoma, large cell carcinoma, bronchioloalveolar carcinoma, and mesothelioma), neuroendocrine tumors (including but not limited to carcinoids of the gastrointestinal tract, breast, and other organs), adrenocortical carcinoma, thyroid carcinoma, pancreatic carcinoma (including, but not limited to, pancreatic ductal adenocarcinoma, pancreatic adenocarcinoma, acinar cell carcinoma, intraductal papillary mucinous neoplasm with invasive carcinoma, mucinous cystic neoplasm with invasive carcinoma, islet cell carcinoma and neuroendocrine tumors), breast carcinoma (including, but not limited to, ductal carcinoma, lobular carcinoma, inflammatory breast cancer, clear cell carcinoma, mucinous carcinoma), ovarian carcinoma (including, but not limited to, ovarian epithelial carcinoma or surface epithelial-stromal tumor including serous tumor, endometrioid tumor and mucinous cystadenocarcinoma, sex-cord-stromal tumor), liver and bile duct carcinoma (including, but not limited to, hepatocellular carcinoma, cholangiocarcinoma and hemangioma), prostate carcinoma, adenocarcinoma, brain tumours (including, but not limited to glioma, glioblastoma and medulloblastoma), germ cell tumors, sweat gland carcinoma, sebaceous gland carcinoma, papillary carcinoma, papillary adenocarcinoma, cystadenocarcinoma, kidney carcinoma (including, but not limited to, renal cell carcinoma, clear cell carcinoma and Wilm's tumor), medullary carcinoma, ductal carcinoma in situ or bile duct carcinoma, choriocarcinoma, seminoma, embryonal carcinoma, cervical carcinoma, uterine carcinoma (including, but not limited to, endometrial adenocarcinoma, uterine papillary serous carcinoma, uterine clear-cell carcinoma, uterine sarcomas and leiomyosarcomas, mixed mullerian tumors), testicular carcinoma, osteogenic carcinoma, epithelial carcinoma, sarcomatoid carcinoma, nasopharyngeal carcinoma, laryngeal carcinoma; oral and oropharyngeal squamous carcinoma;
(2) Sarcomas, including: osteosarcoma and osteogenic sarcoma (bone); chondrosarcoma (cartilage); leiomyosarcoma (smooth muscle); rhabdomyosarcoma (skeletal muscle); mesothelial sarcoma and mesothelioma (membranous lining of body cavities); fibrosarcoma (fibrous tissue); angiosarcoma and hemangioendothelioma (blood vessels); liposarcoma (adipose tissue); glioma and astrocytoma (neurogenic connective tissue found in the brain); myxosarcoma (primitive embryonic connective tissue); chordoma, endotheliosarcoma, lymphangiosarcoma, lymphangioendotheliosarcoma, synovioma, Ewing's sarcoma, mesenchymous and mixed mesodermal tumor (mixed connective tissue types) and other soft tissue sarcomas;
(3) Myeloma and multiple myeloma;
(4) Hematopoietic tumours, including: myelogenous and granulocytic leukemia (malignancy of the myeloid and granulocytic white blood cell series); lymphatic, lymphocytic, and lymphoblastic leukemia (malignancy of the lymphoid and lymphocytic blood cell series); polycythemia vera and erythremia (malignancy of various blood cell products, but with red cells predominating); myelofibrosis.
(5) Lymphomas, including: Hodgkin and Non-Hodgkin lymphomas;
(6) Solid tumors of the nervous system including medulloblastoma, craniopharyngioma, ependymoma, pinealoma, hemangioblastoma, acoustic neuroma, oligodendroglioma, meningioma, neuroblastoma and schwannoma;
(7) Melanoma, uveal melanoma and retinoblastoma; and
(8) Mixed Types, including, e.g., adenosquamous carcinoma, mixed mesodermal tumor, carcinosarcoma or teratocarcinoma.

Suitably, a compound of the invention, or a pharmaceutically acceptable salt thereof may be for use in the treatment of a cancer selected from cancer selected from colon/colorectal cancer, cholangiocarcinoma, gastric cancer, skin cancer (e.g. basal cell carcinoma), ovarian, prostate, breast cancer, liver cancer, pancreatic cancer, brain cancer, blood cancers (leukaemia's, myelomas), bladder cancer, bone cancer, head and neck cancer, renal cancer and lung cancer.

More suitably, the cancer is selected from colon/colorectal cancer, prostate cancer, ovarian cancer, basal cell carcinoma or cholangiocarcinoma.

In a particular aspect of the present invention, the cancer is basal cell carcinoma.

In a particular aspect of the present invention, the cancer is colorectal cancer.

In a particular aspect of the present invention, the cancer is cholangiocarcinoma.

In a further aspect of the present invention, the cancer is prostate cancer.

In a further aspect of the present invention, the cancer is ovarian cancer.

In another aspect of the present invention, the cancer is a hematopoietic tumour.

It is hypothesised that the compounds of the present invention will be particularly suited to the treatment of wnt pathway driven cancers, e.g. wnt pathway mutated colorectal cancer or cholangiocarcinoma (Di Maira et al, 2019).

In addition to CK2α having a very well characterized function in wnt pathway activity, it also plays a role in other key cellular pathways known to be upregulated in cancer, such as, but not limited to, the DNA damage response (Ruzzene & Pinna, 2010; Montenarh, Transl. Cancer Res 2016). Thus, the compounds of the present invention may have a further use in the treatment of PARP insensitive tumors in prostate/ovarian cancer.

CK2α has also recently been identified as a key host protein required for viral replication (e.g. in SARS-CoV2) and as such could represent an antiviral treatment (Gordon et al. Nature 2020).

Thus, in another aspect, the present invention provides a compound of Formula I as defined herein, or a pharmaceutically acceptable salt, hydrate or solvate thereof, or a pharmaceutical composition as defined herein, for use in the treatment of a viral infection.

In another aspect, the present invention provides the use of a compound of Formula I as defined herein, or a pharmaceutically acceptable salt, hydrate or solvate thereof, in the manufacture of a medicament for use in the treatment of a viral infection.

In another aspect, the present invention provides a method of treating a viral infection, said method comprising administering to a subject in need thereof an effective amount of a compound of Formula I as defined herein, or a pharmaceutically acceptable salt, hydrate or solvate thereof, or a pharmaceutical composition as defined herein.

Suitably, the virus is a coronavirus, e.g. SARS-CoV2.

### Routes of Administration

The compounds of the invention or pharmaceutical compositions comprising these compounds may be administered to a subject by any convenient route of administration, whether systemically/peripherally or topically (i.e., at the site of desired action).

Routes of administration include, but are not limited to, oral (e.g. by ingestion); buccal; sublingual; transdermal (e.g. by a patch, plaster, etc.); transmucosal (e.g. by a patch, plaster, etc.); intranasal (e.g. by nasal spray); ocular (e.g. by eye drops, eye ointment etc.); pulmonary (e.g. by inhalation or insufflation therapy, for example via an aerosol, for example by the nose or mouth); rectal (e.g. by suppository or enema); vaginal (e.g. by pessary); parental, for example by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir dosage form, for example subcutaneously or intramuscularly.

The compounds of the present invention are particularly suitable for oral administration.

### Combination Therapies

The compounds of the invention and salts, solvates thereof defined herein may be applied as a sole therapy or may involve, in addition to the compound of the invention, one or more additional therapeutic agents, e.g. an anti-tumour agent.

In the context of cancer treatment, in addition to the compound of the invention therapy may involve conventional surgery or radiotherapy or chemotherapy. Such chemotherapy may include one or more of the following categories of anti-tumour agents:
other antiproliferative/antineoplastic drugs and combinations thereof, as used in medical oncology, such as, but not limited to, alkylating agents (for example cisplatin, oxaliplatin, carboplatin, cyclophosphamide, nitrogen mustard, melphalan, chlorambucil, busulphan, temozolamide and nitrosoureas); antimetabolites (for example gemcitabine and antifolates such as, but not limited to, fluoropyrimidines like 5-fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);
cytostatic agents such as, but not limited to, antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5a-reductase such as, but not limited to, finasteride;
anti-invasion agents [for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341), N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661) and bosutinib (SKI-606), and metalloproteinase inhibitors like marimastat, inhibitors of urokinase plasminogen activator receptor function or antibodies to Heparanase];
inhibitors of growth factor function: for example such inhibitors include growth factor antibodies and growth factor receptor antibodies (for example the anti-erbB2 antibody trastuzumab [Herceptin™], the anti-EGFR antibody panitumumab, the anti-erbB1 antibody cetuximab [Erbitux, C225] and any growth factor or growth factor receptor antibodies disclosed by Stern et al. (Critical reviews in oncology/haematology, 2005, Vol. 54, pp 11-29); such inhibitors also include tyrosine kinase inhibitors, for example inhibitors of the epidermal growth factor family (for example EGFR family tyrosine kinase inhibitors such as, but not limited to, N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)-quinazolin-4-amine (CI 1033), erbB2 tyrosine kinase inhibitors such as, but not limited to, lapatinib); inhibitors of the hepatocyte growth factor family; inhibitors of the insulin growth factor family; inhibitors of the platelet-derived growth factor family such as, but not limited to, imatinib and/or nilotinib (AMN107); inhibitors of serine/threonine kinases (for example Ras/Raf signalling inhibitors such as, but not limited to, farnesyl transferase inhibitors, for example sorafenib (BAY 43-9006), tipifarnib (R115777) and lonafarnib (SCH66336)), inhibitors of cell signalling through MEK and/or AKT kinases, c-kit inhibitors, abl kinase inhibitors, PI3 kinase inhibitors, Plt3 kinase inhibitors, CSF-1R kinase inhibitors, IGF receptor (insulin-like growth factor) kinase inhibitors; aurora kinase inhibitors (for example AZD1152, PH739358, VX-680, MLN8054, R763, MP235, MP529, VX-528 AND AX39459) and cyclin dependent kinase inhibitors such as, but not limited to, CDK2 and/or CDK4 inhibitors;
antiangiogenic agents such as, but not limited to, those which inhibit the effects of vascular endothelial growth factor, [for example the anti-vascular endothelial cell growth factor antibody bevacizumab (Avastin™) and for example, a VEGF receptor tyrosine kinase inhibitor such as, but not limited to, vandetanib (ZD6474), vatalanib (PTK787), sunitinib (SU11248), axitinib (AG-013736), pazopanib (GW 786034) and 4-(4-fluoro-2-methylindol-5-yloxy)-6-methoxy-7-(3-pyrrolidin-1-ylpropoxy)quinazoline (AZD2171; Example 240 within WO 00/47212), compounds such as, but not limited to, those disclosed in International Patent Applications WO97/22596, WO 97/30035, WO 97/32856 and WO 98/13354 and compounds that work by other mechanisms (for example linomide, inhibitors of integrin av33 function and angiostatin)];
vascular damaging agents such as, but not limited to, Combretastatin A4 and compounds disclosed in International Patent Applications WO 99/02166, WO 00/40529, WO 00/41669, WO 01/92224, WO 02/04434 and WO 02/08213;
an endothelin receptor antagonist, for example zibotentan (ZD4054) or atrasentan;
antisense therapies, for example those which are directed to the targets listed above, such as, but not limited to, ISIS 2503, an anti-ras antisense;
gene therapy approaches, including for example approaches to replace aberrant genes such as, but not limited to, aberrant p53 or aberrant BRCA1 or BRCA2, GDEPT (gene-directed enzyme pro-drug therapy) approaches such as, but not limited to, those using cytosine deaminase, thymidine kinase or a bacterial nitroreductase enzyme and approaches to increase patient tolerance to chemotherapy or radiotherapy such as multi-drug resistance gene therapy; and
immunotherapy approaches, including for example ex-vivo and in-vivo approaches to increase the immunogenicity of patient tumour cells, such as, but not limited to, transfection with cytokines such as interleukin 2, interleukin 4 or granulocyte-macrophage colony stimulating factor, approaches to decrease T-cell anergy, approaches using transfected immune cells such as, but not limited to, cytokine-transfected dendritic cells, approaches using cytokine-transfected tumour cell lines and approaches using anti-idiotypic antibodies.

In a particular embodiment, the antiproliferative treatment defined herein may involve, in addition to the compound of the invention, conventional surgery or radiotherapy or chemotherapy.

In a further particular embodiment, the antiproliferative treatment defined herein may involve, in addition to the compound of the invention, standard chemotherapy for the cancer concerned.

In a particular embodiment, the antiproliferative treatment defined herein may involve, in addition to the compound of the invention, therapy with K-ras inhibitors and/or DNA damage repair inhibitors (e.g. PARP inhibitors).

Such conjoint treatment may be achieved by way of the simultaneous, sequential or separate dosing of the individual components of the treatment. Such combination products employ the compounds of this invention within the dosage range described herein and the other pharmaceutically-active agent within its approved dosage range.

According to this aspect of the invention there is provided a combination for use in the treatment of a cancer (for example a cancer involving a solid tumour) comprising a compound of the invention as defined herein, or a pharmaceutically acceptable salt, hydrate or solvate thereof, and another anti-tumour agent.

According to this aspect of the invention there is provided a combination for use in the treatment of a proliferative condition, such as, but not limited to, cancer (for example a cancer involving a solid tumour), comprising a compound of the invention as defined herein, or a pharmaceutically acceptable salt, hydrate or solvate thereof, and any one of the anti-tumour agents listed herein above.

In a further aspect of the invention there is provided a compound of the invention or a pharmaceutically acceptable salt, hydrate or solvate thereof, for use in the treatment of cancer in combination with another anti-tumour agent, optionally selected from one listed herein above.

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination. In one embodiment, a combination refers to a combination product.

According to a further aspect of the invention there is provided a pharmaceutical composition which comprises a compound of the invention, or a pharmaceutically acceptable salt, hydrate or solvate thereof, in combination with an anti-tumour agent (optionally selected from one listed herein above), in association with a pharmaceutically acceptable diluent or carrier.

### Biological Activity

The biological assay described in the example section (Biological Assay 1) may be used to measure the pharmacological effects of the compounds of the present invention.

Although the pharmacological properties of the compounds of formula I vary with structural change, as expected, the compounds of the invention were found to be active in the assays described in Biological Assay 1. In general, the compounds of the invention demonstrate an IC₅₀ of 500 nM or less in the assay described in Biological Assay 1, with preferred compounds of the invention demonstrating an IC₅₀ of 100 nM or less and the most preferred compounds of the invention demonstrating an IC₅₀ of 30 nM or less.

### EXAMPLES

The invention will now be illustrated, but not limited, by reference to the specific embodiments described in the following examples. Compounds are named using conventional IUPAC nomenclature, or as named by the chemical supplier.

The following synthetic procedures are provided for illustration of the methods used; for a given preparation or step the precursor used may not necessarily derive from the individual batch synthesized according to the step in the description given.

### Analytical Methods (AM)

Where examples and preparations cite analytical data, the following analytical methods were used unless otherwise specified.

All LCMS spectra were obtained by using one of the below methods.

Method 1 (AM1): (5-95 A-B_1.5 min_220 & 254 nm): Instrument: Agilent 1100G1956A; Column: Kinetex@ 5 um EVO C18 30×2.1 mm×5 m; Run Time: 1.5 min; Solvents: A) 0.0375% TFA in water (v/v), B) 0.01875% TFA in acetonitrile (v/v). The gradient runs with 5% B; Gradient: 5-95% B with A, 0.8 min; hold at 95% B to 1.2 min; 5% B at 1.21 min and hold at 5% B to 1.5 min @1.5 mL/min, 50° C.

Method 2 (AM2): (5-95 A-B_1.5 min_220 & 254 nm): Instrument: Agilent 1200G6110A; Column: Kinetex@5 um EVO C18 30×2.1 mm×5 m; Run Time: 1.5 min; Solvents: A) 0.0375% TFA in water (v/v), B) 0.01875% TFA in acetonitrile (v/v). The gradient runs with 5% B; Gradient: 5-95% B with A, 0.8 min; hold at 95% B to 1.2 min; 5% B at 1.21 min and hold at 5% B to 1.5 min @1.5 mL/min, 50° C.

Method 3 (AM3): (5-95 A-B_1.55 min_220 & 254 nm): Instrument: SHIMADZU LCMS-2020; Column: Kinetex EVO C18 30×2.1 mm×5 m; Run Time: 1.55 min; Solvents: A) 0.0375% TFA in water (v/v), B) 0.01875% TFA in acetonitrile (v/v). The gradient runs with 5% B; Gradient: 5-95% B with A, 0.8 min; hold at 95% B to 1.2 min; 5% B at 1.21 min and hold at 5% B to 1.55 min @1.5 mL/min, 50° C.

Method 4 (AM4): (5-95 A-B_1.5 min_220 & 254 nm): Instrument: Agilent 1200 LC/G1956A MSD; Column: Kinetex EVO C18 30×2.1 mm×5 m; Run Time: 1.5 min; Solvents: A) 0.0375% TFA in water (v/v), B) 0.01875% TFA in acetonitrile (v/v). The gradient runs with 5% B; Gradient: 5-95% B with A, 0.8 min; hold at 95% B to 1.2 min; 5% B at 1.21 min and hold at 5% B to 1.5 min @1.5 mL/min, 50° C.

Method 5 (AM5): (0-60 A-B_1.55 min_220 & 254 nm): Instrument: SHIMADZU LCMS-2020; Column: Kinetex EVO C18 30×2.1 mm×5 m; Run Time: 1.55 min; Solvents: A) 0.0375% TFA in water (v/v), B) 0.01875% TFA in ACN (v/v). The gradient runs with 0% B; Gradient: 0-60% B with A, 0.8 min; hold at 60% B to 1.20 min; 0% B at 1.21 min and hold at 0% B to 1.55 min @1.5 mL/min, 50° C.

Method 6 (AM6): (0-60 C-D_2.20 min_220 & 254 nm): Instrument: SHIMADZU LCMS-2020; Column: Kinetex EVO C18 30×2.1 mm×5 m; Run Time: 2.20 min; Solvents: A) 0.025% NH3·H₂O in water (v/v), B) acetonitrile. The gradient runs with 0% B; Gradient: 0-60% B with A, 1.2 min; hold at 60% B to 1.6 min; 0% B at 1.61 min and hold at 0% B to 2.2 min @1.5 mL/min, 40° C.

Method 7 (AM7): (5-95 C-D_1.5 min_R_220&254_POS): Instrument: SHIMADZU LCMS-2020; Column: Kinetex EVO C18 30×2.1 mm×5 m; Run Time: 1.5 min; Solvents A) 0.025% NH₃·H₂O in water (v/v) B) Acetonitrile. The gradient runs with 5% B. Gradient: 5-95% B with A 0.8 min, hold at 95% B to 1.2 min; 5% B at 1.21 min and hold at 5% B to 1.5 min @1.5 ml/min, 40° C.

Method 8 (AM8): (10-80 C-D_2.00 min_220 & 254 nm): Instrument: Agilent 1200G6110A; Column: ACE Excel 5 C18 30×2.1 mm×5 m; Run Time: 2.00 min; Solvents: A) 0.025% NH₃·H₂O in water (v/v), B) Acetonitrile (v/v). The gradient runs with 10% B; Gradient: 10-80% B with A, 1.2 min; hold at 80% B to 1.6 min; 10% B at 1.61 min and hold at 10% B to 2.00 min @1.0 mL/min, 40° C.

Method 9 (AM9): (10-80 A-B_7 min_220 & 254 nm): Instrument: SHIMADZU LCMS-2020; Column: AB:Xtimate C18 30×2.1 mm×3 m; Run Time: 7.0 min; Solvents: A) 0.0375% TFA in water (v/v), B) 0.01875% TFA in acetonitrile (v/v). The gradient runs with 10% B; Gradient: 10-80% B with A, 6.5 min; hold at 80% B to 7 min; 10% B at 6.5 min and hold at 10% B to 7 min @1.5 mL/min, 50° C.

Method 10 (AM10): (5-95CD_4MIN_220 & 254_POS): Instrument: SHIMADZU LCMS-2020; Column: XBridge C18 2.1×50 mm×5 m; Run Time: 1.0 min; Solvents A) 0.025% NH3·H₂O in water (v/v) B) Acetonitrile. The gradient runs with 5% B. Gradient: 5-95% B with A 3.6 min @0.6 mL/min; hold at 95% B to 3.70 min; 5% B at 3.71 min and hold at 5% B to 4.00 min @1.2 ml/min, 40° C.

Method 11 (AM11): (5-95A-B_0.8 min_220&254 nm): Instrument: SHIMADZU LCMS-2020; Column: Kinetex EVO C18 30×2.1 mm×5 m; Run Time: 1 min; Solvents: A) 0.0375% TFA in water (v/v), B) 0.01875% TFA in acetonitrile (v/v). The gradient runs with 5% B; Gradient: 5-95% B with A, 0.6 min @1.5 mL/min; hold at 95% B to 0.78 min; 5% B at 0.79 min and hold at 5% B to 0.8 min @2 mL/min, 50° C.

Method 12 (AM12): (5-95 A-B_1 min_220 & 254 nm): Instrument: SHIMADZU LCMS-2020; Column: Kinetex EVO C18 30×2.1 mm×5 m; Run Time: 1 min; Solvents: A) 0.0375% TFA in water (v/v), B) 0.01875% TFA in acetonitrile (v/v). The gradient runs with 5% B; Gradient: 5-95% B with A, 0.8 min @1.5 mL/min; hold at 95% B to 0.95 min; 5% B at 0.96 min and hold at 5% B to 1.0 min @2 mL/min, 50° C.

Method 13 (AM13): (0-60 A-B_0.8 min_220 & 254 nm): Instrument: SHIMADZU LCMS-2020; Column: Kinetex EVO C18 30×2.1 mm×5 m; Run Time: 1.55 min; Solvents: A) 0.0375% TFA in water (v/v), B) 0.01875% TFA in ACN (v/v). The gradient runs with 0% B; Gradient: 0-60% B with A, 0.6 min @1.5 mL/min; hold at 60% B to 0.78 min; 0% B at 0.79 min and hold at 0% B to 0.8 min @2 mL/min, 50° C.

Method 14 (AM14): (5-95 C-D_1 min_R_220&254_POS): Instrument: SHIMADZU LCMS-2020; Column: XBridge C18 2.1×30 mm×3.5 m; Run Time: 1.0 min; Solvents A) 0.025% NH3·H₂O in water (v/v) B) Acetonitrile. The gradient runs with 5% B. Gradient: 5-95% B with A 0.8 min @1.5 mL/min; hold at 95% B to 0.94 min; 5% B at 0.95 min and hold at 5% B to 1.0 min @2 ml/min, 40° C.

Method 15 (AM15): (5-95 N_1 min_220 & 254 nm): Instrument: SHIMADZU LCMS-2020; Column: Kinetex EVO C18 30×2.1 mm×5 m; Run Time: 1 min; Solvents: A) 10 mM NH4·HCO3 in water, B) Acetonitrile. The gradient runs with 5% B; Gradient: 5-95% B with A, 0.8 min @1.5 mL/min; hold at 95% B to 0.95 min; 5% B at 0.96 min and hold at 5% B to 1.0 min @2 mL/min, 40° C.

Method 16 (AM16): (5-95 C-D_1.2 min_220 & 254 nm): Instrument: SHIMADZU LCMS-2020; Column: XBridge C18 2.1×50 mm×5 m; Run Time: 1.2 min; Solvents A) 0.025% NH₃·H₂O in water (v/v) B) Acetonitrile. The gradient runs with 5% B. Gradient: 5-95% B with A 0.8 min @1.5 mL/min, hold at 95% B to 1.10 min; 5% B at 1.11 min and hold at 5% B to 1.2 min @2 ml/min, 40° C.

Method 21 (AM21): (5-95 A-B_4 min_220 & 254 nm): Instrument: SHIMADZU LCMS-2020; Column: Kinetex EVO C18 30×2.1 mm×5 m; Run Time: 4 min; Solvents: A) 0.0375% TFA in water (v/v), B) 0.01875% TFA in acetonitrile (v/v). The gradient runs with 5% B. Gradient: 5-95% B with A 3.6 min @0.6 mL/min; hold at 95% B to 3.70 min; 5% B at 3.71 min and hold at 5% B to 4.00 min @1.5 ml/min, 50° C.

¹H NMR spectra were acquired on a Bruker Avance III spectrometer at 400 MHz using residual undeuterated solvent as reference. The spectra were processed using interpretation software ACD Spectrus processor or equivalent software.

### Purification Methods (PM)

### Chromatography

| Purification | | | |
|---|---|---|---|
| method | Column | Eluent | Eluent Ratio |
| PM1 | SiO2 | Petroleum ether | 1 |
| PM2 | SiO2 | PE:Ethyl acetate | 1:1 |
| PM3 | SiO2 | PE:Ethyl acetate | 2:1 |
| PM4 | SiO2 | PE:Ethyl acetate | 3:1 |
| PM5 | SiO2 | PE:Ethyl acetate | 4:1 |
| PM6 | SiO2 | PE:Ethyl acetate | 5:1 |
| PM7 | SiO2 | PE:Ethyl acetate | 10:1 |
| PM8 | SiO2 | PE:Ethyl acetate | 13:1 |
| PM9 | SiO2 | PE:Ethyl acetate | 15:1 |
| PM10 | SiO2 | PE:Ethyl acetate | 16:1 |
| PM11 | SiO2 | PE:Ethyl acetate | 20:1 |
| PM12 | SiO2 | PE:Ethyl acetate | 30:1 |
| PM13 | SiO2 | PE:Ethyl acetate | 40:1 |
| PM14 | SiO2 | PE:Ethyl acetate | 50:1 |
| PM15 | SiO2 | PE:Ethyl acetate | 60:1 |
| PM16 | SiO2 | PE:Ethyl acetate | 80:1 |
| PM17 | SiO2 | PE:Ethyl acetate | 100:1 |
| PM18 | SiO2 | PE:Ethyl acetate | 200:1 |
| PM19 | SiO2 | DCM:Methanol | 100:1 to 10:1 |
| PM20 | SiO2 | PE:Ethyl acetate | 8:1 to 2:1 |
| PM21 | SiO2 | PE:Ethyl acetate | 100:1 to 30:1 |
| PM22 | SiO2 | Petroleum ether:Ethyl acetate | 1:1 to 10:1 |
| | | to Ethyl acetate:Methanol | |
| PM23 | SiO2 | Petroleum ether:Ethyl acetate | 1:1 to 0:1 |
| PM24 | SiO2 | Petroleum ether:Ethyl acetate | 1:0 to 1:1 |
| PM25 | SiO2 | Petroleum ether:Ethyl acetate | 1:1 to 1:2 |
| PM26 | SiO2 | Petroleum ether:Ethyl acetate | 1:0 to 2:1 |
| PM27 | SiO2 | Petroleum ether:Ethyl acetate | 1:0 to 3:1 |
| PM28 | SiO2 | Petroleum ether:Ethyl acetate | 1:0 to 4:1 |
| PM29 | SiO2 | Petroleum ether:Ethyl acetate | 1:0 to 5:1 |
| PM30 | SiO2 | Petroleum ether:Ethyl acetate | 3:1 to 0:1 |
| PM31 | SiO2 | Petroleum ether:Ethyl acetate | 1:0 to 20:1 |
| PM32 | SiO2 | Petroleum ether:Ethyl acetate | 2:1 to 0:1 |
| PM33 | SiO2 | Petroleum ether:Ethyl acetate | 2:1 to 1:1 |
| PM34 | SiO2 | Petroleum ether:Ethyl acetate | 3:1 to 1:1 |
| PM35 | SiO2 | Petroleum ether:Ethyl acetate | 5:1 to 1:1 |
| PM36 | SiO2 | Petroleum ether:Ethyl acetate | 5:1 to 3:1 |
| PM37 | SiO2 | Petroleum ether:Ethyl acetate | 10:1 to 0:1 |
| PM38 | SiO2 | Petroleum ether:Ethyl acetate | 10:1 to 1:1 |
| PM39 | SiO2 | Petroleum ether:Ethyl acetate | 10:1 to 2:1 |
| PM40 | SiO2 | Petroleum ether:Ethyl acetate | 10:1 to 3:1 |
| PM41 | SiO2 | Petroleum ether:Ethyl acetate | 10:1 to 5:1 |
| PM42 | SiO2 | Petroleum ether:Ethyl acetate | 20:1 to 1:1 |
| PM43 | SiO2 | Petroleum ether:Ethyl acetate | 20:1 to 2:1 |
| PM44 | SiO2 | Petroleum ether:Ethyl acetate | 20:1 to 3:1 |
| PM45 | SiO2 | Petroleum ether:Ethyl acetate | 20:1 to 5:1 |
| PM46 | SiO2 | Petroleum ether:Ethyl acetate | 20:1 to 10:1 |
| PM47 | SiO2 | Petroleum ether:Ethyl acetate | 30:1 to 4:1 |
| PM48 | SiO2 | Petroleum ether:Ethyl acetate | 50:1 to 5:1 |
| PM49 | SiO2 | Petroleum ether:Ethyl acetate | 50:1 to 30:1 |
| PM50 | SiO2 | Petroleum ether:Ethyl acetate | 50:1 to 10:1 |
| PM51 | SiO2 | Petroleum ether:Ethyl acetate | 100:1 to 5:1 |
| PM52 | SiO2 | DCM:Ethyl acetate | 1:1 |
| PM53 | SiO2 | DCM:Ethyl acetate | 1:0 to 0:1 |
| PM54 | SiO2 | Petroleum ether:Ethyl acetate | 3:1 to 1:2 |
| PM55 | SiO2 | Petroleum ether:Ethyl acetate | 5:1 to 2:1 |
| PM56 | SiO2 | Petroleum ether:Ethyl acetate | 10:1 to 8:1 |
| PM57 | SiO2 | Petroleum ether:Ethyl acetate | 20:1 to 6:1 |
| PM58 | SiO2 | Petroleum ether:Ethyl acetate | 20:1 to 9:1 |
| PM59 | SiO2 | Petroleum ether:Ethyl acetate | 20:1 to 12:1 |
| PM60 | SiO2 | Petroleum ether:Ethyl acetate | 100:1 to 20:1 |

### Reverse-Phase HPLC Conditions

| Purification | | | |
|---|---|---|---|
| Method (PM) | Column | Mobile phase | Gradient |
| PM61 | Phenomenex Synergi C18 150 × | water (0.1% FA)-ACN] | 10%-90%, |
| | 25 mm × 10 mm | | 20 min |
| PM62 | Waters Xbridge 150 mm × 25 | water (10 mM NH4HCO3) - | 18%-48%, |
| | mm × 5 μm | ACN | 10 min |
| PM63 | Phenomenex Synergi C18 150 × | water (0.1% TFA)-ACN | 25%-39%, |
| | 25 mm × 10 mm | | 15 min |
| PM64 | Phenomenex Gemini-NX C18 | water (0.1% FA)-ACN | 20%-80%, |
| | 75 × 30 mm × 3 mm | | 20 min |
| PM65 | Phenomenex luna C18 150 × 25 | water (0.1% TFA)-ACN | 21%-51%, |
| | mm × 10 mm | | 10 min |
| PM66 | Phenomenex luna C18 150 × 25 | water (0.075% TFA)-ACN | 28%-58%, |
| | mm × 10 mm | | 9 min |
| PM67 | Phenomenex Synergi C18 150 × | water (0.1% TFA)-ACN | 10%-90%, |
| | 25 mm × 10 mm | | 20 min |
| PM68 | Phenomenex Synergi C18 150 × | water (0.225% FA)-ACN | 18%-48%, |
| | 25 mm × 10 mm | | 10 min |
| PM69 | Phenomenex Synergi C18 150 × | water (0.1% HCl)-ACN | 10%-90%, |
| | 25 mm × 10 mm | | 20 min |
| PM70 | SepaFlash SW040 spherical | water (0.1% TFA)-ACN | 80%-90%, |
| | C18 20-45 mm, 100 A | | 10 min |
| PM71 | Phenomenex Synergi Max-RP | water (10 mM NH4HCO3) - | 60%-90%, |
| | 250 × 50 mm × 10 mm | ACN | 35 min |
| PM72 | Phenomenex Synergi C18 150 × | water (0.05% FA) - ACN | 14%-34%, |
| | 25 mm × 10 mm | | 10 min |
| PM73 | Phenomenex Synergi C18 150 × | water (0.05% HCl)-ACN | 64%-84%, |
| | 25 mm × 10 mm | | 10 min |
| PM74 | Phenomenex luna C18 150 × 25 | water (0.075% TFA)-ACN | 50%-80%, 9 |
| | mm × 5 mm | | min |
| PM75 | Phenomenex Synergi Max-RP | water (0.1% TFA)-ACN | 30%-60%, |
| | 250 × 50 mm × 10 mm | | 25 min |
| PM76 | Phenomenex luna C18 150 × 25 | water (0.1% TFA)-ACN | 20%-40%, |
| | mm × 10 mm | | 10 min |
| PM77 | Phenomenex Synergi C18 150 × | water (0.1% TFA)-ACN | 20%-40%, |
| | 25 mm × 10 mm | | 10 min |
| PM78 | Unisil 3-100 C18 Ultra 150 × 50 | water (0.1% TFA)-ACN | 20%-40%, |
| | mm × 3 mm | | 10 min |
| PM79 | Unisil 3-100 C18 Ultra 150 × 50 | water (0.225% FA)-ACN | 20%-40%, |
| | mm × 3 mm | | 10 min |
| PM80 | Phenomenex luna C18 150 × 25 | water (0.1% TFA)-ACN | 32%-62%, |
| | mm × 10 mm | | 11 min |
| PM81 | Phenomenex Gemini-NX C18 | water (0.1% TFA)-ACN | 25%-55%, 7 |
| | 75 × 30 mm × 3 mm | | min |
| PM82 | Phenomenex luna C18 150 × 25 | water (0.1% TFA)-ACN | 25%-55%, |
| | mm × 10 mm | | 10 min |
| PM83 | Phenomenex Gemini-NX C18 | water (0.1% TFA)-ACN | 35%-45%, 7 |
| | 75 × 30 mm × 3 mm | | min |
| PM84 | Phenomenex Synergi C18 150 × | water (0.05% HCl)-ACN | 58%-78%, |
| | 25 mm × 10 mm | | 10 min |
| PM85 | Boston Green ODS 150 × | water (0.225% FA)-ACN | 50%-80%, |
| | 30 mm × 5 mm | | 10 min |
| PM86 | Waters Xbridge 150 mm × 25 | water (NH4OH v/v)-ACN | 4%-34%, 9 |
| | mm × 5 μm | | min |
| PM87 | Phenomenex Synergi C18 150 × | water (0.1% TFA)-ACN | 30%-45%, |
| | 25 mm × 10 μm | | 15 min |
| PM88 | Phenomenex Synergi C18 150 × | water (0.1% FA)-ACN | 45%-70%, |
| | 25 mm × 10 μm | | 20 min |
| PM89 | Phenomenex Synergi C18 150 × | water (0.1% HCl)-ACN | 45%-70%, |
| | 25 mm × 10 μm | | 20 min |
| PM90 | Phenomenex Synergi C18 150 × | water (0.1% TFA)-ACN | 45%-70%, |
| | 25 mm × 10 μm | | 20 min |
| PM91 | Phenomenex Synergi C18 150 × | water (0.1% NH3•H2O)- | 45%-70%, |
| | 25 mm × 10 μm | ACN | 20 min |
| PM92 | Phenomenex Synergi C18 150 × | water (0.1% TFA)-ACN | 50%-70%, |
| | 25 mm × 10 μm | | 10 min |
| PM93 | SepaFlash SW040 spherical | water (0.1% NH3•H2O)- | 50%-70%, |
| | C18 20-45 mm, 100 A | ACN | 10 min |
| PM94 | Phenomenex Synergi C18 150 × | water (0.1% TFA)-ACN | 48%-78%, |
| | 25 mm × 10 μm | | 10 min |
| PM95 | Phenomenex Synergi Max-RP | water (0.1% TFA)-ACN | 32%-62%, |
| | 250 × 50 mm × 10 mm | | 10 min |
| PM96 | Phenomenex luna C18 150 × 40 | water (0.1% TFA)-ACN | 20%-50%, |
| | mm × 15 mm | | 10 min |
| PM97 | Waters Xbridge 150 mm × 25 | water (10 mM NH4HCO3) - | 20%-50%, |
| | mm × 5 μm | ACN | 10 min |
| PM98 | Phenomenex Synergi Max-RP | water (0.1% TFA)-ACN | 37%-67%, |
| | 250 × 50 mm × 10 mm | | 10 min |
| PM99 | Waters Xbridge 150 mm × 25 | water (10 mM NH4HCO3) - | 18%-51%, |
| | mm × 5 μm | ACN | 11 min |
| PM100 | Phenomenex Synergi C18 150 × | water (0.1% TFA)-ACN | 40%-70%, |
| | 25 mm × 10 μm | | 20 min |
| PM101 | Phenomenex Synergi C18 150 × | water (0.1% TFA)-ACN | 45%-70%, |
| | 25 mm × 10 μm | | 25 min |
| PM102 | Phenomenex Synergi C18 150 × | water (0.1% NH3•H2O)- | 45%-70%, |
| | 25 mm × 10 μm | ACN | 25 min |
| PM103 | Phenomenex Synergi C18 150 × | water (0.1% TFA)-ACN | 45%-60%, |
| | 25 mm × 10 μm | | 22 min |
| PM104 | Phenomenex luna C18 150 × 40 | water (0.1% TFA)-ACN | 52%-82%, |
| | mm × 15 mm | | 10 min |
| PM105 | Phenomenex Synergi C18 150 × | water (0.1% FA)-ACN] | 40%-71%, |
| | 25 mm × 10 μm | | 20 min |
| PM106 | Phenomenex Synergi C18 150 × | water (0.1% TFA)-ACN | 30%-50%, |
| | 25 mm × 10 μm | | 20 min |
| PM107 | Waters Xbridge 150 mm × 25 | water (0.1% ammonium | 0%-20%, 9 |
| | mm × 5 μm | hydroxide)-ACN | min |
| PM108 | SepaFlash SW120 spherical | water (0.1% NH3•H2O)- | 45%-65%, |
| | C18 20-45 μm, 100 A | ACN | 15 min |
| PM109 | SepaFlash SW080 spherical | water (0.1% NH3•H2O)- | 40%-65%, |
| | C18 20-45 μm, 100 A | ACN | 15 min |
| PM110 | Phenomenex Synergi C18 150 × | water (0.1% TFA)-ACN | 50%-60%, |
| | 25 mm × 10 μm | | 25 min |
| PM111 | Phenomenex Gemini-NX C18 | water (0.1% TFA)-ACN | 48%-58%, 7 |
| | 75 × 30 mm × 3 mm | | min |
| PM112 | Phenomenex Synergi C18 150 × | water (0.1% HCl)-ACN | 20%-30%, |
| | 25 mm × 10 μm | | 15 min |
| PM113 | Phenomenex luna C18 150 × 25 | water (0.1% TFA)-ACN | 5%-35%, 10 |
| | mm × 10 mm | | min |
| PM114 | Phenomenex Gemini-NX C18 | water (0.1% TFA)-ACN | 50%-60%, 7 |
| | 75 × 30 mm × 3 mm | | min |
| PM115 | Phenomenex Synergi C18 150 × | water (0.1% HCl)-ACN | 42%-60%, |
| | 25 mm × 10 μm | | 20 min |
| PM116 | Phenomenex Synergi C18 150 × | water (0.1% HCl)-ACN | 30%-50%, |
| | 25 mm × 10 μm | | 15 min |
| PM117 | Phenomenex Synergi C18 150 × | water (0.1% HCl)-ACN | 50%-70%, |
| | 25 mm × 10 μm | | 25 min |
| PM118 | Phenomenex Synergi C18 150 × | water (0.1% HCl)-ACN | 65%-90%, |
| | 25 mm × 10 μm | | 25 min |
| PM119 | Phenomenex Synergi C18 150 × | water (0.1% TFA)-ACN | 65%-75%, |
| | 25 mm × 10 μm | | 20 min |
| PM120 | Phenomenex luna C18 150 × 40 | water (0.1% TFA)-ACN | 65%- |
| | mm × 15 mm | | 95%, 10 min |
| PM121 | Phenomenex luna C18 150 × 25 | water (0.1% TFA)-ACN | 58%- |
| | mm × 10 mm | | 78%, 10 min |
| PM122 | Phenomenex Gemini-NX C18 | water (0.1% TFA)-ACN | 35%-45%, |
| | 75 × 30 mm × 3 mm | | 7 min |
| PM123 | Phenomenex luna C18 150 × 40 | water (0.1% TFA)-ACN | 62%-92%, |
| | mm × 15 mm | | 10 min |
| PM124 | Phenomenex luna C18 150 × 40 | water (0.1% TFA)-ACN | 65%-75%, 7 |
| | mm × 15 mm | | min |
| PM125 | Phenomenex Synergi C18 150 × | water (0.1% TFA)-ACN | 62%-72%, |
| | 25 mm × 10 μm | | 10 min |
| PM126 | Phenomenex Synergi C18 150 × | water (0.1% FA)-ACN] | 50%-60% |
| | 25 mm × 10 μm | | 10 min |
| PM127 | Phenomenex Synergi C18 150 × | water (0.1% TFA)-ACN | 50%-60%, |
| | 25 mm × 10 μm | | 20 min |
| PM128 | Phenomenex Synergi C18 150 × | water (0.1% TFA)-ACN | 40%-60%, |
| | 25 mm × 10 μm | | 20 min |
| PM129 | Phenomenex Synergi C18 150 × | water (0.1% HCl)-ACN | 52%-72%, 9 |
| | 25 mm × 10 μm | | min |
| PM130 | SepaFlash SW040 spherical | water (0.1% NH3•H2O)- | 50%-65%, |
| | C18 20-45 mm, 100 A | ACN | 15 min |
| PM131 | SepaFlash SW040 spherical | water (0.1% NH3•H2O)- | 70%-85%, |
| | C18 20-45 mm, 100 A | ACN | 15 min |
| PM132 | SepaFlash SW040 spherical | water (0.1% NH3•H2O)- | 50%-70%, |
| | C18 20-45 mm, 100 A | ACN | 15 min |
| PM133 | SepaFlash SW040 spherical | water (0.1% NH3•H2O)- | 45%-60%, |
| | C18 20-45 mm, 100 A | ACN | 15 min |
| PM134 | Phenomenex Synergi C18 150 × | water (0.1% NH3•H2O)- | 45%-60%, |
| | 25 mm × 10 μm | ACN | 15 min |
| PM135 | SepaFlash SW040 spherical | water (0.1% NH3•H2O)- | 65%-80%, |
| | C18 20-45 mm, 100 A | ACN | 15 min |
| PM136 | SepaFlash SW080 spherical | water (0.1% NH3•H2O)- | 30%-50%, |
| | C18 20-45 μm, 100 A | ACN | 12 min |
| PM137 | SepaFlash SW330 spherical | water (0.1% TFA)-ACN | 25%-45%, |
| | C18 20-45 μm, 100 A | | 15 min |
| PM138 | SepaFlash SW040 spherical | water (0.1% NH3•H2O)- | 60%-75%, |
| | C18 20-45 mm, 100 A | ACN | 15 min |
| PM139 | SepaFlash SW080 spherical | water (0.1% TFA)-ACN | 70%-90%, |
| | C18 20-45 μm, 100 A | | 15 min |
| PM140 | SepaFlash SW040 spherical | water (0.1% HCl)-ACN | 35%-50%, |
| | C18 20-45 mm, 100 A | | 15 min |
| PM141 | SepaFlash SW040 spherical | water (0.1% TFA)-ACN | 60%-70%, |
| | C18 20-45 mm, 100 A | | 10 min |
| PM142 | Phenomenex Synergi C18 150 × | water (0.1% NH3•H2O)- | 40%-65%, |
| | 25 mm × 10 μm | ACN | 20 min |
| PM143 | Phenomenex Synergi C18 150 × | water (0.1% FA)-ACN] | 40%-65%, |
| | 25 mm × 10 μm | | 20 min |
| PM144 | Phenomenex Synergi C18 150 × | water (0.1% NH3•H2O)- | 35%-55%, |
| | 25 mm × 10 μm | ACN | 20 min |
| PM145 | Phenomenex Synergi C18 150 × | water (0.1% NH3•H2O)- | 65%-75%, |
| | 25 mm × 10 μm | ACN | 25 min |
| PM146 | Phenomenex luna C18 150 × 25 | water (0.1% TFA)-ACN | 46%-76%, |
| | mm × 10 mm | | 10 min |
| PM147 | Phenomenex Synergi C18 150 × | water (0.1% NH3•H2O)- | 80%-90%, |
| | 25 mm × 10 μm | ACN | 20 min |
| PM148 | Phenomenex Synergi C18 150 × | water (0.1% NH3•H2O)- | 75%-85%, |
| | 25 mm × 10 μm | ACN | 20 min |
| PM149 | Phenomenex Synergi C18 150 × | water (0.1% NH3•H2O)- | 70%-80%, |
| | 25 mm × 10 μm | ACN | 20 min |
| PM150 | Boston Green ODS 150 × | water (0.225% FA)-ACN | 51%-81%, |
| | 30 mm × 5 mm | | 10 min |
| PM151 | Phenomenex Synergi C18 150 × | water (0.1% TFA)-ACN | 60%-80%, |
| | 25 mm × 10 μm | | 10 min |
| PM152 | Phenomenex Synergi C18 150 × | water (0.1% TFA)-ACN | 45%- |
| | 25 mm × 10 μm | | 75%, 10 min |
| PM153 | Waters Xbridge 150 mm × 25 | water (0.225% FA)-ACN | 22%-52%, |
| | mm × 5 μm | | 10 min |
| PM154 | Phenomenex luna C18 150 × 25 | water (0.1% TFA)-ACN | 22%-52%, |
| | mm × 10 mm | | 10 min |
| PM155 | Phenomenex luna C18 150 × 25 | water (0.1% TFA)-ACN | 25%-65%, |
| | mm × 10 mm | | 10 min |
| PM156 | Phenomenex luna C18 150 × 25 | water (0.1% TFA)-ACN | 29%-59%, |
| | mm × 10 mm | | 10 min |
| PM157 | Phenomenex Synergi C18 150 × | water (10 mM NH4HCO3) - | 62%-92%, 8 |
| | 25 mm × 10 μm | ACN | min |
| PM158 | SepaFlash SW040 spherical | water (0.1% NH3•H2O)- | 80%-90%, |
| | C18 20-45 mm, 100 A | ACN | 10 min |
| PM159 | SepaFlash SW040 spherical | water (0.1% NH3•H2O)- | 75%-85%, |
| | C18 20-45 mm, 100 A | ACN | 10 min |
| PM160 | SepaFlash SW040 spherical | water (0.1% NH3•H2O)- | 72%-89%, |
| | C18 20-45 mm, 100 A | ACN | 15 min |
| PM161 | Phenomenex Synergi C18 150 × | water (0.1% NH3•H2O)- | 20%-50%, |
| | 25 mm × 10 μm | ACN | 20 min |
| PM162 | Phenomenex Synergi C18 150 × | water (0.1% HCl)-ACN | 50%-70%, |
| | 25 mm × 10 μm | | 20 min |
| PM163 | Phenomenex Synergi C18 150 × | water (10 mM NH4HCO3) - | 30%-40%, |
| | 25 mm × 10 μm | ACN | 20 min |
| PM164 | Phenomenex luna C18 150 × 25 | water (0.1% TFA)-ACN | 35%-65%, |
| | mm × 10 mm | | 10 min |
| PM165 | Phenomenex Synergi C18 150 × | water (0.1% HCl)-ACN | 50%-73%, |
| | 25 mm × 10 μm | | 20 min |
| PM166 | Phenomenex Synergi C18 150 × | water (0.1% HCl)-ACN | 43%-70%, |
| | 25 mm × 10 μm | | 20 min |
| PM167 | Phenomenex Synergi C18 150 × | water (0.1% TFA)-ACN | 18%-38%, |
| | 25 mm × 10 μm | | 10 min |
| PM168 | Phenomenex Synergi C18 150 × | water (0.1% TFA)-ACN | 35%-55%, |
| | 25 mm × 10 μm | | 10 min |
| PM169 | Phenomenex Synergi C18 150 × | water (0.1% TFA)-ACN | 45%-60%, |
| | 25 mm × 10 μm | | 20 min |
| PM170 | Phenomenex Synergi C18 150 × | water (0.1% FA)-ACN] | 40%-55%, |
| | 25 mm × 10 μm | | 20 min |
| PM171 | SepaFlash SW040 spherical | water (0.1% TFA)-ACN | 57%-70%, |
| | C18 20-45 mm, 100 A | | 15 min |
| PM172 | SepaFlash SW040 spherical | water (0.1% NH3•H2O)- | 82%-95%, |
| | C18 20-45 mm, 100 A | ACN | 15 min |
| PM173 | SepaFlash SW040 spherical | water (0.1% NH3•H2O)- | 60%-80%, |
| | C18 20-45 mm, 100 A | ACN | 15 min |
| PM174 | SepaFlash SW040 spherical | water (0.1% TFA)-ACN | 48%-65%, |
| | C18 20-45 mm, 100 A | | 15 min |
| PM175 | SepaFlash SW040 spherical | water (0.1% NH3•H2O)- | 60%-78%, |
| | C18 20-45 mm, 100 A | ACN | 15 min |
| PM176 | SepaFlash SW040 spherical | water (0.1% TFA)-ACN | 60%-70%, |
| | C18 20-45 mm, 100 A | | 10 min |
| PM177 | SepaFlash SW040 spherical | water (0.1% NH3•H2O)- | 35%-45%, |
| | C18 20-45 mm, 100 A | ACN | 15 min |
| PM178 | SepaFlash SW040 spherical | water (0.1% NH3•H2O)- | 60%-70%, |
| | C18 20-45 mm, 100 A | ACN | 15 min |
| PM179 | SepaFlash SW040 spherical | water (0.1% NH3•H2O)- | 75%-90%, |
| | C18 20-45 mm, 100 A | ACN | 15 min |
| PM180 | SepaFlash SW040 spherical | water (0.1% NH3•H2O)- | 55%-65%, |
| | C18 20-45 mm, 100 A | ACN | 15 min |
| PM181 | SepaFlash SW040 spherical | water (0.1% NH3•H2O)- | 80%-100%, |
| | C18 20-45 mm, 100 A | ACN | 15 min |
| PM182 | SepaFlash SW040 spherical | water (0.1% TFA)-ACN | 48%-60%, |
| | C18 20-45 mm, 100 A | | 15 min |
| PM183 | Boston pH-lex 150 mm × | water (0.1% TFA)-ACN | 92%-100%, |
| | 25 mm × 10 mm | | 10 min |
| PM184 | Phenomenex Synergi C18 150 × | water (0.1% FA)-ACN] | 55%-71%, |
| | 25 mm × 10 μm | | 20 min |
| PM185 | Phenomenex Synergi C18 150 × | water (0.1% HCl)-ACN | 20%-30%, |
| | 25 mm × 10 μm | | 10 min |
| PM186 | Phenomenex Synergi C18 150 × | water (0.1% FA)-ACN] | 50%-72%, |
| | 25 mm × 10 μm | | 20 min |
| PM187 | Phenomenex Gemini 150 mm | water (0.04% NH3•H2O + | 62%-92%, |
| | 25 mm × 10 mm | 10 mM NH4HCO3) - ACN | 10 min |
| PM188 | Phenomenex Synergi C18 150 × | water (0.1% TFA)-ACN | 36%-56%, |
| | 25 mm × 10 μm | | 10 min |
| PM189 | SepaFlash SW120 spherical | water (0.1% TFA)-ACN | 40%-60%, |
| | C18 20-45 mm, 100 A | | 15 min |
| PM190 | Phenomenex Synergi C18 150 × | water (0.1% NH3•H2O)- | 70%-78%, |
| | 25 mm × 10 μm | ACN | 22 min |
| PM191 | Phenomenex Synergi C18 150 × | water-ACN | 30%-45%, |
| | 25 mm × 10 μm | | 20 min |
| PM192 | SepaFlash SW040 spherical | water (0.1% TFA)-ACN | 45%-65%, |
| | C18 20-45 mm, 100 A | | 15 min |
| PM193 | SepaFlash SW040 spherical | water (0.1% NH3•H2O)- | 45%-65%, |
| | C18 20-45 μm, 100 A | ACN | 15 min |
| PM194 | SepaFlash SW080 spherical | water (0.1% NH3•H2O)- | 70%-85%, |
| | C18 20-45 mm, 100 A | ACN | 15 min |
| PM195 | Waters Xbridge 150 mm × 25 | water (0.1% NH3•H2O)- | 5%-35%, 9 |
| | mm × 5 μm | ACN | min |
| PM196 | SepaFlash SW040 spherical | water (0.1% NH3•H2O)- | 50%-60%, |
| | C18 20-45 μm, 100 A | ACN | 15 min |
| PM197 | SepaFlash SW040 spherical | water (0.1% TFA)-ACN | 20%-30%, |
| | C18 20-45 mm, 100 A | | 15 min |
| PM198 | SepaFlash SW040 spherical | water (0.1% FA)-ACN] | 50%-70%, |
| | C18 20-45 mm, 100 A | | 15 min |
| PM199 | SepaFlash SW040 spherical | water (0.1% TFA)-ACN | 50%-70%, |
| | C18 20-45 mm, 100 A | | 15 min |
| PM200 | Phenomenex Synergi C18 150 × | water (0.1% HCl)-ACN | 50%-70%, |
| | 25 mm × 10 μm | | 15 min |
| PM201 | SepaFlash SW220 spherical | water (0.1% NH3•H2O)- | 70%-90%, |
| | C18 20-45 mm, 100 A | ACN | 30 min |
| PM202 | SepaFlash SW040 spherical | water (0.1% TFA)-ACN | 65%-85%, |
| | C18 20-45 mm, 100 A | | 15 min |
| PM203 | SepaFlash SW040 spherical | water (0.1% NH3•H2O)- | 70%-90%, |
| | C18 20-45 mm, 100 A | ACN | 12 min |
| PM204 | SepaFlash SW040 spherical | water (0.1% NH3•H2O)- | 40%-50%, |
| | C18 20-45 mm, 100 A | ACN | 15 min |
| PM205 | Unisil 3-100 C18 Ultra 150 × 50 | water (0.225% FA)-ACN | 18%-38%, |
| | mm × 3 mm | | 10 min |
| PM206 | Phenomenex Synergi C18 150 × | water (0.05% HCl)-ACN | 14%-34%, |
| | 25 mm × 10 μm | | 10 min |
| PM207 | Phenomenex Synergi C18 150 × | water (0.1% TFA)-ACN | 25%-45%, |
| | 25 mm × 10 μm | | 10 min |
| PM208 | Phenomenex Luna C18 150 × 25 | water (0.1% TFA)-ACN | 25%-45%, |
| | mm × 10 μm | | 10 min |
| PM209 | Phenomenex Luna C18 150 × 25 | water (0.1% TFA)-ACN | 22%-52%, 9 |
| | mm × 10 μm | | min |
| PM210 | Waters Xbridge 150 mm × 25 | water (10 mM NH4HCO3) - | 22%-52%, 9 |
| | mm × 5 μm | ACN | min |
| PM211 | Phenomenex Synergi C18 150 × | water (0.05% HCl)-ACN | 21%-41%, |
| | 25 mm × 10 μm | | 10 min |
| PM212 | Phenomenex Synergi C18 150 × | water (0.05% HCl)-ACN | 17%-37%, |
| | 25 mm × 10 μm | | 10 min |
| PM213 | Phenomenex Luna C18 150 × 25 | water (0.1% TFA)-ACN | 8%-38%, 10 |
| | mm × 10 μm | | min |
| PM214 | Phenomenex Gemini-NX C18 | water (0.1% TFA)-ACN | 25%-35%, 7 |
| | 75 × 30 mm × 3 mm | | min |
| PM215 | Phenomenex Luna C18 150 × 25 | water (0.1% TFA)-ACN | 27%-47%, |
| | mm × 10 μm | | 10 min |
| PM216 | Phenomenex Luna C18 150 × 25 | water (0.05% HCl)-ACN | 15%-35%, |
| | mm × 10 μm | | 6.5 min |
| PM217 | Phenomenex Luna C18 150 × 25 | water (0.1% TFA)-ACN | 32%-52%, |
| | mm × 10 μm | | 10 min |
| PM218 | Phenomenex Luna C18 150 × 25 | water (0.075% TFA)-ACN | 15%-45%, 9 |
| | mm × 10 μm | | min |
| PM219 | Phenomenex Luna C18 150 × 25 | water (0.1% TFA)-ACN | 15%-45%, 9 |
| | mm × 10 μm | | min |
| PM220 | Phenomenex Gemini-NX C18 | water (0.1% TFA)-ACN | 22%-32%, 7 |
| | 75 × 30 mm × 3 mm | | min |
| PM221 | Phenomenex Luna C18 150 × 25 | water (0.1% TFA)-ACN | 24%-44%, 9 |
| | mm × 10 μm | | min |
| PM222 | Phenomenex Luna C18 150 × 25 | water (0.1% TFA)-ACN | 12%- |
| | mm × 10 μm | | 42%, 10 min |
| PM223 | Phenomenex Gemini-NX C18 | water (0.1% TFA)-ACN | 28%-38%, 7 |
| | 75 × 30 mm × 3 mm | | min |
| PM224 | Phenomenex Synergi C18 150 × | water (0.225% FA)-ACN | 20%-50%, 9 |
| | 25 mm × 10 μm | | min |
| PM225 | Waters Xbridge 150 mm × 25 | water (10 mM NH4HCO3) - | 20%-50%, 9 |
| | mm × 5 μm | ACN | min |
| PM226 | Phenomenex Synergi C18 150 × | water (0.1% TFA)-ACN | 12%-32%, |
| | 25 mm × 10 μm | | 10 min |
| PM227 | Unisil 3-100 C18 Ultra 150 × 50 | water (0.225% FA)-ACN | 12%-32%, |
| | mm × 3 mm | | 10 min |
| PM228 | Phenomenex Synergi C18 150 × | water (0.1% TFA)-ACN | 25%-40%, 9 |
| | 25 mm × 10 μm | | min |
| PM229 | Phenomenex Gemini-NX C18 | water (0.1% TFA)-ACN | 8%-38%, 7 |
| | 75 × 30 mm × 3 mm | | min |
| PM230 | Phenomenex Gemini-NX C18 | water (0.1% TFA)-ACN | 12%-42%, 7 |
| | 75 × 30 mm × 3 mm | | min |
| PM231 | Phenomenex Luna C18 150 × 25 | water (0.1% TFA)-ACN | 14%-44%, |
| | mm × 10 μm | | 10 min |
| PM232 | Phenomenex Luna C18 150 × 25 | water (0.1% TFA)-ACN | 15%-45%, |
| | mm × 10 μm | | 10 min |
| PM233 | Phenomenex Gemini-NX C18 | water (0.1% TFA)-ACN | 18%-28%, 7 |
| | 75 × 30 mm × 3 mm | | min |
| PM234 | Waters Xbridge 150 mm × 25 | water (10 mM NH4HCO3) - | 18%-48%, |
| | mm × 5 μm | ACN | 11 min |
| PM235 | Phenomenex Synergi C18 150 × | water (10 mM NH4HCO3) - | 22%-52%, 8 |
| | 25 mm × 10 μm | ACN | min |
| PM236 | Phenomenex Synergi C18 150 × | water (10 mM NH4HCO3) - | 23%-53%, 8 |
| | 25 mm × 10 μm | ACN | min |
| PM237 | Waters Xbridge 150 mm × 25 | water (10 mM NH4HCO3) - | 23%-53%, |
| | mm × 5 μm | ACN | 11 min |
| PM238 | SepaFlash SW040 spherical | water (0.1% NH3•H2O)- | 45%-55%, |
| | C18 20-45 mm, 100 A | ACN | 15 min |
| PM239 | Phenomenex Synergi C18 150 × | water (0.1% TFA)-ACN | 42%-71%, |
| | 25 mm × 10 μm | | 25 min |
| PM240 | Phenomenex Luna C18 150 × 25 | water (0.05% HCl)-ACN | 4%-34%, 10 |
| | mm × 10 μm | | min |
| PM241 | SepaFlash SW330 spherical | water (0.1% TFA)-ACN | 25%-40%, |
| | C18 20-45 mm, 100 A | | 15 min |
| PM242 | Waters Xbridge 150 mm × 25 | water (0.05% ammonium | 10%-37%, |
| | mm × 5 μm | hydroxide)-ACN | 10 min |
| PM243 | Phenomenex Luna C18 150 × 25 | water (0.1% TFA)-ACN | 12%-42%, |
| | mm × 10 μm | | 10 min |
| PM244 | Welch Xtimate C18 150 × | water (0.1% TFA)-ACN | 12%-42%, |
| | 25 mm × 5 mm | | 10 min |
| PM245 | Phenomenex Luna C18 150 × 25 | water (0.1% TFA)-ACN | 18%-48%, |
| | mm × 10 μm | | 11 min |
| PM246 | Welch Xtimate C18 150 × | water (0.1% TFA)-ACN | 14%-44%, |
| | 25 mm × 5 mm | | 10 min |
| PM247 | SepaFlash SW040 spherical | water (0.1% NH3•H2O)- | 35%-50%, |
| | C18 20-45 mm, 100 A | ACN | 12 min |
| PM248 | SepaFlash SW040 spherical | water (0.1% NH3•H2O)- | 40%-55%, |
| | C18 20-45 mm, 100 A | ACN | 11 min |
| PM249 | Waters Xbridge 150 mm × 25 | water (ammonium | 18%-48%, 8 |
| | mm × 5 μm | hydroxide v/v)-ACN | min |
| PM250 | Phenomenex Synergi C18 150 × | water (10 mM NH4HCO3) - | 18%-48%, 8 |
| | 25 mm × 10 μm | ACN | min |
| PM251 | Welch Ultimate C18 150 × 25 | water (0.1% TFA)-ACN | 20%-50%, 2 |
| | mm × 5 mm | | min |
| PM252 | Welch Ultimate C18 150 × 25 | water (0.1% TFA)-ACN | 20%-50%, 7 |
| | mm × 5 mm | | min |
| PM253 | Phenomenex Gemini-NX C18 | water (0.05% ammonium | 20%-50%, 7 |
| | 75 × 30 mm × 3 mm | hydroxide)-ACN | min |
| PM254 | Phenomenex Luna C18 150 × 25 | water (0.225% FA)-ACN | 3%-33%, 11 |
| | mm × 10 μm | | min |
| PM255 | Phenomenex Gemini-NX C18 | water (0.05% ammonium | 15%-45%, 7 |
| | 75 × 30 mm × 3 mm | hydroxide)-ACN | min |
| PM256 | SepaFlash SW040 spherical | water (0.1% NH3•H2O)- | 40%-57%, |
| | C18 20-45 mm, 100 A | ACN | 15 min |
| PM257 | SepaFlash SW040 spherical | water (0.1% NH3•H2O)- | 40%-55%, |
| | C18 20-45 mm, 100 A | ACN | 15 min |
| PM258 | Phenomenex Luna C18 150 × 25 | water (0.1% TFA)-ACN | 9%-39%, 10 |
| | mm × 10 μm | | min |
| PM259 | Phenomenex Luna C18 150 × 25 | water (0.1% TFA)-ACN | 16%-46%, |
| | mm × 10 μm | | 10 min |
| PM260 | Phenomenex Luna C18 150 × 25 | water (0.1% TFA)-ACN | 1%-24%, 10 |
| | mm × 10 μm | | min |
| PM261 | Phenomenex Luna C18 150 × 25 | water (0.1% TFA)-ACN | 1%-30%, 10 |
| | mm × 10 μm | | min |
| PM262 | Phenomenex Synergi Max-RP | water (0.1% TFA)-ACN | 8%-38%, 10 |
| | C18 250 × 50 mm × 10 μm | | min |
| PM263 | Phenomenex Luna C18 150 × 25 | water (0.1% TFA)-ACN | 17%-47%, |
| | mm × 10 μm | | 10 min |
| PM264 | Welch Xtimate C18 150 × | water (0.1% TFA)-ACN | 8%-38%, 10 |
| | 25 mm × 5 mm | | min |
| PM265 | Phenomenex Gemini-NX C18 | water (0.1% TFA)-ACN | 15%-25%, 7 |
| | 75 × 30 mm × 3 mm | | min |
| PM266 | Phenomenex Luna C18 150 × 25 | water (0.1% TFA)-ACN | 15%-45%, 2 |
| | mm × 10 μm | | min |
| PM267 | Waters Xbridge 150 mm × 25 | water (10 mM NH4HCO3) - | 27%-57%, 9 |
| | mm × 5 μm | ACN | min |
| PM268 | Phenomenex Gemini-NX C18 | water (10 mM NH4HCO3) - | 20%-50%, 8 |
| | 75 × 30 mm × 3 mm | ACN | min |
| PM269 | Zhongpu RD-C18 150 mm × 25 | water (0.225% FA)-ACN | 21%-51%, |
| | mm × 3 mm | | 10 min |
| PM270 | Unisil 3-100 C18 Ultra 150 × 50 | water (0.225% FA)-ACN | 25%-45%, |
| | mm × 3 mm | | 10 min |
| PM271 | Phenomenex Gemini-NX C18 | water (0.05% ammonium | 14%-44%, |
| | 75 × 30 mm × 3 mm | hydroxide)-ACN | 11.5 min |
| PM272 | Phenomenex Gemini-NX C18 | water (0.05% ammonium | 10%-40%, 8 |
| | 75 × 30 mm × 3 mm | hydroxide)-ACN | min |
| PM273 | Phenomenex Synergi C18 150 × | water (0.1% TFA)-ACN | 30%-50%, |
| | 25 mm × 10 μm | | 10 min |
| PM274 | Shim-pack C18 150 × 25 mm × | water (0.225% FA)-ACN | 17%-37%, |
| | 10 mm | | 10 min |
| PM275 | Shim-pack C18 150 × 25 mm × | water (0.225% FA)-ACN | 23%-43%, |
| | 10 mm | | 10 min |
| PM276 | Welch Ultimate XB-CN 250 × 70 | Hexane-EtOH | 35%-75%, |
| | mm × 10 mm | | 15 min |
| PM277 | Phenomenex Luna C18 150 × 25 | water (0.1% NH3•H2O)- | 35%-75%, |
| | mm × 10 μm | ACN | 15 min |
| PM278 | Phenomenex Gemini-NX C18 | water (0.05% ammonium | 33%-63%, |
| | 75 × 30 mm × 3 mm | hydroxide)-ACN | 11.5 min |
| PM279 | Welch Ultimate XB-CN 250 × 70 | water (0.1% NH3•H2O)- | 30%-70%, |
| | mm × 10 mm | ACN | 15 min |
| PM280 | Phenomenex Gemini-NX C18 | water (0.05% ammonium | 30%-60%, 7 |
| | 75 × 30 mm × 3 mm | hydroxide)-ACN | min |
| PM281 | Phenomenex Luna C18 150 × 25 | water (0.1% TFA)-ACN | 30%-60%, 7 |
| | mm × 10 μm | | min |
| PM282 | Phenomenex Gemini-NX C18 | water (ammonium | 28%-58%, 7 |
| | 75 × 30 mm × 3 mm | hydroxide v/v)-ACN | min |
| PM283 | Phenomenex Luna C18 150 × 25 | water (0.225% FA)-ACN | 15%-45%, |
| | mm × 10 μm | | 11 min |
| PM284 | Welch Ultimate XB-CN 250 × 70 | water (0.1% NH3•H2O)- | 15%-55%, |
| | mm × 10 mm | ACN | 15 min |
| PM285 | Waters Xbridge 150 mm × 25 | water (ammonium | 12%-42%, |
| | mm × 5 μm | hydroxide v/v)-ACN | 11 min |
| PM286 | Phenomenex Gemini-NX C18 | water (ammonium | 8%-38%, |
| | 75 × 30 mm × 3 mm | hydroxide v/v)-ACN | 11.5 min |
| PM287 | Phenomenex Gemini-NX C18 | water (0.05% ammonium | 25%-55%, |
| | 75 × 30 mm × 3 mm | hydroxide)-ACN | 11.5 min |
| PM288 | Phenomenex Gemini-NX C18 | water (0.05% ammonium | 29%-59%, 7 |
| | 75 × 30 mm × 3 mm | hydroxide)-ACN | min |
| PM289 | Unisil 3-100 C18 Ultra 150 × 50 | water (0.225% FA)-ACN | 22%-42%, |
| | mm × 3 mm | | 10 min |
| PM290 | Shim-pack C18 150 × 25 mm × | water (0.225% FA)-ACN | 22%-42%, |
| | 10 mm | | 10 min |
| PM291 | Shim-pack C18 150 × 25 mm × | water (0.225% FA)-ACN | 24%-46%, |
| | 10 mm | | 11 min |
| PM292 | Phenomenex Gemini-NX C18 | water (0.05% ammonium | 9%-39%, 8 |
| | 75 × 30 mm × 3 mm | hydroxide)-ACN | min |
| PM293 | Phenomenex Gemini-NX C18 | water (10 mM NH4HCO3) - | 17%-47%, 8 |
| | 75 × 30 mm × 3 mm | ACN | min |
| PM294 | Welch Ultimate XB-CN 250 × 70 | Hexane-EtOH | 20%-60%, |
| | mm × 10 mm | | 15 min |
| PM295 | Phenomenex Synergi C18 150 × | water (0.1% TFA)-ACN | 15%-45%, 6 |
| | 25 mm × 10 μm | | min |
| PM296 | Phenomenex Gemini 150 × 25 | water (0.05% ammonium | 15%- |
| | mm × 10 μm | hydroxide)-ACN | 45%, 12 min |
| PM297 | Phenomenex Synergi C18 150 × | water (0.05% HCl)-ACN | 13%-39%, 8 |
| | 25 mm × 10 μm | | min |
| PM298 | Phenomenex Synergi C18 150 × | water (0.05% HCl)-ACN | 20%-40%, 9 |
| | 25 mm × 10 μm | | min |
| PM299 | Phenomenex Luna C18 150 × 25 | water (0.1% TFA)-ACN | 7%-37%, 10 |
| | mm × 10 μm | | min |
| PM300 | Waters Vinridis Silica 2-EP OBD | Hexane-EtOH | 15%-55%, |
| | 50 mm × 150 mm × 5 mm | | 15 min |
| PM301 | Phenomenex Luna C18 150 × 25 | water (0.1% TFA)-ACN | 15%-55%, |
| | mm × 10 μm | | 10 min |
| PM302 | Welch Ultimate XB-SiOH 250 × | Hexane-EtOH (0.1% | 20%-60%, |
| | 50 mm × 10 mm | NH3•H2O) | 15 min |
| PM303 | Welch Ultimate XB-CN 250 × 70 | Hexane-EtOH (0.1% | 20%-60%, |
| | mm × 10 mm | NH3•H2O) | 15 min |
| PM304 | Phenomenex Gemini-NX C18 | water (10 mM NH4HCO3) - | 19%-49%, 8 |
| | 75 × 30 mm × 3 mm | ACN | min |
| PM305 | Phenomenex Luna C18 150 × 25 | water (0.1% TFA)-ACN | 12%-42%, 9 |
| | mm × 10 μm | | min |
| PM306 | Welch Xtimate C18 150 × | water (0.05% ammonium | 20%-30%, 7 |
| | 40 mm × 10 mm | hydroxide)-ACN | min |
| PM307 | Welch Xtimate C18 150 × | water (0.05% ammonium | 38%-68%, |
| | 40 mm × 10 mm | hydroxide)-ACN | 10 min |
| PM308 | Waters Xbridge 150 mm × 25 | water (0.05% ammonium | 37%-57%, |
| | mm × 10 μm | hydroxide)-ACN | 10 min |
| PM309 | Phenomenex Luna C18 150 × 25 | water (0.1% TFA)-ACN | 22%-42%, |
| | mm × 10 μm | | 10 min |
| PM310 | Phenomenex Luna C18 150 × 25 | water (0.1% TFA)-ACN | 20%-50%, |
| | mm × 10 μm | | 10 min |
| PM311 | Phenomenex Gemini-NX C18 | water (0.1% TFA)-ACN | 32%-42%, 7 |
| | 75 × 30 mm × 3 mm | | min |
| PM312 | Phenomenex Gemini-NX C18 | water (0.1% TFA)-ACN | 30%-40%, 7 |
| | 75 × 30 mm × 3 mm | | min |
| PM313 | Phenomenex Luna C18 150 × 40 | water (0.225% FA)-ACN | 18%-48%, |
| | mm × 15 μm | | 10 min |
| PM314 | Phenomenex Gemini-NX C18 | water (0.1% TFA)-ACN | 38%-48%, 7 |
| | 75 × 30 mm × 3 mm | | min |
| PM315 | Phenomenex Luna C18 150 × 40 | water (0.1% TFA)-ACN | 30%-50%, |
| | mm × 15 μm | | 10 min |
| PM316 | Waters Xbridge 150 mm × 25 | water (0.05% ammonium | 40%-70%, |
| | mm × 5 μm | hydroxide)-ACN | 10 min |
| PM317 | Phenomenex Gemini-NX C18 | water (0.1% TFA)-ACN | 28%-38%, |
| | 75 × 30 mm × 3 mm | | 7 min |
| PM318 | Phenomenex Synergi C18 150 × | water (0.225% FA)-ACN | 28%-58%, |
| | 25 mm × 10 μm | | 8.5 min |
| PM319 | Phenomenex Gemini-NX C18 | water (0.05% ammonium | 18%-48%, 7 |
| | 75 × 30 mm × 3 mm | hydroxide)-ACN | min |
| PM320 | Waters Xbridge 150 mm × 25 | water (0.05% ammonium | 20%-50%, |
| | mm × 10 μm | hydroxide)-ACN | 11 min |
| PM321 | Phenomenex Gemini-NX C18 | water (0.05% ammonium | 24%-54%, 7 |
| | 75 × 30 mm × 3 mm | hydroxide)-ACN | min |
| PM322 | Welch Ultimate XB-SiOH 250 × | Hexane-EtOH (0.1% | 30%-70%, |
| | 50 mm × 10 mm | NH3•H2O) | 15 min |
| PM323 | Phenomenex Gemini-NX C18 | water (0.05% ammonium | 17%-47%, 7 |
| | 75 × 30 mm × 3 mm | hydroxide)-ACN | min |
| PM324 | Phenomenex Gemini-NX C18 | water (0.05% ammonium | 20%-50%, 7 |
| | 75 × 30 mm × 3 mm | hydroxide)-ACN | min |
| PM325 | Phenomenex Luna C18 150 × 40 | water (0.1% NH3•H2O)- | 30%-70%, |
| | mm × 15 μm | ACN | 15 min |
| PM326 | Phenomenex Gemini-NX C18 | water (0.05% ammonium | 16%-46%, 7 |
| | 75 × 30 mm × 3 mm | hydroxide)-ACN | min |
| PM327 | Welch Ultimate XB-CN 250 × 70 | Hexane-EtOH (0.1% | 15%-55%, |
| | mm × 10 mm | NH3•H2O) | 15 min |
| PM328 | Phenomenex Gemini-NX C18 | water (0.05% ammonium | 8%-38%, 5 |
| | 75 × 30 mm × 3 mm | hydroxide)-ACN | min |
| PM329 | Welch Ultimate XB-CN 250 × 70 | Hexane-EtOH (0.1% | 10%-50%, |
| | mm × 10 mm | NH3•H2O) | 10 min |
| PM330 | Phenomenex Gemini-NX C18 | water (0.05% ammonium | 22%-52%, 7 |
| | 75 × 30 mm × 3 mm | hydroxide)-ACN | min |
| PM331 | Phenomenex Gemini-NX C18 | water (0.05% ammonium | 23%-53%, |
| | 75 × 30 mm × 3 mm | hydroxide)-ACN | 11.5 min |
| PM332 | Unisil 3-100 C18 Ultra 150 × 50 | water (0.225% FA)-ACN | 15%-35%, |
| | mm × 3 mm | | 10 min |
| PM333 | Phenomenex Gemini-NX C18 | water (0.05% ammonium | 7%-37%, 7 |
| | 75 × 30 mm × 3 mm | hydroxide)-ACN | min |
| PM334 | Phenomenex Gemini-NX C18 | water (10 mM NH4HCO3) - | 15%-45%, 8 |
| | 75 × 30 mm × 3 mm | ACN | min |
| PM335 | Phenomenex Gemini-NX C18 | water (0.05% ammonium | 3%-33%, 7 |
| | 75 × 30 mm × 3 mm | hydroxide)-ACN | min |
| PM336 | Phenomenex Gemini-NX C18 | water (10 mM NH4HCO3) - | 14%-44%, 8 |
| | 75 × 30 mm × 3 mm | ACN | min |
| PM337 | Phenomenex Gemini-NX C18 | water (0.05% ammonium | 7%-37%, |
| | 75 × 30 mm × 3 mm | hydroxide)-ACN | 11.5 min |
| PM338 | Waters Xbridge 150 mm × 25 | water (10 mM NH4HCO3) - | 28%-58%, |
| | mm × 10 μm | ACN | 11 min |
| PM339 | SepaFlash SW040 spherical | water (0.1% NH3•H2O)- | 35%-60%, |
| | C18 20-45 mm, 100 A | ACN | 15 min |
| PM340 | SepaFlash SW040 spherical | water (0.1% TFA)-ACN | 30%-55%, |
| | C18 20-45 mm, 100 A | | 10 min |
| PM341 | Phenomenex Synergi C18 150 × | water (10 mM NH4HCO3) - | 55%-70%, |
| | 25 mm × 10 μm | ACN | 15 min |
| PM342 | SepaFlash SW040 spherical | water (0.1% NH3•H2O)- | 32%-50%, |
| | C18 20-45 mm, 100 A | ACN | 15 min |
| PM343 | Welch Xtimate C18 150 × | water (0.1% NH3•H2O)- | 45%-75%, 8 |
| | 40 mm × 10 mm | ACN | min |
| PM344 | SepaFlash SW040 spherical | water (0.1% NH3•H2O)- | 32%-45%, |
| | C18 20-45 mm, 100 A | ACN | 15 min |
| PM345 | Phenomenex Synergi C18 150 × | water (10 mM NH4HCO3) - | 28%-58%, 8 |
| | 25 mm × 10 μm | ACN | min |
| PM346 | SepaFlash SW040 spherical | water (0.1% TFA)-ACN | 40%-50%, |
| | C18 20-45 mm, 100 A | | 10 min |
| PM347 | Welch Xtimate C18 150 × | water (0.1% NH3•H2O)- | 28%-58%, 8 |
| | 40 mm × 10 mm | ACN | min |
| PM348 | SepaFlash SW040 spherical | water (0.1% NH3•H2O)- | 53%-65%, |
| | C18 20-45 mm, 100 A | ACN | 10 min |
| PM349 | Waters Xbridge 150 mm × 25 | water (10 mM NH4HCO3) - | 34%-64%, 9 |
| | mm × 10 μm | ACN | min |
| PM350 | Waters Xbridge 150 mm × 25 | water (10 mM NH4HCO3) - | 31%-61%, 9 |
| | mm × 10 μm | ACN | min |
| PM351 | Waters Xbridge 150 mm × 25 | water (10 mM NH4HCO3) - | 24%-54%, 9 |
| | mm × 10 μm | ACN | min |
| PM352 | Waters Xbridge 150 mm × 25 | water (10 mM NH4HCO3) - | 45%-75%, 8 |
| | mm × 10 μm | ACN | min |
| PM353 | SepaFlash SW040 spherical | water (0.1% NH3•H2O)- | 35%-52%, |
| | C18 20-45 mm, 100 A | ACN | 15 min |
| PM354 | SepaFlash SW040 spherical | water (0.1% NH3•H2O)- | 45%-70%, |
| | C18 20-45 mm, 100 A | ACN | 10 min |
| PM355 | Welch Xtimate C18 150 × | water (0.1% NH3•H2O)- | 25%-55%, 8 |
| | 40 mm × 10 mm | ACN | min |
| PM356 | Waters Xbridge 150 mm × 25 | water (10 mM NH4HCO3) - | 32%-62%, 9 |
| | mm × 10 μm | ACN | min |
| PM357 | Welch Xtimate C18 150 × | water (0.05% ammonium | 31%-61%, 2 |
| | 25 mm × 5 mm | hydroxide)-ACN | min |
| PM358 | Welch Xtimate C18 150 × | water (0.1% TFA)-ACN | 10%-40%, |
| | 25 mm × 5 mm | | 10 min |
| PM359 | Welch Xtimate C18 150 × | water (0.1% TFA)-ACN | 15%-45%, |
| | 25 mm × 5 mm | | 10 min |
| PM360 | Welch Xtimate C18 150 × | water (10 mM NH4HCO3) - | 24%-54%, 2 |
| | 25 mm × 5 mm | ACN | min |
| PM361 | Welch Xtimate C18 150 × | water (0.1% TFA)-ACN | 15%-35%, |
| | 25 mm × 5 mm | | 10 min |
| PM362 | Welch Ultimate C18 150 × 25 | water (0.1% TFA)-ACN | 20%-50%, |
| | mm × 5 mm | | 10 min |
| PM363 | Welch Ultimate C18 150 × 25 | water (0.1% TFA)-ACN | 17%-47%, |
| | mm × 5 mm | | 10 min |
| PM364 | Waters Xbridge 150 mm × 25 | water (10 mM NH4HCO3) - | 22%-52%, |
| | mm × 10 μm | ACN | 10 min |
| PM365 | Welch Xtimate C18 150 × | water (0.1% NH3•H2O)- | 30%- 60%, 8 |
| | 25 mm × 5 mm | ACN | min |
| PM366 | SepaFlash SW040 spherical | water (0.1% TFA)-ACN | 38%-53%, |
| | C18 20-45 mm, 100 A | | 15 min |
| PM367 | YMC Triart C18 150 × 25 mm × | water (0.1% TFA)-ACN | 33%-53%, |
| | 5 mm | | 10 min |
| PM368 | Waters Xbridge 150 mm × 25 | water (10 mM NH4HCO3) - | 35%-65%, 9 |
| | mm × 10 μm | ACN | min |
| PM369 | Waters Xbridge 150 mm × 25 | water (10 mM NH4HCO3) - | 36%-66%, 9 |
| | mm × 10 μm | ACN | min |
| PM370 | Welch Xtimate C18 150 × | water (0.1% NH3•H2O)- | 21%-51%, 2 |
| | 25 mm × 5 mm | ACN | min |

### Abbreviations

Wherein the following abbreviations have been used, the following meanings apply:
ACN is acetonitrile,
AcOH is acetic acid,
AM is analytical method,
aq. is aqueous,
atm. is atmosphere,
Boc₂O is di-tert-butyl dicarbonate,
B₂pin₂ is 4,4,4′,4′,5,5,5′,5′-Octamethyl-2,2′-bi-1,3,2-dioxaborolane,
Brettphos G3 Pd is [(2-Di-cyclohexylphosphino-3,6-dimethoxy-2′,4′,6′-triisopropyl-1,1′-biphenyl)-2-(2′-amino-1,1′-biphenyl)]palladium(II) methanesulfonate methanesulfonate,
CDI is 1,1′-carbonyldiimidazole,
CHCl₃-d₆ is deuterated chloroform,
CO is carbon monoxide gas,
Cs₂CO₃ is cesium carbonate,
CuI is copper iodide,
DCE is dichloroethane,
DCM is dichloromethane,
DHP is 3,4-dihydropyran,
DIPEA is N,N-diisopropylethylamine,
DMAP is dimethylaminopyridine,
DME is 1,2-dimethoxyethane,
DMF is N,N-dimethylformamide,
DMF-DMA is N,N-dimethylformamide dimethyl acetal,
DMP is Dess-Martin periodinane,
DMS is dimethylsulfide,
DMSO is dimethyl sulfoxide,
DMSO-d₆ is deuterated dimethyl sulfoxide,
DPPF is 1,1′-ferrocenediyl-bis(diphenylphosphine),
EA is ethyl acetate,
EDCI is N-(3-dimethylaminopropyl)-N′-ethylcarbodiimide hydrochloride,
EtOH is ethanol,
FA is formic acid,
Fmoc is 9-fluorenylmethoxycarbonyl,
h is hours,
NMR is nuclear magnetic resonance,
HATU is (1-[bis(dimethylamino)methylene]-1H-1,2,3-triazolo[4,5-b]pyridinium 3-oxide hexafluorophosphate,
HCl is hydrochloric acid,
HOBt is 1-hydroxybenzotriazole,
H₂ is hydrogen gas,
H₂O is water,
HPLC is high performance liquid chromatography,
KF is potassium fluoride,
K₂CO₃ is potassium carbonate,
K₃PO₄ is tripotassium phosphate,
K₂SO₄ is potassium sulphate,
LAH is lithium aluminum hydride,
LCMS is Liquid Chromatography Mass Spectrometry,
LDA is lithium diisopropylamide,
LiOH·H₂O is lithium hydroxide monohydrate,
mCPBA is meta-chloroperoxybenzoic acid,
Mel is methyl iodide,
MeOH is methanol,
MeOH-d₄ is deuterated methanol,
min is minutes,
MnO₂ is manganese dioxide,
MS are molecular sieves,
MTBE is methyltertbutylether,
N₂ is nitrogen gas,
NaBH(AcO)₃ is sodium triacetoxyborohydride,
NaBH₄ is sodium borohydride,
NaBH₃CN is sodium cyanoborohydride,
Na₂CO₃ is sodium carbonate,
NaH is sodium hydride,
NaHCO₃ is sodium bicarbonate,
NaN₃ is sodium azide,
NaOH is sodium hydroxide,
NaOMe is sodium methoxide,
Na₂SO₄ is anhydrous sodium sulfate,
NH₃ is ammonia,
NH₄Cl is ammonium chloride,
NCS is N-chlorosuccinimide,
Pd/C is palladium on carbon,
Pd₂(dba)₃ is tris(dibenzylideneacetone)dipalladium(0),
Pd(PPh₃)₄ is tetrakis(triphenylphosphine)palladium(0),
Pd(dppf)Cl₂ is [1,1′-bis(diphenylphosphino)ferrocene]dichloropalladium(II),
Pd(dppf)Cl₂·CHCl₂ is [1,1′-bis(diphenylphosphino)ferrocene]dichloropalladium(II), complex with dichloromethane,
PE is petroleum ether,
PM is purification method,
PMB is p-methoxybenzyl,
POCl₃ is phosphorous oxychloride,
PPh₃ is triphenylphosphine,
RT is room temperature,
rt is retention time,
SEM is silylethoxymethyl,
SOCl₂ is thionyl chloride,
SiO₂ is silica,
TBAF is tetrabutylammonium fluoride,
TBAI is tetramethylammonium iodide,
TEA is triethylamine,
TFA is trifluoroacetic acid,
TFAA is trifluoroacetic anhydride,
THF is tetrahydrofuran,
TLC is thin layer chromatography,
TMSCl is trimethylsilyl chloride,
T3P is propylphosphonic anhydride,
Trt is trityl,
TrtCl is trityl chloride; and
TsOH·H₂O is p-toluenesulfonic acid monohydrate.

### Preparation of Intermediates

The following Preparations describe the methods used for common intermediates required for synthesis of the Examples.

### Synthesis of Intermediate D

### 3-(((2-chloro-[1,1′-biphenyl]-4-yl)methyl)amino)propanoic acid, 1.39

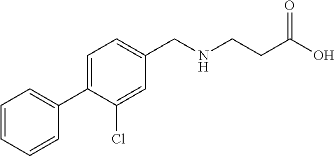 [SMILES: O=C(O)CCNCc1ccc(-c2ccccc2)c(Cl)c1]

A solution of 3-Chloro-4-phenylbenzaldehyde (CAS 57592-44-6, 10 g, 46.15 mmol) (2-chloro-[1,1′-biphenyl]-4-carbaldehyde (Bioorganic and Medicinal Chemistry, 2017, 25 (13), 3471-3482)) and 3-aminopropanoic acid (12.34 g, 138.46 mmol) in MeOH (200 mL) was stirred at 34° C. for 1 h. NaBH(AcO)₃ (19.56 g, 92.31 mmol) was added slowly and the reaction mixture was stirred at 34° C. for 14 h. The reaction mixture was filtered and concentrated in vacuo to give a residue that was purified (PM64) to afford compound 1.39 (11.9 g, 41.07 mmol, 89% yield) as a colourless oil.

LCMS (AM3): rt=0.758 min, (290.1 [M+H]⁺), 50% purity.

### 3-((tert-butoxycarbonyl)((2-chloro-[1,1′-biphenyl]-4-yl)methyl)amino)propanoic acid, Intermediate D

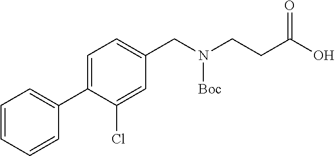 [SMILES: CC(C)(C)OC(=O)N(CCC(=O)O)Cc1ccc(-c2ccccc2)c(Cl)c1]

To a solution of compound 1.39 (11.9 g, 41.07 mmol) in THF (75 mL) and H₂O (75 mL) was added NaHCO₃ (5.18 g, 61.60 mmol) and (Boc)₂O (10.76 g, 49.28 mmol) sequentially at 30° C., the mixture was stirred for 2 h. The reaction mixture was filtered and concentrated in vacuo, and the residue was purified (PM64) to afford Intermediate D (13.8 g, 35.40 mmol, 86.2% yield) as a yellow oil.

LCMS (AM3): rt=0.997 min, (412.1 [M+Na]⁺). 92% purity.

### Synthesis of Intermediate H

### tert-butyl ((2-chloro-[1,1′-biphenyl]-4-yl)methyl)(3-((3,3-diethoxypropyl)amino)-3-oxopropyl)carbamate 1.77

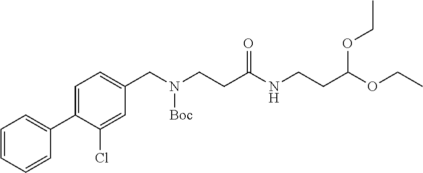 [SMILES: CCOC(CCNC(=O)CCN(Cc1ccc(-c2ccccc2)c(Cl)c1)C(=O)OC(C)(C)C)OCC]

To a solution of Intermediate D (10 g, 25.65 mmol), 3,3-diethoxypropan-1-amine (4.53 g, 30.78 mmol) and TEA (10.71 mL, 76.95 mmol) in THF (100 mL) was added T₃P (24.48 g, 38.47 mmol, 50% in EA) at 0° C. The resulting mixture was stirred at 0° C. for 2 h. The mixture was diluted with water (500 mL) and extracted with EA (200 mL×2). The combined organic phases were washed (brine, 200 mL), dried (Na₂SO₄) and concentrated in vacuo. The residue was purified (PM5) to give compound 1.77 (11.2 g, 21.58 mmol, 84.1% yield) as a colourless oil.

LCMS (AM3): rt=1.057 min, (541.2 [M+Na]⁺), 76% purity.

### tert-butyl ((2-chloro-[1,1′-biphenyl]-4-yl)methyl)(3-oxo-3-((3-oxopropyl)amino)propyl)carbamate; Intermediate H

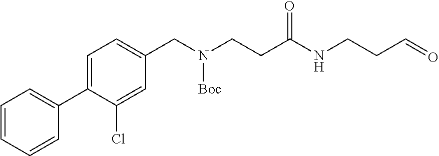 [SMILES: CC(C)(C)OC(=O)N(CCC(=O)NCCC=O)Cc1ccc(-c2ccccc2)c(Cl)c1]

A solution of compound 1.77 (1 g, 1.93 mmol) in AcOH (5 mL) and water (5 mL) was stirred at 30° C. for 2. The mixture was basified with sat. NaHCO₃ (aq.) to pH=8 and then extracted with EA (50 mL×2). The combined organic phases were washed (brine, 50 mL), dried (Na₂SO₄) and concentrated in vacuo to give Intermediate H (850 mg, 1.91 mmol, 99.1% yield) as a colourless oil, which was used directly without further purification.

LCMS (AM3): rt=0.961 min, (467.2 [M+Na]⁺), 86.8% purity.

### Synthesis of Intermediate N

### N-(3-((6-(4H-1,2,4-triazol-4-yl)-1H-indazol-4-yl)amino)propyl)-3-aminopropanamide; Intermediate N

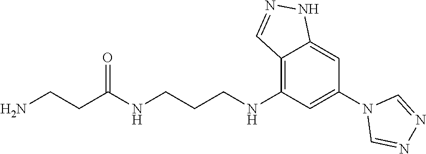 [SMILES: NCCC(=O)NCCCNc1cc(-n2cnnc2)cc2[nH]ncc12]

A mixture of compound 1.279 (450 mg, 1.09 mmol) in a solution of HCl in MeOH (4 M, 10 mL) was stirred at 20° C. for 1 h. The mixture was concentrated in vacuo and the residue was purified (PM69) to afford Intermediate N (350 mg, 0.959 mmol, 87.9% yield, HCl salt) as a yellow solid.

LCMS (AM4): rt=0.701 min, (329.1 [M+H]⁺), 98.3% purity.

### Synthesis of Intermediate I

### tert-Butyl 3-chloro-4-(trifluoromethoxy)benzyl(4-oxobutyl)carbamate, Intermediate I

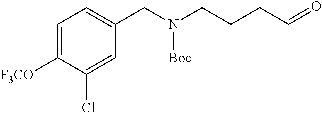 [SMILES: CC(C)(C)OC(=O)N(CCCC=O)Cc1ccc(OC(F)(F)F)c(Cl)c1]

To a solution of tert-Butyl 3-chloro-4-(trifluoromethoxy)benzyl(4-hydroxybutyl)carbamate (WO2022185041, 1.45 g, 3.64 mmol) in DCM (10 mL) was added DMP (1.86 g, 4.37 mmol) at 20° C. The resulting mixture was stirred at 20° C. for 1 h. The mixture was concentrated in vacuo and purified (PM7) to afford Intermediate 1 (1.15 g, 2.91 mmol, 79.7% yield) as a light-yellow oil.

LCMS (AM3): rt=1.036 min, (418.1 [M+Na]⁺), 72.2% purity.

### Synthesis of Intermediate J

### N-((2-Chloro-[1,1′-biphenyl]-4-yl)methyl)but-3-yn-1-amine, 1.169

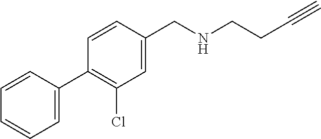 [SMILES: C#CCCNCc1ccc(-c2ccccc2)c(Cl)c1]

A mixture of but-3-yn-1-amine hydrochloride (974.47 mg, 9.23 mmol, HCl salt), NaOAc (946.56 mg, 11.54 mmol) and 3-Chloro-4-phenylbenzaldehyde (CAS 57592-44-6, 500 mg, 2.31 mmol) in MeOH (20 mL) was stirred at 20° C. for 12 h, then NaBH(AcO)₃ (1.96 g, 9.23 mmol) was added. The mixture was stirred at 20° C. for 3 h. The mixture was filtered, and the filtrate was concentrated in vacuo. The residue was purified (PM61) to afford compound 1.169 (500 mg, 1.85 mmol, 80.3% yield) as a yellow oil.

LCMS (AM3): rt=0.794 min, (270.5 [M+H]⁺), 100% purity.

### tert-Butyl but-3-yn-1-yl((2-chloro-[1,1′-biphenyl]-4-yl)methyl)carbamate, Intermediate J

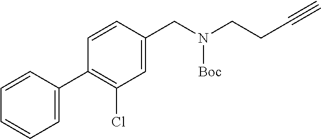 [SMILES: C#CCCN(Cc1ccc(-c2ccccc2)c(Cl)c1)C(=O)OC(C)(C)C]

To a mixture of compound 1.169 (500 mg, 1.85 mmol) and TEA (562.65 mg, 5.56 mmol) in THF (10 mL) was added Boc₂O (444.96 mg, 2.04 mmol) at 20° C. The mixture was stirred at 20° C. for 12 h. The mixture was concentrated in vacuo and purified (PM6) to afford Intermediate J (370 mg, 1.00 mmol, 54% yield) as a colorless oil.

¹H NMR (400 MHz, CHCl₃-dCHCl₃-d) δ: 7.48-7.29 (m, 7H), 7.19 (br s, 1H), 4.55 (s, 2H), 3.51-3.33 (m, 2H), 2.52-2.40 (m, 2H), 2.00 (t, J=2.4 Hz, 1H), 1.54-1.45 (m, 9H) ppm.

### Synthesis of Intermediate 1.3

### 2-(4-amino-1-(tetrahydro-2H-pyran-2-yl)-1H-indazol-6-yl)acetic acid 1.2

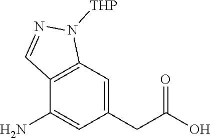

To a mixture of Pd/C (100 mg, 10% purity) in MeOH (40 mL) was added compound 2.449 (270 mg, 884.42 μmol). The mixture was degassed and purged with H₂ (×3), then the mixture was stirred at 20° C. for 1 h under H₂ (15 psi). The mixture was filtered and the filtrate was concentrated in vacuo to afford compound 1.2 (126 mg, 457.68 μmol, 51.75% yield) as a brown gum.

LCMS (AM11): rt=0.300 min, (297.8 [M+Na]⁺), 76.51% purity.

### 2-(4-((2-(4-((tert-butoxycarbonyl)(3,5-difluoro-4-(trifluoromethoxy)benzyl)amino)butoxy)ethyl)amino)-1-(tetrahydro-2H-pyran-2-yl)-1H-indazol-6-yl)acetic acid 1.3

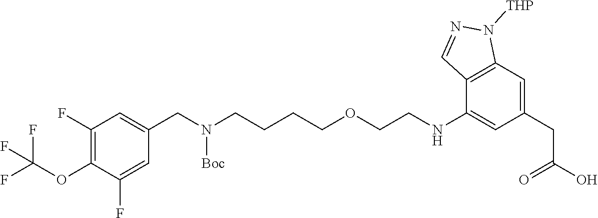

To a mixture of compound 1.941 (130 mg, 294.53 μmol) and compound 1.2 (117.36 mg, 323.98 μmol) in MeOH (4 mL) was added MgSO₄ (177.26 mg, 1.47 mmol) and AcOH (26.53 mg, 441.79 μmol). The mixture was stirred at 20° C. for 12 h, then NaBH₃CN (37.02 mg, 589.05 mol) was added. The mixture was stirred at 20° C. for 1 h. The reaction mixture was poured into water (20 mL) and extracted with EA (20 mL×3). The combined organic phase was washed (brine, 20 mL×2), dried (Na₂SO₄), filtered and concentrated in vacuo to give a residue, which was purified (PM70) to afford compound 1.3 (120 mg, 162.01 μmol, 55.01% yield) as a yellow gum.

LCMS (AM11): rt=0.527 min, (701.1, [M+H]⁺), 94.60% purity.

### Synthesis of Intermediate 1.7

### 4-(((2-chloro-[1,1′-biphenyl]-4-yl)methyl)amino)butan-1-ol 1.5

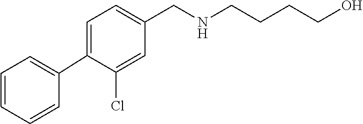 [SMILES: OCCCCNCc1ccc(-c2ccccc2)c(Cl)c1]

A mixture of 2-chloro-[1,1′-biphenyl]-4-carbaldehyde (10 g, 42.00 mmol) (Bioorganic and Medicinal Chemistry, 2017, 25 (13), 3471-3482), 4-aminobutan-1-ol (8.01 g, 89.88 mmol) and 4 Å MS (20 g) in MeOH (200 mL) was stirred at RT for 18 h, and then NaBH₃CN (9 g, 143.22 mmol) added. The mixture was stirred for 4 h at RT. The reaction mixture was filtered, the filtrate was concentrated in vacuo to give a residue that was added to H₂O (200 mL) and the aq. mixture was extracted with EA (200 mL×3). The combined organic phases were washed (brine, 300 mL), dried (Na₂SO₄), filtered and concentrated in vacuo. The crude product was purified (PM19) to give compound 1.5 (6.9 g, 23.77 mmol, 51.6% yield) as a light-brown gum.

¹H NMR (CHCl₃-dCHCl₃-d, 400 MHz) δ: 7.37-7.34 (m, 6H), 7.25-7.19 (m, 2H), 3.74 (s, 2H), 3.56 (m, 2H), 3.38 (bs, 2H), 2.68 (t, J=5.6 Hz, 2H), 1.63-1.58 (m, 4H) ppm.

### tert-butyl ((2-chloro-[1,1′-biphenyl]-4-yl)methyl)(4-hydroxybutyl)carbamate 1.6

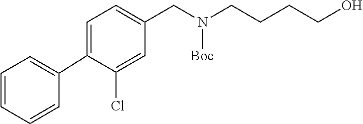 [SMILES: CC(C)(C)OC(=O)N(CCCCO)Cc1ccc(-c2ccccc2)c(Cl)c1]

To a mixture of compound 1.5 (6.9 g, 23.77 mmol) in THF (45 mL) and H₂O (15 mL) was added NaHCO₃ (4.00 g, 47.62 mmol) and (Boc)₂O (6 g, 27.49 mmol) at RT. The mixture was stirred for 18 h. To the reaction mixture was added H₂O (100 mL), extracted with EA (100 mL×3) and the combined organic phases washed (brine, 100 mL), dried (Na₂SO₄), filtered and concentrated in vacuo. The residue was (PM20) to afford compound 1.6 (7.0 g, 17.99 mmol, 75% yield) as a light-brown oil.

LCMS (AM1): rt=0.839 min, (334.1 [M-^{t}Bu+2H]⁺), 99.3% purity.

### tert-butyl ((2-chloro-[1,11′-biphenyl]-4-yl)methyl)(4-oxobutyl)carbamate 1.7

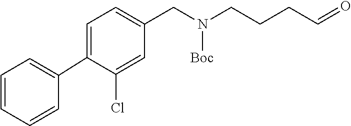 [SMILES: CC(C)(C)OC(=O)N(CCCC=O)Cc1ccc(-c2ccccc2)c(Cl)c1]

To a solution of compound 1.6 (1.5 g, 3.85 mmol) in DCM (30 mL) was added DMP (2 g, 4.72 mmol) at RT. The reaction mixture was then stirred for 15 h. The reaction mixture filtered, the filtrate concentrated in vacuo and purified (PM21) to afford compound 1.7 (1.01 g, 62.9% yield) as a colourless oil.

### Synthesis of Intermediate 1.67

### (4-Chloro-5-phenyl-1H-pyrazol-3-yl)methanol 1.66

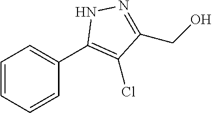 [SMILES: OCc1n[nH]c(-c2ccccc2)c1Cl]

To a mixture of 5-phenyl-1H-pyrazol-3-yl methanol (800 mg, 4.59 mmol) (Journal of Medicinal Chemistry, 1998, 41(13), 2390-2410) in ACN (10 mL) was added NCS (919.87 mg, 6.89 mmol) under N₂ at 25° C. The mixture was stirred at 25° C. for 12 h. The mixture was filtered and the filtrate was concentrated in vacuo to give a residue. The obtained residue was purified (PM65) to afford compound 1.66 (250 mg, 1.14 mmol, 24.8% yield, 95% purity) as a white solid.

LCMS (AM3): rt=0.813 min, (209.1 [M+H]⁺), 94.39% purity.

### 4-Chloro-5-phenyl-1H-pyrazole-3-carbaldehyde 1.67

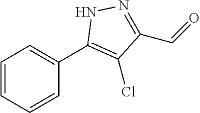 [SMILES: O=Cc1n[nH]c(-c2ccccc2)c1Cl]

To a mixture of compound 1.66 (250 mg, 1.20 mmol) in DCM (10 mL) was added manganese (IV) oxide (1.04 g, 11.98 mmol) under N₂ at 25° C. The mixture was stirred at 25° C. for 12 h. The mixture was filtered and the filtrate was concentrated in vacuo to give a residue which was further purified (PM66) to afford compound 1.67 (60 mg, 281.67 mol, 23.5% yield, 97% purity) as a white solid.

LCMS (AM3): rt=0.858 min, (207.0 [M+H]⁺), 96.49% purity.

### Synthesis of Intermediate 1.81

### Methyl 4-bromo-1-(tetrahydro-2H-pyran-2-yl)-1H-indazole-6-carboxylate 1.79

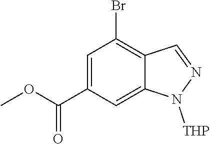

To a mixture of Methyl 4-bromo-1H-indazole-6-carboxylate (CAS 885518-47-8, 2 g, 7.84 mmol) in THF (20 mL) was added 3,4-dihydro-2H-pyran (1.32 g, 15.68 mmol, 1.43 mL) and 4-methylbenzenesulfonic acid (149 mg, 0.784 mmol) at RT. The mixture was heated to 50° C. and stirred for 12 h. The mixture was diluted with saturated aq. NaHCO₃ (30 mL) and extracted with EA (30 mL×3). The combined organic phases were washed (brine, 150 mL), dried (Na₂SO₄), filtered and concentrated in vacuo. The residue was purified (PM14) to afford compound 1.79 (2.4 g, 7.08 mmol, 90.2% yield) as a white solid.

¹H NMR (400 MHz, CHCl₃-dCHCl₃-d) δ 8.30 (s, 1H), 8.08 (s, 1H), 8.00 (d, J=1.2 Hz, 1H), 5.78 (dd, J=9.2, 2.8 Hz, 1H), 4.07-3.95 (m, 4H), 3.83-3.73 (m, 1H), 2.60-2.44 (m, 1H), 2.20-2.07 (m, 2H), 1.82-1.70 (m, 3H) ppm.

### Methyl 4-amino-1-(tetrahydro-2H-pyran-2-yl)-1H-indazole-6-carboxylate 1.80

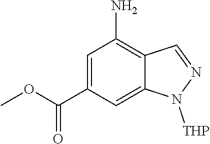

To a mixture of compound 1.79 (1.9 g, 5.60 mmol) in DMSO (30 mL) was added ammonium hydroxide (2.07 g, 14.8 mmol), CuI (224 mg, 1.18 mmol), (2S,4S)-4-hydroxypyrrolidine-2-carboxylic acid (301 mg, 2.30 mmol) and K₂CO₃ (2.39 g, 17.31 mmol) sequentially at RT under N₂. The reaction mixture was heated to 90° C. and stirred for 16 h. The mixture was diluted with water (40 mL), extracted with EA (30 mL×3), the combined organic phases were washed (brine, 50 mL×3), dried (Na₂SO₄), filtered and concentrated in vacuo to afford compound 1.80 (1.2 g, 4.36 mmol, 77.8% yield) as a yellow solid.

LCMS (AM3): rt=0.727 min, (276.1 [M+H]⁺), 57.3% purity.

### Methyl 4-((3-(3-((tert-butoxycarbonyl)((2-chloro-[1,1′-biphenyl]-4-yl)methyl)amino)propanamido)propyl)amino)-1-(tetrahydro-2H-pyran-2-yl)-1H-indazole-6-carboxylate 1.81

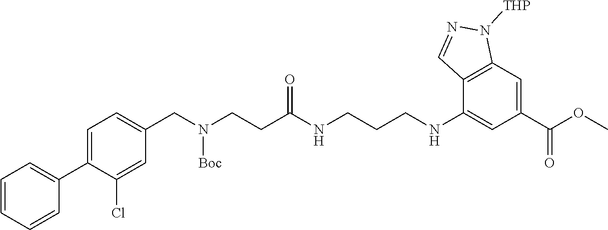

To a mixture of compound 1.80 (1.29 g, 2.91 mmol) and Intermediate H (1 g, 3.63 mmol) in DCE (30 mL) was added NaBH(AcO)₃ (1.54 g, 7.26 mmol) at 25° C. The mixture was stirred at 25° C. for 16 h. The mixture was concentrated in vacuo and the residue was purified (PM71) to afford compound 1.81 (460 mg, 0.653 mmol, 18% yield) as a white solid.

LCMS (AM3): rt=1.096 min, (704.4 [M+H]⁺), 100% purity.

The following Intermediates in Table 1 were made with non-critical changes or substitutions to the exemplified procedure for Intermediate H that would be understood by one skilled in the art, wherein R may or may not contain a THP protecting group.

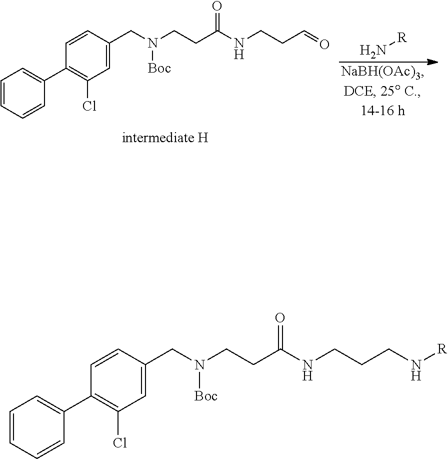

TABLE 1

| | Product Intermediate | |
|---|---|---|
| Reagent H₂N—R IUPAC | IUPAC name/Intermediate | |
| name/Intermediate No. | No. | Analytical |
| 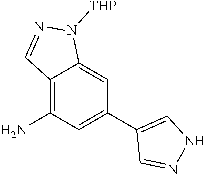 | tert-Butyl (3-((3-((6-(1H- pyrazol-4-yl)-1-(tetrahydro- 2H-pyran-2-yl)-1H-indazol- 4-yl)amino)propyl)amino)- 3-oxopropyl)((2-chloro- [1,1′-biphenyl]-4- yl)methyl)carbamate 1.303 | LCMS (AM3): rt = 0.970 min, (712.4 [M + H]⁺), 65.3% purity. Purification Method PM94 |
| 6-(1H-pyrazol-4-yl)-1- | | |
| (tetrahydro-2H-pyran-2- | | |
| yl)-1H-indazol-4-amine | | |
| 1.302 | | |
| 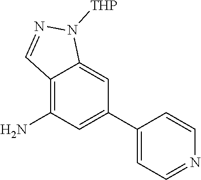 | tert-Butyl ((2-chloro-[1,1′- biphenyl]-4-yl)methyl)(3- oxo-3-((3-((6-(pyridin-4-yl)- 1-(tetahydro-2H-pyran-2- yl)-1H-indazol-4- yl)amino)propyl)amino) propyl)carbamate 1.323 | LCMS (AM3): rt = 0.946 min, (723.3 [M + H]⁺), 97.1% purity. Purification Method PM98 |
| 6-(Pyridin-4-yl)-1- | | |
| (tetrahydro-2H-pyran-2- | | |
| yl)-1H-indazol-4-amine | | |
| 1.322 | | |

### Synthesis of Intermediate 1.83

### 4-((3-(3-((tert-butoxycarbonyl)((2-chloro-[1,1I′-biphenyl]-4-yl)methyl)amino)propanamido)propyl)amino)-1-(tetrahydro-2H-pyran-2-y)-1H-indazole-6-carboxylic acid 1.82

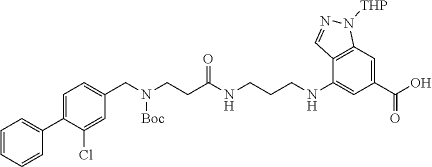

To a mixture of compound 1.81 (360 mg, 0.511 mmol) in THF (18 mL), MeOH (0.2 mL) and water (0.2 mL) was added LiOH·H₂O (214.5 mg, 5.11 mmol) at 25° C. The mixture was stirred at 25° C. for 20 h. The reaction mixture was concentrated in vacuo and the residue was purified (PM72) to afford compound 1.82 (340 mg, 0.493 mmol, 96.4% yield) as a yellow solid.

LCMS (AM3): rt=0.987 min, (690.4 [M+H]⁺), 86.7% purity.

### tert-butyl(3-((3-((6-carbamoyl-1-(tetrahydro-2H-pyran-2-yl)-1H-indazol-4-yl)amino)propyl)amino)-3-oxopropyl)((2-chloro-[1,1′-biphenyl]-4-yl)methyl)carbamate 1.83

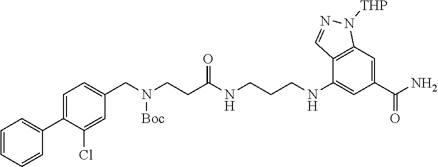

To a mixture of compound 1.82 (150 mg, 0.217 mmol) and NH₄Cl (23.25 mg, 0.435 mmol) in THF (6 mL) was added HATU (99.16 mg, 0.261 mmol) and DIPEA (84.26 mg, 0.652 mmol) at 25° C. The mixture was stirred at 25° C. for 12 h. The mixture was diluted with water (10 mL) and extracted with EA (15 mL×2). The combined organic phase was washed (brine, 30 mL), dried (Na₂SO₄), filtered and concentrated in vacuo to afford compound 1.83 (130 mg) as a yellow oil, which was used directly without further purification.

LCMS (AM3): rt=0.957 min, (689.4 [M+H]⁺), 85% purity.

### Synthesis of Intermediate 1.88

### 4-Bromo-1-(tetrahydro-2H-pyran-2-yl)-1H-indazole-6-carboxylic acid 1.84

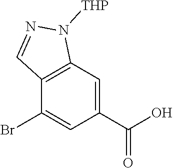

To a solution of compound 1.79 (1.0 g, 2.95 mmol) in THF (10 mL) and water (10 mL) was added LiOH·H₂O (247 mg, 5.9 mmol) at 30° C. The resulting mixture was stirred at 30° C. for 2 h. The solvent was removed in vacuo, the aqueous phase was acidified with aq. HCl (1 M) to pH=4, and then extracted with EA (50 mL×2). The combined organic phases were washed (brine, 50 mL), dried (Na₂SO₄) and concentrated in vacuo to afford compound 1.84 (950 mg) as a white solid.

LCMS (AM3): rt=0.827 min, (239.9 [M+2]⁺), 85% purity.

### 4-Bromo-1-(tetrahydro-2H-pyran-2-yl)-1H-indazole-6-carboxamide 1.85

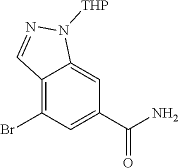

To a solution of compound 1.84 (500 mg, 1.54 mmol) in DMF (10 mL) was added EDCI (590 mg, 3.08 mmol), HOBt (416 mg, 3.08 mmol), DIPEA (397 mg, 3.08 mmol) and NH₄Cl (329 mg, 6.15 mmol) sequentially at 30° C. The resulting mixture was stirred at 30° C. for 15 h. The mixture was diluted with water (50 mL) and extracted with EA (50 mL×2). The combined organic phases were washed (brine, 50 mL), dried (Na₂SO₄) and concentrated in vacuo to afford compound 1.85 (450 mg) as a colourless oil, which was used directly without further purification.

### 4-Bromo-1-(tetrahydro-2H-pyran-2-yl)-1H-indazole-6-carbonitrile 1.86

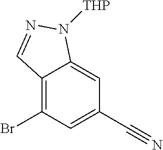

To a solution of compound 1.85 (450 mg, 1.39 mmol) in THF (10 mL) was added Burgess reagent (330.1 mg, 1.39 mmol) at 30° C. The resulting mixture was stirred at 30° C. for 15 h. The solvent was removed in vacuo and the residue dissolved in EA (50 mL). The organic phase was washed (brine, 25 mL), dried (Na₂SO₄) and concentrated in vacuo. The residue was purified (PM7) to afford compound 1.86 (370 mg, 1.21 mmol, 87% yield) as a white solid.

¹H NMR (400 MHz, CHCl₃-dCHCl₃-d) δ 8.12 (s, 1H), 8.02 (d, J=0.8 Hz, 1H), 7.56 (d, J=0.8 Hz, 1H), 5.78 (dd, J=9.2, 2.8 Hz, 1H), 4.05-3.98 (m, 1H), 3.83-3.75 (m, 1H), 2.53-2.42 (m, 1H), 2.20-2.11 (m, 2H), 1.85-1.70 (m, 3H) ppm.

### 4-Amino-1-(tetrahydro-2H-pyran-2-yl)-1H-indazole-6-carbonitrile 1.87

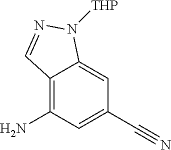

To a mixture of compound 1.86 (320 mg, 1.05 mmol) in DMSO (8 mL) was added aq. NH₃ solution (387 mg, 2.76 mmol), CuI (41.80 mg, 0.219 mmol), (2S,4S)-4-hydroxypyrrolidine-2-carboxylic acid (56.19 mg, 0.429 mmol) and K₂CO₃ (433 mg, 3.14 mmol) sequentially at rt. The reaction mixture was heated to 90° C. and stirred for 16 h under N₂ protection. The reaction mixture was diluted with water (30 mL) and extracted with EA (30 mL×2). The combined organic phases were washed (brine, 50 mL×3), dried (Na₂SO₄), filtered and concentrated in vacuo to afford compound 1.87 (250 mg) as a yellow oil.

LCMS (AM3): rt=0.753 min, (265.1 [M+Na]⁺), 48% purity.

### tert-butyl((2-chloro-[1,1′-biphenyl]-4-yl)methyl)(3-((3-((6-cyano-1-(tetrahydro-2H-pyran-2-yl)-1H-indazol-4-yl)amino)propyl)amino)-3-oxopropyl)carbamate 1.88

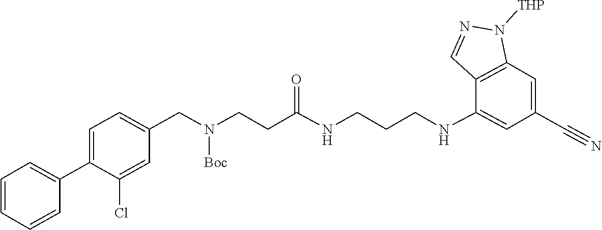

A mixture of compound 1.87 (200 mg, 0.826 mmol) and Intermediate H (367 mg, 0.826 mol in MeOH (10 mL) was stirred at 30° C. for 12 h, then NaBH₃CN (519 mg, 8.26 mmol) was added. The reaction mixture was stirred at 30° C. for 2 h. The reaction mixture was concentrated in vacuo and the residue was purified (PM73) to afford compound 1.88 (200 mg, 0.28 μmol, 33.9% yield) as a white solid.

LCMS (AM2): rt=1.010 min, (671.3 [M+H]⁺), 94% purity.

The following Intermediates in Table 2 were made with non-critical changes or substitutions to the exemplified procedure for Intermediate 1.88 that would be understood by one skilled in the art, wherein R may or may not contain a THP protecting group.

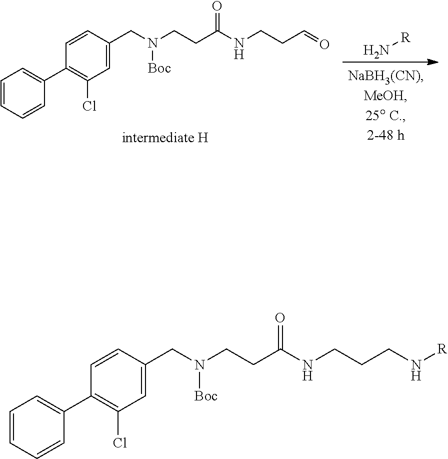

TABLE 2

| Reagent H₂N—R | Product Intermediate | |
|---|---|---|
| IUPAC name/Intermediate | IUPAC name/Intermediate | |
| No. | No. | Analytical |
| 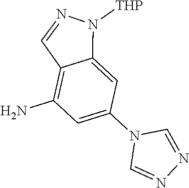 | tert-Butyl ((2-chloro-[1,1′- biphenyl]-4-yl)methyl)(3- oxo-3-((3-((1-(tetrahydro- 2H-pyran-2-yl)-6-(4H- 1,2,4-triazol-4-yl)-1H- indazol-4- yl)amino)propyl)amino) propyl)carbamate 1.274 | LCMS (AM3): rt = 1.037 min, (713.3 [M + H]⁺), 77.9% purity. Purification Method PM74 |
| 1-(Tetrahydro-2H-pyran-2- | | |
| yl)-6-(4H-1,2,4-triazol-4- | | |
| yl)-1H-indazol-4-amine | | |
| 1.273 | | |
| 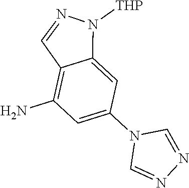 | tert-Butyl ((2-chloro-[1,1′- biphenyl]-4-yl)methyl)(3- oxo-3-((3-((1-(tetrahydro- 2H-pyran-2-yl)-6-(1H- 1,2,4-triazol-1-yl)-1H- indazol-4- yl)amino)propyl)amino) propyl)carbamate 1.295 | LCMS (AM3): rt = 1.080 min, (713.2 [M + H]⁺), 98.7% purity. Purification Method PM84 |
| 1-(Tetrahydro-2H-pyran-2- | | |
| yl)-6-(1H-1,2,4-triazol-1- | | |
| yl)-1H-indazol-4-amine | | |
| 1.294 | | |
| 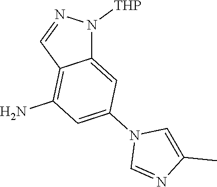 | tert-butyl ((2-chloro-[1,1′- biphenyl]-4-yl)methyl)(3- ((3-((6-(4-methyl-1H- imidazol-1-yl)-1- (tetrahydro-2H-pyran-2-yl)- 1H-indazol-4- yl)amino)propyl)amino)-3- oxopropyl)carbamate 1.904 | LCMS (AM3): rt = 0.935 min, (726.1 [M + H]⁺), 96.2% purity. Purification Method PM114 |
| 6-(4-methyl-1H-imidazol- | | |
| 1-yl)-1-(tetrahydro-2H- | | |
| pyran-2-yl)-1H-indazol-4- | | |
| amine 1.903 | | |
| 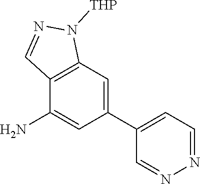 | tert-butyl ((2-chloro-[1,1′- biphenyl]-4-yl)methyl)(3- oxo-3-((3-((6-(pyridazin-4- yl)-1-(tetrahydro-2H-pyran- 2-yl)-1H-indazol-4- yl)amino)propyl)amino) propyl)carbamate 1.924 | LCMS (AM3): rt = 1.136 min, (826.2 [M + H]⁺), 97.1% purity. Purification Method PM120 |
| 6-(pyridazin-4-yl)-1- | | |
| (tetrahydro-2H-pyran-2- | | |
| yl)-1H-indazol-4-amine | | |
| 1.923 | | |
| 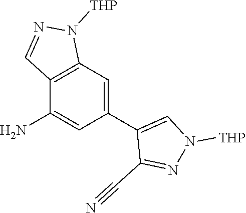 | tert-butyl ((2-chloro-[1,1′- biphenyl]-4-yl)methyl)(3- ((3-((6-(3-cyano-1- (tetrahydro-2H-pyran-2-yl)- 1H-pyrazol-4-yl)-1- (tetrahydro-2H-pyran-2-yl)- 1H-indazol-4- yl)amino)propyl)amino)-3- oxopropyl)carbamate 1.928 | LCMS (AM3): rt = 1.160 min, (821.1 [M + H]⁺), 97.6% purity. Purification Method PM120 |
| 4-(4-amino-1-(tetrahydro- | | |
| 2H-pyran-2-yl)-1H-indazol- | | |
| 6-yl)-1-(tetrahydro-2H- | | |
| pyran-2-yl)-1H-pyrazole-3- | | |
| carbonitrile 1.927 | | |
| 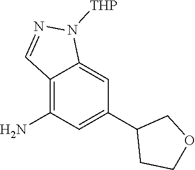 | tert-butyl ((2-chloro-[1,1′- biphenyl]-4-yl)methyl)(3- oxo-3-((3-((1-(tetrahydro- 2H-pyran-2-yl)-6- (tetrahydrofuran-3-yl)-1H- indazol-4- yl)amino)propyl)amino) propyl)carbamate 2.009 | LCMS (AM3): rt = 1.002 min, (716.4 [M + H]⁺), 95.5% purity. Purification Method PM90 |
| 1-(tetrahydro-2H-pyran-2-yl)- | | |
| (tetrahydrofuran-3-yl)-1H- | | |
| indazol-4-amine 2.008 | | |
| 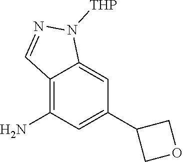 | tert-butyl ((2-chloro-[1,1′- biphenyl]-4-yl)methyl)(3- ((3-((6-(oxetan-3-yl)-1- (tetrahydro-2H-pyran-2-yl)- 1H-indazol-4- yl)amino)propyl)amino)-3- oxopropyl)carbamate 2.011 | LCMS (AM3): rt = 1.070 min, (703.6 [M + H]⁺), 99.5% purity. Purification Method PM103 |
| 6-(oxetan-3-yl)-1- | | |
| (tetrahydro-2H-pyran-2-yl)- | | |
| 1H-indazol-4-amine 2.010 | | |
| 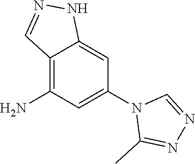 | tert-butyl ((2-chloro-[1,1′- biphenyl]-4-yl)methyl)(3- ((3-((6-(3-methyl-4H-1,2,4- triazol-4-yl)-1H-indazol-4- yl)amino)propyl)amino)-3- oxopropyl)carbamate 2.072 | LCMS (AM3): rt = 0.913 min, (643.2 [M + H]⁺), 100% purity. Purification Method PM152 |
| 6-(3-methyl-4H-1,2,4- | | |
| triazol-4-yl)-1H-indazol-4- | | |
| amine 2.071 | | |
| 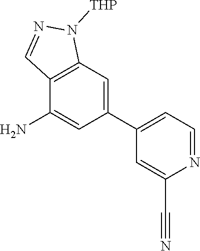 | tert-butyl ((2-chloro-[1,1′- biphenyl]-4-yl)methyl)(3- ((3-((6-(2-cyanopyridin-4- yl)-1-(tetrahydro-2H-pyran- 2-yl)-1H-indazol-4- yl)amino)propyl)amino)-3- oxopropyl)carbamate 2.096 | LCMS (AM3): rt = 1.073 min, (748.2 [M + H]⁺), 96.4% purity Purification Method PM123 |
| 4-(4-amino-1-(tetrahydro- | | |
| 2H-pyran-2-yl)-1H-indazol- | | |
| 6-yl)picolinonitrile 2.095 | | |
| 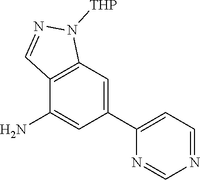 | tert-butyl ((2-chloro-[1,1′- biphenyl]-4-yl)methyl)(3- oxo-3-((3-((6-(pyrimidin-4- yl)-1-(tetrahydro-2H-pyran- 2-yl)-1H-indazol-4- yl)amino)propyl)amino) propyl)carbamate 2.099 | LCMS (AM3): rt = 1.065 min, (724.3 [M + H]⁺), 96.7% purity Purification Method PM124 |
| 6-(pyrimidin-4-yl)-1-(tetrahydro- | | |
| 2H-pyran-2-yl)-1H-indazol-4- | | |
| amine 2.098 | | |
| 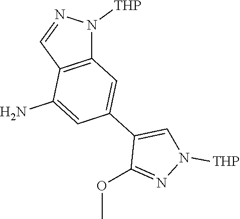 | tert-butyl ((2-chloro-[1,1′- biphenyl]-4-yl)methyl)(3- ((3-((6-(3-methoxy-1- (tetrahydro-2H-pyran-2-yl)- 1H-pyrazol-4-yl)-1- (tetrahydro-2H-pyran-2-yl)- 1H-indazol-4- yl)amino)propyl)amino)-3- oxopropyl)carbamate 2.101 | LCMS (AM3): rt = 1.131 min, (826.3 [M + H]⁺), 94.7% purity Purification Method PM120 |
| 6-(3-methoxy-1-(tetrahydro- | | |
| 2H-pyran-2-yl)-1H-pyrazol- | | |
| 4-yl)-1-(tetrahydro-2H- | | |
| pyran-2-yl)-1H-indazol-4- | | |
| amine 2.100 | | |
| 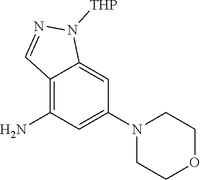 | tert-butyl ((2-chloro-[1,1′- biphenyl]-4-yl)methyl)(3- ((4-(6-morpholino-1- (tetrahydro-2H-pyran-2-yl)- 1H-indazol-4- yl)butyl)amino)-3- oxopropyl)carbamate 2.268 | LCMS (AM3): rt = 0.970 min, (731.5 [M + H]⁺), 91.4% purity Purification Method PM162 |
| 6-morpholino-1- | | |
| (tetrahydro-2H-pyran-2-yl)- | | |
| 1H-indazol-4-amine 2.267 | | |
| 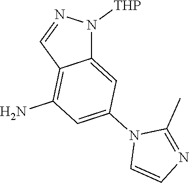 | tert-butyl ((2-chloro-[1,1′- biphenyl]-4-yl)methyl)(3- ((3-((6-(2-methyl-1H- imidazol-1-yl)-1- (tetrahydro-2H-pyran-2-yl)- 1H-indazol-4- yl)amino)propyl)amino)-3- oxopropyl)carbamate 2.271 | LCMS (AM3): rt = 0.950 min, (726.3 [M + H]⁺), 94.1% purity. Purification Method PM164 |
| 6-(2-methyl-1H-imidazol-1-yl]- | | |
| (tetrahydro-2H-pyran-2-yl)-1H- | | |
| indazol-4-amine 2.270 | | |

### Synthesis of Intermediate 1.225

### 3-((3-Chloro-4-(trifluoromethoxy)benzyl)amino)propanenitrile 1.223

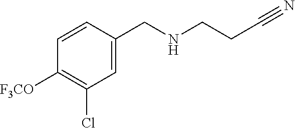 [SMILES: N#CCCNCc1ccc(OC(F)(F)F)c(Cl)c1]

A mixture of 3-chloro-4-(trifluoromethoxy)benzaldehyde (CAS 83279-39-4, 1 g, 4.45 mmol) and 3-aminopropanenitrile (780.32 mg, 11.13 mmol) in MeOH (20 mL) was stirred at 35° C. for 12 h, then NaBH(AcO)₃ (3.78 g, 17.81 mmol) was added. The reaction mixture was stirred at 35° C. for 3 h. The mixture was filtered and the filtrate was concentrated in vacuo. The residue was purified (PM61) to afford compound 1.223 (820 mg, 2.94 mmol, 66.1% yield) as a brown oil.

LCMS (AM3): rt=0.717 min, (278.9 [M+H]⁺), 100% purity.

### tert-Butyl 3-chloro-4-(trifluoromethoxy)benzyl(2-cyanoethyl)carbamate 1.224

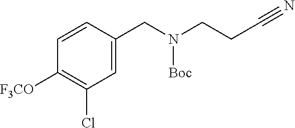 [SMILES: CC(C)(C)OC(=O)N(CCC#N)Cc1ccc(OC(F)(F)F)c(Cl)c1]

To a mixture of compound 1.223 (820 mg, 2.94 mmol) and TEA (893.31 mg, 8.83 mmol) in THF (10 mL) was added Boc₂O (706.45 mg, 3.24 mmol) at 20° C. The reaction mixture was stirred at 20° C. for 16 h. The mixture was concentrated in vacuo and the residue was purified (PM6) to afford compound 1.224 (1 g, 2.64 mmol, 89.7% yield) as a colourless oil.

LCMS (AM3): rt=1.207 min, (323.5 [M-tBu+2H]⁺), 91.3% purity.

### tert-Butyl 3-chloro-4-(trifluoromethoxy)benzyl(3-hydrazinyl-3-iminopropyl)carbamate 1.225

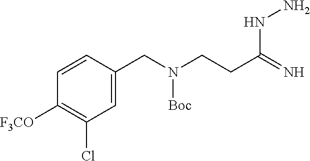 [SMILES: CC(C)(C)OC(=O)N(CCC(=N)NN)Cc1ccc(OC(F)(F)F)c(Cl)c1]

To a mixture of compound 1.224 (900 mg, 2.38 mmol) in a methanolic solution of NaOMe (2.45 M, 22.50 mL) was added hydrazine hydrate (1.55 g, 30.86 mmol) at RT. The reaction mixture was heated to 70° C. and stirred for 5 h. The mixture was adjusted to pH 7 by adding an aq. solution of FA (4 N). The mixture was filtered and the filtrate was concentrated in vacuo to give a residue that was purified (PM68) to afford compound 1.225 (170 mg, 413.81 μmol, 17.4% yield) as a colourless oil.

LCMS (AM3): rt=0.842 min, (411.1 [M+H]⁺), 79.8% purity.

### Synthesis of Intermediate 1.246

### 3-((3-Chloro-4-(trifluoromethoxy)benzyl)amino)propanoic acid 1.242

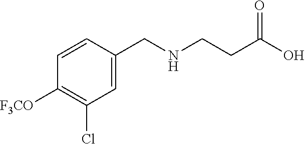 [SMILES: O=C(O)CCNCc1ccc(OC(F)(F)F)c(Cl)c1]

To a solution of 3-chloro-4-(trifluoromethoxy)benzaldehyde (CAS 83279-39-4, 7 g, 31.17 mmol) in MeOH (50 mL) was added 3-aminopropanoic acid (8.33 g, 93.51 mmol) at 20° C. After stirring for 24 h at 20° C., NaBH(AcO)₃ (19.82 g, 93.51 mmol) was added. The reaction mixture was stirred at 20° C. for 1 h. The mixture was filtered and the filtrate was concentrated in vacuo. The residue was purified (PM67) to afford compound 1.242 (7 g, 17.00 mmol, 54.5% yield, TFA salt) as a yellow oil.

LCMS (AM3): rt=0.703 min, (298.1 [M+H]⁺), 98.7% purity.

### 3-((tert-Butoxycarbonyl)(3-chloro-4-(trifluoromethoxy)benzyl)amino)propanoic acid 1.243

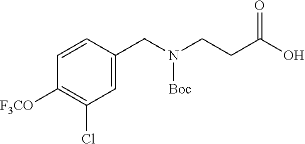 [SMILES: CC(C)(C)OC(=O)N(CCC(=O)O)Cc1ccc(OC(F)(F)F)c(Cl)c1]

To a solution of compound 1.242 (7 g, 23.52 mmol) in THF (50 mL) and H₂O (10 mL) was added NaHCO₃ (3.95 g, 47.03 mmol) and Boc₂O (7.70 g, 35.28 mmol) at 20° C. The reaction mixture was stirred at 20° C. for 12 h. The reaction mixture was acidified to pH 3 with aq. HCl (1 M) and then extracted with EA (50 mL×3). The combined organic layers were washed (brine, 20 mL), dried (Na₂SO₄), filtered and concentrated in vacuo. The residue was purified (PM2) to afford compound 1.243 (5.55 g, 13.95 mmol, 59.3% yield) as a yellow oil.

LCMS (AM3): rt=0.998 min, (420.0 [M+Na]⁺), 98.4% purity.

### Ethyl 4-(3-((tert-butoxycarbonyl)(3-chloro-4-(trifluoromethoxy)benzyl)amino)propanamido)-3-oxobutanoate 1.244

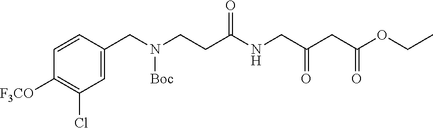 [SMILES: CCOC(=O)CC(=O)CNC(=O)CCN(Cc1ccc(OC(F)(F)F)c(Cl)c1)C(=O)OC(C)(C)C]

To a mixture of compound 1.243 (5.9 g, 14.83 mmol) and HATU (6.77 g, 17.80 mmol) in DMF (50 mL) was added DIPEA (5.75 g, 44.50 mmol) at 30° C. After stirring at 30° C. for 30 min, Ethyl 4-amino-3-oxobutanoate hydrochloride (2.69 g, 14.84 mmol, HCl salt) was added and the mixture was stirred at 30° C. for 2 h. The mixture was diluted with H₂O (100 mL) and the aq. phase was extracted with EA (100 mL×3). The combined organic phases were washed (brine, 200 mL), dried (Na₂SO₄), filtered and concentrated in vacuo. The residue was purified (PM61) to afford compound 1.244 (3.9 g, 7.43 mmol, 50.1% yield) as a red oil.

LCMS (AM3): rt=1.030 min, (525.1 [M+H]⁺), 74.0% purity.

### Ethyl 2-(2-(2-((tert-butoxycarbonyl)(3-chloro-4-(trifluoromethoxy)benzyl)amino)ethyl)oxazol-5-yl)acetate 1.245

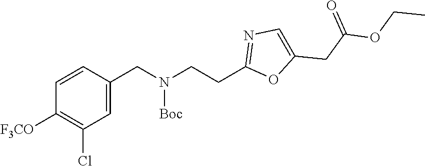 [SMILES: CCOC(=O)Cc1cnc(CCN(Cc2ccc(OC(F)(F)F)c(Cl)c2)C(=O)OC(C)(C)C)o1]

To a mixture of compound 1.244 (2.8 g, 5.33 mmol) in THF (40 mL) was added Burgess reagent (3.81 g, 16.00 mmol) at 20° C. The mixture was stirred at 20° C. for 2 h. The mixture was concentrated in vacuo and the residue was purified (PM4) to afford compound 1.245 (1.3 g, 2.56 mmol, 48% yield) as a yellow oil.

LCMS (AM3): rt=1.100 min, (507.2 [M+H]⁺), 94.0% purity.

### 2-(2-(2-((tert-Butoxycarbonyl)(3-chloro-4-(trifluoromethoxy)benzyl)amino)ethyl)oxazol-5-yl)acetic acid 1.246

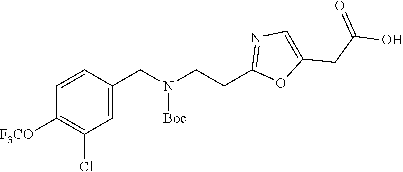 [SMILES: CC(C)(C)OC(=O)N(CCc1ncc(CC(=O)O)o1)Cc1ccc(OC(F)(F)F)c(Cl)c1]

To a mixture of compound 1.245 (1.1 g, 2.17 mmol) in THF (20 mL), MeOH (2 mL) and H₂O (2 mL) was added LiOH·H₂O (910.56 mg, 21.70 mmol) at 20° C. The mixture was stirred at 20° C. for 14 h. The mixture was acidified to pH 6 with aq. HCl (1 N) and the aq. phase was extracted with EA (30 mL×3). The combined organic phases were washed (brine, 80 mL), dried (Na₂SO₄), filtered and concentrated in vacuo to afford compound 1.246 (1.1 g) as a yellow oil, which was used without further purification.

LCMS (AM3): rt=1.002 min, (479.4 [M+H]⁺), 99.2% purity.

Synthesis of Intermediate 1.273 4-Nitro-1-(tetrahydro-2H-pyran-2-yl)-1H-indazol-6-amine 1.271

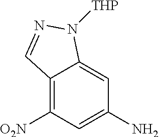

To a mixture of 6-bromo-4-nitro-1-(tetrahydro-2H-pyran-2-yl)-1H-indazole (WO2019027960, 2 g, 6.13 mmol) in DMSO (20 mL) was added ammonium hydroxide (2.27 g, 16.2 mmol), CuI (245 mg, 1.29 mmol), (2S, 4S)-4-hydroxypyrrolidine-2-carboxylic acid (330 mg, 2.51 mmol) and K₂CO₃ (2.54 g, 18.4 mmol) sequentially at rt. The mixture was heated to 90° C. and stirred for 16 h under N₂. The mixture was diluted with water (50 mL) and the aqueous phase was extracted with EA (50 mL×2). The combined organic phase was washed (brine, 90 mL×3), dried (Na₂SO₄), filtered and concentrated in vacuo to afford compound 1.271 (1.7 g) as a red oil.

LCMS (AM3): rt=0.769 min, (263.1 [M+H]⁺), 69.6% purity.

### 4-Nitro-1-(tetrahydro-2H-pyran-2-yl)-6-(4H-1,2,4-triazol-4-yl)-1H-indazole 1.272

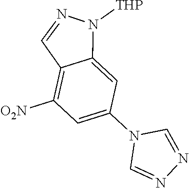

To a mixture of compound 1.271 (400 mg, 1.39 mmol), N-formamidoformamide (611 mg, 6.94 mmol) and TEA (983 mg, 9.72 mmol) in pyridine (8 mL) was added dropwise chlorotrimethylsilane (2.26 g, 20.8 mmol) at rt. The mixture was heated to 120° C. and stirred for 16 h. The mixture was concentrated in vacuo and the residue was diluted with water (30 mL). The aqueous phase was extracted with EA (30 mL×3) and the combined organic phases were washed (brine, 80 mL), dried (Na₂SO₄), filtered and concentrated in vacuo. The residue was purified (PM2) to afford compound 1.272 (100 mg, 0.318 mmol, 22.9% yield) as a yellow solid.

¹H NMR (400 MHz, CHCl₃-d) δ: 8.65 (s, 1H), 8.54 (s, 2H), 8.12 (d, J=2.0 Hz, 1H), 7.98 (d, J=0.8 Hz, 1H), 5.79 (dd, J=8.4, 2.8 Hz, 1H), 3.92-3.88 (m, 1H), 3.75-3.67 (m, 1H), 2.47-2.37 (m, 1H), 2.18-2.08 (m, 2H), 1.81-1.65 (m, 3H) ppm.

### 1-(Tetrahydro-2H-pyran-2-yl)-6-(4H-1,2,4-triazol-4-yl)-1H-indazol-4-amine 1.273

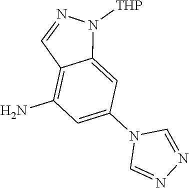

To a solution of compound 1.272 (100 mg, 0.318 mmol) in MeOH (10 mL) was added 10% Pd/C (30 mg) under N₂ at 25° C. The resulting suspension was degassed and purged with H₂ (×3). The mixture was stirred under a H₂ atmosphere (15 psi) at 25° C. for 2 h. The mixture was filtered and the filtrate concentrated in vacuo to afford compound 1.273 (90 mg) as a yellow solid.

LCMS (AM3): rt=0.640 min, (285.2 [M+H]⁺), 95.0% purity.

### Synthesis of Intermediate 1.280

### Benzyl (3-((3,3-diethoxypropyl)amino)-3-oxopropyl)carbamate 1.276

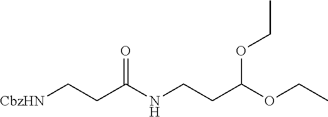 [SMILES: CCOC(CCNC(=O)CCNC(=O)OCc1ccccc1)OCC]

To a mixture of 3-(((benzyloxy)carbonyl)amino)propanoic acid (3 g, 13.44 mmol), HATU (6.13 g, 16.13 mmol), DIPEA (3.47 g, 26.88 mmol, 4.68 mL) in THF (50 mL) was added 3,3-diethoxypropan-1-amine (1.98 g, 13.44 mmol) at 20° C. The resulting mixture was stirred at 20° C. for 2 h. The reaction mixture was poured into water (50 mL) and extracted with EA (50 mL×3). The combined organic phases were washed (brine, 50 mL), dried (Na₂SO₄), filtered and concentrated in vacuo. The crude product was purified (PM2) to afford compound 1.276 (4.4 g, 12.48 mmol, 92.9% yield) as a yellow oil.

LCMS (AM3): rt=0.843 min, (375.2 [M+Na]⁺), 61.8% purity.

### Benzyl (3-oxo-3-((3-oxopropyl)amino)propyl)carbamate 1.277

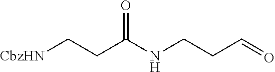 [SMILES: O=CCCNC(=O)CCNC(=O)OCc1ccccc1]

A mixture of compound 1.276 (2 g, 5.67 mmol) in water (8 mL) and AcOH (8 mL) was stirred at 20° C. for 1 h. The reaction mixture was diluted with water (50 mL) and basified with saturated aq. NaHCO₃ solution to pH=8. The mixture was extracted with EA (50 mL×3) and the combined organic phases were washed (brine, 50 mL), dried (Na₂SO₄), filtered and concentrated in vacuo to give compound 1.277 (2 g) as a white solid.

LCMS (AM3): rt=0.717 min, (279.2 [M+H]⁺), 93.6% purity.

### Benzyl (3-oxo-3-((3-((1-(tetrahydro-2H-pyran-2-yl)-6-(4H-1,2,4-triazol-4-yl)-1H-indazol-4-yl)amino)propyl)amino)propyl)carbamate 1.278

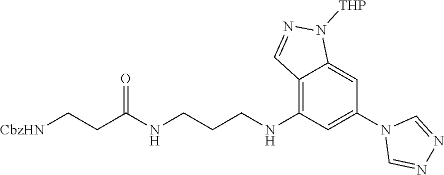

A mixture of compound 1.273 (680 mg, 2.39 mmol) and compound 1.277 (698.9 mg, 2.51 mmol) in DCE (10 mL) was stirred at 30° C. for 12 h, then NaBH(AcO)₃ (2.03 g, 9.57 mmol) was added. The mixture was stirred at 30° C. for 4 h. The mixture was concentrated in vacuo and the residue was purified (PM75) to afford compound 1.278 (500 mg, 0.915 mmol, 38.3% yield) as a yellow solid.

LCMS (AM3): rt=0.832 min, (547.4 [M+H]⁺), 95.7% purity.

### 3-Amino-N-(3-((1-(tetrahydro-2H-pyran-2-yl)-6-(4H-1,2,4-triazol-4-yl)-1H-indazol-4-yl)amino)propyl)propanamide 1.279

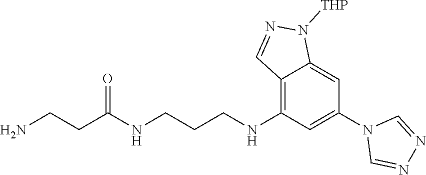

To a solution of compound 1.278 (350 mg, 0.640 mmol) in MeOH (30 mL) was added 10% Pd/C (100 mg) under N₂ at 20° C. The resulting suspension was degassed and purged with H₂ (×3). The mixture was stirred under a H₂ atmosphere (15 psi) at 20° C. for 16 h. The mixture was filtered and the filtrate was concentrated in vacuo to afford compound 1.279 (260 mg) as a white solid.

LCMS (AM3): rt=0.543 min, (413.2 [M+H]⁺), 87.8% purity.

### 3-((3-Chlorobenzyl)amino)-N-(3-((1-(tetrahydro-2H-pyran-2-yl)-6-(4H-1,2,4-triazol-4-yl)-1H-indazol-4-yl)amino)propyl)propanamide 1.280

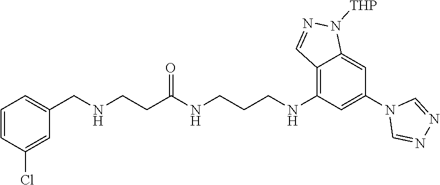

A mixture of 3-chlorobenzaldehyde (17.04 mg, 0.121 mmol) and compound 1.279 (50 mg, 0.121 mmol) in MeOH (1 mL) was stirred at 30° C. for 12 h, then NaBH(AcO)₃ (103 mg, 0.485 mmol) was added. The mixture was stirred at 30° C. for 14 h. The mixture was concentrated in vacuo and the residue was purified (PM76) to afford compound 1.280 (20 mg, 0.03724 mmol, 30.7% yield) as a white solid.

LCMS (AM3): rt=0.782 min, (537.2 [M+H]⁺), 100% purity.

### Synthesis of Intermediate 1.283

### tert-Butyl methyl(3-((1-(tetrahydro-2H-pyran-2-yl)-6-(4H-1,2,4-triazol-4-yl)-1H-indazol-4-yl)amino)propyl)carbamate 1.281

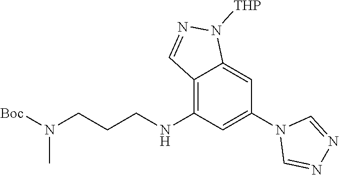

A solution of compound 1.273 (150 mg, 527.58 μmol) and tert-butyl methyl(3-oxopropyl)carbamate (98.78 mg, 527.58 μmol) in DCE (6 mL) was stirred at 30° C. for 12 h, then NaBH(AcO)₃ (223.63 mg, 1.06 mmol) was added. The mixture was stirred at 30° C. for 1 h. The mixture was concentrated in vacuo and the crude product was purified (PM67) to afford compound 1.281 (61 mg, 131.76 μmol, 25% yield) as a yellow solid.

LCMS (AM3): rt=0.868 min, (456.2 [M+H]⁺), 98.5% purity.

### N¹-(6-(4H-1,2,4-triazol-4-yl)-1H-indazol-4-yl)-N³-methylpropane-1,3-diamine 1.282

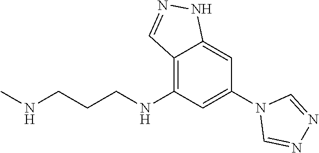 [SMILES: CNCCCNc1cc(-n2cnnc2)cc2[nH]ncc12]

A solution of compound 1.281 (125 mg, 274.39 μmol) in aq. HCl (6 M, 5 mL) was stirred at 20° C. for 0.5 h. The mixture was concentrated in vacuo to afford compound 1.282 (105 mg, HCl salt) as a yellow oil.

LCMS (AM3): rt=0.297 min, (272.2 [M+H]⁺), 93.6% purity.

### tert-Butyl (3-((3-((6-(4H-1,2,4-triazol-4-yl)-1H-indazol-4-yl)amino)propyl)(methyl)amino)-3-oxopropyl)(3-chloro-4-(trifluoromethoxy)benzyl)carbamate 1.283

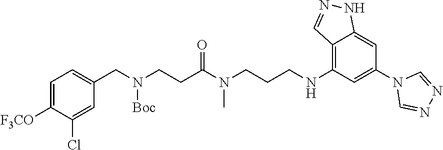 [SMILES: CN(CCCNc1cc(-n2cnnc2)cc2[nH]ncc12)C(=O)CCN(Cc1ccc(OC(F)(F)F)c(Cl)c1)C(=O)OC(C)(C)C]

To a solution of compound 1.243 (109.85 mg, 276.17 μmol) in DMF (2 mL) was added HATU (126.01 mg, 331.40 μmol) and DIPEA (192.42 μL, 1.10 mmol) at 20° C. After stirring at 20° C. for 0.5 h, compound 1.282 (85 mg, 276.17 μmol) was added and the reaction mixture was stirred at 20° C. for 1.5 h. The mixture was poured into water (50 mL) and extracted with EA (50 mL×2). The combined organic phases were washed (brine, 50 mL), dried (Na₂SO₄) and concentrated in vacuo. The crude product was purified (PM67) to afford compound 1.283 (70 mg, 107.5 μmol, 31.7% yield) as a red solid.

LCMS (AM3): rt=0.965 min, (651.2 [M+H]⁺), 41.1% purity.

### Synthesis of Intermediate 1.286

### tert-Butyl (3-oxo-3-((1-(tetrahydro-2H-pyran-2-yl)-6-(4H-1,2,4-triazol-4-yl)-1H-indazol-4-yl)amino)propyl)carbamate 1.284

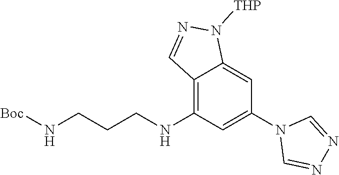

A mixture of compound 1.273 (200 mg, 0.703 mmol), 3-((tert-butoxycarbonyl)amino)propanoic acid (133 mg, 0.703 mmol) and EDCI (404 mg, 2.11 mmol) in pyridine (4 mL) was stirred at 80° C. for 4 h. The reaction mixture was concentrated in vacuo and the residue was purified (PM67) to afford compound 1.284 (57 mg, 17.8% yield) as a white solid.

LCMS (AM3): rt=0.841 min, (456.2 [M+H]⁺), 98.0% purity.

### N-(6-(4H-1,2,4-triazol-4-yl)-1H-indazol-4-yl)-3-aminopropanamide 1.285

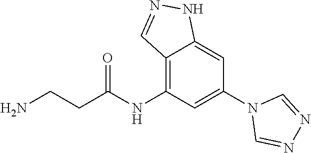 [SMILES: NCCC(=O)Nc1cc(-n2cnnc2)cc2[nH]ncc12]

A mixture of compound 1.284 (100 mg, 0.22 mmol) in a solution of HCl in 1,4-dioxane (4 M, 10 mL) was stirred at RT for 1 h, white solid precipitated. The reaction mixture was filtered and the filter cake was dried in vacuo to afford compound 1.285 (72 mg, HCl salt) as a white solid.

LCMS (AM3): rt=0.126 min, (272.1 [M+H]⁺), 98.4% purity.

### tert-Butyl (3-((3-((6-(4H-1,2,4-triazol-4-yl)-1H-indazol-4-yl)amino)-3-oxopropyl)amino)-3-oxopropyl)(3-chloro-4-(trifluoromethoxy)benzyl)carbamate 1.286

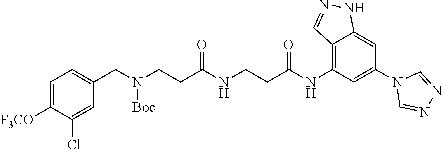 [SMILES: CC(C)(C)OC(=O)N(CCC(=O)NCCC(=O)Nc1cc(-n2cnnc2)cc2[nH]ncc12)Cc1ccc(OC(F)(F)F)c(Cl)c1]

To a solution of compound 1.243 (100 mg, 0.251 mmol) in DMF (5 mL) was added HATU (107 mg, 0.281 mmol) and DIPEA (0.122 mL, 0.7 mmol) followed by compound 1.285 (72 mg, 0.234 mmol, HCl salt), the reaction mixture was stirred at rt for 20 h. The reaction mixture was poured into water (30 mL) and the resulting mixture was extracted with EA (10 mL×3). The combined organic phases were washed (brine, 30 mL), dried (Na₂SO₄), filtered and concentrated in vacuo. The residue was purified (PM80) to afford compound 1.286 (35 mg, 23% yield) as a white solid.

LCMS (AM3): rt=0.923 min, (651.2 [M+H]⁺), 100% purity.

### Synthesis of Intermediate 1.289

### tert-Butyl (2-oxo-2-((1-(tetrahydro-2H-pyran-2-yl)-6-(4H-1,2,4-triazol-4-yl)-1H-indazol-4-yl)amino)ethyl)carbamate 1.287

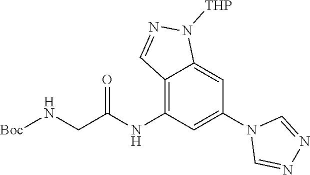

A mixture of compound 1.273 (200 mg, 0.703 mmol), 2-((tert-butoxycarbonyl)amino)acetic acid (135 mg, 0.771 mmol) and EDCI (400 mg, 2.09 mmol) in pyridine (4 mL) was stirred 85° C. for 18 h. The reaction mixture was concentrated in vacuo and the residue was purified (PM67) to afford compound 1.287 (104 mg, 32.1% yield) as a yellow solid.

LCMS (AM3): rt=0.828 min, (442.3 [M+H]⁺), 96.6% purity.

### N-(6-(4H-1,2,4-triazol-4-yl)-1H-indazol-4-yl)-2-aminoacetamide 1.288

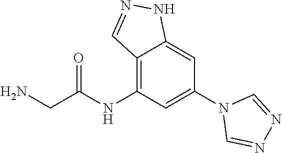 [SMILES: NCC(=O)Nc1cc(-n2cnnc2)cc2[nH]ncc12]

A mixture of compound 1.287 (104 mg, 0.236 mmol) in a solution of HCl in 1,4-dioxane (4 M, 10 mL) was stirred at RT for 14 h, white solid precipitated. The reaction mixture was filtered and the filter cake was dried in vacuo to afford compound 1.288 (62 mg, HCl salt) as a white solid, which was used directly without further purification.

### tert-Butyl (4-((2-((6-(4H-1,2,4-triazol-4-yl)-1H-indazol-4-yl)amino)-2-oxoethyl)amino)butyl)(3-chloro-4-(trifluoromethoxy)benzyl)carbamate 1.289

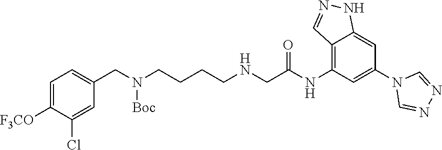 [SMILES: CC(C)(C)OC(=O)N(CCCCNCC(=O)Nc1cc(-n2cnnc2)cc2[nH]ncc12)Cc1ccc(OC(F)(F)F)c(Cl)c1]

A mixture of compound 1.288 (62 mg, 0.211 mmol, HCl salt), Intermediate 1 (90 mg, 0.227 mmol) and NaOAc (35 mg, 0.427 mmol) in MeOH (10 mL) was stirred at rt for 12 h, then NaBH₃CN (53 mg, 0.843 mmol) was added. The reaction mixture was stirred at rt for 2 h. The reaction mixture was concentrated in vacuo and the residue was purified (PM81) to afford compound 1.289 (50 mg, 37.1% yield) as a white solid.

LCMS (AM3): rt=0.879 min, (637.2 [M+H]⁺), 100% purity.

### Synthesis of Intermediate 1.292

### tert-Butyl (2-((1-(tetrahydro-2H-pyran-2-yl)-6-(4H-1,2,4-triazol-4-yl)-1H-indazol-4-yl)amino)ethyl)carbamate 1.290

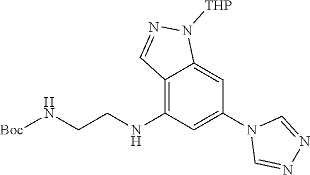

To a solution of compound 1.287 (450 mg, 1.02 mmol) in THF (20 mL) was added borane tetrahydrofuran complex (1 M, 10 mL) at 0° C. The reaction mixture was then warmed to rt and stirred for 20 h. The reaction was quenched by addition of aq. NaOH solution (1 N, 20 mL) slowly at 0° C., then the mixture was heated to 60° C. and stirred for 3 h. The reaction mixture was extracted with EA (20 mL×3) and the combined organic phases were washed (brine, 60 mL), dried (Na₂SO₄), filtered and concentrated in vacuo. The residue was purified (PM82) to afford compound 1.290 (120 mg, 27.2% yield) as a brown solid.

LCMS (AM3): rt=0.847 min, (428.2 [M+H]⁺), 98.9% purity.

### N¹-(6-(4H-1,2,4-triazol-4-yl)-1H-indazol-4-yl)ethane-1,2-diamine 1.291

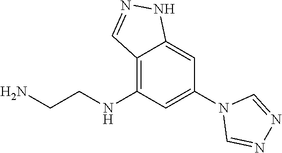 [SMILES: NCCNc1cc(-n2cnnc2)cc2[nH]ncc12]

A mixture of compound 1.290 (120 mg, 0.281 mmol) in a solution of HCl in MeOH (4 M, 5 mL) was stirred at RT for 1 h. The reaction mixture was concentrated in vacuo to afford compound 1.291 (76 mg, HCl salt) as a brown solid.

LCMS (AM3): rt=0.134 min, (244.2 [M+H]⁺), 100% purity.

### tert-Butyl (4-((2-((6-(4H-1,2,4-triazol-4-yl)-1H-indazol-4-yl)amino)ethyl)amino)butyl)(3-chloro-4-(trifluoromethoxy)benzyl)carbamate 1.292

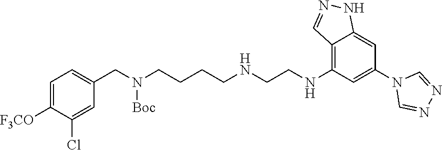 [SMILES: CC(C)(C)OC(=O)N(CCCCNCCNc1cc(-n2cnnc2)cc2[nH]ncc12)Cc1ccc(OC(F)(F)F)c(Cl)c1]

To a mixture of compound 1.291 (66 mg, 0.236 mmol, HCl salt), DIPEA (0.488 mmol, 0.085 mL) and Intermediate I (100 mg, 0.253 mmol) in MeOH (2 mL) was added NaBH₃CN (60 mg, 0.955 mmol) at RT. The reaction mixture was stirred at rt for 4 h. The reaction mixture was concentrated in vacuo and the residue purified (PM83) to afford compound 1.292 (80 mg, 46% yield, TFA salt) as a white solid.

LCMS (AM3): rt=0.860 min, (623.2 [M+H]⁺), 99.5% purity.

### Synthesis of Intermediate 1.294

### 4-Nitro-1-(tetrahydro-2H-pyran-2-yl)-6-(1H-1,2,4-triazol-1-yl)-1H-indazole 1.293

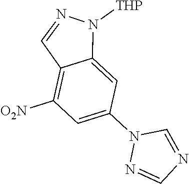

To a mixture of 6-bromo-4-nitro-1-(tetrahydro-2H-pyran-2-yl)-1H-indazole (WO2019027960, 1.0 g, 3.06 mmol) and 1,2,4-triazole (423.5 mg, 6.15 mmol) in DMF (25 mL) was added CuI (58.4 mg, 0.305 mmol), Cs₂CO₃ (2.0 g, 6.15 mmol) and (1R,2R)-N¹,N₂-dimethylcyclohexane-1,2-diamine (87.2 mg, 0.615 mmol) at rt. The mixture was heated to 110° C. and stirred for 16 h under N₂. The mixture was diluted with water (30 mL) and the aqueous phase was extracted with EA (30 mL×2). The combined organic phases were washed (brine, 50 mL×3), dried (Na₂SO₄), filtered and concentrated in vacuo. The residue was purified (PM3) to afford compound 1.293 (160 mg, 0.509 mmol, 16.6% yield) as a yellow solid.

¹H NMR (400 MHz, CHCl₃-d) δ: 8.76 (s, 1H), 8.70 (d, J=0.6 Hz, 1H), 8.50 (d, J=1.6 Hz, 1H), 8.39 (d, J=0.8 Hz, 1H), 8.21 (s, 1H), 5.89 (dd, J=8.4, 2.4 Hz, 1H), 4.03-3.96 (m, 1H), 3.84-3.77 (m, 1H), 2.60-2.52 (m, 1H), 2.24-2.16 (m, 2H), 1.88-1.72 (m, 3H) ppm.

### 1-(Tetrahydro-2H-pyran-2-yl)-6-(1H-1,2,4-triazol-1-yl)-1H-indazol-4-amine 1.294

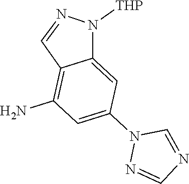

To a solution of compound 1.293 (160 mg, 0.509 mmol) in MeOH (10 mL) was added 10% Pd/C (50 mg) under N₂ at 25° C. The resulting suspension was degassed and purged with H₂ (×3). The mixture was stirred under H₂ (15 psi) at 25° C. for 4 h. The mixture was filtered and the filtrate was concentrated in vacuo to afford compound 1.294 (140 mg) as a white solid.

LCMS (AM3): rt=0.686 min, (285.2 [M+H]⁺), 93.7% purity.

### Synthesis of Intermediate 1.296

### tert-Butyl 3-chloro-4-(trifluoromethoxy)benzyl(2-(5-(2-oxo-2-((1-(tetrahydro-2H-pyran-2-yl)-6-(4H-1,2,4-triazol-4-yl)-1H-indazol-4-yl)amino)ethyl)oxazol-2-yl)ethyl)carbamate 1.296

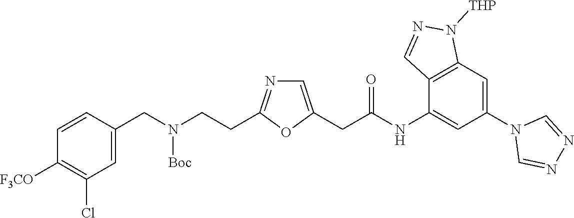

To a mixture of compound 1.273 (100 mg, 351.72 μmol), compound 1.246 (100 mg, 208.84 μmol) and DIPEA (107.96 mg, 835.34 μmol) in THF (2 mL) was added T₃P (50% in EA) (265.79 mg, 417.67 μmol) at 20° C. The mixture was stirred at 20° C. for 12 h. The mixture was diluted with water (30 mL) and the aq. phase was extracted with EA (30 mL×3). The combined organic phases were washed (brine, 90 mL), dried (Na₂SO₄), filtered and concentrated in vacuo. The residue was purified (PM86) to afford compound 1.296 (80 mg, 107.36 μmol, 51.4% yield) as a white solid.

LCMS (AM3): rt=0.991 min, (745.2 [M+H]⁺), 29.1% purity.

### Synthesis of Intermediate 1.300

### Methyl 2-(5-(2-((tert-butoxycarbonyl)(3-chloro-4-(trifluoromethoxy)benzyl)amino)ethyl)-4H-1,2,4-triazol-3-yl)acetate 1.297

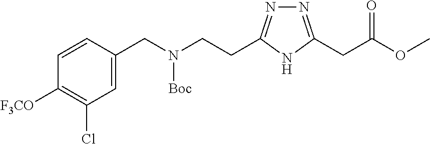 [SMILES: COC(=O)Cc1nnc(CCN(Cc2ccc(OC(F)(F)F)c(Cl)c2)C(=O)OC(C)(C)C)[nH]1]

To a mixture of compound 1.225 (950 mg, 2.31 mmol) and TEA (701.99 mg, 6.94 mmol) in DMF (10 mL) was added methyl 3-chloro-3-oxo-propanoate (405.33 mg, 2.97 mmol) at 0° C. The mixture was warmed to 20° C. and stirred for 1 h, then heated to 110° C. and stirred for 2 h. The mixture was diluted with water (30 mL) and the aq. phase was extracted with EA (30 mL×3). The combined organic phases were washed (brine, 90 mL), dried (Na₂SO₄), filtered and concentrated in vacuo. The residue was purified (PM67) to afford compound 1.297 (400 mg, 811.56 μmol, 35.1% yield) as a yellow oil.

LCMS (AM3): rt=0.965 min, (493.4 [M+H]⁺), 86.3% purity.

### 2-(5-(2-((tert-Butoxycarbonyl)(3-chloro-4-(trifluoromethoxy)benzyl)amino)ethyl)-4H-1,2,4-triazol-3-yl)acetic acid 1.298

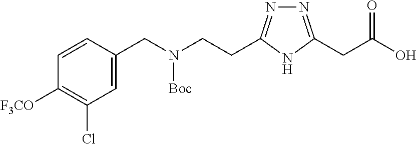 [SMILES: CC(C)(C)OC(=O)N(CCc1nnc(CC(=O)O)[nH]1)Cc1ccc(OC(F)(F)F)c(Cl)c1]

To a mixture of compound 1.297 (560 mg, 1.14 mmol) in THF (10 mL), MeOH (2 mL) and water (2 mL) was added lithium hydroxide monohydrate (476.79 mg, 11.36 mmol) at 20° C. The mixture was stirred at 20° C. for 12 h. The mixture was concentrated in vacuo to remove the organic solvent and then the aqueous phase was acidified with aq. HCl (1 M) to pH=4. The mixture was extracted with EA (30 mL×3) and the combined organic phases were washed (brine, 80 mL), dried (Na₂SO₄), filtered and concentrated in vacuo to afford compound 1.298 (540 mg) as a yellow oil.

LCMS (AM3): rt=0.920 min, (479.4 [M+H]⁺), 91.0% purity.

### tert-Butyl 3-chloro-4-(trifluoromethoxy)benzyl(2-(5-(2-oxo-2-((1-(tetrahydro-2H-pyran-2-yl)-6-(4H-1,2,4-triazol-4-yl)-1H-indazol-4-yl)amino)ethyl)-4H-1,2,4-triazol-3-yl)ethyl)carbamate 1.299

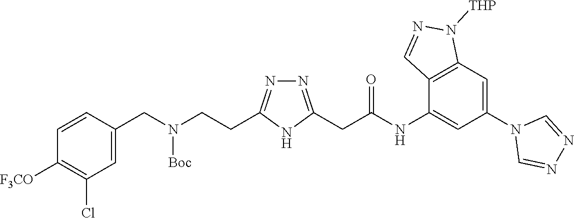

To a mixture of compound 1.298 (240 mg, 501.20 μmol), compound 1.273 (185.25 mg, 651.56 μmol) and DIPEA (259.11 mg, 2.00 mmol) in THF (5 mL) was added T₃P (50% in EA) (856.00 mg, 1.35 mmol) at 30° C. The mixture was stirred at 30° C. for 3 h. The mixture was diluted with water (30 mL) and the aqueous phase was extracted with EA (30 mL×3). The combined organic phases were washed (brine, 90 mL), dried (Na₂SO₄), filtered and concentrated in vacuo. The residue was purified (PM92) to afford compound 1.299 (170 mg, 228.14 μmol, 45.5% yield) as a white solid.

LCMS (AM3): rt=0.985 min, (745.2 [M+H]⁺), 94.3% purity.

### tert-Butyl 3-chloro-4-(trifluoromethoxy)benzyl(2-(5-(2-((1-(tetrahydro-2H-pyran-2-yl)-6-(4H-1,2,4-triazol-4-yl)-1H-indazol-4-yl)amino)ethyl)-4H-1,2,4-triazol-3-yl)ethyl)carbamate 1.300

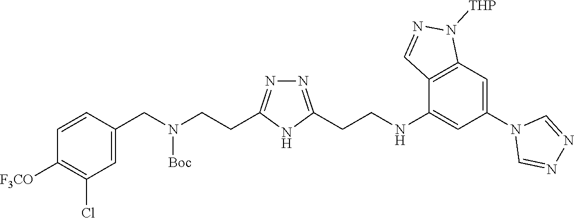

To a mixture of compound 1.299 (170 mg, 228.14 μmol) in 2-methyltetrahydrofuran (5 mL) was added borane tetrahydrofuran complex (1 M, 2.28 mL) at 0° C. The mixture was warmed to 20° C. and stirred for 4 h. The mixture was quenched by addition of MeOH (5 mL) and aq. NaOH (1 N, 5 mL) at 0° C., and then heated to 60° C. and stirred for 12 h. The aqueous phase was extracted with EA (30 mL×2) and the combined organic phases were washed (brine, 50 mL), dried (Na₂SO₄), filtered and concentrated in vacuo. The residue was purified (PM92) to afford compound 1.300 (50 mg, 68.38 μmol, 29.9% yield) as a brown solid.

LCMS (AM3): rt=0.969 min, (731.2 [M+H]⁺), 96.1% purity.

### Synthesis of Intermediate 1.302

### 4-Nitro-6-(1H-pyrazol-4-yl)-1-(tetrahydro-2H-pyran-2-yl)-1H-indazole 1.301

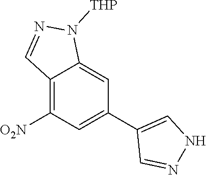

To a mixture of 6-bromo-4-nitro-1-(tetrahydro-2H-pyran-2-yl)-1H-indazole (WO2019027960, 670 mg, 2.05 mmol), Cs₂CO₃ (1.34 g, 4.11 mmol) and tert-butyl 4-(4,4,5,5-tetramethyl-1,3,2-dioxaborolan-2-yl)-1H-pyrazole-1-carboxylate (725 mg, 2.47 mmol) in 1,4-dioxane (10 mL) and water (1 mL) was added Pd(dppf)Cl₂ (150 mg, 0.205 mmol) at RT. The mixture was heated to 90° C. and stirred for 16 h under N₂. The mixture was concentrated in vacuo and the residue was purified (PM3) to afford compound 1.301 (370 mg, 1.18 mmol, 57.5% yield) as a yellow solid.

LCMS (AM3): rt=0.788 min, (314.1 [M+H]⁺), 42.1% purity.

### 6-(1H-pyrazol-4-yl)-1-(tetrahydro-2H-pyran-2-yl)-1H-indazol-4-amine 1.302

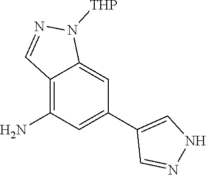

To a solution of compound 1.301 (270 mg, 0.862 mmol) in MeOH (10 mL) was added 10% Pd/C (50 mg) under N₂ protection at 25° C. The resulting suspension was degassed and purged with H₂ (×3). The mixture was stirred under a hydrogen atmosphere (15 psi) at 25° C. for 50 min. The mixture was filtered and the filtrate was concentrated in vacuo to afford compound 1.302 (240 mg) as a yellow solid.

LCMS (AM3): rt=0.628 min, (306.1 [M+Na]⁺), 47.0% purity.

### Synthesis of Intermediate 1.306

### tert-Butyl (4-(2-oxo-2-((1-(tetrahydro-2H-pyran-2-yl)-6-(4H-1,2,4-triazol-4-yl)-1H-indazol-4-yl)amino)ethoxy)butyl)carbamate 1.304

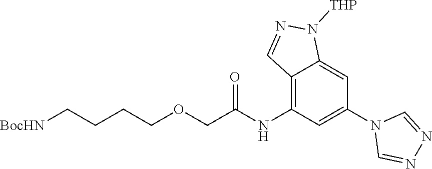

A mixture of 2-(3-((tert-butoxycarbonyl)amino)propoxy)acetic acid (US2015297738, 2 g, 8.09 mmol), compound 1.273 (2 g, 7.03 mmol) and EDCI (4.05 g, 21.11 mmol) in pyridine (20 mL) was stirred at 80° C. for 12 h. The reaction mixture was concentrated in vacuo and the residue was purified (PM67) to afford compound 1.304 (1.3 g, 36% yield) as a brown solid.

LCMS (AM3): rt=0.875 min, (514.5 [M+H]⁺), 100% purity.

### tert-Butyl (4-(2-((1-(tetrahydro-2H-pyran-2-yl)-6-(4H-1,2,4-triazol-4-yl)-1H-indazol-4-yl)amino)ethoxy)butyl)carbamate 1.305

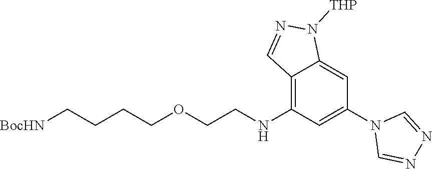

To a solution of compound 1.304 (1.3 g, 2.53 mmol) in THF (25 mL) was added borane tetrahydrofuran complex (1 M, 25 mL) under N₂ at rt. The reaction mixture was stirred at RT for 3 h. The reaction mixture was quenched by slow addition of aq. NaOH solution (1 N, 25 mL), then the resulting mixture was heated to 60° C. and stirred for 4 h. Water (25 mL) was added to the reaction mixture and the mixture was extracted with EA (25 mL×3). The combined organic phases were washed (brine, 50 mL), dried (Na₂SO₄), filtered and concentrated in vacuo. The residue was purified (PM95) to afford compound 1.305 (360 mg, 28.5% yield) as a brown solid.

LCMS (AM3): rt=0.902 min, (500.5 [M+H]⁺), 93.1% purity.

### N-(2-(4-aminobutoxy)ethyl)-6-(4H-1,2,4-triazol-4-yl)-1H-indazol-4-amine 1.306

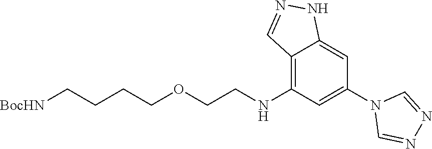 [SMILES: CC(C)(C)OC(=O)NCCCCOCCNc1cc(-n2cnnc2)cc2[nH]ncc12]

A mixture of compound 1.305 (360 mg, 0.721 mmol) in a solution of HCl in 1,4-dioxane (4 M, 10 mL) was stirred at RT for 16 h. The reaction mixture was concentrated in vacuo to afford compound 1.306 (270 mg, HCl salt) as a brown solid, which was used directly without further purification.

### Synthesis of Intermediate 1.307

### N-(6-(4H-1,2,4-triazol-4-yl)-1H-indazol-4-yl)-2-(4-aminobutoxy)acetamide 1.307

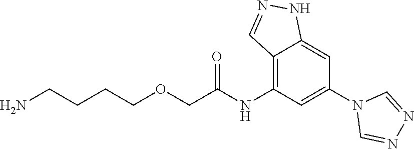 [SMILES: NCCCCOCC(=O)Nc1cc(-n2cnnc2)cc2[nH]ncc12]

A mixture of compound 1.304 (100 mg, 0.195 mmol) in a solution of HCl in 1,4-dioxane (4 M, 10 mL) was stirred at RT for 15 h, yellow solid precipitated. The reaction mixture was filtered and the filter cake was dried in vacuo to afford compound 1.307 (64 mg, 89.9% yield, HCl salt) as a yellow solid.

LCMS (AM3): rt=0.167 min, (330.3 [M+H]⁺), 97.1% purity.

### Synthesis of Intermediate 1.311

### Methyl 1-methyl-5-(trifluoromethyl)-1H-indole-2-carboxylate 1.309

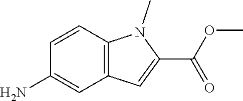

To a mixture of methyl 5-(trifluoromethyl)-1H-indole-2-carboxylate (CAS 1362860-89-6, 1.5 g, 6.17 mmol) in DMF (10 mL) was added NaH (296.08 mg, 7.40 mmol, 60% dispersion in oil) in one portion at 0° C. under N₂. After being stirred for 0.5 h, iodomethane (1.75 g, 12.34 mmol) was added. The mixture was warmed to 25° C. and stirred for 11.5 h. The mixture was poured into H₂O (50 mL) and the aq. phase was extracted with EA (30 mL×2). The combined organic phase was washed (brine, 30 mL), dried (Na₂SO₄), filtered and concentrated in vacuo. The residue was purified (PM6) to afford compound 1.309 (1.2 g, 4.67 mmol, 75.6% yield) as a white solid.

¹H NMR (400 MHz, MeOH-d₄) δ: 7.98-7.96 (s, 1H), 7.63-7.58 (m, 1H), 7.56-7.52 (m, 1H), 7.34 (s, 1H), 4.06 (s, 3H), 3.90 (s, 3H) ppm.

### (1-Methyl-5-(trifluoromethyl)-1H-indol-2-yl)methanol 1.310

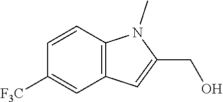 [SMILES: Cn1c(CO)cc2cc(C(F)(F)F)ccc21]

To a mixture of compound 1.309 (1.2 g, 4.67 mmol) in THF (10 mL) was added LAH (177.06 mg, 4.67 mmol) in one portion under N₂ at 25° C. The mixture was stirred at 25° C. for 0.5 h. The mixture was cooled to 0° C. and diluted with EA (10 mL). The mixture was quenched by addition of H₂O (0.2 mL), 10% NaOH aq. (0.2 mL) and H₂O (0.6 mL), sequentially. The resulting suspension was dried (Na₂SO₄), filtered and concentrated in vacuo to afford compound 1.310 (1 g) as a white solid, which was used without further purification.

### 1-Methyl-5-(trifluoromethyl)-1H-indole-2-carbaldehyde 1.311

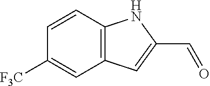 [SMILES: O=Cc1cc2cc(C(F)(F)F)ccc2[nH]1]

To a solution of compound 1.310 (1 g, 4.36 mmol) in DCM (10 mL) was added manganese (IV) oxide (758.65 mg, 8.73 mmol) at 25° C. The mixture was stirred at 25° C. for 2 h. The mixture was concentrated in vacuo and the residue was purified (PM6) to afford compound 1.311 (300 mg, 1.32 mmol, 30.3% yield) as a yellow solid.

¹H NMR (400 MHz, MeOH-d₄) δ: 9.92 (s, 1H), 8.09 (s, 1H), 7.70-7.60 (q, 2H), 7.48 (s, 1H), 4.10 (s, 3H) ppm.

### Synthesis of Intermediate 1.320

### N-(2-chloroethyl)-1-(tetrahydro-2H-pyran-2-yl)-6-(4H-1,2,4-triazol-4-yl)-1H-indazol-4-amine 1.318

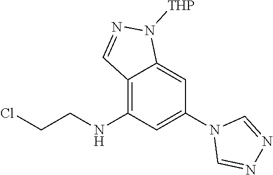

To a solution of 2-chloroacetaldehyde (4.33 mL, 26.91 mmol, 40% aqueous solution) and compound 1.273 (500 mg, 1.76 mmol) in MeOH (15 mL) was added NaBH₃CN (1.11 g, 17.59 mmol) at 20° C. The reaction mixture was stirred at 20° C. for 2.5 h. The reaction mixture was filtered and concentrated in vacuo and the crude product was purified (PM96) to give compound 1.318 (85 mg, 245.09 μmol, 13.9% yield) as a brown solid.

LCMS (AM3): rt=0.824 min, (347.1 [M+H]⁺), 95.5% purity.

### N-(2-azidoethyl)-1-(tetrahydro-2H-pyran-2-yl)-6-(4H-1,2,4-triazol-4-yl)-1H-indazol-4-amine 1.319

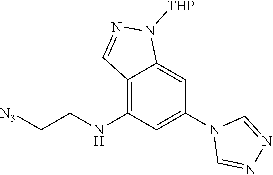

To a solution of compound 1.318 (85 mg, 245.09 μmol) in DMF (15 mL) was added sodium azide (0.140 g, 2.15 mmol) slowly at rt. The reaction mixture was heated to 50° C. and stirred for 12 h. The reaction mixture was diluted with H₂O (50 mL) and extracted with EA (50 mL×3). The combined organic layer was washed (brine, 50 mL×5), dried (Na₂SO₄), filtered and concentrated in vacuo to afford compound 1.319 (100 mg) as a yellow solid.

LCMS (AM3): rt=0.824 min, (354.4[M+H]⁺), 97.1% purity.

### tert-Butyl ((2-chloro-[1,1′-biphenyl]-4-yl)methyl)(2-(1-(2-((1-(tetrahydro-2H-pyran-2-yl)-6-(4H-1,2,4-triazol-4-yl)-1H-indazol-4-yl)amino)ethyl)-1H-1,2,3-triazol-4-yl)ethyl)carbamate 1.320

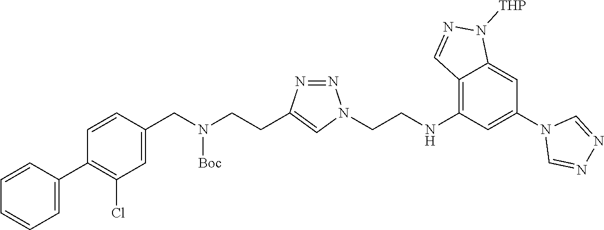

To a solution of compound 1.319 (100 mg, 282.98 μmol) and Intermediate J (104.67 mg, 282.98 μmol) in DMF (7 mL) and MeOH (1 mL) was added CuI (10.78 mg, 56.60 μmol) at rt. The reaction mixture was heated to 100° C. and stirred for 16 h under N₂. The reaction mixture was diluted with water (30 mL) and extracted with EA (50 mL×3). The combined organic layer was washed (brine, 80 mL×4), dried (Na₂SO₄), filtered and concentrated in vacuo to afford compound 1.320 (240 mg) as a brown oil.

LCMS (AM3): rt=1.030 min, (723.3[M+H]⁺), 56.6% purity.

### Synthesis of Intermediate 1.322

### 4-Nitro-6-(pyridin-4-yl)-1-(tetrahydro-2H-pyran-2-yl)-1H-indazole 1.321

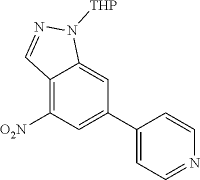

To a mixture of 6-bromo-4-nitro-1-(tetrahydro-2H-pyran-2-yl)-1H-indazole (WO2019027960, 2.5 g, 7.67 mmol), pyridin-4-yl boronic acid (1.88 g, 15.33 mmol) and K₂CO₃
(2.12 g, 15.33 mmol) in 1,4-dioxane (20 mL) and water (2 mL) was added Pd(dppf)Cl₂·CH₂Cl₂ (625.98 mg, 766.53 μmol) under N₂ at RT. The mixture was heated to 80° C. and stirred for 12 h. The reaction mixture was concentrated in vacuo and the residue purified (PM2) to afford compound 1.321 (2 g, 5.86 mmol, 76.4% yield) as a yellow gum.

LCMS (AM3): rt=0.789 min, (325.2 [M+H]⁺), 95.4% purity.

### 6-(Pyridin-4-yl)-1-(tetrahydro-2H-pyran-2-yl)-1H-indazol-4-amine 1.322

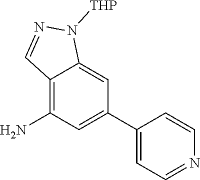

To a solution of compound 1.321 (2.0 g, 6.17 mmol) in MeOH (100 mL) was added 10% Pd/C (100 mg, 6.17 mmol) under N₂ protection at 25° C. The suspension was degassed and purged with H₂ (×3). The mixture was stirred under a H₂ atmosphere (15 psi) at 25° C. for 2 h. The reaction mixture was filtered, the filtrate was concentrated in vacuo to afford compound 1.322 (1.7 g) as a yellow gum.

LCMS (AM3): rt=0.568 min, (295.2 [M+H]⁺), 95.0% purity.

### Synthesis of Intermediate 1.326

### tert-Butyl 3-chloro-4-(trifluoromethoxy)benzyl(3-((3,3-diethoxypropyl)amino)-3-oxopropyl)carbamate 1.324

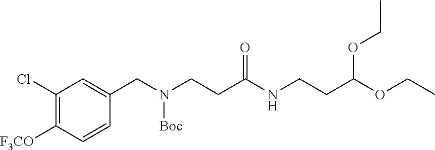 [SMILES: CCOC(CCNC(=O)CCN(Cc1ccc(OC(F)(F)F)c(Cl)c1)C(=O)OC(C)(C)C)OCC]

To a solution of compound 1.243 (1.3 g, 3.27 mmol), 3,3-diethoxypropan-1-amine (577.35 mg, 3.92 mmol) and TEA (1.4 mL, 10.06 mmol) in THF (30 mL) was added T₃P (50% in EA) (6.73 mmol, 4 mL) at 20° C. The resulting mixture was stirred at 20° C. for 1 h. The residue was poured onto water (100 mL) and extracted with EA (50 mL×3). The combined organic phases were washed (brine, 50 mL×3), dried (Na₂SO₄), filtered and concentrated in vacuo. The residue was purified (PM2) to give compound 1.324 (933 mg, 1.77 mmol, 54.1% yield) as a yellow oil.

LCMS (AM3): rt=1.072 min, (549.3 [M+Na]⁺), 78.2% purity.

### tert-Butyl 3-chloro-4-(trifluoromethoxy)benzyl(3-oxo-3-((3-oxopropyl)amino)propyl)carbamate 1.325

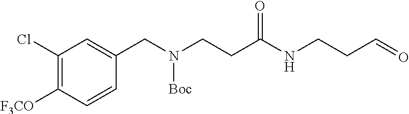 [SMILES: CC(C)(C)OC(=O)N(CCC(=O)NCCC=O)Cc1ccc(OC(F)(F)F)c(Cl)c1]

A mixture of compound 1.324 (533 mg, 1.01 mmol) in a solution of AcOH (6 mL, 104.91 mmol), THF (3 mL) and water (3 mL) was stirred at 20° C. for 1 h. The mixture was basified to pH=8 (sat. NaHCO₃ (aq.)) and then extracted with EA (20 mL×3). The combined organic phases were washed (brine, 30 mL×3), dried (Na₂SO₄), filtered and concentrated in vacuo to afford compound 1.325 (513 mg) as a yellow oil.

LCMS (AM3): rt=0.975 min, (453.1 [M+H]⁺), 82.9% purity.

### tert-Butyl 3-chloro-4-(trifluoromethoxy)benzyl(3-oxo-3-((3-((6-(pyridin-4-yl)-1-(tetrahydro-2H-pyran-2-yl)-1H-indazol-4-yl)amino)propyl)amino)propyl)carbamate 1.326

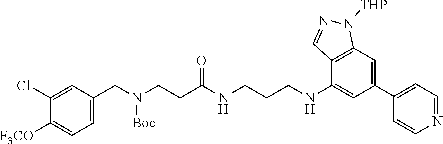

A solution of compound 1.322 (100 mg, 339.73 μmol) and compound 1.325 (230.77 mg, 509.60 μmol) in DCE (5 mL) was stirred at 20° C. for 2 h, then NaBH(AcO)₃ (216.01 mg, 1.02 mmol) was added. The mixture was stirred at 20° C. for 12 h. The mixture was concentrated in vacuo and the residue was purified (PM61) to afford compound 1.326 (240 mg, 299.53 μmol, 88.2% yield, FA salt) as a yellow solid.

LCMS (AM3): rt=0.840 min, (731.4 [M+H]⁺), 97.3% purity.

### Synthesis of Intermediate 1.328

### tert-Butyl (3-((3-((6-(4H-1,2,4-triazol-4-yl)-1H-indazol-4-yl)amino)-3-oxopropyl)amino)-3-oxopropyl)((2-chloro-[1,1′-biphenyl]-4-yl)methyl)carbamate 1.328

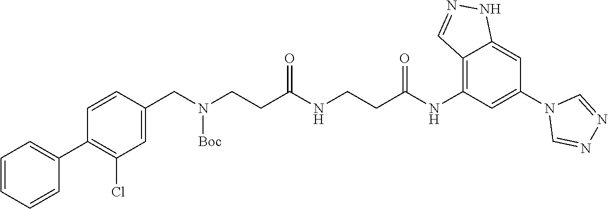 [SMILES: CC(C)(C)OC(=O)N(CCC(=O)NCCC(=O)Nc1cc(-n2cnnc2)cc2[nH]ncc12)Cc1ccc(-c2ccccc2)c(Cl)c1]

To a solution of Intermediate D (158.09 mg, 405.49 μmol) and compound 1.285 (110 mg, 405.49 μmol) in pyridine (2 mL) was added EDCI (155 mg, 810.98 μmol) at 30° C.

The mixture was stirred at 30° C. for 1 h. The mixture was concentrated in vacuo and the residue was purified (PM61) to afford compound 1.328 (60 mg, 86.76 μmol, 21.4% yield) as a yellow solid.

LCMS (AM3): rt=0.931 min, (643.3 [M+H]⁺), 87.4% purity.

### Synthesis of Intermediate 1.363

### 4-Bromo-6-nitro-1-(tetrahydro-2H-pyran-2-yl)-1H-indazole 1.356

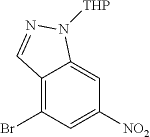

To a mixture of 4-bromo-6-nitro-1H-indazole (CAS 885518-54-7, 1 g, 4.13 mmol) and 3,4-dihydro-2H-pyran (521.32 mg, 6.20 mmol) in THF (10 mL) was added TsOH·H₂O (65 mg, 341.71 μmol) at rt. The mixture was heated to 50° C. and stirred for 12 h. Sat. NaHCO₃ (aq.) solution (20 mL) was added to the mixture and the mixture was extracted with EA (30 mL×3). The combined organic phase was washed (brine, 80 mL), dried (Na₂SO₄), filtered and concentrated in vacuo. The residue was purified (PM7) to afford compound 1.356 (1.3 g, 3.99 mmol, 96.5% yield) as a white solid.

¹H NMR (400 MHz, CHCl₃-d) δ: 8.55 (dd, J=1.6, 0.8 Hz, 1H), 8.22 (d, J=1.6 Hz, 1H), 8.14 (d, J=0.8 Hz, 1H), 5.82 (dd, J=8.8, 2.8 Hz, 1H), 4.05-4.00 (m, 1H), 3.83-3.77 (m, 1H), 2.56-2.46 (m, 1H), 2.21-2.12 (m, 2H), 1.92-1.68 (m, 3H) ppm.

### 6-Nitro-1-(tetrahydro-2H-pyran-2-yl)-4-(4,4,5,5-tetramethyl-1,3,2-dioxaborolan-2-yl)-1H-indazole 1.357

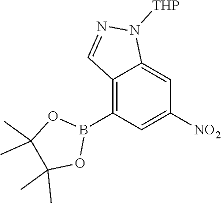

To a mixture of compound 1.356 (1.3 g, 3.99 mmol) and 4,4,4′,4′,5,5,5′,5′-octamethyl-2,2′-bi(1,3,2-dioxaborolane) (1.11 g, 4.38 mmol) in toluene (20 mL) was added KOAc (782.38 mg, 7.97 mmol) and Pd(dppf)Cl₂ (291.65 mg, 398.60 μmol) at RT. The mixture was heated to 80° C. and stirred for 16 h under N₂. The mixture was concentrated in vacuo and the residue was purified (PM7) to afford compound 1.357 (1.4 g, 3.75 mmol, 94.1% yield) as a colourless oil.

¹H NMR (400 MHz, CHCl₃-d) δ: 8.63 (s, 1H), 8.52-8.51 (d, 2H), 5.83 (dd, J=2.4, 8.8 Hz, 1H), 4.06-4.01 (m, 1H), 3.83-3.77 (m, 1H), 2.58-2.49 (m, 1H), 2.22-2.08 (m, 2H), 1.88-1.67 (m, 3H), 1.42 (s, 12H) ppm.

### 6-Nitro-1-(tetrahydro-2H-pyran-2-yl)-1H-indazol-4-ol 1.358

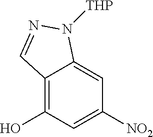

To a mixture of compound 1.357 (500 mg, 1.34 mmol) in EtOH (10 mL) was added hydroxylamine hydrochloride (279.3 mg, 4.02 mmol) and NaOH (202.3 mg, 2.68 mmol) at 35° C. The mixture was stirred at 35° C. for 19 h. The mixture was adjusted to pH=6 by adding HCl (aq.) solution (1 N) and diluted with water (40 mL). The aqueous phase was extracted with EA (40 mL×3). The combined organic phase was washed (brine, 100 mL), dried (Na₂SO₄), filtered and concentrated in vacuo. The residue was purified (PM5) to afford compound 1.358 (300 mg, 1.14 mmol, 42.5% yield) as a yellow solid.

¹H NMR (400 MHz, DMSO-d₆) δ: 11.28 (s, 1H), 8.28 (s, 1H), 8.14 (s, 1H), 7.26 (d, J=2.0 Hz, 1H), 6.01 (dd, J=10.0, 2.4 Hz, 1H), 3.88-3.77 (m, 2H), 2.43-2.32 (m, 1H), 2.05-1.96 (m, 2H), 1.81-1.70 (m, 1H), 1.63-1.52 (m, 2H) ppm.

### 6-Amino-1-(tetrahydro-2H-pyran-2-yl)-1H-indazol-4-ol 1.359

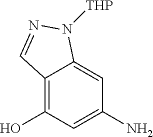

To a solution of compound 1.358 (900 mg, 3.42 mmol) in MeOH (50 mL) was added 10% Pd/C (150 mg) under N₂ protection. The suspension was degassed and purged with H₂ (×3) at 20° C. The mixture was stirred at 20° C. for 3 h under an H₂ atmosphere (15 psi). The mixture was filtered and the filtrate was concentrated in vacuo to afford compound 1.359 (900 mg) as a brown solid, which was used directly without purification.

LCMS (AM3): rt=0.619 min, (234.2 [M+H]⁺), 93.8% purity.

### 1-(Tetrahydro-2H-pyran-2-yl)-6-(4H-1,2,4-triazol-4-yl)-1H-indazol-4-ol 1.360

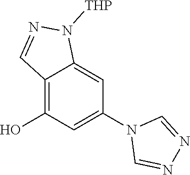

To a mixture of compound 1.359 (900 mg, 3.86 mmol), N,N′-diformylhydrazine (1.70 g, 19.29 mmol) and TEA (2.73 g, 27.01 mmol) in pyridine (20 mL) was added TMSCl (6.29 g, 57.87 mmol) at rt. The mixture was then heated to 120° C. and stirred for 12 h under N₂ protection. The mixture was concentrated in vacuo and the residue was purified (PM67) to afford compound 1.360 (820 mg, 2.87 mmol, 74.5% yield) as a yellow solid.

LCMS (AM3): rt=0.742 min, (286.5 [M+H]⁺), 58.1% purity.

### tert-Butyl (3-((1-(tetrahydro-2H-pyran-2-yl)-6-(4H-1,2,4-triazol-4-yl)-1H-indazol-4-yl)oxy)propyl)carbamate 1.361

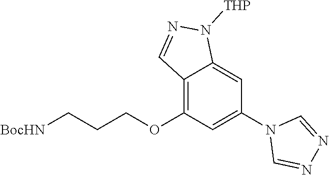

A mixture of compound 1.360 (400 mg, 1.40 mmol), tert-butyl (3-bromopropyl)carbamate (500.78 mg, 2.10 mmol) and K₂CO₃ (387.55 mg, 2.80 mmol) in DMF (10 mL) was stirred at 80° C. for 16 h. The mixture was diluted with water (30 mL) and the aqueous phase was extracted with EA (30 mL×2). The combined organic phases were washed (brine, 50 mL), dried (Na₂SO₄), filtered and concentrated in vacuo. The residue was purified (PM95) to afford compound 1.361 (500 mg, 1.13 mmol, 80.6% yield) as a white solid.

LCMS (AM3): rt=0.888 min, (443.1 [M+H]⁺), 100% purity.

### 3-((6-(4H-1,2,4-triazol-4-yl)-1H-indazol-4-yl)oxy)propan-1-amine 1.362

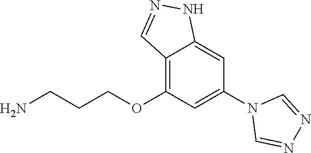 [SMILES: NCCCOc1cc(-n2cnnc2)cc2[nH]ncc12]

To a mixture of compound 1.361 (500 mg, 1.13 mmol) in 1,4-dioxane (10 mL) was added a solution of HCl in 1,4-dioxane (4 M, 10 mL) at 20° C. The mixture was stirred at 20° C. for 3 h. The mixture was concentrated in vacuo to afford compound 1.362 (340 mg, HCl salt) as a white solid, which was used directly without purification.

LCMS (AM6): rt=1.209 min, (259.2 [M+H]⁺), 91.8% purity.

### tert-Butyl (3-((3-((6-(4H-1,2,4-triazol-4-yl)-1H-indazol-4-yl)oxy)propyl)amino)-3-oxopropyl)(3-chloro-4-(trifluoromethoxy)benzyl)carbamate 1.363

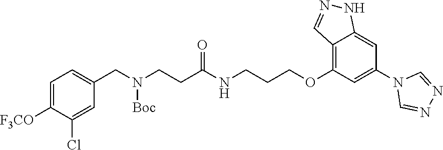 [SMILES: CC(C)(C)OC(=O)N(CCC(=O)NCCCOc1cc(-n2cnnc2)cc2[nH]ncc12)Cc1ccc(OC(F)(F)F)c(Cl)c1]

A mixture of compound 1.243 (150 mg, 377.10 μmol), HATU (172.06 mg, 452.52 μmol) and DIPEA (194.95 mg, 1.51 mmol) in DMF (3 mL) was stirred at 30° C. for 0.5 h, then compound 1.362 (100 mg, 339.28 μmol) was added. The mixture was stirred at 30° C. for 2 h. The mixture was diluted with water (30 mL) and extracted with EA (30 mL×3). The combined organic phase was washed (brine, 90 mL), dried (Na₂SO₄), filtered and concentrated in vacuo to afford compound 1.363 (300 mg) as a brown oil which was used without further purification.

LCMS (AM3): rt=0.959 min, (638.1 [M+H]⁺), 45.6% purity.

### Synthesis of Intermediate 1.371

### tert-Butyl 2-(((4-(2-((6-(4H-1,2,4-triazol-4-yl)-1H-indazol-4-yl)amino)ethoxy)butyl)amino)methyl)-6-chloro-1H-benzo[d]imidazole-1-carboxylate 1.371

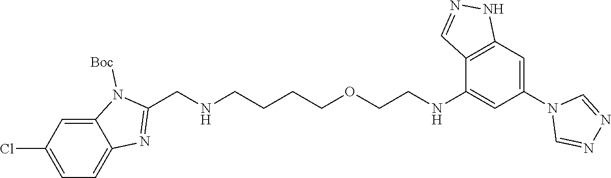 [SMILES: CC(C)(C)OC(=O)n1c(CNCCCCOCCNc2cc(-n3cnnc3)cc3[nH]ncc23)nc2ccc(Cl)cc21]

To a mixture of compound 1.306 (300 mg, 951.26 μmol) and K₂CO₃ (394.41 mg, 2.85 mmol) in DMF (30 mL) was added 2-chloromethyl-1-tert-butoxycarbonyl-6-chlorobenzimidazole (CAS 305357-46-4, 143.24 mg, 475.63 μmol), then the reaction mixture was heated to 50° C. and stirred for 2 h. The reaction mixture was diluted with H₂O (50 mL) and extracted with EA (80 mL). The organic layer was washed (brine, 50 mL×2), dried (Na₂SO₄), filtered and concentrated in vacuo. The crude product was purified (PM99) to afford compound 1.371 (60 mg, 103.43 μmol, 10.9% yield) as a brown solid.

LCMS (AM7): rt=0.899 min, (580.3 [M+H]⁺), 24.0% purity.

### Synthesis of Intermediate 1.374

### tert-Butyl (4-(2-iodoethoxy)butyl)carbamate 1.372

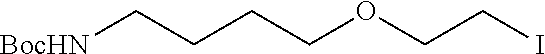 [SMILES: CC(C)(C)OC(=O)NCCCCOCCI]

To a mixture of tert-butyl (4-(2-hydroxyethoxy)butyl)carbamate (WO2022185041, 1.3 g, 5.57 mmol), imidazole (569.03 mg, 8.36 mmol) and PPh₃ (2.19 g, 8.36 mmol) in DCM (10 mL) was added iodine (2.12 g, 8.36 mmol) at 0° C. under N₂. The mixture was warmed to RT and stirred for 12 h. The excess iodine was quenched with sat. Na₂SO₃ (aq.) (30 mL) and the mixture was extracted with EA (50 mL×3). The combined organic phase was washed (brine, 50 mL), dried (Na₂SO₄), filtered and concentrated in vacuo. The residue was purified (PM7) to afford compound 1.372 (1.4 g, 4.08 mmol, 73.2% yield) as a yellow oil.

¹H NMR (400 MHz, MeOH-d₄) δ: 3.70 (t, J=6.4 Hz, 2H), 3.52 (t, J=6.0 Hz, 2H), 3.31 (t, J=6.4 Hz, 2H), 3.08 (t, J=6.8 Hz, 2H), 1.65-1.54 (m, 4H), 1.45 (s, 9H) ppm.

### tert-Butyl (4-(2-((1-(tetrahydro-2H-pyran-2-yl)-6-(4H-1,2,4-triazol-4-yl)-1H-indazol-4-yl)oxy)ethoxy)butyl)carbamate 1.373

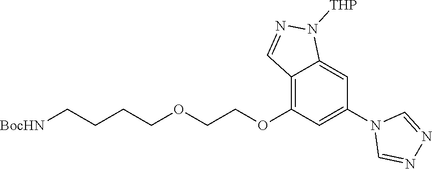

To a mixture of compound 1.360 (200 mg, 701.01 μmol) and K₂CO₃ (193.77 mg, 1.40 mmol) in DMF (2 mL) was added compound 1.372 (360.88 mg, 1.05 mmol), the mixture was heated to 80° C. and stirred for 12 h. The reaction mixture was concentrated in vacuo to give a residue which was purified (PM61) to afford compound 1.373 (300 mg, 545.36 μmol, 77.8% yield) as a brown solid.

LCMS (AM3): rt=0.730 min, (501.1 [M+H]⁺),72.6% purity.

### 4-(2-((6-(4H-1,2,4-triazol-4-yl)-1H-indazol-4-yl)oxy)ethoxy)butan-1-amine 1.374

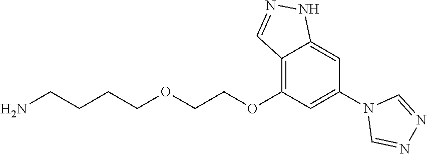 [SMILES: NCCCCOCCOc1cc(-n2cnnc2)cc2[nH]ncc12]

A mixture of compound 1.373 (0.3 g, 599.29 μmol) in a solution of HCl in 1,4-dioxane (4 M, 10 mL) was stirred at 20° C. for 2 h. The reaction mixture was concentrated in vacuo to afford compound 1.374 (0.2 g, HCl salt) as a brown solid, which was used directly without purification.

LCMS (AM3): rt=0.445 min, (316.9 [M+H]⁺), 85.8% purity.

### Synthesis of Intermediate 1.383

### 6-Bromo-1-(tetrahydro-2H-pyran-2-yl)-1H-indazol-4-amine 1.381

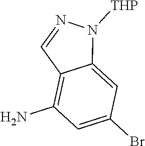

To a solution of NiCl₂·6H₂O (400 mg, 1.69 mmol) in MeOH (10 mL) and THF (20 mL) was added NaBH₄ (63 mg, 1.69 mmol) at 0° C. After stirring for 5 min, 6-bromo-4-nitro-1-(tetrahydro-2H-pyran-2-yl)-1H-indazole (Ref: WO2019027960, 1.1 g, 3.37 mmol) was added, followed by addition of NaBH₄ (382 mg, 10.12 mmol). The mixture was stirred at 0° C. for 25 min. The mixture was quenched with water (100 mL) and filtered, and the filtrate was extracted with EA (50 mL×2). The combined organic phases were washed (brine, 50 mL), dried (Na₂SO₄) and concentrated in vacuo to afford compound 1.381 (990 mg, 3.03 mmol, 89.8% yield) as a yellow oil.

LCMS (AM3): rt=0.877 min, (296.0 [M+H]⁺), 90.6% purity.

### tert-Butyl (3-((3-((6-bromo-1-(tetrahydro-2H-pyran-2-yl)-1H-indazol-4-yl)amino)propyl)amino)-3-oxopropyl)((2-chloro-[1,1′-biphenyl]-4-yl)methyl)carbamate 1.382

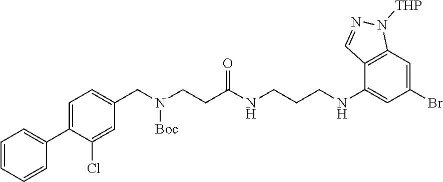

To a solution of compound 1.381 (200 mg, 0.67 mmol) in MeOH (10 mL) was added Intermediate H (360 mg, 0.81 mmol) at 25° C. and the reaction mixture stirred for 11 h. NaBH₃CN (346 mg, 5.52 mmol) was added and the mixture stirred at 25° C. for 1 h. The mixture was concentrated in vacuo and the crude product purified (PM69) to afford compound 1.382 (150 mg, 0.20 mmol, 36.5% yield) as a light-yellow oil.

LCMS (AM3): rt=1.114 min, (726.1 [M+H]⁺), 96.1% purity.

### tert-Butyl ((2-chloro-[1,1′-biphenyl]-4-yl)methyl)(3-((3-((6-(2-methylpyridin-4-yl)-1-(tetrahydro-2H-pyran-2-yl)-1H-indazol-4-yl)amino)propyl)amino)-3-oxopropyl)carbamate 1.383

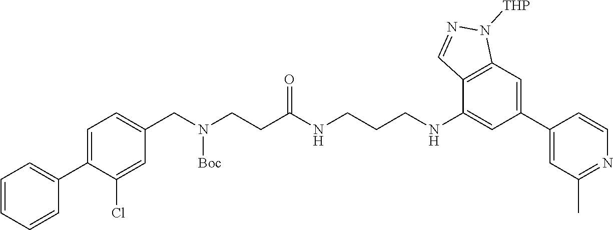

A mixture of compound 1.382 (150 mg, 0.206 mmol), K₂CO₃ (57 mg, 0.413 mmol), (2-methylpyridin-4-yl)boronic acid (34 mg, 0.248 mmol) and Brettphos G3 Pd (19 mg, 0.020 mmol) in 1,4-dioxane (2 mL) and H₂O (0.2 mL) was stirred at 90° C. for 0.5 h under microwave irradiation. The mixture was concentrated in vacuo and the crude product was purified (PM67) to afford compound 1.383 (90 mg, 0.121 mmol, 58.3% yield) as a yellow solid.

LCMS (AM3): rt=0.947 min, (737.2 [M+H]⁺), 98.9% purity.

### Synthesis of Intermediate 1.384

### tert-Butyl ((2-chloro-[1,1′-biphenyl]-4-yl)methyl)(3-((3-((6-(2-methoxypyridin-4-yl)-1-(tetrahydro-2H-pyran-2-yl)-1H-indazol-4-yl)amino)propyl)amino)-3-oxopropyl)carbamate 1.384

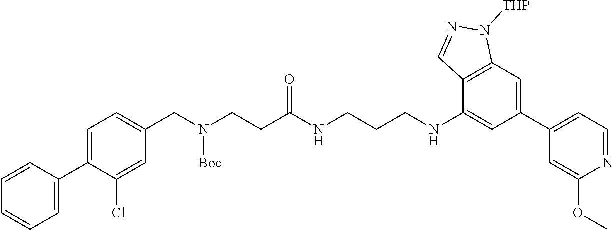

A solution of compound 1.382 (150 mg, 0.206 mmol), K₂CO₃ (57 mg, 0.413 mmol), (2-methoxypyridin-4-yl)boronic acid (38 mg, 0.248 mmol) and Brettphos G3 Pd (19 mg, 0.020 mmol) in 1,4-dioxane (2 mL) and H₂O (0.2 mL) was stirred at 90° C. for 0.5 h under microwave irradiation and under N₂. The mixture was concentrated in vacuo and purified (PM67) to afford compound 1.384 (100 mg, 0.118 mmol, 57.4% yield) as a yellow solid.

LCMS (AM3): rt=1.108 min, (753.3 [M+H]⁺), 89.5% purity.

### Synthesis of Intermediate 1.420

### Methyl 5-cyclobutoxy-1H-indole-2-carboxylate 1.418

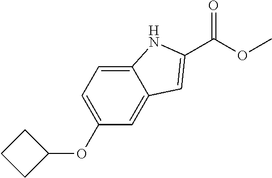 [SMILES: COC(=O)c1cc2cc(OC3CCC3)ccc2[nH]1]

A mixture of methyl 5-hydroxy-1H-indole-2-carboxylate (850 mg, 4.45 mmol, Bloorganic and Medicinal Chemistry Letters, 2000, 10 (5), 483-486), bromocyclobutane (1.70 mL, 18.01 mmol) and K₂CO₃ (1.53 g, 11.07 mmol) in DMF (15 mL) was stirred at 80° C. for 20 h. The reaction mixture was poured into H₂O (45 mL), extracted with EA (20 mL×2), the combined organic phase washed (brine, 40 mL), dried (Na₂SO₄), filtered and concentrated in vacuo. The residue was purified (PM8) to afford compound 1.418 (700 mg, 60.3% yield) as a yellow solid.

LCMS (AM3): rt=0.942 min, (246.3 [M+H]⁺), 93.9% purity.

### (5-Cyclobutoxy-1H-indol-2-yl)methanol 1.419

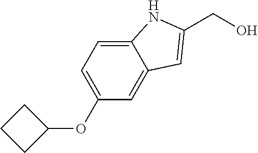 [SMILES: OCc1cc2cc(OC3CCC3)ccc2[nH]1]

To a mixture of LAH (260 mg, 6.85 mmol) in THF (20 mL) was added compound 1.418 (650 mg, 2.65 mmol) in THF (5 mL) at 0° C. The reaction mixture was warmed to RT, stirred for 2 h, then the reaction quenched with H₂O (0.5 mL) followed by aq. 10% NaOH solution (0.5 mL) and H₂O (1 mL). The reaction mixture was stirred for 1 h, then Na₂SO₄ added, the mixture filtered and the filtrate concentrated in vacuo to afford compound 1.419 (470 mg, 81.6% yield) as a white solid, which was used directly without purification.

LCMS (AM3): rt=0.840 min, (218.3 [M+H]⁺), 90.3% purity.

### 5-Cyclobutoxy-1H-indole-2-carbaldehyde 1.420

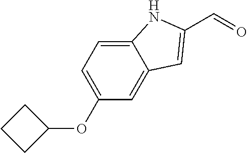 [SMILES: O=Cc1cc2cc(OC3CCC3)ccc2[nH]1]

A mixture of compound 1.419 (470 mg, 2.16 mmol) and MnO₂ (1.88 g, 21.63 mmol) in DCM (20 mL) was stirred at RT for 12 h. The reaction mixture was filtered and the filtrate was concentrated in vacuo. The residue was purified (PM7) to afford compound 1.420 (370 mg, 58.8% yield) as a brown solid.

LCMS (AM4): rt=0.846 min, (216.1 [M+H]⁺), 74.2% purity.

### Synthesis of Intermediate 1.461

### tert-Butyl (5-(cyanomethoxy)pentyl)carbamate 1.460

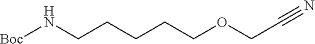 [SMILES: CC(C)(C)OC(=O)NCCCCCOCC#N]

To a solution of tert-butyl (5-hydroxypentyl)carbamate (4 g, 19.68 mmol) in DCM (50 mL) was added 2-bromoacetonitrile (4.72 g, 39.36 mmol), TBAI (1.45 g, 3.94 mmol) and Ag₂O (4.56 g, 19.68 mmol) at 25° C. The reaction mixture was stirred at 25° C. for 12 h. The mixture was filtered and concentrated in vacuo, and the residue was purified (PM8) to afford compound 1.460 (280 mg, 1.16 mmol, 5.9% yield) as a colourless oil.

¹H NMR (400 MHz, CHCl₃-d) δ: 4.56 (br s, 1H), 4.23 (s, 2H), 3.57 (t, J=6.4 Hz, 2H), 3.11 (q, J=6.0 Hz, 2H), 1.67-1.60 (quin, 2H), 1.54-1.42 (m, 2H), 1.44 (s, 9H), 1.42-1.35 (m, 2H) ppm.

### tert-Butyl (5-(2-aminoethoxy)pentyl)carbamate 1.461

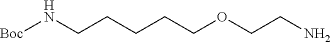 [SMILES: CC(C)(C)OC(=O)NCCCCCOCCN]

To a solution of compound 1.460 (400 mg, 1.65 mmol) in MeOH (10 mL) was added Raney Ni (141.43 mg, 1.65 mmol) and ammonium hydroxide (826.46 mg, 6.60 mmol, 28%) under N₂. The suspension was degassed and purged with H₂ (×3). The mixture was hydrogenated under H₂ at 25° C. for 12 h. The catalyst was removed by filtration and the filtrate was concentrated in vacuo to give compound 1.461 (423 mg) as a light yellow oil, which was used without purification.

### Synthesis of Intermediate 1.848

### tert-Butyl (S)-2-((4-((tert-butoxycarbonyl)amino)pentyl)oxy)acetate 1.847

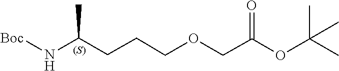

To a solution of tert-butyl (S)-(5-hydroxypentan-2-yl)carbamate (5 g, 24.60 mmol, Ref: ACS Medicinal Chemistry Letters, 2021, vol. 12, #1, p. 121-128) and tert-butyl 2-bromoacetate (9.90 g, 50.76 mmol) in NaOH aq. (10 g dissolved in 25 mL water) was added TBAI (500 mg, 1.35 mmol) at 0° C. and stirred at 0° C. for 2 h. The reaction mixture was diluted with H₂O (80 mL) and extracted (MTBE, 80 mL×2). The combined organic phases were washed (brine, 100 mL), dried (Na₂SO₄), filtered and concentrated in vacuo to give a residue which was purified (PM56) to afford compound 1.847 (7 g, 22.05 mmol, 89.6% yield) as a colourless oil.

¹H NMR (400 MHz, CHCl₃-d) δ: 4.37 (s, 1H) 3.94 (s, 2H) 3.64 (s, 1H) 3.51 (t, J=6.0 Hz, 2H) 1.70-1.61 (m, 2H) 1.54-1.50 (m, 2H) 1.48 (s, 9H) 1.43 (s, 9H) 1.12 (d, J=6.4 Hz, 3H) ppm.

### tert-Butyl (S)-(5-(2-hydroxyethoxy)pentan-2-yl)carbamate 1.848

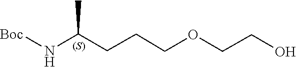

To a mixture of LAH (900.00 mg, 23.71 mmol) in THF (50 mL) at 0° C. was added slowly a solution of compound 1.847 (6 g, 18.90 mmol) in THF (50 mL). The reaction mixture was stirred at 0° C. for 2 h. The reaction mixture was quenched slowly at 0° C. with H₂O (1 mL), NaOH aq.(15% wt., 1 mL) then H₂O (3 mL) and stirred for 0.5 h. Na₂SO₄ (10 g) was added, and the resulting suspension stirred for 0.5 h then filtered. The filter cake was washed with EA (100 mL). The filtrate was concentrated in vacuo to give a residue which was purified (PM3) to afford compound 1.848 (3.5 g, 14.15 mmol, 74.8% yield) as a colourless oil.

¹H NMR (400 MHz, CHCl₃-d) δ: 3.75-3.72 (m, 2H), 3.70-3.65 (m, 1H), 3.55-3.52 (m, 2H), 3.51-3.48 (m, 2H), 1.69-1.60 (m, 2H), 1.53-1.48 (m, 2H), 1.45 (s, 9H), 1.13 (d, J=6.4 Hz, 3H) ppm.

### Synthesis of Intermediate 1.856

### tert-Butyl 2-((3-((tert-butoxycarbonyl)amino)cyclobutyl)methoxy)acetate 1.855

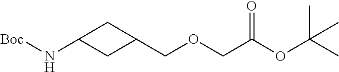 [SMILES: CC(C)(C)OC(=O)COCC1CC(NC(=O)OC(C)(C)C)C1]

To a solution of tert-butyl (3-(hydroxymethyl)cyclobutyl)carbamate (CAS 167081-37-0, 5 g, 24.84 mmol), tert-butyl 2-bromoacetate (9.69 g, 49.69 mmol) in NaOH aq. (10 g dissolved in 25 mL water) was added TBAI (458.81 mg, 1.24 mmol) at 0° C. The reaction mixture was stirred at 0° C. for 2 h, then diluted with water (100 mL) and extracted (MTBE, 100 mL×2). The combined organic phases were washed (brine, 150 mL), dried (Na₂SO₄), filtered and concentrated in vacuo to give a residue which was purified (PM56) to afford compound 1.855 (9.8 g, crude) as colourless oil.

¹H NMR (400 MHz, CHCl₃-d) δ: 4.88-4.63 (m, 1H), 4.25-4.15 (m, 0.5H), 4.08-3.96 (m, 0.5H), 3.94 (d, J=6.0 Hz, 2H), 3.54 (d, J=7.2 Hz, 1H), 3.45 (d, J=5.6 Hz, 1H), 2.48-2.43 (m, 1H), 2.27-2.19 (m, 1.5H), 2.02-1.96 (m, 1H), 1.76-1.54 (m, 1.5H), 1.48 (s, 9H) 1.42 (s, 9H) ppm.

### tert-Butyl (3-((2-hydroxyethoxy)methyl)cyclobutyl)carbamate 1.856

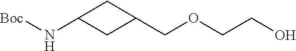 [SMILES: CC(C)(C)OC(=O)NC1CC(COCCO)C1]

To a solution of compound 1.855 (3 g, 9.51 mmol) in cooled THF (40 mL) at 0° C. was added LAH (400 mg, 10.54 mmol) slowly. The reaction mixture was stirred at 0° C. for 2 h. The reaction mixture was quenched slowly with water (0.4 mL). 15% wt. aq. NaOH solution (0.4 mL) and water (1.2 mL) were added at 0° C. and the mixture stirred for 0.5 h. Na₂SO₄ (5 g) was added. The resulting suspension was stirred for 0.5 h, filtered and concentrated in vacuo to give a residue which was purified (PM3) to afford compound 1.856 (1 g, 4.08 mmol, 42.8% yield) as colourless oil.

¹H NMR (400 MHz, CHCl₃-d) δ: 4.74 (s, 1H), 4.28-4.15 (m, 0.5H), 4.05-3.95 (m, 0.5H), 3.75-3.72 (m, 2H), 3.57-3.54 (m, 2H), 3.53-3.51 (m, 1H), 3.42 (d, J=5.6 Hz, 1H), 2.49-2.41 (m, 1.5H), 2.25-2.16 (m, 1.5H), 2.02-1.95 (m, 1H), 1.66-1.58 (m, 1H), 1.44 (s, 9H) ppm.

### Synthesis of Intermediate 1.888

### tert-butyl ((2-chloro-[1,1′-biphenyl]-4-yl)methyl)(3-oxo-3-((3-((6-(pyridin-3-yl)-1-(tetrahydro-2H-pyran-2-yl)-1H-indazol-4-yl)amino)propyl)amino)propyl)carbamate 1.888

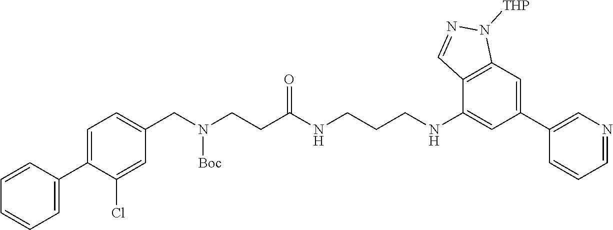

To a solution of compound 1.382 (150 mg, 206.86 μmol) in H₂O (0.2 mL) and DME (2 mL) was added K₂CO₃ (57.18 mg, 413.73 μmol), 3-pyridylboronic acid (30.51 mg, 248.24 mol) and Pd(PPh₃)₄(23.9 mg, 20.69 μmol). The mixture was stirred at 90° C. for 0.5 h under microwave irradiation. The mixture was concentrated in vacuo to give a crude residue, which was purified (PM100) to afford compound 1.888 (100 mg, 121.39 μmol, 58.68% yield) as a yellow solid.

LCMS (AM3): rt=0.904 min, (723.2 [M+H]⁺), 87.81% purity.

### Synthesis of Intermediate 1.889

### tert-butyl ((2-chloro-[1,1′-biphenyl]-4-yl)methyl)(3-((3-((6-(3-methyl-1H-pyrazol-4-yl)-1-(tetrahydro-2H-pyran-2-yl)-1H-indazol-4-yl)amino)propyl)amino)-3-oxopropyl)carbamate 1.889

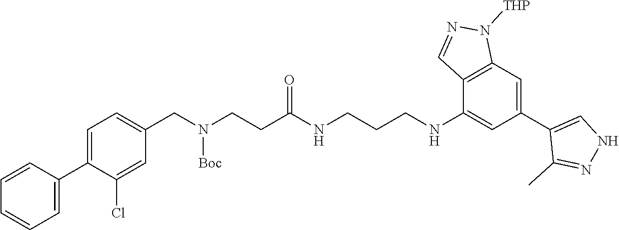

To a solution of compound 1.382 (200 mg, 275.82 μmol) in dioxane (2 mL) and H₂O (0.2 mL) was added K₂CO₃ (76.24 mg, 551.64 μmol), 3-methyl-1H-pyrazol-4-yl)boronic acid (41.68 mg, 330.98 μmol) and Pd(dppf)Cl₂ (20.18 mg, 27.58 μmol). The mixture was stirred at 90° C. for 0.5 h under microwave irradiation. The solid was filtered and the filtrate was concentrated in vacuo to give a residue, which was purified (PM101) to afford compound 1.889 (65 mg, 79.38 μmol, 28.78% yield) as a light-yellow oil.

LCMS (AM3): rt=0.984 min, (726.2 [M+H]⁺), 88.7% purity.

### Synthesis of Intermediate 1.890

### tert-butyl ((2-chloro-[1,1′-biphenyl]-4-yl)methyl)(3-((3-((6-(isoxazol-4-yl)-1-(tetrahydro-2H-pyran-2-yl)-1H-indazol-4-yl)amino)propyl)amino)-3-oxopropyl)carbamate 1.890

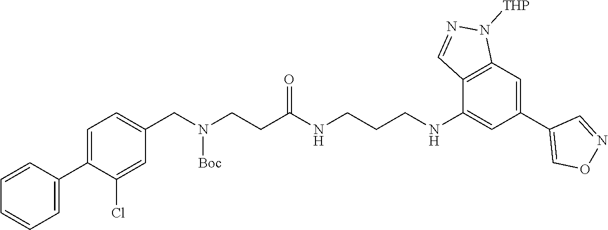

To a solution of compound 1.382 (100 mg, 137.91 μmol) in dioxane (2 mL) and H₂O (0.2 mL) was added Pd(dppf)Cl₂ (10.09 mg, 13.79 μmol), isoxazol-4-ylboronic acid (18.68 mg, 165.49 μmol) and K₃PO₄ (58.55 mg, 275.82 μmol). The mixture was stirred at 80° C. for 0.5 h under microwave irradiation. The solid was filtered and the filtrate was concentrated in vacuo to give a residue, which was purified (PM110) to afford compound 1.890 (90 mg, 52.74 mol, 38.25% yield) as a light-yellow oil.

LCMS (AM3): rt=1.095 min, (713.2 [M+H]⁺), 41.8% purity.

### Synthesis of Intermediate 1.895

### 2-(4-((tert-butoxycarbonyl)amino)butoxy)acetic acid 1.891

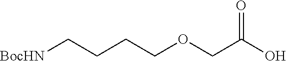 [SMILES: CC(C)(C)OC(=O)NCCCCOCC(=O)O]

To a solution of tert-butyl (4-hydroxybutyl)carbamate (CAS 75178-87-9, 20 g, 105.68 mmol) and 2-bromoacetic acid (13.33 g, 95.96 mmol) in THF (300 mL) at 0° C. was added NaH (8.06 g, 201.51 mmol, 60% purity). The mixture was warmed to 25° C. and stirred for 16 h. The reaction mixture was poured onto NaOH (aq.) (1 N, 200 mL). The mixture was extracted with DCM (150 mL×3). The aqueous phase was adjusted to pH=3 (conc. HCl) and the mixture extracted with DCM (300 mL×3). The combined organic phase was washed (brine, 50 mL), dried (Na₂SO₄), filtered and concentrated in vacuo to afford compound 1.891 (18 g, 72.79 mmol, 75.85% yield) as a yellow oil.

¹H NMR: (400 MHz, CHCl₃-d) δ: 10.88 (s, 1H), 4.08 (s, 2H), 3.55 (t, J=6.0 Hz, 2H), 3.13 (s, 2H), 1.89-1.52 (m, 4H), 1.42 (s, 9H) ppm

### tert-butyl (4-(2-((6-bromo-1-(tetrahydro-2H-pyran-2-yl)-1H-indazol-4-yl)amino)-2-oxoethoxy)butyl)carbamate 1.892

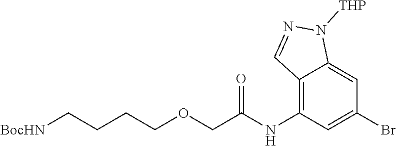

To a solution of compound 1.381 (3.4 g, 11.48 mmol) in pyridine (100 mL) was added EDCI (22.01 g, 114.80 mmol) and compound 1.891 (3.12 g, 12.63 mmol). The mixture was stirred at 100° C. for 12 h. The mixture was concentrated in vacuo to give a residue, which was purified (PM66) to afford compound 1.892 (5.5 g, 10.47 mmol, 85.0% yield) as a light-yellow oil.

LCMS (AM3): rt=0.927 min, (525.2 [M+H]⁺), 93.3% purity.

### tert-butyl (4-(2-((6-bromo-1-(tetrahydro-2H-pyran-2-yl)-1H-indazol-4-yl)amino)ethoxy)butyl)carbamate 1.893

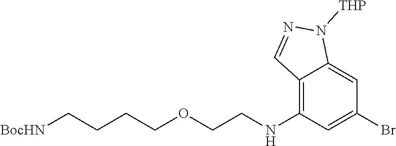

To a solution of compound 1.892 (5.5 g, 10.47 mmol) in THF (50 mL) at 0° C. was added borane; methylsulfanylmethane (10 M, 10.47 mL). The mixture was warmed to 25° C. and stirred for 2 h. MeOH (40 mL) was added slowly, the mixture was stirred at 25° C. for 1 h, the mixture was concentrated in vacuo to give a residue, which was purified (PM200) to afford compound 1.893 (4 g, 6.34 mmol, 60.67% yield) as a light-red oil.

LCMS (AM3): rt=1.017 min, (511.2 [M+H]⁺), 98.3% purity.

### (4-(2-((6-(isoxazol-4-yl)-1-(tetrahydro-2H-pyran-2-yl)-1H-indazol-4-yl)amino)ethoxy)butyl)carbamate 1.894

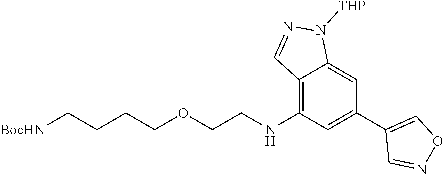

To a solution of compound 1.893 (2 g, 3.17 mmol) in dioxane (20 mL) and H₂O (2 mL) was added K₃PO₄ (1.34 g, 6.33 mmol), isoxazol-4-ylboronic acid (741.2 mg, 3.80 mmol) and [2-(2-aminophenyl)phenyl]-methylsulfonyloxypalladium; dicyclohexyl-[3,6-dimethoxy-2-(2,4,6-triisopropylphenyl)phenyl]phosphane (287.13 mg, 0.31 mmol). The mixture was stirred at 60° C. for 3 h. The mixture was concentrated in vacuo to give a residue, which was purified (PM239) to afford compound 1.894 (1.4 g, 2.8 mmol, 88.4% yield) as a light-yellow oil.

LCMS (AM3): rt=0.978 min, (500.2 [M+H]⁺), 100% purity.

### N-(2-(4-aminobutoxy)ethyl)-6-(isoxazol-4-yl)-1H-indazol-4-amine 1.895

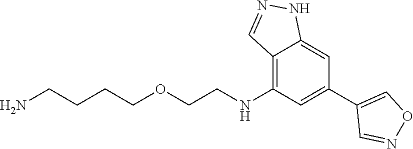 [SMILES: NCCCCOCCNc1cc(-c2cnoc2)cc2[nH]ncc12]

A solution o compound. mg, 1.40 mmol) in HCl/dioxane (4 M, 10 mL) was stirred at 25° C. for 2 h. The mixture was concentrated in vacuo to give a residue, which was purified (PM240) to afford compound 1.895 (130 mg, 0.363 mmol, 25.9% yield, HCl salt) as a light-yellow solid.

LCMS (AM3): rt=0.678 min, (316.2 [M+H]⁺), 98.3% purity

### Synthesis of Intermediate 1.900

### 6-bromo-N,N-dimethyl-4-nitro-1H-benzo[d][1,2,3]triazole-1-sulfonamide 1.897

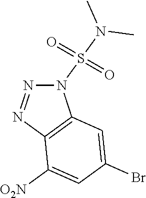 [SMILES: CN(C)S(=O)(=O)n1nnc2c([N+](=O)[O-])cc(Br)cc21]

To a solution of 6-bromo-4-nitro-1H-benzo[d][1,2,3]triazole (WO2016168682, 850 mg, 3.50 mmol) in DCM (20 mL) at 25° C. was added TEA (1.06 g, 10.49 mmol) and dimethylsulfamoyl chloride (602.69 mg, 4.20 mmol) and the mixture stirred for 12 h. The mixture was then concentrated in vacuo to give a residue, which was purified (PM46) to afford compound 1.897 (850 mg, 2.43 mmol, 66.9% yield) as a light yellow solid.

LCMS (AM3): rt=0.931 min, (350.0 [M+H]⁺), 96.5% purity.

### N,N-dimethyl-4-nitro-6-(pyridin-4-yl)-1H-benzo[d][1,2,3]triazole-1-sulfonamide 1.898

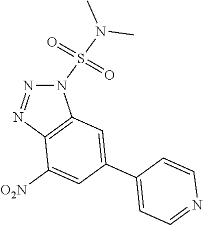 [SMILES: CN(C)S(=O)(=O)n1nnc2c([N+](=O)[O-])cc(-c3ccncc3)cc21]

To a solution of compound 1.897 (850 mg, 2.43 mmol) in dioxane (5 mL) and H₂O (0.5 mL) was added Pd(dppf)Cl₂ (177.63 mg, 0.24 mmol), pyridin-4-ylboronic acid (358.06 mg, 2.91 mmol) and K₂CO₃ (671.00 mg, 4.86 mmol) under N₂. The mixture was stirred at 80° C. for 0.5 h under microwave irradiation. The mixture was neutralized to pH=7 with formic acid, then concentrated in vacuo to give a residue, which was purified (PM41) to afford compound 1.898 (170 mg, 0.40 mmol, 16.5% yield) as a light-yellow solid.

LCMS (AM3): rt=0.465 min, (348.8 [M+H]⁺), 82.1% purity.

### 4-amino-N,N-dimethyl-6-(pyridin-4-yl)-1H-benzo[d][1,2,3]triazole-1-sulfonamide 1.899

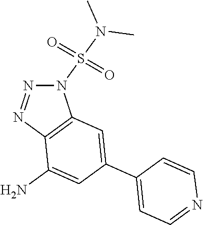 [SMILES: CN(C)S(=O)(=O)n1nnc2c(N)cc(-c3ccncc3)cc21]

To a solution of compound 1.898 (170 mg, 0.40 mmol) in EtOH (5 mL) and H₂O (1 mL) was added Fe (44.75 mg, 0.80 mmol) and NH₄Cl (214.33 mg, 4.01 mmol). The mixture was stirred at 65° C. for 1 h. The mixture was filtered and the filtrate was concentrated in vacuo to give a residue, which was purified (PM38) to afford compound 1.899 (120 mg, 0.315 mmol, 78.8% yield) as a light-yellow oil.

LCMS (AM6): rt=1.047 min, (319.2 [M+H]⁺), 83.8% purity.

### tert-butyl ((2-chloro-[1,1′-biphenyl]-4-yl)methyl)(3-((3-((1-(N,N-dimethylsulfamoyl)-6-(pyridin-4-yl)-1H-benzo[d][1,2,3]triazol-4-yl)amino)propyl)amino)-3-oxopropyl)carbamate 1.900

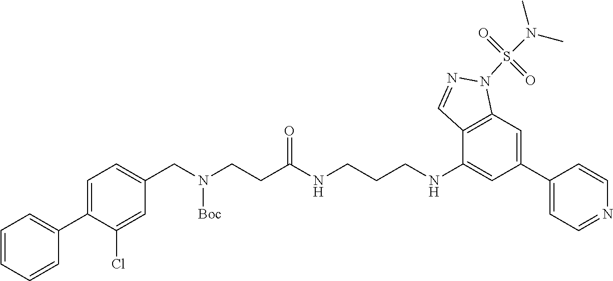 [SMILES: CN(C)S(=O)(=O)n1ncc2c(NCCCNC(=O)CCN(Cc3ccc(-c4ccccc4)c(Cl)c3)C(=O)OC(C)(C)C)cc(-c3ccncc3)cc21]

To a solution of compound 1.899 (110 mg, 0.289 mmol) in MeOH (5 mL) at 25° C. was added Intermediate H (193.25 mg, 0.434 mmol) and the reaction mixture stirred for 11 h. NaBH₃CN (181.96 mg, 2.90 mmol) was added and the mixture stirred for 1 h. The resultant mixture was concentrated in vacuo to give a residue, which was purified (PM111) to afford compound 1.900 (50 mg, 22.2% yield) as a yellow solid.

LCMS (AM3): rt=0.816 min, (747.2 [M+H]⁺), 96.1% purity.

### Synthesis of Intermediate 1.903

### (4-nitro-1-(tetrahydro-2H-pyran-2-yl)-1H-indazol-6-yl)boronic acid 1.901

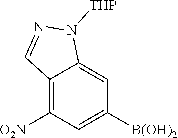

To a solution of 6-bromo-4-nitro-1-(tetrahydro-2H-pyran-2-yl)-1H-indazole (WO2019027960, 1 g, 3.07 mmol) in dioxane (10 mL) was added KOAc (601.83 mg, 6.13 mmol), 4,4,4′,4′,5,5,5′,5′-octamethyl-2,2′-bi(1,3,2-dioxaborolane) (778.60 mg, 3.07 mmol) and Pd(dppf)Cl₂ (224.35 mg, 0.31 mmol). The mixture was stirred at 90° C. for 25 h. The mixture was concentrated in vacuo to give a residue, which was purified (PM112) to afford compound 1.901 (0.62 g, 62.1% yield) as a light-yellow solid.

¹H NMR: (400 MHz, MeOH-d₄) δ: 8.65-8.54 (m, 3H), 6.06-6.00 (m, 1H), 3.94-3.89 (m, 1H), 3.83-3.76 (m, 1H), 2.48-2.42 (m, 1H), 2.08-2.02 (m, 2H), 1.79-1.75 (m, 1H), 1.64-1.64 (m, 2H) ppm.

### 6-(4-methyl-1H-imidazol-1-yl)-4-nitro-1-(tetrahydro-2H-pyran-2-yl)-1H-indazole 1.902

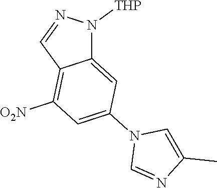

To a solution of compound 1.901 (570 mg, 1.75 mmol) in DMF (2 mL) was added TEA (708.63 mg, 7.00 mmol), 4-methyl-1H-imidazole (215.61 mg, 2.63 mmol), 4A molecular sieves (50 mg, 1.75 mmol) and Cu(OAc)₂ (381.59 mg, 2.10 mmol). The mixture was stirred at 65° C. for 12 h under O₂. The mixture was filtered and the filtrate was concentrated in vacuo to give a residue, which was purified (PM46) to afford compound 1.902 (200 mg, 30.9% yield) as a red solid.

LCMS (AM3): rt=0.746 min, (328.1 [M+H]⁺), 88.7% purity.

### 6-(4-methyl-1H-imidazol-1-yl)-1-(tetrahydro-2H-pyran-2-yl)-1H-indazol-4-amine 1.903

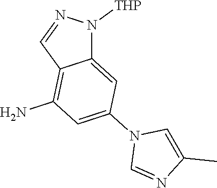

To a solution of NiCl₂·6H₂O (61.19 mg, 0.257 mmol) in MeOH (10 mL) was added NaBH₄ (9.74 mg, 0.257 mmol). After 5 minutes, compound 1.902 (190 mg, 0.514 mmol) and NaBH₄ (58.43 mg, 1.54 mmol) was added. The mixture was stirred at 0° C. for 25 minutes. The mixture was filtered and the filtrate was concentrated in vacuo to give a residue, which was purified (PM113) to afford compound 1.903 (180 mg, 92.8% yield) as a red solid.

LCMS (AM3): rt=0.235 min, (297.9 [M+H]⁺), 79.0% purity.

### Synthesis of Intermediate 1.910

### tert-butyl (4-(2-(5-bromo-3-fluoro-2-formylphenoxy)ethoxy)butyl)carbamate 1.906

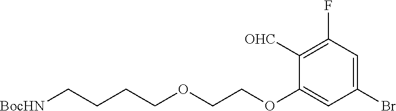 [SMILES: CC(C)(C)OC(=O)NCCCCOCCOc1cc(Br)cc(F)c1C=O]

To a solution of 4-bromo-2-fluoro-6-hydroxybenzaldehyde (CAS 1427438-90-1, 12 g, 54.79 mmol) in DMF (150 mL) was added K₂CO₃ (18.93 g, 136.98 mmol) and compound 1.372 (22.57 g, 65.75 mmol). The mixture was stirred at 80° C. for 12 h. The mixture was extracted with EA (1000 mL) and washed with H₂O (200 mL×3). The organic phase was dried (Na₂SO₄), filtered and concentrated in vacuo to give a residue, which was purified (PM47) to afford compound 1.906 (43 g, 89.3% yield) as a light-yellow oil.

LCMS (AM3): rt=0.965 min, (455.9 [M+Na]⁺), 88.5% purity.

### tert-butyl (4-(2-((6-bromo-1H-indazol-4-yl)oxy)ethoxy)butyl)carbamate 1.907

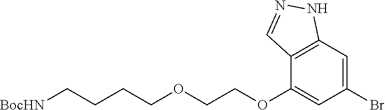 [SMILES: CC(C)(C)OC(=O)NCCCCOCCOc1cc(Br)cc2[nH]ncc12]

To a solution of compound 1.906 (43 g, 87.62 mmol) in ethylene glycol (500 mL) was added NH₂NH₂·H₂O (76.22 g, 1.52 mol). The mixture was stirred at 100° C. for 12 h. The mixture was concentrated in vacuo to give a residue, which was purified (PM43) to afford compound 1.907 (27 g, 67.0% yield) as a light-yellow oil.

LCMS (AM3): rt=0.934 min, (428.1 [M+H]⁺), 96.5% purity.

### tert-butyl (4-(2-((6-bromo-1-(tetrahydro-2H-pyran-2-yl)-1H-indazol-4-yl)oxy)ethoxy)butyl)carbamate 1.908

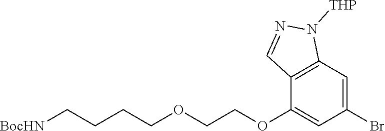

To a solution of compound 1.907 (27 g, 63.04 mmol) in THF (100 mL) was added TsOH·H₂O (1.20 g, 6.30 mmol) and DHP (53.02 g, 630.37 mmol). The mixture was stirred at 60° C. for 12 h. The mixture was concentrated in vacuo to give a residue, which was purified (PM115) to afford compound 1.908 (9.8 g, 50.9% yield) as a yellow oil.

LCMS (AM3): rt=0.882 min, (512.1 [M+H]⁺), 84.0% purity.

### tert-butyl (4-(2-((6-(isoxazol-4-yl)-1-(tetrahydro-2H-pyran-2-yl)-1H-indazol-4-yl)oxy)ethoxy)butyl)carbamate 1.909

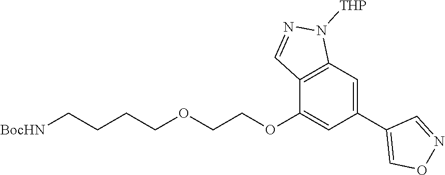

To a solution of compound 1.908 (9.8 g, 16.06 mmol) in H₂O (48 mL) and DMF (100 mL) was added KF (2.80 g, 48.19 mmol), 4-(tetramethyl-1,3,2-dioxaborolan-2-yl)-1,2-oxazole (CAS 928664-98-6, 3.76 g, 19.28 mmol) and Pd(dppf)Cl₂ (1.18 g, 1.61 mmol). The mixture was stirred at 60° C. for 2 h under N₂. The mixture was concentrated in vacuo to give a residue, which was purified (PM43) to afford compound 1.909 (4.4 g, 54.7% yield) as a light-yellow oil.

LCMS (AM3): rt=0.985 min, (501.2 [M+H]⁺), 100% purity.

### 4-(2-((6-(isoxazol-4-yl)-1H-indazol-4-yl)oxy)ethoxy)butan-1-amine 1.910

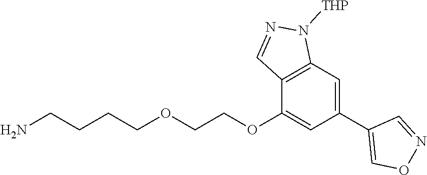

To a solution of compound 1.909 (4.4 g, 8.79 mmol) in dioxane (20 mL) was added HCl/dioxane (4 M, 50 mL) at 0° C. The mixture was stirred at 25° C. for 2 h, then concentrated in vacuo to give a residue, which was purified (PM116) to afford compound 1.910 (2.7 g, 82.4% yield, HCl salt) as a red solid.

LCMS (AM3): rt=0.668 min, (317.0 [M+H]⁺), 84.9% purity.

### Synthesis of Intermediate 1.913

### tert-butyl (4-(2-((1-(tetrahydro-2H-pyran-2-yl)-6-(4,4,5,5-tetramethyl-1,3,2-dioxaborolan-2-yl)-1H-indazol-4-yl)oxy)ethoxy)butyl)carbamate 1.911

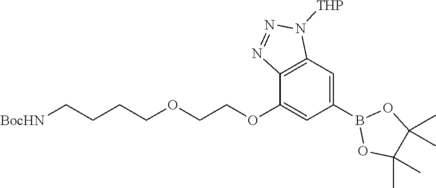

To a solution of compound 1.908 (6.5 g, 10.78 mmol) in dioxane (50 mL) was added 4,4,5,5-tetramethyl-2-(4,4,5,5-tetramethyl-1,3,2-dioxaborolan-2-yl)-1,3,2-dioxaborolane (3.29 g, 12.94 mmol), KOAc (2.12 g, 21.56 mmol) and Pd(dppf)Cl₂ (788.92 mg, 1.08 mmol). The mixture was stirred at 85° C. for 12 h under N₂. The mixture was concentrated in vacuo to give a residue, which was purified (PM40) to afford compound 1.911 (6.5 g, 92.6% yield) as a yellow oil.

LCMS (AM3): rt=0.908, 0.988 min, (560.4 [M+H]⁺), 80.2% purity.

### tert-butyl (4-(2-((6-(pyridazin-4-yl)-1-(tetrahydro-2H-pyran-2-yl)-1H-indazol-4-yl)oxy)ethoxy)butyl)carbamate 1.912

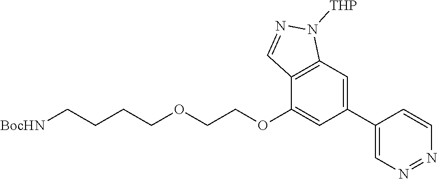

To a solution of compound 1.911 (6.50 g, 10.00 mmol) in dioxane (100 mL) and H₂O (10 mL) under N₂ was added Pd(dppf)Cl₂ (664.92 mg, 0.91 mmol), 4-bromopyridazine (2.18 g, 9.09 mmol, HBr salt) and K₂CO₃ (3.77 g, 27.26 mmol). The mixture was stirred at 80° C. for 12 h. The mixture was concentrated in vacuo to give a crude residue, which was purified (PM117) to afford compound 1.912 (2.9 g, 44.3% yield) as a yellow solid.

LCMS (AM3): rt=0.889 min, (512.1 [M+H]⁺), 71.1% purity.

### 4-(2-((6-(pyridazin-4-yl)-1H-indazol-4-yl)oxy)ethoxy)butan-1-amine 1.913

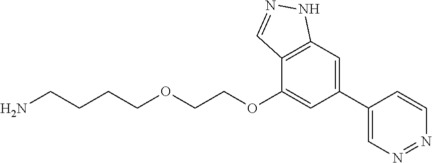 [SMILES: NCCCCOCCOc1cc(-c2ccnnc2)cc2[nH]ncc12]

To a solution of compound 1.912 (2.85 g, 3.96 mmol) in MeOH (10 mL) was added HCl/MeOH (4 M, 50 mL). The mixture was stirred at 25° C. for 2 h. The mixture was concentrated in vacuo to give a residue, which was recrystallized with EA (100 mL) at 25° C. to afford compound 1.913 (1.8 g, 96.6% yield, HCl salt) as a yellow solid.

LCMS (AM3): rt=0.611 min, (328.0 [M+H]⁺), 85.0% purity.

### Synthesis of Intermediate 1.915

### (4-((3-(3-((tert-butoxycarbonyl)((2-chloro-[1,1′-biphenyl]-4-yl)methyl)amino)propanamido)propyl)amino)-1-(tetrahydro-2H-pyran-2-yl)-1H-indazol-6-yl)boronic acid 1.914

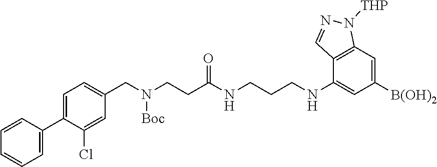

To a solution of compound 1.382 (1.45 g, 2.00 mmol) in dioxane (10 mL) under N₂ was added KOAc (392.51 mg, 4.00 mmol), Pd(dppf)Cl₂ (146.32 mg, 0.2 mmol) and 4,4,5,5-tetramethyl-2-(4,4,5,5-tetramethyl-1,3,2-dioxaborolan-2-yl)-1,3,2-dioxaborolane (609.36 mg, 2.40 mmol). The mixture was stirred at 85° C. for 12 h. The solid was filtered and the filtrate was concentrated in vacuo to give a residue, which was purified (PM118) to afford compound 1.914 (900 mg, 1.24 mmol, 61.77% yield) as a dark brown solid.

LCMS (AM3): rt=1.004 min, (690.4 [M+H]⁺), 94.7% purity.

### 4-(4-((3-(3-((tert-butoxycarbonyl)((2-chloro-[1,1′-biphenyl]-4-yl)methyl)amino)propanamido)propyl)amino)-1-(tetrahydro-2H-pyran-2-yl)-1H-indazol-6-yl)pyridine 1-oxide 1.915

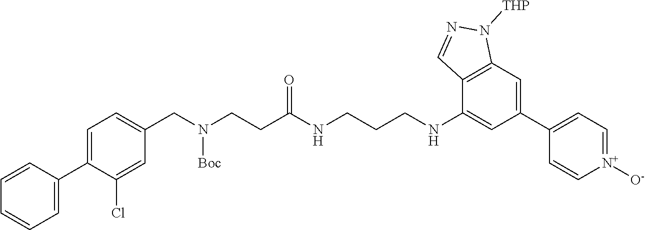

To a solution of compound 1.914 (200 mg, 0.29 mmol) in H₂O (0.5 mL) and DME (5 mL) under N₂ was added K₂CO₃ (80.12 mg, 0.58 mmol), 4-chloropyridine 1-oxide (45.06 mg, 0.32 mmol) and Pd(PPh₃)₄ (33.49 mg, 0.029 mmol). The mixture was stirred at 80° C. for 12 h. The resultant mixture was then concentrated in vacuo to give a residue, which was purified (PM90) to afford compound 1.915 (0.15 g, 61.6% yield) as a yellow solid.

LCMS (AM3): rt=1.023 min, (739.4 [M+H]⁺), 88.4% purity.

### Synthesis of Intermediate 1.918

### 4-bromo-N, N, 2-trimethyl-1H-imidazole-1-sulfonamide 1.917

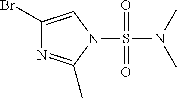

To a solution of 4-Bromo-2-methyl-1H-imidazole, (CAS 16265-11-5, 350 mg, 2.17 mmol) in DCM (10 mL) at 25° C. was added TEA (879.91 mg, 8.70 mmol) and dimethylsulfamoyl chloride (468.24 mg, 3.26 mmol) and the mixture stirred for 12 h. The mixture was concentrated in vacuo to give a residue, which was purified (PM64) to afford compound 1.917 (200 mg, 31.9% yield) as a light-yellow oil.

LCMS (AM1): rt=0.698 min, (270.2 [M+H]⁺), 93.4% purity.

### tert-butyl ((2-chloro-[1,1′-biphenyl]-4-yl)methyl)(3-((3-((6-(1-(N,N-dimethylsulfamoyl)-2-methyl-1H-imidazol-4-yl)-1-(tetrahydro-2H-pyran-2-yl)-1H-indazol-4-yl)amino)propyl)amino)-3-oxopropyl)carbamate 1.918

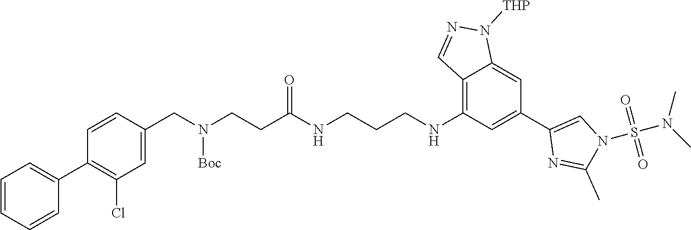

To a solution of compound 1.914 (250 mg, 0.36 mmol) in H₂O (0.5 mL) and DME under N₂ (5 mL) was added K₂CO₃ (125.18 mg, 0.91 mmol), compound 1.917 (145.72 mg, 0.54 mmol) and Pd(PPh₃)₄(41.87 mg, 0.036 mmol). The mixture was stirred at 80° C. for 12 hrs. The mixture was concentrated in vacuo to give a residue, which was purified (PM119) to afford compound 1.918 (100 mg, 32.7% yield) as a light-yellow oil.

LCMS (AM1): rt=0.960 min, (833.0 [M+H]⁺), 98.9% purity.

### Synthesis of Intermediate 1.921

### tert-butyl(4-(2-((1-(tetrahydro-2H-pyran-2-yl)-6-(4,4,5,5-tetramethyl-1,3,2-dioxaborolan-2-yl)-1H-indazol-4-yl)amino)ethoxy)butyl)carbamate 1.919

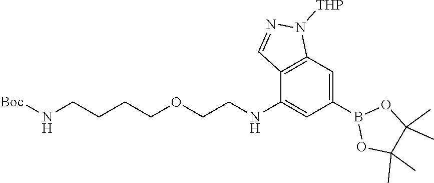

To a solution of compound 1.893 (2.8 g, 5.47 mmol) in dioxane (40 mL) was added 4,4,4′,4′,5,5,5′,5′-octamethyl-2,2′-bi(1,3,2-dioxaborolane) (2.09 g, 8.21 mmol), Pd(dppf)Cl₂ (400.58 mg, 547.46 μmol) and KOAc (1.07 g, 10.95 mmol). The reaction mixture was stirred at 80° C. for 12 h. The reaction mixture was concentrated in vacuo to give a residue, which was purified (PM2) to afford compound 1.919 (3.2 g, crude) as a yellow oil.

LCMS (AM3): rt=1.095 min, (559.4 [M+H]⁺), 75.9% purity.

### tert-butyl (4-(2-((6-(pyridazin-4-yl)-1-(tetrahydro-2H-pyran-2-yl)-1H-indazol-4-yl)amino)ethoxy)butyl)carbamate 1.920

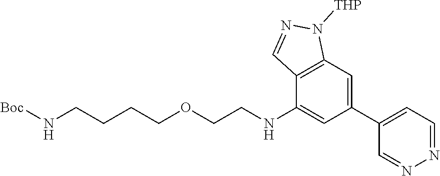

To a solution of compound 1.919 (3.1 g, 5.55 mmol) in dioxane (15 mL) and H₂O (1.5 mL) was added K₂CO₃ (1.53 g, 11.10 mmol), 4-bromopyridazine (1.63 g, 8.33 mmol, HCl salt) and Pd(dppf)Cl₂ (406.13 mg, 555.04 μmol). The reaction mixture was stirred at 80° C. for 12 h under N₂. The reaction mixture was filtered and the filtrate was concentrated in vacuo to give a residue, which was purified (PM2) to afford compound 1.920 (2.6 g, 5.09 mmol, 91.74% yield) as a yellow oil.

LCMS (AM3): rt=0.904 min, (511.1 [M+H]⁺), 100% purity.

### N-(2-(4-aminobutoxy)ethyl)-6-(pyridazin-4-yl)-1H-indazol-4-amine 1.921

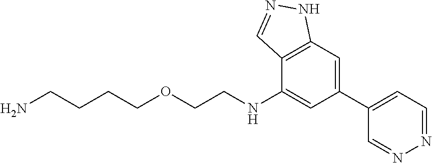 [SMILES: NCCCCOCCNc1cc(-c2ccnnc2)cc2[nH]ncc12]

To a solution of compound 1.920 (2.6 g, 5.09 mmol) in dioxane (10 mL) was added HCl/dioxane (4 M, 10 mL) slowly. The reaction mixture was stirred at 20° C. for 0.5 h. The reaction mixture was concentrated in vacuo to afford compound 1.921 (2.2 g, crude, HCl salt) as a red solid.

LCMS (AM3): rt=0.761 min, (326.9 [M+H]⁺), 93.4% purity.

### Synthesis of Intermediate 1.923

### (4-amino-1-(tetrahydro-2H-pyran-2-yl)-1H-indazol-6-yl)boronic acid 1.922

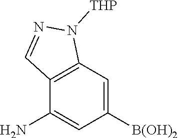

A solution of compound 1.901 (3.5 g, 9.02 mmol) in MeOH (10 mL) was added Pd/C (500 mg, 9.02 mmol, 10% purity) under N₂. The suspension was degassed and purged with H₂ (×3). The mixture was stirred at 25° C. for 12 h under H₂ (15 psi). The mixture was filtered and concentrated in vacuo to afford compound 1.922 (3 g, crude) as a brown oil, which was used for the next step without further purification.

LCMS (AM3): rt=0.508 min, (260.4 [M+H]⁺), 42.07% purity

### 6-(pyridazin-4-yl)-1-(tetrahydro-2H-pyran-2-yl)-1H-indazol-4-amine 1.923

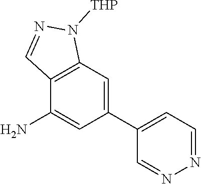

To a mixture of compound 1.922 (200 mg, 766.04 μmol) and 4-bromopyridazine (240.03 mg, 919.24 μmol) in dioxane (2 mL) and H₂O (0.2 mL) was added K₂CO₃ (211.75 mg, 1.53 mmol) and Pd(dppf)Cl₂·CH₂Cl₂ (62.56 mg, 76.60 μmol) in one portion under N₂. The mixture was stirred at 90° C. for 12 h. The mixture was filtered and the filtrate was concentrated in vacuo to give a residue, which was purified (PM38) and then by prep-TLC (PM2) to afford compound 1.923 (200 mg, 457.90 μmol, 59.77% yield) as a yellow oil.

LCMS (AM3): rt=0.873 min, (398.6 [M+H]⁺), 86.0% purity.

### Synthesis of Intermediate 1.927

### 4-bromo-1-(tetrahydro-2H-pyran-2-yl)-1H-pyrazole-3-carbonitrile 1.926

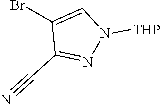

To a mixture of 4-Bromo-1H-pyrazole-5-carbonitrile (CAS 288246-16-2, 500 mg, 2.91 mmol) and DHP (366.82 mg, 4.36 mmol) in THF (10 mL) was added TsOH·H₂O (276.51 mg, 1.45 mmol) under N₂. The mixture was stirred at 60° C. for 12 h. The mixture was filtered and the filtrate was concentrated in vacuo to give a residue, which was purified (PM49) to afford compound 1.926 (600 mg, 2.34 mmol, 80.59% yield) as a colourless oil.

¹H NMR (400 MHz, MeOH-d₄) δ 8.13 (s, 1H), 5.50-5.47 (m, 1H), 4.00-3.97 (m, 1H), 3.73-3.70 (m, 1H), 2.09-2.06 (m, 2H), 2.04-2.03 (m, 1H), 1.67-1.65 (m, 2H), 1.63-1.62 (m, 1H) ppm.

### 4-(4-amino-1-(tetrahydro-2H-pyran-2-yl)-1H-indazol-6-yl)-1-(tetrahydro-2H-pyran-2-yl)-1H-pyrazole-3-carbonitrile 1.927

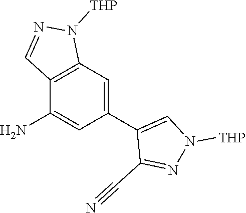

To a mixture of compound 1.926 (200 mg, 766.04 μmol) and compound 1.922 (235.42 mg, 919.24 μmol) in dioxane (2 mL) and H₂O (0.2 mL) was added K₂CO₃ (211.75 mg, 1.53 mmol) and Pd(dppf)Cl₂·CH₂Cl₂ (62.56 mg, 76.60 μmol) in one portion under N₂. The mixture was stirred at 90° C. for 12 h. The mixture was filtered and the filtrate was concentrated in vacuo to give a residue, which was purified (PM38) to afford compound 1.927 (170 mg, 350.87 μmol, 45.80% yield) as a yellow oil.

LCMS (AM3): rt=0.913 min, (393.5 [M+H]⁺), 81.3% purity.

### Synthesis of Intermediate 1.931

### 4-bromo-2-((2-(trimethylsilyl)ethoxy)methyl)-2H-1,2,3-triazole 1.929

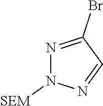 [SMILES: C[Si](C)(C)CCOCn1ncc(Br)n1]

To a mixture of 5-Bromo-1H-1,2,3-triazole (CAS 40964-56-5, 450 mg, 3.04 mmol) in THF (5 mL) was added NaH (182.46 mg, 4.56 mmol, 60% purity) at 0° C. and stirred for 0.5 h, then SEM-CI (608.47 mg, 3.65 mmol) was added and the mixture was stirred at 30° C. for 0.5 h. The mixture was quenched with water (20 mL) and the aqueous phase was extracted with EA (20 mL×2). The combined organic phase was washed (brine, 30 mL), dried (Na₂SO₄), filtered and concentrated in vacuo to give a residue, which was purified (PM50) to afford compound 1.929 (550 mg, 1.98 mmol, 65.00% yield) as a colourless oil.

¹H NMR (400 MHz, CHCl₃-d) δ 7.72 (d, J=17.6 Hz, 1H), 5.69 (d, J=15.6 Hz, 2H), 3.69-3.53 (m, 2H), 0.95-0.89 (m, 2H), −0.01 (d, J=4.4 Hz, 9H) ppm.

### tert-butyl((2-chloro-[1,1′-biphenyl]-4-yl)methyl)(3-oxo-3-((3-((1-(tetrahydro-2H-pyran-2-yl)-6-(2-((2-(trimethylsilyl)ethoxy)methyl)-2H-1,2,3-triazol-4-yl)-1H-indazol-4-yl)amino)propyl)aminopropyl)carbamate 1.930

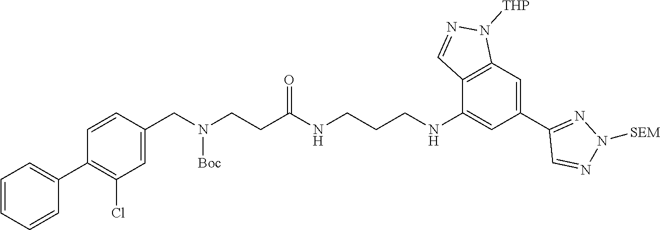

To a mixture of compound 1.929 (120.96 mg, 434.76 μmol) and compound 1.914 (150 mg, 217.38 μmol) in DME (3 mL) and H₂O (0.3 mL) was added K₂CO₃ (60.09 mg, 434.76 mol) and Pd(PPh₃)₄(25.12 mg, 21.74 μmol). The mixture was stirred at 90° C. for 16 h under N₂. The mixture was concentrated in vacuo to give a residue, which was purified by prep-TLC (EA) to afford compound 1.930 (110 mg, 130.40 μmol, 59.99% yield) as a purple oil.

LCMS (AM3): rt=1.165 min, (843.4 [M+H]⁺), 59% purity.

### tert-butyl((2-chloro-[1,1′-biphenyl]-4-yl)methyl)(3-oxo-3-((3-((1-(tetrahydro-2H-pyran-2-yl)-6-(2H-1,2,3-triazol-4-yl)-1H-indazol-4-yl)amino)propyl)amino)propyl)carbamate 1.931

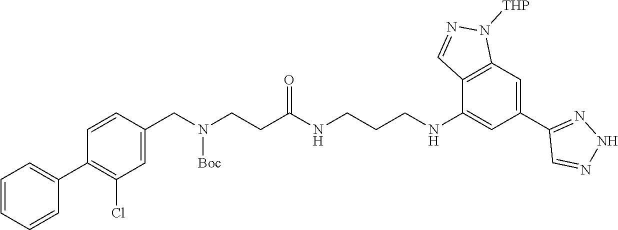

A mixture of compound 1.930 (160 mg, 189.68 μmol) and TBAF (1 M, 5 mL) was stirred at 70° C. for 1 h. The mixture was concentrated in vacuo to give a residue, which was purified (PM121) to afford compound 1.931 (90 mg, 126.18 μmol, 66.52% yield) as a yellow solid.

LCMS (AM3): rt=1.080 min, (713.3 [M+H]⁺),93% purity.

### Synthesis of Intermediate 1.941

### 2-(4-((3,5-difluoro-4-(trifluoromethoxy)benzyl)amino)butoxy)ethan-1-ol 1.939

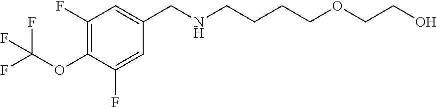 [SMILES: OCCOCCCCNCc1cc(F)c(OC(F)(F)F)c(F)c1]

To a mixture of 3,5-Difluoro-4-(trifluoromethoxy)benzaldehyde (WO2022185041, 12.8 g, 42.46 mmol, 75% purity) and 2-(4-aminobutoxy)ethan-1-ol (CAS 1019567-93-1, 8.64 g, 50.93 mmol, HCl salt) in MeOH (120 mL) was added DIPEA (7.13 g, 55.20 mmol) at 20° C. and the mixture stirred for 12 h, NaBH₃CN (8.00 g, 127.38 mmol) was added portionwise. The resulting mixture was stirred for 3 h. The reaction mixture was diluted with H₂O (50 mL) and filtered. The filtrate was concentrated in vacuo to give a residue, which was purified (PM241) to afford compound 1.939 (11.9 g, 24.98 mmol, 58.83% yield, 96% purity, TFA salt) as a yellow oil.

LCMS (AM16): rt=0.678 min, (344.2 [M+H]⁺), 96.2% purity.

¹H NMR (400 MHz, MeOH-d₄) δ 7.44-7.36 (m, 2H), 4.26 (s, 2H), 3.70-3.66 (m, 2H), 3.57-3.52 (m, 4H), 3.12 (t, J=7.2 Hz, 2H), 1.89-1.79 (m, 2H), 1.77-1.67 (m, 2H) ppm.

### tert-butyl (3,5-difluoro-4-(trifluoromethoxy)benzyl)(4-(2-hydroxyethoxy)butyl)carbamate 1.940

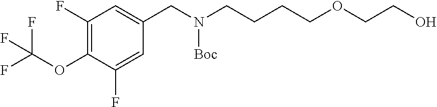 [SMILES: CC(C)(C)OC(=O)N(CCCCOCCO)Cc1cc(F)c(OC(F)(F)F)c(F)c1]

To a solution of compound 1.939 (11.9 g, 26.02 mmol, TFA salt) in THF (150 mL) and H₂O (50 mL) was added NaHCO₃ (6.56 g, 78.06 mmol) followed by Boc₂O (6.81 g, 31.23 mmol). The mixture was stirred at 2° C. for 12 h. The reaction mixture was poured onto H₂O (200 mL) and extracted with EA (150 mL×3). The combined organic phase was washed (brine, 100 mL×2), dried (Na₂SO₄), filtered and concentrated in vacuo to give a residue, which was purified (PM45) to afford compound 1.940 (9.6 g, 20.18 mmol, 77.54% yield, 93.2% purity) as a colourless oil.

LCMS (AM11): rt=0.492 min, (466.0 [M+Na]⁺), 93.2% purity.

¹H NMR (400 MHz, CHCl₃-d) δ 6.93-6.84 (m, 2H), 4.39 (s, 2H), 3.78-3.70 (m, 2H), 3.55-3.45 (m, 4H), 3.32-3.16 (m, 2H), 1.58-1.34 (m, 13H) ppm.

### tert-butyl (3,5-difluoro-4-(trifluoromethoxy)benzyl)(4-(2-oxoethoxy)butyl)carbamate 1.941

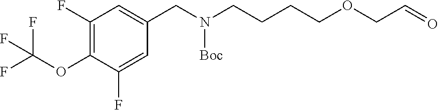 [SMILES: CC(C)(C)OC(=O)N(CCCCOCC=O)Cc1cc(F)c(OC(F)(F)F)c(F)c1]

To a solution of oxalyl dichloride (2.86 g, 22.55 mmol) in DCM (40 mL) was added a solution of DMSO (2.64 g, 33.83 mmol) in DCM (10 mL) at −70° C. under N₂. The mixture was cooled to −70° C. for 0.5 hr, a solution of compound 1.940 (5 g, 11.28 mmol) in DCM (30 mL) was added dropwise at −70° C., after 0.5 hr TEA (6.85 g, 67.66 mmol) was added to the mixture at −70° C., the mixture was warmed to 20° C. and stirred for 10 minutes. The reaction mixture was filtered and the filtrate was purified (PM43) to afford compound 1.941 (4.95 g, 11.21 mmol, 99.45% yield) as a yellow oil.

¹H NMR (400 MHz, DMSO-d₆) δ 9.57 (s, 1H), 7.25-7.16 (m, 2H), 4.40 (s, 2H), 3.48-3.35 (m, 2H), 3.25-3.15 (m, 2H), 1.55-1.44 (m, 4H), 1.43-1.27 (m, 9H) ppm.

### Synthesis of Intermediate 1.945

### 4-nitro-1-(tetrahydro-2H-pyran-2-yl)-6-(4,4,5,5-tetramethyl-1,3,2-dioxaborolan-2-yl)-1H-indazole 1.943

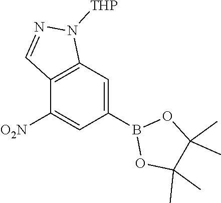

To a solution of 6-bromo-4-nitro-1-(tetrahydro-2H-pyran-2-yl)-1H-indazole (WO2019027960, 3 g, 9.20 mmol) in dioxane (45 mL) was added 4,4,4′,4′,5,5,5′,5′-octamethyl-2,2′-bi(1,3,2-dioxaborolane) (2.80 g, 11.04 mmol) followed by KOAc (1.81 g, 18.40 mmol) and Pd(dppf)Cl₂ (673.05 mg, 919.83 μmol). The mixture was degassed and purged with N₂ (×3), then the mixture was stirred at 90° C. for 3 h. The reaction mixture was cooled to 20° C., diluted with DCM (100 mL) and filtered. The filter cake was washed with DCM (60 mL×2) and the filtrate was concentrated in vacuo to give a residue, which was purified (PM36) to afford compound 1.943 (3.09 g, 8.28 mmol, 90.08% yield) as an off-white solid.

¹H NMR (400 MHz, CHCl₃-d) δ 8.66 (s, 1H), 8.55 (s, 1H), 8.37 (s, 1H), 5.87 (dd, J=9.2, 2.8 Hz, 1H), 4.03-4.00 (m, 1H), 3.86-3.75 (m, 1H), 2.65-2.53 (m, 1H), 2.3-2.16 (m, 1H), 2.15-2.05 (m, 1H), 1.79-1.73 (m, 3H), 1.40 (s, 12H) ppm.

### 4-(4-nitro-1-(tetrahydro-2H-pyran-2-yl)-1H-indazol-6-yl)pyridin-2(1H)-one 1.944

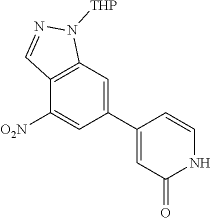

To a mixture of compound 1.943 (2 g, 5.36 mmol) in dioxane (30 mL) and H₂O (3 mL) was added 4-bromopyridin-2(1H)-one (1.12 g, 6.43 mmol) and K₂CO₃ (1.48 g, 10.72 mmol) followed by Pd₂(dba)₃ (490.72 mg, 535.89 μmol) at 20° C. The mixture was degassed and purged with N₂ (×3), then the mixture stirred at 90° C. for 12 h. The mixture was filtered and washed with EA (10 mL×3), then the filtrate was concentrated in vacuo to give a residue, which was triturated with EA (20 mL) at 20° C. for 5 minutes, then the filter cake was concentrated in vacuo to afford compound 1.944 (900 mg, 2.43 mmol, 45.40% yield) as a yellow solid.

LCMS (AM11): rt=0.363 min, (341.0 [M+H]⁺), 92.07% purity.

¹H NMR (400 MHz, DMSO-d₆) δ 8.67 (s, 1H), 8.60 (s, 1H), 8.38 (d, J=1.6 Hz, 1H), 7.60 (d, J=6.8 Hz, 1H), 6.87 (d, J=1.6 Hz, 1H), 6.71 (dd, J=5.6, 1.6 Hz, 1H), 6.24-6.18 (m, 1H), 3.89-3.82 (m, 2H), 2.07 (s, 2H), 2.06-2.00 (m, 2H), 1.63-1.58 (m, 2H) ppm.

### 4-(4-amino-1-(tetrahydro-2H-pyran-2-yl)-1H-indazol-6-yl)pyridin-2(1H)-one 1.945

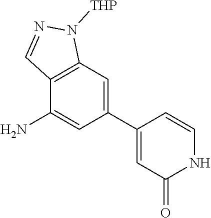

To a mixture of compound 1.944 (800 mg, 2.35 mmol) in EtOH (20 mL) and H₂O (5 mL) was added NH₄Cl (1.26 g, 23.51 mmol) and Fe (1.05 g, 18.81 mmol) at 20° C. The mixture was degassed and purged with N₂ (×3) and the mixture heated to 70° C. and stirred for 12 h. The mixture was concentrated in vacuo to give a residue, which was purified (PM136) to afford compound 1.945 (280 mg, 902.21 μmol, 34.12% yield) as a yellow gum.

¹H NMR (400 MHz, DMSO-d₆) δ 8.15 (s, 1H), 7.43 (d, J=6.8 Hz, 1H), 7.09 (s, 1H), 6.53 (d, J=1.6 Hz, 1H), 6.49-6.46 (m, 1H), 6.46-6.44 (m, 1H), 5.99 (s, 2H), 5.81 (dd, J=9.6, 2.4 Hz, 1H), 3.91-3.84 (m, 1H), 3.80-3.70 (m, 1H), 3.40-3.35 (m, 2H), 2.08-2.00 (m, 1H), 1.95-1.87 (m, 1H), 1.62-1.52 (m, 2H) ppm.

### Synthesis of Intermediate 1.951

### 3-((3-chloro-4-(trifluoromethoxy)benzyl)amino)propanoic acid 1.947

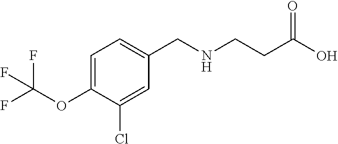 [SMILES: O=C(O)CCNCc1ccc(OC(F)(F)F)c(Cl)c1]

To a mixture of 3-chloro-4-(trifluoromethoxy)benzaldehyde (CAS 83279-39-4, 12.5 g, 55.66 mmol) in MeOH (150 mL) was added 3-aminopropanoic acid (14.88 g, 166.99 mmol). The mixture stirred at 20° C. for 12 h, then NaBH₃CN (7.00 g, 111.33 mmol) was added in portions at 0° C. The mixture was stirred at 20° C. for 2 h. The reaction mixture was filtered and the filter cake washed with MeOH (30 mL×3). The filtrate was concentrated in vacuo to give a residue, which was purified (PM137) to afford compound 1.947 (18 g, 43.20 mmol, 68.68% yield, TFA salt) as a yellow oil.

LCMS (AM11): rt=0.310 min, (298.0 [M+H]⁺), 98.89% purity.

### 3-((tert-butoxycarbonyl)(3-chloro-4-(trifluoromethoxy)benzyl)amino)propanoic acid 1.948

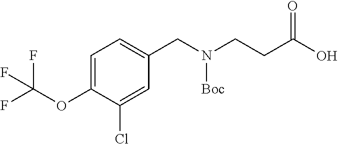 [SMILES: CC(C)(C)OC(=O)N(CCC(=O)O)Cc1ccc(OC(F)(F)F)c(Cl)c1]

To a solution of compound 1.947 (18 g, 43.72 mmol, TFA salt) in THF (150 mL), MeOH (70 mL) and H₂O (70 mL) was added NaHCO₃ (14.69 g, 174.89 mmol) followed by Boc₂O (9.54 g, 43.72 mmol) at 20° C. The mixture was stirred at 20° C. for 12 h. The reaction mixture was concentrated in vacuo, diluted with water (150 mL), acidified to pH=5 by HCl (aq.) (1 M) and extracted with EA (150 mL×3). The combined organic phase was washed (brine, 150 mL×2), dried (Na₂SO₄), filtered and concentrated in vacuo to afford compound 1.948 (16 g, 40.22 mmol, 92.00% yield) as a yellow oil.

LCMS (AM12): rt=0.588 min, (420.2 [M+Na]⁺), 96.89% purity.

¹H NMR (400 MHz, CHCl₃-d) δ 7.34 (s, 1H), 7.31-7.27 (m, 1H), 7.19-7.11 (m, 1H), 4.45 (s, 2H), 3.49 (br s, 2H), 2.64 (br s, 2H), 1.48 (s, 9H) ppm.

### tert-butyl (3-chloro-4-(trifluoromethoxy)benzyl)(3-((3,3-diethoxypropyl)amino)-3-oxopropyl)carbamate 1.949

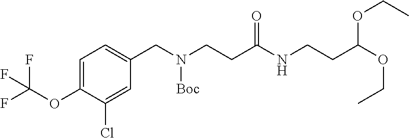 [SMILES: CCOC(CCNC(=O)CCN(Cc1ccc(OC(F)(F)F)c(Cl)c1)C(=O)OC(C)(C)C)OCC]

To a solution of compound 1.948 (8 g, 17.36 mmol, 86.3% purity) and 3,3-diethoxypropan-1-amine (2.81 g, 19.09 mmol) in THF (120 mL) was added DIPEA (8.97 g, 69.43 mmol) followed by T₃P (12.15 g, 19.09 mmol, 50% purity) dropwise at 0° C. The mixture was stirred at 20° C. for 12 h. The reaction mixture was poured into H₂O (200 mL) and extracted with EA (150 mL×3). The combined organic phase was washed by brine (150 mL×2), dried (Na₂SO₄), filtered and concentrated in vacuo to give a residue, which was purified (PM42) to afford compound 1.949 (7.1 g, 12.50 mmol, 72.04% yield) as a yellow oil.

¹H NMR (CHCl₃-d, 400 MHz) δ 7.35 (s, 1H), 7.29-7.09 (m, 2H), 6.43-6.10 (m, 1H), 4.56 (t, J=5.2 Hz, 1H), 4.43 (s, 2H), 3.63-3.73 (m, 2H), 3.55-3.48 (m, 4H), 3.37-3.32 (m, 2H), 2.51-2.30 (m, 2H), 1.87-1.78 (m, 4H), 1.45 (s, 9H), 1.22 (t, J=7.2 Hz, 6H) ppm.

### tert-butyl (3-chloro-4-(trifluoromethoxy)benzyl)(3-oxo-3-((3-oxopropyl)amino)propyl)carbamate 1.950

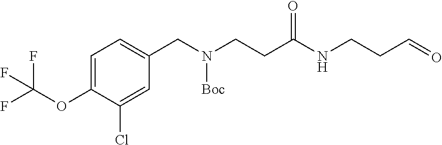 [SMILES: CC(C)(C)OC(=O)N(CCC(=O)NCCC=O)Cc1ccc(OC(F)(F)F)c(Cl)c1]

A one neck flask charged with compound 1.949 (2 g, 3.80 mmol) was cooled to 0° C. To this cooled mixture was added AcOH (20 mL) and H₂O (4 mL). The mixture was stirred at 20° C. for 1 h. The reaction mixture was added into sat. NaHCO₃ (aq.) (120 mL) at 0° C. and extracted with EA (60 mL×3). The combined organic phase was washed by brine (50 mL×2), dried (Na₂SO₄), filtered and concentrated in vacuo to afford compound 1.950 (1.4 g, 3.09 mmol, 81.46% yield) as a yellow oil.

¹H NMR (400 MHz, DMSO-d₆) δ 9.62 (s, 1H), 8.18-7.99 (m, 1H), 7.60-7.50 (m, 1H), 7.47 (s, 1H), 7.29 (d, J=7.6 Hz, 1H), 4.37 (s, 2H), 3.49-3.35 (m, 2H), 3.32-3.25 (m, 2H), 2.64-2.53 (m, 2H), 2.39-2.24 (m, 2H), 1.50-1.25 (m, 9H) ppm.

### tert-butyl 3-chloro-4-(trifluoromethoxy)benzyl(3-oxo-3-((3-((6-(2-oxo-1,2-dihydropyridin-4-yl)-1-(tetrahydro-2H-pyran-2-yl)-1H-indazol-4-yl)amino)propyl)amino) propyl)carbamate 1.951

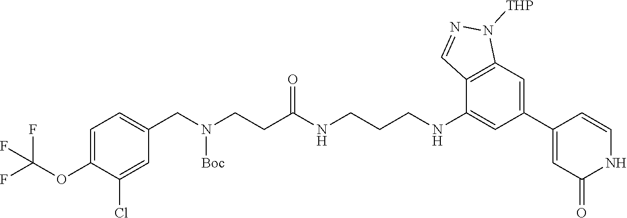

To a solution of compound 1.950 (132.65 mg, 292.92 μmol) in MeOH (2 mL) was added compound 1.945 (100 mg, 322.22 μmol). The reaction was stirred at 20° C. for 12 h, then NaBH₃CN (55.22 mg, 878.77 μmol) added and the reaction stirred for 20° C. for 2 h. The mixture was concentrated in vacuo to give a residue, which was purified (PM138) to afford compound 1.951 (48 mg, 64.24 μmol, 21.93% yield) as a yellow solid.

LCMS (AM12): rt=0.629 min, (747.2 [M+H]⁺).

The following Intermediates in Table 3 were made with non-critical changes or substitutions to the exemplified procedure for Intermediate 1.950 that would be understood by one skilled in the art, wherein R may or may not contain a THP protecting group.

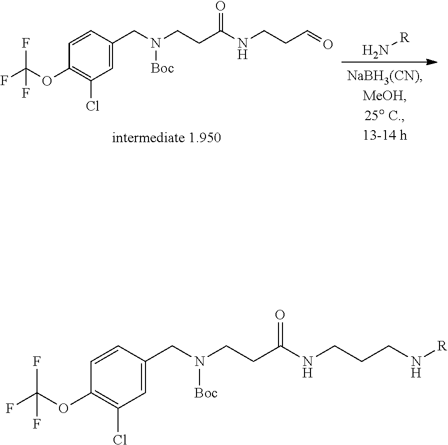

TABLE 3

| Reagent H₂N—R IUPAC | Product Intermediate | |
|---|---|---|
| name/Intermediate | IUPAC name/Intermediate | |
| No. | No. | Analytical |
| 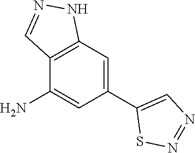 [SMILES: Nc1cc(-c2cnns2)cc2[nH]ncc12] 6-(1,2,3-thiadiazol-5- yl)-1H-indazol-4-amine 1.995 | tert-butyl (3-((3-((6- (1,2,3-thiadiazol-5-yl)- 1H-indazol-4- yl)amino)propyl)amino)- 3-oxopropyl)(3- chloro-4- (trifluoromethoxy) benzyl)carbamate 1.999 | ¹H NMR (400 MHz, MeOH-d₄) δ 9.13 (s, 1H), 8.20 (d, J = 0.8 Hz, 1H), 7.47- 7.40 (m, 1H), 7.36-7.33 (m, 2H), 7.13 (s, 1H), 6.40 (s, 1H), 4.41 (s, 2H), 3.56- 3.51 (m, 2H), 3.38-3.35 (m, 2H), 2.47- 2.45 (m, 2H), 1.97-1.93 (m, 2H), 1.47- 1.36 (m, 9H) ppm. LCMS (AM11): rt = 0.491 min, (654.4 [M + H]⁺), 55% purity. Purification Method PM141 |
| 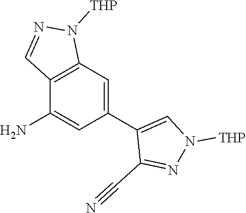 | tert-butyl N-[[3-chloro- 4- (trifluoromethoxy)phenyl] methyl]-N-[3-[3-[[6- (5-cyano-1- tetrahydropyran-2-yl- pyrazol-4-yl)-1- tetrahydropyran-2-yl- indazol-4- yl]amino]propylamino]- 3-oxo- propyl]carbamate 2.226 | ¹H NMR (400 MHz, CHCl₃-d) δ 8.04 (s, 1H), 7.98 (s, 1H), 7.33 (s, 1H), 7.26- 7.24 (m, 1H), 7.16-7.045 (m, 2H), 6.51 (s, 1H), 5.67 (dd, J = 7.2 Hz, 1H), 5.49 (dd, J = 7.2 Hz, 1H), 4.40 (s, 2H), 4.12- 4.01 (m, 2H), 3.80-3.74 (m, 2H), 3.56- 3.46 (m, 2H), 3.44-3.36 (m, 4H), 2.60- 2.42 (m, 3H), 2.22-2.11 (m, 3H), 2.10- 2.03 (m, 2H), 1.97-1.85 (m, 2H), 1.85- 1.66 (m, 6H), 1.43 (s, 9H) ppm. LCMS (AM16): rt = 0.886 min, (829.4 [M + H]⁺), 97.36% purity. Purification Method PM130 |
| 4-(4-amino-1- | | |
| (tetrahydro-2H-pyran-2- | | |
| yl)-1H-indazol-6-yl)-1- | | |
| (tetrahydro-2H-pyran-2- | | |
| yl)-1H-pyrazole-3- | | |
| carbonitrile 1.927 | | |
| 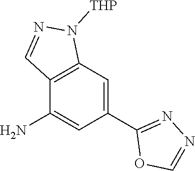 | tert-butyl (3-((3-((6- (1,3,4-oxadiazol-2-yl)- 1-(tetrahydro-2H- pyran-2-yl)-1H- indazol-4- yl)amino)propyl)amino)- 3-oxopropyl)(3- chloro-4- (trifluoromethoxy) benzyl)carbamate 2.233 | LCMS (AM16): rt = 0.804 min, (722.2 [M + H]⁺), 91.00% purity. Purification Method PM131 |
| 6-(1,3,4-oxadiazol-2-yl)- | | |
| 1-(tetrahydro-2H-pyran-2- | | |
| yl)-1H-indazol-4-amine | | |
| 2.231 | | |
| 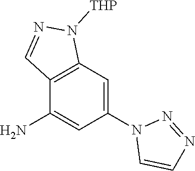 1-(tetrahydro-2H-pyran- 2-yl)-6-(1H-1,2,3-triazol- 1-yl)-1H-indazol-4- amine 2.235 | tert-butyl (3-chloro-4- (trifluoromethoxy) benzyl)(3-oxo-3-((3-((1- (tetrahydro-2H-pyran- 2-yl)-6-(1H-1,2,3- triazol-1-yl)-1H- indazol-4- yl)amino)propyl)amino) propyl)carbamate 2.236 | ¹H NMR (400 MHz, CHCl₃-d) δ 8.14 (s, 1H), 8.06 (d, J = 0.8 Hz, 1H), 7.85 (d, J = 1.2 Hz, 1H), 7.32 (s, 1H), 7.30-7.28 (m, 1H), 7.16 (s, 1H), 7.14-7.09 (m, 1H), 6.62 (s, 1H), 5.68 (dd, J = 9.3 Hz, 2.8 Hz, 1H), 4.40 (s, 2H), 4.07-3.99 (m, 1H), 3.80-3.70 (m, 1H), 3.54 (t, J = 6.0 Hz, 2H), 3.46-3.36 (m, 4H), 2.61- 2.42 (m, 3H), 2.20-2.12 (m, 1H), 2.11- 2.02 (m, 1H), 1.94-1.83 (m, 2H), 1.81- 1.71 (m, 2H), 1.70-1.64 (m, 1H), 1.44 (s, 9H) ppm LCMS (AM16): rt = 0.797 min, (721.3 [M + H]⁺), 96.6% purity. Purification Method PM132 |
| 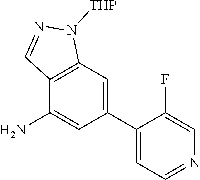 | tert-butyl 3-chloro-4- (trifluoromethoxy)benzyl) (3-((3-((6-(3- fluoropyridin-4-yl)-1- (tetrahydro-2H-pyran- 2-yl)-1H-indazol-4- yl)amino)propyl)amino)- 3-oxopropyl)carbamate 2.239 | LCMS (AM11): rt = 0.523 min, (749.2 [M + H]⁺), 48.87% purity. Purification Method PM133 |
| 6-(3-fluoropyridin-4-yl)- | | |
| 1-(tetrahydro-2H-pyran- | | |
| 2-yl)-1H-indazol-4- | | |
| amine 2.238 | | |
| 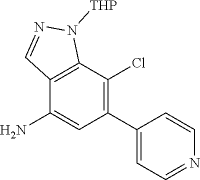 7-chloro-6-(pyridin-4-yl)- 1-(tetrahydro-2H-pyran- 2-yl)-1H-indazol-4- amine 2.024 | tert-butyl (3-chloro-4- (trifluoromethoxy)benzyl) (3-((3-((7-chloro-6- (pyridin-4-yl)-1- (tetrahydro-2H-pyran- 2-yl)-1H-indazol-4- yl)amino)propyl)amino)- 3-oxopropyl)carbamate 2.249 | ¹H NMR (400 MHz, MeOH-d₄) δ 8.58 (dd, J = 4.4 Hz, 1.6 Hz, 2H), 8.26 (s, 1H), 7.50 (dd, J = 4.4 Hz, 2.0 Hz, 2H), 7.39-7.33 (m, 2H), 7.19 (dd, J = 8.0 Hz, 2.0 Hz, 1H), 6.47-6.41 (m, 1H), 6.15 (s, 1H), 4.38 (s, 2H), 4.01-3.94 (m, 1H), 3.82-3.71 (m, 1H), 3.52-3.48 (m, 2H), 3.30-3.23 (m, 2H), 2.45-2.38 (m, 2H), 2.15-2.05 (m, 2H), 1.93-1.85 (m, 2H), 1.83-1.56 (m, 4H), 1.51-1.32 (m, 11H) ppm. LCMS (AM16): rt = 0.868 min, (765.2 [M + H]⁺), 96.48% purity. Purification Method PM158 |
| 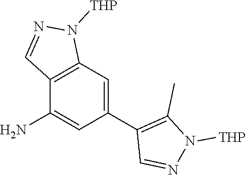 6-(5-methyl-1-(tetrahydro-2H-pyran-2- yl)-1H-pyrazol-4-yl)-1-(tetrahydro- 2H-pyran-2- yl)-1H-indazol-4-amine 2.251 | tert-butyl (3-chloro-4- (trifluoromethoxy)benzyl) (3-((3-((6-(5-methyl- 1-(tetrahydro-2H- pyran-2-yl)-1H- pyrazol-4-yl)-1- (tetrahydro-2H-pyran- 2-yl)-1H-indazol-4- yl)amino)propyl)amino)- 3-oxopropyl)carbamate 2.252 | ¹H NMR (400 MHz, MeOH-d₄) δ 8.13 (s, 1H), 7.93 (s, 1H), 7.37-7.29 (m, 2H), 7.18-7.12 (m, 1H), 8.86 (s, 1H), 8.27 (s, 1H), 5.70 (dd, J = 8.0 Hz, 2.4 Hz, 1H), 5.36-5.26 (m, 1H), 4.38 (s, 2H), 4.09-3.97 (m, 2H), 3.84-3.71 (m, 2H), 3.53-3.47 (m, 2H), 2.58-2.42 (m, 4H), 2.42-2.36 (m, 3H), 2.21-1.93 (m, 4H), 1.80-1.59 (m, 6H), 1.50-1.27 (m, 11H) ppm. LCMS (AM16): rt = 0.849 min, (818.4 [M + H]⁺), 93.9% purity. Purification Method PM159 |
| 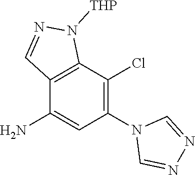 | tert-butyl (3-((3-((7- chloro-1-(tetrahydro- 2H-pyran-2-yl)-6- (4H-1,2,4-triazol-4- yl)-1H-indazol-4- yl)amino)propyl)amino)- 3-oxopropyl)(3-chloro-4- (trifluoromethoxy) benzyl)carbamate 2.322 | LCMS (AM14): rt = 0.694 min, (755.3 [M + H]⁺), 100% purity. Purification Method PM172 |
| 7-chloro-1-(tetrahydro- | | |
| 2H-pyran-2-yl)-6-(4H- | | |
| 1,2,4-triazol-4-yl)-1H- | | |
| indazol-4-amine 2.321 | | |
| 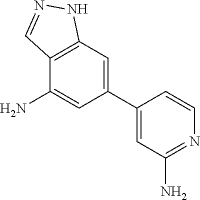 [SMILES: Nc1cc(-c2cc(N)c3cn[nH]c3c2)ccn1] | tert-butyl (3-((3-((6-(2- aminopyridin-4-yl)-1H- indazol-4- yl)amino)propyl)amino)- 3-oxopropyl)(3- chloro-4- (trifluoromethoxy) benzyl)carbamate 2.323 | LCMS (AM11): rt = 0.447 min, (662.5 [M + H]⁺), 98.5% purity. Purification Method PM173 |
| 6-(2-aminopyridin-4-yl)- | | |
| 1H-indazol-4-amine | | |
| 2.306 | | |
| 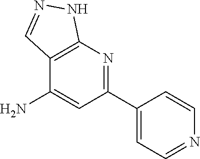 [SMILES: Nc1cc(-c2ccncc2)nc2[nH]ncc12] | tert-butyl (3-chloro-4- (trifluoromethoxy)benzyl) (3-oxo-3-((3-((6- (pyridin-4-yl)-1H- pyrazolo[3,4-b]pyridin- 4-yl)amino)propyl) amino)propyl)carbamate 2.324 | LCMS (AM12): rt = 0.517 min, (648.1 [M + H]⁺), 80.48% purity. Purification Method PM174 |
| 6-(pyridin-4-yl)-1H- | | |
| pyrazolo[3,4-b]pyridin- | | |
| 4-amine 2.313 | | |
| 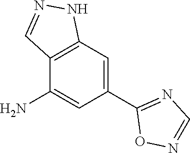 [SMILES: Nc1cc(-c2ncno2)cc2[nH]ncc12] | tert-butyl (3-((3-((6- (1,2,4-oxadiazol-5-yl)- 1H-indazol-4- yl)amino)propyl)amino)- 3-oxopropyl)(3- chloro-4- (trifluoromethoxy) benzyl)carbamate 2.336 | LCMS (AM12): rt = 0.619 min, (638.4 [M + H]⁺), 82.50% purity. Purification Method PM176 |
| 6-(1,2,4-oxadiazol-5-yl)- | | |
| 1H-indazol-4-amine | | |
| 2.335 | | |
| 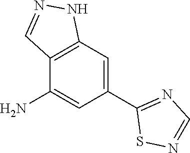 [SMILES: Nc1cc(-c2ncns2)cc2[nH]ncc12] | tert-butyl (3-((3-((6- (1,2,4-thiadiazol-5-yl)- 1H-indazol-4- yl)amino)propyl)amino)- 3-oxopropyl)(3- chloro-4- (trifluoromethoxy) benzyl)carbamate 2.374 | LCMS (AM11): rt = 0.525 min, (676.5 [M + Na]⁺), 75.49% purity. Purification Method PM144 |
| 6-(1,2,4-thiadiazol-5-yl)- | | |
| 1H-indazol-4-amine | | |
| 2.373 | | |
| 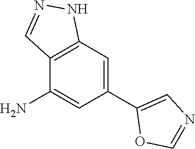 [SMILES: Nc1cc(-c2cnco2)cc2[nH]ncc12] | tert-butyl 3-chloro-4- (trifluoromethoxy)benzyl (3-((3-((6-(oxazol-5- yl)-1H-indazol-4- yl)amino)propyl)amino)- 3-oxopropyl)carbamate 2.389 | LCMS (AM11): rt = 0.596 min, (637.5 [M + H]⁺), 89.3% purity Purification Method PM192 |
| 6-(oxazol-5-yl)-1H- | | |
| indazol-4-amine 2.388 | | |
| 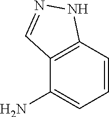 | tert-butyl (3-((3-((1H- indazol-4- yl)amino)propyl)amino)- 3-oxopropyl)(3- chloro-4- (trifluoromethoxy) benzyl)carbamate 2.390 | LCMS (AM16): rt = 0.802 min, (570.2, [M + H]⁺), 93.9% purity. Purification Method PM1131 |
| 1H-indazol-4-amine | | |
| 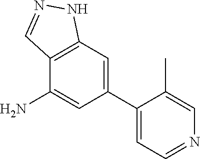 | tert-butyl (3-chloro-4- (trifluoromethoxy) benzyl)(3-((3-((6-(3- methylpyridin-4-yl)-1H- indazol-4- yl)amino)propyl)amino)- 3-oxopropyl)carbamate 2.393 | LCMS (AM11): rt = 0.417 min, (661.5, [M + H]⁺), 92.0% purity. Purification Method PM1194 |
| 6-(3-methylpyridin-4-yl)- | | |
| 1H-indazol-4-amine | | |
| 2.392 | | |

### Synthesis of Intermediate 1.953

### 1-(tetrahydro-2H-pyran-2-yl)-6-(4,4,5,5-tetramethyl-1,3,2-dioxaborolan-2-yl)-1H-indazol-4-amine 1.952

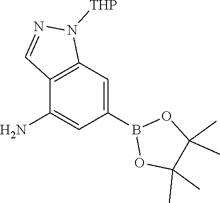

To a solution of compound 1.381 (1.71 g, 5.77 mmol) in dioxane (30 mL) was added 4,4,4′,4′,5,5,5′,5′-octamethyl-2,2′-bi(1,3,2-dioxaborolane) (1.76 g, 6.93 mmol) followed by KOAc (1.13 g, 11.55 mmol) and Pd(dppf)Cl₂ (422.48 mg, 577.39 μmol). The mixture was degassed and purged with N₂ (×3), then the mixture heated to 90° C. and stirred for 3 h. The reaction mixture was cooled to 20° C., diluted with EA (90 mL) and filtered. The filter cake was washed with EA (40 mL×2) and the filtrate was concentrated in vacuo to give a residue, which was purified (PM39) to afford compound 1.952 (1.96 g, 3.78 mmol, 65.46% yield) as a brown oil.

LCMS (AM11): rt=0.444 min, (344.1 [M+H]⁺), 66.19% purity.

¹H NMR (400 MHz, CHCl₃-d) δ 8.00 (s, 1H), 7.43 (s, 1H), 6.80 (s, 1H), 5.73 (dd, J=10.0 Hz, 2.4 Hz, 1H), 4.10-4.05 (m, 1H), 3.81-3.70 (m, 1H), 3.67 (br s, 2H), 2.61-2.57 (m, 1H), 2.16-2.10 (m, 1H), 2.05-1.92 (m, 1H), 1.81-1.71 (m, 2H), 1.67-1.58 (m, 1H), 1.37 (s, 12H) ppm.

### 5-(4-amino-1-(tetrahydro-2H-pyran-2-yl)-1H-indazol-6-yl)pyridazin-3(2H)-one 1.953

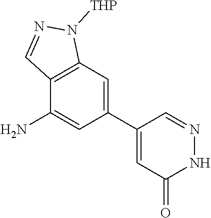

To a solution of compound 1.952 (500 mg, 1.46 mmol) in dioxane (5 mL) and H₂O (1 mL) was added 5-chloropyridazin-3(2H)-one (190.15 mg, 1.46 mmol), K₂CO₃ (604.00 mg, 4.37 mmol) and Pd(dppf)Cl₂ (106.59 mg, 145.68 μmol). The mixture was stirred at 100° C. for 16 h under N₂. The reaction mixture was poured onto H₂O (10 mL) and extracted with EA (10 mL×3). The combined organic phase was washed (brine, 10 mL), dried (Na₂SO₄), filtered and concentrated in vacuo to give a residue, which was triturated with EA (5 mL) at 20° C. for 10 minutes, filtered and the filter cake was dried under vacuum, to afford compound 1.953 (176 mg, 463.55 μmol, 31.82% yield) as a brown solid.

LCMS (AM16): rt=0.436 min, (312.2 [M+H]⁺), 82.51% purity.

### Synthesis of Intermediate 1.995

### 6-allyl-4-nitro-1-(tetrahydro-2H-pyran-2-yl)-1H-indazole 1.991

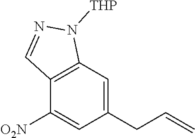

To a mixture of 6-bromo-4-nitro-1-(tetrahydro-2H-pyran-2-yl)-1H-indazole (WO2019027960, 1 g, 3.07 mmol) and 2-allyl-4,4,5,5-tetramethyl-1,3,2-dioxaborolane (772.85 mg, 4.60 mmol) in dioxane (12 mL) and H₂O (3 mL) under N₂ was added K₂CO₃ (1.27 g, 9.20 mmol) followed by Pd(dppf)Cl₂ (224.35 mg, 306.61 μmol). The mixture was heated to 80° C. and stirred for 2 h. The mixture was filtered and the filtrate was concentrated in vacuo to give a residue, which was purified (PM139) to afford compound 1.991 (800 mg, 2.45 mmol, 79.92% yield) as a brown oil.

LCMS (AM14): rt=0.684 min, (288.2 [M+H]⁺), 88.08% purity.

¹H NMR (400 MHz, DMSO-d₆) δ 8.51 (s, 1H), 8.14 (s, 1H), 8.06 (d, J=0.8 Hz, 1H), 6.13-6.03 (m, 1H), 6.00 (dd, J=9.6, 2.4 Hz, 1H), 5.24-5.15 (m, 2H), 3.88 (br s, 1H), 3.83-3.74 (m, 1H), 3.68 (d, J=6.8 Hz, 2H), 2.46-2.37 (m, 1H), 1.81-1.71 (m, 1H), 1.64-1.59 (m, 2H) ppm.

### 2-(4-nitro-1-(tetrahydro-2H-pyran-2-yl)-1H-indazol-6-yl)acetaldehyde 1.992

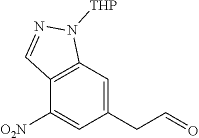

To a solution of compound 1.991 (466 mg, 1.14 mmol, 70% purity) in THF (20 mL) and H₂O (10 mL) at 0° C. was added K₂OsO₄·2H₂O (83.67 mg, 227.07 μmol) and NaIO₄ (728.52 mg, 3.41 mmol) portionwise and the mixture stirred at 0° C. for 1 h. The mixture was poured into water (100 mL) and extracted with EA (80 mL×3). The combined organic phase was washed with Na₂SO₃ aqueous solution (40 mL×2) and brine (40 mL×4), dried (Na₂SO₄), filtered and concentrated in vacuo to give a residue, which was purified (PM38) to afford compound 1.992 (644 mg, 2.23 mmol, 32.68% yield) as a yellow solid.

¹H NMR (400 MHz, CHCl₃-d) δ 9.93-9.86 (m, 1H), 8.63 (s, 1H), 8.04 (s, 1H), 7.85 (s, 1H), 5.81 (dd, J=8.8, 2.8 Hz, 1H), 4.01 (d, J=1.2 Hz, 2H), 4.00-3.93 (m, 1H), 3.82-3.74 (m, 1H), 2.60-2.49 (m, 1H), 2.24-2.13 (m, 2H), 1.81-1.70 (m, 3H) ppm.

### methyl 2-(2-(4-nitro-1-(tetrahydro-2H-pyran-2-yl)-1H-indazol-6-yl)ethylidene)hydrazine-1-carboxylate 1.993

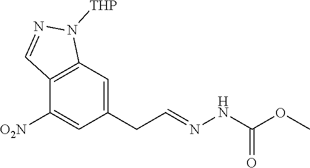

To a solution of compound 1.992 (644 mg, 2.23 mmol) and ethyl N-aminocarbamate (254.94 mg, 2.45 mmol) in EtOH (15 mL) was added HOAc (133.68 mg, 2.23 mmol) at 20° C., then the mixture was heated to 50° C. and stirred for 0.5 h. The mixture was concentrated in vacuo to afford compound 1.993 (800 mg, 2.13 mmol, 95.73% yield) as a yellow solid.

LCMS (AM11): rt=0.413 min, (291.8 [M+H]⁺), 81.95% purity.

### 5-(4-nitro-1H-indazol-6-yl)-1,2,3-thiadiazole 1.994

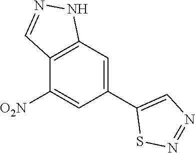 [SMILES: O=[N+]([O-])c1cc(-c2cnns2)cc2[nH]ncc12]

A solution of compound 1.993 (800 mg, 2.13 mmol) in SOCl₂ (10 mL) was stirred at 80° C. for 0.5 h. The reaction mixture was concentrated in vacuo to give a residue. Saturated NaHCO₃ aq. solution (40 mL) was added and the mixture extracted with EA (40 mL×2). The combined organic phase was washed (brine, 20 mL×2), dried (Na₂SO₄), filtered and concentrated in vacuo to give a residue, which was purified (PM140) to afford compound 1.994 (200 mg, 760.17 μmol, 35.67% yield) as a yellow solid.

LCMS (AM12): rt=0.419 min, (247.9 [M+H]⁺), 93.97% purity.

¹H NMR (400 MHz, DMSO-d₆) δ 14.23 (br s, 1H), 9.68 (s, 1H), 8.62 (s, 1H), 8.55-8.46 (m, 2H) ppm.

### 6-(1,2,3-thiadiazol-5-yl)-1H-indazol-4-amine 1.995

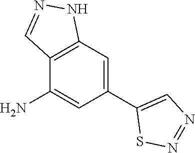 [SMILES: Nc1cc(-c2cnns2)cc2[nH]ncc12]

To a mixture of compound 1.994 (70 mg, 283.13 μmol) in EA (2 mL) was added SnCl₂·2H₂O (191.66 mg, 849.40 μmol). The mixture was heated to 70° C. and stirred for 0.5 h. The reaction mixture was poured into water (20 mL) and extracted with EA (20 mL×3). The combined organic phase was washed (brine, 20 mL×2), dried (Na₂SO₄), filtered and concentrated in vacuo to give a residue, which was triturated with EA (2 mL) at 20° C. for 5 min. The filter cake was dried in vacuo to afford compound 1.995 (60 mg, 207.13 μmol, 73.16% yield) as a yellow solid.

LCMS (AM11): rt=0.275 min, (218.0 [M+H]⁺), 75.30% purity.

¹H NMR (400 MHz, DMSO-d₆) δ 12.98 (s, 1H), 9.32 (s, 1H), 8.17 (s, 1H), 7.10 (s, 1H), 6.44 (s, 1H), 6.12 (s, 2H) ppm.

### Synthesis of Intermediate 2.001

### tert-butyl (4-(2-((6-(2-methoxypyridin-4-yl)-1-(tetrahydro-2H-pyran-2-yl)-1H-indazol-4-yl)amino)ethoxy)butyl)carbamate 2.000

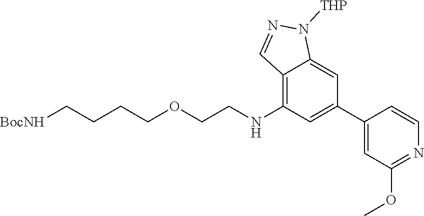

To a solution of compound 1.893 (0.68 g, 1.10 mmol) in dioxane (5 mL) and H₂O (0.5 mL) was added K₂CO₃ (305.40 mg, 2.21 mmol), 2-methoxypyridin-4-yl)boronic acid (311.68 mg, 1.33 mmol) and Brettphos Pd G3 (100.16 mg, 0.11 mmol), the mixture was stirred at 80° C. for 12 h. The mixture was concentrated in vacuo to give a residue, which was purified (PM239) to afford compound 2.000 (550 mg, 84.5% yield) as a light-yellow solid.

LCMS (AM3): rt=0.846 min, (540.2 [M+H]⁺), 91.6% purity.

### N-(2-(4-aminobutoxy)ethyl)-6-(2-methoxypyridin-4-yl)-1H-indazol-4-amine 2.001

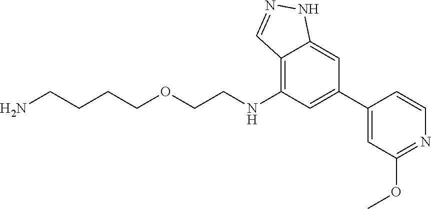 [SMILES: COc1cc(-c2cc(NCCOCCCCN)c3cn[nH]c3c2)ccn1]

To a solution of compound 2.000 (500 mg, 0.92 mmol) in DCM (10 mL) was added TFA (15.40 g, 135.06 mmol), the mixture was stirred at RT for 0.5 h. The mixture was concentrated in vacuo to give a residue, which was purified (PM106). H₂O (30 mL) was added and the mixture adjusted to pH=7-8 (sat. NaHCO₃ (aq.)) and extracted with EA (50 mL×2). The combined organic phase was dried (Na₂SO₄), filtered and concentrated in vacuo to afford compound 2.001 (230 mg, 64.6% yield) as light-yellow oil.

LCMS (AM3): rt=0.603 min, (356.2 [M+H]⁺), 92.5% purity

### Synthesis of Intermediate 2.006

### 6-(4-methyl-1-((2-(trimethylsilyl)ethoxy)methyl)-1H-imidazol-5-yl)-4-nitro-1-(tetrahydro-2H-pyran-2-yl)-1H-indazole 2.003

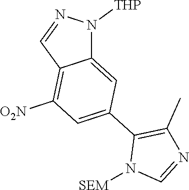

To a solution of compound 1.943 (0.5 g, 1.34 mmol) in dioxane (10 mL) and H₂O (1 mL) was added K₂CO₃ (370.32 mg, 2.68 mmol), 4-bromo-5-methyl-1-[[2-(trimethylsilyl)ethoxy]methyl]-1H-imidazole (CAS 1222175-06-5, 468.25 mg, 1.61 mmol) and Brettphos G3 (121.45 mg, 0.13 mmol). The mixture was stirred at 80° C. for 12 h under N₂. The mixture was concentrated in vacuo to give a residue, which was purified (PM142) to afford compound 2.003 (280 mg, 43.2% yield) as light-yellow oil.

LCMS (AM3): rt=0.835 min, (458.3 [M+H]⁺), 94.7% purity.

### 6-(4-methyl-1-((2-(trimethylsilyl)ethoxy)methyl)-1H-imidazol-5-yl)-1-(tetrahydro-2H-pyran-2-yl)-1H-indazol-4-amine 2.004

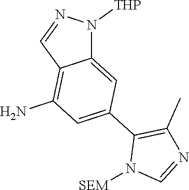

To a solution of compound 2.003 (260 mg, 0.57 mmol) in THF (10 mL) was added Pd/C (260 mg, 0.056 mmol, 10% purity) under N₂. The suspension was degassed and purged with H₂ (×3). The mixture was stirred at 25° C. for 12 h under H₂ (15 Psi). The mixture was filtered and the filtrate was concentrated in vacuo to give a residue, which was purified (PM144) to afford compound 2.004 (210 mg, 73.6% yield) as a light-yellow oil.

LCMS (AM3): rt=0.854 min, (428.3 [M+H]⁺), 85.2% purity.

### tert-butyl ((2-chloro-[1,1′-biphenyl]-4-yl)methyl)(3-((3-((6-(4-methyl-1-((2-(trimethylsilyl)ethoxy)methyl)-1H-imidazol-5-yl)-1-(tetrahydro-2H-pyran-2-yl)-1H-indazol-4-yl)amino)propyl)amino)-3-oxopropyl)carbamate 2.005

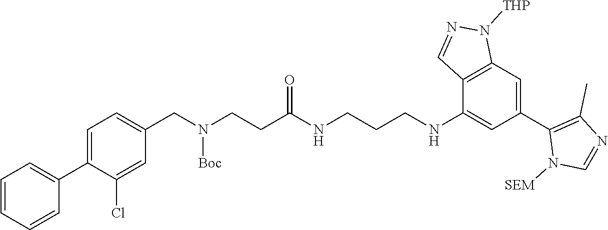

To a solution of compound 2.004 (200 mg, 0.39 mmol) in MeOH (10 mL) was added Intermediate H (265.96 mg, 0.59 mmol). After 11 h, NaBH₃CN (250.42 mg, 3.98 mmol) was added. The mixture was stirred at 25° C. for 1 h. The resultant mixture was then concentrated in vacuo to give a residue, which was purified (PM145) to afford compound 2.005 (160 mg, 41.3% yield) as a light-yellow oil.

LCMS (AM3): rt=0.921 min, (856.2 [M+H]⁺), 88.2% purity.

### tert-butyl ((2-chloro-[1,1′-biphenyl]-4-yl)methyl)(3-((3-((6-(4-methyl-1H-imidazol-5-yl)-1-(tetrahydro-2H-pyran-2-yl)-1H-indazol-4-yl)amino)propyl)amino)-3-oxopropyl)carbamate 2.006

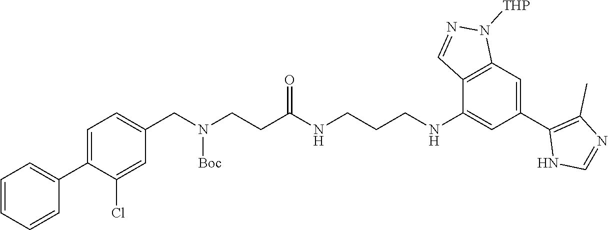

Compound 2.005 (150 mg, 0.15 mmol) was dissolved in THF (5 mL) and TBAF (1 M, 1.54 mL) was added. The mixture was stirred at 70° C. for 1 h. The resultant mixture was concentrated in vacuo to give a residue, which was purified (PM146) to afford compound 2.006 (50 mg, 38.2% yield) as a light-yellow oil.

LCMS (AM3): rt=0.842 min, (726.2 [M+H]⁺), 85.8% purity.

### Synthesis of Intermediate 2.008

### 6-(furan-3-yl)-4-nitro-1-(tetrahydro-2H-pyran-2-yl)-1H-indazole 2.007

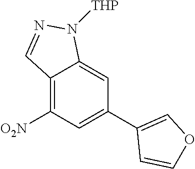

To a solution of 6-bromo-4-nitro-1-(tetrahydro-2H-pyran-2-yl)-1H-indazole (WO2019027960, 400 mg, 1.23 mmol) in dioxane (10 mL) and H₂O (1 mL) was added K₃PO₄ (520.67 mg, 2.45 mmol), furan-3-ylboronic acid (164.67 mg, 1.47 mmol) and Pd(dppf)Cl₂ (89.74 mg, 0.122 mmol). The mixture was stirred at 80° C. for 12 h under N₂. The mixture was concentrated in vacuo to give a residue, which was purified (PM46) to afford compound 2.007 (320 mg, 83.2% yield) as a yellow solid.

¹H NMR (400 MHz, MeOH-d₄) δ: 8.54 (s, 1H), 8.39 (s, 1H), 8.29 (s, 1H), 8.21 (s, 1H), 7.68 (s, 1H), 7.05 (s, 1H), 6.02 (dd, J=9.2, 2.8 Hz, 1H), 4.02-3.99 (m, 2H), 3.93-3.89 (m, 2H), 2.58-2.55 (m, 2H), 2.17-2.16 (m, 1H), 2.13-2.12 (m, 1H), 1.88-1.86 (m, 1H), 1.75-1.71 (m, 2H) ppm

### 1-(tetrahydro-2H-pyran-2-yl)-6-(tetrahydrofuran-3-yl)-1H-indazol-4-amine 2.008

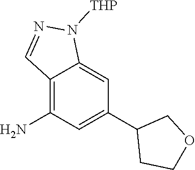

To a solution of compound 2.007 (320 mg, 1.02 mmol) in MeOH (10 mL) was added Pd/C (0.3 g, 0.102 mmol, 10% purity) under N₂. The suspension was degassed and purged with H₂ (×3). The mixture was stirred at 25° C. for 12 h under H₂ (15 psi). The mixture was neutralized to pH=7 with formic acid. The mixture was concentrated in vacuo to give a residue, which was purified (PM39) to afford compound 2.008 (260 mg, 84.7% yield) as a red oil.

LCMS (AM3): rt=0.768 min, (288.1 [M+H]⁺), 95.7% purity.

### Synthesis of Intermediate 2.010

### 6-(oxetan-3-yl)-1-(tetrahydro-2H-pyran-2-yl)-1H-indazol-4-amine 2.010

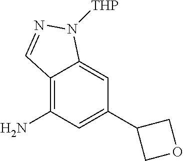

To a 8 mL vial equipped with a stir bar was added compound 1.381 (500 mg, 1.69 mmol), 3-bromooxetane (300.62 mg, 2.19 mmol), NiCl₂·dtbbpy (3.36 mg, 0.008 mmol), bis[3,5-difluoro-2-[5-(trifluoromethyl)-2-pyridyl]phenyl]iridium(1+); 4-tertbutyl-2-(4-tert-butyl-2-pyridyl) pyridine; hexafluorophosphate (18.94 mg, 0.017 mmol), bis(trimethylsilyl)silyl-trimethyl-silane (419.80 mg, 1.69 mmol) and Na₂CO₃ (357.87 mg, 3.38 mmol) and DME (10 mL). The vial was purged with N₂ and sealed. The reaction was stirred and irradiated with a 34 W blue LED lamp (7 cm away) with cooling fan to keep the reaction temperature at 25° C. for 12 h. The mixture was concentrated in vacuo to give a residue, which was purified (PM102) to afford compound 2.010 (180 mg, 18.7% yield) as a black-brown oil.

LCMS (AM3): rt=0.768 min, (274.1 [M+H]⁺), 48.0% purity.

### Synthesis of Intermediate 2.014

### 4-amino-1-(tetrahydro-2H-pyran-2-yl)-1H-indazole-6-carbonitrile 2.012

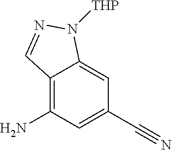

To a solution of compound 1.381 (1 g, 3.38 mmol) in DMF (10 mL) was added Pd(PPh₃)₄ (390.18 mg, 0.34 mmol) and Zn(CN)₂ (1.19 g, 10.13 mmol). The mixture was stirred at 100° C. for 12 h under N₂. The mixture was filtered and the filtrate was concentrated in vacuo to give a residue, which was purified (PM44) to afford compound 2.012 (730 mg, 74.7% yield) as a light-yellow oil.

LCMS (AM3): rt=0.637 min, (242.9 [M+H]⁺), 83.8% purity.

### tert-butyl ((2-chloro-[1,1′-biphenyl]-4-yl)methyl)(3-((3-((6-cyano-1-(tetrahydro-2H-pyran-2-yl)-1H-indazol-4-yl)amino)propyl)amino)-3-oxopropyl)carbamate 2.013

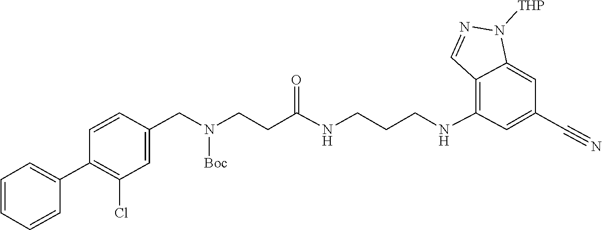

To a solution of compound 2.012 (400 mg, 1.38 mmol) in MeOH (1 mL) was added Intermediate H (1.09 g, 2.08 mmol). 1 h later, NaBH₃CN (869.45 mg, 13.84 mmol) was added. The mixture was stirred at 25° C. for 11 h. The mixture was filtered and the filtrate was concentrated in vacuo to give a residue, which was purified (PM90) to afford compound 2.013 (520 mg, 54.9% yield) as black-brown oil.

LCMS (AM3): rt=1.137 min, (671.4 [M+H]⁺), 98.2% purity.

### tert-butyl ((2-chloro-[1,1′-biphenyl]-4-yl)methyl)(3-oxo-3-((3-((1-(tetrahydro-2H-pyran-2-yl)-6-(2H-tetrazol-5-yl)-1H-indazol-4-yl)amino)propyl)amino)propyl)carbamate 2.014

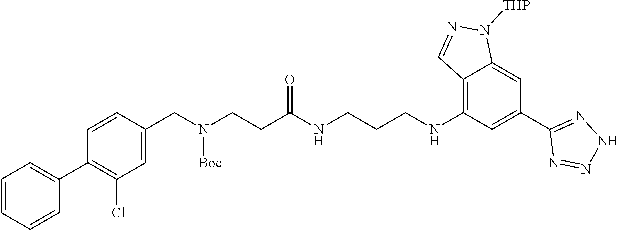

To a solution of compound 2.013 (470 mg, 0.70 mmol) in DMF (4 mL) was added NaN₃ (910.41 mg, 14.0 mmol) and NH₄Cl (749.10 mg, 14.0 mmol). The mixture was stirred at 100° C. for 12 h. The mixture was filtered and the filtrate was concentrated in vacuo to give a residue, which was purified (PM104) to afford compound 2.014 (200 mg, 0.27 mmol, 38.4% yield) as a yellow solid.

LCMS (AM3): rt=1.042 min, (714.3 [M+H]⁺), 96.2% purity.

### Synthesis of Intermediate 2.016

### 5-(4-nitro-1-(tetrahydro-2H-pyran-2-yl)-1H-indazol-6-yl)pyridazin-3-amine 2.015

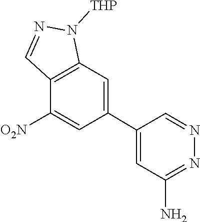

To a solution of compound 1.943 (100 mg, 267.95 μmol) in dioxane (1 mL) and H₂O (0.1 mL) was added 5-chloropyridazin-3-amine (34.71 mg, 267.95 μmol), K₂CO₃ (111.10 mg, 803.84 μmol) and Pd(dppf)Cl₂ (19.61 mg, 26.79 μmol) at 20° C. The reaction mixture was degassed and purged with nitrogen for 3 times and stirred at 80° C. for 12 h. The mixture was concentrated in vacuo to give a residue, which was purified (PM23) to afford compound 2.015 (90 mg, 264.44 μmol, 98.69% yield) as a yellow solid.

LCMS (AM3): rt=0.780 min, (340.8 [M+H]⁺), 57.26% purity.

### 6-(6-aminopyridazin-4-yl)-1-(tetrahydro-2H-pyran-2-yl)-1H-indazol-4-amine 2.016

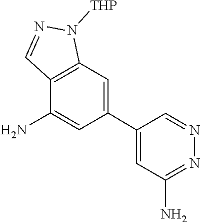

To a solution of compound 2.015 (90 mg, 264.44 μmol) in EtOH (1 mL) and H₂O (0.2 mL) was added Fe (73.84 mg, 1.32 mmol) and NH₄Cl (70.73 mg, 1.32 mmol) at 25° C. The mixture was stirred at 80° C. for 2 h. The mixture was filtered and the filter cake was washed with EtOH (10 mL×3). The combined filtrate was concentrated in vacuo to afford compound 2.016 (100 mg, crude) as a yellow solid.

LCMS (AM3): rt=0.385 min, (311.2 [M+H]⁺), 92.27% purity

### Synthesis of Intermediate 2.021

### 4-amino-1-(tetrahydro-2H-pyran-2-yl)-1H-indazole-6-carboximidhydrazide 2.018

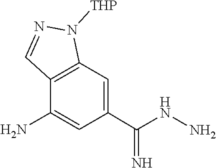

To a mixture of compound 2.012 (800 mg, 3.30 mmol) in NaOMe (2.45 M, 10 mL) was added NH₂NH₂·H₂O (3.52 g, 70.27 mmol, 3.42 mL). The mixture was stirred at 80° C. for 12 hours. The mixture was concentrated in vacuo to give a residue, which was purified (PM105) to afford compound 2.018 (700 mg, 2.14 mmol, 64.90% yield, 98.068% purity, FA salt) as an off-white solid.

LCMS (AM5): rt=0.631 min, (275.2 [M+H]⁺), 98.068% purity.

¹H NMR (400 MHz, DMSO-d₆) δ 8.39 (s, 2H), 8.13 (s, 1H), 7.09 (s, 1H), 6.58 (s, 1H), 5.91 (br. s, 2H), 5.67-5.64 (m, 1H), 3.88-3.80 (m, 1H), 3.71-3.68 (m, 1H), 2.03-2.00 (m, 1H), 1.90-1.87 (m, 1H), 1.72-1.70 (m, 1H), 1.57-1.56 (m, 3H) ppm.

### 1-(tetrahydro-2H-pyran-2-yl)-6-(4H-1,2,4-triazol-3-yl)-1H-indazol-4-amine 2.019

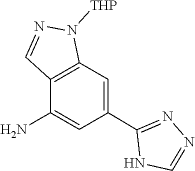

A solution of compound 2.018 (700 mg, 2.19 mmol, FA salt) and trimethoxymethane (1.16 g, 10.93 mmol, 1.20 mL) in EtOH (10 mL) was stirred at 80° C. for 5 h. The mixture was concentrated in vacuo to give a residue, which was purified (PM106) to afford compound 2.019 (200 mg, 703.44 μmol, 32.19% yield) as a yellow solid.

¹H NMR (400 MHz, DMSO-d₆) δ 14.23 (s, 1H), 8.21 (s, 1H), 7.47 (s, 1H), 7.00 (s, 1H), 6.06 (s, 1H), 5.77 (dd, J=10.0, 2.0 Hz, 1H), 3.98-3.95 (m, 1H), 3.82-3.77 (m, 1H), 2.48-2.45 (m, 1H), 2.11-2.07 (m, 1H), 2.02-1.96 (m, 1H), 1.84-1.82 (m, 1H), 1.65-1.64 (m, 2H) ppm.

### 6-(4H-1,2,4-triazol-3-yl)-1H-indazol-4-amine 2.020

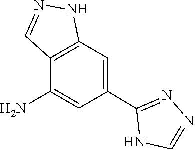 [SMILES: Nc1cc(-c2nnc[nH]2)cc2[nH]ncc12]

To a solution of compound 2.019 (20 mg, 70.34 μmol) in DCM (0.5 mL) was added TFA (770.00 mg, 6.75 mmol, 0.5 mL) dropwise. The reaction was stirred at 25° C. for 1 h. The mixture was concentrated in vacuo to give a residue, which was purified (PM107) to afford compound 2.020 (3.55 mg, 17.73 μmol, 25.21% yield) as an off-white solid.

LCMS (AM5): rt=0.372 min, (201.1 [M+H]⁺), 100.00% purity.

¹H NMR (400 MHz, CHCl₃-d): δ 8.30 (s, 1H), 8.13 (s, 1H), 7.44 (s, 1H), 6.92 (s, 1H) ppm.

### tert-butyl (3-chloro-4-(trifluoromethoxy)benzyl)(3-oxo-3-((3-((1-(tetrahydro-2H-pyran-2-yl)-6-(4H-1,2,4-triazol-3-yl)-1H-indazol-4-yl)amino)propyl)amino)propyl)carbamate 2.021

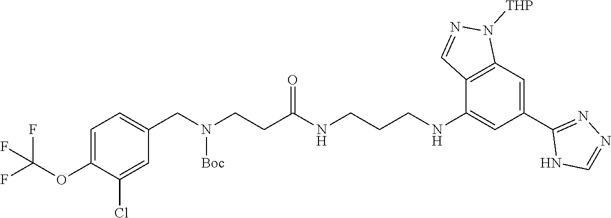

To a solution of compound 2.020 (70 mg, 164.94 μmol, 66.994% purity) and compound 1.325 (74.69 mg, 164.94 μmol) in MeOH (1 mL) was added MgSO₄ (99.27 mg, 824.70 mol) and AcOH (9.90 mg, 164.94 μmol, 9.43 L). The reaction was stirred at 20° C. for 12 h. NaBH₃CN (36.28 mg, 577.29 μmol) was added and the mixture was stirred at 20° C. for 2 h. The mixture was filtered and the filter cake was washed with methanol (10 mL×3). The combined filtrate was concentrated in vacuo to give a residue, which was purified (PM91) to afford compound 2.021 (70 mg, 82.26 μmol, 49.87% yield) as an off-white solid.

LCMS (AM7): rt=0.909 min, (721.2 [M+H]⁺), 84.751% purity.

### Synthesis of Intermediate 2.026

### 6-bromo-7-chloro-1-(tetrahydro-2H-pyran-2-yl)-1H-indazol-4-amine 2.023

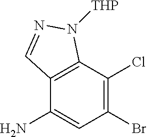

To a solution of compound 1.381 (3.08 g, 10.40 mmol) in DMF (30 mL) was added NCS (1.53 g, 11.44 mmol) at 0° C. The resulting mixture was stirred at 0° C. for 1 h. Water (100 mL) was added and the mixture was extracted with EA (150 mL×3). The combined organic phase was washed (brine, 100 mL×2), dried (Na₂SO₄), filtered and concentrated in vacuo to give a residue, which was purified (PM39) to afford compound 2.023 (1 g, 2.90 mmol, 27.89% yield) as a brown oil.

LCMS (AM3): rt=0.915 min, (354.0 [M+Na]⁺), 95.90% purity.

¹H NMR (400 MHz, CHCl₃-d) δ 7.93 (s, 1H), 6.66 (s, 1H), 6.39 (dd, J=9.6, 2.4 Hz, 1H), 4.04-4.02 (m, 1H), 3.82-3.74 (m, 1H), 2.66-2.57 (m, 1H), 2.17-2.10 (m, 2H), 1.79-1.60 (m, 3H) ppm.

### 7-chloro-6-(pyridin-4-yl)-1-(tetrahydro-2H-pyran-2-yl)-1H-indazol-4-amine 2.024

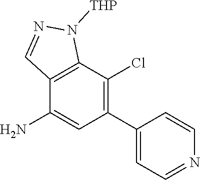

To a solution of compound 2.023 (1 g, 3.02 mmol) and pyridin-4-yl boronic acid (371.79 mg, 3.02 mmol) in dioxane (10 mL) and H₂O (1 mL) was added K₂CO₃ (836.07 mg, 6.05 mmol) and Pd(dppf)Cl₂ (221.32 mg, 302.47 μmol) at 20° C. under N₂. The mixture was heated to 90° C. and stirred for 16 h. Water (30 mL) was added and the mixture was extracted with EA (50 mL×3). The combined organic phase was dried (Na₂SO₄), filtered and concentrated in vacuo to give a residue, which was purified (PM30) to afford compound 2.024 (300 mg, 886.08 μmol, 29.29% yield) as a yellow solid.

LCMS (AM3): rt=0.710 min, (329.1 [M+H]⁺), 97.113% purity.

¹H NMR (400 MHz, CHCl₃-d): δ 8.68 (d, J=6.0 Hz, 2H), 8.03 (s, 1H), 7.36 (d, J=4.4 Hz, 2H), 6.46 (dd, J=10.4, 2.0 Hz, 1H), 6.31 (s, 1H), 4.20 (br. s, 2H), 4.08-4.04 (m, 1H), 3.80-3.77 (m, 1H), 2.68-2.66 (m, 1H), 2.16-2.13 (m, 2H), 1.79-1.74 (m, 2H), 1.63-1.60 (m, 1H) ppm.

### 7-methyl-6-(pyridin-4-yl)-1-(tetrahydro-2H-pyran-2-yl)-1H-indazol-4-amine 2.025

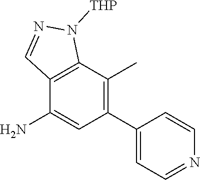

To a solution of compound 2.024 (240 mg, 729.94 μmol) and 2,4,6-trimethyl-1,3,5,2,4,6-trioxatriborinane (1.83 g, 7.30 mmol, 2.04 mL, 50% purity in THF) in DMF (5 mL) was added K₂CO₃ (302.65 mg, 2.19 mmol) and Pd(dppf)Cl₂ (53.41 mg, 72.99 μmol) under N₂ at 20° C. The mixture was stirred at 100° C. for 16 h. The mixture was cooled to 20° C. 2,4,6-trimethyl-1,3,5,2,4,6-trioxatriborinane (458.16 mg, 3.65 mmol, 510.20 μL, 50% purity in THF), K₂CO₃ (201.76 mg, 1.46 mmol) and Pd(dppf)Cl₂ (53.41 mg, 72.99 μmol) was added. The mixture was stirred at 100° C. for 16 h. Water (30 mL) was added and the mixture was extracted with EA (40 mL×3). The combined organic phase was washed (brine, 50 mL×2), dried (Na₂SO₄), filtered and concentrated in vacuo to give a residue, which was purified (PM37) to afford compound 2.025 (220 mg, 661.52 μmol, 90.63% yield) as a yellow solid.

LCMS (AM3): rt=0.651 min, (309.2[M+H]⁺), 90.630% purity.

¹H NMR (400 MHz, CHCl₃-d): δ 8.66-8.65 (m, 2H), 8.00 (s, 1H), 7.28-7.27 (m, 2H), 6.25 (s, 1H), 5.91 (dd, J=10.0, 1.6 Hz, 1H), 4.06-4.03 (m, 1H), 3.74-3.71 (m, 1H), 2.76-2.74 (m, 1H), 2.48 (s, 3H), 2.21-2.15 (m, 2H), 1.78-1.72 (m, 3H) ppm.

### tert-butyl (3-chloro-4-(trifluoromethoxy)benzyl)(3-((3-((7-methyl-6-(pyridin-4-yl)-1-(tetrahydro-2H-pyran-2-yl)-1H-indazol-4-yl)amino)propyl)amino)-3-oxopropyl)carbamate 2.026

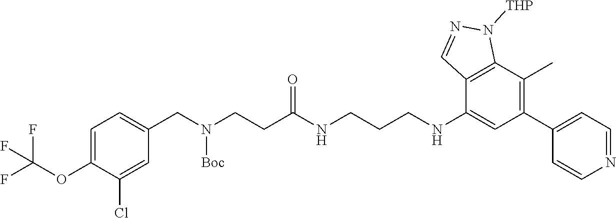

A solution of compound 1.325 (88.11 mg, 194.57 μmol) and compound 2.025 (50 mg, 162.14 μmol) in MeOH (0.5 mL) was stirred at 20° C. for 12 h. To the mixture was added NaBH₃CN (30.57 mg, 486.42 μmol) and the reaction was stirred at 20° C. for 2 h. The mixture was concentrated in vacuo to give a residue, which was purified (PM147) to afford compound 2.026 (50 mg, 58.21 μmol, 35.90% yield) as a yellow oil.

LCMS (AM7): rt=1.105 min, (745.3 [M+H]⁺), 86.753% purity.

### Synthesis of Intermediate 2.027

### tert-butyl (3-((3-((6-(6-aminopyridazin-4-yl)-1-(tetrahydro-2H-pyran-2-yl)-1H-indazol-4-yl) amino)propyl)amino)-3-oxopropyl)(3-chloro-4-(trifluoromethoxy)benzyl)carbamate 2.027

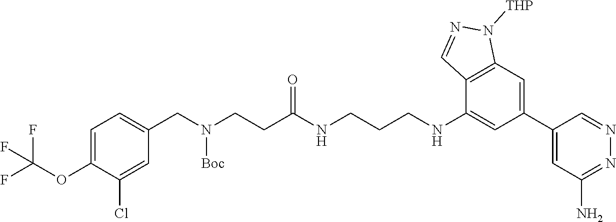

A solution of compound 2.016 (50 mg, 122.83 μmol, 76.244% purity) and compound 1.325 (66.75 mg, 147.40 μmol) in MeOH (0.5 mL) was stirred at 30° C. for 12 h. To the mixture was added NaBH₃CN (23.16 mg, 368.50 μmol) and the mixture was stirred at 30° C. for 2 h. Water (1 mL) was added and the mixture was purified (PM148) to afford compound 2.027 (40 mg, 46.52 μmol, 37.87% yield, 86.895% purity) as a yellow solid.

LCMS (AM7): rt=1.057 min, (747.1 [M+H]⁺), 86.895% purity.

### Synthesis of Intermediate 2.038

### tert-butyl (6-cyano-1-(tetrahydro-2H-pyran-2-yl)-1H-indazol-4-yl)carbamate 2.034

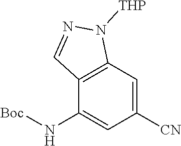

To a solution of compound 2.012 (800 mg, 3.30 mmol) and DMAP (40.34 mg, 330.20 μmol) in DCM (1 mL) was added (Boc)₂O (1.08 g, 4.95 mmol, 1.14 mL) at 0° C. The mixture was stirred at 20° C. for 1 h. The reaction mixture was concentrated in vacuo to remove solvent. Water (10 mL) was added and extracted with EA (30 mL×3). The combined organic layers were washed (brine, 50 mL×3), dried (Na₂SO₄), filtered and concentrated in vacuo to give a residue, which was purified (PM35) to afford compound 2.034 (600 mg, 1.70 mmol, 45.86% yield) as a yellow oil.

¹H NMR (400 MHz, MeOH-d₄) δ8.20-8.19 (m, 1H), 8.02-7.98 (m, 1H), 7.38 (d, J=0.8 Hz, 1H), 5.77-5.75 (m, 1H), 4.02-3.98 (m, 1H), 3.80-3.75 (m, 1H), 2.47-2.44 (m, 1H), 2.14-2.11 (m, 2H), 1.78-1.71 (m, 3H), 1.39 (s, 9H) ppm.

### tert-butyl (6-(N-hydroxycarbamimidoyl)-1-(tetrahydro-2H-pyran-2-yl)-1H-indazol-4-yl) carbamate 2.035

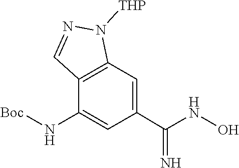

To a solution of NH₂OH·HCl (25.37 mg, 365.08 μmol) and KOAc (35.83 mg, 365.08 mol) in EtOH (1 mL) and H₂O (0.2 mL) was added compound 2.034 (100 mg, 292.06 mol) at 20° C. The mixture was stirred at 70° C. for 1 h. The mixture was concentrated in vacuo to give a residue, which was purified (PM143) to afford compound 2.035 (50 mg, 97.66 μmol, 26.75% yield, FA salt) as an off-white solid.

LCMS (AM3): rt=0.772 min, (375.9 [M+H]⁺), 82.318% purity.

¹H NMR (400 MHz, DMSO-d₆) δ 8.37 (s, 1H), 7.99 (s, 1H), 7.63 (s, 1H), 5.78-5.76 (m, 1H), 3.91-3.89 (m, 1H), 3.76-3.73 (m, 1H), 2.43-2.39 (m, 1H), 2.05-2.02 (m, 1H), 1.94-1.91 (m, 1H), 1.58-1.56 (m, 3H), 1.51 (s, 9H) ppm.

### tert-butyl (6-(1,2,4-oxadiazol-3-yl)-1-(tetrahydro-2H-pyran-2-yl)-1H-indazol-4-yl)carbamate 2.036

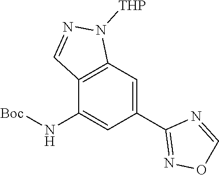

A solution of compound 2.035 (50 mg, 118.64 mol, FA salt) and trimethoxymethane (100.72 mg, 949.11 μmol, 104.05 L) in EtOH (2 mL) was stirred at 80° C. for 5 h. The mixture was concentrated in vacuo to give a residue, which was purified (PM38) to afford compound 2.036 (20 mg, 47.60 μmol, 40.12% yield) as a colourless oil.

LCMS (AM3): rt=0.856 min, (386.3 [M+H]⁺), 91.72% purity.

### 6-(1,2,4-oxadiazol-3-yl)-1H-indazol-4-amine 2.037

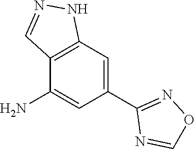 [SMILES: Nc1cc(-c2ncon2)cc2[nH]ncc12]

To a solution of compound 2.036 (20 mg, 51.89 μmol) in DCM (0.5 mL) was added TFA (770.00 mg, 6.75 mmol, 0.5 mL). The mixture was stirred at 20° C. for 1 h. The mixture was concentrated in vacuo to give a residue, which was adjusted to pH=8 with DIPEA and then purified (PM86) to afford compound 2.037 (3.41 mg, 16.81 μmol, 32.39% yield) as a yellow solid.

LCMS (AM3): rt=0.275 min, (202.0 [M+H]⁺), 99.16% purity.

¹H NMR (400 MHz, CHCl₃-d) δ 9.23 (s, 1H), 8.15 (s, 1H), 7.56 (s, 1H), 7.03 (s, 1H) ppm.

### tert-butyl (3-((3-((6-(1,2,4-oxadiazol-3-yl)-1H-indazol-4-yl)amino)propyl)amino)-3-oxopropyl) (3-chloro-4-(trifluoromethoxy)benzyl)carbamate 2.038

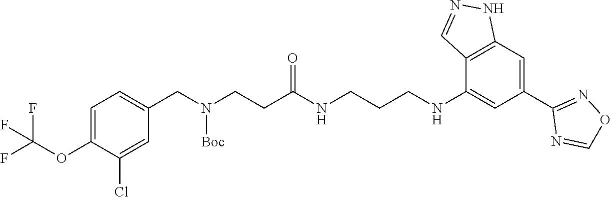 [SMILES: CC(C)(C)OC(=O)N(CCC(=O)NCCCNc1cc(-c2ncon2)cc2[nH]ncc12)Cc1ccc(OC(F)(F)F)c(Cl)c1]

A solution of compound 2.037 (50 mg, 185.31 μmol, 74.564% purity) and compound 1.325 (83.92 mg, 185.31 μmol) in MeOH (0.5 mL) was stirred at 30° C. for 12 h. To the mixture was added NaBH₃CN (34.94 mg, 555.94 μmol) and the mixture was stirred at 30° C. for 2 h. The mixture was diluted with MeOH (1 mL) and purified (PM149) to afford compound 2.038 (50 mg, 68.37 μmol, 36.90% yield) as a yellow oil

LCMS (AM7): rt=1.052 min, (638.0 [M+H]⁺), 87.25% purity.

### Synthesis of Intermediate 2.045

### 6-bromo-4-fluoro-1H-benzo[d][1,2,3]triazole 2.040

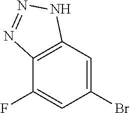 [SMILES: Fc1cc(Br)cc2[nH]nnc12]

To a solution of 5-Bromo-3-fluorobenzene-1,2-diamine (CAS 517920-69-3, 9.3 g, 45.36 mmol) in AcOH (35 mL) and H₂O (100 mL) was added a solution of NaNO₂ (3.44 g, 49.90 mmol) at 20° C., then the mixture was stirred at 65° C. for 1 h. The reaction mixture was adjusted to pH=8-9 by saturated NaHCO₃ solution and extracted with EA (150 mL×2). The combined organic layers were washed (brine, 100 mL×2), dried (Na₂SO₄), filtered and concentrated in vacuo to afford compound 2.040 (9.7 g, 44.91 mmol, 99.00% yield) as a black-brown solid, which was used for the next step directly without further purification.

LCMS (AM3): rt=0.794 min, (218.1 [M+H]⁺), 100% purity

### 6-bromo-4-fluoro-1-(tetrahydro-2H-pyran-2-yl)-1H-benzo[d][1,2,3]triazole 2.041

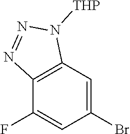

To a solution of DHP (18.89 g, 224.55 mmol, 20.53 mL) and compound 2.040 (9.7 g, 44.91 mmol) in DCM (150 mL) was added TsOH (773.36 mg, 4.49 mmol). The mixture was stirred at 20° C. for 20 h. The mixture was concentrated in vacuo to give a residue, which was purified (PM59) to afford compound 2.041 (11.4 g, 37.98 mmol, 84.58% yield) as a light-yellow solid.

¹H NMR (400 MHz, DMSO) δ 8.12 (s, 1H), 7.61 (d, J=10.0 Hz, 1H), 6.21-6.17 (m, 1H), 2.50-2.43 (m, 2H), 2.20-2.02 (m, 2H), 1.69-1.65 (m, 2H), 1.65-1.61 (m, 2H).

### 4-fluoro-1-(tetrahydro-2H-pyran-2-yl)-1H-benzo[d][1,2,3]triazol-6-amine 2.042

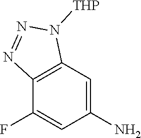

To a solution of compound 2.041 (5.7 g, 18.99 mmol) in DMSO (75 mL) was added NH₃·H₂O (7.99 g, 56.98 mmol, 8.78 mL, 25% purity), K₂CO₃ (7.87 g, 56.98 mmol), CuI (723.40 mg, 3.80 mmol) and (2R,4R)-4-hydroxypyrrolidine-2-carboxylic acid (996.17 mg, 7.60 mmol). The reaction mixture was stirred at 90° C. for 12 h under N₂. The reaction mixture was diluted with H₂O (60 mL) and extracted with EA (80 mL×2). The combined organic layer was washed (brine, 100 mL), dried (Na₂SO₄), filtered and concentrated in vacuo to give a residue, which was purified (PM25) to afford compound 2.042 (2.5 g, 10.58 mmol, 55.72% yield) as a yellow oil.

LCMS (AM3): rt=0.720 min, (237.1 [M+H]⁺), 75.30% purity

### 4-fluoro-1-(tetrahydro-2H-pyran-2-yl)-6-(4H-1,2,4-triazol-4-yl)-1H-benzo[d][1,2,3]triazole 2.043

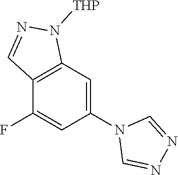

To a solution of N′-formylformohydrazide (4.66 g, 52.91 mmol) and compound 2.042 (2.5 g, 10.58 mmol) in pyridine (20 mL) was added TEA (7.50 g, 74.08 mmol, 10.31 mL) and TMSCl (17.24 g, 158.73 mmol, 20.15 mL). The mixture was stirred at 120° C. for 16 h. The reaction mixture was diluted with water (100 mL) and extracted with EA (100 mL×2). The combined organic layers were washed (brine, 50 mL×2), dried (Na₂SO₄), filtered and concentrated in vacuo to give a residue, which was purified (PM23) to afford compound 2.043 (1.35 g, 4.68 mmol, 44.25% yield) as a light-yellow solid LCMS (AM3): rt=0.794 min, (289.2 [M+H]⁺), 93.9% purity

### tert-butyl (4-(2-((1-(tetrahydro-2H-pyran-2-yl)-6-(4H-1,2,4-triazol-4-yl)-1H-benzo[d][1,2,3]triazol-4-yl)amino)ethoxy)butyl)carbamate 2.044

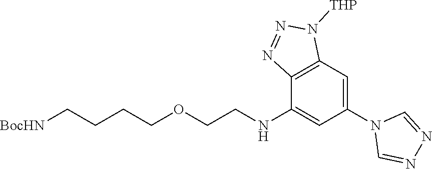

To a solution of compound 1.461 (1.45 g, 6.24 mmol) and compound 2.043 (0.9 g, 3.12 mmol) in NMP (10 mL) was added DIPEA (806.98 mg, 6.24 mmol, 1.09 mL), then the mixture was stirred at 130° C. for 18 h. The reaction mixture was diluted with water (50 mL) and extracted with EA (75 mL×2). The combined organic layers were washed (brine, 50 mL×2), dried (Na₂SO₄), filtered and concentrated in vacuo to give a residue, which was purified (PM23) to afford compound 2.044 (1.50 g, 3.00 mmol, 95.98% yield) as a light-yellow oil.

LCMS (AM3): rt=0.865 min, (501.2 [M+H]⁺), 75.58% purity

### N-(2-(4-aminobutoxy)ethyl)-6-(4H-1,2,4-triazol-4-yl)-1H-benzo[d][1,2,3]triazol-4-amine 2.045

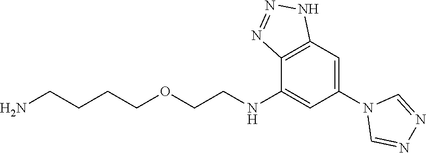 [SMILES: NCCCCOCCNc1cc(-n2cnnc2)cc2[nH]nnc12]

To a solution of compound 2.044 (1.82 g, 3.64 mmol) in EA (7.5 mL) was added HCl/EA (4 M, 7.46 mL). The mixture was stirred at 0-5° C. for 2 h. The reaction mixture was concentrated in vacuo to give a residue, which was triturated with EA at 20° C. for 15 min and filtered. The filter cake was dried in vacuo to afford compound 2.045 (1.55 g, 3.64 mmol, 100.00% yield, HCl salt) as a yellow solid.

LCMS (AM3): rt=0.250 min, (317.2 [M+H]⁺), 96.98% purity

### Synthesis of Intermediate 2.064

### tert-butyl (4-(2-((6-bromo-1-(tetrahydro-2H-pyran-2-yl)-1H-benzo[d][1,2,3]triazol-4-yl)amino)ethoxy)butyl)carbamate 2.062

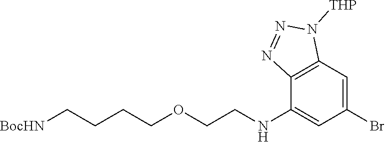

To a solution of tert-Butyl (4-(2-aminoethoxy)butyl)carbamate (CAS 2830620-37-4, 899.86 mg, 3.87 mmol) and compound 2.041 (775 mg, 2.58 mmol) in NMP (10 mL) was added DIPEA (667.47 mg, 5.16 mmol, 899.56 L). The mixture was stirred at 140° C. for 18 h. The reaction mixture was diluted with water (50 mL) and extracted with EA (50 mL×2). The combined organic layers were washed (brine, 30 mL×2), dried (Na₂SO₄), filtered and concentrated in vacuo to give a residue, which was purified (PM55) to afford compound 2.062 (640 mg, 1.25 mmol, 48.37% yield) as a light-yellow oil.

LCMS (AM3): rt=1.066 min, (512.2 [M+H]⁺), 88.9% purity

### tert-butyl (4-(2-((6-(isoxazol-4-yl)-1-(tetrahydro-2H-pyran-2-yl)-1H-benzo[d][1,2,3]triazol-4-yl)amino)ethoxy)butyl)carbamate 2.063

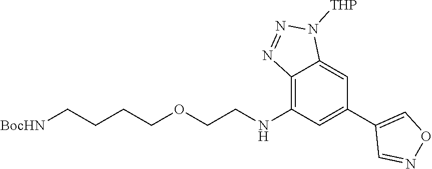

To a mixture of compound 2.062 (640 mg, 1.25 mmol) and isoxazol-4-ylboronic acid (292.28 mg, 1.50 mmol) in DMF (9 mL) and H₂O (3.75 mL) was added KF (217.69 mg, 3.75 mmol, 87.78 L) and Pd(dppf)Cl₂ (91.39 mg, 124.89 μmol). The mixture was stirred at 60° C. for 2.5 h under N₂. The reaction mixture was diluted with water (50 mL) and extracted with EA (75 mL×2). The combined organic layers were washed (brine, 50 mL×2), dried (Na₂SO₄), filtered and concentrated in vacuo to give a residue, which was purified (PM35) to afford compound 2.063 (370 mg, 739.13 μmol, 59.18% yield) as a brown oil.

LCMS (AM3): rt=0.947 min, (501.2 [M+H]⁺), 100% purity

### N-(2-(4-aminobutoxy)ethyl)-6-(isoxazol-4-yl)-1H-benzo[d][1,2,3]triazol-4-amine 2.064

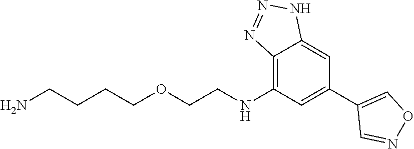 [SMILES: NCCCCOCCNc1cc(-c2cnoc2)cc2[nH]nnc12]

To a solution of compound 2.063 (720 mg, 1.44 mmol) in EA (5 mL) was added HCl/EA (4 M, 5 mL) at 0° C. The mixture was stirred at 0° C. for 1 h. The mixture was filtered and the filter cake was dried in vacuo to afford compound 2.064 (320 mg, 822.03 μmol, 57.15% yield, HCl salt) as a yellow solid.

LCMS (AM3): rt=0.666 min, (317.2 [M+H]⁺), 93.82% purity

### Synthesis of Intermediate 2.067

### tert-butyl(4-(2-((1-(tetrahydro-2H-pyran-2-yl)-6-(4,4,5,5-tetramethyl-1,3,2-dioxaborolan-2-yl)-1H-benzo[d][1,2,3]triazol-4-yl)amino)ethoxy)butyl)carbamate 2.065

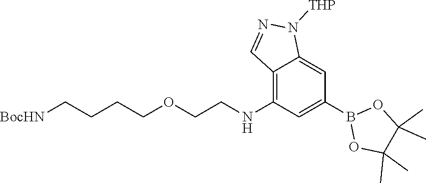

To a mixture of compound 2.062 (1.5 g, 2.93 mmol) and B₂pin₂ (891.98 mg, 3.51 mmol) in 1,4-dioxane (15 mL) was added KOAc (574.55 mg, 5.85 mmol) and Pd(dppf)Cl₂ (214.18 mg, 292.72 μmol), then the mixture was stirred at 85° C. for 16 h under N₂. The reaction mixture was diluted with water (30 mL) and extracted with EA (50 mL×2). The combined organic layers were washed (brine, 300 mL×2), dried (Na₂SO₄), filtered and concentrated in vacuo to give a residue, which was purified (PM55) to afford compound 2.065 (1.2 g, 2.14 mmol, 73.27% yield) as a brown oil.

LCMS (AM3): rt=1.102 min, (560.6 [M+H]⁺), 77.5% purity

### tert-butyl(4-(2-((6-(pyridazin-4-yl)-1-(tetrahydro-2H-pyran-2-yl)-1H-benzo[d][1,2,3]triazol-4-yl)amino)ethoxy)butyl)carbamate 2.066

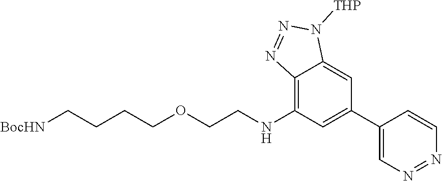

To a mixture of compound 2.065 (1.15 g, 2.06 mmol) and 4-bromopyridazine (450 mg, 1.88 mmol, HBr salt) in 1,4-dioxane (13.5 mL) and H₂O (1.5 mL) was added K₂CO₃ (777.77 mg, 5.63 mmol) and Pd(dppf)Cl₂ (137.26 mg, 187.58 μmol), then the mixture was stirred at 85° C. for 16 h under N₂. The reaction mixture was diluted with water (50 mL) and extracted with EA (75 mL×2). The combined organic layers were washed (brine, 50 mL×2), dried (Na₂SO₄), filtered and concentrated in vacuo to give a residue, which was purified (PM23) to afford compound 2.066 (710 mg, 1.39 mmol, 73.98% yield) as a brown oil LCMS (AM3): rt=0.950 min, (512.4 [M+H]⁺), 93.32% purity

### N-(2-(4-aminobutoxy)ethyl)-6-(pyridazin-4-yl)-1H-benzo[d][1,2,3]triazol-4-amine 2.067

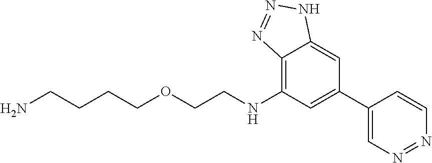 [SMILES: NCCCCOCCNc1cc(-c2ccnnc2)cc2[nH]nnc12]

To a solution of compound 2.066 (788 mg, 1.54 mmol) in EA (5 mL) was added HCl/EA (4 M, 5 mL) at 0-5° C., then the mixture was stirred at 0-5° C. for 2 h. The mixture was filtered and filter cake was dried in vacuo to afford compound 2.067 (670 mg, 1.53 mmol, 99.60% yield, HCl salt) as a brown solid.

LMS (AM3): rt=0.662 min, (328.1 [M+H]⁺), 86.3% purity

### Synthesis of Intermediate 2.069

### tert-butyl (3-chloro-4-(trifluoromethoxy)benzyl)(2-(5-(2-oxo-2-((1-(tetrahydro-2H-pyran-2-yl)-6-(4H-1,2,4-triazol-4-yl)-1H-indazol-4-yl)amino)ethyl)oxazol-2-yl)ethyl)carbamate 2.068

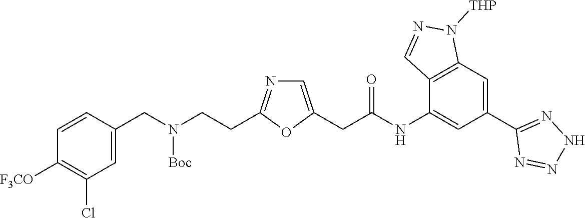

To a mixture of compound 1.246 (178.13 mg, 0.626 mmol) and compound 1.273 (300 mg, 0.626 mmol) in THF (5 mL) were added DIPEA (242.91 mg, 1.88 mmol) and T3P (598.03 mg, 0.939 mmol). The mixture was stirred at 20° C. for 12 h. The mixture was diluted with water (30 mL) and extracted with EA (30 mL×3). The combined organic phase was washed (brine, 90 mL), dried (Na₂SO₄), filtered and concentrated in vacuo to give a residue, which was purified (PM150) to afford compound 2.068 (80 mg, 107.36 μmol, 17.14% yield) as a white solid.

LCMS (AM3): rt=1.027 min, (745.3 [M+H]⁺),100% purity.

### tert-butyl (3-chloro-4-(trifluoromethoxy)benzyl)(2-(5-(2-((1-(tetrahydro-2H-pyran-2-yl)-6-(4H-1,2,4-triazol-4-yl)-1H-indazol-4-yl)amino)ethyl)oxazol-2-yl)ethyl)carbamate 2.069

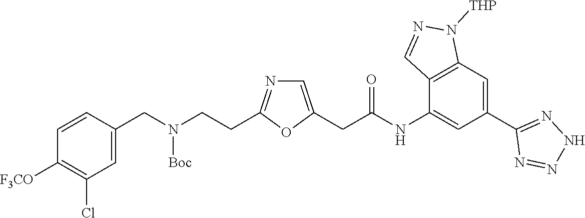

To a mixture of compound 2.068 (80 mg, 107.36 μmol) in THF (2 mL) was added BH₃-Me₂S (10 M, 0.05 mL) at 0° C. The mixture was stirred at 20° C. for 36 h. The mixture was quenched with MeOH (5 mL) and followed by NaOH (1N, 5 mL). The mixture was stirred at 60° C. for 4 h, then the mixture was extracted with EA (20 mL×3). The combined organic phase was washed (brine, 50 mL), dried (Na₂SO₄), filtered and concentrated in vacuo. The residue was purified (PM151) to afford compound 2.069 (50 mg, 68.38 μmol, 63.70% yield) as a grey solid.

LCMS (AM3): rt=0.862 min, (731.1 [M+H]⁺),94.9% purity.

### Synthesis of Intermediate 2.071

### 6-(3-methyl-4H-1,2,4-triazol-4-yl)-4-nitro-1H-indazole 2.070

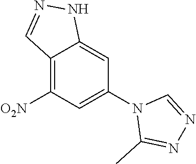 [SMILES: Cc1nncn1-c1cc([N+](=O)[O-])c2cn[nH]c2c1]

To a mixture of compound 1.271 (203.38 mg, 2.75 mmol) in ACN (3 mL) was added DMF-DMA (327.14 mg, 2.75 mmol). The mixture was stirred at 50° C. for 0.5 h, then acetohydrazide (600 mg, 2.29 mmol) and HOAc (6.30 g, 104.91 mmol) were added. The mixture was stirred at 120° C. for 16 h. The mixture was adjusted to pH=8 by saturated Na₂CO₃ solution and extracted with EA (20 mL×3). The combined organic phase was washed (brine, 50 mL), dried (Na₂SO₄), filtered and concentrated in vacuo to give a residue, which was purified (PM22) to afford compound 2.070 (120 mg, 491.38 μmol, 21.48% yield) as a yellow solid.

LCMS (AM3): rt=0.485 min, (244.8 [M+H]⁺), 90.5% purity.

### 6-(3-methyl-4H-1,2,4-triazol-4-yl)-1H-indazol-4-amine 2.071

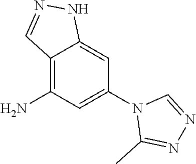 [SMILES: Cc1nncn1-c1cc(N)c2cn[nH]c2c1]

To a solution of compound 2.070 (120 mg, 491.38 μmol) in MeOH (5 mL) was added Pd/C (30 mg, 10% purity) under N₂. The suspension was degassed and purged with H₂ (×3). The mixture was stirred at 30° C. for 4 h under H₂ (15 psi). The catalyst was filtered and the filtrate was concentrated in vacuo to afford compound 2.071 (100 mg, crude) as a white solid, which was used directly for the next step.

LCMS (AM5): rt=0.265 min, (215.2 [M+H]⁺), 85.16% purity.

### Synthesis of Intermediate 2.081

### 5-bromo-1-(4-methoxybenzyl)-7-nitro-1H-indazole 2.074

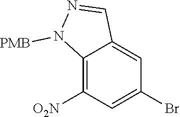

To a mixture of 5-Bromo-7-nitro-1H-indazole (CAS 316810-82-9, 2 g, 8.26 mmol) and K₂CO₃ (1.71 g, 12.40 mmol) in DMF (20 mL) was added PMB-CI (1.55 g, 9.92 mmol) at 0° C. The mixture was stirred at 25° C. for 12 h. The mixture was diluted with water (20 mL) and extracted with EA (20 mL×3). The combined organic phase was washed (brine, 50 mL×3), dried (Na₂SO₄), filtered and concentrated in vacuo to give a residue, which was purified (PM51) to afford compound 2.074 (2 g, 5.52 mmol, 66.83% yield) as a yellow solid.

¹H NMR (400 MHz, DMSO-d₆) δ 8.54 (d, J=1.6 Hz, 1H), 8.48 (s, 1H), 8.23 (d, J=1.6 Hz, 1H), 6.89-6.77 (m, 4H), 5.68 (s, 2H), 3.67 (s, 3H) ppm

### 1-(4-methoxybenzyl)-7-nitro-1H-indazol-5-amine 2.075

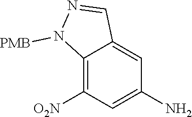

To a mixture of compound 2.074 (2 g, 5.52 mmol) in DMSO (20 mL) were added NH₃·H₂O (9.10 g, 64.91 mmol, 10 mL, 25% purity), CuI (220.86 mg, 1.16 mmol), (2S,4S)-4-hydroxypyrrolidine-2-carboxylic acid (296.89 mg, 2.26 mmol) and K₂CO₃ (2.29 g, 16.57 mmol). The mixture was stirred at 90° C. for 16 h under N₂. The mixture was diluted with water (40 mL) and extracted with EA (40 mL×3). The combined organic phase was washed (brine, 100 mL), dried (Na₂SO₄), filtered and concentrated in vacuo to give a residue, which was purified (PM38) to afford compound 2.075 (500 mg, 1.59 mmol, 28.84% yield) as a yellow oil.

LCMS (AM3): rt=0.849 min, (299.2 [M+H]⁺), 95% purity.

### 1-(4-methoxybenzyl)-7-nitro-5-(4H-1,2,4-triazol-4-yl)-1H-indazole 2.076

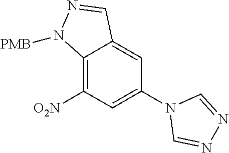

To a mixture of N′-formylformohydrazide (738.07 mg, 8.38 mmol), compound 2.075 (500 mg, 1.68 mmol) and Et₃N (1.19 g, 11.73 mmol) in pyridine (7 mL) was added TMSCl (2.73 g, 25.14 mmol). The mixture was stirred at 120° C. for 12 h under N₂. The mixture was concentrated in vacuo to give a residue, which was purified (PM90) to afford compound 2.076 (380 mg, 1.08 mmol, 64.71% yield) as a yellow solid.

LCMS (AM3): rt=0.862 min, (351.2 [M+H]⁺), 99.6% purity.

### 1-(4-methoxybenzyl)-5-(4H-1,2,4-triazol-4-yl)-1H-indazol-7-amine 2.077

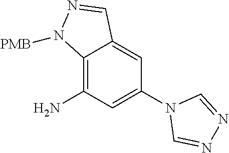

To a solution of compound 2.076 (380 mg, 1.08 mmol) in MeOH (5 mL) was added Pd/C (50 mg, 1.08 mmol, 10% purity) under N₂. The suspension was degassed and purged with H₂ (×3). The mixture was stirred at 30° C. for 16 h under H₂ (15 psi). The catalyst was filtered and the filtrate was concentrated in vacuo to afford compound 2.077 (350 mg, crude) as a white solid.

LCMS (AM3): rt=0.703 min, (321.2 [M+H]⁺), 98% purity.

### tert-butyl(3-((1-(4-methoxybenzyl)-5-(4H-1,2,4-triazol-4-yl)-1H-indazol-7-yl)amino)-3-oxopropyl)carbamate 2.078

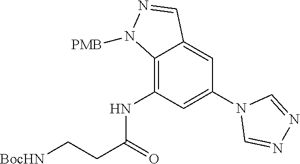

To a mixture of compound 2.077 (350 mg, 1.09 mmol) and 3-((tert-butoxycarbonyl)amino)propanoic acid (1.03 g, 5.46 mmol) in pyridine (10 mL) was added EDCI (2.09 g, 10.93 mmol). The mixture was stirred at 110° C. for 16 h. The mixture was concentrated in vacuo to give a residue, which was purified (PM153) to afford compound 2.078 (260 mg, 528.95 μmol, 48.41% yield) as a yellow solid.

LCMS (AM3): rt=0.866 min, (492.2 [M+H]⁺), 96.8% purity.

### tert-butyl(3-((1-(4-methoxybenzyl)-5-(4H-1,2,4-triazol-4-yl)-1H-indazol-7-yl)amino)propyl)carbamate 2.079

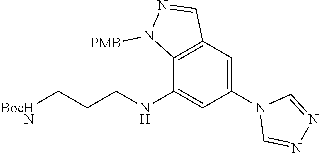

To a mixture of compound 2.078 (200 mg, 406.88 μmol) in THF (5 mL) was added BH₃-Me₂S (10 M, 0.45 mL) at 0° C. The mixture was stirred at 30° C. for 16 h. The mixture was quenched with MeOH (10 mL) and stirred at 65° C. for 16 h. The mixture was concentrated in vacuo to give a residue, which was purified (PM155) to afford compound 2.079 (20 mg, 41.88 μmol, 10.29% yield) as a brown oil.

LCMS (AM3): rt=0.929 min, (478.2 [M+H]⁺), 98.7% purity.

### N1-(1-(4-methoxybenzyl)-5-(4H-1,2,4-triazol-4-yl)-1H-indazol-7-yl)propane-1,3-diamine 2.080

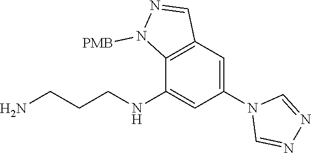

A mixture of compound 2.079 (20 mg, 41.88 μmol) in HCl/MeOH (4 M, 2 mL) was stirred at 30° C. for 1 h. The mixture was concentrated in vacuo to afford compound 2.080 (17 mg, crude, HCl salt) as a brown solid, which was used directly for the next step.

LCMS (AM3): rt=0.735 min, (378.2 [M+H]⁺), 97.8% purity.

### tert-butyl3-chloro-4-(trifluoromethoxy)benzyl(3-((3-((1-(4-methoxybenzyl)-5-(4H-1,2,4-triazol-4-yl)-1H-indazol-7-yl)amino)propyl)amino)-3-oxopropyl)carbamate 2.081

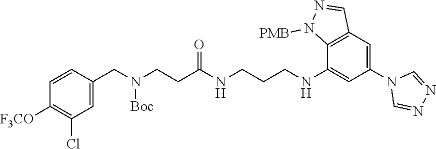

To a mixture of compound 2.080 (17 mg, 41.07 μmol, HCl salt) and compound 1.243 (17.92 mg, 45.04 μmol) in DMF (1 mL) were added HATU (20.55 mg, 54.05 μmol) and DIPEA (17.46 mg, 135.12 μmol). The mixture was stirred at 30° C. for 2 h. The mixture was diluted with water (20 mL) and extracted with EA (10 mL×3). The combined organic phase was washed (brine, 20 mL), dried (Na₂SO₄), filtered and concentrated in vacuo to give a residue, which was purified (PM121) to afford compound 2.081 (40 mg, crude) as a yellow oil.

LCMS (AM3): rt=1.027 min, (757.3 [M+H]⁺), 92.56% purity.

### Synthesis of Intermediate 2.086

### 4-bromo-3-methoxy-1H-pyrazole 2.083

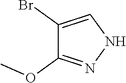

To a mixture of 5-Methoxy-1H-pyrazole (CAS 215610-30-3, 900 mg, 9.17 mmol) in ACN (10 mL) was added NBS (1.96 g, 11.01 mmol). The mixture was stirred at RT for 12 h. The mixture was concentrated in vacuo to give a residue, which was purified (PM87) to afford compound 2.083 (1.2 g, 6.78 mmol, 73.90% yield) as a white solid.

LCMS (AM3): rt=0.502 min, (177.0 [M+H]⁺), 99% purity.

### 4-bromo-3-methoxy-1-(tetrahydro-2H-pyran-2-yl)-1H-pyrazole 2.084

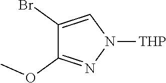

To a mixture of compound 2.083 (1.2 g, 6.78 mmol) and DHP (920.00 mg, 10.94 mmol) in THF (10 mL) was added TsOH·H₂O (644.81 mg, 3.39 mmol). The mixture was stirred at 60° C. for 12 h. The mixture was concentrated in vacuo to give a residue, which was purified (PM88) to afford compound 2.084 (1.3 g, 4.98 mmol, 73.43% yield) as a yellow oil.

¹H NMR (400 MHz, MeOH-d₄) δ 7.70 (s, 1H), 5.19 (dd, J=10.0, 2.4 Hz, 1H), 4.07-3.97 (m, 1H), 3.92 (s, 3H), 3.76-3.63 (m, 1H), 2.20-1.89 (m, 3H), 1.80-1.58 (m, 3H) ppm.

### tert-butyl(4-(2-((6-(3-methoxy-1-(tetrahydro-2H-pyran-2-yl)-1H-pyrazol-4-yl)-1-(tetrahydro-2H-pyran-2-yl)-1H-indazol-4-yl)amino)ethoxy)butyl)carbamate 2.085

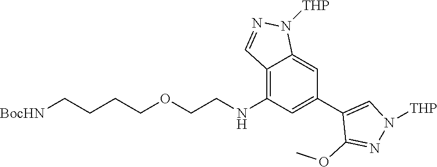

To a mixture of compound 2.084 (280.51 mg, 1.07 mmol) and compound 1.919 (300 mg, 537.14 μmol) in dioxane (10 mL) and H₂O (1 mL) were added BrettPhos Pd G3 (48.69 mg, 53.71 μmol) and K₂CO₃ (148.47 mg, 1.07 mmol). The mixture was stirred at 80° C. for 12 h under N₂. The mixture was concentrated in vacuo to give a residue, which was purified (PM35) to afford compound 2.085 (270 mg, crude) as a yellow oil.

LCMS (AM3): rt=1.009 min, (613.4 [M+H]⁺), 64% purity.

### N-(2-(4-aminobutoxy)ethyl)-6-(3-methoxy-1H-pyrazol-4-yl)-1H-indazol-4-amine 2.086

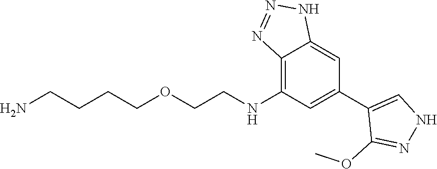 [SMILES: COc1n[nH]cc1-c1cc(NCCOCCCCN)c2nn[nH]c2c1]

A mixture of compound 2.085 (260 mg, 424.31 μmol) in HCl/dioxane (4 M, 5 mL) was stirred at 20° C. for 0.5 h. The mixture was concentrated in vacuo to give a residue, which was purified (PM89) to afford compound 2.086 (160 mg, 420.09 μmol, 99.01% yield, HCl salt) as a brown solid.

LCMS (AM5): rt=0.789 min, (345.4 [M+H]⁺), 82% purity.

### Synthesis of Intermediate 2.093

### 6-(3-methyl-4H-1,2,4-triazol-4-yl)-4-nitro-1-(tetrahydro-2H-pyran-2-yl)-1H-indazole 2.089

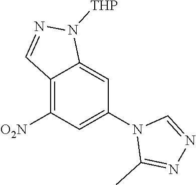

To a mixture of acetohydrazide (406.76 mg, 5.49 mmol) in ACN (5 mL) was added DMF-DMA (654.28 mg, 5.49 mmol). The mixture was stirred at 50° C. for 0.5 h, then compound 1.271 (1.2 g, 4.58 mmol) and AcOH (10.50 g, 174.85 mmol) was added. The mixture was stirred at 120° C. for 12 h. The mixture was adjusted to pH=8 by saturated Na₂CO₃solution and extracted with EA (20 mL×3). The combined organic phase was washed (brine, 50 mL), dried (Na₂SO₄), filtered and concentrated in vacuo to give a residue, which was purified (PM22) to afford compound 2.089 (360 mg, 1.10 mmol, 23.96% yield) as a brown oil.

LCMS (AM3): rt=0.789 min, (329.1 [M+H]⁺), 47.4% purity.

### 6-(3-methyl-4H-1,2,4-triazol-4-yl)-1-(tetrahydro-2H-pyran-2-yl)-1H-indazol-4-amine 2.090

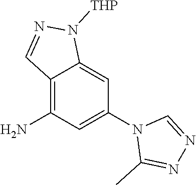

To a solution of compound 2.089 (360 mg, 1.10 mmol) in MeOH (10 mL) was added Pd/C (80 mg, 10% purity) under N₂. The suspension was degassed and purged with H₂ (×3). The mixture was stirred at 30° C. for 12 h under H₂ (15 psi). The catalyst was filtered and the filtrate was concentrated in vacuo to afford compound 2.090 (320 mg, crude) as a yellow oil, which was used directly for the next step.

LCMS (AM5): rt=0.842 min, (299.6 [M+H]⁺), 45.3% purity.

### tert-butyl(4-(2-((6-(3-methyl-4H-1,2,4-triazol-4-yl)-1-(tetrahydro-2H-pyran-2-yl)-1H-indazol-4-yl)amino)-2-oxoethoxy)butyl)carbamate 2.091

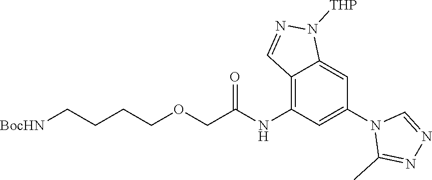

To a mixture of compound 2.090 (270 mg, 905.00 μmol) and compound 1.891 (447.59 mg, 1.81 mmol) in pyridine (5 mL) was added EDCI (693.96 mg, 3.62 mmol). The mixture was stirred at 80° C. for 12 h. The mixture was concentrated in vacuo to give a residue, which was purified (PM88) to afford compound 2.091 (250 mg, crude) as a pink solid.

LCMS (AM3): rt=0.893 min, (528.3 [M+H]⁺), 49% purity.

### tert-butyl(4-(2-((6-(3-methyl-4H-1,2,4-triazol-4-yl)-1-(tetrahydro-2H-pyran-2-yl)-1H-indazol-4-yl)amino)ethoxy)butyl)carbamate 2.092

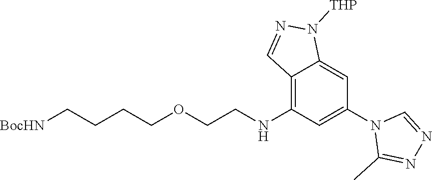

To a mixture of compound 2.091 (150 mg, 284.30 μmol) in THF (3 mL) was added BH₃-Me₂S (10 M, 284.30 μL) at 0° C. The mixture was stirred at 30° C. for 3 h. The mixture was quenched with MeOH (5 mL) and followed by NaOH (1N, 3 mL). The mixture was stirred at 60° C. for 3 h. The mixture was concentrated in vacuo to give a residue, which was purified (PM122) to afford compound 2.092 (85 mg, crude) as a yellow oil.

LCMS (AM3): rt=0.907 min, (514.3 [M+H]⁺), 96% purity.

### N-(2-(4-aminobutoxy)ethyl)-6-(3-methyl-4H-1,2,4-triazol-4-yl)-1H-indazol-4-amine 2.093

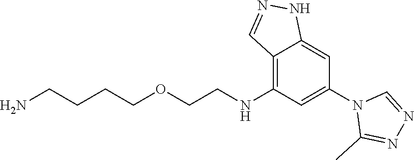 [SMILES: Cc1nncn1-c1cc(NCCOCCCCN)c2cn[nH]c2c1]

A mixture of compound 2.092 (85 mg, 165.49 μmol) in HCl/dioxane (4 M, 3 mL) was stirred at 25° C. for 1 h. The mixture was concentrated in vacuo to afford compound 2.093 (60 mg, crude, HCl salt) as a yellow oil, which was used directly for the next step.

LCMS (AM5): rt=0.683 min, (330.3 [M+H]⁺), 70% purity.

### Synthesis of Intermediate 2.095

### 4-(4-nitro-1-(tetrahydro-2H-pyran-2-yl)-1H-indazol-6-yl)picolinonitrile 2.094

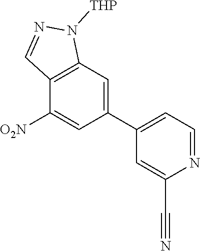

To a mixture of compound 1.943 (400 mg, 989.46 μmol) and 4-bromopicolinonitrile (217.29 mg, 1.19 mmol) in dioxane (2 mL) and H₂O (0.2 mL) was added K₂CO₃ (273.51 mg, 1.98 mmol) and Pd(dppf)Cl₂·CH₂Cl₂ (80.80 mg, 98.95 μmol) under N₂. The mixture was stirred at 90° C. for 12 h. The mixture was filtered and the filtrate was concentrated in vacuo to give a residue, which was purified (PM50) to afford compound 2.094 (350 mg, 811.52 μmol, 82.02% yield) as a yellow solid.

LCMS (AM3): rt=0.965 min, (350.5 [M+H]⁺), 81.7% purity

### 4-(4-amino-1-(tetrahydro-2H-pyran-2-yl)-1H-indazol-6-yl)picolinonitrile 2.095

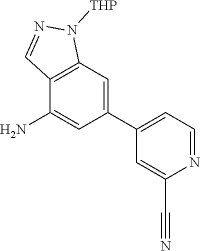

To a mixture of compound 2.094 (200 mg, 463.73 μmol) and NH₄Cl (248.05 mg, 4.64 mmol) in EtOH (5 mL) and H₂O (1 mL) was added Fe (51.79 mg, 927.46 μmol) under N₂. The mixture was stirred at 65° C. for 1 h. The mixture was filtered and the filtrate was concentrated in vacuo to afford compound 2.095 (150 mg, crude) as a yellow solid, which was used without further purification.

LCMS (AM3): rt=0.871 min, (320.2 [M+H]⁺), 79.7% purity

### Synthesis of Intermediate 2.098

### 4-nitro-6-(pyrimidin-4-yl)-1-(tetrahydro-2H-pyran-2-yl)-1H-indazole 2.097

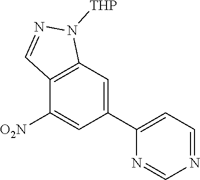

To a mixture of compound 1.943 (900 mg, 1.45 mmol) and 4-chloropyrimidine (263.32 mg, 1.74 mmol) in dioxane (2 mL) and H₂O (0.2 mL) was added K₂CO₃ (401.71 mg, 2.91 mmol) and Pd(dppf)Cl₂·CH₂Cl₂ (118.68 mg, 145.33 μmol) under N₂. The mixture was stirred at 90° C. for 12 h. The mixture was filtered and the filtrate was concentrated in vacuo to give a residue, which was purified (PM50) to afford compound 2.097 (240 mg, 442.64 μmol, 30.46% yield) as a brown solid.

LCMS (AM3): rt=0.738 min, (325.9 [M+H]⁺), 60.0% purity

### 6-(pyrimidin-4-yl)-1-(tetrahydro-2H-pyran-2-yl)-1H-indazol-4-amine 2.098

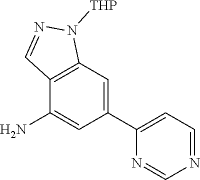

To a mixture of compound 2.097 (230 mg, 424.20 μmol) and NH₄Cl (226.90 mg, 4.24 mmol) in EtOH (5 mL) and H₂O (1 mL) was added Fe (47.38 mg, 848.39 μmol) under N₂. The mixture was stirred at 65° C. for 1 h. The mixture was filtered and the filtrate was concentrated in vacuo to afford compound 2.098 (150 mg, crude) as a yellow oil, which was used without further purification.

LCMS (AM3): rt=0.678 min, (296.1 [M+H]⁺), 75.2% purity

### Synthesis of Intermediate 2.100

### 6-(3-methoxy-1-(tetrahydro-2H-pyran-2-yl)-1H-pyrazol-4-yl)-1-(tetrahydro-2H-pyran-2-yl)-1H-indazol-4-amine 2.100

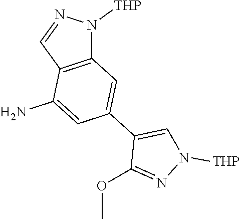

To a mixture of compound 2.084 (200 mg, 766.04 μmol) and compound 1.952 (240.03 mg, 919.24 μmol) in dioxane (2 mL) and H₂O (0.2 mL) was added K₂CO₃ (211.75 mg, 1.53 mmol) and Pd(dppf)Cl₂·CH₂Cl₂ (62.56 mg, 76.60 μmol) under N₂. The mixture was stirred at 90° C. for 12 h. The mixture was filtered and the filtrate was concentrated in vacuo to give a residue, which was purified (PM50) to afford compound 2.100 (170 mg, 402.04 μmol, 52.48% yield) as a yellow oil.

LCMS (AM3): rt=0.767 min, (398.1 [M+H]⁺), 74.9% purity

### Synthesis of Intermediate 2.103

### methyl 4-bromo-1-(tetrahydro-2H-pyran-2-yl)-1H-indazole-6-carboxylate 2.103

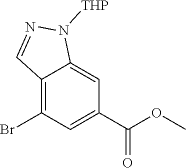

To a mixture of DHP (2.47 g, 29.40 mmol) and Methyl 4-bromo-1H-indazole-6-carboxylate (CAS 885518-47-8, 5 g, 19.60 mmol) in THF (10 mL) was added TsOH·H₂O (1.86 g, 9.80 mmol) under N₂. The mixture was stirred at 60° C. for 12 h. The mixture was filtered and concentrated in vacuo to give a residue, which was purified (PM60) to afford compound 2.103 (5 g, 14.30 mmol, 72.94% yield) as a light-yellow solid.

LCMS (AM3): rt=0.963 min, (257.1 [M-THP+H]⁺), 97.6% purity

### Synthesis of Intermediate 2.105

### methyl4-((2-(4-((tert-butoxycarbonyl)amino)butoxy)ethyl)amino)-1-(tetrahydro-2H-pyran-2-yl)-1H-indazole-6-carboxylate 2.104

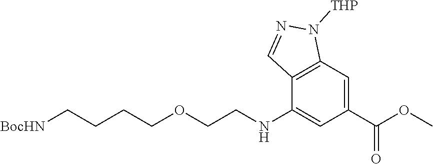

To a mixture of compound 2.103 (2 g, 5.90 mmol) and tert-Butyl (4-(2-aminoethoxy)butyl)carbamate (CAS 2830620-37-4, 1.64 g, 7.08 mmol) in DMF (3 mL) was added K₂CO₃ (1.63 g, 11.79 mmol), (2S,4S)-4-hydroxypyrrolidine-2-carboxylic acid (309.28 mg, 2.36 mmol) and CuI (224.60 mg, 1.18 mmol) under N₂. The mixture was stirred at 110° C. for 12 h. The mixture was concentrated in vacuo to give a residue, which was purified (PM125) to afford compound 2.104 (0.2 g, 395.44 μmol, 6.71% yield) as a brown oil.

LCMS (AM3): rt=0.986 min, (491.3 [M+H]⁺), 97.4% purity

### methyl 4-((2-(4-aminobutoxy)ethyl)amino)-1H-indazole-6-carboxylate 2.105

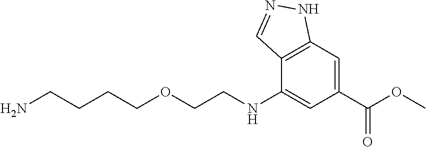 [SMILES: COC(=O)c1cc(NCCOCCCCN)c2cn[nH]c2c1]

To a mixture of compound 2.104 (390 mg, 794.96 μmol) in DCM (10 mL) was added TFA (3.08 g, 27.01 mmol) in one portion. The mixture was stirred at 25° C. for 0.5 h. The mixture was concentrated in vacuo to afford compound 2.105 (300 mg, crude, TFA salt) as a brown oil, which was used for the next step without further purification.

LCMS (AM3): rt=0.679 min, (307.1 [M+H]⁺), 86.6% purity

### Synthesis of Intermediate 2.108

### 4-((2-(4-(((5-(trifluoromethyl)-1H-indol-2-yl)methyl)amino)butoxy)ethyl)amino)-1H-indazole-6-carboxamide 2.107

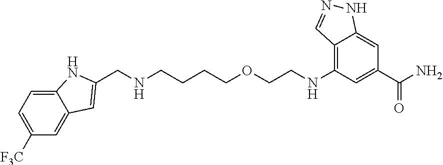 [SMILES: NC(=O)c1cc(NCCOCCCCNCc2cc3cc(C(F)(F)F)ccc3[nH]2)c2cn[nH]c2c1]

To a mixture of EXAMPLE 84 (60 mg, 119.16 μmol) in MeOH (10 mL) was added NH₃ (81.17 mg, 4.77 mmol) at −60° C. The mixture was stirred at 85° C. for 12 h in a sealed tube. The mixture was filtered and the filtrate was concentrated in vacuo to give a residue, which was purified (PM92) to afford compound 2.107 (40 mg, 70.42 μmol, 59.10% yield) as a brown solid.

LCMS (AM3): rt=0.806 min, (489.2 [M+H]⁺), 86.4% purity

### N-(6-cyano-1H-indazol-4-yl)-2,2,2-trifluoro-N-(2-(4-(2,2,2-trifluoro-N-((5-(trifluoromethyl)-1H-indol-2-yl)methyl)acetamido)butoxy)ethyl)acetamide 2.108

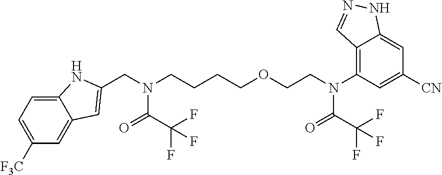 [SMILES: N#Cc1cc(N(CCOCCCCN(Cc2cc3cc(C(F)(F)F)ccc3[nH]2)C(=O)C(F)(F)F)C(=O)C(F)(F)F)c2cn[nH]c2c1]

To a mixture of compound 2.107 (30 mg, 52.81 μmol) in DCM (4 mL) was added TEA (16.03 mg, 158.44 μmol) and TFAA (22.19 mg, 105.63 μmol) at 0° C. under N₂. The mixture was stirred at 25° C. for 1 h. The mixture was filtered and the filtrate was concentrated in vacuo to afford compound 2.108 (30 mg, crude) as a yellow oil, which was used for the next step without further purification.

LCMS (AM3): rt=1.085 min, (663.2 [M+H]⁺), 79.0% purity

### Synthesis of Intermediate 2.113

### tert-butyl (4-((1-(6-bromo-1-(tetrahydro-2H-pyran-2-yl)-1H-indazol-4-yl)azetidin-3-yl)oxy)butyl)carbamate 2.110

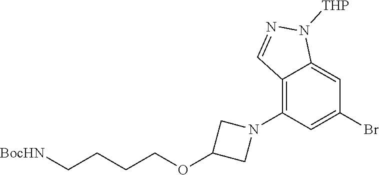

A mixture of tert-butyl (4-(azetidin-3-yloxy)butyl)carbamate (CAS 2830620-55-6, 16.34 g, 66.86 mmol), 6-bromo-4-fluoro-1-(tetrahydro-2H-pyran-2-yl)-1H-indazole (10 g, 33.43 mmol, Organic Letters, 2020, vol. 22, #22, p. 9047-9052) and DIPEA (8.64 g, 66.86 mmol, 11.65 mL) in NMP (250 mL) was stirred at 140° C. for 15 h under N₂. The mixture was cooled to RT, poured into water (500 mL) and extracted with EA (200 mL×2). The combined organic phases were washed with brine, dried (Na₂SO₄), filtered and concentrated in vacuo to give a residue, which was purified (PM42) to afford compound 2.110 (8.5 g, 16.24 mmol, 48.57% yield) as a yellow oil.

LCMS (AM3): rt=1.073 min, (525.4 [M+H]⁺), 42.37% purity

### tert-butyl(4-((1-(1-(tetrahydro-2H-pyran-2-yl)-6-(4,4,5,5-tetramethyl-1,3,2-dioxaborolan-2-yl)-1H-indazol-4-yl)azetidin-3-yl)oxy)butyl carbamate 2.111

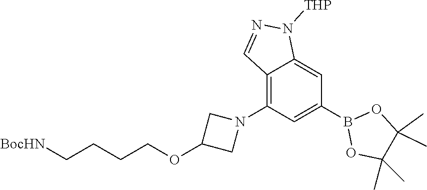

To a mixture of B₂Pin₂ (2.91 g, 11.46 mmol) and compound 2.110 (4 g, 7.64 mmol) in dioxane (50 mL) was added Pd(dppf)Cl₂ (559.13 mg, 764.14 μmol) and KOAc (1.50 g, 15.28 mmol) in one portion under N₂. The mixture was stirred at 85° C. for 12 h. The mixture was filtered and the filtrate was concentrated in vacuo to give a residue, which was purified (PM50) to afford compound 2.111 (3.1 g, 4.46 mmol, 58.31% yield) as a yellow oil.

LCMS (AM3): rt=0.913 min, (571.3 [M+H]⁺), 82.0% purity

### tert-butyl (4-((1-(6-(pyridazin-4-yl)-1-(tetrahydro-2H-pyran-2-yl)-1H-indazol-4-yl)azetidin-3-yl)oxy)butyl)carbamate 2.112

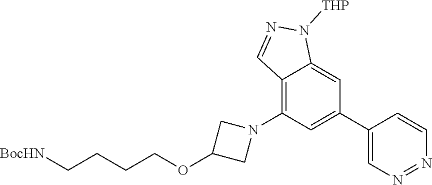

To a mixture of 4-bromopyridazine (1.39 g, 5.79 mmol) and compound 2.111 (3.1 g, 4.46 mmol) in dioxane (30 mL) and H₂O (3 mL) was added K₂CO₃ (1.23 g, 8.91 mmol) and Pd(dppf)Cl₂·CH₂Cl₂ (363.86 mg, 445.55 μmol) in one portion under N₂. The mixture was stirred at 90° C. for 12 h. The mixture was filtered and the filtrate was concentrated in vacuo to give a residue, which was purified (PM125) to afford compound 2.112 (1.8 g, 2.62 mmol, 58.75% yield) as a brown solid.

LCMS (AM3): rt=0.968 min, (523.5 [M+H]⁺), 76.8% purity

### 4-((1-(6-(pyridazin-4-yl)-1H-indazol-4-yl) azetidin-3-yl) oxy) butan-1-amine 2.113

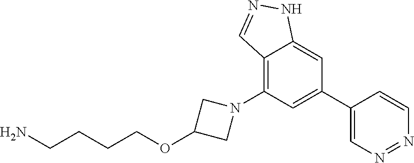 [SMILES: NCCCCOC1CN(c2cc(-c3ccnnc3)cc3[nH]ncc23)C1]

To a mixture of compound 2.112 (1.8 g, 2.62 mmol) in DCM (40 mL) was added TFA (25.82 g, 226.42 mmol) at 0° C. The mixture was stirred at 25° C. for 2 h. The mixture was filtered and the filtrate was concentrated in vacuo to give a residue, which was purified (PM126) to afford compound 2.113 (1.2 g, crude) as a brown gum, which was used without further purification.

LCMS (AM3): rt=0.485 min, (423.1 [M+H]⁺), 62.0% purity

### Synthesis of Intermediate 2.115

### tert-butyl (4-((1-(6-(isoxazol-4-yl)-1-(tetrahydro-2H-pyran-2-yl)-1H-indazol-4-yl)azetidin-3-yl)oxy)butyl)carbamate 2.114

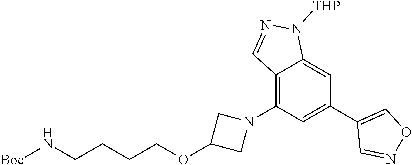

A mixture of compound 2.110 (5 g, 9.55 mmol), 4-(4,4,5,5-tetramethyl-1,3,2-dioxaborolan-2-yl)isoxazole (2.24 g, 11.46 mmol), Pd(dppf)Cl₂ (698.91 mg, 955.18 μmol) and KF (1.66 g, 28.66 mmol) in DMF (95 mL) and H₂O (28 mL) was stirred at 60° C. for 2.5 h under N₂. The mixture was poured into water (100 mL) and extracted with EA (80 mL×3). The combined organic phase was washed (brine, 200 mL×3), dried (Na₂SO₄), filtered and concentrated in vacuo to give a residue, which was purified (PM3) to afford compound 2.114 (2.8 g, 4.98 mmol, 52.14% yield) as a yellow oil.

LCMS (AM3): rt=0.987 min, (512.5 [M+H]⁺), 91% purity.

### 4-((1-(6-(isoxazol-4-yl)-1H-indazol-4-yl)azetidin-3-yl)oxy)butan-1-amine 2.115

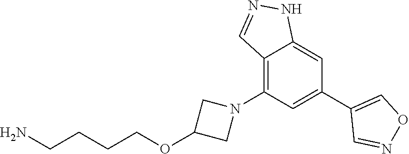 [SMILES: NCCCCOC1CN(c2cc(-c3cnoc3)cc3[nH]ncc23)C1]

A solution of compound 2.114 (2.8 g, 5.47 mmol) in TFA (35.93 g, 315.15 mmol) was stirred at 20° C. for 0.5 h. The mixture was concentrated in vacuo to give a residue, which was purified (PM88) to afford compound 2.115 (800 mg, 2.10 mmol, 38.36% yield, FA salt) as a green solid.

LCMS (AM3): rt=0.717 min, (328.4 [M+H]⁺), 98.4% purity.

### Synthesis of Intermediate 2.117

### tert-butyl (4-(2-((6-(pyrimidin-4-yl)-1-(tetrahydro-2H-pyran-2-yl)-1H-indazol-4-yl)amino)ethoxy)butyl)carbamate 2.116

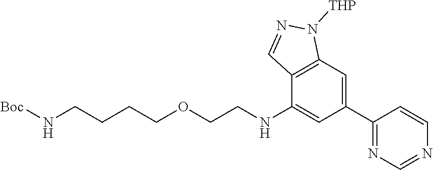

To a mixture of compound 1.919 (300 mg, 537.14 μmol) and 4-chloropyrimidine (CAS 17180-93-7, 89.21 mg, 590.85 μmol, HCl salt) in dioxane (10 mL) and H₂O (1 mL) was added K₂CO₃ (222.71 mg, 1.61 mmol) and Pd(dppf)Cl₂ (39.30 mg, 53.71 μmol). The mixture was stirred at 80° C. for 12 h under N₂. The mixture was concentrated in vacuo to give a residue, which was purified (PM32) to afford compound 2.116 (100 mg, 195.84 μmol, 36.46% yield) as a yellow oil.

LCMS (AM3): rt=0.947 min, (511.5 [M+H]⁺), 74.6% purity

### N-(2-(4-aminobutoxy)ethyl)-6-(pyrimidin-4-yl)-1H-indazol-4-amine 2.117

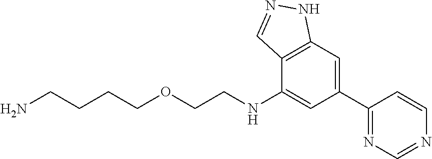 [SMILES: NCCCCOCCNc1cc(-c2ccncn2)cc2[nH]ncc12]

A mixture of compound 2.116 (400 mg, 783.35 μmol) in HCl/MeOH (4 M, 5 mL) was stirred at 20° C. for 1 h. The mixture was concentrated in vacuo to afford compound 2.117 (340 mg, crude, HCl salt) as a red oil, which was used without further purification.

LCMS (AM5): rt=0.797 min, (327.3 [M+H]⁺), 96.79% purity

### Synthesis of Intermediate 2.221

### 4-fluoro-1-(tetrahydro-2H-pyran-2-yl)-1H-indazol-6-amine 2.118

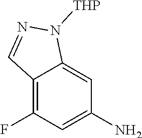

A mixture of 6-bromo-4-fluoro-1-(tetrahydro-2H-pyran-2-yl)-1H-indazole (20 g, 66.86 mmol), CuI (2.55 g, 13.38 mmol), K₂CO₃ (28 g, 202.59 mmol), NH₃·H₂O (324.54 mmol, 50 mL, 25% purity) and (2S,4S)-4-hydroxypyrrolidine-2-carboxylic acid (3.51 g, 26.74 mmol) in DMSO (200 mL) was stirred at 90° C. for 18 h under N₂. Water (600 mL) was added and the mixture was extracted with EA (100 mL×4). The combined organic phases were washed (brine, 200 mL), dried (Na₂SO₄), filtered and concentrated in vacuo to afford compound 2.118 (13.7 g, 55.90 mmol, 83.62% yield) as a brown solid, which was used without further purification.

LCMS (AM3): rt=0.769 min, (236.2 [M+H]⁺), 96.2% purity.

### 4-fluoro-1-(tetrahydro-2H-pyran-2-yl)-6-(4H-1,2,4-triazol-4-yl)-1H-indazole 2.119

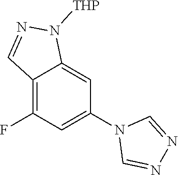

To a mixture of compound 2.118 (13.7 g, 58.23 mmol), N-formamidoformamide (25.65 g, 291.22 mmol) and TEA (409.52 mmol, 57 mL) in pyridine (200 mL) was added TMSCl (866.72 mmol, 110 mL) under N₂. The reaction mixture was stirred at 120° C. for 14 h under N₂. The reaction mixture was concentrated in vacuo to give a residue. Water (200 mL) and EA (200 mL) were added and a solid precipitated. The solid was filtered and the filter cake was dried under vacuum to afford compound 2.119 (10 g, 34.81 mmol, 59.77% yield) as a white solid.

LCMS (AM3): rt=0.818 min, (288.1 [M+H]⁺), 92.3% purity.

### tert-butyl (4-((1-(1-(tetrahydro-2H-pyran-2-yl)-6-(4H-1,2,4-triazol-4-yl)-1H-indazol-4-yl)azetidin-3-yl)oxy)butyl)carbamate 2.220

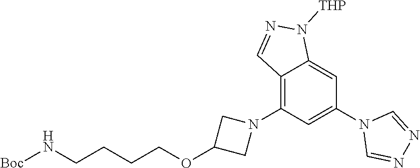

A mixture of tert-butyl (4-(azetidin-3-yloxy)butyl)carbamate (Ref: WO2022185041, 5.10 g, 20.88 mmol), compound 2.119 (3 g, 10.44 mmol) and DIPEA (2.70 g, 20.88 mmol) in NMP (30 mL) was stirred at 140° C. for 15 h under N₂. The reaction mixture was diluted with H₂O (60 mL) and extracted with EA (80 mL×2). The organic layers were washed (brine, 100 mL), dried (Na₂SO₄), filtered and concentrated in vacuo to give a residue, which was purified (PM127) to afford compound 2.220 (4 g, 7.82 mmol, 74.87% yield) as a yellow oil.

LCMS (AM3): rt=0.812 min, (512.2 [M+H]⁺), 100% purity.

### 4-((1-(6-(4H-1,2,4-triazol-4-yl)-1H-indazol-4-yl)azetidin-3-yl)oxy)butan-1-amine 2.221

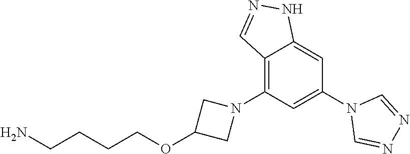 [SMILES: NCCCCOC1CN(c2cc(-n3cnnc3)cc3[nH]ncc23)C1]

To a solution of compound 2.220 (4 g, 7.82 mmol) in DCM (1 mL) was added TFA (30.80 g, 270.12 mmol). The resulting mixture was stirred at 25° C. for 1 h. The reaction mixture was concentrated in vacuo to give a residue, which was purified (PM63) to afford compound 2.221 (2.8 g, 7.50 mmol, 95.91% yield, FA salt) as a yellow solid.

LCMS (AM5): rt=0.741 min, (328.1 [M+H]⁺), 81.2% purity.

### Synthesis of Intermediate 2.225

### tert-butyl methyl(2-oxo-2-((1-(tetrahydro-2H-pyran-2-yl)-6-(4H-1,2,4-triazol-4-yl)-1H-indazol-4-yl)amino)ethyl)carbamate 2.222

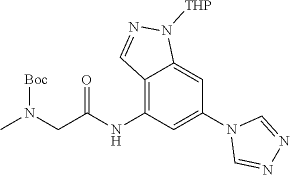

To a solution of N-(tert-butoxycarbonyl)-N-methylglycine (732.04 mg, 3.87 mmol) in pyridine (4 mL) was added EDCI (2.02 g, 10.55 mmol) and compound 1.273 (1 g, 3.52 mmol). The mixture was stirred at 80° C. for 12 h. The mixture was concentrated in vacuo to give a residue, which was purified (PM128) and lyophilized to afford compound 2.222 (459 mg, 1.01 mmol, 28.65% yield) as a light-yellow solid.

LCMS (AM3): rt=0.849 min, (456.2 [M+H]⁺), 19.5% purity

### tert-butyl methyl(2-((1-(tetrahydro-2H-pyran-2-yl)-6-(4H-1,2,4-triazol-4-yl)-1H-indazol-4-yl)amino)ethyl)carbamate 2.223

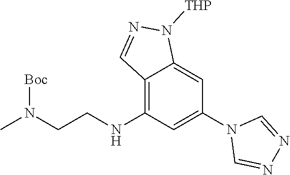

A solution of compound 2.222 (459 mg, 1.01 mmol) in THF (10 mL) was added BH₃.THF (1 M, 10.08 mL) at 0° C., then the mixture was stirred at 20° C. for 3 h. The mixture was quenched with methanol (5 mL) and concentrated in vacuo to give the residue, which was purified (PM129) to afford compound 2.223 (210 mg, 475.62 μmol, 47.20% yield) as a red solid.

LCMS (AM3): rt=0.835 min, (442.2 [M+H]⁺), 66.4% purity

### N1-(6-(4H-1,2,4-triazol-4-yl)-1H-indazol-4-yl)-N2-methylethane-1,2-diamine 2.224

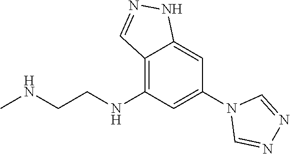 [SMILES: CNCCNc1cc(-n2cnnc2)cc2[nH]ncc12]

A mixture of compound 2.223 (210 mg, 475.62 μmol) in HCl/MeOH (4 M, 4 mL) was stirred at 25° C. for 0.5 h. The mixture was concentrated in vacuo to afford compound 2.224 (190 mg, crude, HCl salt) as a red solid, which was used directly for the next step.

LCMS (AM3): rt=0.120 min, (258.6 [M+H]⁺), 68.6% purity

### tert-butyl (4-((2-((6-(4H-1,2,4-triazol-4-yl)-1H-indazol-4-yl)amino)ethyl)(methyl)amino)butyl)(3-chloro-4-(trifluoromethoxy)benzyl)carbamate 2.225

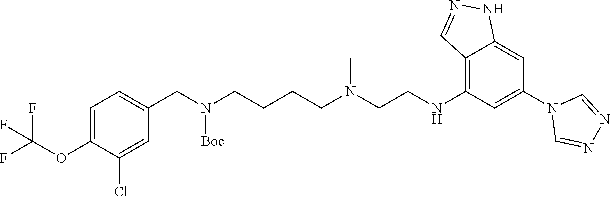 [SMILES: CN(CCCCN(Cc1ccc(OC(F)(F)F)c(Cl)c1)C(=O)OC(C)(C)C)CCNc1cc(-n2cnnc2)cc2[nH]ncc12]

A solution of compound 2.224 (100 mg, 340.42 μmol, HCl salt) and Intermediate I (134.74 mg, 340.42 μmol) in MeOH (3 mL) was added NaBH₃CN (64.18 mg, 1.02 mmol) and DIPEA (87.99 mg, 680.84 μmol). The mixture was stirred at 25° C. for 2 h. The mixture was concentrated in vacuo to give a residue, which was purified (PM156) to afford compound 2.225 (90 mg, 141.27 μmol, 41.50% yield) as a red solid.

LCMS (AM3): rt=0.864 min, (637.2 [M+H]⁺), 71% purity

### Synthesis of Intermediate 2.231

### methyl 4-nitro-1-(tetrahydro-2H-pyran-2-yl)-1H-indazole-6-carboxylate 2.228

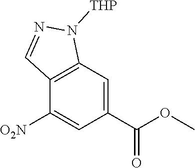

To a solution of methyl 4-nitro-1H-indazole-6-carboxylate (CAS 72922-61-3, 1.8 g, 8.41 mmol) in THF (8 mL) was added 3,4-dihydro-2H-pyran (2.74 g, 32.55 mmol) followed by TsOH (280.30 mg, 1.63 mmol). The mixture was heated to 70° C. and stirred for 12 h. The mixture was concentrated in vacuo to give a residue, which was purified (PM42) to afford compound 2.228 (1.7 g, 5.57 mmol, 68.42% yield) as a yellow oil.

¹H NMR (400 MHz, CHCl₃-d) 5 8.77-8.78 (m, 1H), 8.69 (d, J=6.0 Hz, 2H), 5.88 (dd, J=9.2 Hz, 2.8 Hz, 1H), 4.04 (s, 3H), 3.89-3.73 (m, 2H), 2.62-2.45 (m, 2H), 1.82-1.72 (m, 4H) ppm.

### 4-nitro-1-(tetrahydro-2H-pyran-2-yl)-1H-indazole-6-carbohydrazide 2.229

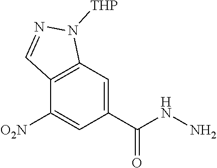

To a solution of methyl compound 2.228 (1.7 g, 5.57 mmol) in EtOH (25 mL) was added N₂H₄·H₂O (1.14 g, 22.27 mmol, 98% purity) at 20° C. The mixture was heated to 70° C. and stirred for 2 h. The mixture was cooled to 20° C. and filtered. The filter cake was washed with EtOH (10 mL×3), filtered and dried under vacuum to afford compound 2.229 (1.1 g, 3.60 mmol, 64.71% yield) as a yellow solid.

### 2-(4-nitro-1-(tetrahydro-2H-pyran-2-yl)-1H-indazol-6-yl)-1,3,4-oxadiazole 2.230

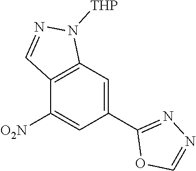

To a solution of compound 2.229 (1.1 g, 3.60 mmol) in dioxane (30 mL) was added trimethoxymethane (3.87 g, 36.49 mmol) and TsOH (62.05 mg, 360.31 μmol). The mixture was heated to 90° C. and stirred for 4 h. The mixture was concentrated in vacuo to give a residue, which was purified (PM38) to afford compound 2.230 (940 mg, 2.94 mmol, 81.50% yield) as a white solid.

LCMS (AM12): rt=0.530 min, (316.1 [M+H]⁺), 98.51% purity.

### 6-(1,3,4-oxadiazol-2-yl)-1-(tetrahydro-2H-pyran-2-yl)-1H-indazol-4-amine 2.231

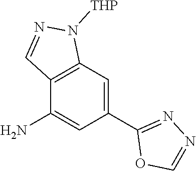

To a mixture of compound 2.230 (940 mg, 2.98 mmol) in MeOH (15 mL) and H₂O (5 mL) was added NH₄Cl (1.28 g, 23.85 mmol) and Fe (998.99 mg, 17.89 mmol) at 20° C. The mixture was heated to 50° C. and stirred for 2 h. The mixture was poured onto sat. NaHCO₃ (aq.) (50 mL) and extracted with EA (60 mL×2). The combined organic phase was washed (brine, 40 mL×2), dried (Na₂SO₄), filtered and concentrated in vacuo to afford compound 2.231 (800 mg, 2.75 mmol, 92.17% yield) as a yellow solid.

LCMS (AM12): rt=0.388 min, (286.0 [M+H]⁺), 98.01% purity.

### Synthesis of Intermediate 2.235

### 4-nitro-1-(tetrahydro-2H-pyran-2-yl)-6-(1H-1,2,3-triazol-1-yl)-1H-indazole 2.234

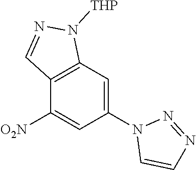

To a mixture of compound 1.271 (3 g, 11.44 mmol) in ACN (30 mL) was added isopentyl nitrite (13.40 g, 114.39 mmol) followed by TMSN₃ (13.18 g, 114.39 mmol) in portions at 0° C. The mixture was stirred at 25° C. for 16 h. To the above mixture was added vinyl acetate (4.93 g, 57.24 mmol). The mixture was heated to 80° C. and stirred for 48 h. The mixture was poured into water (150 mL) and extracted with EA (150 mL×3). The combined organic phase was washed (brine, 150 mL), dried with anhydrous sodium sulfate, filtered and concentrated in vacuo to give a residue, which was purified (PM27) to afford compound 2.234 (1.9 g, 6.05 mmol, 52.81% yield) as a brown solid.

LCMS (AM12): rt=0.666 min, (315.2 [M+H]⁺), 59.9% purity.

¹H NMR (400 MHz, CHCl₃-d) 5 8.72 (s, 1H), 8.55 (s, 1H), 8.49 (d, J=1.6 Hz, 1H), 8.20 (d, J=1.2 Hz, 1H), 7.96 (d, J=1.2 Hz, 1H), 5.90 (dd, J=8.8 Hz, 2.8 Hz, 1H), 4.03-3.97 (m, 1H), 3.84-3.78 (m, 1H), 2.61-2.54 (m, 1H), 2.26-2.17 (m, 2H), 1.84-1.74 (m, 3H) ppm.

### 1-(tetrahydro-2H-pyran-2-yl)-6-(1H-1,2,3-triazol-1-yl)-1H-indazol-4-amine 2.235

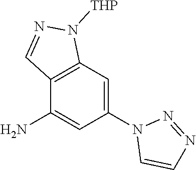

To a mixture of compound 2.234 (1.57 g, 5.00 mmol) in MeOH (20 mL) and H₂O (4 mL) was added Fe (1.39 g, 24.98 mmol) and NH₄Cl (1.87 g, 34.97 mmol). The mixture was heated to 50° C. and stirred for 6 h. The mixture was filtered and the filter cake was washed with MeOH (20 mL). The filtrate was concentrated in vacuo to afford compound 2.235 (920 mg, 3.24 mmol, 64.78% yield) as a brown solid.

LCMS (AM12): rt=0.408 min, (285.1 [M+H]⁺), 99.5% purity.

### Synthesis of Intermediate 2.238

### 6-(3-fluoropyridin-4-yl)-4-nitro-1-(tetrahydro-2H-pyran-2-yl)-1H-indazole 2.237

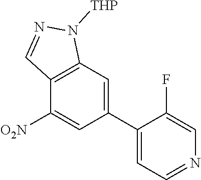

To a solution of compound 1.943 (1 g, 2.68 mmol) in dioxane (10 mL) and H₂O (1 mL) was added 4-bromo-3-fluoro-pyridine (565.86 mg, 3.22 mmol) followed by addition of Pd(dppf)Cl₂ (196.06 mg, 267.95 μmol) and Cs₂CO₃ (1.75 g, 5.36 mmol). The reaction mixture was stirred at 100° C. for 16 h. The residue was diluted with water (50 mL) and extracted with EA (40 mL×4). The combined organic layers were washed with saturated brine (50 mL×3), dried (Na₂SO₄), filtered and concentrated in vacuo to give a residue, which was purified (PM27) to afford compound 2.237 (937 mg, 2.68 mmol, 95.25% yield) as a grey solid.

LCMS (AM7): rt=0.931 min, (343.0 [M+H]⁺), 97.93% purity.

### 6-(3-fluoropyridin-4-yl)-1-(tetrahydro-2H-pyran-2-yl)-1H-indazol-4-amine 2.238

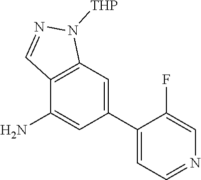

To a solution of compound 2.237 (750 mg, 2.19 mmol) in a mixture solvent of EtOH (7.5 mL) and H₂O (1.5 mL) was added NH₄Cl (585.96 mg, 10.95 mmol) and Fe (611.76 mg, 10.95 mmol). The reaction mixture was stirred at 80° C. for 1 h. The reaction mixture was filtered. The filtrate was concentrated in vacuo to remove the organic solvent. The residue was diluted with water (35 mL) and extracted with EA (30 mL×4). The combined organic layers were washed (brine, 30 mL×2), dried (Na₂SO₄), filtered and concentrated in vacuo to give a residue, which was purified (PM38) to afford compound 2.238 (657 mg, 2.04 mmol, 87.13% yield) as a red oil.

LCMS (AM7): rt=0.743 min, (313.1 [M+H]⁺), 97.11% purity.

### Synthesis of Intermediate 2.241

### tert-butyl 3-chloro-4-(trifluoromethoxy)benzyl(3-oxo-3-((3-((6-(6-oxo-1,6-dihydropyridazin-4-yl)-1-(tetrahydro-2H-pyran-2-yl)-1H-indazol-4-yl)amino)propyl)amino)propyl)carbamate 2.241

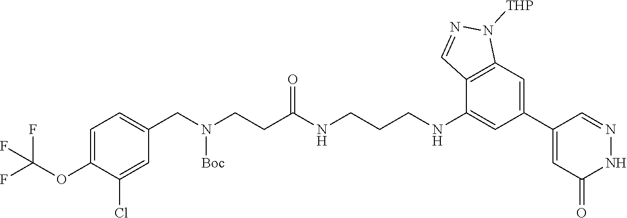

To a solution of compound 1.950 (152.73 mg, 337.25 μmol) and compound 1.953 (70 mg, 224.84 μmol) in MeOH (1 mL) was added MgSO₄ (135.32 mg, 1.12 mmol). The mixture was stirred for 12 h, then NaBH₃CN (49.45 mg, 786.93 μmol) was added and the mixture was stirred at 20° C. for 2 h. The mixture was concentrated in vacuo to give a residue, which was purified (PM135) to afford compound 2.241 (55 mg, 57.57 μmol, 25.60% yield) as a white solid.

LCMS (AM16): rt=0.756 min, (748.4 [M+H]⁺), 78.31% purity.

The following Intermediates in Table 4 were made with non-critical changes or substitutions to the exemplified procedure for Intermediate 1.950 that would be understood by one skilled in the art, wherein R may or may not contain a THP protecting group.

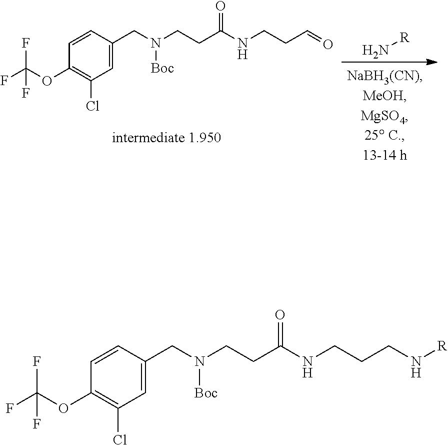

TABLE 4

| H₂N—R | | |
|---|---|---|
| Reactant IUPAC name | Product IUPAC name | |
| (Intermediate No.) | (Intermediate No.) | Analytical |
| 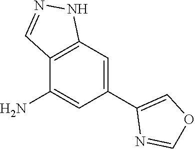 | tert-butyl (3-chloro-4- (trifluoromethoxy) benzyl)(3-((3-((6- (oxazol-4-yl)-1H- indazol-4-yl)amino) propyl)amino)-3- oxopropyl)carbamate 2.341 | LCMS (AM14): rt = 0.651 min, (637.3 [M + H]⁺), 99.20% purity. Purification Method PM178 |
| 6-(oxazol-4-yl)-1H- | | |
| indazol-4-amine 2.340 | | |
| 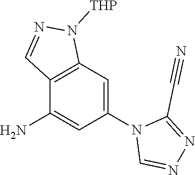 | tert-butyl (3-chloro-4- (trifluoromethoxy) benzyl)(3-((3-((6-(3-cyano- 4H-1,2,4-triazol-4-yl)- 1-(tetrahydro-2H- pyran-2-yl)-1H- yl)amino)propyl)amino)- 3-oxopropyl)carbamate 2.459 | LCMS (AM11): rt = 0.564 min, (746.2, [M + H]⁺), 95.36% purity. Purification Method PM198 |
| 4-(4-amino-1-(tetrahydro- | | |
| 2H-pyran-2-yl)-1H-indazol-6- | | |
| yl)-4H-1,2,4-triazole-3- | | |
| carbonitrile 2.457 | | |
| 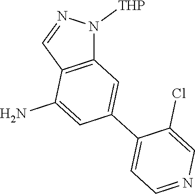 | tert-butyl (3-chloro-4- (trifluoromethoxy)benzyl) (3-((3-((6-(3- chloropyridin-4-yl)-1- (tetrahydro-2H-pyran- 2-yl)-1H-indazol-4- yl)amino)propyl)amino)- 3-oxopropyl)carbamate 2.481 | LCMS (AM12): rt = 0.688 min, (765.6, [M + H]⁺), 89.15% purity. Purification Method PM202 |
| 6-(3-chloropyridin-4-yl)- | | |
| 1-(tetrahydro-2H-pyran- | | |
| 2-yl)-1H-indazol-4- | | |
| amine 2.479 | | |
| 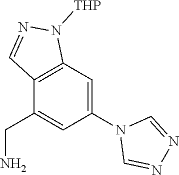 | tert-butyl (3-chloro-4- (trifluoromethoxy)benzyl) (3-oxo-3-((3-(((1- (tetrahydro-2H-pyran- 2-yl)-6-(4H-1,2,4- triazol-4-yl)-1H- indazol-4- yl)methyl)amino)propyl) amino)propyl) carbamate 2.489 | LCMS (AM14): rt = 0.903 min, (735.4, [M + H]⁺), 99.46% purity Purification Method PM203 |
| (1-(tetrahydro-2H- | | |
| pyran-2-yl)-6-(4H-1,2,4- | | |
| triazol-4-yl)-1H-indazol- | | |
| 4-yl)methanamine 2.488 | | |

### Synthesis of Intermediate 2.243

### 6-(2-chloropyrimidin-4-yl)-4-nitro-1-(tetrahydro-2H-pyran-2-yl)-1H-indazole 2.242

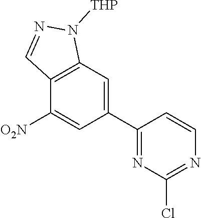

To a solution of compound 1.943 (2 g, 5.36 mmol) in dioxane (10 mL) and H₂O (1 mL) was added 2,4-dichloropyrimidine (725.78 mg, 4.87 mmol), K₂CO₃ (2.02 g, 14.62 mmol) and Pd(dppf)Cl₂ (356.47 mg, 487.17 μmol). The mixture was stirred at 80° C. for 16 h under N₂. The mixture was filtered and the filter cake was washed with EA (10 mL). The filtrate was concentrated in vacuo to give a residue, which was purified (PM35) to afford compound 2.242 (1.59 g, 4.25 mmol, 87.20% yield) as a white solid.

LCMS (AM11): rt=0.464 min, (360.1 [M+H]⁺), 96.77% purity.

¹H NMR (400 MHz, CHCl₃-d) δ 8.83 (s, 1H), 8.81-8.74 (m, 2H), 8.71 (s, 1H), 7.84 (d, J=5.2 Hz, 1H), 5.98 (dd, J=8.4 Hz, 2.4 Hz, 1H), 4.04-3.94 (m, 1H), 3.91-3.77 (m, 1H), 2.67-2.53 (m, 1H), 2.26-2.15 (m, 2H), 1.89-1.73 (m, 3H) ppm.

### 6-(2-chloropyrimidin-4-yl)-1-(tetrahydro-2H-pyran-2-yl)-1H-indazol-4-amine 2.243

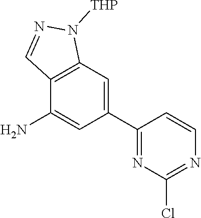

To a solution of compound 2.242 (500 mg, 1.39 mmol) in MeOH (10 mL) and H₂O (2 mL) was added Fe (232.84 mg, 4.17 mmol) and NH₄Cl (371.71 mg, 6.95 mmol). The mixture was stirred at 50° C. for 5 h. The mixture was filtered and washed with MeOH (10 mL). The filtrate was concentrated in vacuo to afford compound 2.243 (371 mg, 933.73 μmol, 67.19% yield) as a yellow solid.

LCMS (AM16): rt=0.608 min, (330.1 [M+H]⁺), 83.56% purity.

### Synthesis of Intermediate 2.247

### tert-butyl (3-chloro-4-(trifluoromethoxy)benzyl)(3-((3-((6-(2-chloropyrimidin-4-yl)-1-(tetrahydro-2H-pyran-2-yl)-1H-indazol-4-yl)amino)propyl)amino)-3-oxopropyl)carbamate 2.246

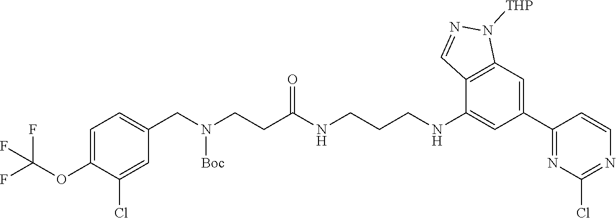

To a solution of compound 2.243 (100 mg, 303.23 μmol) in MeOH (2 mL) was added compound 1.950 (137.32 mg, 303.23 μmol) and MgSO₄ (182.50 mg, 1.52 mmol). The mixture was stirred at 20° C. for 12 h, then NaBH₃CN (66.69 mg, 1.06 mmol) was added. The mixture was stirred at 20° C. for 0.5 h. The reaction mixture was poured into water (10 mL) and extracted with EA (10 mL×3). The combined organic phase was washed (brine, 10 mL), dried (Na₂SO₄), filtered and concentrated in vacuo to give a residue, which was purified (PM23) to afford compound 2.246 (62 mg, 77.94 μmol, 25.70% yield) as a yellow oil.

LCMS (AM16): rt=0.867 min, (766.4 [M+H]⁺), 96.37% purity.

### tert-butyl (3-chloro-4-(trifluoromethoxy)benzyl)(3-oxo-3-((3-((6-(2-oxo-1,2-dihydropyrimidin-4-yl)-1-(tetrahydro-2H-pyran-2-yl)-1H-indazol-4-yl)amino)propyl)amino)propyl)carbamate 2.247

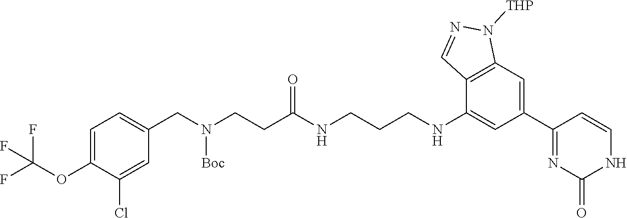

To a solution of compound 2.246 (62 mg, 80.87 μmol) in dioxane (1 mL) and H₂O (1 mL) was added DABCO (9.07 mg, 80.87 μmol) and K₂CO₃ (22.35 mg, 161.75 μmol). The mixture was stirred at 70° C. for 12 h. The reaction mixture was poured into water (10 mL) and extracted with EA (10 mL×3). The combined organic phase was washed (brine, 10 mL), dried (Na₂SO₄), filtered and concentrated in vacuo to afford compound 2.247 (50.15 mg, 54.11 μmol, 66.91% yield) as a yellow solid.

LCMS (AM16): rt=0.654 min, (748.4 [M+H]⁺), 80.73% purity.

### Synthesis of Intermediate 2.248

### tert-butyl(4-(2-((6-cyano-1-(tetrahydro-2H-pyran-2-yl)-1H-indazol-4-yl)amino)ethoxy)butyl)(3,5-difluoro-4-(trifluoromethoxy)benzyl)carbamate 2.248

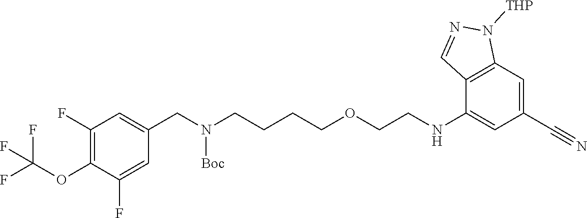

To a mixture of compound 1.941 (100 mg, 226.56 μmol) and compound 1.87 (54.89 mg, 226.56 μmol) in MeOH (2 mL) was added MgSO₄ (136.35 mg, 1.13 mmol) and AcOH (13.60 mg, 226.56 μmol). The mixture was stirred at 20° C. for 12 h, then to the mixture was added NaBH₃CN (49.83 mg, 792.95 μmol). The resulting mixture was stirred at 20° C. for 1 h. The mixture was poured into water (20 mL) and extracted with EA (20 mL×3). The combined organic phase was washed (brine, 20 mL), dried with anhydrous sodium sulfate, filtered and concentrated in vacuo to give a residue, which was purified (PM157) to afford compound 2.248 (71 mg, 105.49 μmol, 46.56% yield) as a yellow oil.

LCMS (AM12): rt=0.735 min, (668.5 [M+H]⁺), 99.20% purity.

### Synthesis of Intermediate 2.251

### 6-(5-methyl-1-(tetrahydro-2H-pyran-2-yl)-1H-pyrazol-4-yl)-1-(tetrahydro-2H-pyran-2-yl)-1H-indazol-4-amine 2.251

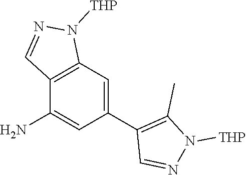

To a solution of compound 1.952 (2 g, 5.83 mmol) in H₂O (2 mL) and dioxane (20 mL) was added 4-Bromo-5-methyl-1-(tetrahydro-2H-pyran-2-yl)-1H-pyrazole (Ref: WO2021127166, 1.71 g, 6.99 mmol) followed by Pd(dppf)Cl₂ (426.37 mg, 582.70 μmol) and Cs₂CO₃ (3.80 g, 11.65 mmol). The reaction mixture was stirred at 100° C. for 16 h. The residue was diluted with water (60 mL) and extracted with EA (40 mL×3). The combined organic layers were washed (brine, 60 mL×2), dried (Na₂SO₄), filtered and concentrated in vacuo to give a residue, which was purified (PM34) to afford compound 2.251 (1 g, 2.54 mmol, 43.64% yield) as a brown solid.

LCMS (AM7): rt=0.787 min, (382.2 [M+H]⁺), 97.0% purity.

### Synthesis of Intermediate 2.253

### tert-butyl(3-chloro-4-(trifluoromethoxy)benzyl)(3-((3-((6-cyano-1-(tetrahydro-2H-pyran-2-yl)-1H-indazol-4-yl)amino)propyl)amino)-3-oxopropyl)carbamate 2.253

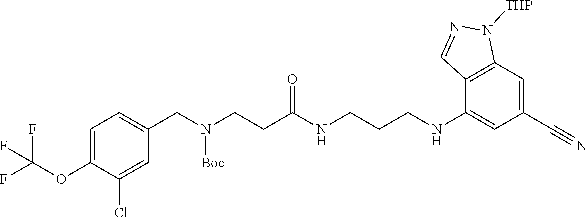

To a mixture of compound 1.950 (120 mg, 264.99 μmol) in MeOH (2 mL) was added compound 1.87 (64.20 mg, 264.99 μmol). The mixture was stirred at 25° C. for 16 h, then to the mixture was added NaBH₃CN (49.96 mg, 794.96 μmol) in one portion, the mixture was stirred at 25° C. for 1 h. The mixture was poured into water (10 mL) and extracted with EA (10 mL×3). The combined organic phase was washed (brine, 10 mL×3), dried (Na₂SO₄), filtered and concentrated in vacuo to give a residue, which was purified (PM24) to afford compound 2.253 (63 mg, 92.77 μmol, 35.01% yield) as a yellow oil.

LCMS (AM11): rt=0.545 min, (679.2 [M+H]⁺), 37.2% purity.

¹H NMR (400 MHz, CHCl₃-d) δ 8.15 (s, 1H), 7.35-7.32 (m, 1H), 7.30-7.28 (m, 1H), 7.26 (s, 1H), 7.17-7.08 (m, 1H), 6.34 (s, 1H), 5.65 (dd, J=9.6 Hz, 2.4 Hz, 1H), 5.60-5.47 (m, 1H), 4.41-4.35 (m, 2H), 4.06-4.00 (m, 1H), 3.78-3.72 (m, 1H), 3.52-3.56 (m, 2H), 3.42-3.30 (m, 4H), 2.53-2.47 (m, 2H), 2.20-2.05 (m, 2H), 2.02-1.97 (m, 1H), 1.87-1.82 (m, 2H), 1.80-1.66 (m, 2H), 1.48-1.45 (m, 9H) ppm.

### Synthesis of Intermediate 2.255

### tert-butyl(4-(2-((7-chloro-6-(pyridin-4-yl)-1-(tetrahydro-2H-pyran-2-yl)-1H-indazol-4-yl)amino)ethoxy)butyl)(3,5-difluoro-4-(trifluoromethoxy)benzyl)carbamate 2.255

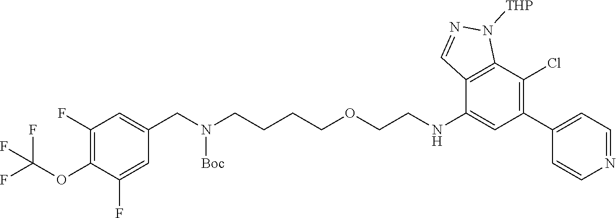

To a solution of compound 2.024 (55 mg, 167.28 μmol) in MeOH (1.5 mL) was added compound 1.941 (73.83 mg, 167.28 μmol), AcOH (12.05 mg, 200.73 μmol) and MgSO₄ (100.67 mg, 836.39 μmol). The mixture was stirred at 20° C. for 12 h, then NaBH₃CN (36.79 mg, 585.47 μmol) was added in portions at 20° C. The resulting mixture was stirred at 20° C. for 1 h. The reaction mixture was filtered and the filtrate was concentrated in vacuo to give a residue, which was purified (PM160) to afford compound 2.255 (48 mg, 62.80 μmol, 37.55% yield) as a light-yellow oil.

LCMS (AM16): rt=0.987 min, (754.4 [M+H]⁺), 98.68% purity.

The following Intermediates in Table 5 were made with non-critical changes or substitutions to the exemplified procedure for Intermediate 2.255 that would be understood by one skilled in the art, wherein R may or may not contain a THP protecting group.

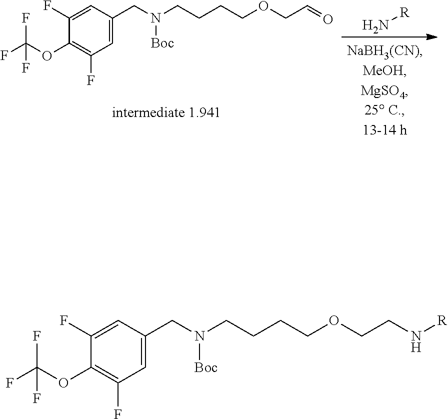

TABLE 5

| NH₂—R | | |
|---|---|---|
| Reactant IUPAC name | Product IUPAC name | |
| (Intermediate No.) | (Intermediate No.) | Analytical |
| 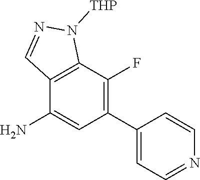 | tert-butyl(3,5-difluoro-4- (trifluoromethoxy)benzyl)(4- (2-((7-fluoro-6-(pyridin-4- yl)-1-(tetrahydro-2H-pyran- 2-yl)-1H-indazol-4- yl)amino)ethoxy)butyl)carba- mate 2.264 | LCMS (AM1): rt = 0.514 min, (738.2 [M + H]⁺), 96.3% purity. Purification Method PM70 |
| 7-fluoro-6-(pyridin-4-yl)-1- | | |
| (tetrahydro-2H-pyran-2-yl)- | | |
| 1H-indazol-4-amine 2.263 | | |
| 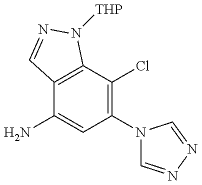 | tert-butyl (4-(2-((7-chloro-1- (tetrahydro-2H-pyran-2-yl)- 6-(4H-1,2,4-triazol-4-yl)- 1H-indazol-4- yl)amino)ethoxy)butyl)(3,5- difluoro-4- (trifluoromethoxy)benzyl)car- bamate 2.376 | LCMS (AM14): rt = 0.765 min, (744.3 [M + H]⁺), 99.25% purity. Purification Method PM195 |
| 7-chloro-1-(tetrahydro-2H- | | |
| pyran-2-yl)-6-(4H-1,2,4- | | |
| triazol-4-yl)-1H-indazol-4- | | |
| amine 2.321 | | |
| 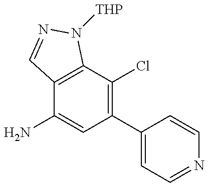 | tert-butyl (3,5-difluoro-4- (trifluoromethoxy)benzyl)(4- (2-((7-methyl-6-(pyridin-4- yl)-1-(tetrahydro-2H- pyran-2-yl)-1H-indazol-4- yl)amino)ethoxy)butyl)carba- mate 2.382 | LCMS (AM7): rt = 1.238 min, (734.3[M + H]⁺), 96.68% purity. Purification Method PM190 |
| 7-methyl-6-(pyridin-4-yl)-1- | | |
| (tetrahydro-2H-pyran-2-yl)-11 | | |
| indazol-4-amine 2.025 | | |
| 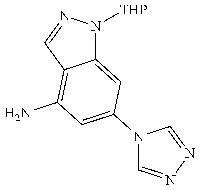 | tert-butyl 3,5-difluoro-4- (trifluoromethoxy)benzyl(4- (2-((1-(tetrahydro-2H- pyran-2-yl)-6-(4H-1,2,4- triazol-4-yl)-1H-indazol-4- yl)amino)ethoxy)butyl)car- bamate 2.395 | LCMS (AM14): rt = 0.729 min, (710.4, [M + H]⁺), 99.1% purity. Purification Method PM172 |
| 1-(Tetrahydro-2H-pyran-2- | | |
| yl)-6-(4H-1,2,4-triazol-4- | | |
| yl)-1H-indazol-4-amine | | |
| 1.273 | | |
| 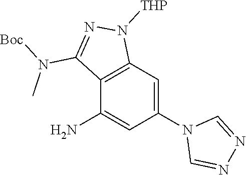 | tert-butyl (4-((2-(4-((tert- butoxycarbonyl)(3,5- difluoro-4- (trifluoromethoxy)benzyl)a- mino)butoxy)ethyl)amino)- 1-(tetrahydro-2H-pyran-2- yl)-6-(4H-1,2,4-triazol-4-yl)- 1H-indazol-3- yl)(methyl)carbamate 2.499 | LCMS (AM11): rt = 0.633 min, (839.5, [M + H]⁺), 84.45% purity. Purification Method PM178 |
| tert-butyl (4-amino-1- | | |
| (tetrahydro-2H-pyran-2-yl)- | | |
| 6-(4H-1,2,4-triazol-4-yl)- | | |
| 1H-indazol-3- | | |
| yl)(methyl)carbamate 2.498 | | |
| 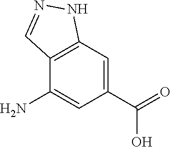 | 4-((2-(4-((tert- butoxycarbonyl)(3,5- difluoro-4- (trifluoromethoxy)benzyl)a- mino)butoxy)ethyl)amino)- 1H-indazole-6-carboxylic acid 2.512 | LCMS (AM11): rt = 0.573 min, (603.3, [M + H]⁺), 99.76% purity. Purification Method PM180 |
| 4-amino-1H-indazole-6- | | |
| carboxylic acid 2.511 | | |
| 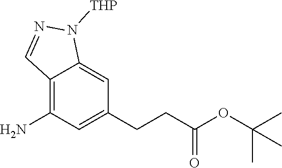 | tert-butyl 3-(4-((2-(4-((tert- butoxycarbonyl)(3,5- difluoro-4- (trifluoromethoxy)benzyl)a- mino)butoxy)ethyl)amino)- 1-(tetrahydro-2H-pyran-2- yl)-1H-indazol-6- yl)propanoate 2.515 | LCMS (AM11): rt = 0.674 min, (771.8, [M + H]⁺), 91.89% purity. Purification Method PM181 |
| tert-butyl 3-(4-amino-1- | | |
| (tetrahydro-2H-pyran-2-yl)- | | |
| 1H-indazol-6- | | |
| yl)propanoate 2.514 | | |

### Synthesis of Intermediate 2.263

### 6-bromo-4-chloro-2,3-difluorobenzaldehyde 2.259

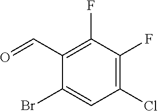 [SMILES: O=Cc1c(Br)cc(Cl)c(F)c1F]

To a solution of LDA (2 M, 5.28 mL) in THF (30 mL) was added a solution of 5-Bromo-1-chloro-2,3-difluorobenzene (CAS 1060813-07-1, 2 g, 8.79 mmol) in THF (10 mL) dropwise at-70° C. under N₂. The mixture was stirred at −70° C. for 15 min, then a solution of DMF (1.29 g, 17.59 mmol) was added at −70° C. The resulting mixture was stirred at −70° C. for 15 min. The reaction mixture was added into iced water (50 mL) and acidified to pH=4 by 1 M HCl aqueous solution, then the mixture was extracted with EA (50 mL×2). The combined organic phase was washed by brine (50 mL×2), dried (Na₂SO₄), filtered and concentrated in vacuo to give a residue, which was purified (PM31) to afford compound 2.259 (1.24 g, 4.85 mmol, 55.20% yield) as a yellow solid.

¹H NMR (400 MHz, CHCl₃-d) δ 10.26 (s, 1H), 7.58 (dd, J=5.6 Hz, 2.0 Hz, 1H). 4-bromo-6-chloro-7-fluoro-1H-indazole 2.260

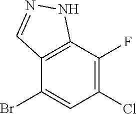 [SMILES: Fc1c(Cl)cc(Br)c2cn[nH]c12]

To a solution of compound 2.259 (1.24 g, 4.85 mmol) in dioxane (25 mL) was added N₂H₄·H₂O (770 mg, 15.07 mmol, 98% purity). The mixture was heated to 80° C. and stirred for 12 h. The reaction mixture was poured into water (60 mL) and extracted with EA (50 mL×3).

The combined organic phase was washed by brine (50 mL×2), dried (Na₂SO₄), filtered and concentrated in vacuo to afford compound 2.260 (1.2 g, 4.76 mmol, 98.10% yield) as a yellow solid.

LCMS (AM1): rt=0.461 min, (250.9 [M+2+H]⁺), 99.0% purity.

¹H NMR (400 MHz, CHCl₃-d) δ 10.80 (br s, 1H), 8.11 (d, J=3.2 Hz, 1H), 7.34 (d, J=5.6 Hz, 1H) ppm.

### 4-bromo-6-chloro-7-fluoro-1-(tetrahydro-2H-pyran-2-yl)-1H-indazole 2.261

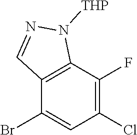

To a solution of compound 2.260 (1.23 g, 4.93 mmol) in THF (25 mL) was added DHP (1.24 g, 14.79 mmol) and TsOH (84.90 mg, 493.05 μmol) at 20° C. The mixture was stirred at 60° C. for 2 h. The reaction mixture was diluted with EA (50 mL), washed with sat. NaHCO₃ (aq.) (30 mL×2) and brine (30 mL×2). The organic phase was concentrated in vacuo to give a residue, which was purified (PM29) to afford compound 2.261 (1.2 g, 3.54 mmol, 71.87% yield) as a yellow solid.

LCMS (AM1): rt=0.673 min, (250.9 [M-THP+2+H]⁺), 98.5% purity.

### 6-chloro-7-fluoro-1-(tetrahydro-2H-pyran-2-yl)-1H-indazol-4-amine 2.262

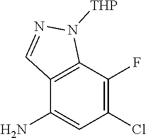

To a solution of compound 2.261 (850 mg, 2.55 mmol) and NH₃·H₂O (1.28 g, 10.19 mmol, 28% purity) in DMSO (15 mL) was added K₂CO₃ (704.32 mg, 5.10 mmol) and L-proline (117.34 mg, 1.02 mmol) followed by CuI (97.06 mg, 509.62 μmol). The mixture was degassed and purged with N₂ (×3), then the mixture was stirred at 100° C. for 12 h. The reaction mixture was poured onto water (100 mL) and extracted with EA (50 mL×3). The combined organic phase was washed by brine (40 mL×4), dried (Na₂SO₄), filtered and concentrated in vacuo to afford compound 2.262 (650 mg, crude) as a brown solid

LCMS (AM1): rt=0.503 min, (270.1 [M+H]⁺), 77.5% purity.

### 7-fluoro-6-(pyridin-4-yl)-1-(tetrahydro-2H-pyran-2-yl)-1H-indazol-4-amine 2.263

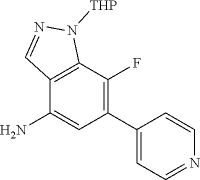

To a solution of compound 2.262 (650 mg, 2.41 mmol) and 4-pyridylboronic acid (355.49 mg, 2.89 mmol) in dioxane (15 mL) and H₂O (1.5 mL) was added Xphos Pd G4 (207.38 mg, 241.01 μmol) followed by K₂CO₃ (666.17 mg, 4.82 mmol). The mixture was degassed and purged with N₂ (×3). The mixture was stirred at 100° C. for 3 h. The reaction mixture was cooled to 20° C., diluted with EA (60 mL) and filtered. The filtrate was concentrated in vacuo to give a residue, which was purified (PM53) to afford compound 2.263 (580 mg, 1.84 mmol, 76.51% yield) as a brown solid.

LCMS (AM1): rt=0.350 min, (313.0 [M+H]⁺), 99.3% purity.

### Synthesis of Intermediate 2.265

### tert-butyl ((2-chloro-[1,1′-biphenyl]-4-yl)methyl)(3-oxo-3-((3-((6-(pyrimidin-5-yl)-1-(tetrahydro-2H-pyran-2-yl)-1H-indazol-4-yl)amino)propyl)amino)propyl)carbamate 2.265

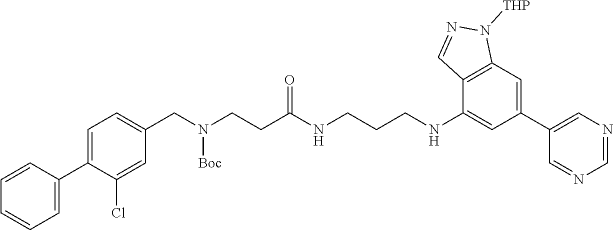

To a solution of compound 1.382 (140 mg, 193.07 μmol) in H₂O (0.2 mL) and dioxane (2 mL) was added K₂CO₃ (53.37 mg, 386.15 μmol), pyrimidin-5-yl boronic acid (28.71 mg, 231.69 μmol) and Pd(dppf)Cl₂ (14.13 mg, 19.31 μmol). The mixture was stirred at 90° C. for 0.5 h under microwave irradiation. The mixture was concentrated in vacuo to give a residue, which was purified (PM100) to afford compound 2.265 (90 mg, 116.18 μmol, 60.18% yield) as a yellow solid.

LCMS (AM3): rt=1.068 min, (724.2 [M+H]⁺), 93.50% purity.

### Synthesis of Intermediate 2.267

### 4-(4-nitro-1-(tetrahydro-2H-pyran-2-yl)-1H-indazol-6-yl)morpholine 2.266

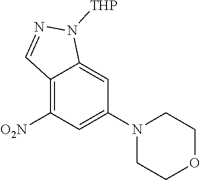

To a solution of 6-bromo-4-nitro-1-(tetrahydro-2H-pyran-2-yl)-1H-indazole (Ref: WO2019027960, 0.5 g, 1.53 mmol) in DMSO (5 mL) was added CuI (29.20 mg, 0.15 mmol), morpholine (160.27 mg, 1.84 mmol), 2-(2, 6-dimethylanilino)-2-oxo-acetic acid (59.24 mg, 0.31 mmol) and K₃PO₄ (650.84 mg, 3.07 mmol). The mixture was stirred at 110° C. for 12 h under N₂. H₂O (50 mL) was added and extracted by EA (30 mL×2). The combined organic phase was dried (Na₂SO₄), filtered and concentrated in vacuo to give a residue, which was purified (PM45) to afford compound 2.266 (200 mg, 39.2% yield) as a red oil.

LCMS (AM3): rt=0.959 min, (333.6 [M+H]⁺), 100% purity.

### 6-morpholino-1-(tetrahydro-2H-pyran-2-yl)-1H-indazol-4-amine 2.267

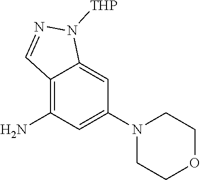

To a solution of compound 2.266 (190 mg, 0.57 mmol) in MeOH (10 mL) was added Pd/C (200 mg, 0.057 mmol, 10% purity) under N₂ atmosphere. The suspension was degassed and purged with H₂ (×3). The mixture was stirred at 25° C. for 1 h under H₂ (15 Psi). The catalyst was filtered and the filtrate was concentrated in vacuo to give a residue, which was purified (PM161) to afford compound 2.267 (180 mg, 91.9% yield) as a light-yellow oil.

LCMS (AM3): rt=0.662 min, (303.6 [M+H]⁺), 88.3% purity.

### Synthesis of Intermediate 2.270

### 6-(2-methyl-1H-imidazol-1-yl)-4-nitro-1-(tetrahydro-2H-pyran-2-yl)-1H-indazole 2.269

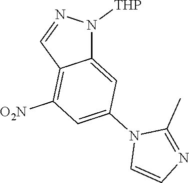

To a solution of compound 1.943 (565 mg, 1.94 mmol) in DMF (1 mL) was added Cu(OAc)₂ (423.09 mg, 2.33 mmol), 2-methyl-1H-imidazole (239.06 mg, 2.91 mmol), 4A molecular sieve (1 g, 1.94 mmol) and TEA (785.69 mg, 7.76 mmol). The mixture was stirred at 65° C. for 12 h under oxygen. The mixture was filtered and the filtrate was concentrated in vacuo to give a residue, which was purified (PM44) to afford compound 2.269 (200 mg, 28.7% yield) as a yellow solid.

LCMS (AM3): rt=0.223 min, (327.9 [M+H]⁺), 91.4% purity.

### 6-(2-methyl-1H-imidazol-1-yl)-1-(tetrahydro-2H-pyran-2-yl)-1H-indazol-4-amine 2.270

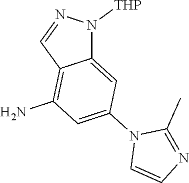

To a solution of NiCl₂·6H₂O (72.61 mg, 0.30 mmol) in MeOH (5 mL) was added NaBH₄ (11.56 mg, 0.31 mmol) at 0° C. After 5 min, compound 2.269 (200 mg, 0.61 mmol) and NaBH₄ (69.35 mg, 1.83 mmol) was added. The mixture was stirred at 0° C. for 25 min. The mixture was filtered and the filtrate was concentrated in vacuo to give a residue, which was purified (PM163) to afford compound 2.270 (180 mg, 88.1% yield) as a yellow oil.

LCMS (AM3): rt=0.489 min, (297.9 [M+H]⁺), 74.3% purity.

### Synthesis of Intermediate 2.277

### 6-(3,6-dihydro-2H-pyran-4-yl)-4-nitro-1-(tetrahydro-2H-pyran-2-yl)-1H-indazole 2.272

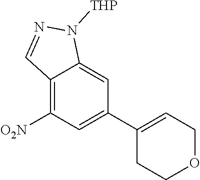

To a solution of 6-bromo-4-nitro-1-(tetrahydro-2H-pyran-2-yl)-1H-indazole (WO2019027960, 1 g, 3.07 mmol) in dioxane (10 mL) and H₂O (1 mL) was added K₃PO₄ (1.30 g, 6.13 mmol), 2-(3,6-dihydro-2H-pyran-4-yl)-4,4,5,5-tetramethyl-1,3,2-dioxaborolane (772.94 mg, 3.68 mmol) and Pd(dppf)Cl₂ (224.35 mg, 0.31 mmol). The mixture was stirred at 80° C. for 12 h under N₂.

The mixture was concentrated in vacuo to give a residue, which was purified (PM45) to afford compound 2.272 (2.8 g, 92.4% yield) as a yellow solid. 1-(tetrahydro-2H-pyran-2-yl)-6-(tetrahydro-2H-pyran-4-yl)-1H-indazol-4-amine 2.273

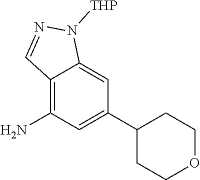

To a solution of compound 2.272 (2.8 g, 8.50 mmol) in MeOH (50 mL) was added Pd/C (2 g, 0.85 mmol, 10% purity) under N₂ atmosphere. The mixture was degassed and purged with H₂ (×3). The mixture was stirred at 25° C. for 12 h under H₂ (15 Psi). The catalyst was filtered and the filtrate was concentrated in vacuo to afford compound 2.273 (2.4 g, 90.4% yield) as a black-brown solid.

LCMS (AM3): rt=0.800 min, (302.2 [M+H]⁺), 96.5% purity.

### tert-butyl (3-oxo-3-((1-(tetrahydro-2H-pyran-2-yl)-6-(tetrahydro-2H-pyran-4-yl)-1H-indazol-4-yl)amino)propyl)carbamate 2.274

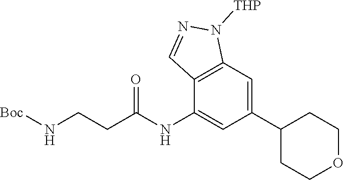

To a solution of compound 2.273 (2 g, 6.64 mmol) in pyridine (50 mL) was added EDCI (12.72 g, 66.36 mmol) and 3-((tert-butoxycarbonyl)amino)propanoic acid (1.51 g, 7.96 mmol).The mixture was stirred at 100° C. for 12 h. The reaction mixture was concentrated in vacuo to give a residue, which was purified (PM89) to afford compound 2.274 (2 g, 63.3% yield) as a yellow solid.

LCMS (AM3): rt=0.935 min, (473.2 [M+H]⁺), 99.3% purity.

### tert-butyl (3-((1-(tetrahydro-2H-pyran-2-yl)-6-(tetrahydro-2H-pyran-4-yl)-1H-indazol-4-yl)amino)propyl)carbamate 2.275

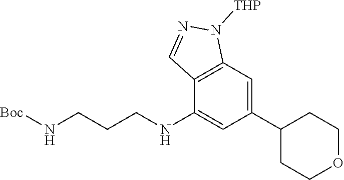

To a solution of compound 2.274 (2 g, 4.23 mmol, 1 eq) in THF (30 mL) was added BH₃-Me₂S (10 M, 4.23 mL) at 0° C. The mixture was stirred at 25° C. for 2 h. MeOH (30 mL) was slowly dropped into the mixture until no gas was evolved, then the mixture was concentrated in vacuo to give a residue, which was purified (PM165) to afford compound 2.275 (1.3 g, 42.0% yield) as a light-red solid.

LCMS (AM3): rt=0.876 min, (459.3 [M+H]⁺), 61.8% purity.

### N1-(6-(tetrahydro-2H-pyran-4-yl)-1H-indazol-4-yl)propane-1,3-diamine 2.276

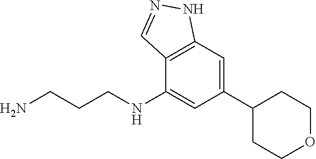 [SMILES: NCCCNc1cc(C2CCOCC2)cc2[nH]ncc12]

A solution of compound 2.275 (900 mg, 1.23 mmol) in HCl/dioxane (4 M, 22.50 mL) was stirred at 25° C. for 2 h. The mixture was concentrated in vacuo to give a residue, which was triturated with EA (30 mL) at 25° C. for 30 min. The mixture was filtered to give compound 2.276 (500 mg, crude, HCl salt) as a light-yellow solid.

LCMS (AM8): rt=1.510 min, (275.1 [M+H]⁺), 98.5% purity.

### tert-butyl ((2-chloro-[1,1′-biphenyl]-4-yl)methyl)(3-oxo-3-((3-((6-(tetrahydro-2H-pyran-4-yl)-1H-indazol-4-yl)amino)propyl)amino)propyl)carbamate 2.277

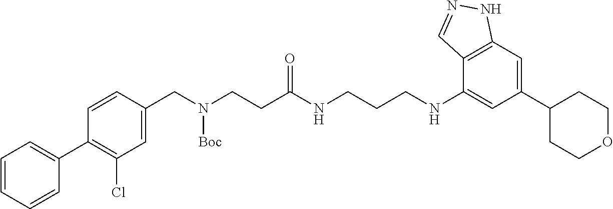 [SMILES: CC(C)(C)OC(=O)N(CCC(=O)NCCCNc1cc(C2CCOCC2)cc2[nH]ncc12)Cc1ccc(-c2ccccc2)c(Cl)c1]

To a solution of intermediate D (487.67 mg, 1.25 mmol) in pyridine (10 mL) was added EDCI (999.12 mg, 5.21 mmol) and compound 2.276 (0.4 g, 1.04 mmol, HCl salt). The mixture was stirred at 100° C. for 12 h. The reaction mixture was concentrated in vacuo to give a residue, which was purified (PM166) to afford compound 2.277 (130 mg, 18.1% yield) as a yellow solid.

LCMS (AM3): rt=0.919 min, (646.1 [M+H]⁺), 93.8% purity.

### Synthesis of Intermediate 2.285

### 6-bromo-4-fluoro-1-trityl-1H-benzo[d][1,2,3]triazole 2.280

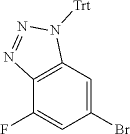

To a solution of compound 2.040 (5 g, 23.15 mmol) in THF (75 mL) was added NaH (1.02 g, 25.46 mmol, 60% purity) at 0° C. The mixture was stirred at 0° C. for 0.5 h, then TrtCl (6.78 g, 24.30 mmol) was added and the mixture was stirred at 25° C. for 3 h. The reaction mixture was quenched with water (50 mL) and extracted with EA (100 mL×2). The combined organic layers were washed (brine, 100 mL×2), dried (Na₂SO₄), filtered and concentrated in vacuo to afford compound 2.280 (10.5 g, 22.91 mmol, 98.97% yield) as an off-white solid, which was used for the next step without further purification.

### tert-butyl(4-((1-(6-bromo-1-trityl-1H-benzo[d][1,2,3]triazol-4-yl)azetidin-3-yl)oxy)butyl)carbamate 2.281

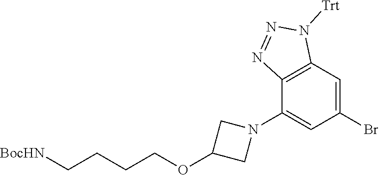

To a solution of tert-butyl (4-(azetidin-3-yloxy)butyl)carbamate (Ref: WO2022185041, 3.84 g, 15.71 mmol) and compound 2.280 (6 g, 13.09 mmol) in DMF (40 mL) was added K₂CO₃ (3.62 g, 26.18 mmol). The mixture was stirred at 75° C. for 7 h. The reaction mixture was quenched with water (60 mL) and extracted with EA (100 mL×2). The combined organic layers were washed (brine, 50 mL×2), dried (Na₂SO₄), filtered and concentrated in vacuo to give a residue, which was purified (PM55) to afford compound 2.281 (6.46 g, 9.46 mmol, 72.29% yield) as a yellow oil.

LCMS (AM3): rt=1.172 min, (706.0 [M+H]⁺), 79.3% purity

### tert-butyl(4-((1-(6-(4,4,5,5-tetramethyl-1,3,2-dioxaborolan-2-yl)-1-trityl-1H-benzo[d][1,2,3]triazol-4-yl)azetidin-3-yl)oxy)butyl)carbamate 2.282

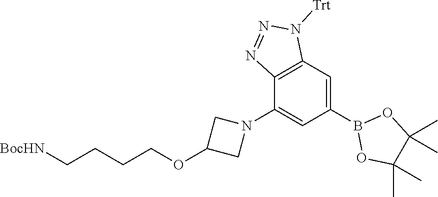

To a mixture of compound 2.281 (1.35 g, 1.98 mmol) and 4,4,4′,4′,5,5,5′,5′-octamethyl-2,2′-bi(1,3,2-dioxaborolane) (602.62 mg, 2.37 mmol) in 1,4-dioxane (10 mL) was added KOAc (388.16 mg, 3.96 mmol) and Pd(dppf)Cl₂ (144.70 mg, 197.76 μmol). The mixture was stirred at 85° C. for 16 h under N₂. Water (50 mL) was added and extracted with EA (50 mL×2). The combined organic layers were washed (brine, 30 mL×2), dried (Na₂SO₄), filtered and concentrated in vacuo to give a residue, which was purified (PM36) to afford compound 2.282 (1.35 g, 1.85 mmol, 93.55% yield) as a light-yellow oil.

LCMS (AM3): rt=1.282 min, (752.7 [M+Na]⁺), 81.68% purity

### tert-butyl(4-((1-(6-(pyridazin-4-yl)-1-trityl-1H-benzo[d][1,2,3]triazol-4-yl)azetidin-3-yl)oxy)butyl)carbamate 2.283

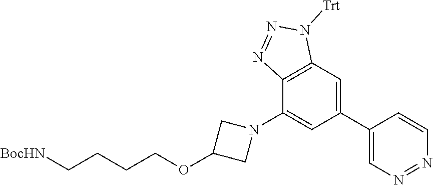

To a mixture of 4-bromopyridazine (421.63 mg, 1.76 mmol, HBr salt) and compound 2.282 (1.35 g, 1.85 mmol) in 1,4-dioxane (13.5 mL) and H₂O (1.5 mL) was added K₂CO₃ (767.08 mg, 5.55 mmol) and Pd(dppf)Cl₂ (135.37 mg, 185.00 μmol) at 25° C. The mixture was stirred at 85° C. for 16 h under N₂. Water (50 mL) was added and extracted with EA (50 mL×2). The combined organic layers were washed (brine, 50 mL×2), dried (Na₂SO₄), filtered and concentrated in vacuo to give a residue, which was purified (PM30) to afford compound 2.283 (800 mg, 1.17 mmol, 63.42% yield) as a yellow solid

LCMS (AM3): rt=1.082 min, (682.3 [M+H]⁺), 75.23% purity

### tert-butyl(4-((1-(6-(pyridazin-4-yl)-1H-benzo[d][1,2,3]triazol-4-yl)azetidin-3-yl)oxy)butyl)carbamate 2.284

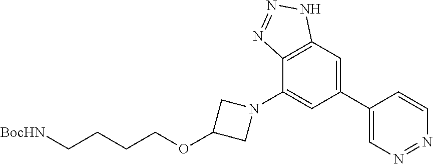 [SMILES: CC(C)(C)OC(=O)NCCCCOC1CN(c2cc(-c3ccnnc3)cc3[nH]nnc23)C1]

To a solution of compound 2.283 (570 mg, 835.99 μmol) in DCM (60 mL) was added Et₃SiH (194.41 mg, 1.67 mmol, 267.05 μL) followed by TFA (1.91 g, 16.72 mmol, 1.24 mL) at 0° C. under N₂. The mixture was stirred at 25° C. for 0.5 h. The reaction mixture was concentrated in vacuo to afford compound 2.284 (460 mg, 831.03 μmol, 99.41% yield, TFA salt) as a brown oil, which was used for the next step directly.

LCMS (AM3): rt=0.863 min, (440.3 [M+H]⁺), 48% purity

### 4-((1-(6-(pyridazin-4-yl)-1H-benzo[d][1,2,3]triazol-4-yl)azetidin-3-yl)oxy)butan-1-amine 2.285

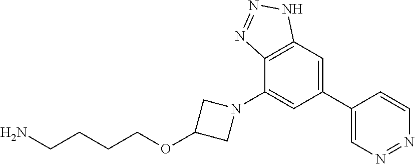 [SMILES: NCCCCOC1CN(c2cc(-c3ccnnc3)cc3[nH]nnc23)C1]

To a solution of compound 2.284 (460 mg, 831.03 μmol, TFA salt) in DCM (20 mL) was added TFA (3.08 g, 27.01 mmol, 2 mL) and the mixture was stirred at 25° C. for 1 h. The reaction mixture was concentrated in vacuo to give a residue, which was purified (PM88) to afford compound 2.285 (400 mg, 830.27 μmol) as a brown oil

LCMS (AM3): rt=0.641 min, (340.4 [M+H]⁺), 96.69% purity

### Synthesis of Intermediate 2.289

### tert-butyl (3-(3-iodopropoxy)propyl)carbamate 2.287

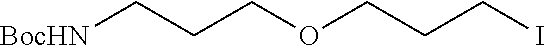 [SMILES: CC(C)(C)OC(=O)NCCCOCCCI]

To a mixture of tert-butyl N-[3-(3-hydroxypropoxy)propyl]carbamate (CAS 1312905-31-9, 500 mg, 2.14 mmol) in DCM (10 mL) was added imidazole (291.81 mg, 4.29 mmol) and PPh₃ (1.12 g, 4.29 mmol) in one portion at 0° C. under N₂, then 12 (1.09 g, 4.29 mmol) was added at 0° C. and the mixture was stirred at 25° C. for 12 h. The mixture was concentrated in vacuo to give a residue and saturated Na₂SO₃ (30 mL) solution was added. The aqueous phase was extracted with EA (50 mL×3). The combined organic phase was washed (brine, 50 mL×1), dried (Na₂SO₄), filtered and concentrated in vacuo to give a residue, which was purified (PM46) to afford compound 2.287 (560 mg, 1.63 mmol, 76.14% yield) as a yellow oil.

¹H NMR (400 MHz, MeOH-d₄) δ 3.50 (t, J=4.4 Hz, 4H), 3.33 (t, J=6.8 Hz, 2H), 3.14 (t, J=6.8 Hz, 2H), 2.07-2.01 (m, 2H), 1.75-1.72 (m, 2H), 1.45 (s, 9H) ppm

### tert-butyl(3-(3-((1-(tetrahydro-2H-pyran-2-yl)-6-(4H-1,2,4-triazol-4-yl)-1H-indazol-4-yl)oxy)propoxy)propyl)carbamate 2.288

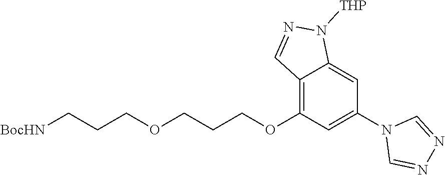

To a mixture of compound 1.360 (200 mg, 701.01 μmol) in DMF (2 mL) was added K₂CO₃ (193.77 mg, 1.40 mmol) and compound 2.287 (360.88 mg, 1.05 mmol). The mixture was stirred at 80° C. for 12 h. The mixture was concentrated in vacuo to give a residue, which was purified (PM167) to afford compound 2.288 (200 mg, 331.61 μmol, 47.30% yield) as a brown gum.

LCMS (AM3): rt=0.918 min, (501.2 [M+H]⁺), 83.28% purity.

### 3-(3-((6-(4H-1,2,4-triazol-4-yl)-1H-indazol-4-yl)oxy)propoxy)propan-1-amine 2.289

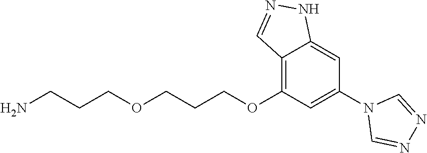 [SMILES: NCCCOCCCOc1cc(-n2cnnc2)cc2[nH]ncc12]

A mixture of compound 2.288 (200 mg, 331.61 μmol) in HCl/dioxane (4 M, 5.53 mL) was stirred at 20° C. for 2 h. The mixture was concentrated in vacuo to afford compound 2.289 (200 mg, crude, HCl salt) as a brown solid, which was used without further purification.

LCMS (AM3): rt=0.549 min, (317.4 [M+H]⁺), 98.32% purity.

### Synthesis of Intermediate 2.294

### 3-((3-chloro-4-(trifluoromethoxy)benzyl)amino)propan-1-ol 2.291

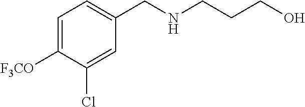 [SMILES: OCCCNCc1ccc(OC(F)(F)F)c(Cl)c1]

To a solution of 3-aminopropan-1-ol (668.48 mg, 8.90 mmol) in MeOH (10 mL) was added 3-chloro-4-(trifluoromethoxy)benzaldehyde (CAS 83279-39-4, 1 g, 4.45 mmol). The resulting mixture was stirred at 20° C. for 13 h, then NaBH(OAc)₃ (943.79 mg, 4.45 mmol) was added. The mixture was stirred for 2 h. The mixture was filtered and the filtrate was concentrated in vacuo to give a residue, which was purified (PM90) to afford compound 2.291 (230 mg, 578.33 μmol, 13.00% yield, TFA salt) as a yellow oil.

LCMS (AM3): rt=0.840 min, (284.0 [M+H]⁺), 86.50% purity.

### Tert-butyl (3-chloro-4-(trifluoromethoxy)benzyl)(3-hydroxypropyl)carbamate 2.292

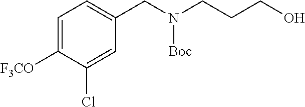 [SMILES: CC(C)(C)OC(=O)N(CCCO)Cc1ccc(OC(F)(F)F)c(Cl)c1]

To a solution of compound 2.291 (230 mg, 578.33 μmol, TFA salt) in THF (10 mL) was added NaHCO₃ (72.88 mg, 867.49 μmol), Boc₂O (151.46 mg, 694.00 μmol). The reaction mixture was stirred at 20° C. for 12 h. The reaction mixture was diluted with H₂O (20 mL) and extracted with EA (20 mL×3). The organic layer was washed (brine, 30 mL×2), dried (Na₂SO₄), filtered and concentrated in vacuo to afford compound 2.292 (615 mg, crude) as a yellow oil.

LCMS (AM3): rt=1.000 min, (284.5 [M-100+H]⁺), 94.46% purity.

### Tert-butyl (3-chloro-4-(trifluoromethoxy)benzyl)(3-oxopropyl)carbamate 2.293

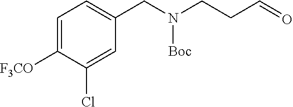 [SMILES: CC(C)(C)OC(=O)N(CCC=O)Cc1ccc(OC(F)(F)F)c(Cl)c1]

A mixture of compound 2.292 (590 mg, 1.54 mmol) and DMP (800 mg, 1.89 mmol) in DCM (15 mL) was stirred at 16° C. for 15 h. The reaction mixture was filtered and the filtrate was concentrated in vacuo to give a residue, which was purified (PM11) to afford compound 2.293 (370 mg, 59.26% yield) as a colourless oil.

LCMS (AM3): rt=1.038 min, (326.2 [M-C₄H₃+H]⁺), 94.47% purity.

### Tert-butyl (3-((3-((6-(4H-1,2,4-triazol-4-yl)-1H-indazol-4-yl)amino)-3-oxopropyl)amino)propyl)(3-chloro-4-(trifluoromethoxy)benzyl)carbamate 2.294

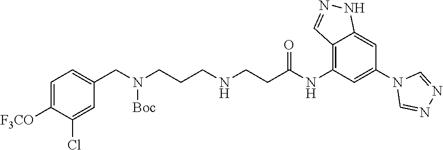 [SMILES: CC(C)(C)OC(=O)N(CCCNCCC(=O)Nc1cc(-n2cnnc2)cc2[nH]ncc12)Cc1ccc(OC(F)(F)F)c(Cl)c1]

A mixture of compound 2.293 (40 mg, 104.77 μmol), compound 1.285 (40 mg, 129.98 μmol, HCl salt) and DIPEA (459.30 μmol) in MeOH (4 mL) was stirred at 25° C. for 16 h, then NaBH₃CN (40 mg, 636.54 μmol) was added and the reaction was stirred for 1 h. The reaction mixture was concentrated in vacuo to give a residue, which was purified (PM168) to afford compound 2.294 (30 mg, 38.12% yield) as a white solid.

LCMS (AM3): rt=0.872 min, (637.2 [M+H]⁺), 100% purity.

### Synthesis of Intermediate 2.298

### 3-(3-((tert-butoxycarbonyl)amino)propoxy)propanoic acid 2.296

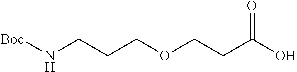 [SMILES: CC(C)(C)OC(=O)NCCCOCCC(=O)O]

To a mixture of tert-butyl N-[3-(3-hydroxypropoxy)propyl]carbamate (CAS 1312905-31-9, 1.1 g, 4.71 mmol) in DCM (10 mL) and H₂O (10 mL) was added TEMPO (220 mg, 1.40 mmol) followed by [acetoxy(phenyl)-iodanyl] acetate (3.04 g, 9.43 mmol). The reaction mixture was stirred at 25° C. for 3 h. The reaction mixture was poured to NaOH solution (1 N, 80 mL). The resultant mixture was extracted with EA (30 mL×3). The pH of the aqueous phase was adjusted to ˜3 with conc. HCl, then the mixture was extracted with EA (30 mL×3). The combined organic phase was washed (brine, 50 mL×2), dried (Na₂SO₄), filtered and concentrated in vacuo to afford compound 2.296 (850 mg, 3.44 mmol, 72.90% yield) as a brown oil.

¹H NMR (400 MHz, DMSO-d₆) δ 6.74 (t, J=5.6 Hz, 1H), 3.57 (t, J=4.4 Hz, 2H), 3.47 (t, J=7.6 Hz, 2H), 2.99-2.87 (m, 2H), 2.42 (t, J=6.0 Hz, 2H), 1.63-1.48 (m, 2H), 1.37 (s, 9H) ppm

### tert-butyl (3-(3-((6-(4H-1,2,4-triazol-4-yl)-1H-indazol-4-yl)amino)-3-oxopropoxy)propyl)carbamate 2.297

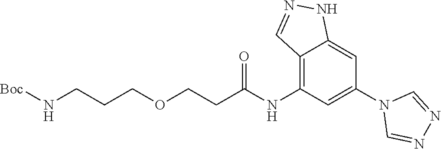 [SMILES: CC(C)(C)OC(=O)NCCCOCCC(=O)Nc1cc(-n2cnnc2)cc2[nH]ncc12]

A mixture of compound 2.296 (400 mg, 1.62 mmol), compound 1.273 (400 mg, 1.41 mmol) and EDCI (808 mg, 4.21 mmol) in pyridine (10 mL) was stirred at 80° C. for 16 h. The reaction mixture was concentrated in vacuo to give a residue, which was purified (PM169) to afford compound 2.297 (250 mg, 34.60% yield) as a brown solid.

LCMS (AM3): rt=0.861 min, (514.4 [M+H]⁺), 100% purity.

### N-(6-(4H-1,2,4-triazol-4-yl)-1H-indazol-4-yl)-3-(3-aminopropoxy)propanamide 2.298

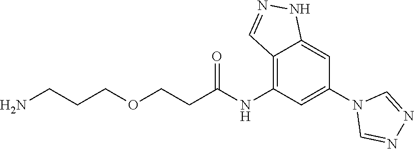 [SMILES: NCCCOCCC(=O)Nc1cc(-n2cnnc2)cc2[nH]ncc12]

A mixture of compound 2.297 (250 mg, 486.77 μmol) in HCl/dioxane (4 M, 10 mL) was stirred at 25° C. for 1 h. The reaction mixture was concentrated in vacuo to afford compound 2.298 (180 mg, crude, HCl salt) as a brown solid, which was used for the next step directly without further purification.

LCMS (AM3): rt=0.229 min, (330.1 [M+H]⁺), ˜70% purity.

### Synthesis of Intermediate 2.304

### 4-fluoro-1-((2-(trimethylsilyl)ethoxy)methyl)-1H-benzo[d][1,2,3]triazol-6-amine 2.300

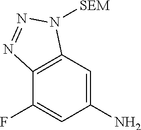 [SMILES: C[Si](C)(C)CCOCn1nnc2c(F)cc(N)cc21]

To a solution of 6-bromo-4-fluoro-1-((2-(trimethylsilyl)ethoxy)methyl)-1H-benzo[d][1,2,3]triazole (9 g, 25.99 mmol, Ref: WO202256449) in DMSO (120 mL) was added NH₃·H₂O (10.93 g, 77.97 mmol, 12.01 mL, 25% purity), K₂CO₃ (10.78 g, 77.97 mmol), CuI (990.01 mg, 5.20 mmol) and (2S,4S)-4-hydroxypyrrolidine-2-carboxylic acid (1.36 g, 10.40 mmol). The reaction mixture was stirred at 80° C. for 12 h. The reaction mixture was diluted with H₂O (150 mL) and extracted with EA (200 mL×2). The organic layer was washed (brine, 200 mL), dried (Na₂SO₄), filtered and concentrated in vacuo to give a residue, which was purified (PM25) to afford compound 2.300 (3.32 g, 11.76 mmol, 45.23% yield) as a red oil.

LCMS (AM3): rt=0.914 min, (283.1 [M+H]⁺), 99.20% purity.

### 4-fluoro-6-(4H-1,2,4-triazol-4-yl)-1-((2-(trimethylsilyl)ethoxy)methyl)-1H-benzo[d][1,2,3]triazole 2.301

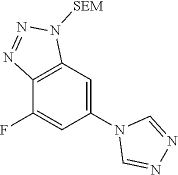 [SMILES: C[Si](C)(C)CCOCn1nnc2c(F)cc(-n3cnnc3)cc21]

To a solution of N-formamidoformamide (5.15 g, 58.43 mmol) and compound 2.300 (3.3 g, 11.69 mmol) in pyridine (75 mL) was added TEA (8.28 g, 81.80 mmol, 11.39 mL) and TMSCl (19.04 g, 175.29 mmol). The mixture was stirred at 120° C. for 16 h. The reaction mixture was diluted with water (50 mL) and extracted with EA (100 mL×2). The combined organic layers were washed (brine, 50 mL×2), dried (Na₂SO₄), filtered and concentrated in vacuo to give a residue, which was purified (PM23) to afford compound 2.301 (2.2 g, 6.58 mmol, 56.29% yield) as a grey solid

LCMS (AM3): rt=0.809 min, (335.1 [M+H]⁺), 99.53% purity.

### tert-butyl (4-((1-(6-(4H-1,2,4-triazol-4-yl)-1-((2-(trimethylsilyl)ethoxy)methyl)-1H-benzo[d][1,2,3]triazol-4-yl)azetidin-3-yl)oxy)butyl)carbamate 2.302

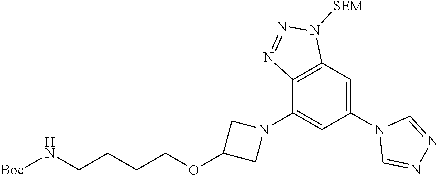 [SMILES: CC(C)(C)OC(=O)NCCCCOC1CN(c2cc(-n3cnnc3)cc3c2nnn3COCC[Si](C)(C)C)C1]

To a solution of tert-butyl (4-(azetidin-3-yloxy)butyl)carbamate (WO2022185041, 109.59 mg, 448.53 μmol) and compound 2.301 (100 mg, 299.02 μmol) in DMF (2 mL) was added K₂CO₃ (82.66 mg, 598.04 μmol). The mixture was stirred at 80° C. for 3 h. The reaction mixture was diluted with water (20 mL) and extracted with EA (30 mL×2). The combined organic layers were washed (brine, 30 mL×2), dried (Na₂SO₄), filtered and concentrated in vacuo to give a residue, which was purified (PM90) to afford compound 2.302 (95 mg, 170.02 μmol, 56.86% yield) as a brown solid

LCMS (AM3): rt=0.975 min, (559.2 [M+H]⁺), 66.88% purity.

### tert-butyl (4-((1-(6-(4H-1,2,4-triazol-4-yl)-1H-benzo[d][1,2,3]triazol-4-yl)azetidin-3-yl)oxy)butyl)carbamate 2.303

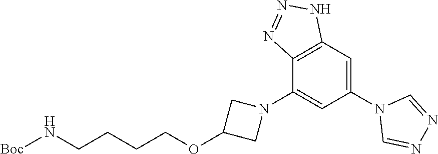 [SMILES: CC(C)(C)OC(=O)NCCCCOC1CN(c2cc(-n3cnnc3)cc3[nH]nnc23)C1]

A mixture of compound 2.032 (2.16 g, 3.87 mmol) and TBAF in THF (1 M, 25 mL) was stirred at 65° C. for 80 min. The reaction mixture was concentrated in vacuo to give a residue, which was purified (PM23) then further purified (PM170) to afford compound 2.303 (1.29 g, 3.01 mmol, 77.88% yield) as a yellow solid.

LCMS (AM3): rt=0.805 min, (429.2 [M+H]⁺), 96.84% purity.

### 4-((1-(6-(4H-1,2,4-triazol-4-yl)-1H-benzo[d][1,2,3]triazol-4-yl)azetidin-3-yl)oxy)butan-1-amine 2.304

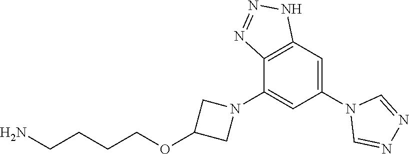 [SMILES: NCCCCOC1CN(c2cc(-n3cnnc3)cc3[nH]nnc23)C1]

To a solution of compound 2.303 (0.1 g, 233.38 μmol) in DCM (3 mL) was added TFA (4.05 mmol, 0.3 mL). The reaction mixture was stirred at 25° C. for 0.5 h. The reaction mixture was concentrated in vacuo to give a residue, which was purified (PM170) to afford compound 2.304 (80 mg, 197.87 μmol, 84.78% yield, FA salt) as a yellow solid.

LCMS (AM3): rt=0.294 min, (329.2 [M+H]⁺), 92.60% purity.

### Synthesis of Intermediate 2.306

### 6-(2-aminopyridin-4-yl)-1-(tetrahydro-2H-pyran-2-yl)-1H-indazol-4-amine 2.305

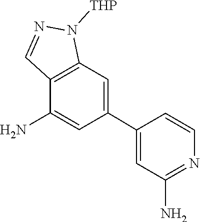

To a mixture of compound 1.952 (630 mg, 1.84 mmol) and 4-chloropyridin-2-amine (CAS 19798-80-2, 247.77 mg, 1.93 mmol) in dioxane (10 mL) and H₂O (0.1 mL) was added Cs₂CO₃ (897.07 mg, 2.75 mmol) followed by Pd(dppf)Cl₂ (134.31 mg, 183.55 μmol). The mixture was heated to 100° C. and stirred for 12 h. The reaction mixture was diluted with DCM (30 mL) and filtered. The filtrate was concentrated in vacuo to give a residue, which was purified (PM22) to afford compound 2.305 (250 mg, 750.89 μmol, 40.91% yield) as a yellow solid.

LCMS (AM12): rt=0.340 min, (310.1 [M+H]⁺), 92.92% purity.

### 6-(2-aminopyridin-4-yl)-1H-indazol-4-amine 2.306

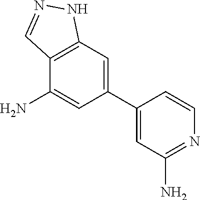 [SMILES: Nc1cc(-c2cc(N)c3cn[nH]c3c2)ccn1]

A mixture of compound 2.305 (250 mg, 808.11 μmol) in HCl/dioxane (4 M, 3 mL) was stirred at 20° C. for 1 h. The mixture was concentrated in vacuo to afford the compound 2.306 (290 mg, crude, HCl salt) as a yellow solid.

LCMS (AM12): rt=0.256 min, (226.1 [M+H]⁺), 91.15% purity.

### Synthesis of Intermediate 2.313

### ethyl 6-chloro-1-(tetrahydro-2H-pyran-2-yl)-1H-pyrazolo[3,4-b]pyridine-4-carboxylate 2.309

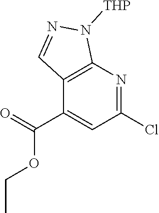

To a solution of ethyl 6-chloro-1H-pyrazolo[3,4-b]pyridine-4-carboxylate (5.2 g, 23.05 mmol, US2013/150340) in THF (60 mL) was added DHP (5.82 g, 69.14 mmol) followed by TsOH (396.86 mg, 2.30 mmol). The mixture was heated to 50° C. and stirred for 2 h. The reaction mixture was concentrated in vacuo to give a residue, which was purified (PM48) to afford compound 2.309 (7.2 g, 22.87 mmol, 99.25% yield) as a yellow solid.

LCMS (AM12): rt=0.591 min, (332.0 [M+Na]l), 98.47% purity.

### ethyl 6-(pyridin-4-yl)-1-(tetrahydro-2H-pyran-2-yl)-1H-pyrazolo[3,4-b]pyridine-4-carboxylate 2.310

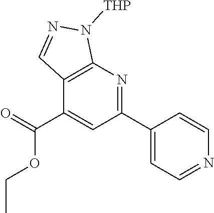

To a solution of compound 2.309 (3 g, 9.69 mmol) in dioxane (50 mL) and H₂O (5 mL) was added pyridin-4-ylboronic acid (1.43 g, 11.62 mmol) and K₂CO₃ (2.01 g, 14.53 mmol) followed by Pd(dppf)Cl₂ (708.68 mg, 968.53 μmol) at 20° C. The mixture was degassed and purged with N₂ (×3), the mixture was heated to 80° C. and stirred for 3 h. The reaction mixture was cooled to 20° C., diluted with EA (50 mL) and filtered. The filtrate was concentrated in vacuo to give a residue, which was purified (PM33) to afford compound 2.310 (2.23 g, 5.99 mmol, 61.88% yield) as a yellow solid.

LCMS (AM12): rt=0.442 min, (353.1 [M+H]⁺), 94.70% purity.

### 6-(pyridin-4-yl)-1-(tetrahydro-2H-pyran-2-yl)-1H-pyrazolo[3,4-b]pyridine-4-carboxylic acid 2.311

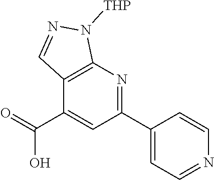

To a solution of compound 2.310 (1 g, 2.84 mmol) in MeOH (15 mL) was added NaOH (113.50 mg, 2.84 mmol) and H₂O (5 mL). The mixture was stirred at 2° C. for 2 h. The reaction mixture was concentrated in vacuo. The residue was dissolved in H₂O (40 mL) and acidified to pH=6 (1 N HCl (aq.)). The mixture was filtered. The filter cake was dried under vacuum to afford compound 2.311 (647 mg, 1.96 mmol, 69.11% yield) as an off-white solid.

LCMS (AM11): rt=0.304 min, (325.1 [M+H]⁺), 98.31% purity.

¹H NMR (400 MHz, DMSO-d₆) δ 14.19 (br s, 1H), 8.78 (d, J=5.6 Hz, 2H), 8.49 (s, 1H), 8.35 (s, 1H), 8.23 (d, J=5.6 Hz, 2H), 6.23 (d, J=8.8 Hz, 1H), 4.05-3.94 (m, 1H), 3.86-3.70 (m, 1H), 2.73-2.56 (m, 1H), 2.13-1.95 (m, 2H), 1.91-1.78 (m, 1H), 1.68-1.52 (m, 2H) ppm.

### tert-butyl (6-(pyridin-4-yl)-1-(tetrahydro-2H-pyran-2-yl)-1H-pyrazolo[3,4-b]pyridin-4-yl)carbamate 2.312

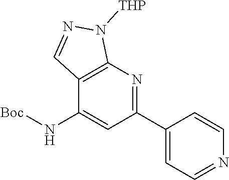

To a solution of compound 2.311 (547 mg, 1.69 mmol) in toluene (18 mL) was added DPPA (510.55 mg, 1.86 mmol) and TEA (426.65 mg, 4.22 mmol) followed by t-BuOH (2.29 g, 30.94 mmol). The mixture was heated to 105° C. and stirred for 12 h under N₂. The reaction mixture was cooled to 20° C., poured onto H₂O (40 mL) and extracted with EA (60 mL×3). The combined organic phases were washed (brine, 40 mL×2), dried (Na₂SO₄), filtered and concentrated in vacuo to give a residue, which was purified (PM32) to afford compound 2.312 (479 mg, 1.21 mmol, 71.55% yield) as a white solid.

LCMS (AM12): rt=0.458 min, (396.1 [M+H]⁺), 99.62% purity.

### 6-(pyridin-4-yl)-1H-pyrazolo[3,4-b]pyridin-4-amine 2.313

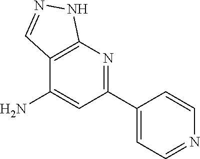 [SMILES: Nc1cc(-c2ccncc2)nc2[nH]ncc12]

A solution of compound 2.312 (660 mg, 1.67 mmol) in HCl/MeOH (4 M, 20 mL) was stirred at 20° C. for 15 h. The mixture was concentrated in vacuo to give a residue. The residue was dissolved in H₂O (20 mL) and basified to pH=9 (sat. NaHCO₃ (aq.)). The precipitated solid was filtered and washed with H₂O (2 mL×2). The filter cake was collected and dried under vacuum to afford compound 2.313 (240 mg, crude) as a grey solid.

LCMS (AM11): rt=0.318 min, (212.2 [M+H]⁺), 92.08% purity.

### Synthesis of Intermediate 2.317

### 6-(6-chloropyridazin-4-yl)-4-nitro-1-(tetrahydro-2H-pyran-2-yl)-1H-indazole 2.315

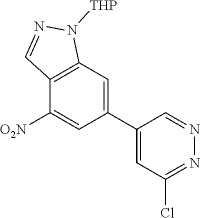

To a solution of compound 1.943 (607 mg, 1.63 mmol) and 3,5-dichloropyridazine (290.76 mg, 1.95 mmol) in dioxane (12 mL) and H₂O (1.2 mL) was added K₃PO₄ (863.09 mg, 4.07 mmol) followed by Pd(t-Bu₃P)₂ (124.68 mg, 243.96 μmol). The mixture was degassed and purged with N₂ (×3), heated to 70° C. and stirred for 2 h. The reaction mixture was cooled to 20° C., diluted with EA (40 mL) and filtered. The filtrate was concentrated in vacuo to give a residue, which was triturated with MTBE (20 mL) at 20° C. and filtered. The filter cake was collected and dried under vacuum to afford compound 2.315 (423 mg, 990.46 μmol, 60.90% yield) as a brown solid.

LCMS (AM11): rt=0.478 min, (360.1 [M+H]⁺), 84.24% purity.

¹H NMR (400 MHz, CHCl₃-d) δ 9.51 (s, 1H), 8.72 (s, 1H), 8.42 (s, 1H), 8.30 (s, 1H), 7.84 (d, J=2.0 Hz, 1H), 5.94 (dd, J=8.0 Hz, 2.4 Hz, 1H), 4.03-3.96 (m, 1H), 3.86-3.78 (m, 1H), 2.61-2.51 (m, 1H), 2.26-2.19 (m, 2H), 1.87-1.76 (m, 3H) ppm.

### 5-(4-nitro-1-(tetrahydro-2H-pyran-2-yl)-1H-indazol-6-yl)pyridazine-3-carbonitrile 2.316

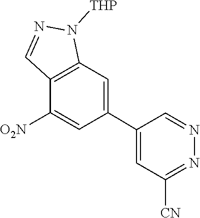

To a mixture of compound 2.315 (313 mg, 870.01 μmol) in DMA (6 mL) was added DPPF (96.46 mg, 174.00 μmol), Zn(CN)₂ (153.25 mg, 1.31 mmol) and Pd₂(dba)₃ (79.67 mg, 87.00 μmol). The mixture was degassed and purged with N₂ (×3), the mixture was stirred at 120° C. for 2 h. The reaction mixture was cooled to 20° C., diluted with DMF (6 mL) and filtered. The filtrate was poured onto H₂O (50 mL) and filtered. The filter cake was dried under vacuum to afford compound 2.316 (190 mg, 293.14 μmol, 33.69% yield) as a black-brown solid.

LCMS (AM12): rt=0.522 min, (351.2 [M+H]⁺), 54.05% purity.

### 5-(4-amino-1-(tetrahydro-2H-pyran-2-yl)-1H-indazol-6-yl)pyridazine-3-carbonitrile 2.317

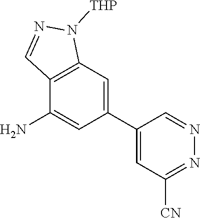

To a solution of compound 2.316 (190 mg, 542.34 μmol) in MeOH (5 mL) and H₂O (2 mL) was added NH₄Cl (232.09 mg, 4.34 mmol) and Fe (181.72 mg, 3.25 mmol). The mixture was stirred at 60° C. for 1 h. The reaction mixture was diluted with MeOH (30 mL) and filtered. The filtrate was concentrated in vacuo to give a residue, which was purified (PM52) to afford compound 2.317 (35 mg, 106.55 μmol, 19.65% yield) as a yellow solid.

LCMS (AM11): rt=0.398 min, (320.9 [M+H]⁺), 97.52% purity.

### Synthesis of Intermediate 2.320

### tert-butyl (3-chloro-4-(trifluoromethoxy)benzyl)(3-((3-((7-fluoro-6-(pyridin-4-yl)-1-(tetrahydro-2H-pyran-2-yl)-1H-indazol-4-yl)amino)propyl)amino)-3-oxopropyl)carbamate 2.320

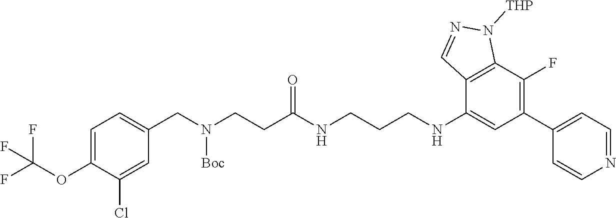

To a solution of compound 2.263 (80 mg, 256.13 μmol) in MeOH (1 mL) was added compound 1.950 (115.99 mg, 256.13 μmol) at 20° C. The mixture was stirred at 20° C. for 12 h, then NaBH₃CN (56.33 mg, 896.46 μmol) was added in portions at 0° C. The mixture was stirred at 20° C. for 1 h. The reaction mixture was purified (PM171) to give a solution, then the solution was basified to pH=9 by saturated NaHCO₃ solution and extracted with EA (30 mL×3). The combined organic phases were washed by brine (20 mL×2), dried (Na₂SO₄), filtered and concentrated in vacuo to afford compound 2.320 (70 mg, 70.22 μmol, 27.41% yield) as a yellow solid.

LCMS (AM11): rt=0.498 min, (749.30 [M+H]⁺), 75.15% purity.

### Synthesis of Intermediate 2.321

### 7-chloro-1-(tetrahydro-2H-pyran-2-yl)-6-(4H-1,2,4-triazol-4-yl)-1H-indazol-4-amine 2.321

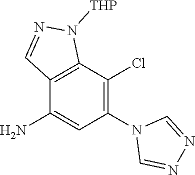

To a solution of compound 1.302 (500 mg, 1.76 mmol) in DMF (5 mL) was added NCS (234.83 mg, 1.76 mmol) at 0° C. The mixture was stirred at 0° C. for 0.5 h. The reaction mixture was poured into water (30 mL) and extracted with EA (30 mL×3). The combined organic phase was washed (brine, 30 mL×3), dried (Na₂SO₄), filtered and concentrated in vacuo to give a residue, then the residue was triturated with DMF (4 mL) and filtered. The filter cake was dried in vacuo to afford compound 2.321 (183 mg, 505.78 μmol, 28.76% yield) as a yellow solid.

LCMS (AM14): rt=0.430 min, (319.2 [M+H]⁺), 88.1% purity.

### Synthesis of Intermediate 2.326

### tert-butyl (3-chloro-4-(trifluoromethoxy)benzyl)(3-((3-((6-(6-cyano-2,3-dihydropyridazin-4-yl)-1-(tetrahydro-2H-pyran-2-yl)-1H-indazol-4-yl)amino)propyl)amino)-3-oxopropyl)carbamate 2.325

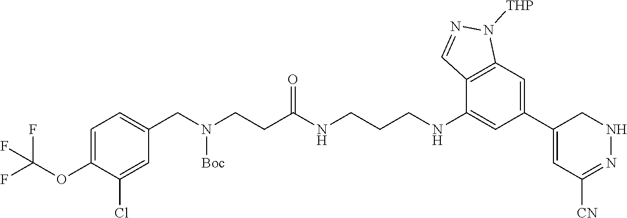

To a solution of compound 2.317 (70 mg, 175.95 μmol, 80.52% purity) in MeOH (1 mL) was added compound 1.950 (79.68 mg, 175.95 μmol) and AcOH (12.68 mg, 211.14 μmol). The mixture was stirred at 20° C. for 12 h, then NaBH₃CN (38.70 mg, 615.81 μmol) was added in portions at 0° C. The resulting mixture was stirred at 20° C. for 2 h. The mixture was filtered and the filtrate was purified (PM175) to afford compound 2.325 (48 mg, 60.50 μmol, 34.38% yield) as a yellow solid.

LCMS (AM11): rt=0.565 min, (781.3 [M+Na]⁺), 95.69% purity.

### tert-butyl (3-chloro-4-(trifluoromethoxy)benzyl)(3-((3-((6-(6-cyanopyridazin-4-yl)-1-(tetrahydro-2H-pyran-2-yl)-1H-indazol-4-yl)amino)propyl)amino)-3-oxopropyl)carbamate 2.326

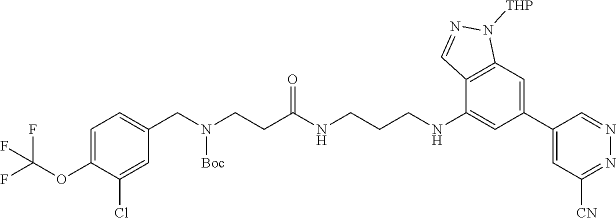

To a solution of compound 2.325 (24 mg, 31.61 μmol) in DCM (2 mL) was added MnO₂ (21.99 mg, 252.89 μmol), then the mixture was stirred at 20° C. for 2 h. The reaction mixture was filtered and the filtrate was concentrated in vacuo to afford compound 2.326 (36 mg, 43.80 μmol, 69.28% yield) as a yellow solid.

LCMS (AM11): rt=0.570 min, (757.5 [M+H]⁺), 92.13% purity.

### Synthesis of Intermediate 2.335

### tert-butyl (6-bromo-1-(tetrahydro-2H-pyran-2-yl)-1H-indazol-4-yl)carbamate 2.330

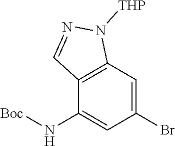

To a solution of compound 1.381 (1.25 g, 4.22 mmol) in THF (100 mL) was added Boc₂O (2.76 g, 12.66 mmol) dropwise, then the mixture was heated to 70° C. and stirred for 12 h, then Boc₂O (3.96 g, 18.15 mmol) and TEA (896.88 mg, 8.86 mmol) were added to the mixture at 20° C. The resulting mixture was stirred 70° C. for 12 h. The reaction mixture was poured into water (100 mL) and extracted with EA (100 mL×3). The combined organic phase was washed (brine, 100 mL), dried (Na₂SO₄), filtered and concentrated in vacuo to give a residue, which was purified (PM35) to afford compound 2.330 (1.25 g, 3.09 mmol, 73.24% yield) as a brown oil.

LCMS (AM16): rt=0.857 min, (398.1 [M+2+H]⁺), 73.6% purity.

### methyl 4-((tert-butoxycarbonyl)amino)-1-(tetrahydro-2H-pyran-2-yl)-1H-indazole-6-carboxylate 2.331

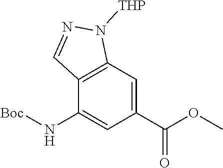

To a solution of compound 2.330 (1.25 g, 3.15 mmol) in MeOH (20 mL) was added Pd(dppf)Cl₂ (230.81 mg, 315.44 μmol) and TEA (957.56 mg, 9.46 mmol) under N₂. The suspension was degassed and purged with CO (×3). The mixture was stirred at 70° C. for 16 h under CO (50 psi). The mixture was poured into water (50 mL) and extracted with EA (50 mL×3). The combined organic phase was washed (brine, 50 mL), dried with anhydrous sodium sulfate, filtered and concentrated in vacuo to give a residue, which was purified (PM27) to afford compound 2.331 (1.0 g, 2.66 mmol, 84.45% yield) as a yellow oil.

¹H NMR (400 MHz, CHCl₃-d) δ 8.22 (s, 1H), 8.08 (s, 1H), 8.04 (s, 1H), 6.85 (s, 1H), 5.77 (dd, J=9.6 Hz, 2.4 Hz, 1H), 4.05 (d, J=11.2 Hz, 1H), 3.96 (s, 3H), 3.82-3.76 (m, 1H), 2.61-2.53 (m, 1H), 2.24-2.08 (m, 2H), 1.85-1.72 (m, 3H), 1.58 (s, 9H) ppm.

### tert-butyl (6-carbamoyl-1-(tetrahydro-2H-pyran-2-yl)-1H-indazol-4-yl)carbamate 2.332

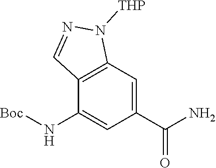

A solution of compound 2.331 (1 g, 2.66 mmol) in NH₃/MeOH (7 M, 50 mL) was stirred at 80° C. for 48 h in a 100 mL of sealed tube. The mixture was concentrated in vacuo to give a residue, which was purified (PM24) to afford compound 2.332 (892 mg, 2.43 mmol, 91.40% yield) as a yellow solid.

LCMS (AM12): rt=0.464 min, (221.0 [M-THP+2+H]⁺), 98.37% purity.

¹H NMR (400 MHz, CHCl₃-d) δ 8.06 (s, 1H), 8.03 (s, 1H), 7.93 (s, 1H), 6.91 (s, 1H), 5.76 (dd, J=9.6 Hz, 2.4 Hz, 1H), 4.06 (d, J=10.2 Hz, 1H), 3.82-3.75 (m, 1H), 2.59-2.50 (m, 1H), 2.19-2.06 (m, 2H), 1.84-1.71 (m, 3H), 1.58 (s, 9H) ppm.

### tert-butyl (6-(((dimethylamino)methylene)carbamoyl)-1-(tetrahydro-2H-pyran-2-yl)-1H-indazol-4-yl)carbamate 2.333

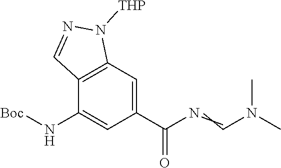

To a solution of compound 2.332 (800 mg, 2.22 mmol) in toluene (10 mL) was added DMF-DMA (793.52 mg, 6.66 mmol). The mixture was heated to 100° C. and stirred for 2 h. The mixture was concentrated in vacuo to afford compound 2.333 (1 g, crude) as a yellow oil.

¹H NMR (400 MHz, CHCl₃-d) δ 8.66 (s, 1H), 8.35 (s, 1H), 8.26 (s, 1H), 8.08 (s, 1H), 6.76 (s, 1H), 5.80 (dd, J=9.6 Hz, 2.4 Hz, 1H), 4.06 (d, J=10.8 Hz, 1H), 3.82-3.74 (m, 1H), 3.29 (s, 3H), 3.23 (s, 3H), 2.64-2.52 (m, 1H), 2.16 (s, 1H), 2.06 (d, J=12.0 Hz, 1H), 1.83-1.67 (m, 3H), 1.57 (s, 9H) ppm.

### tert-butyl (6-(1,2,4-oxadiazol-5-yl)-1-(tetrahydro-2H-pyran-2-yl)-1H-indazol-4-yl)carbamate 2.334

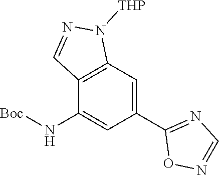

To a mixture of NH₂OH·HCl (200.70 mg, 2.89 mmol) and NaOH (5 M, 0.577 mL) in AcOH (10 mL) and dioxane (10 mL) was added compound 2.333 (1 g, 2.41 mmol) in one portion. The mixture was stirred at 25° C. for 30 min, then heated to 80° C. and stirred for 1 h. The mixture was poured into water (30 mL) and extracted with EA (30 mL×3). The combined organic phase was washed (brine, 30 mL), dried with anhydrous sodium sulfate, filtered and concentrated in vacuo to give a residue, which was purified (PM24) to afford compound 2.334 (489 mg, 1.27 mmol, 52.72% yield) as a yellow solid.

LCMS (AM12): rt=0.591 min, (386.2 [M+H]⁺), 74.7% purity.

### 6-(1,2,4-oxadiazol-5-yl)-1H-indazol-4-amine 2.335

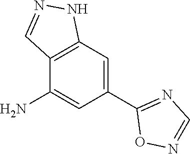 [SMILES: Nc1cc(-c2ncno2)cc2[nH]ncc12]

To a solution of compound 2.334 (489 mg, 1.27 mmol) in DCM (5 mL) was added TFA (451.82 mg, 3.96 mmol). The mixture was stirred at 20° C. for 1 h. The mixture was concentrated in vacuo to give a residue, which was diluted with ACN (10 mL) and basified to pH=8 by NH₃·H₂O. The mixture was filtered, the filter cake was dried in vacuo to afford compound 2.335 (152 mg, 755.53 μmol, 59.55% yield) as a yellow solid.

LCMS (AM16): rt=430 min, (202.2 [M+H]⁺), 98.5% purity.

### Synthesis of Intermediate 2.340

### 6-(1-ethoxyvinyl)-4-nitro-1-(tetrahydro-2H-pyran-2-yl)-1H-indazole 2.337

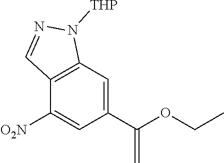

The mixture of 6-bromo-4-nitro-1-(tetrahydro-2H-pyran-2-yl)-1H-indazole (Ref: WO2019027960, 5 g, 15.33 mmol) in DMF (50 mL) was added tributyl(1-ethoxyvinyl)stannane (5.81 g, 16.10 mmol) and Pd(PPh₃)₄(1.77 g, 1.53 mmol). The mixture was heated to 100° C. and stirred for 2 h under N₂. The mixture was poured into saturated KF aqueous solution (100 mL) and stirred for 2 h. The mixture was filtered and the filtrate was extracted with EA (100 mL×3). The combined organic phase was washed (brine, 100 mL×3), dried with anhydrous sodium sulfate, filtered and concentrated in vacuo to give a residue, which was purified (PM27) to afford compound 2.337 (3.6 g, 11.34 mmol, 74.00% yield) as a yellow solid.

LCMS (AM16): rt=0.719 min, (318.2 [M+H]⁺), 68.6% purity.

### 2-bromo-1-(4-nitro-1-(tetrahydro-2H-pyran-2-yl)-1H-indazol-6-yl)ethan-1-one 2.338

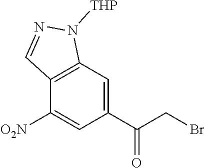

To a solution of compound 2.337 (3 g, 9.45 mmol) in THF (30 mL) and H₂O (6 mL) was added NBS (1.68 g, 9.45 mmol). The mixture was stirred at 25° C. for 1 h. The mixture was poured into water (50 mL) and extracted with EA (50 mL×3). The combined organic phase was washed (brine, 50 mL), dried with anhydrous sodium sulfate, filtered and concentrated in vacuo to afford compound 2.338 (3 g, crude) as a white solid.

¹H NMR (400 MHz, CHCl₃-d) δ 8.79-8.63 (m, 3H), 5.93 (dd, J=8.4 Hz, 2.4 Hz, 1H), 4.56 (s, 2H), 4.04-3.96 (m, 1H), 3.86-3.77 (m, 1H), 2.61-2.48 (m, 1H), 2.23-2.16 (m, 2H), 1.89-1.75 (m, 3H) ppm.

### 4-(4-nitro-1H-indazol-6-yl)oxazole 2.339

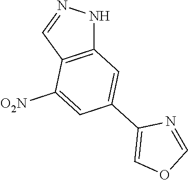 [SMILES: O=[N+]([O-])c1cc(-c2cocn2)cc2[nH]ncc12]

To a solution of compound 2.338 (2.4 g, 6.52 mmol) in formamide (67.80 g, 1.51 mol) was added H₂SO₄ (4.48 g, 45.63 mmol) in one portion, then the mixture was heated to 100° C. and stirred for 1 h. The mixture was poured into water (240 mL) and filtered. The filter cake was washed with ACN (15 mL), filtered and dried under vacuum to afford compound 2.339 (1.5 g, crude) as a brown solid.

LCMS (AM12): rt=0.406 min, (231.2 [M+H]⁺), 82.78% purity.

### 6-(oxazol-4-yl)-1H-indazol-4-amine 2.340

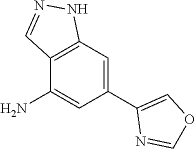 [SMILES: Nc1cc(-c2cocn2)cc2[nH]ncc12]

To a mixture of compound 2.339 (500 mg, 1.78 mmol, 82% purity) in MeOH (15 mL) and H₂O (5 mL) was added Fe (497.36 mg, 8.91 mmol) and NH₄Cl (476.40 mg, 8.91 mmol). The mixture was stirred at 60° C. for 2 h. The mixture was filtered, the filtrate was concentrated in vacuo to give a residue, which was purified (PM177) to afford compound 2.340 (100 mg, 439.57 μmol, 24.68% yield) as a grey solid.

LCMS (AM14): rt=0.325 min, (201.2 [M+H]⁺), 88.74% purity.

### Synthesis of Intermediate 2.347

### tert-butyl (4-(2-((6-bromo-1-(tetrahydro-2H-pyran-2-yl)-1H-benzo[d][1,2,3]triazol-4-yl)oxy)ethoxy)butyl)carbamate 2.344

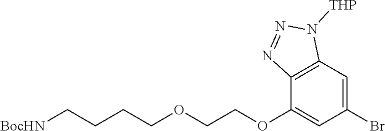

To a solution of tert-Butyl (4-(2-hydroxyethoxy)butyl)carbamate (CAS 2361304-25-6, 4.66 g, 19.99 mmol) in THF (200 mL) was added NaH (999.48 mg, 24.99 mmol, 60% purity). The reaction mixture was stirred at 0° C. for 1.5 h, then compound 2.041 (5 g, 16.66 mmol) was added. The reaction mixture was stirred at 60° C. for 12 h. The reaction mixture was diluted with H₂O (150 mL) and extracted with EA (100 mL×2). The combined organic phases were washed (brine, 150 mL×2), dried (Na₂SO₄), filtered and concentrated in vacuo to give a residue, which was purified (PM54) to afford compound 2.344 (4.5 g, 8.76 mmol, 52.61% yield) as a yellow oil.

LCMS (AM3): rt=1.016 min, (513.2 [M+H⁺]), 79.57% purity

### tert-butyl (4-(2-((6-amino-1-(tetrahydro-2H-pyran-2-yl)-1H-benzo[d][1,2,3]triazol-4-yl)oxy)ethoxy)butyl)carbamate 2.345

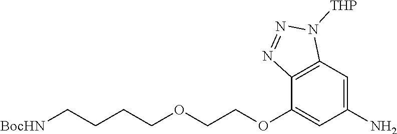

To a solution of compound 2.344 (900 mg, 1.75 mmol) in DMSO (8 mL) was added NH₃·H₂O (1.02 g, 7.30 mmol, 1.13 mL, 25% purity), K₂CO₃ (726.80 mg, 5.26 mmol), CuI (70.11 mg, 368.12 μmol) and (2S,4S)-4-hydroxypyrrolidine-2-carboxylic acid (94.24 mg, 718.70 μmol). The reaction mixture was stirred at 90° C. for 12 h. The reaction mixture was diluted with H₂O (20 mL), then extracted with EA (15 mL×2). The combined organic phases were washed (brine, 30 mL), dried (Na₂SO₄), filtered and concentrated in vacuo to afford compound 2.345 (800 mg, crude) as a yellow oil.

LCMS (AM3): rt=0.857 min, (450.3 [M+H⁺]), 95.77% purity.

### tert-butyl (4-(2-((1-(tetrahydro-2H-pyran-2-yl)-6-(4H-1,2,4-triazol-4-yl)-1H-benzo[d][1,2,3]triazol-4-yl)oxy)ethoxy)butyl)carbamate 2.346

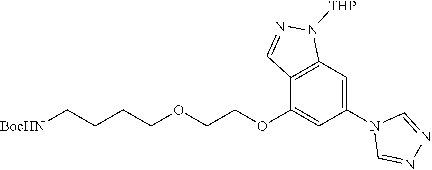

To a solution of compound 2.345 (700 mg, 1.56 mmol) in pyridine (20 mL) was added N-formamidoformamide (685.65 mg, 7.79 mmol), TMSCl (2.54 g, 23.36 mmol, 2.96 mL) and Et₃N (1.10 g, 10.90 mmol, 1.52 mL). The reaction mixture was stirred at 120° C. for 12 h. The reaction mixture was diluted with H₂O (150 mL) and extracted with EA (60 mL×2). The organic layers were washed (brine, 40 mL×2), dried (Na₂SO₄), filtered and concentrated in vacuo to give a residue, which was purified (PM23) to afford compound 2.346 (300 mg, 523.35 μmol, 33.61% yield) as a yellow oil.

LCMS (AM3): rt=0.899 min, (502.0 [M+H⁺]), 89.27% purity.

### 4-(2-((6-(4H-1,2,4-triazol-4-yl)-1H-benzo[d][1,2,3]triazol-4-yl)oxy)ethoxy)butan-1-amine 2.347

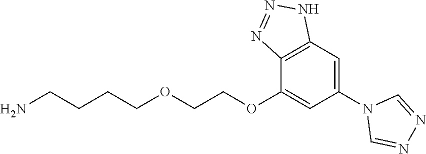 [SMILES: NCCCCOCCOc1cc(-n2cnnc2)cc2[nH]nnc12]

To a solution of compound 2.346 (250 mg, 498.43 μmol) in dioxane (3 mL) was added HCl/dioxane (4 M, 3 mL). The reaction mixture was stirred at 20° C. for 12 h. The reaction mixture was concentrated in vacuo to afford compound 2.347 (200 mg, HCl salt, crude) as a yellow solid, which was used for the next step directly.

LCMS (AM3): rt=0.707 min, (318.1 [M+H]⁺), 98.32% purity

### Synthesis of Intermediate 2.352

### methyl (E)-3-(4-nitro-1-(tetrahydro-2H-pyran-2-yl)-1H-indazol-6-yl)acrylate 2.348

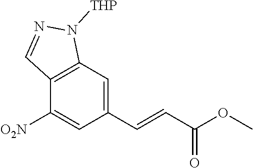

To a solution of 6-bromo-4-nitro-1-(tetrahydro-2H-pyran-2-yl)-1H-indazole (WO2019027960, 1 g, 3.07 mmol), methyl prop-2-enoate (527.92 mg, 6.13 mmol) and PPh₃ (804.20 mg, 3.07 mmol) in DMF (1 mL) was added TEA (620.51 mg, 6.13 mmol) and Pd(OAc)₂ (13.77 mg, 61.32 μmol) at 110° C. The mixture was stirred at 110° C. for 6 h. The reaction mixture was diluted with water 200 mL and extracted with EA (100 mL×3). The combined organic layers were washed (brine, 100 mL×2), dried (Na₂SO₄), filtered and concentrated in vacuo to give a residue, which was purified (PM27) to afford compound 2.348 (500 mg, 1.51 mmol, 49.22% yield) as a yellow solid.

### methyl 3-(4-amino-1-(tetrahydro-2H-pyran-2-yl)-1H-indazol-6-yl)propanoate 2.349

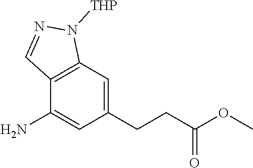

To a solution of compound 2.348 (500 mg, 1.51 mmol) in EA (5 mL) was added Pd/C (200 mg) at 20° C. The mixture was stirred under a H₂ atmosphere at 20° C. for 2 h. The catalyst was filtered and the filtrate was concentrated in vacuo to afford compound 2.349 (450 mg, crude), which was used for the next step without further purification.

### methyl 3-(4-(2-(4-((tert-butoxycarbonyl)amino)butoxy)acetamido)-1-(tetrahydro-2H-pyran-2-yl)-1H-indazol-6-yl)propanoate 2.350

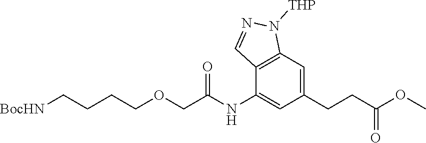

To a solution of compound 2.349 (450 mg, 1.48 mmol) and compound 1.891 (366.83 mg, 1.48 mmol) in DMF (5 mL) was added HATU (846.05 mg, 2.23 mmol) and DIPEA (383.44 mg, 2.97 mmol) at 20° C. The mixture was stirred at 20° C. for 10 min. The reaction mixture was diluted with water (200 mL) and extracted with EA (100 mL×3). The combined organic layers were washed (brine, 100 mL×2), dried (Na₂SO₄), filtered and concentrated in vacuo to give a residue, which was purified (PM88) to afford compound 2.350 (300 mg, 535.08 μmol, 36.07% yield) as a yellow oil.

LCMS (AM3): rt=0.940 min, (533.4 [M+H]⁺), 94.00% purity.

### methyl 3-(4-((2-(4-((tert-butoxycarbonyl)amino)butoxy)ethyl)amino)-1-(tetrahydro-2H-pyran-2-yl)-1H-indazol-6-yl)propanoate 2.351

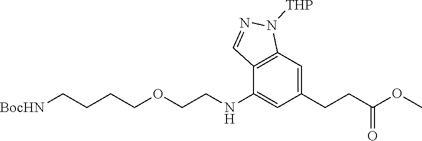

To a solution of compound 2.350 (300 mg, 563.25 μmol) in THF (2 mL) was added BH₃·THF (1 M, 1.13 mL) at 0° C. The mixture was stirred at 20° C. for 6 h. MeOH (20 mL) was added and the mixture was stirred at 20° C. for 1 h. The mixture was concentrated in vacuo to give a residue, which was purified (PM184) to afford compound 2.351 (70 mg, 134.97 μmol, 23.96% yield) as a yellow oil.

LCMS (AM3): rt=0.967 min, (519.3 [M+H]⁺), 98.8% purity.

### methyl 3-(4-((2-(4-aminobutoxy)ethyl)amino)-1H-indazol-6-yl)propanoate 2.352

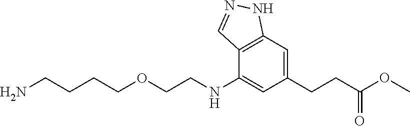 [SMILES: COC(=O)CCc1cc(NCCOCCCCN)c2cn[nH]c2c1]

To a solution of compound 2.351 (100 mg, 192.81 μmol) in MeOH (1 mL) was added HCl/MeOH (4 M, 513.48 μL) at 20° C. The mixture was stirred at 20° C. for 30 min. The residue was concentrated to afford compound 2.352 (60 mg, crude), which was used for the next step without further purification.

LCMS (AM3): rt=0.670 min, (335.3 [M+H]⁺), 73.83% purity.

### Synthesis of Intermediate 2.356

### tert-butyl (4-(2-((1H-indazol-4-yl)amino)-2-oxoethoxy)butyl)carbamate 2.354

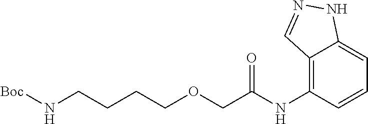 [SMILES: CC(C)(C)OC(=O)NCCCCOCC(=O)Nc1cccc2[nH]ncc12]

To a solution of 1H-indazol-4-amine (CAS 41748-71-4, 3 g, 22.53 mmol) and compound 1.891 (6.13 g, 24.78 mmol) in DMF (22 mL) was added HATU (12.85 g, 33.80 mmol) and DIPEA (5.82 g, 45.06 mmol, 7.85 mL). The reaction was stirred at 20° C. for 1 h. The reaction mixture was diluted with water (50 mL) and then extracted with EA (100 mL×3). The combined organic layers were washed (brine, 100 mL×2), dried (Na₂SO₄), filtered and concentrated in vacuo to give a residue, which was purified (PM35) to afford compound 2.354 (3 g, 8.28 mmol, 36.74% yield) as a yellow oil

LCMS (AM3): rt=0.855 min, (363.2 [M+H]⁺), 92.29% purity.

### tert-butyl (4-(2-((1H-indazol-4-yl)amino)ethoxy)butyl)carbamate 2.355

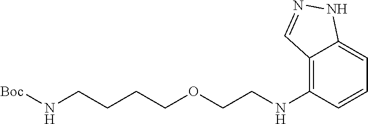 [SMILES: CC(C)(C)OC(=O)NCCCCOCCNc1cccc2[nH]ncc12]

To a solution of compound 2.354 (3 g, 8.28 mmol) in THF (30 mL) was added BH₃-DMS (628.84 mg, 8.28 mmol) at 0° C. The mixture was stirred at 2° C. for 12 h. The reaction mixture was diluted with water (100 mL) and extracted with EA (100 mL×3). The combined organic layers were washed (brine, 100 mL×2), dried (Na₂SO₄), filtered and concentrated in vacuo to afford compound 2.355 (3 g, crude) as a yellow solid.

LCMS (AM3): rt=0.743 min, (349.3 [M+H]⁺), 94.07% purity.

### N-(2-(4-aminobutoxy)ethyl)-1H-indazol-4-amine 2.356

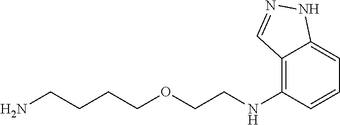 [SMILES: NCCCCOCCNc1cccc2[nH]ncc12]

A mixture of compound 2.355 (3 g, 8.61 mmol) in HCl/MeOH (4 M, 2.15 mL) was stirred at 20° C. for 2 h. The reaction mixture was concentrated in vacuo to give a residue, which was purified (PM185) to afford compound 2.356 (2 g, 7.02 mmol, 81.57% yield, HCl salt) as a yellow solid.

LCMS (AM3): rt=0.224 min, (249.3 [M+H]⁺), 80.87% purity.

### Synthesis of Intermediate 2.357

### tert-butyl (3-((3-((1H-indazol-4-yl)amino)propyl)amino)-3-oxopropyl)((2-chloro-[1,1′-biphenyl]-4-yl)methyl)carbamate 2.357

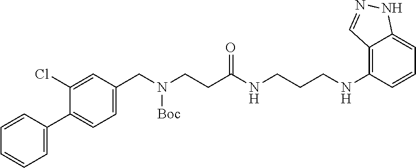 [SMILES: CC(C)(C)OC(=O)N(CCC(=O)NCCCNc1cccc2[nH]ncc12)Cc1ccc(-c2ccccc2)c(Cl)c1]

To a mixture of intermediate H (334.17 mg, 0.751 mmol) and 1H-indazol-4-amine (100 mg, 0.751 mmol) in DCE (10 mL) was added NaBH(OAc)₃ (318.35 mg, 1.50 mmol). The mixture was stirred at 25° C. for 12 h under N₂. The mixture was concentrated in vacuo to give a residue, which was purified (PM186) to afford compound 2.357 (100 mg, 0.178 mmol, 23.69% yield) as a white solid.

LCMS (AM3): rt=0.941 min, (562.3 [M+H⁺]), 99.53% purity.

### Synthesis of Intermediate 2.360

### methyl 4-amino-1-(tetrahydro-2H-pyran-2-yl)-1H-indazole-6-carboxylate 2.358

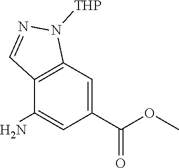

A mixture of compound 2.103 (0.5 g, 1.47 mmol), NH₃·H₂O (546.00 mg, 3.89 mmol, 0.6 mL, 25% purity), CuI (58.96 mg, 309.57 μmol), (2S,4S)-4-hydroxypyrrolidine-2-carboxylic acid (79.25 mg, 604.39 μmol) and K₂CO₃ (629.54 mg, 4.56 mmol) in DMSO (10 mL) was stirred at 90° C. for 16 h under N₂. The mixture was diluted with water (20 mL) and extracted with EA (20 mL×3). The combined organic phase was washed (brine, 40 mL×2), dried (Na₂SO₄), filtered and concentrated in vacuo to afford compound 2.358 (150 mg, 544.86 μmol, 36.96% yield) as a yellow oil, which was used directly for the next step.

LCMS (AM3): rt=0.777 min, (276.1 [M+H⁺]), 37.4% purity.

### methyl 4-((3-(3-((tert-butoxycarbonyl)((2-chloro-[1,1′-biphenyl]-4-yl)methyl)amino)propanamido)propyl)amino)-1-(tetrahydro-2H-pyran-2-yl)-1H-indazole-6-carboxylate 2.359

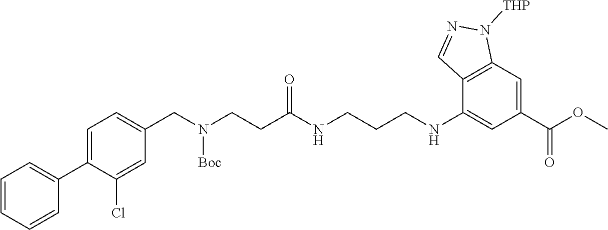

To a mixture of Intermediate H (242.43 mg, 544.86 μmol) and compound 2.358 (150 mg, 544.86 μmol) in DCE (10 mL) was added NaBH(OAc)₃ (230.95 mg, 1.09 mmol). The mixture was stirred at 25° C. for 16 h. The mixture was concentrated in vacuo to afford a residue, which was purified (PM187) to afford compound 2.359 (65 mg, 92.30 μmol, 16.94% yield) as a white solid.

LCMS (AM3): rt=1.107 min, (704.4 [M+H⁺]), 32.44% purity.

### tert-butyl ((2-chloro-[1,1′-biphenyl]-4-yl)methyl)(3-((3-((6-(hydroxymethyl)-1-(tetrahydro-2H-pyran-2-yl)-1H-indazol-4-yl)amino)propyl)amino)-3-oxopropyl)carbamate 2.360

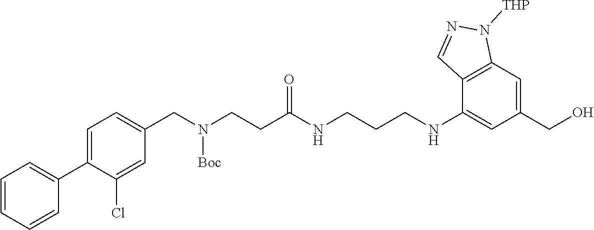

LiAlH₄ (5.39 mg, 141.99 μmol) was added to THF (10 mL) at 0° C., then compound 2.359 (50 mg, 71.00 μmol) in THF (2 mL) was added. The mixture was stirred at 25° C. for 16 h. The mixture was quenched with three drops of water and then Na₂SO₄ was added, then the mixture was filtered and the filtrate was concentrated in vacuo to afford compound 2.360 (48 mg, crude) as a yellow oil which was used directly into the next step.

LCMS (AM3): rt=0.966 min, (676.4 [M+H⁺]), 75.17% purity.

### Synthesis of Intermediate 2.362

### 4-((3-(3-((tert-butoxycarbonyl)((2-chloro-[1,1′-biphenyl]-4-yl)methyl)amino)propanamido)propyl)amino)-1-(tetrahydro-2H-pyran-2-yl)-1H-indazole-6-carboxylic acid 2.361

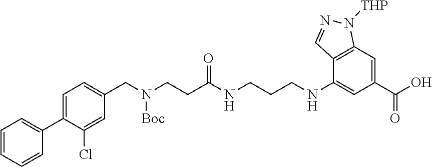

To a mixture of compound 2.359 (360 mg, 511.18 μmol) in THF (18 mL), MeOH (0.2 mL) and H₂O (0.2 mL) was added LiOH·H₂O (214.51 mg, 5.11 mmol). The mixture was stirred at 25° C. for 20 h. The mixture was concentrated in vacuo to give a residue, which was purified (PM184) to afford compound 2.361 (340 mg, 492.59 μmol, 96.36% yield) as a yellow solid.

LCMS (AM3): rt=0987 min, (690.4 [M+H⁺]), 86.73% purity.

### tert-butyl ((2-chloro-[1,1′-biphenyl]-4-yl)methyl)(3-((3-((6-(dimethylcarbamoyl)-1-(tetrahydro-2H-pyran-2-yl)-1H-indazol-4-yl)amino)propyl)amino)-3-oxopropyl)carbamate 2.362

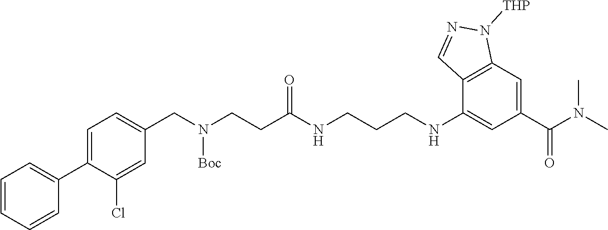

To a mixture of compound 2.361 (50 mg, 72.44 μmol) and N-methylmethanamine; hydrochloride (11.81 mg, 144.88 μmol) in THF (2 mL) was added HATU (33.05 mg, 86.93 μmol) and DIPEA (28.09 mg, 217.32 μmol). The mixture was stirred at 25° C. for 12 h. The mixture was diluted with water (10 mL) and extracted with EA (15 mL×2). The combined organic phase was washed (brine, 30 mL), dried (Na₂SO₄), filtered and concentrated in vacuo to afford compound 2.362 (52 mg, crude) as a yellow oil, which was used directly for the next step.

LCMS (AM3): rt=0.991 min, (717.4 [M+H⁺]), 84.8% purity.

### Synthesis of Intermediate 2.363

### tert-butyl(3-((3-((6-(4H-1,2,4-triazol-4-yl)-1H-indazol-4-yl)oxy)propyl)amino)propyl)(3-chloro-4-(trifluoromethoxy)benzyl)carbamate 2.363

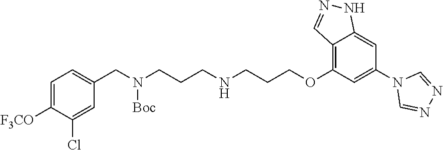 [SMILES: CC(C)(C)OC(=O)N(CCCNCCCOc1cc(-n2cnnc2)cc2[nH]ncc12)Cc1ccc(OC(F)(F)F)c(Cl)c1]

A mixture of compound 2.293 (110 mg, 288.13 μmol), compound 1.362 (100 mg, 339.28 μmol, HCl salt) and DIPEA (252.40 mg, 1.95 mmol) in MeOH (4 mL) was stirred at 20° C. for 12 h. NaBH₃CN (181.06 mg, 2.88 mmol) was added and the mixture was stirred at 20° C. for 16 h. The mixture was concentrated in vacuo to give a residue, which was purified (PM188) to afford compound 2.363 (40 mg, 64.10 μmol, 22.25% yield) as a yellow solid.

LCMS (AM3): rt=0.887 min, (624.2 [M+H]⁺), 100% purity.

### Synthesis of Intermediate 2.367

### tert-butyl(4-(2-((1-(tetrahydro-2H-pyran-2-yl)-6-(4,4,5,5-tetramethyl-1,3,2-dioxaborolan-2-yl)-1H-benzo[d][1,2,3]triazol-4-yl)oxy)ethoxy)butyl)carbamate 2.365

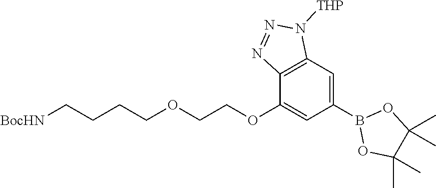

To a mixture of compound 2.344 (2 g, 3.90 mmol) and 4,4,5,5-tetramethyl-2-(4,4,5,5-tetramethyl-1,3,2-dioxaborolan-2-yl)-1,3,2-dioxaborolane (1.05 g, 4.13 mmol) in dioxane (20 mL) were added KOAc (764.61 mg, 7.79 mmol) and Pd(dppf)Cl₂ (285.03 mg, 389.54 μmol). The mixture was stirred at 85° C. for 12 h under N₂. The mixture was concentrated in vacuo to give a residue, which was purified (PM35) to afford compound 2.365 (1.69 g, 3.02 mmol, 77.40% yield) as a brown oil.

LCMS (AM3): rt=1.041 min, (561.8 [M+H]⁺), 57.78% purity.

### tert-butyl(4-(2-((6-(pyridazin-4-yl)-1-(tetrahydro-2H-pyran-2-yl)-1H-benzo[d][1,2,3]triazol-4-yl)oxy)ethoxy)butyl)carbamate 2.366

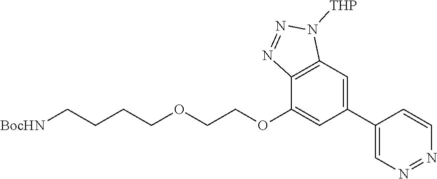

To a mixture of compound 2.365 (1.49 g, 2.66 mmol) and 4-bromopyridazine (701.51 mg, 2.92 mmol, HBr) in dioxane (15 mL) and H₂O (1.5 mL) were added Pd(dppf)Cl₂ (194.52 mg, 265.84 μmol) and K₂CO₃ (1.10 g, 7.98 mmol). The mixture was stirred at 80° C. for 12 h under N₂. The mixture was concentrated in vacuo to give a residue, which was purified (PM23) to afford compound 2.366 (1.1 g, 2.15 mmol, 80.72% yield) as a yellow oil.

LCMS (AM3): rt=0.878 min, (513.2 [M+H]⁺), 60.88% purity.

### 4-(2-((6-(pyridazin-4-yl)-1H-benzo[d][1,2,3]triazol-4-yl)oxy)ethoxy)butan-1-amine 2.367

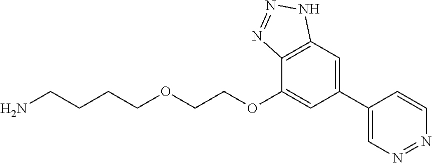 [SMILES: NCCCCOCCOc1cc(-c2ccnnc2)cc2[nH]nnc12]

To a mixture of compound 2.366 (1 g, 1.95 mmol) in MeOH (8 mL) was added HCl/dioxane (4 M). The mixture was stirred at 25° C. for 2 h. The mixture was concentrated in vacuo to afford compound 2.367 (750 mg, crude, HCl salt) as a yellow solid, which was used directly for the next step.

LCMS (AM3): rt=0.762 min, (329.1 [M+H]⁺), 98% purity.

### Synthesis of Intermediate 2.373

### 4-nitro-1H-indazole-6-carboxamide 2.369

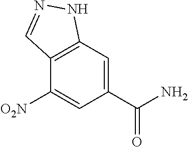 [SMILES: NC(=O)c1cc([N+](=O)[O-])c2cn[nH]c2c1]

To a 100 mL autoclave was placed a solution of methyl 4-nitro-1H-indazole-6-carboxylate (CAS 72922-61-3, 2 g, 9.04 mmol) in NH₃/MeOH (7 M, 50 mL). The mixture was stirred at 70° C. for 16 h. The reaction mixture was cooled to 20° C. and concentrated in vacuo to afford compound 2.369 (1.6 g, 7.76 mmol, 85.83% yield) as a yellow solid.

¹H NMR (400 MHz, DMSO-d₆) δ 14.17 (br s, 1H), 8.85-8.52 (m, 3H), 8.47 (s, 1H), 7.76 (s, 1H) ppm.

### 4-nitro-1H-indazole-6-carbothioamide 2.370

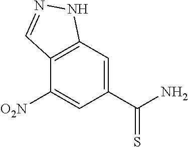 [SMILES: NC(=S)c1cc([N+](=O)[O-])c2cn[nH]c2c1]

To a mixture of compound 2.369 (1.5 g, 7.28 mmol) in THF (50 mL) was added Lawesson's reagent (2.35 g, 5.82 mmol). The mixture was heated to 60° C. and stirred for 2 h. The mixture was poured into water (150 mL). The suspension was filtered, the filter cake was collected and triturated with DCM (50 mL×2) at 20° C. for 10 min. The mixture was filtered and the filter cake was washed with MeOH (5 mL) and dried in vacuo to afford compound 2.370 (1 g, 4.31 mmol, 59.23% yield) as a yellow solid.

¹H NMR (400 MHz, DMSO-d₆) δ 14.22 (s, 1H), 10.25 (s, 1H), 9.96 (s, 1H), 8.74 (s, 1H), 8.58 (s, 1H), 8.54 (s, 1H) ppm.

### N-((dimethylamino)methylene)-4-nitro-1H-indazole-6-carbothioamide 2.371

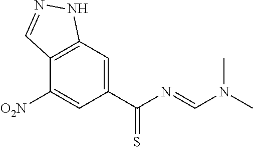 [SMILES: CN(C)/C=N/C(=S)c1cc([N+](=O)[O-])c2cn[nH]c2c1]

To a solution of compound 2.370 (1 g, 4.50 mmol) in dioxane (40 mL) was added DMF-DMA (1.61 g, 13.50 mmol). The mixture was heated to 80° C. and stirred for 2 h. The mixture was concentrated in vacuo to afford compound 2.371 (1.2 g, crude) as a yellow oil.

LCMS (AM11): rt=0.400 min, (277.9 [M+H]⁺), 88.15% purity.

### 5-(4-nitro-1H-indazol-6-yl)-1,2,4-thiadiazole compound 2.372

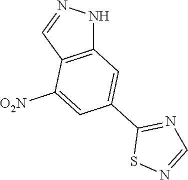 [SMILES: O=[N+]([O-])c1cc(-c2ncns2)cc2[nH]ncc12]

To a solution of compound 2.371 (1.2 g, 4.33 mmol) in EtOH (20 mL) was added O-(2,4-dinitrophenyl)hydroxylamine (861.68 mg, 4.33 mmol) and pyridine (342.30 mg, 4.33 mmol). The mixture was heated to 60° C. and stirred for 1 h. The mixture was concentrated in vacuo to give a residue, which was purified (PM189) to give a crude product that was triturated with ACN (2 mL) at 20° C. for 5 min and filtered. The filter cake was collected and dried in vacuo to afford compound 2.372 (200 mg, 716.73 μmol, 16.56% yield) as a yellow solid.

LCMS (AM11): rt=0.385 min, (248.0 [M+H]⁺), 88.69% purity.

### 6-(1,2,4-thiadiazol-5-yl)-1H-indazol-4-amine compound 2.373

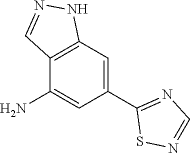 [SMILES: Nc1cc(-c2ncns2)cc2[nH]ncc12]

To a mixture of compound 2.372 (200 mg, 808.95 μmol) in H₂O (5 mL) and EtOH (5 mL) was added Fe (316.23 mg, 5.66 mmol) and NH₄Cl (346.18 mg, 6.47 mmol). The mixture was stirred at 50° C. for 1 h. The mixture was diluted with MeOH (10 mL), filtered and washed with MeOH (2 mL×2). The filtrate was concentrated in vacuo to afford compound 2.373 (140 mg, 644.42 μmol, 79.66% yield) as a yellow solid.

¹H NMR (400 MHz, DMSO-d₆) δ 13.04 (s, 1H), 8.91 (s, 1H), 8.20 (s, 1H), 7.33 (s, 1H), 6.76 (s, 1H), 6.20 (s, 2H) ppm.

### Synthesis of Intermediate 2.388

### 4-nitro-1-(tetrahydro-2H-pyran-2-yl)-6-vinyl-1H-indazole 2.384

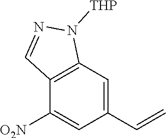

To a solution of 6-bromo-4-nitro-1-(tetrahydro-2H-pyran-2-yl)-1H-indazole (Ref: WO 2019027960, 1 g, 3.07 mmol) in dioxane (10 mL) and H₂O (1 mL) was added 4,4,5,5-tetramethyl-2-vinyl-1,3,2-dioxaborolane (519.45 mg, 3.37 mmol), K₂CO₃ (881.44 mg, 6.38 mmol) and Pd(dppf)Cl₂ (226.70 mg, 309.83 μmol). The reaction mixture was degassed and purged with N₂ (×3) then stirred at 80° C. for 12 h. The reaction mixture was filtered and the filtrate was concentrated in vacuo to give a residue, which was purified (PM7) to afford compound 2.384 (700 mg, 2.56 mmol, 83.54% yield) as a yellow solid.

¹H NMR (400 MHz, CHCl₃-d) δ 8.59 (s, 1H), 8.29 (s, 1H), 7.89 (s, 1H), 6.89 (dd, J=17.6, 11.2 Hz, 1H), 5.99 (d, J=17.2 Hz, 1H), 5.84-5.80 (m, 1H), 5.51 (d, J=10.8 Hz, 1H), 4.02-3.97 (m, 1H), 3.82-3.76 (m, 1H), 2.58-2.53 (m, 1H), 2.20-2.15 (m, 2H), 1.81-1.71 (m, 3H) ppm

### 4-nitro-1-(tetrahydro-2H-pyran-2-yl)-1H-indazole-6-carbaldehyde 2.385

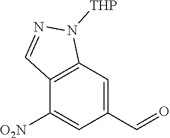

A solution of compound 2.384 (3.15 g, 11.53 mmol) in DCM (50 mL) was purged with ozone at −70° C. for 0.2 h until the mixture turned blue. The mixture was then purged by N₂ at −70° C. for 0.1 h until the mixture turned clear. DMS (12.69 g, 204.25 mmol, 15 mL) was added at 0° C. and the mixture was stirred at 20° C. for 12 h. The reaction mixture was concentrated in vacuo to give a residue, which was purified (PM36) to afford compound 2.385 (1.6 g, 5.81 mmol, 50.43% yield) as a yellow solid.

¹H NMR (400 MHz, CHCl₃-d) δ 10.14 (s, 1H), 8.64 (s, 1H), 8.56 (s, 1H), 8.47 (s, 1H), 5.87-5.83 (m, 1H), 3.95-3.91 (m, 1H), 3.79-3.72 (m, 1H), 2.47-2.41 (m, 1H), 2.14-2.11 (m, 2H), 1.72-1.67 (m, 3H) ppm

### 5-(4-nitro-1-(tetrahydro-2H-pyran-2-yl)-1H-indazol-6-yl)oxazole 2.386

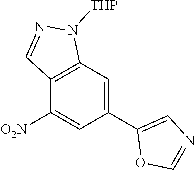

To a solution of compound 2.385 (1.48 g, 5.38 mmol) and 1-(isocyanomethylsulfonyl)-4-methyl-benzene (1 g, 5.12 mmol) in MeOH (20 mL) was added K₂CO₃ (1.06 g, 7.68 mmol). The mixture was stirred at 70° C. for 2 h. The reaction mixture was diluted with H₂O (100 mL) and extracted with EA (60 mL×2). The organic layer was washed (brine, 100 mL), dried (Na₂SO₄), filtered and concentrated in vacuo to give a residue, which was purified (PM36) to afford compound 2.386 (1.5 g, 4.77 mmol, 93.18% yield) as a yellow solid.

LCMS (AM3): rt=0.885 min, (315.0 [M+H]⁺), 91.37% purity

### 6-(oxazol-5-yl)-1-(tetrahydro-2H-pyran-2-yl)-1H-indazol-4-amine 2.387

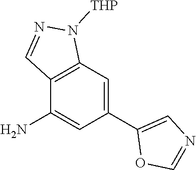

To a solution of compound 2.386 (1.45 g, 4.61 mmol) in EtOH (40 mL) and H₂O (8 mL) was added Fe (1.29 g, 23.07 mmol) and NH₄Cl (1.23 g, 23.07 mmol). The reaction mixture was stirred at 80° C. for 0.5 h. The mixture was filtered and the filtrate was concentrated in vacuo to give a residue. The residue was diluted with H₂O (100 mL) and extracted with EA (60 mL×2). The organic layer was washed (brine, 100 mL), dried (Na₂SO₄), filtered and concentrated in vacuo to give a residue, which was purified (PM25) to afford compound 2.387 (1.2 g, 4.20 mmol, 91.03% yield, 99.5% purity) as a yellow solid.

LCMS (AM3): rt=0.597 min, (284.9 [M+H]⁺), 99.58% purity

### 6-(oxazol-5-yl)-1H-indazol-4-amine 2.388

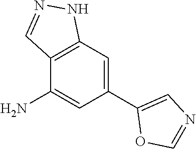 [SMILES: Nc1cc(-c2cnco2)cc2[nH]ncc12]

To a solution of compound 2.387 (650 mg, 2.29 mmol) in DCM (5 mL) was added TFA (7.70 g, 67.53 mmol, 5 mL). The mixture was stirred at 25° C. for 1 h. The mixture was concentrated in vacuo to give a residue, which was purified (PM191) to afford compound 2.388 (280 mg, 1.17 mmol, 51.33% yield) as a yellow solid.

### Synthesis of Intermediate 2.392

### 6-(3-methylpyridin-4-yl)-1-(tetrahydro-2H-pyran-2-yl)-1H-indazol-4-amine 2.391

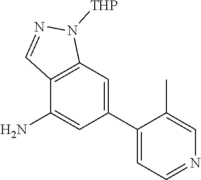

A mixture of compound 1.952 (1.5 g, 4.37 mmol), 4-chloro-3-methyl-pyridine (780.53 mg, 6.12 mmol), Pd(dppf)Cl₂ (319.78 mg, 437.03 μmol) and Cs₂CO₃ (4.27 g, 13.11 mmol) in dioxane (8 mL) and H₂O (1.6 mL) was degassed and purged with N₂ (×3), then the mixture was stirred at 100° C. for 12 h under N₂.The mixture was diluted with water (50 ml) and extracted with EA (60 mL×3). The combined organic phase was washed (brine, 55 mL), dried (Na₂SO₄), filtered and concentrated in vacuo to give a residue, which was purified (PM38) to afford compound 2.391 (756 mg, 2.40 mmol, 54.97% yield) as a yellow solid.

LCMS (AM3): rt=0.650 min, (309.0, [M+H]⁺), 98.1% purity.

### 6-(3-methylpyridin-4-yl)-1H-indazol-4-amine 2.392

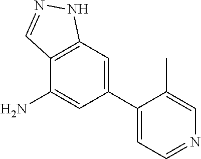 [SMILES: Cc1cnccc1-c1cc(N)c2cn[nH]c2c1]

To a solution of compound 2.391 (750 mg, 2.43 mmol) in MeOH (2 mL) was added HCl/dioxane (10 mL, 4 M). The mixture was stirred at 25° C. for 30 min. The reaction mixture was concentrated in vacuo to give a residue. The residue was dissolved in MeOH (10 mL), basified by NH₃·H₂O to pH=10 and filtered. The filtrate was purified (PM195) to afford compound 2.392 (0.4 g, 1.76 mmol, 72.24% yield) as an off-white solid.

LCMS (AM7): rt=0.721 min, (225.1, [M+H]⁺), 98.5% purity.

### Synthesis of Intermediate 2.396

### tert-butyl (4-(2-((5-chloro-1-(tetrahydro-2H-pyran-2-yl)-6-(4H-1,2,4-triazol-4-yl)-1H-indazol-4-yl)amino)ethoxy)butyl)(3,5-difluoro-4-(trifluoromethoxy)benzyl)carbamate 2.396

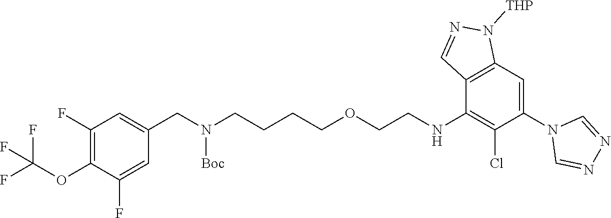

To a solution of compound 2.395 (188 mg, 264.90 μmol) in DMF (2 mL) was added NCS (38.91 mg, 291.39 μmol) at 0° C. The mixture was stirred at 0° C. for 0.5 h. The reaction mixture was poured into saturated Na₂SO₃ aqueous solution (30 mL) and extracted with EA (30 mL×3). The combined organic phase was washed (brine, 30 mL×3), dried (Na₂SO₄), filtered and concentrated in vacuo to give residue, which was purified by prep-TLC (EA) to afford compound 2.396 (79 mg, 105.95 μmol, 40.00% yield) as a colourless gum.

LCMS (AM14): rt=0.767 min, (744.3, [M+H]⁺), 99.89% purity.

### Synthesis of Intermediate 2.411

### tert-butyl (3-((2-((6-bromo-1-(tetrahydro-2H-pyran-2-yl)-1H-indazol-4-yl)oxy)ethoxy)methyl)cyclobutyl)carbamate 2.408

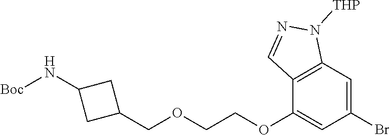

To a solution of compound 1.856 (1 g, 4.08 mmol) in DMF (10 mL) was added NaH (489.12 mg, 12.23 mmol, 60% purity). The mixture was stirred at 0° C. for 30 min, then a solution of 6-bromo-4-fluoro-1-(tetrahydro-2H-pyran-2-yl)-1H-indazole (1.22 g, 4.08 mmol) in DMF (10 mL) was added and the mixture was stirred at 0° C. for 1 h. The reaction mixture was dropwise added into saturated NH₄Cl aqueous solution (50 mL) at 0° C. and extracted with EA (40 mL×3). The combined organic phase was washed by brine (50 mL×4), dried (Na₂SO₄), filtered and concentrated in vacuo to give a residue, which was purified (PM24) to afford compound 2.408 (1.12 g, 2.14 mmol, 52.39% yield) as a light-yellow oil.

¹H NMR (400 MHz, CHCl₃-d) δ 8.04 (s, 1H), 7.37 (s, 1H), 6.67-6.62 (m, 1H), 5.61 (dd, J=9.6 Hz, 2.4 Hz, 1H), 4.72 (s, 1H), 4.27 (t, J=4.4 Hz, 2H), 4.08-3.98 (m, 2H), 3.89-3.84 (m, 2H), 3.78-3.70 (m, 1H), 3.50 (d, J=5.2 Hz, 2H), 2.55-2.40 (m, 3H), 2.27-2.16 (m, 2H), 2.16-2.10 (m, 1H), 1.80-1.72 (m, 2H), 1.69-1.62 (m, 3H), 1.45-1.40 (m, 9H) ppm.

### tert-butyl (3-((2-((1-(tetrahydro-2H-pyran-2-yl)-6-(4,4,5,5-tetramethyl-1,3,2-dioxaborolan-2-yl)-1H-indazol-4-yl)oxy)ethoxy)methyl)cyclobutyl)carbamate 2.409

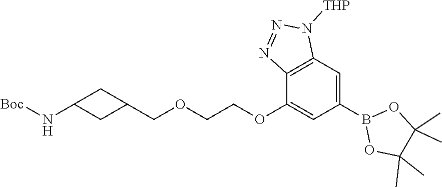

To a solution of compound 2.408 (1 g, 1.91 mmol) and 4,4,5,5-tetramethyl-2-(4,4,5,5-tetramethyl-1,3,2-dioxaborolan-2-yl)-1,3,2-dioxaborolane (581.04 mg, 2.29 mmol) in dioxane (15 mL) was added KOAc (561.41 mg, 5.72 mmol) and Pd(dppf)Cl₂ (139.52 mg, 190.68 μmol). The mixture was degassed and purged with N₂ (×3), then heated to 80° C. and stirred for 3 h. The reaction mixture was diluted with EA (50 mL) and filtered, the filtrate was concentrated in vacuo to give a residue, which was purified (PM28) to afford compound 2.409 (1 g, 1.75 mmol, 91.76% yield) as a yellow oil.

¹H NMR (400 MHz, CHCl₃-d) δ 8.12 (s, 1H), 7.63 (s, 1H), 6.91-6.88 (m, 1H), 5.78-5.72 (m, 1H), 4.71 (s, 1H), 4.34 (t, J=4.4 Hz, 2H), 4.10-4.03 (m, 2H), 3.92-3.85 (m, 2H), 3.81-3.74 (m, 1H), 3.54-3.49 (m, 2H), 2.66-2.55 (m, 1H), 2.48-2.40 (m, 2H), 2.27-2.14 (m, 2H), 2.01-1.94 (m, 1H), 1.84-1.71 (m, 2H), 1.60-1.57 (m, 3H), 1.44-1.40 (m, 9H), 1.38 (s, 12H) ppm.

### tert-butyl (3-((2-((6-(6-hydroxypyridazin-4-yl)-1-(tetrahydro-2H-pyran-2-yl)-1H-indazol-4-yl)oxy)ethoxy)methyl)cyclobutyl)carbamate 2.410

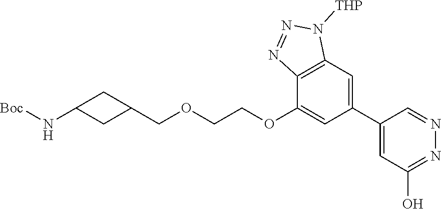

To a solution of compound 2.409 (600 mg, 1.05 mmol) and 4-chloro-1H-pyridazin-6-one (150.76 mg, 1.16 mmol) in dioxane (6 mL) and H₂O (1 mL) was added K₂CO₃ (290.19 mg, 2.10 mmol) and Pd(dppf)Cl₂ (76.82 mg, 105.00 μmol). The mixture was degassed and purged with N₂, then heated to 100° C. and stirred for 12 h. The reaction mixture was filtered and the filtrate was concentrated in vacuo to give a residue, which was purified (PM23) to afford compound 2.410 (320 mg, 593.01 μmol, 56.48% yield) as a light-yellow solid.

LCMS (AM11): rt=0.456 min, (540.4, [M+H]⁺), 71.89% purity.

¹H NMR (400 MHz, CHCl₃-d) δ 10.46 (s, 1H), 8.15 (s, 1H), 7.40 (s, 1H), 7.16-7.12 (m, 1H), 6.67-6.64 (m, 1H), 5.77-5.72 (m, 1H), 4.72 (s, 1H), 4.35 (t, J=4.4 Hz, 2H), 4.08-4.01 (m, 1H), 3.95-3.89 (m, 2H), 3.81-3.74 (m, 1H), 3.53 (d, J=5.6 Hz, 1H), 3.40 (t, J=2.8 Hz, 1H), 2.86 (s, 1H), 2.63-2.51 (m, 1H), 2.49-2.40 (m, 2H), 2.27-2.13 (m, 2H), 2.04-1.97 (m, 1H), 1.82-1.70 (m, 3H), 1.70-1.62 (m, 2H), 1.45-1.40 (m, 9H) ppm.

### 5-(4-(2-((3-aminocyclobutyl)methoxy)ethoxy)-1H-indazol-6-yl)pyridazin-3-ol 2.411

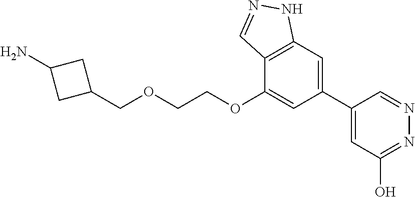 [SMILES: NC1CC(COCCOc2cc(-c3cnnc(O)c3)cc3[nH]ncc23)C1]

To a solution of compound 2.410 (300 mg, 555.94 μmol) in MeOH (6 mL) was added HCl/dioxane (4 M, 3 mL). The mixture was stirred at 20° C. for 0.5 h. The mixture was concentrated in vacuo to give a residue, which was triturated with MTBE (5 mL) at 20° C. for 10 min and filtered. The filter cake was dried in vacuum to afford compound 2.411 (227 mg, 529.99 μmol, 95.33% yield, HCl salt) as a yellow solid.

LCMS (AM11): rt=0.247 min, (356.4, [M+H]⁺), 89.41% purity.

### Synthesis of Intermediate 2.432

### tert-butyl (S)-(5-(2-oxoethoxy)pentan-2-yl)carbamate 2.430

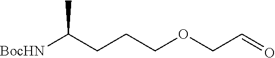 [SMILES: C[C@@H](CCCOCC=O)NC(=O)OC(C)(C)C]

To a solution of oxalyl dichloride (615.84 mg, 4.85 mmol) in DCM (6 mL) was added a solution of DMSO (568.61 mg, 7.28 mmol) in DCM (3 mL) dropwise at −70° C. under N₂. The mixture was stirred at −70° C. for 0.5 h, then a solution of compound 1.848 (600 mg, 2.43 mmol) in DCM (3 mL) was added dropwise at −70° C. and the resulting mixture was stirred at −70° C. for 0.5 h, then TEA (1.47 g, 14.56 mmol) was added dropwise at −70° C. and the mixture was stirred at −70° C. for 10 min. The mixture was filtered and the filtrate was concentrated in vacuo to give a residue, which was purified (PM29) to afford compound 2.430 (460 mg, 1.88 mmol, 77.30% yield) as a yellow oil.

¹H NMR (400 MHz, CHCl₃-d) δ 9.74 (s, 1H), 4.45-4.28 (m, 1H), 4.07 (s, 2H), 3.73-3.61 (m, 1H), 3.56 (t, J=6.4 Hz, 2H), 1.73-1.64 (m, 2H), 1.55-1.49 (m, 2H), 1.45 (s, 9H), 1.14 (d, J=6.4 Hz, 3H) ppm.

### tert-butyl ((2S)-5-(2-((1-(tetrahydro-2H-pyran-2-yl)-6-(4H-1,2,4-triazol-4-yl)-1H-indazol-4-yl)amino)ethoxy)pentan-2-yl)carbamate 2.431

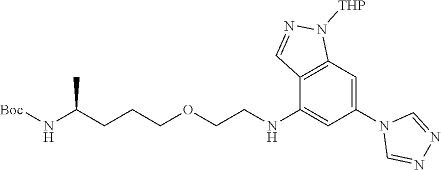

To a mixture of compound 2.430 (446.51 mg, 1.82 mmol) and compound 1.273 (450 mg, 1.58 mmol) in MeOH (6 mL) was added MgSO₄ (571.54 mg, 4.75 mmol) and HOAc (95.05 mg, 1.58 mmol). The mixture was stirred at 20° C. for 12 h, then NaBH₃CN (198.93 mg, 3.17 mmol) was added and the mixture was stirred at 20° C. for 1 h. The mixture was filtered and the filtrate was concentrated in vacuo to give a residue, which was purified (PM194) to afford compound 2.431 (420 mg, 783.36 μmol, 49.49% yield) as a yellow oil.

LCMS (AM11): rt=0.446 min, (514.4, [M+H]⁺), 95.80% purity.

¹H NMR (400 MHz, CHCl₃-d) δ 8.53 (s, 2H), 8.10 (s, 1H), 6.87 (s, 1H), 6.13 (s, 1H), 5.65 (dd, J=9.6, 2.4 Hz, 1H), 4.37-4.48 (m, 1H), 4.04 (br d, J=11.6 Hz, 1H), 3.80-3.68 (m, 4H), 3.55-3.45 (m, 4H), 2.59-2.47 (m, 1H), 2.21-2.07 (m, 2H), 1.72-1.63 (m, 5H), 1.50 (br s, 2H), 1.45 (s, 9H), 1.11 (d, J=6.8 Hz, 3H) ppm

### (S)-N-(2-((4-aminopentyl)oxy)ethyl)-6-(4H-1,2,4-triazol-4-yl)-1H-indazol-4-amine 2.432

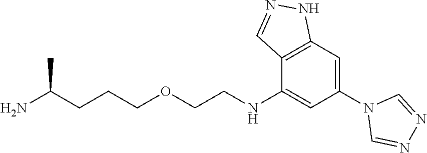 [SMILES: C[C@H](N)CCCOCCNc1cc(-n2cnnc2)cc2[nH]ncc12]

To a solution of compound 2.431 (370 mg, 720.36 μmol) in MeOH (8 mL) was added HCl/dioxane (4 M, 8 mL). The mixture was stirred at 20° C. for 0.5 h. The mixture was concentrated in vacuo to afford compound 2.432 (220 mg, crude, HCl salt) as a brown gum.

LCMS (AM11): rt=0.247 min, (330.0, [M+H]⁺), 93.47% purity.

### Synthesis of Intermediate 2.454

### 2-(4-nitro-1-(tetrahydro-2H-pyran-2-yl)-1H-indazol-6-yl)acetic acid 2.449

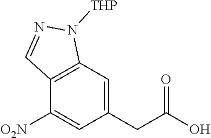

To a solution of compound 1.991 (4 g, 13.92 mmol) in H₂O (40 mL), ACN (20 mL) and DCM (20 mL) was slowly added RuCl₃·3H₂O (364.03 mg, 1.39 mmol) and NaIO₄ (11.91 g, 55.69 mmol,) at 0° C., then the mixture was warmed to 20° C. and stirred for 2 h. The mixture was poured into water (100 mL) and extracted with EA (80 mL×3). The aqueous phase was adjusted to pH=6 by 1 M HCl and extracted with EA (80 mL×3). The combined organic phase was dried (Na₂SO₄), filtered and concentrated in vacuo to afford compound 2.449 (3.3 g, 10.81 mmol, 77.64% yield) as an off-white solid.

LCMS (AM11): rt=0.369 min, (221.8 [M-THP]⁺), 88.51% purity.

¹H NMR (400 MHz, DMSO-d₆) δ 12.59 (s, 1H), 8.52 (s, 1H), 8.22 (s, 1H), 8.17 (s, 1H), 8.97 (dd, J=10.0 Hz, 2.4 Hz, 1H), 3.94 (s, 2H), 3.92-3.87 (m, 1H), 3.81-3.73 (m, 1H), 2.45-2.36 (m, 1H), 2.10-2.00 (m, 2H), 1.82-1.69 (m, 1H), 1.64-1.56 (m, 2H) ppm.

### 2-(4-nitro-1-(tetrahydro-2H-pyran-2-yl)-1H-indazol-6-yl)acetamide 2.450

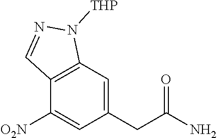

To a solution of compound 2.449 (3.3 g, 10.81 mmol) in THF (40 mL) was added CDI (2.28 g, 14.05 mmol). The mixture was stirred at 20° C. for 1 h, then NH₃·H₂O (5.41 g, 43.24 mmol, 28% purity) was added and the resulting mixture was stirred at 20° C. for 12 h. The reaction mixture was poured into water (50 mL) and the mixture was filtered. The filter cake was dried in vacuum to afford compound 2.450 (2.4 g, 7.89 mmol, 72.96% yield) as an off-white solid.

LCMS (AM11): rt=0.336 min, (220.8 [M-THP]⁺), 99.26% purity.

¹H NMR (400 MHz, DMSO-d₆) δ 8.52 (s, 1H), 8.15 (s, 2H), 7.63 (s, 1H), 7.06 (s, 1H), 6.01-5.96 (m, 1H), 3.94-3.86 (m, 1H), 3.82-3.74 (m, 1H), 3.71 (s, 2H), 2.45-2.36 (m, 1H), 2.10-2.00 (m, 2H), 1.83-1.70 (m, 1H), 1.65-1.57 (m, 2H) ppm.

### 2-(4-nitro-1-(tetrahydro-2H-pyran-2-yl)-1H-indazol-6-yl)acetonitrile 2.451

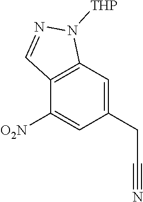

To a solution of compound 2.450 (2.4 g, 7.89 mmol) in pyridine (40 mL) was added TFAA (6 mL, 43.14 mmol) at 0° C., the mixture warmed to 20° C. and stirred for 0.5 h. The reaction was concentrated in vacuo to give a residue. The residue was resolved into water (80 mL), then extracted with EA (60 mL×2). The combined organic phase was washed (brine, 80 mL×3), dried (Na₂SO₄), filtered and concentrated in vacuo to give a residue, which was purified (PM27) to afford compound 2.451 (1.9 g, 6.64 mmol, 84.15% yield) as a yellow solid.

LCMS (AM11): rt=0.406 min, (202.8 [M-THP]⁺), 99.21% purity.

¹H NMR (400 MHz, CHCl₃-d) δ 8.63 (s, 1H), 8.09 (s, 1H), 8.04 (s, 1H), 5.84 (dd, J=8.8 Hz, 2.4 Hz, 1H), 4.02 (s, 1H), 4.01-3.96 (m, 1H), 3.84-3.76 (m, 1H), 2.60-2.48 (m, 1H), 2.24-2.12 (m, 2H), 1.88-1.79 (m, 1H), 1.79-1.68 (m, 2H) ppm.

### 2-(4-amino-1-(tetrahydro-2H-pyran-2-yl)-1H-indazol-6-yl)acetonitrile 2.452

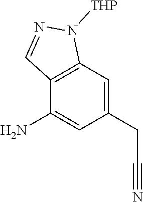

To a solution of compound 2.451 (650 mg, 2.27 mmol) in MeOH (12 mL) and H₂O (6 mL) was added NH₄Cl (971.60 mg, 18.16 mmol) and Fe (760.76 mg, 13.62 mmol), then the mixture was heated to 60° C. and stirred for 1 h. The reaction mixture was poured into water (30 mL) and extracted with EA (30 mL×3). The combined organic phase was washed (brine, 30 mL×3), dried (Na₂SO₄), filtered and concentrated in vacuo to afford compound 2.452 (583 mg, crude) as a yellow solid.

LCMS (AM11): rt=0.329 min, (257.4 [M+H]⁺), 99.36% purity.

¹H NMR (400 MHz, DMSO-d₆) δ 8.10 (s, 1H), 6.71 (s, 1H), 6.16 (s, 1H), 6.00 (s, 2H), 5.63 (dd, J=9.6 Hz, 2.4 Hz, 1H), 3.96 (s, 2H), 3.91-3.83 (m, 1H), 3.73-3.64 (m, 1H), 2.42-2.31 (m, 1H), 2.06-1.99 (m, 1H), 1.96-1.86 (m, 1H), 1.80-1.66 (m, 1H), 1.60-1.51 (m, 2H) ppm.

### tert-butyl (4-(2-((6-(cyanomethyl)-1-(tetrahydro-2H-pyran-2-yl)-1H-indazol-4-yl)amino)ethoxy)butyl)carbamate 2.453

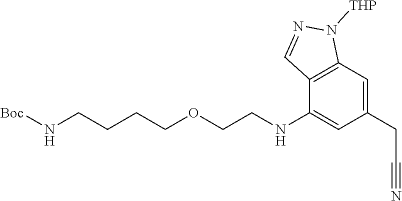

To a solution of compound 2.452 (200 mg, 780.33 μmol) and tert-Butyl (4-(2-aminoethoxy)butyl)carbamate (CAS 2830620-37-4, 0.53 mg, 858.36 μmol) in MeOH (5 mL) was added MgSO₄ (281.78 mg, 2.34 mmol) and AcOH (46.86 mg, 780.33 μmol). The mixture was stirred at 20° C. for 12 h, then NaBH₃CN (98.07 mg, 1.56 mmol) was added and the mixture was stirred at 20° C. for 1 h. Additional tert-Butyl (4-(2-aminoethoxy)butyl)carbamate (CAS 2830620-37-4, 30 mg, 129.78 μmol) was added, and the mixture was stirred at 20° C. for 12 h. The mixture was poured into water (20 mL) and extracted with EA (20 mL×3). The combined organic phase was washed (brine, 20 mL×3), dried (Na₂SO₄), filtered and concentrated in vacuo to give a residue, which was purified (PM196) to afford compound 2.453 (155 mg, 328.67 μmol, 42.12% yield) as a brown oil.

LCMS (AM11): rt=0.512 min, (472.2 [M+H]⁺), 34.59% purity.

¹H NMR (400 MHz, CHCl₃-d) δ 7.97 (s, 1H), 6.87 (s, 1H), 6.13 (s, 1H), 6.64 (dd, J=8.8 Hz, 2.4 Hz, 1H), 4.68 (s, 1H), 4.08-4.01 (s, 1H), 3.80 (s, 1H), 3.78-3.74 (m, 1H), 3.71 (t, J=4.8 Hz, 2H), 3.54-3.44 (m, 4H), 3.19-3.10 (m, 2H), 2.60-2.49 (m, 1H), 2.19-2.11 (m, 1H), 2.04-2.00 (m, 1H), 1.81-1.73 (m, 3H), 1.60-1.52 (m, 4H), 1.44 (s, 9H) ppm.

### 2-(4-((2-(4-aminobutoxy)ethyl)amino)-1H-indazol-6-yl)acetonitrile 2.454

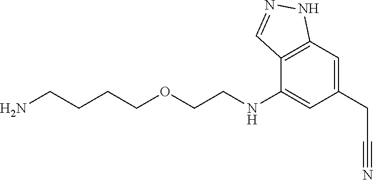 [SMILES: N#CCc1cc(NCCOCCCCN)c2cn[nH]c2c1]

To a solution of compound 2.453 (130 mg, 275.66 μmol) in DCM (10 mL) was added TFA (2 mL, 27.01 mmol), the mixture was stirred at 20° C. for 0.5 h. The reaction mixture was concentrated in vacuo to give residue, which was purified (PM197) to afford compound 2.454 (110 mg, 213.42 μmol, 77.42% yield, 2TFA salt) as a brown oil.

LCMS (AM11): rt=0.250 min, (288.0 [M+H]⁺), 78.60% purity.

### Synthesis of Intermediate 2.457

### 4-(4-nitro-1-(tetrahydro-2H-pyran-2-yl)-1H-indazol-6-yl)-4H-1,2,4-triazole-3-carboxamide 2.455

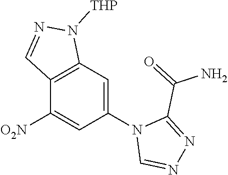

To a solution of compound 1.272 (2 g, 6.36 mmol) in THF (40 mL) was added TEA (1.29 g, 12.73 mmol) followed by 2,2,2-trichloroacetyl chloride (1.16 g, 6.36 mmol) at 0° C. The mixture was stirred at 0° C. for 2 h, the mixture was warmed to 20° C. and NH₃/MeOH (7 M, 9.09 mL) was added. The resulting mixture was stirred at 20° C. for 0.5 h. The mixture was concentrated in vacuo to give a residue. The residue was triturated with MeOH (10 mL×2) at 20° C. and filtered. The filter cake was collected and dried under vacuum to afford compound 2.455 (840 mg, 2.20 mmol, 34.54% yield) as a brown solid.

LCMS (AM14): rt=0.456 min, (358.2, [M+H]⁺), 93.59% purity

### 4-(4-nitro-1-(tetrahydro-2H-pyran-2-yl)-1H-indazol-6-yl)-4H-1,2,4-triazole-3-carbonitrile 2.456

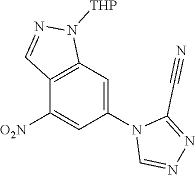

To a solution of compound 2.455 (840 mg, 2.35 mmol) in DCM (20 mL) was added TEA (713.64 mg, 7.05 mmol) followed by TFAA (987.48 mg, 4.70 mmol) at 0° C. The mixture was warmed to 20° C. and stirred for 1 h. The mixture was quenched by H₂O (10 mL) and concentrated in vacuo to give a residue, which was purified (PM108) to afford compound 2.456 (640 mg, 1.88 mmol, 79.83% yield) as a yellow solid.

LCMS (AM14): rt=0.559 min, (340.1, [M+H]⁺), 99.54% purity

### 4-(4-amino-1-(tetrahydro-2H-pyran-2-yl)-1H-indazol-6-yl)-4H-1,2,4-triazole-3-carbonitrile 2.457

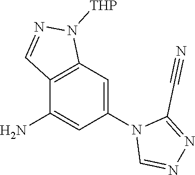

To a solution of compound 2.456 (640 mg, 1.89 mmol) in H₂O (10 mL) and EtOH (10 mL) was added Fe (737.34 mg, 13.20 mmol) and NH₄Cl (807.16 mg, 15.09 mmol). The mixture was heated to 70° C. and stirred for 1 h. The mixture was filtered and the filtrate was concentrated in vacuo to give a residue, which was purified (PM109) to afford compound 2.457 (390 mg, 1.26 mmol, 66.84% yield) as a yellow gum,

LCMS (AM12): rt=0.400 min, (310.0, [M+H]⁺), 73.45% purity

### Synthesis of Intermediate 2.463

### tert-butyl ((2S)-5-(2-((6-bromo-1-(tetrahydro-2H-pyran-2-yl)-1H-indazol-4-yl)amino)ethoxy)pentan-2-yl)carbamate 2.460

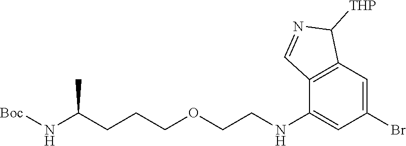

To a mixture of compound 2.430 (2.39 g, 9.72 mmol) in MeOH (30 mL) was added compound 1.381 (2.4 g, 8.10 mmol), MgSO₄ (2.93 g, 24.31 mmol) followed by HOAc (486.64 mg, 8.10 mmol). The mixture was stirred at 20° C. for 12 h, then NaBH₃CN (1.02 g, 16.21 mmol) was added. The mixture was stirred at 20° C. for 1 h. The mixture was filtered and the filtrate was concentrated in vacuo to give a residue, which was purified (PM201) to afford compound 2.460 (2.2 g, 3.93 mmol, 48.45% yield) as a yellow oil.

LCMS (AM11): rt=0.540 min, (527.2, [M+H]⁺), 93.10% purity.

### tert-butyl ((2S)-5-(2-((1-(tetrahydro-2H-pyran-2-yl)-6-(4,4,5,5-tetramethyl-1,3,2-dioxaborolan-2-yl)-1H-indazol-4-yl)amino)ethoxy)pentan-2-yl)carbamate 2.461

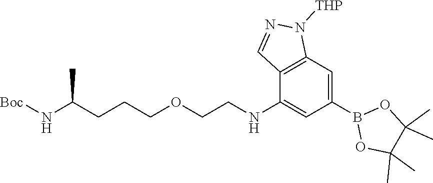

To a solution of compound 2.460 (1.7 g, 3.24 mmol) and B₂Pin₂ (985.83 mg, 3.88 mmol) in dioxane (15 mL) was added KOAc (635.01 mg, 6.47 mmol) followed by Pd(dppf)Cl₂ (236.72 mg, 323.52 μmol). The mixture was degassed and purged with N₂ (×3), then the mixture was heated to 90° C. and stirred for 2 h. The mixture was filtered and the filtrate was concentrated in vacuo to give a residue, which was purified (PM27) to afford compound 2.461 (1.8 g, 2.82 mmol, 87.17% yield) as a yellow gum.

LCMS (AM11): rt=0.610 min, (573.3, [M+H]⁺), 89.71% purity.

### tert-butyl ((2S)-5-(2-((6-(6-oxo-1,6-dihydropyridazin-4-yl)-1-(tetrahydro-2H-pyran-2-yl)-1H-indazol-4-yl)amino)ethoxy)pentan-2-yl)carbamate 2.462

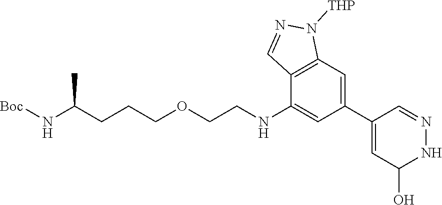

To a solution of compound 2.461 (1.8 g, 3.14 mmol) and 5-chloropyridazin-3(2H)-one (492.45 mg, 3.77 mmol) in dioxane (20 mL) and H₂O (2 mL) was added K₂CO₃ (869.03 mg, 6.29 mmol) followed by Pd(dppf)Cl₂ (230.04 mg, 314.39 μmol). The mixture was degassed and purged with N₂ (×3), then the mixture was heated to 100° C. and stirred for 12 h. The mixture was filtered and the filtrate was concentrated in vacuo to give a residue, which was purified (PM38) to afford compound 2.462 (1 g, 1.84 mmol, 58.60% yield) as a yellow solid.

LCMS (AM11): rt=0.476 min, (541.4, [M+H]⁺), 99.65% purity.

¹H NMR (400 MHz, MeOH-d₄) δ 8.37 (d, J=2.0 Hz, 1H), 8.20 (s, 1H), 7.22 (s, 1H), 7.17 (d, J=2.0 Hz, 1H), 6.49 (s, 1H), 5.82 (dd, J=10.0, 2.4 Hz, 1H), 4.01 (d, J=11.6 Hz, 1H), 3.84 (td, J=11.2, 3.2 Hz, 1H), 3.75-3.70 (m, 2H), 3.57-3.49 (m, 5H), 2.56-2.43 (m, 1H), 2.17-2.09 (m, 1H), 2.01-1.97 (m, 1H), 1.90-1.78 (m, 1H), 1.77-1.65 (m, 2H), 1.63-1.56 (m, 2H), 1.52-1.42 (m, 2H), 1.41 (s, 9H), 1.03 (d, J=6.4 Hz, 3H) ppm. (S)-5-(4-((2-((4-aminopentyl)oxy)ethyl)amino)-1H-indazol-6-yl)pyridazin-3(2H)-one 2.463

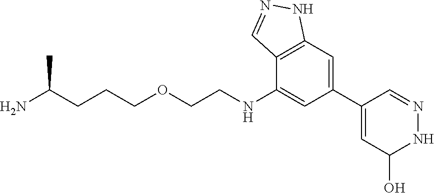 [SMILES: C[C@H](N)CCCOCCNc1cc(C2=CC(O)NN=C2)cc2[nH]ncc12]

To a solution of compound 2.462 (950 mg, 1.76 mmol) in MeOH (10 mL) was added HCl/dioxane (4 M, 15 mL). The mixture was stirred at 20° C. for 2 h. The mixture was concentrated in vacuo to afford compound 2.463 (0.9 g, crude, HCl salt) as a red gum.

LCMS (AM11): rt=0.251 min, (357.1 [M+H]⁺), 93.9% purity.

### Synthesis of Intermediate 2.468

### tert-butyl ((2S)-5-(2-((6-(isoxazol-4-yl)-1-(tetrahydro-2H-pyran-2-yl)-1H-indazol-4-yl)amino)ethoxy)pentan-2-yl)carbamate 2.467

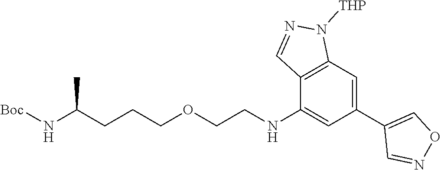

To a mixture of compound 2.460 (390 mg, 742.18 μmol) and 4-(4,4,5,5-tetramethyl-1,3,2-dioxaborolan-2-yl)isoxazole (173.69 mg, 890.62 μmol) in DMF (20 mL) and H₂O (2 mL) was added KF (86.24 mg, 1.48 mmol) followed by Pd(dppf)Cl₂ (54.31 mg, 74.22 μmol). The mixture was degassed and purged with N₂ (×3), then the mixture was heated to 70° C. and stirred for 10 min. The reaction mixture was poured into water (60 mL) and extracted with EA (40 mL×3). The combined organic phase was washed (brine, 20 mL×3), dried (Na₂SO₄), filtered and concentrated in vacuo to give a residue, which was purified (PM24) to afford compound 2.467 (280 mg, 530.97 μmol, 71.54% yield) as a yellow gum.

LCMS (AM11): rt=0.508 min, (514.4, [M+H]⁺), 97.47% purity.

¹H NMR (400 MHz, CHCl₃-d) δ 9.12 (s, 1H), 8.91 (s, 1H), 8.14 (s, 1H), 7.13 (s, 1H), 6.48 (s, 1H), 6.37 (d, J=7.6 Hz, 1H), 5.76 (dd, J=10.0, 2.0 Hz, 1H), 4.03 (d, J=11.6 Hz, 1H), 3.84 (td, J=11.2, 2.4 Hz, 1H), 3.75-3.70 (m, 2H), 3.54-3.50 (m, 4H), 2.56-2.4 (m, 1H), 2.18-2.09 (m, 1H), 2.01-1.94 (m, 1H), 1.91-1.71 (m, 2H), 1.70-1.55 (m, 4H), 1.52-1.44 (m, 2H), 1.41 (s, 9H), 1.04 (d, J=6.8 Hz, 3H) ppm.

### (S)-N-(2-((4-aminopentyl)oxy)ethyl)-6-(isoxazol-4-yl)-1H-indazol-4-amine 2.468

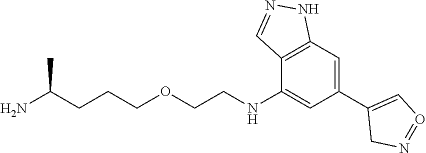

To a solution of compound 2.467 (260 mg, 506.20 μmol) in DCM (20 mL) was added TFA (7.8 mL, 105.35 mmol) at 20° C. The mixture was stirred at 20° C. for 10 min. The mixture was concentrated in vacuo to afford compound 2.468 (400 mg, crude, TFA salt) as a yellow gum.

LCMS (AM11): rt=0.262 min, (330.2, [M+H]⁺), 74.40% purity.

### Synthesis of Intermediate 2.479

### 6-(3-chloropyridin-4-yl)-1-(tetrahydro-2H-pyran-2-yl)-1H-indazol-4-amine 2.479

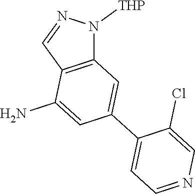

To a solution of compound 1.952 (500 mg, 1.46 mmol) and 4-bromo-3-chloro-pyridine (CAS 73583-41-2, 308.37 mg, 1.60 mmol) in dioxane (8 mL) and H₂O (0.8 mL) was added K₂CO₃ (503.33 mg, 3.64 mmol) followed by Pd(dppf)Cl₂ (106.59 mg, 145.68 μmol). The mixture was degassed and purged with N₂ (×3), the mixture was heated to 100° C. and stirred for 12 h. The reaction mixture was cooled to 20° C., diluted with DCM (40 mL) and filtered. The filtrate was concentrated in vacuo to give a residue, which was purified (PM42) to afford compound 2.479 (0.325 g, 868.75 μmol, 59.64% yield) as a brown oil.

LCMS (AM12): rt=0.443 min, (329.2, [M+H]⁺), 87.89% purity.

### Synthesis of Intermediate 2.488

### methyl 6-bromo-1-(tetrahydro-2H-pyran-2-yl)-1H-indazole-4-carboxylate 2.483

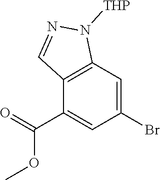

To a solution of methyl 6-bromo-1H-indazole-4-carboxylate (CAS 885518-49-0, 10 g, 39.21 mmol) and 3,4-dihydro-2H-pyran (9.89 g, 117.62 mmol) in THF (100 mL) was added TsOH (675.12 mg, 3.92 mmol). The mixture was heated to 50° C. and stirred for 12 h. The reaction mixture was concentrated in vacuo to give a residue. The residue was triturated with EA (30 mL×2) at 20° C. and filtered. The filter cake was collected and dried under vacuum to afford compound 2.483 (14.3 g, crude) as a white solid.

LCMS (AM12): rt=0.608 min, (360.9, [M+Na]⁺), 98.46% purity.

¹H NMR (CHCl₃-d, 400 MHz) 5 8.49 (s, 1H), 8.06-8.00 (m, 2H), 5.72 (dd, J=9.2, 2.8 Hz, 1H), 4.04-3.99 (m, 4H), 3.82-3.73 (m, 1H), 2.58-2.48 (m, 1H), 2.23-20.5 (m, 2H), 1.83-1.68 (m, 3H) ppm.

### methyl 6-amino-1-(tetrahydro-2H-pyran-2-yl)-1H-indazole-4-carboxylate 2.484

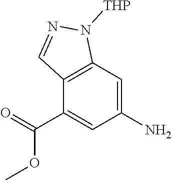

To a mixture of compound 2.483 (14.3 g, 42.16 mmol) in DMSO (100 mL) was added K₂CO₃ (17.48 g, 126.48 mmol), NH₃·H₂O (11.98 g, 126.48 mmol) and L-proline (2.91 g, 25.30 mmol) followed by CuI (2.41 g, 12.65 mmol). The mixture was degassed and purged with N₂ (×3), then heated to 75° C. and stirred for 12 h. The reaction mixture was poured into water (300 mL) and extracted with EA (200 mL×3). The combined organic phase was washed (brine, 100 mL×5), dried (Na₂SO₄), filtered and concentrated in vacuo to afford compound 2.484 (6 g, 19.29 mmol, 45.75% yield) as a white solid.

LCMS (AM12): rt=0.393 min, (276.2, [M+H]⁺), 88.56% purity.

### methyl 1-(tetrahydro-2H-pyran-2-yl)-6-(4H-1,2,4-triazol-4-yl)-1H-indazole-4-carboxylate 2.485

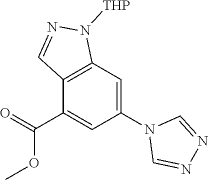

To a solution of compound 2.484 (6 g, 21.79 mmol) and N′-formylformohydrazide (5.76 g, 65.38 mmol) in pyridine (100 mL) was added TEA (15.44 g, 152.56 mmol) followed by TMSCl (35.52 g, 326.91 mmol) dropwise at 20° C. The mixture was heated to 100° C. and stirred for 12 h. The mixture was concentrated in vacuo to give a residue. The residue was triturated with MeOH (50 mL×3) and filtered. The filter cake was collected and dried under vacuum to afford compound 2.485 (6 g, 17.71 mmol, 81.24% yield) as an off-white solid.

LCMS (AM16): rt=0.532 min, (314.2, [M+H]⁺), 96.62% purity.

¹H NMR (400 MHz, CHCl₃-d) δ 8.67-8.53 (m, 3H), 7.94 (d, J=1.6 Hz, 1H), 7.89 (s, 1H), 5.87-5.77 (m, 1H), 4.07 (s, 3H), 4.04-3.95 (m, 1H), 3.84-3.74 (m, 1H), 2.59-2.46 (m, 1H), 2.23-2.12 (m, 2H), 1.89-1.70 (m, 3H) ppm.

### 1-(tetrahydro-2H-pyran-2-yl)-6-(4H-1,2,4-triazol-4-yl)-1H-indazole-4-carboxamide 2.486

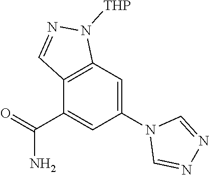

A solution of compound 2.485 (3 g, 9.16 mmol) in NH₃/MeOH (7 M, 20 mL) was heated to 80° C. and stirred for 48 h. The mixture was concentrated in vacuo to afford compound 2.486 (2.5 g, 8.00 mmol, 81.62% yield) as a white solid.

LCMS (AM11): rt=0.398 min, (313.2, [M+H]⁺), 96.45% purity.

### 1-(tetrahydro-2H-pyran-2-yl)-6-(4H-1,2,4-triazol-4-yl)-1H-indazole-4-carbonitrile 2.487

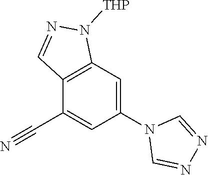

To a solution of compound 2.486 (1.25 g, 4.00 mmol) in pyridine (30 mL) was added TFAA (2.52 g, 12.01 mmol). The mixture was stirred at 20° C. for 4 h. The mixture was quenched by water (10 mL) and concentrated in vacuo to give a residue, which was triturated with ACN (20 mL×2) at 20° C. and filtered. The filter cake was collected and dried under vacuum to afford compound 2.487 (950 mg, 3.19 mmol, 79.68% yield) as an off-white solid.

LCMS (AM16): rt=0.518 min, (295.2, [M+H]⁺), 98.85% purity.

### (1-(tetrahydro-2H-pyran-2-yl)-6-(4H-1,2,4-triazol-4-yl)-1H-indazol-4-yl)methanamine 2.488

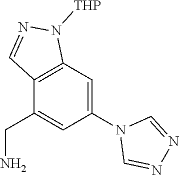

To a mixture of compound 2.487 (1 g, 3.40 mmol) in MeOH (40 mL) was added NH₃·H₂O (965.49 mg, 10.19 mmol) and Raney-Ni (300 mg, 3.50 mmol). The mixture was degassed and purged with H₂ (×3), then stirred at 20° C. for 1 h under H₂ (15 psi). The catalyst was filtered and the filtrate was concentrated in vacuo to afford compound 2.488 (940 mg, 3.15 mmol, 92.73% yield) as a yellow solid.

LCMS (AM11): rt=0.250 min, (298.9, [M+H]⁺), 89.09% purity.

¹H NMR (400 MHz, DMSO-d₆) δ 9.17 (s, 2H), 8.35 (s, 1H), 7.96 (s, 1H), 7.50 (d, J=0.8 Hz, 1H), 5.86 (dd, J=9.6, 2.0 Hz, 1H), 4.09 (s, 2H), 3.91 (br d, J=11.6 Hz, 1H), 3.81-3.70 (m, 1H), 2.47-2.38 (m, 1H), 2.02-1.91 (m, 2H), 1.79-1.61 (m, 1H), 1.65-1.55 (m, 2H) ppm.

### Synthesis of Intermediate 2.490

### tert-butyl (3,5-difluoro-4-(trifluoromethoxy)benzyl)(4-(2-(((1-(tetrahydro-2H-pyran-2-yl)-6-(4H-1,2,4-triazol-4-yl)-1H-indazol-4-yl)methyl)amino)ethoxy)butyl)carbamate 2.490

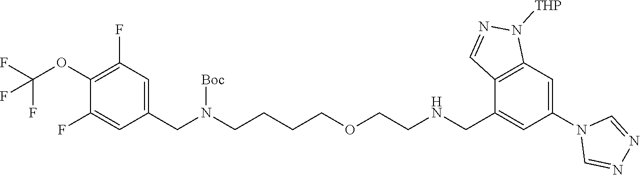

To a mixture of compound 2.488 (100 mg, 335.19 μmol) in MeOH (1.5 mL) was added compound 1.941 (147.95 mg, 335.19 μmol) and MgSO₄ (201.73 mg, 1.68 mmol). The mixture was stirred at 20° C. for 2 h, then NaBH₃CN (57.45 mg, 914.14 μmol) was added and the mixture was stirred at 20° C. for 1 h. The reaction mixture was filtered and the filtrate was concentrated in vacuo to give a residue, which was purified (PM199) to afford a solution. The solution was basified to pH=9 by saturated NaHCO₃ aqueous solution and extracted with EA (30 mL×3). The combined organic phase was washed by brine (20 mL×2), dried (Na₂SO₄), filtered and concentrated in vacuo to afford compound 2.490 (90 mg, 118.22 μmol, 35.27% yield) as a yellow oil.

LCMS (AM14): rt=0.576 min, (724.5, [M+H]⁺), 95.07% purity.

### Synthesis of Intermediate 2.498

### methyl 3-iodo-4-nitro-1H-indazole-6-carboxylate 2.491

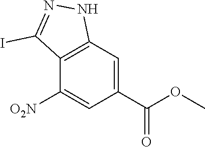 [SMILES: COC(=O)c1cc([N+](=O)[O-])c2c(I)n[nH]c2c1]

To a solution of methyl 4-nitro-1H-indazole-6-carboxylate (CAS 72922-61-3, 2 g, 9.04 mmol) in DMF (25 mL) was added NIS (2.24 g, 9.95 mmol) at 0° C. and stirred for 0.5 h, then the mixture was heated to 60° C. and stirred for 3 h. The mixture was poured into water (150 mL) and filtered. The filter cake was collected and dried under vacuum to afford compound 2.491 (3 g, 8.48 mmol, 93.82% yield) as a yellow solid.

LCMS (AM11): rt=0.405 min, (347.6, [M+H]⁺), 98.15% purity.

¹H NMR (400 MHz, DMSO-d₆) δ 14.65 (s, 1H), 8.49 (s, 1H), 8.24 (s, 1H), 3.95 (s, 3H) ppm.

### methyl 3-iodo-4-nitro-1-(tetrahydro-2H-pyran-2-yl)-1H-indazole-6-carboxylate 2.492

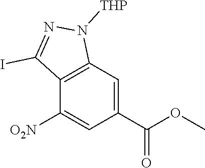

To a solution of compound 2.491 (3 g, 8.64 mmol) in THF (35 mL) was added 3,4-dihydro-2H-pyran (2.18 g, 25.93 mmol) and TsOH (148.85 mg, 864.39 μmol). The mixture was heated to 60° C. and stirred for 3 h. The mixture was diluted with EA (50 ml) and washed with saturated NaHCO₃ aqueous solution (20 mL×2) and brine (20 mL×3), then the organic layers was dried (Na₂SO₄), filtered and concentrated in vacuo to give a residue, which was triturated with MTBE (5 mL) at 20° C. and filtered. The filter cake was collected and dried under vacuum to afford compound 2.492 (2.9 g, 6.73 mmol, 77.81% yield) as a light-yellow solid.

¹H NMR (400 MHz, DMSO-d₆) δ 8.78 (s, 1H), 8.30 (s, 1H), 6.23 (dd, J=9.2 Hz, 2.0 Hz, 1H), 3.97 (s, 3H), 3.88-3.82 (m, 2H), 2.38-2.32 (m, 1H), 2.04 (d, J=10.0 Hz, 2H), 1.83-1.72 (m, 1H), 1.61 (d, J=2.4 Hz, 2H) ppm.

### methyl 3-((tert-butoxycarbonyl)amino)-4-nitro-1-(tetrahydro-2H-pyran-2-yl)-1H-indazole-6-carboxylate 2.493

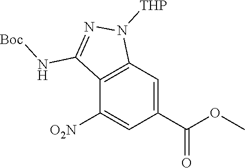

To a solution of compound 2.492 (1.9 g, 4.41 mmol) in dioxane (20 mL) was added tert-butyl carbamate (1.14 g, 9.69 mmol), Xantphos (509.94 mg, 881.30 μmol), Cs₂CO₃ (2.87 g, 8.81 mmol) and Pd₂(dba)₃ (403.51 mg, 440.65 μmol). The mixture was degassed and purged with N₂ (×3). The mixture was heated for 100° C. and stirred for 12 h. The mixture was filtered and the filtrate was concentrated in vacuo to give a residue, which was purified (PM27) to afford compound 2.493 (300 mg, 711.94 μmol, 16.16% yield) as a light-yellow solid.

LCMS (AM11): rt=0.510 min, (443.0, [M+Na]⁺), 99.77% purity.

¹H NMR (400 MHz, DMSO-d₆) δ 9.85 (s, 1H), 8.73 (s, 1H), 8.25 (s, 1H), 6.16 (dd, J=7.6 Hz, 2.0 Hz, 1H), 3.97 (s, 3H), 3.88-3.82 (m, 2H), 2.35-2.30 (m, 1H), 2.05-1.98 (m, 2H), 1.81-1.73 (m, 1H), 1.63-1.55 (m, 2H), 1.39 (s, 9H) ppm.

### methyl 3-((tert-butoxycarbonyl)(methyl)amino)-4-nitro-1-(tetrahydro-2H-pyran-2-yl)-1H-indazole-6-carboxylate 2.494

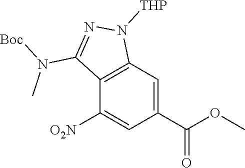

To a solution of compound 2.493 (720 mg, 1.71 mmol) in DMF (10 mL) was added Cs₂CO₃ (1.39 g, 4.28 mmol) followed by a solution of CH₃I (364.62 mg, 2.57 mmol) in DMF (0.5 mL) at 0° C. The mixture was stirred at 20° C. for 0.5 h. The mixture was poured into water (30 mL) and filtered. The filtrate was concentrated in vacuo to afford compound 2.494 (720 mg, 1.66 mmol, 96.77% yield) as a yellow solid.

LCMS (AM11): rt=0.541 min, (457.1, [M+Na]⁺), 97.46% purity.

¹H NMR (400 MHz, DMSO-d₆) δ 8.76 (t, J=8.0 Hz, 1H), 8.43 (s, 0.5H), 8.29 (s, 0.5H), 6.21 (d, J=8.8 Hz, 1H), 3.98 (s, 3H), 3.90-3.81 (m, 2H), 3.40-3.32 (m, 3H), 2.37-2.31 (m, 1H), 2.08-2.00 (m, 2H), 1.83-1.73 (m, 1H), 1.64-1.57 (m, 2H), 1.44 (s, 4H), 1.15 (s, 5H) ppm.

### 3-((tert-butoxycarbonyl)(methyl)amino)-4-nitro-1-(tetrahydro-2H-pyran-2-yl)-1H-indazole-6-carboxylic acid 2.495

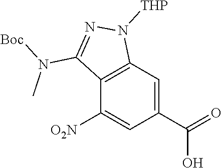

To a solution of compound 2.494 (700 mg, 1.61 mmol) in MeOH (15 mL) and H₂O (4.5 mL) was added NaOH (257.78 mg, 6.45 mmol). The mixture was stirred at 20° C. for 1 h. The mixture was concentrated in vacuo, acidified to pH=6 by 1M HCl aqueous solution and filtered. the filter cake was collected and dried under vacuum to afford compound 2.495 (660 mg, 1.57 mmol, 97.43% yield) as a light-yellow solid.

LCMS (AM11): rt=0.491 min, (443.1, [M+Na]⁺), 93.89% purity.

### tert-butyl (6-amino-4-nitro-1-(tetrahydro-2H-pyran-2-yl)-1H-indazol-3-yl)(methyl)carbamate 2.496

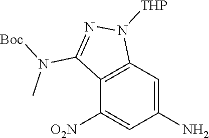

To a solution of compound 2.495 (650 mg, 1.55 mmol) in dioxane (15 mL) was added TEA (645.59 μL, 4.64 mmol) and DPPA (510.58 mg, 1.86 mmol). The mixture was stirred at 20° C. for 3 h, then H₂O (4.5 mL) was added. The mixture was heated to 100° C. and stirred for 3 h. The mixture was poured into water (30 mL) and extracted with EA (20 mL×3). The combined organic phase was washed (brine, 20 mL×2), dried (Na₂SO₄), filtered and concentrated in vacuo to give a residue, which was purified (PM24) to afford compound 2.496 (430 mg, 1.06 mmol, 68.40% yield) as a yellow solid.

LCMS (AM11): rt=0.473 min, (414.0, [M+Na]⁺), 96.26% purity.

### tert-butyl methyl(4-nitro-1-(tetrahydro-2H-pyran-2-yl)-6-(4H-1,2,4-triazol-4-yl)-1H-indazol-3-yl)carbamate 2.497

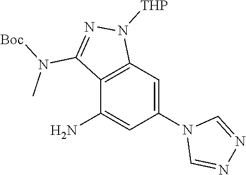

To a solution of compound 2.496 (430 mg, 1.10 mmol) in pyridine (12 mL) was added N,N′-diformylhydrazine (484.71 mg, 5.50 mmol), TEA (778.14 mg, 7.69 mmol) and TMSCl (1.79 g, 16.48 mmol) dropwise at 0° C. The mixture was heated to 100° C. and stirred for 12 h. The reaction mixture was cooled to 20° C. and quenched by water (2 mL) at 0° C. The mixture was concentrated in vacuo to give a residue, which was purified (PM204) to compound 2.497 (370 mg, 834.36 μmol, 75.95% yield) as a yellow solid.

LCMS (AM11): rt=0.436 min, (444.4, [M+H]⁺), 100.00% purity.

### tert-butyl (4-amino-1-(tetrahydro-2H-pyran-2-yl)-6-(4H-1,2,4-triazol-4-yl)-1H-indazol-3-yl)(methyl)carbamate 2.498

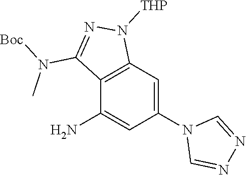

A solution of compound 2.497 (370 mg, 834.36 μmol) in MeOH (5 mL) was degassed and purged with N₂ (×3), then Pd/C (80 mg, 10% purity) was added. The mixture was degassed and purged with H₂ (×3) and stirred at 20° C. for 1 h under H₂ balloon (15 psi). The mixture was filtered and the filtrate was concentrated in vacuo to give a residue, which was purified (PM238) to afford compound 2.498 (203 mg, 490.96 μmol, 58.84% yield) as a yellow solid.

LCMS (AM11): rt=0.509 min, (414.2, [M+H]⁺), 100.00% purity.

¹H NMR (400 MHz, DMSO-d₆) δ 9.00 (s, 2H), 7.13 (s, 1H), 6.45 (s, 1H), 5.69 (d, J=8.0 Hz, 1H), 5.61 (s, 2H), 3.92-3.86 (m, 1H), 3.75-3.69 (m, 1H), 3.19 (s, 3H), 2.35-2.28 (m, 1H), 2.06-1.99 (m, 1H), 1.92-1.85 (m, 1H), 1.75-1.66 (m, 1H), 1.57 (s, 2H), 1.36 (s, 9H) ppm.

### Synthesis of Intermediate 2.500

### tert-butyl (4-((3-(3-((tert-butoxycarbonyl)(3-chloro-4-(trifluoromethoxy)benzyl)amino)propanamido)propyl)amino)-1-(tetrahydro-2H-pyran-2-yl)-6-(4H-1,2,4-triazol-4-yl)-1H-indazol-3-yl)(methyl)carbamate 2.500

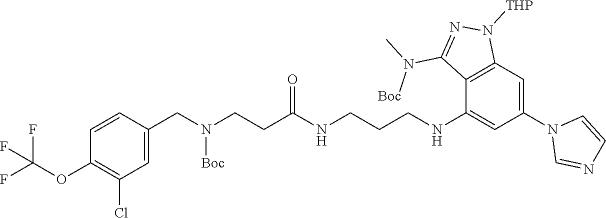

To a solution of compound 2.498 (35 mg, 84.65 μmol) in MeOH (1 mL) was added compound 1.950 (49.83 mg, 110.04 μmol) and AcOH (8.71 mg, 145.11 μmol). The mixture was stirred at 20° C. for 12 h, then NaBH₃CN (26.60 mg, 423.24 μmol) was added and the mixture was stirred at 20° C. for 2 h. The mixture was filtered and the filtrate was purified (PM179) to afford compound 2.500 (40 mg, 47.04 μmol, 55.57% yield) as a white solid.

LCMS (AM11): rt=0.554 min. (850.7[M+H]⁺), 100.00% purity.

### Synthesis of Intermediate 2.511

### 4-amino-1H-indazole-6-carboxylic acid 2.511

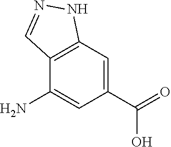 [SMILES: Nc1cc(C(=O)O)cc2[nH]ncc12]

To a solution of methyl 4-amino-1H-indazole-6-carboxylate (CAS 885518-51-4, 400 mg, 2.09 mmol) in MeOH (10 mL) and H₂O (3 mL) was added NaOH (251.05 mg, 6.28 mmol). The mixture was stirred at 20° C. for 1 h. The mixture was poured into water (40 mL) and extracted with EA (50 ml×3). The combined organic phase was washed (brine, 20 ml), dried (Na₂SO₄), filtered and concentrated in vacuo to afford compound 2.511 (70 mg, 297.25 μmol, 23.40% yield) as a brown oil.

¹H NMR (400 MHz, DMSO-d₆) δ 13.06-12.81 (m, 1H), 12.70-12.40 (m, 1H), 8.15 (s, 1H), 7.26 (s, 1H), 6.73 (s, 1H), 5.95 (s, 2H) ppm.

### Synthesis of Intermediate 2.514

(E)-tert-butyl 3-(4-amino-1-(tetrahydro-2H-pyran-2-yl)-1H-indazol-6-yl)acrylate 2.513

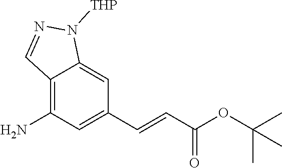

To a solution of compound 1.381 (300 mg, 1.01 mmol) and tert-butyl acrylate (194.74 mg, 1.52 mmol) in ACN (6 mL) was added TEA (410.00 mg, 4.05 mmol), tris-o-tolylphosphine (61.66 mg, 202.59 μmol) followed by Pd(OAc)₂ (22.74 mg, 101.30 μmol). The mixture was degassed and purged with N₂ (×3), then the mixture was heated to 90° C. and stirred for 6 h. The reaction mixture was poured into water (30 mL) and extracted with EA (20 mL×3). The combined organic phase was washed by brine (30 mL×2), dried (Na₂SO₄), filtered and concentrated in vacuo to give a residue, which was purified (PM27) to afford compound 2.513 (340 mg, 917.28 μmol, 90.55% yield) as a yellow gum.

LCMS (AM11): rt=0.487 min, (344.3, [M+H]⁺), 92.65% purity.

¹H NMR (400 MHz, DMSO-d₆) δ 8.14 (s, 1H), 7.51 (d, J=15.6 Hz, 1H), 7.16 (s, 1H), 6.45 (s, 1H), 6.40 (d, J=16.0 Hz, 1H), 5.93 (s, 2H), 5.74 (dd, J=10.0, 2.4 Hz, 1H), 3.93-3.85 (m, 1H), 3.75-3.69 (m, 1H), 2.44-2.33 (m, 1H), 2.08-2.00 (m, 1H), 1.95-1.87 (m, 1H), 1.78-1.65 (m, 1H), 1.60-1.54 (m, 2H), 1.50 (s, 9H) ppm.

### tert-butyl 3-(4-amino-1-(tetrahydro-2H-pyran-2-yl)-1H-indazol-6-yl)propanoate 2.514

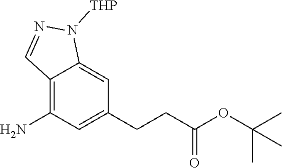

A solution of Pd/C (200 mg, 10% purity) in EA (20 mL) was degassed and purged with N₂ (×3), then compound 2.513 (340 mg, 990.04 μmol) was added in one portion. The mixture was degassed and purged with H₂ (×3) and stirred at 20° C. for 1 h under H₂ balloon (15 psi). The mixture was filtered and the filtrate was concentrated in vacuo to afford compound 2.514 (310 mg, 858.74 μmol, 86.74% yield) as a brown gum.

LCMS (AM11): rt=0.437 min, (346.4, [M+H]⁺), 95.69% purity.

### Synthesis of Intermediate 2.529

### tert-butyl (3-((2-oxoethoxy)methyl)cyclobutyl)carbamate 2.527

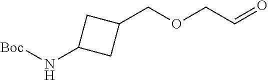 [SMILES: CC(C)(C)OC(=O)NC1CC(COCC=O)C1]

To a solution of oxalyl dichloride (517.40 mg, 4.08 mmol) in DCM (2 mL) was added a solution of DMSO (477.75 mg, 6.11 mmol) in DCM (1 mL) dropwise at −70° C. under N₂. The mixture was stirred at −70° C. for 0.5 h, then a solution of compound 1.856 (0.5 g, 2.04 mmol) in DCM (1 mL) was dropwise added at −70° C. and the resulting mixture was stirred at −70° C. for 0.5 h. Then TEA (1.24 g, 12.23 mmol) was added at −70° C. and the reaction was stirred at −70° C. for 10 min. The mixture was diluted with DCM (2 mL) and filtered. The filtrate was concentrated in vacuo to give a residue, which was purified (PM27) to afford compound 2.527 (0.5 g, crude) as a yellow oil.

¹H NMR (400 MHz, CHCl₃-d) δ 9.74-9.70 (m, 1H), 4.09-4.03 (m, 2H), 3.50-3.40 (m, 2H), 3.15-3.06 (m, 1H), 2.50-2.39 (m, 3H), 1.70-1.59 (m, 2H), 1.43 (s, 9H) ppm.

### tert-butyl (3-((2-((6-(1,2,3-thiadiazol-5-yl)-1H-indazol-4-yl)amino)ethoxy)methyl)cyclobutyl)carbamate 2.528

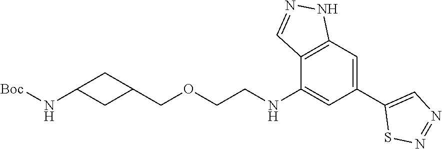 [SMILES: CC(C)(C)OC(=O)NC1CC(COCCNc2cc(-c3cnns3)cc3[nH]ncc23)C1]

To a mixture of compound 1.995 (200 mg, 920.60 μmol) and compound 2.527 (447.96 mg, 1.84 mmol) in MeOH (5 mL) was added MgSO₄ (554.06 mg, 4.60 mmol) and HOAc (82.92 mg, 1.38 mmol). The mixture was stirred at 20° C. for 12 h, then NaBH₃CN (115.70 mg, 1.84 mmol) was added. The resulting mixture was stirred at 20° C. for 1 h. The reaction mixture was poured into water (40 mL) and extracted with EA (40 mL×3). The combined organic phase was washed (brine, 20 mL×2), dried (Na₂SO₄), filtered and concentrated in vacuo to give a residue, which was purified (PM24) to afford compound 2.528 (150 mg, 283.43 μmol, 30.79% yield) as a yellow gum.

LCMS (AM11): rt=0.459 min, (445.2, [M+H]⁺), 84.22% purity.

### N-(2-((3-aminocyclobutyl)methoxy)ethyl)-6-(1,2,3-thiadiazol-5-yl)-1H-indazol-4-amine 2.529

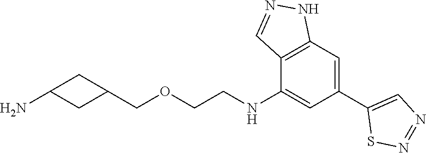 [SMILES: NC1CC(COCCNc2cc(-c3cnns3)cc3[nH]ncc23)C1]

To a solution of compound 2.528 (145 mg, 326.17 μmol) in DCM (20 mL) was added TFA (6.16 g, 54.02 mmol) at 20° C. The mixture was stirred at 20° C. for 0.5 h. The mixture was concentrated in vacuo to afford compound 2.529 (150 mg, crude, TFA salt) as a yellow gum.

LCMS (AM11): rt=0.292 min, (334.9, [M+H]⁺), 76.10% purity.

### Synthesis of Intermediate 2.531

### tert-butyl (S)-(5-(2-((6-(1,2,3-thiadiazol-5-yl)-1H-indazol-4-yl)amino)ethoxy)pentan-2-yl)carbamate 2.530

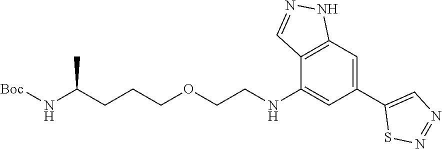 [SMILES: C[C@@H](CCCOCCNc1cc(-c2cnns2)cc2[nH]ncc12)NC(=O)OC(C)(C)C]

To a mixture of compound 2.430 (293.59 mg, 1.20 mmol) and compound 1.995 (200 mg, 920.60 μmol) in MeOH (20 mL) was added MgSO₄ (554.05 mg, 4.60 mmol) and HOAc (66.34 mg, 1.10 mmol). The mixture was stirred at 20° C. for 12 h, then NaBH₃CN (173.56 mg, 2.76) was added in portions at 0° C. The mixture was stirred at 20° C. for 1 h. The reaction mixture was diluted with water (40 mL) and extracted with EA (30 mL×3). The combined organic phase was washed by brine (30 mL×3), dried (Na₂SO₄), filtered and concentrated in vacuo to give a residue, which was purified (PM182) to afford compound 2.530 (220 mg, 469.00 μmol, 50.95% yield) as a yellow gum.

LCMS (AM22): rt=0.528 min, (447.3, [M+H]⁺), 95.21% purity.

### (S)-N-(2-((4-aminopentyl)oxy)ethyl)-6-(1,2,3-thiadiazol-5-yl)-1H-indazol-4-amine 2.531

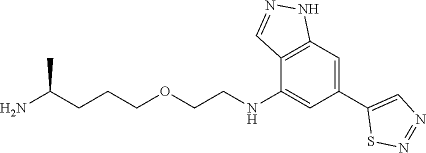 [SMILES: C[C@H](N)CCCOCCNc1cc(-c2cnns2)cc2[nH]ncc12]

To a solution of compound 2.530 (220 mg, 492.65 μmol) in MeOH (2 mL) was added HCl/dioxane (4 M, 3 mL). The mixture was stirred at 20° C. for 0.5 h. The mixture was concentrated in vacuo to afford compound 2.531 (200 mg, 465.84 μmol, 94.56% yield, HCl salt) as a yellow solid.

LCMS (AM22): rt=0.378 min, (347.2, [M+H]⁺), 97.68% purity.

### Synthesis of Intermediate 2.537

### 2-methyl-2-(4-nitro-1-(tetrahydro-2H-pyran-2-yl)-1H-indazol-6-yl)propanenitrile 2.534

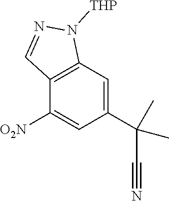

To a solution of compound 2.451 (300 mg, 1.05 mmol) in DMF (4 mL) was added CH₃I (743.69 mg, 5.24 mmol), then Cs₂CO₃ (1.02 g, 3.14 mmol) was added in portions at 0° C. The mixture was stirred at 20° C. for 2 h. The mixture was poured into saturated NH₄Cl aqueous solution (45 mL) and extracted with EA (30 mL×3). The combined organic phase was washed by brine (60 mL×2), dried (Na₂SO₄), filtered and concentrated in vacuo to give a residue, which was purified (PM26) to afford compound 2.534 (290 mg, 922.57 μmol, 88.04% yield) as a yellow solid.

LCMS (AM11): rt=0.492 min, (337.1, [M+Na]*), 100% purity.

¹H NMR (400 MHz, DMSO-d₆) δ 8.58 (s, 1H), 8.41 (s, 1H), 8.32 (s, 1H), 6.14 (d, J=8.8 Hz, 1H), 3.93-3.86 (m, 1H), 3.78-3.85 (m, 1H), 2.46-2.37 (m, 1H), 2.13-2.06 (m, 1H), 2.02-1.97 (m, 1H), 1.85 (s, 6H), 1.80-1.70 (m, 1H), 1.65-1.54 (m, 2H) ppm.

### 2-(4-amino-1-(tetrahydro-2H-pyran-2-yl)-1H-indazol-6-yl)-2-methylpropanenitrile 2.535

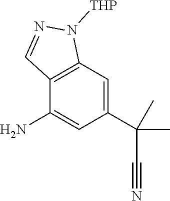

To a solution of compound 2.534 (240 mg, 763.51 μmol) in MeOH (3 mL) and H₂O (1 mL) was added NH₄Cl (326.72 mg, 6.11 mmol) followed by Fe (255.83 mg, 4.58 mmol). The mixture was stirred at 50° C. for 1.5 h. The reaction mixture was cooled to 20° C., diluted with EA (30 mL), filtered and washed with EA (10 mL×2). The filtrate was poured into water (40 mL) and extracted with EA (30 mL×3). The combined organic phase was washed by brine (40 mL×2), dried (Na₂SO₄), filtered and concentrated in vacuo to afford compound 2.535 (245 mg, crude) as a brown gum.

LCMS (AM14): rt=0.629 min, (285.2, [M+H]⁺), 90.41% purity.

### tert-butyl (4-(2-((6-(2-cyanopropan-2-yl)-1-(tetrahydro-2H-pyran-2-yl)-1H-indazol-4-yl)amino)ethoxy)butyl)carbamate 2.536

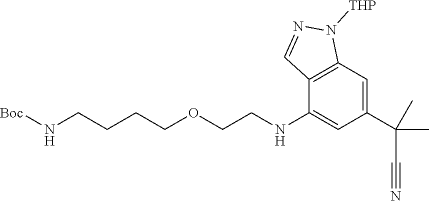

To a solution of compound 2.535 (240 mg, 844.01 μmol) in MeOH (5 mL) was added tert-Butyl (4-(2-aminoethoxy)butyl)carbamate (CAS 2830620-37-4, 234.25 mg, 1.01 mmol), MgSO₄ (304.78 mg, 2.53 mmol) and AcOH (101.37 mg, 1.69 mmol). The mixture was stirred at 20° C. for 12 h, then NaBH₃CN (106.08 mg, 1.69 mmol) was added. The resulting mixture was stirred at 20° C. for 1 h. The reaction mixture was poured into water (30 mL) and extracted with EA (30 mL×3). The combined organic phase was washed (brine, 40 mL×2), dried (Na₂SO₄), filtered and concentrated in vacuo to give a residue, which was purified (PM24) to afford compound 2.536 (298 mg, 558.37 μmol, 66.16% yield) as a yellow gum.

LCMS (AM11): rt=0.523 min, (500.4, [M+H]⁺), 93.62% purity.

### 2-(4-((2-(4-aminobutoxy)ethyl)amino)-1H-indazol-6-yl)-2-methylpropanenitrile 2.537

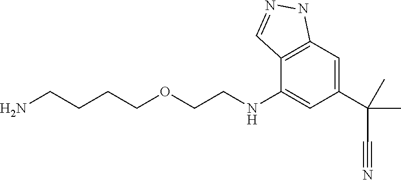 [SMILES: CC(C)(C#N)c1cc(NCCOCCCCN)c2cn[nH]c2c1]

To a solution of compound 2.536 (120 mg, 240.17 μmol) in DCM (6 mL) was added TFA (1.2 mL, 16.21 mmol). The mixture was stirred at 20° C. for 2 h. The mixture was concentrated in vacuo to afford compound 2.537 (260 mg, crude) as a brown gum.

LCMS (AM22): rt=0.289 min, (316.2, [M+H]⁺), 77.55% purity.

### Synthesis of Intermediate 2.548

### 5-(4-nitro-1-(tetrahydro-2H-pyran-2-yl)-1H-indazol-6-yl)-1,2,3-thiadiazole 2.544

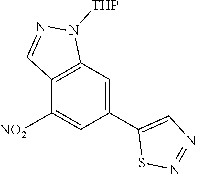

To a solution of compound 1.994 (500 mg, 2.02 mmol) in THF (8 mL) was added DHP (510.34 mg, 6.07 mmol) followed by TsOH·H₂O (38.47 mg, 202.24 μmol). The mixture was stirred at 60° C. for 0.5 h. The reaction mixture was added into saturated NaHCO₃ aqueous solution (15 mL) at 20° C., and then extracted with EA (10 mL×3). The combined organic layers were washed (brine, 15 mL×3), dried (Na₂SO₄), filtered and concentrated in vacuo to give a residue. The residue was triturated with MeOH (3 mL), filtered and washed with MeOH (1 mL×2). The filter cake was collected and dried under vacuum to afford compound 2.544 (450 mg, 1.32 mmol, 65.14% yield) as a yellow solid.

LCMS (AM12): rt=0.570 min, (332.0, [M+H]⁺), 97.64% purity.

### 1-(tetrahydro-2H-pyran-2-yl)-6-(1,2,3-thiadiazol-5-yl)-1H-indazol-4-amine 2.545

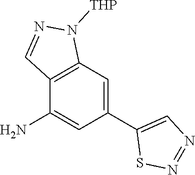

To a solution of compound 2.544 (428 mg, 1.29 mmol) in MeOH (9 mL) was added NH₄Cl (552.75 mg, 10.33 mmol) in H₂O (3 mL) followed by Fe (432.81 mg, 7.75 mmol) at 60° C. The mixture was stirred at 60° C. for 1.5 h. The mixture was filtered then the reaction mixture was added into water (30 mL) and extracted with EA (30 mL×3). The combined organic phases were washed (brine, 20 mL×2), dried (Na₂SO₄), filtered and concentrated in vacuo to afford compound 2.545 (402 mg, 992.17 μmol, crude) as a yellow solid.

LCMS (AM12): rt=0.467 min, (302.2, [M+H]⁺), 74.38% purity.

### 5-(4-bromo-1-(tetrahydro-2H-pyran-2-yl)-1H-indazol-6-yl)-1,2,3-thiadiazole 2.546

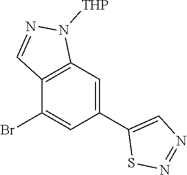

To a solution of compound 2.545 (400 mg, 987.2 μmol) in ACN (20 mL) was added t-BuONO (410.61 mg, 3.98 mmol) and CuBr (571.20 mg, 3.98 mmol). The mixture was heated to 60° C. and stirred for 0.5 h. The reaction mixture was poured into H₂O (30 mL) and extracted with EA (20 mL×3). The combined organic phases were washed (brine, 10 mL×2), dried (Na₂SO₄), filtered and concentrated in vacuo to give a residue, which was purified (PM58) to afford compound 2.546 (100 mg, 180.12 μmol, 13.57% yield) as a yellow solid.

LCMS (AM12): rt=0.606 min, (367.2, [M+2+H]⁺), 65.78% purity.

### benzyl (4-((2R)-2-((1-(tetrahydro-2H-pyran-2-yl)-6-(1,2,3-thiadiazol-5-yl)-1H-indazol-4-yl)amino)propoxy)butyl)carbamate 2.547

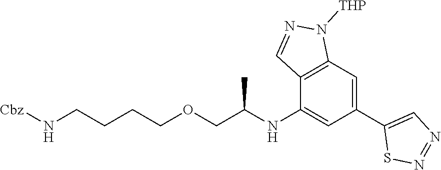

To a solution of compound 2.546 (95 mg, 260.10 μmol) and (R)-benzyl (4-(2-aminopropoxy)butyl)carbamate (Ref: WO2022185041, 218.76 mg, 780.29 μmol) in dioxane (3 mL) was added Cs₂CO₃ (254.23 mg, 780.29 μmol), Xantphos (60.20 mg, 104.04 μmol) and Pd₂(dba)₃ (47.64 mg, 52.02 μmol). The mixture was degassed and purged with N₂ (×3), then the mixture was heated to 100° C. and stirred for 12 h. The reaction mixture was cooled to 20° C., diluted with EA (20 mL) and filtered. The filtrate was concentrated in vacuo to give a residue, which was purified by prep-TLC (PM2) to afford compound 2.547 (70 mg, 115.04 μmol, 44.23% yield) as a yellow solid.

LCMS (AM12): rt=0.611 min, (565.2, [M+H]⁺), 92.85% purity.

### (R)-N-(1-(4-aminobutoxy)propan-2-yl)-6-(1,2,3-thiadiazol-5-yl)-1H-indazol-4-amine 2.548

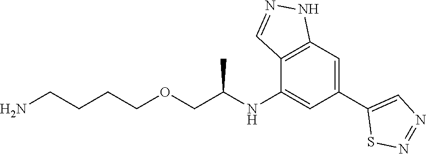 [SMILES: C[C@H](COCCCCN)Nc1cc(-c2cnns2)cc2[nH]ncc12]

A solution of compound 2.547 (65 mg, 115.11 μmol) in HCl (12 M, 8 mL) was heated to 80° C. and stirred for 0.5 h. The reaction mixture was concentrated in vacuo to afford compound 2.548 (48 mg, crude, HCl salt) as a yellow gum.

LCMS (AM12): rt=0.394 min, (347.2, [M+H]⁺), 93.14% purity.

### Synthesis of Intermediate 2.569

### methyl 4-((3-(3-(((2-chloro-[1,1′-biphenyl]-4-yl)methyl)amino)propanamido)propyl)amino)-1H-indazole-6-carboxylate 2.569

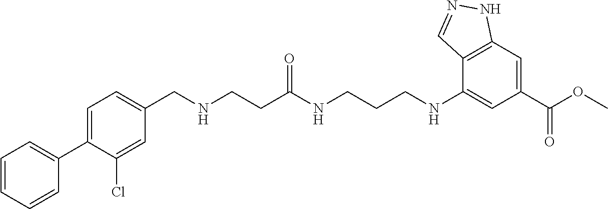 [SMILES: COC(=O)c1cc(NCCCNC(=O)CCNCc2ccc(-c3ccccc3)c(Cl)c2)c2cn[nH]c2c1]

A mixture of compound 1.81 (144 mg, 204 μmol) in HCl/MeOH (4 M, 3 mL) was stirred at 25° C. for 1 h. The solvent was removed under reduced pressure to afford compound 2.569 (100 mg, 192.30 μmol, 96.73% yield) as a yellow oil, which was used for next step directly.

LCMS (AM3): rt=0.817 min, (520.3 [M+H]⁺), 82.34% purity.

### Synthesis of Intermediate 2.574

### tert-butyl ((2-chloro-[1,1′-biphenyl]-4-yl)methyl)(4-((4,4-diethoxybutyl)amino)butyl)carbamate 2.570

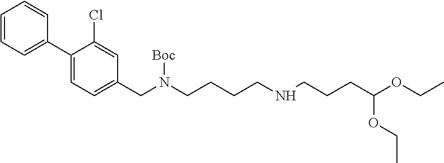 [SMILES: CCOC(CCCNCCCCN(Cc1ccc(-c2ccccc2)c(Cl)c1)C(=O)OC(C)(C)C)OCC]

To a solution of compound 1.7 (400 mg, 1.03 mmol), 4,4-diethoxybutan-1-amine (216 mg, 1.34 mmol, 231 μL) and AcOH (60.0 mg, 999 μmol) in MeOH (5 mL) was added NaBH₃CN (130 mg, 2.07 mmol) and the mixture was stirred at 30° C. for 30 min. The mixture was concentrated in vacuo to afford compound 2.570 (500 mg, crude) as a yellow oil, which was used directly for the next step.

LCMS (AM21): rt=2.441 min, (533.5 [M+H]⁺), 37.09% purity.

### tert-butyl (4-((tert-butoxycarbonyl)(4,4-diethoxybutyl)amino)butyl)((2-chloro-[1,1′-biphenyl]-4-yl)methyl)carbamate 2.571

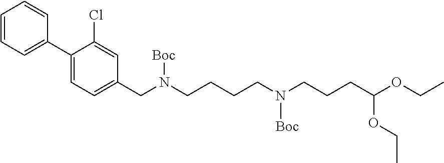 [SMILES: CCOC(CCCN(CCCCN(Cc1ccc(-c2ccccc2)c(Cl)c1)C(=O)OC(C)(C)C)C(=O)OC(C)(C)C)OCC]

To a solution of compound 2.570 (500 mg, 937.84 μmol) and TEA (189.80 mg, 1.88 mmol) in DCM (3 mL) was added Boc₂O (204.68 mg, 937.84 μmol), and the mixture was stirred at 20° C. for 1 h. The reaction mixture was poured into water (20 mL) and extracted with DCM (10 mL×3). The combined organic layer was washed (brine, 10 mL), dried (Na₂SO₄), filtered and concentrated to give a residue, which was purified (PM45) to afford compound 2.571 (350 mg, 552.70 μmol, 58.93% yield) as a yellow oil.

LCMS (AM22): rt=1.015 min, (655.5 [M+Na]⁺), 51.03% purity

### tert-butyl (4-((tert-butoxycarbonyl)(4-oxobutyl)amino)butyl)((2-chloro-[1,1′-biphenyl]-4-yl)methyl)carbamate 2.572

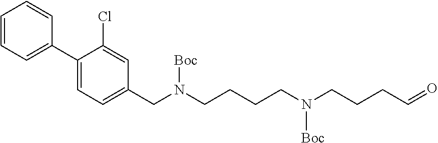 [SMILES: CC(C)(C)OC(=O)N(CCCC=O)CCCCN(Cc1ccc(-c2ccccc2)c(Cl)c1)C(=O)OC(C)(C)C]

A solution of compound 2.571 (350 mg, 553 μmol) in H₂O (5 mL) and AcOH (4.59 g, 76.5 mmol, 4.38 mL) was stirred at 50° C. for 1 h. The mixture was poured into water (30 mL) and extracted with EA (10 mL×3). The combined organic layer was washed (brine, 20 mL), dried (Na₂SO₄), filtered and concentrated to afford compound 2.572 (200 mg, 358 μmol, 64.7% yield) as a yellow oil.

LCMS (AM22): rt=0.823 min, (559.5 [M+H]⁺), 75.88% purity.

### methyl 4-((4-((tert-butoxycarbonyl)(4-((tert-butoxycarbonyl)((2-chloro-[1,1′-biphenyl]-4-yl)methyl)amino)butyl)amino)butyl)amino)-1-(tetrahydro-2H-pyran-2-yl)-1H-indazole-6-carboxylate 2.573

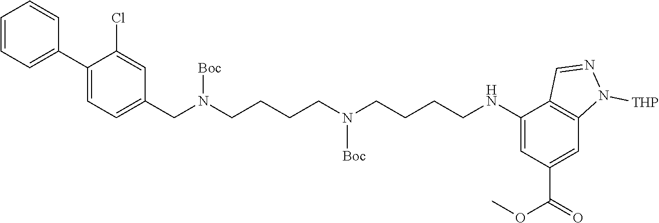

To a solution of compound 2.572 (40.0 mg, 71.5 μmol) and compound 1.80 (27.0 mg, 98.1 μmol) in MeOH (1.00 mL) was added AcOH (4.09 μL, 71.5 μmol) and the reaction was stirred at 20° C. for 0.5 h, then NaBH₃CN (20.0 mg, 318 μmol) was added to the reaction mixture and stirred at 20° C. for 2 h. The reaction mixture was concentrated under reduced pressure to give a residue, which was purified (PM183) to afford compound 2.573 (35.0 mg, crude) as a white solid.

LCMS (AM3): rt=1.278 min, (818.5 [M+H]⁺), 35.18% purity.

### methyl 4-((4-((4-(((2-chloro-[1,1′-biphenyl]-4-yl)methyl)amino)butyl)amino)butyl)amino)-1H-indazole-6-carboxylate 2.574

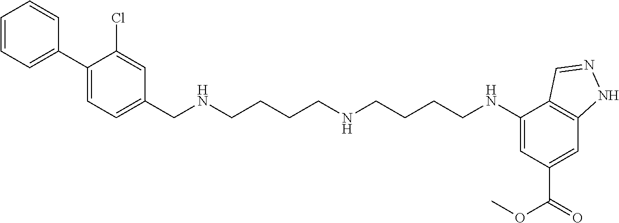 [SMILES: COC(=O)c1cc(NCCCCNCCCCNCc2ccc(-c3ccccc3)c(Cl)c2)c2cn[nH]c2c1]

To a solution of compound 2.573 (25.0 mg, 30.6 μmol) in DCM (2.00 mL) was added TFA (2.00 mL). The reaction was stirred at 30° C. for 12 h. The reaction mixture was concentrated under reduced pressure to afford compound 2.574 (16.0 mg, crude), which was used in the next step directly.

LCMS (AM3): rt=0.809 min, (534.3.5 [M+H]⁺), 93.8% purity.

### Synthesis of Intermediate 2.576

### tert-butyl (4-(2-((1-(tetrahydro-2H-pyran-2-yl)-1H-indazol-4-yl)amino)ethoxy)butyl)carbamate 2.575

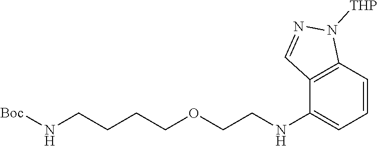

To a mixture of Pd/C (150 mg, 10% on carbon) in MeOH (30 mL) was added compound 1.893 (200 mg, 391.04 μmol) and NH₃·H₂O (146.83 mg, 1.17 mmol, 28% purity). The mixture was degassed and purged with N₂ (×3) and stirred at 20° C. for 1 h under H₂ balloon (15 psi). The mixture was filtered. The filtrate was poured into water (50 mL) and extracted with EA (50 mL×3). The combined organic phase was washed (brine, 20 mL), dried (Na₂SO₄), filtered and concentrated in vacuo to afford compound 2.575 (160 mg, 336.60 μmol, 86.08% yield) as a light-yellow solid.

LCMS (AM11): rt=0.502 min, (433.6, [M+H]⁺), 91.00% purity.

### N-(2-(4-aminobutoxy)ethyl)-1H-indazol-4-amine 2.576

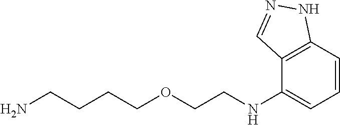 [SMILES: NCCCCOCCNc1cccc2[nH]ncc12]

To a solution of compound 2.575 (160 mg, 336.60 μmol) in MeOH (10 mL) was added HCl/dioxane (4 M, 10 mL), the mixture was stirred at 20° C. for 1 h. The mixture was concentrated in vacuo to afford compound 2.576 (130 mg, crude, HCl salt) as a yellow gum.

LCMS (AM11): rt=0.234 min, (249.4, [M+H]⁺), 87.08% purity.

### EXAMPLE COMPOUNDS

The Examples are prepared according to the methods below using the preparations hereinbefore. Wherein additional materials have been prepared, preparations are included for each Example. Alternatively, wherein commercially available materials are used, only the final steps are included, and no intermediate reference number is necessary.

### Example 1

### 3-(((2-Chloro-[1,1′-biphenyl]-4-yl)methyl)amino)-N-(3-((6-(pyridin-4-yl)-1H-indazol-4-yl)amino)propyl)propanamide

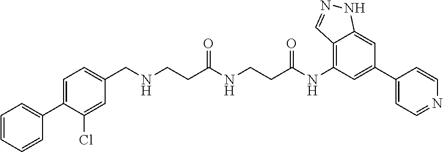 [SMILES: O=C(CCNCc1ccc(-c2ccccc2)c(Cl)c1)NCCC(=O)Nc1cc(-c2ccncc2)cc2[nH]ncc12]

A mixture of compound 1.323 (300 mg, 414.77 μmol) in aq. HCl (3 M, 30.0 mL) was stirred at 25° C. for 0.5 h. The reaction mixture was concentrated in vacuo and the residue purified (PM167) to afford EXAMPLE 1 (256.95 mg, 389.50 μmol, 93.9% yield, TFA salt) as an orange solid.

LCMS (AM3): rt=0.752 min, (539.0 [M+H]⁺), 99.4% purity.

¹H NMR (400 MHz, MeOH-d₄) δ: 8.81 (dd, J=1.2 Hz, 6.0 Hz, 2H), 8.37 (d, J=6.8 Hz, 2H), 8.25 (d, J=1.2 Hz, 1H), 7.67 (d, J=2.0 Hz, 1H), 7.50-7.35 (m, 8H), 7.33 (d, J=0.8 Hz, 1H), 4.27 (s, 2H), 3.46-3.39 (m, 4H), 3.35-3.32 (m, 2H), 2.71 (t, J=6.8 Hz, 2H), 1.98 (quin, J=6.8 Hz, 2H) ppm.

### Example 2

### 4-((3-(3-(((2-chloro-[1,1′-biphenyl]-4-yl)methyl)amino)propanamido)propyl)amino)-1H-indazole-6-carboxamide

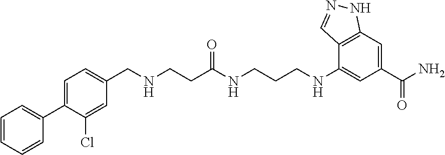 [SMILES: NC(=O)c1cc(NCCCNC(=O)CCNCc2ccc(-c3ccccc3)c(Cl)c2)c2cn[nH]c2c1]

A solution of compound 1.83 (50 mg, 0.07254 mmol) in aq. HCl (6 M, 5 mL) was stirred at 25° C. for 1 h. The reaction mixture was concentrated in vacuo and the residue was purified (PM206) to afford EXAMPLE 2 (14.94 mg, 0.0276 mmol, 38% yield, HCl salt) as a green solid.

LCMS (AM4): rt=0.781 min, (505.3 [M+H]⁺), 100% purity.

¹H NMR (400 MHz, MOH-d₄) δ 8.25-8.18 (m, 1H), 7.68 (d, J=1.6 Hz, 1H), 7.52-7.33 (m, 9H), 4.28 (s, 2H), 3.44-3.35 (m, 6H), 2.72 (t, J=6.4 Hz, 2H), 1.99-1.91 (m, 2H) ppm.

The following examples in Table 6 were made with non-critical changes or substitutions to the exemplified procedure for Example 2, that would be understood by one skilled in the art

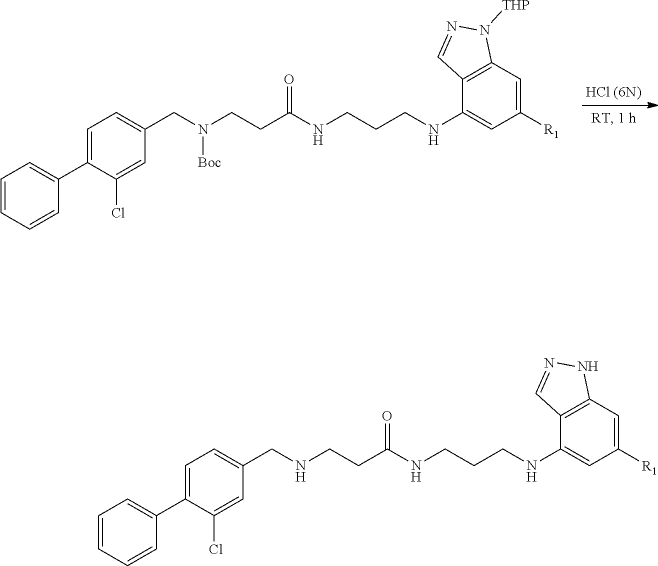

TABLE 6

| Example | Chemical | R1 structure/ | |
|---|---|---|---|
| No | IUPAC name | Intermediate No | Analytical |
| Example 3 | N-(3-((6-(1H- pyrazol-4-yl)- 1H-indazol-4- yl)amino)propyl)- | 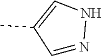 | ¹H NMR (400 MHz, MeOH-d₄) δ: 8.13 (s, 1H), 8.00 (s, 2H), 7.65 (d, J = 1.6 Hz, 1H), 7.48-7.36 (m, 9H), 4.24 (s, 2H), 3.41 (t, J = 6.8 Hz, 4H), 3.30-3.28 (m, 2H), 2.68 (t, J = |
| | 3-(((2- | Intermediate | 6.8 Hz, 2H), 2.00-1.92 (m, 2H) ppm. |
| | chloro-[1,1′- | 1.303) | LCMS (AM2): rt = 0.714 min, (528.2 |
| | biphenyl]-4- | | [M + H]⁺), 92.4% purity. |
| | yl)methyl)amino) | | Purification Method PM207 |
| | propanamide | | |
| Example 4 | N-(3-((6-(1H- 1,2,4-triazol-1- yl)-1H-indazol- 4- | 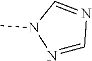 | ¹H NMR (400 MHz, MeOH-d₄) δ: 9.84 (s, 1H), 8.62 (s, 1H), 8.41 (s, 1H), 7.69 (d, J = 1.6 Hz, 1H), 7.53-7.49 (m, 1H), 7.46-7.38 (m, 8H), 4.29 (s, 2H), 3.45-3.39 (m, 4H), |
| | yl)amino)propyl)- | (Intermediate | 3.36-3.33 (m, 2H), 2.73 (t, J = 6.6 Hz, 2H), |
| | 3-(((2- | 1.295) | 2.02-1.94 (quin, 2H) ppm. |
| | chloro-[1,1′- | | LCMS (AM3): rt = 0.802 min, (529.2 |
| | biphenyl]-4- | | [M + H]⁺), 100% purity. |
| | yl)methyl)amino) | | Purification Method PM211 |
| | propanamide | | |
| Example 62 | 4-((3-(3-(((2- chloro-[1,1'- biphenyl]-4- yl)methyl)amino) propanamido) propyl)amino)- | 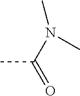 | ¹H NMR (400 MHz, MeOH-d₄) δ 8.52 (s, 1H), 7.71 (d, J = 1.6 Hz, 1H), 7.54 (dd, J = 7.6, 1.6 Hz, 1H), 7.48-7.15 (m, 8H), 4.30 (s, 2H), 3.47 (t, J = 7.2 Hz, 2H), 3.40-3.33 (m, 4H), 3.13 (s, 3H), 3.03 (s, 3H), 2.76 (t, J = 6.8 Hz, 2H), 2.03-1.94 (m, 2H) ppm |
| | N,N- | (Intermediate | LCMS (AM2): rt = 0.798 min, (533.3 |
| | dimethyl-1H- | 2.362) | [M + H]⁺), 99.57% purity. |
| | indazole-6- | | Purification Method PM212 |
| | carboxamide | | |
| Example 63 | 3-(((2-chloro- [1,1′-biphenyl]- | 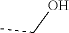 | ¹H NMR (400 MHz, MeOH-d₄) δ 8.12 (s, 1H), 7.68 (d, J = 1.6 Hz, 1H), 7.51-7.37 |
| | 4- | (Intermediate | (m, 9H), 4.65 (s, 2H), 4.27 (s, 2H), 3.41- |
| | yl)methyl)amino)- | 2.360) | 3.32 (m, 6H), 2.69 (t, J = 6.8 Hz, 2H), 1.97- |
| | N-(3-((6- | | 1.90 (m, 2H) ppm |
| | (hydroxymethyl)- | | LCMS (AM2): rt = 0.699 min, (492.2 |
| | 1H-indazol- | | [M + H]⁺), 90.45% purity |
| | 4- | | Purification Method PM207 |
| | yl)amino)propyl) | | |
| | propanamide | | |

### Example 5

### 3-((3-Chloro-4-(trifluoromethoxy)benzyl)amino)-N-(3-((6-(pyridin-4-yl)-1H-indazol-4-yl)amino)propyl)propanamide

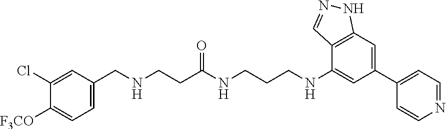 [SMILES: O=C(CCNCc1ccc(OC(F)(F)F)c(Cl)c1)NCCCNc1cc(-c2ccncc2)cc2[nH]ncc12]

A solution of compound 1.326 (240 mg, 308.79 μmol) in aq. HCl (6 M, 5 mL) was stirred at 20° C. for 0.5 h. The mixture was concentrated in vacuo and the residue was purified (PM213) to afford EXAMPLE 5 (123.48 mg, 186.81 μmol, 60.5% yield, TFA salt) as a yellow solid.

LCMS (AM3): rt=0.742 min, (547.2 [M+H]⁺), 100% purity.

¹H NMR (400 MHz, MeOH-d₄) δ: 8.80 (d, J=6.8 Hz, 2H), 8.33 (d, J=6.8 Hz, 2H), 8.24 (s, 1H), 7.77 (d, J=2 Hz, 1H), 7.57-7.51 (m, 2H), 7.32 (s, 1H), 6.56 (d, J=1.3 Hz, 1H), 4.27 (s, 2H), 3.48-3.43 (t, 2H), 3.43-3.38 (t, 2H), 3.33-3.28 (m, 2H), 2.69 (t, J=6.8 Hz, 2H), 2.03-1.94 (m, 2H) ppm.

### Example 6

### 3-(((2-chloro-[1,1′-biphenyl]-4-yl)methyl)amino)-N-(3-((6-(pyridazin-4-yl)-1H-indazol-4-yl)amino)propyl)propanamide

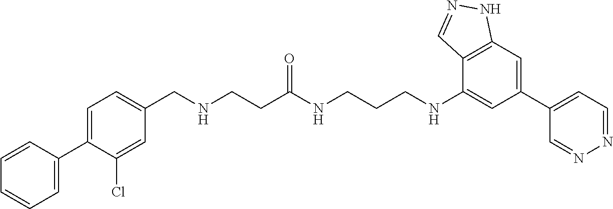 [SMILES: O=C(CCNCc1ccc(-c2ccccc2)c(Cl)c1)NCCCNc1cc(-c2ccnnc2)cc2[nH]ncc12]

To a mixture of compound 1.924 (90 mg, 124.26 μmol) in H₂O (5 mL) was added HCl (12 M, 11.66 mL) in one portion under N₂. The mixture was stirred at 25° C. for 0.5 h. The mixture was concentrated in vacuo to give a residue, which was purified (PM214) to afford EXAMPLE 6 (65.24 mg, 96.75 μmol, 77.86% yield, TFA) as a red solid.

LCMS (AM3): rt=0.771 min, (540.2 [M+H]⁺), 97.1% purity.

¹H NMR (400 MHz, DMSO-d₄) δ 9.64 (br s, 1H), 9.26 (d, J=5.2 Hz, 1H), 8.22-8.20 (m, 2H), 7.67 (s, 1H), 7.38-7.16 (m, 7H), 7.25 (s, 1H), 6.52 (s, 1H), 4.27 (s, 2H), 3.43 (q, J=6.8 Hz, 4H), 3.39-3.31 (m, 2H), 2.70 (t, J=6.8 Hz, 2H), 1.98 (t, J=6.8 Hz, 2H) ppm.

The following examples in Table 7 were made with non-critical changes or substitutions to the exemplified procedure for Example 6, that would be understood by one skilled in the art, wherein R may or may not contain a THP protecting group.

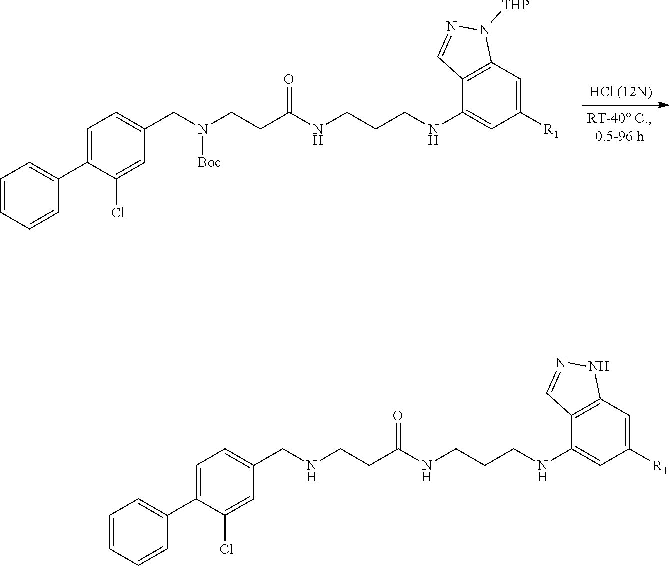

TABLE 7

| Example | Chemical | R₁ | |
|---|---|---|---|
| No. | IUPAC name | (Intermediate) | Analytical |
| Example 7 | 3-(((2-chloro- [1,1′-biphenyl]- 4- yl)methyl)amino)- N-(3-((6-(3- cyano-1H- pyrazol-4-yl)- | 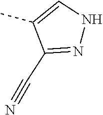 | ¹H NMR (400 MHz, DMSO-d₆) δ 8.95 (br s, 2H), 8.44 (s, 1H), 8.24-8.22 (m, 1H), 8.16 (s, 1H), 7.74 (s, 1H), 7.72-7.42 (m, 8H), 6.99 (s, 1H), 6.33 (s, 1H), 4.22 (t, J = 4.8 Hz, 2H), 3.28-3.24 (m, 4H), 3.23- 3.14 (m, 2H), 2.54 (t, J = 6.8 Hz, 2H) 1.83 (t, J = 6.8 Hz, 2H) ppm. |
| | 1H-indazol-4- | (Intermediate | LCMS (AM3): rt = 0.818 min, (553.2 |
| | yl)amino)propyl) | 1.928) | [M + H]⁺), 99.5% purity. |
| | propanamide | | Purification Method PM215 |
| Example 9 | 3-(((2-chloro- [1,1′-biphenyl]- 4- yl)methyl)amino)- N-(3-((6-(2- | 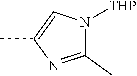 | ¹H NMR: (400 MHz, MeOH-d₄) δ: 8.19 (s, 1H), 7.76 (s, 1H), 7.69-7.68 (m, 1H), 7.49-7.40 (m, 7 H), 7.09 (m, 1H), 7.42 (s, 1H), 4.28 (s, 2H), 3.42-3.39 (m, 4H), 3.36-3.34 (m, 2H), 2.73-2.70 (m, 5H), |
| | methyl-1H- | (Intermediate | 2.01-1.94 (m, 2H) ppm. |
| | imidazol-4-yl)- | 1.918) | LCMS (AM3): rt = 0.591 min, (542.2 |
| | 1H-indazol-4- | | [M + H]⁺), 100% purity. |
| | yl)amino)propyl) | | Purification Method PM76 |
| | propanamide | | |
| Example 74 | 3-(((2-chloro- [1,1′-biphenyl]- 4- yl)methyl)amino)- N-(3-((6-(2- cyanopyridin- 4-yl)-1H- indazol-4- yl)amino)propyl) | 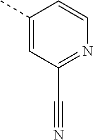 | ¹H NMR (DMSO-d₆, 400 MHz) δ 8.92- 8.76 (m, 2H), 8.75 (d, J = 5.2 Hz, 1H), 8.41 (s, 1H), 8.23 (s, 2H), 7.74 d, J = 7.2 Hz,, 1H), 7.51 (s, 1H), 7.49-7.47 (m, 7H), 7.14 (s, 1H), 6.42 (s, 1H), 4.22 (t, J = 4.8 Hz, 2H), 3.34 (t, J = 4.8 Hz, 2H), 3.28- 3.24 (m, 2H), 3.16 (t, J = 5.2 Hz, 2H), 2.54 (t, J = 6.8 Hz, 2H) 1.83 (t, J = 6.8 Hz, 2H) ppm |
| | propanamide | (Intermediate | LCMS (AM3): rt = 0.851 min, (564.3 |
| | | 2.096) | [M + H]⁺), 100.00% purity |
| | | | Purification Method PM217 |
| Example 75 | 3-(((2-chloro- [1,1′-biphenyl]- 4- yl)methyl)amino)- N-(3-((6- | 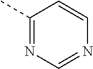 | ¹H NMR (DMSO-d₆, 400 MHz) δ 9.21 (s, 1H), 8.93 (s, 2H), 8.81 (d, J = 5.2 Hz, 1H), 8.26-8.23 (m, 2H), 7.74 (d, J = 7.2 Hz, 1H), 7.53 (s, 1H), 7.49-7.40 (m, 8H), 6.86 (s, 1H), 4.22 (t, J = 4.8 Hz, 2H), 3.34 |
| | (pyrimidin-4- | (Intermediate | (t, J = 4.8 Hz, 2H), 3.28-3.24 (m, 2H), |
| | yl)-1H-indazol- | 2.099) | 3.16 (t, J = 5.2 Hz, 2H), 2.54 (t, J = 6.8 |
| | 4- | | Hz, 2H), 1.83 (t, J = 6.8 Hz, 2H) ppm |
| | yl)amino)propyl) | | LCMS (AM3): rt = 0.825 min, (540.2 |
| | propanamide | | [M + H]⁺), 100.00% purity |
| | | | Purification Method PM208 |
| Example 76 | 3-(((2-chloro- [1,1′-biphenyl]- 4- yl)methyl)amino)- N-(3-((6-(3- methoxy-1H- pyrazol-4-yl)- | 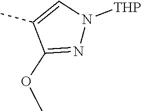 | ¹H NMR (DMSO-d₆;, 400 MHz) δ 8.98 (s, 2H), 8.35-8.25 (m, 1H), 8.10 s, 1H), 8.00 (s, 1H), 7.76 (s, 1H), 7.50-7.45 (m, 8H), 7.14 (s, 1H), 6.34 (s, 1H), 4.22 (t, J = 4.8 Hz, 2H), 3.92 (s, 3H), 3.34 (t, J = 4.8 Hz, 2H), 3.28-3.24 (m, 2H), 3.16 (t, J = 5.2 Hz, 2H), 2.54 (t, J = 6.8 Hz, 2H), 1.83 |
| | 1H-indazol-4- | (Intermediate | (t, J = 6.8 Hz, 2H) ppm |
| | yl)amino)propyl) | 2.101) | LCMS (AM3): rt = 0.827 min, (558.2 |
| | propanamide | | [M + H]⁺), 98.31% purity |
| | | | Purification Method PM214 |

### Example 8

### 3-(((2-chloro-[1,1′-biphenyl]-4-yl)methyl)amino)-N-(3-((6-(pyridin-4-yl)-1H-benzo[d][1,2,3]triazol-4-yl)amino)propyl)propanamide

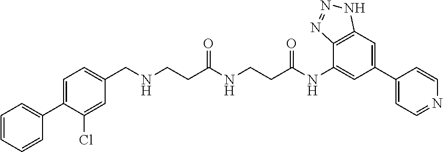 [SMILES: O=C(CCNCc1ccc(-c2ccccc2)c(Cl)c1)NCCC(=O)Nc1cc(-c2ccncc2)cc2[nH]nnc12]

To a solution of compound 1.900 (40 mg, 0.053 mmol) in H₂O (2 mL) was added HCl (2.04 g, 20.70 mmol, 37% purity). The mixture was stirred at 25° C. for 0.5 hour. The resultant mixture was then concentrated in vacuo and the residue was purified (PM216) to afford EXAMPLE 8 (28.91 mg, 92.9% yield, HCl salt) as an orange solid.

LCMS (AM3): rt=0.689 min, (540.2 [M+H]⁺), 99.2% purity.

¹H NMR: (400 MHz, CD₃OD) δ: 8.88 (d, J=6.8 Hz, 2H), 8.46 (d, J=6.8 Hz, 2H), 7.71 (s, 1H), 7.53-7.52 (m, 2H), 7.46-7.41 (m, 6H), 6.86 (s, 1H), 4.32 (s, 2H), 3.60 (t, J=6.8 Hz, 2H), 3.45 (t, J=6.8 Hz, 2H), 3.36 (t, J=6.8 Hz, 2H), 2.76 (t, J=6.8 Hz, 2H), 2.04-1.98 (m, 2H) ppm

### Example 10

### 3-(((2-chloro-[1,1′-biphenyl]-4-yl)methyl)amino)-N-(3-((6-cyano-1H-indazol-4-yl)amino)propyl)propanamide

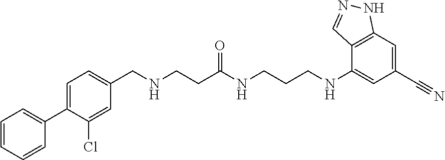 [SMILES: N#Cc1cc(NCCCNC(=O)CCNCc2ccc(-c3ccccc3)c(Cl)c2)c2cn[nH]c2c1]

To a mixture of compound 1.88 (180 mg, 0.252 mmol) in DCM (15 mL) was added TFA (4.62 g, 40.52 mmol, 3 mL) at 0° C. The reaction mixture was warmed to 25° C. and stirred for 1 h. The mixture was concentrated in vacuo and the residue was purified (PM209) to afford EXAMPLE 10 (124 mg, 0.206 mmol, 81.8% yield, TFA salt) as a yellow solid.

LCMS (AM3): rt=0.836 min, (487.2 [M+H]⁺), 100% purity.

¹H NMR (400 MHz, MeOH-d₄) δ 8.22 (d, J=0.8 Hz, 1H), 7.68 (d, J=1.6 Hz, 1H), 7.51-7.38 (m, 7H), 7.14 (s, 1H), 6.29 (s, 1H), 4.29 (s, 2H), 3.40-3.32 (m, 6H), 2.70 (t, J=6.4 Hz, 2H), 1.98-1.89 (m, 2H) ppm.

The following examples in Table 8 were made with non-critical changes or substitutions to the exemplified procedure for Example 10 that would be understood by one skilled in the art

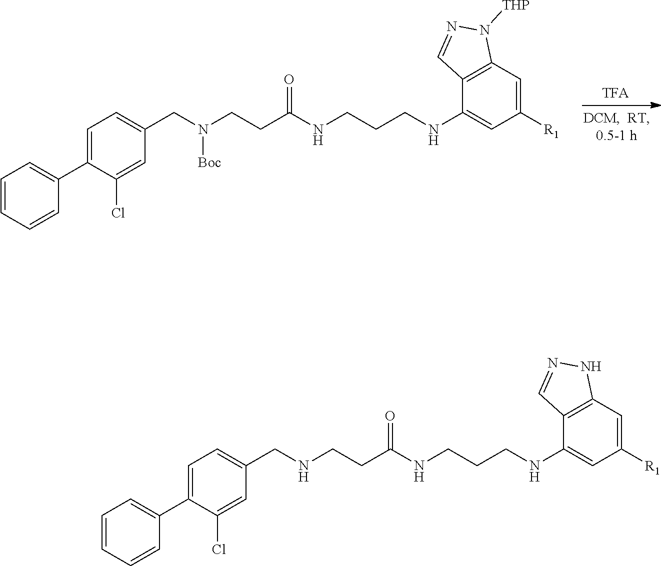

TABLE 8

| Example | Chemical | R1 | |
|---|---|---|---|
| No. | IUPAC name | (Intermediate) | Analytical |
| Example 11 | N-(3-((6-(4H- 1,2,4-triazol-4- yl)-1H-indazol- 4- | 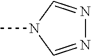 | ¹H NMR (400 MHz, DMSO-d₆) δ: 9.12 (s, 2H), 8.89 (br s, 2H), 8.24-8.18 (m, 2H), 7.74 (d, J = 1.6 Hz, 1H), 7.54-7.41 (m, 7H), 6.90 (s, 1H), 6.25 (d, J = 1.6 Hz, 1H), 4.23 (t, J = |
| | yl)amino)propyl)- | (Intermediate | 5.2 Hz, 2H), 3.31-3.25 (t, 2H), 3.25-3.20 (q, |
| | 3-(((2- | 1.274) | 2H), 3.20-3.11 (m, 2H), 2.57-2.54 (t, 2H), |
| | chloro-[1,1′- | | 1.84-1.76 (quin, 2H) ppm. |
| | biphenyl]-4- | | LCMS (AM3): rt = 0.791 min, (529.2 |
| | yl)methyl)amino) | | [M + H]⁺), 100% purity. |
| | propanamide | | Purification Method PM218 |
| Example 21 | 3-(((2-Chloro- [1,1′-biphenyl]- 4- yl)methyl)amino)- N-(3-((6-(2- methylpyridin- 4-yl)-1H- | 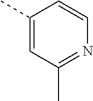 | ¹H NMR (400 MHz, MeOH-d₄) δ: 8.61 (d, J = 6.4 Hz, 1H), 8.21-8.20 (m, 2H), 8.13 (dd, J = 6.0, 1.6 Hz, 1H), 7.65 (d, J = 2.0 Hz, 1H), 7.47-7.36 (m, 7H), 7.27 (s, 1H), 6.52 (d, J = 1.2 Hz, 1H), 4.25 (s, 2H), 3.43-3.40 (m, 2H), 3.40-3.37 (m, 2H), 3.32-3.30 (m, 2H), 2.80 (s, 3H), 2.67 (d, J = 6.4 Hz, 2H), |
| | indazol-4- | (Intermediate | 1.99-1.92 (quin, 2H) ppm |
| | yl)amino)propyl) | 1.383) | LCMS (AM3): rt = 0.767 min, (553.3 |
| | propanamide | | [M + H]⁺), 99.7% purity. |
| | | | Purification Method PM220 |
| Example 22 | 3-(((2-Chloro- [1,1′-biphenyl]- 4- yl)methyl)amino)- N-(3-((6-(2- methoxypyridin- 4-yl)-1H- indazol-4- | 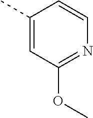 | ¹H NMR (400 MHz, MeOH-d₄) δ: 8.18 (d, J = 4.4 Hz, 2H), 7.67 (d, J = 1.6 Hz, 1H), 7.48-7.37 (m, 7H), 7.32 (dd, J = 5.2, 1.2 Hz, 1H), 7.13 (s, 1H), 7.08 (s, 1H), 6.42 (s, 1H), 4.26 (s, 2H), 4.00 (s, 3H), 3.42 (t, J = 6.8 Hz, 4H), 3.32-3.28 (m, 2H), 2.68 (t, J = 6.4 Hz, 2H), 2.01-1.94 (quin, 2H) ppm. LCMS (AM3): rt = 0.830 min, (569.3 |
| | yl)amino)propyl) | (Intermediate | [M + H]⁺), 99.4% purity. |
| | propanamide | 1.384) | Purification Method PM154 |
| Example 25 | N-(3-((6-(2H- 1,2,3-triazol-4- yl)-1H-indazol- 4- | 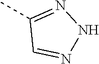 | ¹H NMR (400 MHz, DMSO-d₆) δ 8.89 (s, 2H), 8.34-8.21 (m, 2H), 8.16 (s, 1H), 7.74 (s, 1H), 7.59-7.34 (m, 8H), 7.15 (s, 1H), 6.57 (s, 1H), 4.23 (br s, 2H), 3.31-3.16 (m, |
| | yl)amino)propyl)- | (Intermediate | 6H), 2.61-2.55 (m, 2H), 1.90-1.79 (m, |
| | 3-(((2- | 1.931) | 2H) ppm |
| | chloro-[1,1′- | | LCMS (AM3): rt = 0.824 min, (529.2 |
| | biphenyl]-4- | | [M + H]⁺), 100% purity. |
| | yl)methyl)amino) | | Purification Method PM221 |
| | propanamid | | |
| Example 26 | 3-(((2-chloro- [1,1'-biphenyl]- 4- yl)methyl)amino)- N-(3-((6- | 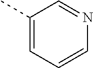 | ¹H NMR: (400 MHz, DMSO-d₆) δ: 9.04- 9.00 (m, 3H), 6.68 (d, J = 4.0 Hz, 1H), 8.37 (d, J = 8.0 Hz, 1H), 8.28-8.22 (m, 2H), 7.75-7.70 (m, 2H), 7.53-7.46 (m, 7H), 7.04 (s, 1H), 7.00 (s, 1H), 4.22 (s, 2H), 3.32 |
| | (pyridin-3-yl)- | (Intermediate | (t, J = 6.8 Hz, 2H), 3.26-3.23 (m, 2H), 3.15 |
| | 1H-indazol-4- | 1.888) | (s, 2H), 2.57 (t, J = 7.2 Hz, 2H), 1.87-1.81 |
| | yl)amino)propyl) | | (m, 2H) ppm. |
| | propanamide | | LCMS (AM3): rt = 0.767 min, (539.4 |
| | | | [M + H]⁺), 99.41% purity. |
| | | | Purification Method PM222 |
| Example 27 | 3-(((2-chloro- [1,1′-biphenyl]- 4- yl)methyl)amino)- N-(3-((6-(3- | 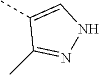 | ¹H NMR: (400 MHz, DMSO-d₆) δ: 8.95 (s, 2H), 8.27-8.24 (m, 1H), 8.12 (s, 1H), 7.79 (s, 1H), 7.75 (s, 1H), 7.55-7.41 (m, 7H), 6.69 (s, 1H), 6.13 (s, 1H), 4.22 (s, 2H), 3.27- 3.22 (m, 4H), 3.16-3.13 (m, 2H), 2.57- |
| | methyl-1H- | (Intermediate | 2.53 (m, 2H), 2.39 (s, 3H), 1.85-1.79 (m, |
| | pyrazol-4-yl)- | 1.889) | 2H) ppm. |
| | 1H-indazol-4- | | LCMS (AM3): rt = 0.787 min, (542.3 |
| | yl)amino)propyl) | | [M + H]⁺), 99.8% purity. |
| | propanamide | | Purification Method PM214 |
| Example 28 | 3-(((2-chloro- [1,1′-biphenyl]- 4- yl)methyl)amino)- | 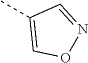 | ¹H NMR: (400 MHz, DMSO-d₆) δ: 9.43 (s, 1H), 9.17 (s, 1H), 8.92 (s, 2H), 8.25 (t, J = 6.0 Hz, 1H), 8.16 (s, 1H), 7.75 (s, 1H), 7.53- 7.42 (m, 7H), 6.97 (s, 1H), 6.32 (s, 1H), |
| | N-(3-((6- | (Intermediate | 4.23 (t, J = 5.2 Hz, 2H), 3.31-3.24 (m, 4H), |
| | (isoxazol-4-yl)- | 1.890) | 3.22-3.14 (m, 2H), 2.58-2.54 (m, 2H), |
| | 1H-indazol-4- | | 1.86-1.81 (m, 2H) ppm. |
| | yl)amino)propyl) | | LCMS (AM3): rt = 0.828 min, (529.3 |
| | propanamide | | [M + H]⁺), 99.0% purity. |
| | | | Purification Method PM223 |
| Example 29 | 3-(((2-chloro- [1,1′-biphenyl]- 4- yl)methyl)amino)- | 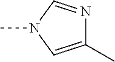 | ¹H NMR: (400 MHz, MeOH-d₄) δ: 9.28 (s, 1H), 8.22 (s, 1H), 7.76 (s, 1H), 7.66 (m, 1H), 7.43-7.37 (m, 7H), 6.95 (s, 1H), 6.29 (s, 1H), 4.25 (s, 2H), 3.38-3.34 (m, 4H), |
| | N-(3-((6-(4- | (Intermediate | 3.33-3.30 (m, 2H), 2.68 (t, J = 4.8 Hz, 2H), |
| | methyl-1H- | 1.904) | 2.42 (s, 3H), 1.96-1.89 (m, 2H) ppm |
| | imidazol-1-yl)- | | LCMS (AM3): rt = 0.780 min, (542.2 |
| | 1H-indazol-4- | | [M + H]⁺), 100% purity. |
| | yl)amino)propyl) | | Purification Method PM220 |
| | propanamide | | |
| Example 30 | 4-(4-((3-(3- (((2-chloro- [1,1′-biphenyl]- 4- yl)methyl)amino) | 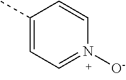 | ¹H NMR: (400 MHz, MeOH-d₄) δ: 8.39- 8.37 (m, 2H), 8.18 (s, 1H), 7.90-7.88 (m, 2H), 7.65 (s, 1 H), 7.46-7.38 (m, 8H), 7.11 (s, 1H), 4.26 (s, 2H), 3.42-3.38 (m, 4H), 3.33-3.31 (m, 2H), 2.70-2.67 (m, 2H), |
| | propanamido) | (Intermediate | 1.98-1.94 (m, 2H) ppm |
| | propyl)amino)- | 1.915) | LCMS (AM3): rt = 0.706 min, (555.1 |
| | 1H-indazol-6- | | [M + H]⁺), 97.3% purity. |
| | yl)pyridine 1- | | Purification Method PM214 |
| | oxide | | |
| Example 73 | 3-(((2-chloro- [1,1′-biphenyl]- 4- yl)methyl)amino)- N-(3-((6- | 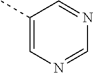 | ¹H NMR (400 MHz, MeOH-d₄) δ 9.17 (s, 1H), 9.13 (s, 2H), 8.95 (s, 2H), 8.26-8.23 (m, 1H), 8.22 (s, 1H), 7.74 (s, 1H), 7.53- 7.01 (m, 7H), 7.01 (s, 1H), 6.34 (s, 1H), 4.25-4.21 (m, 2H), 3.32 (t, J = 9.8 Hz, 2H), |
| | (pyrimidin-5- | (Intermediate | 3.26-3.23 (m, 2H), 3.23-3.16 (m, 2H), |
| | yl)-1H-indazol- | 2.265) | 2.56 (t, J = 7.2 Hz, 2H), 1.87-1.81 (m, 2H) |
| | 4- | | ppm |
| | yl)amino)propyl) | | LCMS (AM3): rt = 0.806 min, (540.1 |
| | propanamide | | [M + H]⁺), 100% purity |
| | | | Purification Method PM214 |
| Example 78 | 3-(((2-chloro- [1,1′-biphenyl]- 4- yl)methyl)amino)- N-(3-((6- | 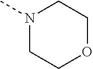 | ¹H NMR (400 MHz, MeOH-d₄) δ 8.28 (d, J = 5.6 Hz, 1H), 7.69-7.68 (m, 1H), 7.49-7.39 (m, 9H), 4.29 (s, 2H), 3.84-3.82 (m, 4H), 3.75-3.72 (m, 4H), 3.61-3.59 (m, 2H), 3.36-3.32 (m, 4H), 2.73-2.69 (m, 2H), |
| | morpholino- | (Intermediate | 2.00-1.94 (m, 2H) ppm |
| | 1H-indazol-4- | 2.268) | LCMS (AM3): rt = 0.766 min, (547.4 |
| | yl)amino)propyl) | | [M + H]⁺), 99.82% purity |
| | propanamide | | Purification Method PM231 |
| Example 79 | N-(3-((6-(2H- tetrazol-5-yl)- 1H-indazol-4- yl)amino)propyl)- 3-(((2- | 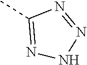 | ¹H NMR (400 MHz, MeOH-d₄) δ: 8.11 (s, 1H), 7.58 (s, 1H), 7.45-7.34 (m, 8H), 6.91 (s, 1H), 4.12 (s, 2H), 3.44-3.37 (m, 4H), 3.23-3.20 (m, 2H), 2.72-2.69 (m, 2H), 1.98-1.29 (m, 2H) ppm |
| | chloro-[1,1′- | (Intermediate | LCMS (AM3): rt = 0.807 min, (530.5 [M + |
| | biphenyl]-4- | 2.014) | H]⁺), 100% purity. |
| | yl)methyl)amino) | | Purification Method PM242 |
| | propanamide | | |
| Example 80 | 3-(((2-chloro- [1,1′-biphenyl]- 4- yl)methyl)amino)- N-(3-((6-(2- methyl-1H- | 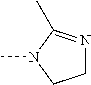 | ¹H NMR (400 MHz, MeOH-d₄) δ 8.19 (s, 1H), 7.59 (s, 1H), 7.55-7.54 (m, 1H), 7.48- 7.47 (m, 1 H), 7.35-7.34 (m, 1H), 7.33- 7.30 (m, 6H), 6.79 (s, 1H), 6.08 (s, 1H), 4.19 (s, 2H), 3.27-3.21 (m, 6H), 2.64- 2.60 (m, 2H), 2.52 (s, 3H), 1.86-1.82 (m, |
| | imidazol-1-yl)- | (Intermediate | 2H) ppm |
| | 1H-indazol-4- | 2.271) | LCMS (AM3): rt = 0.735 min, (542.2 |
| | yl)amino)propyl) | | [M + H]⁺), 99.86% purity |
| | propanamide | | Purification Method PM243 |
| Example 82 | 3-(((2-chloro- [1,1′-biphenyl]- 4- yl)methyl)amino)- | 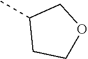 | ¹H NMR (400 MHz, MeOH-d₄) δ: 8.10 (s, 1H), 7.67 (s, 1H), 7.47-7.39 (mz, 9H), 4.26 (s, 2 H), 4.09-4.07 (m, 2H), 3.89-3.87 (m, 2H), 3.78-3.76 (m, 2H), 3.39-3.37 (m, 1H), |
| | N-(3-((6- | (Intermediate | 3.35-3.31 (m, 6H), 2.70-2.67 (m, 2H), 2.39- |
| | (tetrahydrofuran- | 2.009) | 2.36 (m, 1H), 2.08-2.05 (m, 1H), 1.94-1.90 |
| | 3-yl)-1H- | | (m, 2H) ppm |
| | indazol-4- | | LCMS (AM3): rt = 0.730 min, (532.1 |
| | yl)amino)propyl) | | [M + H]⁺), 99.6% purity. |
| | propanamide | | Purification Method PM245 |
| Example 91 | 3-(((2-chloro- [1,1′-biphenyl]- 4- yl)methyl)amino)- N-(3-((6-(4- methyl-1H- imidazol-5-yl)- | 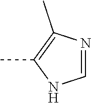 | ¹H NMR: (400 MHz, MeOH-d₄) δ: 8.83 (s, 1H), 8.20 (s, 1H), 7.67 (m, 1H), 7.47-7.46 (m, 1 H), 7.44-7.37 (m, 6H), 6.91 (s, 1H), 6.26 (s, 1H), 4.26 (s, 2H), 3.40-3.38 (m, 4H), 3.37-3.32 (m, 2H), 2.72-2.68 (m, 2H), 2.51 (s, 3H), 1.98-1.93 (m, 2H) ppm LCMS (AM3): rt = 0.611 min, (542.1 |
| | 1H-indazol-4- | (Intermediate | [M + H]⁺), 100% purity. |
| | yl)amino)propyl) | 2.006) | Purification Method PM214 |
| | propanamide | | |

### Example 12

### N-(6-(4H-1,2,4-triazol-4-yl)-1H-indazol-4-yl)-3-(3-(((2-chloro-[1,1′-biphenyl]-4-yl)methyl)amino)propanamido)propanamide

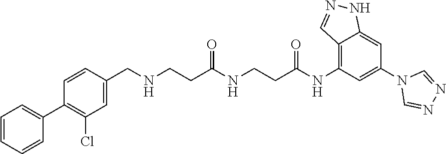 [SMILES: O=C(CCNCc1ccc(-c2ccccc2)c(Cl)c1)NCCC(=O)Nc1cc(-n2cnnc2)cc2[nH]ncc12]

To a solution of compound 1.328 (60 mg, 86.76 μmol) in DCM (2 ml-) was added TFA (0.65 ml-, 8.7 mmol) at 30° C. and the mixture was stirred for 2 h. The mixture was concentrated in vacuo and the residue was purified (PM224) to afford EXAMPLE 12 (14.76 mg, 27.18 μmol, 31.3% yield, FA salt) as a yellow solid.

LCMS (AM3): rt=0.792 min, (543.1 [M+H]⁺), 100% purity.

¹H NMR (400 MHz, MeOH-d₄) δ: 8.98 (s, 2H), 8.44 (br s, 1H), 8.37 (s, 1H), 8.00 (d, J=1.5 Hz, 1H), 7.57 (s, 1H), 7.53 (s, 1H), 7.45-7.33 (m, 7H), 4.08 (s, 2H), 3.63 (t, J=6.2 Hz, 2H), 3.19-3.13 (m, 2H), 2.78 (t, J=6.4 Hz, 2H), 2.58 (t, J=5.6 Hz, 2H) ppm.

### Example 13

### N-(3-((6-(4H-1,2,4-triazol-4-yl)-1H-indazol-4-yl)amino)propyl)-3-((3-chlorobenzyl)amino)propanamide

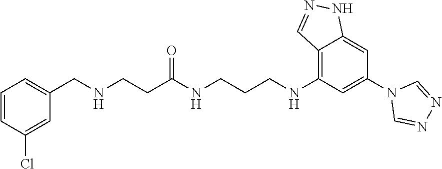 [SMILES: O=C(CCNCc1cccc(Cl)c1)NCCCNc1cc(-n2cnnc2)cc2[nH]ncc12]

To a mixture of compound 1.280 (20 mg, 0.0372 mmol) in DCM (0.5 mL) was added TFA (770 mg, 6.75 mmol) at 35° C. The mixture was stirred at 35° C. for 1 h. The mixture was concentrated in vacuo and the residue was purified (PM226) to afford EXAMPLE 13 (7.62 mg, 0.0133 mmol, 35.7% yield, TFA salt) as a yellow solid.

LCMS (AM3): rt=0.736 min, (453.1 [M+H]⁺), 99.2% purity.

¹H NMR (400 MHz, MeOH-d₄) δ: 9.02 (s, 2H), 8.20 (s, 1H), 7.56 (s, 1H), 7.48-7.42 (m, 3H), 6.93 (s, 1H), 6.27 (d, J=1.6 Hz, 1H), 4.24 (s, 2H), 3.39-3.35 (t, 4H), 3.30-3.26 (t, 2H), 2.66 (t, J=6.4 Hz, 2H), 1.98-1.91 (quin, 2H) ppm.

### Example 14

### N-(2-(4-(2-(((2-chloro-[1,1′-biphenyl]-4-yl)methyl)amino)ethyl)-1H-1,2,3-triazol-1-yl)ethyl)-6-(4H-1,2,4-triazol-4-yl)-1H-indazol-4-amine

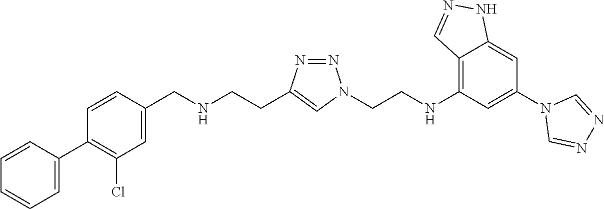 [SMILES: Clc1cc(CNCCc2cn(CCNc3cc(-n4cnnc4)cc4[nH]ncc34)nn2)ccc1-c1ccccc1]

To a solution of compound 1.320 (215 mg, 181.33 μmol) in DCM (8 mL) was added TFA (12.32 g, 108.05 mmol, 8 mL) slowly at 20° C. The reaction mixture was stirred at 20° C. for 0.5 h. The mixture was concentrated in vacuo and the crude product was purified (PM228) to afford EXAMPLE 14 (39.78 mg, 60.91 μmol, 33.6% yield, TFA salt) as a yellow solid.

LCMS (AM3): rt=0.802 min, (539.1 [M+H]⁺), 100% purity.

¹H NMR (400 MHz, MeOH-d₄) δ: 9.04 (br s, 2H), 8.12 (s, 1H), 7.84 (s, 1H), 7.67 (s, 1H), 7.47-7.37 (m, 7H), 6.98 (s, 1H), 6.21 (s, 1H), 4.71 (t, J=5.8 Hz, 2H), 4.27 (s, 2H), 3.90 (t, J=5.8 Hz, 2H), 3.31-3.28 (m, 2H), 3.06 (t, J=7.6 Hz, 2H) ppm.

### Example 15

### N-(6-(4H-1,2,4-triazol-4-yl)-1H-indazol-4-yl)-2-(2-(2-((3-chloro-4-(trifluoromethoxy)benzyl)amino)ethyl)oxazol-5-yl)acetamide

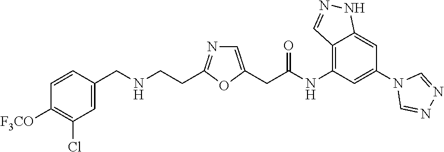 [SMILES: O=C(Cc1cnc(CCNCc2ccc(OC(F)(F)F)c(Cl)c2)o1)Nc1cc(-n2cnnc2)cc2[nH]ncc12]

To a mixture of compound 1.296 (80 mg, 107.36 μmol) in DCM (2 mL) was added TFA (3.08 g, 27.01 mmol) at 20° C. The mixture was stirred at 20° C. for 1 h. The mixture was concentrated in vacuo and the residue was purified (PM77) to afford EXAMPLE 15 (26.95 mg, 39.93 μmol, 37.2% yield, TFA salt) as a yellow solid.

LCMS (AM3): rt=0.782 min, (561.1 [M+H]⁺), 100% purity.

¹H NMR (400 MHz, MeOH-d₄) δ: 9.00 (s, 2H), 8.37 (d, J=1.2 Hz, 1H), 7.97 (d, J=1.6 Hz, 1H), 7.79 (d, J=1.6 Hz, 1H), 7.60 (s, 1H), 7.58-7.53 (m, 2H), 7.06 (s, 1H), 4.33 (s, 2H), 4.08 (s, 2H), 3.54 (t, J=6.8 Hz, 2H), 3.25 (t, J=6.8 Hz, 2H) ppm.

### Example 16

### N-(2-(5-(2-((3-chloro-4-(trifluoromethoxy)benzyl)amino)ethyl)-4H-1,2,4-triazol-3-yl)ethyl)-6-(4H-1,2,4-triazol-4-yl)-1H-indazol-4-amine

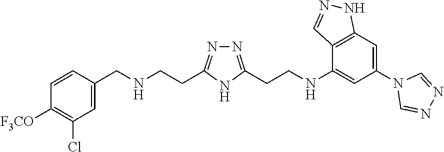 [SMILES: FC(F)(F)Oc1ccc(CNCCc2nnc(CCNc3cc(-n4cnnc4)cc4[nH]ncc34)[nH]2)cc1Cl]

To a mixture of compound 1.300 (50 mg, 68.38 μmol) in DCM (2 mL) was added TFA (3.08 g, 27.01 mmol) at 20° C. The mixture was stirred at 20° C. for 1 h. The mixture was concentrated in vacuo and the residue was purified (PM207) to afford EXAMPLE 16 (21.48 mg, 31.85 μmol, 46.6% yield, TFA salt) as a red gum.

LCMS (AM3): rt=0.772 min, (547.1 [M+H]⁺), 97.8% purity.

¹H NMR (400 MHz, MeOH-d₄) δ: 9.08 (s, 2H), 8.16 (s, 1H), 7.78 (d, J=1.6 Hz, 1H), 7.57-7.52 (m, 2H), 6.98 (s, 1H), 6.30 (d, J=1.6 Hz, 1H), 4.30 (s, 2H), 3.75 (t, J=7.0 Hz, 2H), 3.43 (t, J=8.0 Hz, 2H), 3.18 (t, J=8.0 Hz, 2H), 3.12 (t, J=7.2 Hz, 2H) ppm.

### Example 17

### N-(6-(4H-1,2,4-triazol-4-yl)-1H-indazol-4-yl)-2-((4-((3-chloro-4-(trifluoromethoxy)benzyl)amino)butyl)amino)acetamide

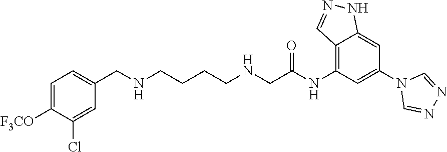 [SMILES: O=C(CNCCCCNCc1ccc(OC(F)(F)F)c(Cl)c1)Nc1cc(-n2cnnc2)cc2[nH]ncc12]

To a mixture of compound 1.289 (50 mg, 0.078 mmol) in DCM (5 mL) was added TFA (5.00 mL, 67.53 mmol) at rt for 1 h. The reaction mixture was concentrated in vacuo and the residue was purified (PM229) to afford EXAMPLE 17 (32.95 mg, 54.8% yield, 2×TFA salt) as a white solid.

LCMS (AM3): rt=0.723 min, (537.1 [M+H]⁺), 100% purity.

¹H NMR (400 MHz, MeOH-d₄) δ: 9.01 (s, 2H), 8.37 (d, J=0.8 Hz, 1H), 8.04 (d, J=1.6 Hz, 1H), 7.78 (d, J=0.8 Hz, 1H), 7.63 (s, 1H), 7.58-7.53 (m, 2H), 4.27 (s, 2H), 4.21 (s, 2H), 3.22 (t, J=7.2 Hz, 2H), 3.16 (t, J=7.2 Hz, 2H), 1.93-1.81 (m, 4H) ppm.

### Example 18

### N-(6-(4H-1,2,4-triazol-4-yl)-1H-indazol-4-yl)-3-(3-((3-chloro-4-(trifluoromethoxy)benzyl)amino)propanamido)propanamide

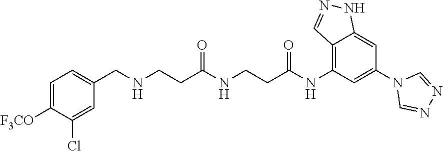 [SMILES: O=C(CCNCc1ccc(OC(F)(F)F)c(Cl)c1)NCCC(=O)Nc1cc(-n2cnnc2)cc2[nH]ncc12]

To a mixture of compound 1.286 (35 mg, 0.054 mmol) in DCM (5 mL) was added TFA (5 mL, 67.53 mmol) and the mixture stirred at rt for 1 h. The reaction mixture was concentrated in vacuo and the residue was purified (PM230) to afford EXAMPLE 18 (22.0 mg, 61.6% yield, TFA salt) as a white solid.

LCMS (AM3): rt=0.770 min, (551.1 [M+H]⁺), 100% purity.

¹H NMR (400 MHz, MeOH-d₄) δ: 9.04 (s, 2H), 8.37 (d, J=0.8 Hz, 1H), 8.01 (d, J=1.6 Hz, 1H), 7.75 (s, 1H), 7.57 (t, J=1.2 Hz, 1H), 7.53 (s, 2H), 4.26 (s, 2H), 3.63 (t, J=6.4 Hz, 2H), 3.35-3.32 (m, 2H), 2.80 (t, J=6.4 Hz, 2H), 2.67 (t, J=6.4 Hz, 2H) ppm.

### Example 19

### N-(3-((6-(4H-1,2,4-triazol-4-yl)-1H-indazol-4-yl)oxy)propyl)-3-((3-chloro-4-(trifluoromethoxy)benzyl)amino)propanamide

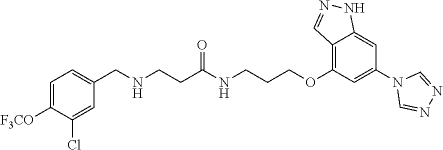 [SMILES: O=C(CCNCc1ccc(OC(F)(F)F)c(Cl)c1)NCCCOc1cc(-n2cnnc2)cc2[nH]ncc12]

To a mixture of compound 1.363 (0.3 g, 470.19 μmol) in DCM (5 mL) was added TFA (7.70 g, 67.53 mmol) at 25° C. The mixture was stirred at 25° C. for 0.5 h. The mixture was concentrated in vacuo and the residue was purified (PM231) to afford EXAMPLE 19 (113.08 mg, 171.72 μmol, 36.5% yield, TFA salt) as a white solid.

LCMS (AM3): rt=0.792 min, (538.1 [M+H]⁺), 99.1% purity.

¹H NMR (400 MHz, MeOH-d₄) δ: 9.08 (s, 2H), 8.15 (s, 1H), 7.76 (s, 1H), 7.54 (s, 2H), 7.38 (d, J=1.2 Hz, 1H), 6.81 (d, J=1.2 Hz, 1H), 4.32 (t, J=7.2 Hz, 2H), 4.26 (s, 2H), 3.49 (t, J=7.2 Hz, 2H), 3.30-3.28 (m, 2H), 2.68 (t, J=6.4 Hz, 2H), 2.17-2.07 (quin, 2H) ppm.

### Example 20

### N-(3-((6-(4H-1,2,4-triazol-4-yl)-1H-indazol-4-yl)amino)propyl)-3-((3-chloro-4-(trifluoromethoxy)benzyl)amino)-N-methylpropanamide

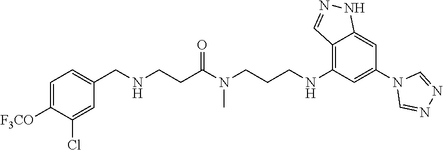 [SMILES: CN(CCCNc1cc(-n2cnnc2)cc2[nH]ncc12)C(=O)CCNCc1ccc(OC(F)(F)F)c(Cl)c1]

To a solution of compound 1.283 (70 mg, 107.51 μmol) in DCM (4 mL) was added TFA (54.02 mmol, 4 mL) at 20° C. The resulting mixture was stirred at 20° C. for 0.5 h. The mixture was concentrated in vacuo and the crude product was purified (PM232) to afford EXAMPLE 20 (68.72 mg, 102.72 μmol, 95.5% yield, TFA salt) as a brown gum.

LCMS (AM3): rt=0.716 min, (551.0 [M+H]⁺), 95.1% purity.

¹H NMR (400 MHz, DMSO-d₆) δ 8.99 (s, 2H), 8.22 (s, 1H), 7.85 (s, 1H), 7.61 (s, 2H), 6.91 (s, 1H), 6.26 (s, 1H), 4.25 (s, 2H), 3.47 (t, J=7.2 Hz, 2H), 3.31 (t, J=6.4 Hz, 2H), 3.19 (s, 2H), 3.01-2.89 (m, 3H), 2.78 (t, J=6.4 Hz, 2H), 1.93-1.93 (dd, J=2.0, 5.6 Hz, 2H) ppm

### Example 23

### N-(2-(4-(((6-chloro-1H-benzo[d]imidazol-2-yl)methyl)amino)butoxy)ethyl)-6-(4H-1,2,4-triazol-4-yl)-1H-indazol-4-amine

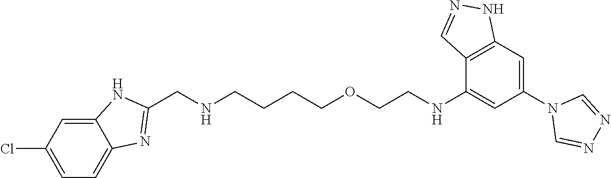 [SMILES: Clc1ccc2nc(CNCCCCOCCNc3cc(-n4cnnc4)cc4[nH]ncc34)[nH]c2c1]

To a solution of compound 1.371 (60 mg, 103.43 μmol) in DCM (10 mL) was added TFA (15.40 g, 135.06 mmol) at 20° C. The reaction mixture was stirred at 20° C. for 0.5 h. The reaction mixture was concentrated in vacuo and the crude product purified (PM232) to afford EXAMPLE 23 (21.49 mg, 35.27 μmol, 34.1% yield, TFA salt) as a brown gum.

LCMS (AM3): rt=0.732 min, (480.4 [M+H]⁺), 97.5% purity.

¹H NMR (400 MHz, MeOH-d4) δ: 9.08 (s, 2H), 8.19 (s, 1H), 7.56 (d, J=2.0 Hz, 1H), 7.52 (d, J=8.6 Hz, 1H), 7.22 (dd, J=2.0, 8.8 Hz, 1H), 6.94 (s, 1H), 6.29 (d, J=1.6 Hz, 1H), 4.44 (s, 2H), 3.79 (t, J=5.4 Hz, 2H), 3.62 (t, J=5.8 Hz, 2H), 3.54 (t, J=5.4 Hz, 2H), 3.25 (t, J=7.4 Hz, 2H), 1.92-1.85 (quin, 2H), 1.77 (quin, J=6.5 Hz, 2H) ppm.

### Example 24

### N¹-(2-((6-(4H-1,2,4-triazol-4-yl)-1H-indazol-4-yl)amino)ethyl)-N⁴-(3-chloro-4-(trifluoromethoxy)benzyl)butane-1,4-diamine

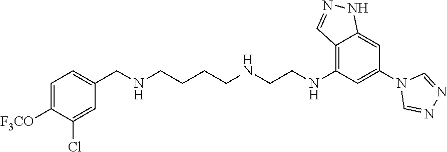 [SMILES: FC(F)(F)Oc1ccc(CNCCCCNCCNc2cc(-n3cnnc3)cc3[nH]ncc23)cc1Cl]

A mixture of compound 1.292 (80 mg, 0.109 mmol, TFA salt) in DCM (1 mL) and TFA (1 mL, 13.51 mmol) was stirred at rt for 1 h. The reaction mixture was concentrated in vacuo and the residue purified (PM232) to afford EXAMPLE 24 (50.57 mg, 62% yield, 2×TFA salt) as a white solid.

LCMS (AM3): rt=0.690 min, (523.1 [M+H]⁺), 100% purity.

¹H NMR (400 MHz, MeOH-d₄) δ: 9.03 (s, 2H), 8.19 (d, J=0.8 Hz, 1H), 7.77 (s, 1H), 7.54 (s, 2H), 7.07 (t, J=1.2 Hz, 1H), 6.41 (d, J=1.6 Hz, 1H), 4.24 (s, 2H), 3.73 (t, J=6.0 Hz, 2H), 3.38 (t, J=6.0 Hz, 2H), 3.16-3.07 (m, 4H), 1.87-1.76 (m, 4H) ppm.

### Example 31

### 3-((3-chloro-4-(trifluoromethoxy)benzyl)amino)-N-(3-((6-(2-oxo-1,2-dihydropyridin-4-yl)-1H-indazol-4-yl)amino)propyl)propanamide

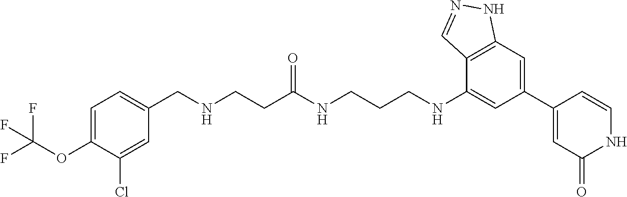 [SMILES: O=C(CCNCc1ccc(OC(F)(F)F)c(Cl)c1)NCCCNc1cc(-c2cc[nH]c(=O)c2)cc2[nH]ncc12]

To a solution of compound 1.951 (40 mg, 53.53 μmol) in DCM (2 mL) was added TFA (0.4 mL, 5.40 mmol) at 20° C. and the mixture was stirred for 0.5 h. The mixture was added into saturated NaHCO₃ aqueous solution (30 mL) and extracted with EA (20 mL×3). The combined organic phase was washed (brine, 15 mL×2), dried (Na₂SO₄), filtered and concentrated in vacuo to give a residue, which was purified (PM234) to afford EXAMPLE 31 (41.61 mg, 71.36 μmol, 63.65% yield) as a yellow solid.

LCMS (AM 16): rt=0.598 min, (563.3 [M+H]⁺), 94.12% purity.

¹H NMR (400 MHz, MeOH-d₄) δ 8.17 (s, 1H), 7.53 (s, 1H), 7.49 (d, J=7.2 Hz, 1H), 7.33-7.23 (m, 2H), 7.01 (s, 1H), 6.8-6.72 (m, 2H), 6.34 (s, 1H), 3.73 (s, 2H), 3.38 (q, J=6.8 Hz, 4H), 2.82 (t, J=6.8 Hz, 2H), 2.41 (t, J=6.8 Hz, 2H), 2.0-1.84 (m, 2H) ppm.

The following examples in Table 9 were made with non-critical changes or substitutions to the exemplified procedure for Example 31, that would be understood by one skilled in the art

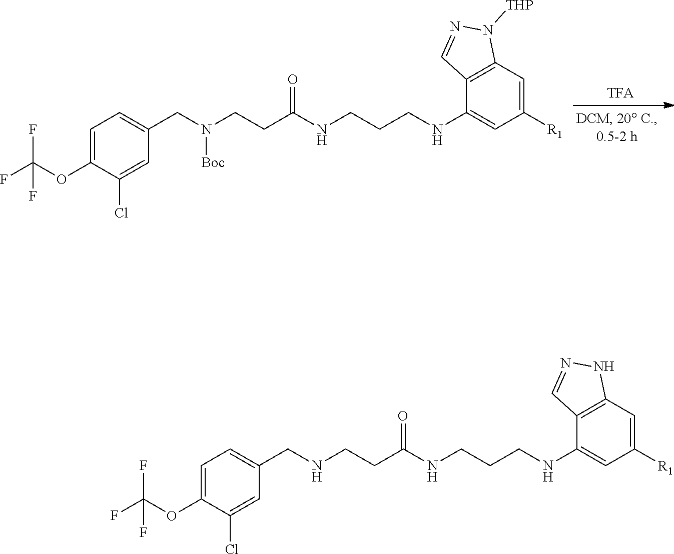

TABLE 9

| | | R1 | |
|---|---|---|---|
| Example | Chemical | structure/interme- | |
| No. | IUPAC name | diate No. | Analytical |
| Example 157 | N-(3-((6-(1,3,4- oxadiazol-2-yl)- 1H-indazol-4- yl)amino)propyl)- | 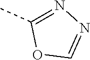 | ¹H NMR (400 MHz, MeOH-d₄) δ 9.00 (s, 1H), 8.22 (s, 1H), 7.53 (s, 1H), 7.46 (s, 1H), 7.35- 7.27 (m, 2H), 6.81 (s, 1H), 3.75 (s, 2H), 3.41-3.37 (m, 4H), 2.89 (t, J = 6.8 Hz, 2H), |
| | 3-((3-chloro-4- | (Intermediate | 2.44 (t, J = 6.8 Hz, 2H), 2.00 - 1.92 (m, 2H) |
| | (trifluoromethoxy) | 2.233) | ppm. |
| | benzyl)amino)pro- | | LCMS (AM16): rt = 0.640 min, (538.2 |
| | panamide | | [M + H]⁺), 96.15% purity. |
| | | | Purification Method PM236 |
| Example 161 | N-(3-((6-(1H- 1,2,3-triazol-1- yl)-1H-indazol-4- yl)amino)propyl)- 3-((3-chloro-4- | 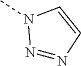 | ¹H NMR (400 MHz, MeOH-d₄) δ 8.54 (s, 1H), 8.23 (s, 1H), 7.90 (s, 1H), 7.55 (s, 1H), 7.33 (s, 2H), 7.16 (s, 1H), 6.61 (s, 1H), 3.76 (s, 2H), 3.40 (br d, J = 7.6 Hz, 4H), 2.85 (t, J = 6.8 Hz, 2H), 2.44 (t, J = 6.8 Hz, 2H), 1.982 (br |
| | (trifluoromethoxy) | (Intermediate | t, J = 6.8 Hz, 2H) ppm |
| | benzyl)amino)pro- | 2.236) | LCMS (AM16): rt = 0.640 min, (537.1 |
| | panamide | | [M + H]⁺), 99.24% purity |
| | | | Purification Method PM238 |
| Example 162 | 3-((3-chloro-4- (trifluoromethoxy) benzyl)amino)- N-(3-((6-(3- fluoropyridin-4- yl)-1H-indazol-4- yl)amino)propyl) | 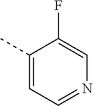 | ¹H NMR (400 MHz, MeOH-d₄) δ 8.50 (d, J = 2.8 Hz, 1H), 8.42 (d, J = 5.2 Hz, 1H), 8.16 (s, 1H), 7.64 (t, J = 5.2 Hz, 1H), 7.58 (s, 1H), 7.36 (s, 2H), 7.02 (s, 1H), 6.34 (s, 1H), 3.86 (s, 2H), 3.37 (t, J = 5.6 Hz, 4H), 2.93 (t, J = 6.8 Hz, 2H), 2.46 (t, J = 6.8 Hz, 2H), 1.96- 1.90 (m, 2H) ppm |
| | propanamide | (Intermediate | LCMS (AM11): rt = 0.341 min, (565.2 |
| | | 2.239) | [M + H]⁺), 96.89% purity |
| | | | Purification Method PM177 |
| Example 168 | 3-((3-chloro-4- (trifluoromethoxy) benzyl)amino)- N-(3-((6-(6-oxo- 1,6- dihydropyridazin- 4-yl)-1H- | 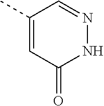 | ¹H NMR (400 MHz, MeOH-d₄) δ 8.35 (d, J = 2.0 Hz, 1H), 8.17 (s, 1H), 7.78 (d, J = 1.6 Hz, 1H), 7.57-7.52 (m, 2H), 7.16 (d, J = 2.0 Hz, 1H), 7.11 (s, 1H), 6.35 (s, 1H), 4.28 (s, 2H), 3.43-3.37 (m, 4H), 3.30-3.27 (m, 2H), 2.69 (t, J = 6.8 Hz, 2H), 2.00-1.92 (m, 2H) ppm LCMS (AM12): rt = 0.421 min, (564.1 |
| | indazol-4- | (Intermediate | [M + H]⁺), 97.03% purity |
| | yl)amino)propyl) | 2.241) | Purification Method PM246 |
| | propanamide | | |
| Example 170 | 3-((3-chloro-4- (trifluoromethoxy) benzyl)amino)- N-(3-((6-(2-oxo- 1,2- dihydropyrimidin- 4-yl)-1H- | 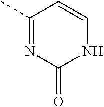 | ¹H NMR (400 MHz, MeOH-d₄) δ 8.4 (s, 1H), 8.01 (br d, J = 6.4 Hz, 1H), 7.52 (s, 1H), 7.46 (s, 1H), 7.34-7.26 (m, 2H), 7.06 (d, J = 6.4 Hz, 1H), 6.85 (s, 1H), 3.75 (s, 2H), 3.44- 3.35 (m, 4H), 2.85 (t, J = 6.8 Hz, 2H), 2.44 (t, J = 6.8 Hz, 2H), 1.97-1.90 (m, 2H) ppm LCMS (AM11): rt = 0.376 min, (564.0 |
| | indazol-4- | (Intermediate | [M + H]⁺), 94.73% purity |
| | yl)amino)propyl) | 2.247) | Purification Method PM247 |
| | propanamide | | |
| Example 174 | N-(3-((6-(4H- 1,2,4-triazol-3- yl)-1H-indazol-4- yl)amino)propyl)- | 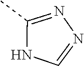 | ¹H NMR (400 MHZ, MeOH-d₄) δ 8.29 (s, 1H), 8.16 (s, 1H), 7.52 (s, 1H), 7.42 (s, 1H), 7.29 (s, 2H), 6.81 (s, 1H), 3.72 (s, 2H), 3.42- 3.36 (m, 4H), 2.83 (t, J = 6.8 Hz, 2H), 2.42 |
| | 3-((3-chloro-4- | (Intermediate | (t, J = 6.8 Hz, 2H), 1.95 (t, J = 6.8 Hz, 2H) |
| | (trifluoromethoxy) | 2.021) | ppm |
| | benzyl)amino)pro- | | LCMS (AM3) : rt = 0.779 min, (537.2 |
| | panamide | | [M + H]⁺), 96.24% purity. |
| | | | Purification Method PM249 |
| Example 177 | 3-((3-chloro-4- (trifluoromethoxy) benzyl)amino)- N-(3-((6-(3- cyano-4H-1,2,4- triazol-4-yl)-1H- indazol-4- yl)amino)propyl) | 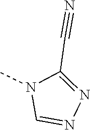 | ¹H NMR (400 MHz, MeOH-d₄) δ 9.11 (s, 1H), 8.25 (s, 1H), 7.77 (s, 1H), 7.55 (s, 2H), 6.97 (s, 1H), 6.28 (d, J = 1.2 Hz, 1H), 4.27 (s, 2H), 3.41-3.33 (m, 4H), 3.29 (m, 2H), 2.66 (t, J = 6.4 Hz, 2H), 1.95 (t, J = 7.2 Hz, 2H) ppm LCMS (AM14): rt = 0.550 min, (562.3 [M + H]⁺), 100.00% purity Purification Method SepaFlash SW040 |
| | propanamide | (Intermediate | spherical C18 20-45 μm, 100 A; [water |
| | | 2.459) | (0.1% TFA) - ACN], % B: 50%-60%, 10 min |
| Example 183 | N-(3-((6-(1,2,4- thiadiazol-5-yl)- 1H-indazol-4- yl)amino)propyl)- 3-((3-chloro-4- | 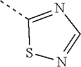 | ¹H NMR (400 MHz, MeOH-d₄) δ 8.73 (s, 1H), 8.21 (s, 1H), 7.52 (d, J = 1.6 Hz, 1H), 7.45 (s, 1H), 7.33-7.26 (m, 2H), 6.70 (d, J = 0.4 Hz, 1H), 3.74 (s, 2H), 3.39 (q, J = 6.4 Hz, 4H), 2.84 (t, J = 6.8 Hz, 2H), 2.43 (t, J = 6.8 |
| | (trifluoromethoxy) | (Intermediate | Hz, 2H), 1.96 (t, J = 6.8 Hz, 2H) ppm |
| | benzyl)amino)pro- | 2.374) | LCMS (AM14): rt = 0.573 min, (554.2 |
| | panamide | | [M + H]⁺), 100.00% purity |
| | | | Purification Method PM93 |

### Example 32

### N-(3-((6-(1,2,3-thiadiazol-5-yl)-1H-indazol-4-yl)amino)propyl)-3-((3-chloro-4-(trifluoromethoxy)benzyl)amino)propanamide

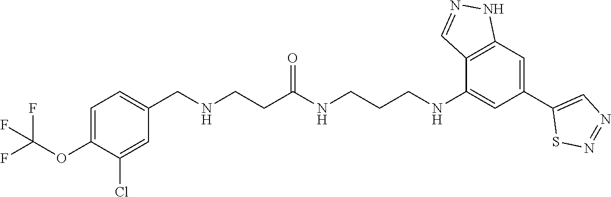 [SMILES: O=C(CCNCc1ccc(OC(F)(F)F)c(Cl)c1)NCCCNc1cc(-c2cnns2)cc2[nH]ncc12]

To a mixture of compound 1.999 (20 mg, 26.04 μmol, TFA salt) in DCM (1 mL) was added TFA (308.00 mg, 2.70 mmol, 0.2 mL) in one portion. The mixture was stirred at 25° C. for 0.5 h. The mixture was concentrated in vacuo to give a residue, which was purified (PM251) to afford EXAMPLE 32 (11.36 mg, 17.01 μmol, 65.31% yield, TFA salt) as a yellow gum.

LCMS (AM12): rt=0.473 min, (554.2 [M+H]⁺), 100% purity.

¹H NMR (400 MHz, MeOH-d₄) δ 9.12 (s, 1H), 8.18 (s, 1H), 7.76 (s, 1H), 7.53 (s, 2H), 7.15 (s, 1H), 6.39 (s, 1H), 4.26 (s, 2H), 3.40 (td, J=6.8 Hz, 2.0 Hz, 4H), 3.29 (br s, 2H), 2.68 (t, J=6.4 Hz, 2H), 1.98-1.93 (m, 2H) ppm.

¹⁹F NMR (400 MHz, MeOH-d₄) δ −59.53 (s, 3F), −77.07 (s, 6F).

The following examples in Table 10 were made with non-critical changes or substitutions to the exemplified procedure for Example 32, that would be understood by one skilled in the art

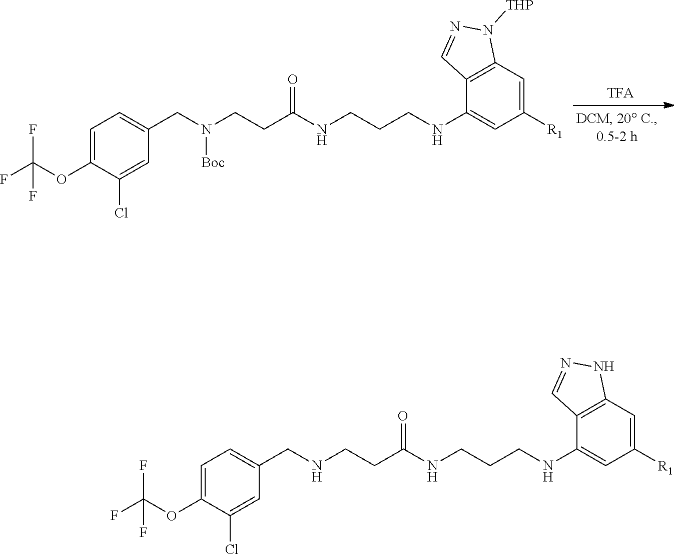

TABLE 10

| | | R1 | |
|---|---|---|---|
| Example | Chemical | structure/inter- | |
| No. | IUPAC name | mediate No. | Analytical |
| Example 160 | 3-((3-chloro-4- (trifluoromethoxy) benzyl)amino)- | 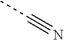 | ¹H NMR (400 MHz, MeOH-d₄) δ 8.22 (s, 1H), 7.77 (s, 1H), 7.55 (s, 2H), 7.14 (s, 1H), 6.28 (s, 1H), 4.28 (s, 2H), 3.39-3.33 (m, 6H), |
| | N-(3-((6-cyano- | (Intermediate | 2.69 (t, J = 6.4 Hz, 2H), 1.96-1.90 (m, 2H) |
| | 1H-indazol-4- | 2.253) | ppm |
| | yl)amino)propyl) | | LCMS (AM11): rt = 0.367 min, (495.1 |
| | propanamide | | [M + H]⁺), 97.4% purity |
| | | | Purification Method PM252 |
| Example 169 | 3-((3-chloro-4- (trifluoromethoxy) benzyl)amino)- N-(3-((6-(3- chloropyridin-4- yl)-1H-indazol-4- yl)amino)propyl) | 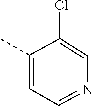 | ¹H NMR (400 MHz, MeOH-d₄) δ 8.62 (s, 1H), 8.48 (d, J = 5.2 Hz, 1H), 8.19 (s, 1H), 7.54 (s, 1H), 7.49 (d, J = 5.2 Hz, 1H), 7.32 (s, 2H), 6.82 (s, 1H), 6.18 (s, 1H), 3.73 (s, 2H), 3.32- 3.31 (m, 4H), 2.81 (t, J = 6.4 Hz, 2H), 2.39 (t, J = 6.4 Hz, 2H), 1.92 (q, J = 6.4 Hz, 2H) ppm LCMS (AM11): rt = 0.390 min, (581.1 |
| | propanamide | (Intermediate | [M + H]⁺), 98.39% purity |
| | | 2.481) | Purification Method PM256 |
| Example 187 | 3-((3-chloro-4- (trifluoromethoxy) benzyl)amino)- N-(3-((6-(6- cyanopyridazin- 4-yl)-1H- indazol-4- yl)amino)propyl) propanamide | 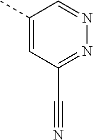 | ¹H NMR (400 MHz, MeOH-d₄) δ 9.76 (d, J = 2.0 Hz, 1H), 8.49 (d, J = 2.4 Hz, 1H), 8.21 (s, 1H), 7.53 (d, J = 2.0 Hz, 1H), 7.35-7.30 (m, 2H), 7.26 (s, 1H), 6.51 (s, 1H), 3.75 (s, 2H), 3.33-3.37 (m, 4H), 2.85 (t, J = 6.8 Hz, 2H), 2.43 (t, J = 6.8 Hz, 2H), 1.96 (t, J = 6.8 Hz, 2H) ppm LCMS (AM11): rt = 0.389 min, (573.1 [M + H]⁺), 100% purity |
| | | (Intermediate | Purification Method PM257 |
| | | 2.326) | |

### Example 33

### N-(3-((6-(4H-1,2,4-triazol-4-yl)-1H-indazol-4-yl)amino)propyl)-3-((3-chloro-4-(trifluoromethoxy)benzyl)amino)propanamide

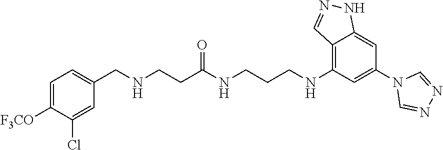 [SMILES: O=C(CCNCc1ccc(OC(F)(F)F)c(Cl)c1)NCCCNc1cc(-n2cnnc2)cc2[nH]ncc12]

A mixture of Intermediate N (50 mg, 0.137 mmol, HCl salt), NaOAc (33.7 mg, 0.411 mmol) and 3-chloro-4-(trifluoromethoxy)benzaldehyde (CAS 83279-39-4, 31 mg, 0.138 mmol) in MeOH (2 mL) was stirred at 35° C. for 16.5 h, then NaBH(AcO)₃ (116 mg, 0.548 mmol) was added. The mixture was stirred at 35° C. for 3 h. The mixture was concentrated in vacuo and the residue was purified (PM77) to afford EXAMPLE 33 (41.67 mg, 0.0640 mmol, 46.7% yield, TFA salt) as an off-white solid.

LCMS (AM3): rt=0.784 min, (537.1 [M+H]⁺), 100% purity.

¹H NMR (400 MHz, MeOH-d₄) δ: 9.09 (s, 2H), 8.20 (d, J=0.8 Hz, 1H), 7.77 (s, 1H), 7.55 (s, 2H), 6.95 (s, 1H), 6.28 (d, J=1.6 Hz, 1H), 4.28 (s, 2H), 3.38 (t, J=6.0 Hz, 4H), 3.30-3.26 (m, 2H), 2.67 (t, J=6.4 Hz, 2H), 1.98-1.91 (quin, 2H) ppm.

The following examples in Table 11 were made with non-critical changes or substitutions to the exemplified procedure for Example 33, that would be understood by one skilled in the art using Intermediate N

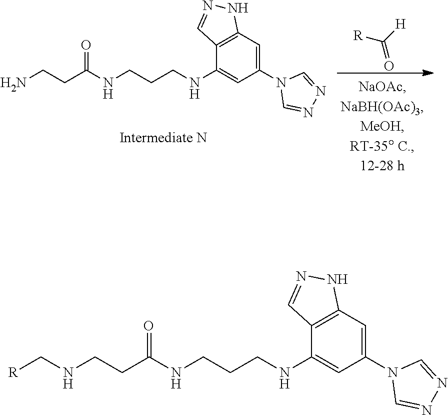

TABLE 11

| | | Aldehyde/ | |
|---|---|---|---|
| Example | Chemical | Intermediate | |
| No. | IUPAC name | No./CAS | Analytical |
| Example 34 | N-(3-((6-(4H- 1,2,4-triazol-4- yl)-1H-indazol-4- yl)amino)propyl)- 3-(((4-chloro-5- phenyl-1H- pyrazol-3- yl)methyl)amino) propanamide | 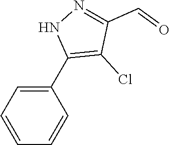 [SMILES: O=Cc1n[nH]c(-c2ccccc2)c1Cl] | ¹H NMR (400 MHz, MeOH-d4) δ: 9.04 (s, 2H), 8.21 (s, 1H), 7.74 (d, J = 1.6 Hz, 1H), 7.72 (d, J = 1.0 Hz, 1H), 7.53-7.44 (m, 3H), 6.94 (s, 1H), 6.28 (d, J = 1.6 Hz, 1H), 4.34 (s, 2H), 3.43-3.35 (m, 6H), 2.71 (t, J = 6.6 Hz, 2H), 1.95 (quin, J = 6.8 Hz, 2H) ppm. LCMS (AM3): rt = 0.755 min, (519.2 [M + H]⁺), 100% purity. Purification Method PM258 |
| | | 1.67 | |
| Example 36 | N-(3-((6-(4H- 1,2,4-triazol-4- yl)-1H-indazol-4- yl)amino)propyl)- 3-(((6-chloro-1- methyl-1H-indol- 2- yl)methyl)amino) | 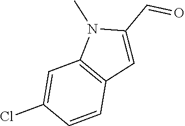 | ¹H NMR (400 MHz, MeOH-d4) δ: 9.05 (s, 2H), 8.20 (s, 1H), 7.51 (d, J = 8.4 Hz, 1H), 7.48 (s, 1H), 7.06 (dd, J = 1.8, 8.4 Hz, 1H), 6.94 (d, J = 0.7 Hz, 1H), 6.72 (s, 1H), 6.27 (d, J = 1.6 Hz, 1H), 4.52 (s, 2H), 3.80 (s, 3H), 3.37 (t, J = 6.4 Hz, 6H), 2.68 (t, J = 6.5 Hz, 2H), 1.94 (quin, J = 6.8 Hz, 2H) ppm. LCMS (AM3): rt = 0.780 min, (506.4 |
| | propanamide | CAS 1781757- | [M + H]⁺), 98.0% purity. |
| | | 70-7 | Purification Method PM207 |
| Example 37 | N-(3-((6-(4H- 1,2,4-triazol-4- yl)-1H-indazol-4- yl)amino)propyl)- 3-(((6-chloro- 1H-indol-2- yl)methyl)amino) propanamide | 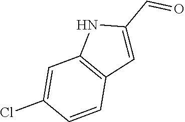 [SMILES: O=Cc1cc2ccc(Cl)cc2[nH]1] | ¹H NMR (400 MHz, MeOH-d4) δ: 9.04 (s, 2H), 8.20 (s, 1H), 7.51 (d, J = 8.6 Hz, 1H), 7.41 (s, 1H), 7.03 (dd, J = 1.8, 8.4 Hz, 1H), 6.94 (s, 1H), 6.66 (s, 1H), 6.26 (d, J = 1.6 Hz, 1H), 4.40 (s, 2H), 3.36 (t, J = 6.9 Hz, 4H), 3.32-3.28 (m, 2H), 2.65 (t, J = 6.5 Hz, 2H), 1.93 (quin, J = 6.8 Hz, 2H) ppm. LCMS (AM3): rt = 0.773 min, (492.2 |
| | | CAS 53590-59- | [M + H]⁺), 99.3 % purity. |
| | | 3 | Purification Method PM167 |
| Example 38 | N-(3-((6-(4H- 1,2,4-triazol-4- yl)-1H-indazol-4- yl)amino)propyl)- 3-(((1-methyl-5- (trifluoromethyl)- 1H-indol-2- yl)methyl)amino) propanamide | 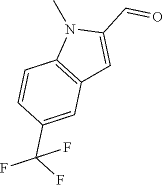 | ¹H NMR (400 MHz, MeOH-d4) δ: 9.04 (s, 2H), 8.20 (s, 1H), 7.91 (s, 1H), 7.61 (d, J = 8.4 Hz, 1H), 7.49 (dd, J = 1.6, 8.8 Hz, 1H), 6.94 (s, 1H), 6.87 (s, 1H), 6.27 (d, J = 1.2 Hz, 1H), 4.58 (s, 2H), 3.88 (s, 3H), 3.42- 3.36 (m, 6H), 2.69 (t, J = 6.4 Hz, 2H), 1.98- 1.91 (quin, 2H) ppm. LCMS (AM3): rt = 0.787 min, (540.4 [M + H]⁺), 97.8% purity. Purification Method PM259 |
| | | CAS 2090356- | |
| | | 65-1 | |
| Example 39 | N-(3-((6-(4H- 1,2,4-triazol-4- yl)-1H-indazol-4- yl)amino)propyl)- 3-((3-cyano-4- cyclobutoxybenzyl) amino)propan- | 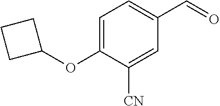 [SMILES: N#Cc1cc(C=O)ccc1OC1CCC1] | ¹H NMR (400 MHz, DMSO-d₆) δ: 9.10 (s, 2H), 8.20 (s, 1H), 7.75 (d, J = 2.4 Hz, 1H), 7.71-7.67 (m, 1H), 7.08 (d, J = 8.8 Hz, 1H), 6.95 (s, 1H), 6.28 (d, J = 1.2 Hz, 1H), 4.85- 4.82 (m, 1H), 4.20 (s, 2H), 3.39-3.35 (t, 4H), 3.26 (t, J = 6.4 Hz, 2H), 2.65 (t, J = 6.4 Hz, 2H), 2.55-2.47 (m, 2H), 2.24-2.15 (m, 2H), |
| | amide | 2-cyclobutoxy-5- | 1.97-1.86 (m, 3H), 1.83-1.71 (m, 1H) ppm. |
| | | formylbenzonitrile | LCMS (AM3): rt = 0.778 min, (514.3 |
| | | (Ref: | [M + H]⁺), 100% purity. |
| | | WO2022185041) | Purification Method PM258 |
| Example 42 | N-(3-((6-(4H- 1,2,4-triazol-4- yl)-1H-indazol-4- yl)amino)propyl)- 3-((3-(2- hydroxyethoxy)ben- zyl)amino)pro- panamide | 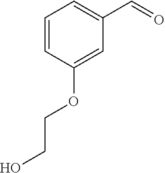 [SMILES: O=Cc1cccc(OCCO)c1] | ¹H NMR (400 MHz, MeOH-d₄) δ: 9.04 (s, 2H), 8.20 (d, J = 0.9 Hz, 1H), 7.37 (t, J = 8.0 Hz, 1H), 7.08-7.07 (m, 1H), 7.05-7.03 (m, 2H), 6.94 (d, J = 0.6 Hz, 1H), 6.28 (d, J = 1.6 Hz, 1H), 4.19 (s, 2H), 4.07 (t, J = 4.8 Hz, 2H), 3.87 (t, J = 4.8 Hz, 2H), 3.37 (t, J = 6.2 Hz, 4H), 3.27 (t, J = 6.6 Hz, 2H), 2.64 (t, J = 6.6 Hz, 2H), 1.94 (quin, J = 6.9 Hz, 2H) ppm. LCMS (AM3): rt = 0.674 min, (479.4 [M + H]⁺), 97.6 % purity. |
| | | CAS 60345-97- | Purification Method PM220 |
| | | 3 | |
| Example 43 | N-(3-((6-(4H- 1,2,4-triazol-4- yl)-1H-indazol-4- yl)amino)propyl)- 3-((3-chloro-5- (hydroxymethyl) benzyl)amino)pro- panamide | 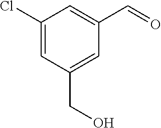 [SMILES: O=Cc1cc(Cl)cc(CO)c1] | ¹H NMR (400 MHz, MeOH-d₄) δ: 9.11 (s, 2H), 8.21 (s, 1H), 7.45 (d, J = 7.9 Hz, 2H), 7.40 (s, 1H), 6.95 (s, 1H), 6.28 (s, 1H), 4.63 (s, 2H), 4.23 (s, 2H), 3.37 (t, J = 6.5 Hz, 4H), 3.30-3.26 (m, 2H), 2.67 (t, J = 6.6 Hz, 2H), 1.94 (quin, J = 6.8 Hz, 2H) ppm. LCMS (AM3): rt = 0.696 min, (483.4 [M + H]⁺), 100% purity. |
| | | 3-chloro-5- | Purification Method PM220 |
| | | (hydroxymethyl) | |
| | | benzaldehyde | |
| | | (Ref: | |
| | | WO2022185041) | |
| Example 45 | N-(3-((6-(4H- 1,2,4-triazol-4- yl)-1H-indazol-4- yl)amino)propyl)- 3-((3-cyano-4- cyclopropylbenzyl) amino)propan- | 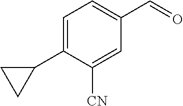 [SMILES: N#Cc1cc(C=O)ccc1C1CC1] | ¹H NMR (400 MHz, MeOH-d₄) δ: 9.00 (s, 2H), 8.20 (s, 1H), 7.78 (d, J = 2.0 Hz, 1H), 7.66 (dd, J = 2.0, 8.4 Hz, 1H), 7.12 (d, J = 8.4 Hz, 1H), 6.93 (s, 1H), 6.27 (d, J = 1.6 Hz, 1H), 4.24 (s, 2H), 3.37 (t, J = 6.8 Hz, 4H), 3.28 (t, J = 6.8 Hz, 2H), 2.67 (t, J = 6.8 Hz, 2H), 2.28-2.22 (m, 1H), 1.94 (quin, J = |
| | amide | 2-cyclopropyl-5- | 6.8 Hz, 2H), 1.21-1.16 (m, 2H), 0.87-0.84 |
| | | formylbenzonitrile | (m, 2H) ppm. |
| | | (Ref: | LCMS (AM3): rt = 0.738 min, (484.5 |
| | | WO2022185041) | [M + H]⁺), 99.2% purity. |
| | | | Purification Method PM98 |
| Example 46 | N-(3-((6-(4H- 1,2,4-triazol-4- yl)-1H-indazol-4- yl)amino)propyl)- 3-((3-cyano-4- (trifluoromethoxy) benzyl)amino) propanamide | 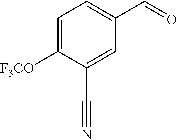 [SMILES: N#Cc1cc(C=O)ccc1OC(F)(F)F] | ¹H NMR (400 MHz, MeOH-d₄) δ: 9.03 (br s, 2H), 8.22 (br s, 1H), 8.05 (d, J = 2.4 Hz, 1H), 7.96 (dd, J = 2.4, 8.8 Hz, 1H), 7.67 (dd, J = 1.6, 8.8 Hz, 1H), 6.95 (s, 1H), 6.29 (s, 1H), 4.35 (s, 2H), 3.41-3.32 (m, 6H), 2.71 (t, J = 6.8 Hz, 2H), 1.96 (quin, J = 6.8 Hz, 2H) ppm. LCMS (AM3): rt = 0.755 min, (528.2 [M + H]⁺), 100.00% purity. |
| | | 5-Formyl-2- | Purification Method PM98 |
| | | (trifluoromethoxy) | |
| | | benzonitrile, | |
| | | (Ref: | |
| | | WO2022185041) | |
| Example 58 | N-(3-((6-(4H- 1,2,4-triazol-4- yl)-1H-indazol-4- yl)amino)propyl)- 3-(((2-chloro-2′- (hydroxymethyl)- [1,1′-biphenyl]- 4- | 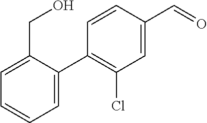 [SMILES: O=Cc1ccc(-c2ccccc2CO)c(Cl)c1] | ¹H NMR (400 MHz, MeOH-d4) δ: 9.04 (s, 2H), 8.20 (s, 1H), 7.68 (d, J = 1.6 Hz, 1H), 7.60 (d, J = 7.2 Hz, 1H), 7.50-7.48 (m, 1H), 7.46-7.43 (m, 1H), 7.39 (d, J = 7.7 Hz, 1H), 7.37-7.33 (m, 1H), 7.09 (d, J = 6.4 Hz, 1H), 6.94 (s, 1H), 6.28 (d, J = 1.5 Hz, 1H), 4.43- 4.26 (dd, 4H), 3.41-3.37 (t, 4H), 3.35-3.31 (m, 2H), 2.68 (t, J = 6.4 Hz, 2H), 1.96 (quin, |
| | yl)methyl)amino) | CAS 2830620- | J = 6.8 Hz, 2H) ppm. |
| | propanamide | 64-7 | LCMS (AM3): rt = 0.757 min, (559.2 |
| | | | [M+H]⁺), 95.7% purity. |
| | | | Purification Method PM167 |

### Example 35

### N-(3-((6-(4H-1,2,4-triazol-4-yl)-1H-indazol-4-yl)amino)propyl)-3-((3-(hydroxymethyl)benzyl)amino)propanamide

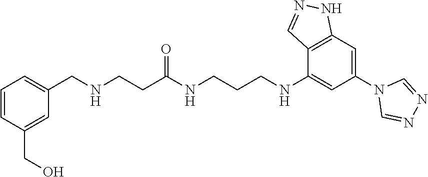 [SMILES: O=C(CCNCc1cccc(CO)c1)NCCCNc1cc(-n2cnnc2)cc2[nH]ncc12]

A mixture of 3-(hydroxymethyl) benzaldehyde (20 mg, 146.90 μmol), Intermediate N (53.59 mg, 146.90 μmol, HCl salt) and DIPEA (37.97 mg, 293.80 μmol) in MeOH (2 mL) was stirred at 25° C. for 1 h, then NaBH(ACO)₃ (155.67 mg, 734.50 μmol) was added. The mixture was stirred for 11 h. The reaction mixture was concentrated in vacuo and the residue was purified (PM260) to afford EXAMPLE 35 (51.64 mg, 89.96 μmol, 61.2% yield, TFA salt) as a purple gum.

LCMS (AM3): rt=0.648 min, (449.1 [M+H]⁺), 98.2% purity.

¹H NMR (400 MHz, MeOH-d₄) δ: 9.20 (br s, 2H), 8.22 (s, 1H), 7.47 (s, 1H), 7.44 (d, J=4.2 Hz, 2H), 7.40-7.35 (m, 1H), 6.96 (s, 1H), 6.30 (s, 1H), 4.64 (s, 2H), 4.23 (s, 2H), 3.39-3.35 (m, 4H), 3.28 (t, J=6.8 Hz, 2H), 2.67 (t, J=6.8 Hz, 2H), 1.94 (quin, J=6.8 Hz, 2H) ppm.

The following examples in Table 12 were made with non-critical changes or substitutions to the exemplified procedure for Example 35, that would be understood by one skilled in the art using Intermediate N

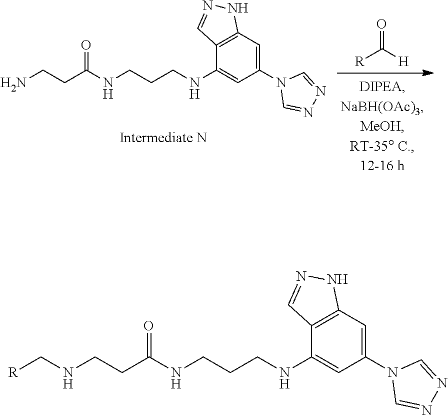

TABLE 12

| Example | Chemical | | |
|---|---|---|---|
| No. | IUPAC name | Aldehyde | Analytical |
| Example 40 | N-(3-((6-(4H- 1,2,4-triazol-4- yl)-1H-indazol- 4- yl)amino)propyl)- 3-((3-(2- hydroxyethyl)ben- zyl)amino)pro- panamide | 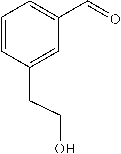 [SMILES: O=Cc1cccc(CCO)c1] | ¹H NMR (400 MHz, MeOH-d₄) δ: 9.04 (s, 2H), 8.20 (d, J = 0.8 Hz, 1H), 7.39-7.30 (m, 4H), 6.94 (d, J = 1.2 Hz, 1H), 6.27 (d, J = 1.6 Hz, 1H), 4.20 (s, 2H), 3.78 (t, J = 6.8 Hz, 2H), 3.38-3.35 (m, 4H), 3.26 (t, J = 6.8 Hz, 2H), 2.85 (t, J = 6.8 Hz, 2H), 2.64 (t, J = 6.8 Hz, 2H), 1.94 (quin, J = 6.8 Hz, 2H) ppm. LCMS (AM3): rt = 0.657 min, (463.2 [M + H]⁺), 100.00% purity. |
| | | CAS 212914- | Purification Method PM261 |
| | | 87-9 | |
| Example 41 | N-(3-((6-(4H- 1,2,4-triazol-4- yl)-1H-indazol- 4- yl)amino)propyl)- 3-((4- cyclobutoxy-3- (hydroxymethyl) | 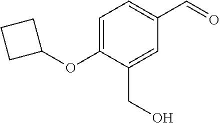 [SMILES: O=Cc1ccc(OC2CCC2)c(CO)c1] | ¹H NMR (400 MHz, MeOH-d₄) δ: 9.15 (s, 2H), 8.23 (d, J = 0.8 Hz, 1H), 7.51 (d, J = 2.4 Hz, 1H), 7.32 (dd, J = 2.4, 8.4 Hz, 1H), 6.97 (d, J = 0.8 Hz, 1H), 6.85 (d, J = 8.4 Hz, 1H), 6.31 (d, J = 1.6 Hz, 1H), 4.77-4.69 (sext, 1H), 4.66 (s, 2H), 4.18 (s, 2H), 3.41- 3.37 (m, 4H), 3.26 (t, J = 6.8 Hz, 2H), 2.66 (t, J = 6.8 Hz, 2H), 2.53-2.43 (m, 2H), 2.20- |
| | benzyl)amino) | 4-cyclobutoxy-3- | 2.09 (m, 2H), 1.98-1.91 (quin, 2H), 1.90- |
| | propanamide | (hydroxymethyl) | 1.71 (m, 2H) ppm. |
| | | benzaldehyde | LCMS (AM3): rt = 0.739 min, (519.3 |
| | | (Ref: | [M + H]⁺), 100.00% purity. |
| | | WO2022185041) | Purification Method PM262 |
| Example 47 | N-(3-((6-(4H- 1,2,4-triazol-4- yl)-1H-indazol- 4- yl)amino)propyl)- 3-((3- (cyanomethyl) benzyl)amino) propanamide | 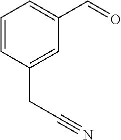 [SMILES: N#CCc1cccc(C=O)c1] | ¹H NMR (400 MHz, MeOH-d₄) δ: 9.21 (s, 2H), 8.22 (s, 1H), 7.50-7.44 (m, 4H), 6.96 (s, 1H), 6.30 (d, J = 1.2 Hz, 1H), 4.25 (s, 2H), 3.94 (s, 2H), 3.37 (t, J = 6.8 Hz, 4H), 3.32-3.27 (m, 2H), 2.68 (t, J = 6.8 Hz, 2H), 1.94 (quin, J = 6.8 Hz, 2H) ppm. LCMS (AM3): rt = 0.652 min, (458.2 [M + H]⁺), 91.5% purity. Purification Method PM263 |
| | | 2-(3- | |
| | | formylphenyl)ace- | |
| | | tonitrile (Ref: | |
| | | WO2022185041) | |

### Example 44

### N-(3-((6-(4H-1,2,4-triazol-4-yl)-1H-indazol-4-yl)amino)propyl)-3-((3-(hydroxymethyl)-4-(trifluoromethoxy)benzyl)amino)propanamide

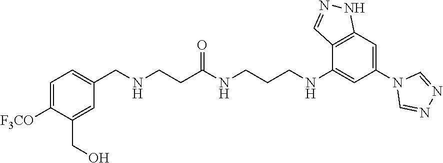 [SMILES: O=C(CCNCc1ccc(OC(F)(F)F)c(CO)c1)NCCCNc1cc(-n2cnnc2)cc2[nH]ncc12]

To a solution of Intermediate N (70 mg, 191.87 μmol, HCl salt) in MeOH (3 mL) was added DIPEA (49.60 mg, 383.74 μmol) and 3-(Hydroxymethyl)-4-(trifluoromethoxy)benzaldehyde (Ref: WO2022185041, 42.24 mg, 191.87 μmol) at 20° C. The reaction mixture was stirred at 20° C. for 12.5 h and then NaBH₃CN (36.17 mg, 575.61 μmol) was added. The reaction mixture was stirred at 20° C. for 3 h. The reaction mixture was filtered and the filtrate was purified (PM229) to afford EXAMPLE 44 (39.59 mg, 61.23 μmol, 31.9% yield, TFA salt) as a brown solid.

LCMS (AM3): rt=0.711 min, (533.4 [M+H]⁺), 100% purity.

¹H NMR (400 MHz, MeOH-d₄) δ: 9.06 (s, 2H), 8.20 (s, 1H), 7.76 (d, J=2.2 Hz, 1H), 7.50 (dd, J=2.3, 8.5 Hz, 1H), 7.37 (dd, J=1.7, 8.4 Hz, 1H), 6.95 (s, 1H), 6.28 (d, J=1.6 Hz, 1H), 4.72 (s, 2H), 4.28 (s, 2H), 3.39-3.36 (m, 4H), 3.30-3.28 (m, 2H), 2.66 (t, J=6.6 Hz, 2H), 1.94 (quin, J=6.9 Hz, 2H) ppm.

The following examples in Table 13 were made with non-critical changes or substitutions to the exemplified procedure for Example 44, that would be understood by one skilled in the art using intermediate N

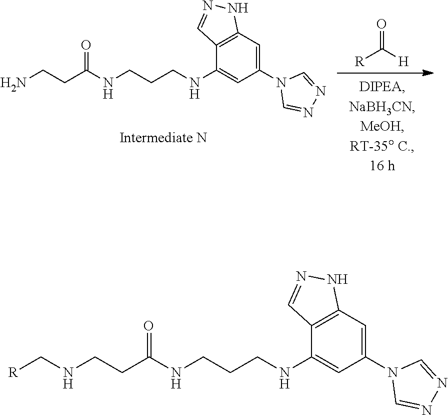

TABLE 13

| Example | Chemical | | |
|---|---|---|---|
| No. | IUPAC name | Aldehyde | Analytical |
| Example 77 | N-(3-((6-(4H- 1,2,4-triazol-4- yl)-1H-indazol- 4- yl)amino)propyl)- 3-((4- cyclopropyl-3- (hydroxymethyl) | 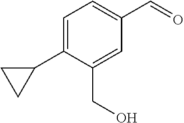 [SMILES: O=Cc1ccc(C2CC2)c(CO)c1] | ¹H NMR (400 MHz, MeOH-d₄) δ 9.00 (s, 2H), 8.20 (s, 1H), 7.51 (d, J = 1.6 Hz, 1H), 7.28 (dd, J = 8.0, 2.0 Hz, 1H), 7.06 (d, J = 8.0 Hz, 1H), 6.94 (s, 1H), 6.27 (d, J = 1.6 Hz, 1H), 4.19 (s, 2H), 3.40-3.35 (m, 4H), 3.33-3.32 (m, 2H), 3.29-3.19 (m, 2H), 2.63 (t, J = 6.4 Hz, 2H), 2.00-1.95 (m, 1H), 1.95-1.90 (m, 2H), 1.00-0.96 (m, 2H), |
| | benzyl)amino) | CAS 2830620- | 0.66-0.64 (m, 2H) ppm |
| | propanamide | 94-3 | LCMS (AM3): rt = 0.726 min, (489.2 |
| | | | [M + H]⁺), 100% purity |
| | | | Purification Method PM265 |

### Example 48

### N-(6-(4H-1,2,4-triazol-4-yl)-1H-indazol-4-yl)-2-(4-((3-chloro-4-(trifluoromethoxy)benzyl)amino)butoxy)acetamide

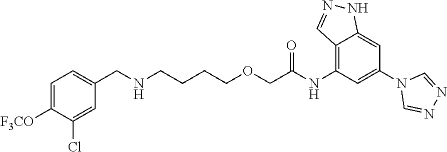 [SMILES: O=C(COCCCCNCc1ccc(OC(F)(F)F)c(Cl)c1)Nc1cc(-n2cnnc2)cc2[nH]ncc12]

A mixture of compound 1.307 (64 mg, 0.175 mmol, HCl salt), 3-chloro-4-(trifluoromethoxy)benzaldehyde (CAS 83279-39-4, 40 mg, 0.178 mmol) and DIPEA (0.07 mL, 0.402 mmol) in MeOH (10 mL) was stirred at RT for 20 h, then NaBH₃CN (44 mg, 0.7 mmol) was added. The reaction was stirred at RT for 2 h. The reaction mixture was concentrated in vacuo and the residue was purified (PM266) to afford EXAMPLE 48 (28.41 mg, 24.9% yield, TFA salt) as a yellow gum.

LCMS (AM3): rt=0.790 min, (538.1 [M+H]⁺), 100% purity.

¹H NMR (400 MHz, MeOH-d₄) δ: 9.01 (s, 2H), 8.31 (d, J=0.8 Hz, 1H), 7.94 (d, J=1.6 Hz, 1H), 7.76 (d, J=2.0 Hz, 1H), 7.61 (t, J=1.6 Hz, 1H), 7.54-7.52 (m, 1H), 7.47-7.45 (m, 1H), 4.34 (s, 2H), 4.28 (s, 2H), 3.72 (t, J=5.6 Hz, 2H), 3.21 (t, J=7.2 Hz, 2H), 1.96-1.90 (quin, 2H), 1.87-1.80 (quin, 2H) ppm.

The following examples in Table 14 were made with non-critical changes or substitutions to the exemplified procedure for Example 48, that would be understood by one skilled in the art using 3-chloro-4-(trifluoromethoxy)benzaldehyde (CAS 83279-39-4)

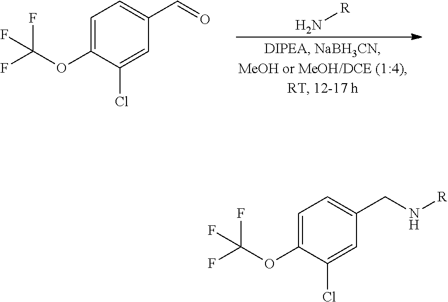

TABLE 14

| Example | Chemical IUPAC | | |
|---|---|---|---|
| No. | name | H₂NR | Analytical |
| Example 99 | N-(2-(4-((3- chloro-4- (trifluoromethoxy) benzyl)amino) butoxy)ethyl)-6- (pyridazin-4-yl)- 1H- benzo[d][1,2,3] triazol-4-amine | 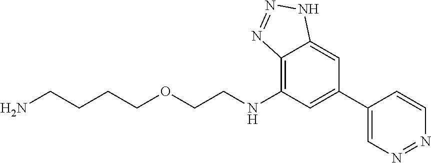 | ¹H NMR (400 MHz, MeOH-d₄) δ 9.58 (dd, J = 1.2, 2.4 Hz, 1H), 9.20 (dd, J = 1.2, 5.6 Hz, 1H), 8.04 (dd, J = 2.4, 5.6 Hz, 1H), 7.57 (s, 1H), 7.43 (d, J = 1.2 Hz, 1H), 7.36 (s, 2H), 6.73 (d, J = 1.2 Hz, 1H), 3.82 (s, 2H), 3.80 (t, J = 5.2 Hz, 2H), 3.63 (t, J = 5.2 Hz, 2H), 3.58 (br t, J = 5.6 Hz, 2H), 2.73 (br |
| | | (Intermediate 2.067) | t, J = 7.2 Hz, 2H), 1.75-1.64 |
| | | | (m, 4H) ppm |
| | | | LCMS (AM3): rt = 0.787 min, |
| | | | (536.1 [M + H]⁺), 95.6% purity |
| | | | Purification Method PM267 |
| Example 126 | N-(2-(4-((3-chloro- 4- (trifluoromethoxy) benzyl)amino) butoxy)ethyl)-6- (4H-1,2,4-triazol- 4-yl)-1H- benzo[d][1,2,3] triazol-4-amine | 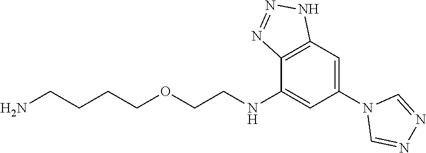 | ¹H NMR (400 MHz, MeOH-d₄) δ 9.01 (s, 2H), 7.59 (s, 1H), 7.37 (s, 2H), 7.18 (d, J = 1.6 Hz, 1H), 6.50 (d, J = 1.6 Hz, 1H), 3.82 (s, 2H), 3.78 (t, J = 5.2 Hz, 2H), 3.58 (dt, J = 5.2, 2.4 Hz, 4H), 2.72 (t, J = 7.2 Hz, 2H), 1.73-1.66 (m, 4H). LCMS (AM3): rt = 0.765 min, (525.1 [M + H]⁺), 96.7% purity |
| | | (Intermediate 2.045) | Purification Method PM268 |
| Example 127 | N-(2-(4-((3- chloro-4- (trifluoromethoxy) benzyl)amino) butoxy)ethyl)-6- (azetidine-4-yl)- 1H- benzo[d][1,2,3] triazol-4-amine | 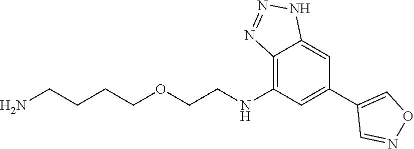 | ¹H NMR (400 MHz, MeOH-d₄) δ 9.16 (s, 1H), 8.92 (s, 1H), 8.52 (s, 1H), 7.71 (d, J = 1.6 Hz, 1H), 7.51-7.46 (m, 2H), 7.19 (d, J = 1.2 Hz, 1H), 6.68 (d, J = 1.2 Hz, 1H), 4.15 (s, 2H), 3.84-3.80 (m, 2H), 3.67- 3.60 (m, 4H), 3.13-3.06 (m, 2H), 1.92-1.82 (m, 2H), 1.79- |
| | | (Intermediate 2.064) | 1.70 (m, 2H) ppm |
| | | | LCMS (AM3): rt = 0.793 min, |
| | | | (525.1 [M + H]⁺), 99.57% purity |
| | | | Purification Method PM269 |
| | | | then 2) PM270 |
| Example 150 | N-(3-chloro-4- (trifluoromethoxy) benzyl)-4-((1-(6- (pyridazin-4-yl)- 1H-benzo [d][1,2,3]triazol- 4-yl)azetidin- 3-yl)oxy)butan-1- amine | 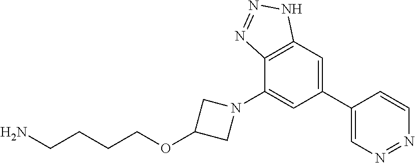 | ¹H NMR (400 MHz, MeOH-d₄) δ 9.57 (dd, J = 1.2, 2.4 Hz, 1H), 9.20 (dd, J = 1.2, 5.6 Hz, 1H), 8.04 (dd, J = 2.4, 5.6 Hz, 1H), 7.58 (d, J = 0.8 Hz, 1H), 7.45 (s, 1H), 7.37 (s, 2H), 6.48 (s, 1H), 4.64-4.59 (m, 2H), 4.59-4.54 (m, 1H), 4.17 (dd, J = 3.8, 8.9 Hz, 2H), 3.83 (s, 2H), 3.61-3.53 (m, 2H), 2.75- |
| | | (Intermediate 2.285) | 2.65 (m, 2H), 1.70-1.66 (m, |
| | | | 4H) ppm |
| | | | LCMS (AM3): rt = 0.775 min, |
| | | | (548.1 [M + H]⁺), 98.00% purity |
| | | | Purification method 1) |
| | | | PM271 then 2) PM272 |

### Example 49

### 6-(4H-1,2,4-triazol-4-yl)-N-(2-(4-(((5-(trifluoromethyl)-1H-indol-2-yl)methyl)amino)butoxy)ethyl)-1H-indazol-4-amine

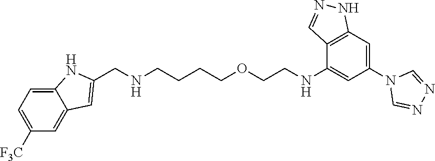 [SMILES: FC(F)(F)c1ccc2[nH]c(CNCCCCOCCNc3cc(-n4cnnc4)cc4[nH]ncc34)cc2c1]

A solution of compound 1.306 (80 mg, 227.38 μmol, HCl salt), DIPEA (58.77 mg, 454.76 μmol) and 5-(trifluoromethyl)-1H-indole-2-carbaldehyde (48.47 mg, 227.38 μmol) in MeOH (10 mL) was stirred at 30° C. for 3.5 h, then NaBH₃CN (42.87 mg, 682.14 μmol) was added. The reaction mixture was stirred at 30° C. for 3 h. The reaction mixture was filtered and the filtrate was purified (PM273) to afford EXAMPLE 49 (30.39 mg, 48.07 μmol, 21% yield, TFA salt) as a white solid.

LCMS (AM3): rt=0.800 min, (513.4 [M+H]⁺), 99.1% purity.

¹H NMR (400 MHz, MeOH-d4) δ: 9.01 (s, 2H), 8.19 (s, 1H), 7.88 (s, 1H), 7.54 (d, J=8.4 Hz, 1H), 7.41 (dd, J=1.6, 8.8 Hz, 1H), 6.95 (s, 1H), 6.74 (s, 1H), 6.33 (d, J=1.6 Hz, 1H), 4.34 (s, 2H), 3.74 (t, J=5.6 Hz, 2H), 3.58 (t, J=6.0 Hz, 2H), 3.52 (t, J=5.4 Hz, 2H), 3.11 (t, J=7.6 Hz, 2H), 1.86-1.78 (quin, 2H), 1.74-1.67 (quin, 2H) ppm.

The following examples in Table 15 were made with non-critical changes or substitutions to the exemplified procedure for Example 49, that would be understood by one skilled in the art using intermediate 1.306

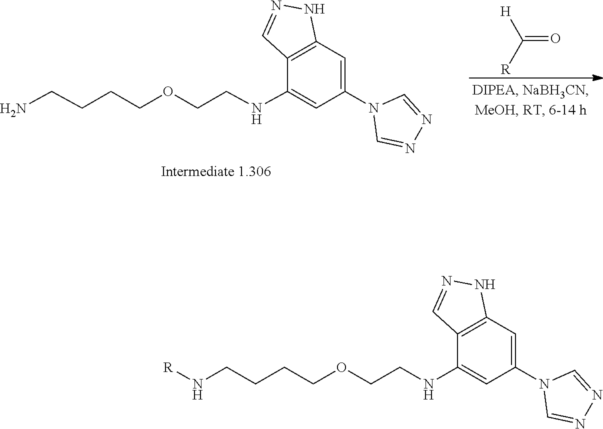

TABLE 15

| | | Aldehyde | |
|---|---|---|---|
| | | (RCHO) | |
| Example | Chemical | Intermediate | |
| No. | IUPAC name | No./CAS | Analytical |
| Example 50 | N-(2-(4-(((6- chloro-1- methyl-1H- indol-2- yl)methyl)amino) butoxy)ethyl)- 6-(4H-1,2,4- triazol-4-yl)- 1H-indazol-4- amine | 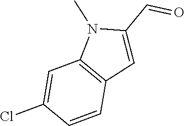 CAS 1781757-70-7 | ¹H NMR (400 MHz, MeOH-d4) δ: 9.01 (s, 2H), 8.20 (s, 1H), 7.49-7.45 (m, 2H), 7.04 (dd, J = 1.6, 8.4 Hz, 1H), 6.93 (s, 1H), 6.64 (s, 1H), 6.32 (s, 1H), 4.38 (s, 2H), 3.76 (t, J = 5.6 Hz, 2H), 3.72 (s, 3H), 3.60 (t, J = 5.8 Hz, 2H), 3.52 (t, J = 5.6 Hz, 2H), 3.16 (t, J = 7.6 Hz, 2H), 1.87-1.80 (quin, 2H), 1.76-1.89 (quin, 2H) ppm. LCMS (AM3): rt = 0.786 min, (493.3 [M + H]⁺), 99.5% purity. Purification Method PM230 |
| Example 51 | N-(2-(4-(((1- methyl-5- (trifluoromethyl)- 1H-indol-2- yl)methyl)amino) butoxy)ethyl)- 6-(4H-1,2,4- triazol-4-yl)- 1H-indazol-4- amine | 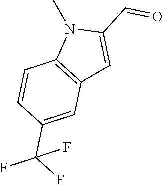 CAS 2090356-65-1 | ¹H NMR (400 MHz, MeOH-d4) δ: 9.04 (s, 2H), 8.21 (s, 1H), 7.87 (s, 1H), 7.57 (d, J = 8.6 Hz, 1H), 7.46 (dd, J = 1.6, 8.8 Hz, 1H), 6.94 (s, 1H), 6.79 (s, 1H), 6.32 (s, 1H), 4.44 (s, 2H), 3.81 (s, 3H), 3.77 (t, J = 5.2 Hz, 2H), 3.60 (t, J = 6.0 Hz, 2H), 3.53 (t, J = 5.6 Hz, 2H), 3.18 (t, J = 8.0 Hz, 2H), 1.89-1.82 (quin, 2H), 1.78-1.70 (quin, 2H) ppm. LCMS (AM3): rt = 0.802 min, (527.4 [M + H]⁺), 99.6% purity. Purification Method PM255 |
| Example 52 | N-(2-(4-(((6- chloro-1H- indol-2- yl)methyl)amino) butoxy)ethyl)- 6-(4H-1,2,4- triazol-4-yl)- 1H-indazol-4- amine | 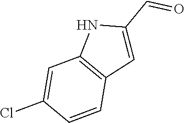 CAS 53590-59-3 | ¹H NMR (400 MHz, MeOH-d4) δ: 8.98 (s, 2H), 8.20 (s, 1H), 7.38 (d, J = 8.4 Hz, 1H), 7.29 (s, 1H), 6.94-6.91 (m, 2H), 6.33-6.32 (m, 2H), 3.85 (s, 2H), 3.71 (t, J = 5.6 Hz, 2H), 3.53-3.49 (m, 4H), 2.62 (t, J = 6.8 Hz, 2H), 1.65-1.56 (m, 4H) ppm. LCMS (AM3): rt = 0.777 min, (479.4 [M + H]⁺), 100% purity. Purification Method PM62 |

### Example 53

### N-(2-(4-(((5-cyclobutoxy-1H-indol-2-yl)methyl)amino)butoxy)ethyl)-6-(4H-1,2,4-triazol-4-yl)-1H-indazol-4-amine

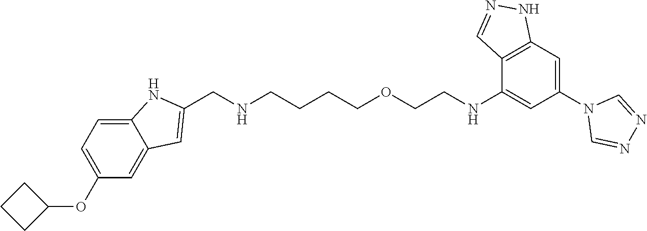 [SMILES: c1cc2[nH]c(CNCCCCOCCNc3cc(-n4cnnc4)cc4[nH]ncc34)cc2cc1OC1CCC1]

A mixture of compound 1.420 (110 mg, 0.511 mmol), compound 1.306 (200 mg, 0.515 mmol, HCl salt) and DIPEA (1.55 mmol, 0.27 mL) in MeOH (10 mL) was stirred at 16° C. for 15 h before NaBH(OAc)₃ (0.44 g, 2.08 mmol) was added, then the reaction mixture was stirred for 2 h. The reaction mixture was concentrated in vacuo to give a residue, which was purified (PM274) then (PM134) to afford EXAMPLE 53 (79.97 mg, 30.41% yield) as an off-white solid.

LCMS (AM3): rt=0.806 min, (515.5 [M+H]⁺), 100% purity.

¹H NMR: (400 MHz, MeOH-d₄) δ 8.96 (s, 2H), 8.20 (d, J=1.2 Hz, 1H), 7.18 (d, J=8.8 Hz, 1H), 6.92 (s, 1H), 6.85 (d, J=2.4 Hz, 1H), 6.67 (d, J=8.8 Hz, 2.4 Hz, 1H), 6.32-6.28 (m, 2H), 4.64-4.54 (m, 1H), 3.96 (s, 2H), 3.70 (t, J=5.6 Hz, 2H), 3.56-3.45 (m, 4H), 2.74 (t, J=6.8 Hz, 2H), 2.47-2.36 (m, 2H), 2.16-2.02 (m, 2H), 1.87-1.76 (m, 1H), 1.73-1.67 (m, 1H), 1.67-1.60 (m, 4H) ppm.

The following examples in Table 16 were made with non-critical changes or substitutions to the exemplified procedure for Example 53, that would be understood by one skilled in the art using Intermediate 1.420

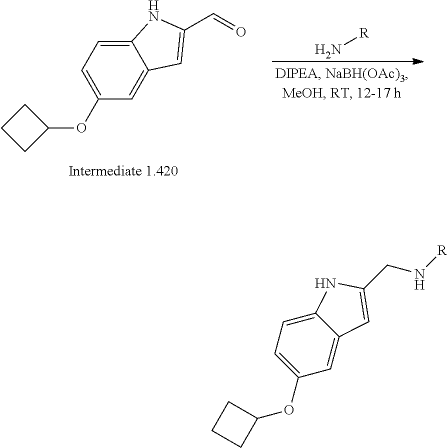

TABLE 16

| Example | Chemical IUPAC | | |
|---|---|---|---|
| No. | name | H₂NR | Analytical |
| Example 54 | N-((5-cyclobutoxy-1H- indol-2-yl)methyl)- 4-(2-((6-(isoxazol- 4-yl)-1H-indazol-4- yl)oxy)ethoxy)butan-1-amine | 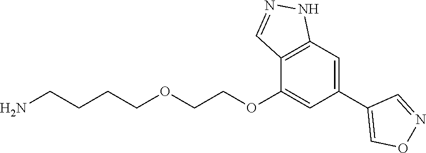 (Intermediate 1.910) | ¹H NMR: (400 MHz, MeOH-d₄) δ: 9.13 (s, 1H), 8.90 (s, 1H), 8.56 (s, 1H), 8.06 (s, 1H), 7.36 (s, 1H), 7.23-7.21 (m, 1H), 6.87-6.86 (m, 1H), 7.78 (s, 1H), 6.74-6.71 (m, 1H), 6.40 (s, 1H), 4.61-4.60 (m, 1H), 4.34-4.32 (m, 2H), 4.16 (s, 2H), 3.90-3.88 (m, 2H), 3.65- 3.63 (m, 2H), 3.30-2.97 (m, 2H), 2.41-2.40 (m, 2H), 2.10- 2.12 (m, 2H), 1.80-1.69 (m, |
| | | | 6H) ppm. |
| | | | LCMS (AM3): rt = 0.874 min, |
| | | | (516.5 [M + H]⁺), 100% purity. |
| | | | Purification Method PM275 |
| Example 55 | N-((5-cyclobutoxy-1H- indol-2-yl)methyl)- 4-(2-((6-(pyridazin-4-yl)- 1H-indazol-4- yl)oxy)ethoxy) butan-1-amine | 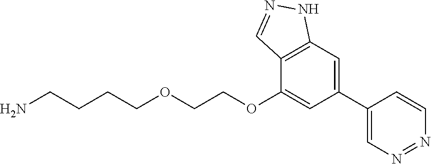 (Intermediate 1.913) | ¹H NMR: (400 MHz, MeOH-d₄) δ: 9.58-9.57 (m, 1H), 9.20- 9.19 (m, 1H), 8.15-8.14 (m, 1H), 8.02-8.00 (m, 1H), 7.56 (s, 1H), 7.15-7.13 (m, 1H), 6.92 (d, J = 0.8 Hz, 1H), 6.82 (d, J = 2.8 Hz, 1H), 6.64-6.61 (m, 1H), 6.20 (s, 1H), 4.60- 4.58 (m, 1H), 4.39-4.37 (m, 2H), 3.90-3.87 (m, 2H), 3.82 (s, 2H), 3.60-3.58 (m, 2H), 2.65-2.63 (m, 2H), 2.41-2.39 |
| | | | (m, 2H), 2.14-2.04 (m, 2H), |
| | | | 1.85-1.75 (m, 1H), 1.68-1.57 |
| | | | (m, 5H) ppm. |
| | | | LCMS (AM3): rt = 0.781 min, |
| | | | (527.1 [M + H]⁺), 97.1% purity. |
| | | | Purification Method PM276 |
| Example 98 | N-(2-(4-(((5- cyclobutoxy-1H- indol-2-yl)methyl)amino) butoxy)ethyl)-6- (isoxazol-4-yl)-1H- indazol-4-amine | 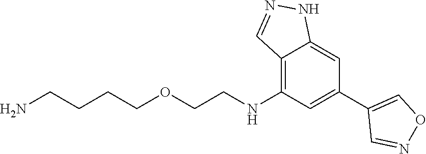 (Intermediate 1.895) | ¹H NMR (400 MHz, MeOH-d₄) δ 9.04 (s, 1H), 8.82 (s, 1H), 8.11 (s, 1H), 7.16 (d, J = 8.4 Hz, 1H), 6.99 (s, 1H), 6.87- 6.80 (m, 1H), 6.69-6.62 (m, 1H), 6.39 (s, 1H), 6.23 (s, 1H), 4.65-4.55 (m, 1H), 3.84 (s, 2H), 3.70 (t, J = 5.6 Hz, 2H), 3.52-3.46 (m, 4H), 2.67-2.58 (m, 2H), 2.55-2.46 (m, 2H), 2.13-2.05 (m, 2H), 1.92-1.85 (m, 1H), 1.71-1.65 (m, 1H), |
| | | | 1.46-1.39 (m, 4H) ppm |
| | | | LCMS (AM3): rt = 0.851 min, |
| | | | (515.3 [M + H]⁺), 100% purity |
| | | | Purification Method PM278 |
| Example 109 | N-((5- cyclobutoxy-1H-indol-2- yl)methyl)-4-((1-(6- (pyridazin-4-yl)-1H- indazol-4-yl)azetidin-3- yl)oxy)butan-1-amine | 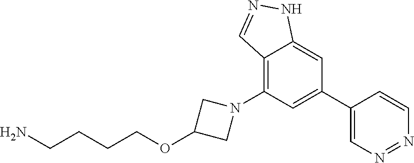 (Intermediate 2.113) | ¹H NMR (400 MHz, MeOH-d₄) δ 9.55 (d, J = 1.2, 2.4 Hz, 1H), 9.19 (dd, J = 1.2, 5.6 Hz, 1H), 8.06 (d, J = 0.8 Hz, 1H), 8.01 (dd, J = 2.4, 5.6 Hz, 1H), 7.27 (s, 1H), 7.16 (d, J = 8.8 Hz, 1H), 6.85 (d, J = 2.4 Hz, 1H), 6.64 (dd, J = 2.4, 8.8 Hz, 1H), 6.29-6.21 (m, 2H), 4.60 (d, J = 7.6 Hz, 1H), 4.50-4.45 (m, 1H), 4.45-4.40 (m, 2H), 4.01 (dd, J = 3.6, 8.4 Hz, 1H), |
| | | | 3.87 (s, 2H), 3.53-3.46 (m, |
| | | | 2H), 2.65 (t, J = 6.8 Hz, 2H), |
| | | | 2.46-2.37 (m, 2H), 2.15- |
| | | | 2.03 (m, 2H), 1.86-1.74 (m, |
| | | | 1H), 1.73-1.66 (m, 1H), 1.66- |
| | | | 1.59 (m, 4H) ppm |
| | | | LCMS (AM3): rt = 0.819 min, |
| | | | 538.3 [M + H]⁺), 100.00% |
| | | | purity |
| | | | Purification method 1) |
| | | | PM279 then 2) PM280 |
| Example 118 | 4-(2-((6-(4H- 1,2,4-triazol-4-yl)-1H- indazol-4-yl)oxy)ethoxy)-N- ((5-cyclobutoxy-1H-indol- 2-yl)methyl)butan-1-amine | 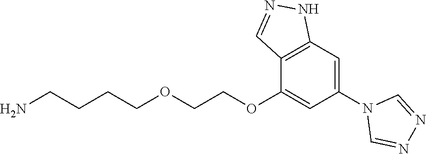 (Intermediate 1.374) | ¹H NMR (400 MHz, MeOH-d₄) δ 9.02 (s, 2H), 8.16 (s, 1H), 7.35 (s, 1H), 7.14 (d, J = 8.8 Hz, 1H), 6.83 (d, J = 2.4 Hz, 1H), 6.79 (s, 1H), 6.65-6.60 (m, 1H), 6.20 (s, 1H), 4.62- 4.57 (m, 1H), 4.36-4.32 (m, 2H), 3.89-3.85 (m, 2H), 3.83 (s, 2H), 3.62-3.57 (m, 2H), 2.64 (t, J = 7.2 Hz, 2H), 2.46- 2.38 (m, 2H), 2.15-2.05 (m, 2H), 1.85-1.77 (m, 1H), 1.74- |
| | | | 1.67 (m, 1H), 1.67-1.61 (m, |
| | | | 4H) ppm |
| | | | LCMS (AM3): rt = 0.822 min, |
| | | | (516.1 [M + H]⁺), 100% purity |
| | | | Purification Method PM282 |
| Example 119 | N-((5-cyclobutoxy-1H- indol-2-yl)methyl)- 4-((1-(6-(isoxazol- 4-yl)-1H-indazol-4-yl) azetidin-3-yl)oxy)butan- 1-amine | 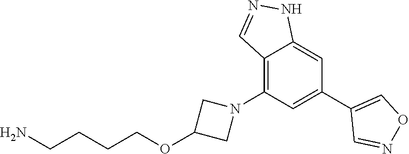 (Intermediate 2.115) | ¹H NMR (400 MHz, MeOH-d₄) δ 9.08 (s, 1H), 8.86 (s, 1H), 8.52 (s, 1H), 7.97 (d, J = 0.4 Hz, 1H), 7.25 (d, J = 8.8 Hz, 1H), 7.09 (s, 1H), 6.90 (d, J = 2.0 Hz, 1H), 6.75 (dd, J = 2.4, 9.2 Hz, 1H), 6.51 (s, 1H), 6.15 (d, J = 1.2 Hz, 1H), 4.62 (t, J = 7.2 Hz, 1H), 4.50-4.47 (m, 1H), 4.39 (t, J = 6.8 Hz, 2H), 4.29 (s, 2H), 3.96 (dd, J = 8.4, 4.0 Hz, 2H), 3.54 (t, J = 6.0 |
| | | | Hz, 2H), 3.10-3.06 (m, 2H), |
| | | | 2.46-2.42 (m, 2H), 2.13- |
| | | | 2.08 (m, 2H), 1.85-1.80 (m, |
| | | | 3H), 1.75-1.71 (m, 3H) ppm |
| | | | LCMS (AM3): rt = 0.874 min, |
| | | | (527.3 [M + H]⁺), 100% purity |
| | | | Purification Method PM78 |
| Example 124 | 4-((1-(6-(4H- 1,2,4-triazol-4-yl)-1H- indazol-4-yl)azetidin-3- yl)oxy)-N-((5- cyclobutoxy-1H- indol-2-yl)methyl) butan-1-amine | 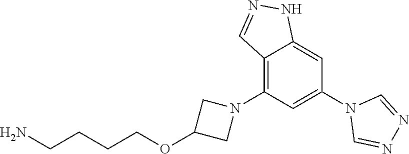 (Intermediate 2.221) | ¹H NMR (400 MHz, MeOH-d₄) δ 8.99 (s, 2H), 8.07 (d, J = 0.8 Hz, 1H), 7.16 (d, J = 8.8 Hz, 1H), 7.00 (s, 1H), 6.85 (d, J = 2.0 Hz, 1H), 6.64 (dd, J = 8.8 Hz, 2.4 Hz, 1H), 6.25 (s, 1H), 6.07 (d, J = 1.6 Hz, 1H), 4.65- 4.55 (m, 1H), 4.48-4.38 (m, 3H), 4.01 (dd, J = 8.4 Hz, 3.6 Hz, 2H), 3.88 (s, 2H), 3.51- 3.45 (m, 2H), 2.65 (t, J = 6.8 Hz, 2H), 2.45-2.37 (m, 2H), |
| | | | 2.15-2.04 (m, 2H), 1.85-1.75 |
| | | | (m, 1H), 1.74-1.66 (m, 1H), |
| | | | 1.66-1.61 (m, 4H) ppm |
| | | | LCMS (AM3): rt = 0.821 min, |
| | | | (527.3 [M + H]⁺), 100% purity |
| | | | Purification method 1) |
| | | | PM283 then 2) PM280 |

### Example 56

### 4-(2-((6-(pyridazin-4-yl)-1H-indazol-4-yl)oxy)ethoxy)-N-((5-(trifluoromethyl)-1H-indol-2-yl)methyl)butan-1-amine

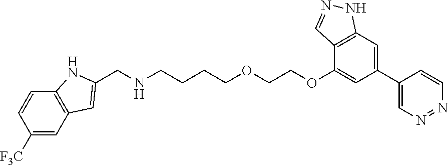 [SMILES: FC(F)(F)c1ccc2[nH]c(CNCCCCOCCOc3cc(-c4ccnnc4)cc4[nH]ncc34)cc2c1]

To a solution of compound 1.913 (255 mg, 0.54 mmol, HCl salt) in MeOH (2 mL) at 25° C. was added DIPEA (139.96 mg, 1.08 mmol) and 5-(trifluoromethyl)-1H-indole-2-carbaldehyde(Intermediate (CAS 1367793-79-0, 126.96 mg, 0.54 mmol). The reaction mixture was stirred for 11h. NaBH(OAc)₃ (1.15 g, 5.41 mmol) was added then the mixture stirred for 1 h. The mixture was then concentrated in vacuo to give a residue, which was purified (PM284) to afford EXAMPLE 56 (138.20 mg, 47.9% yield) as a yellow solid.

LCMS (AM3): rt=0.779 min, (525.0 [M+H]⁺), 98.5% purity.

¹H NMR: (400 MHz, MeOH-d₄) δ: 9.57 (m, 1H), 9.21-9.20 (m, 1H), 8.14 (m, 1H), 8.04-8.02 (m, 1H), 7.78-7.77 (m, 1H), 7.57-7.56 (m, 1H), 7.47-7.45 (m, 1H), 7.34-7.31 (m, 1H), 6.93 (s, 1H), 6.57 (s, 1H), 4.41-4.39 (m, 2H), 4.12-4.10 (m, 2H), 3.93-3.91 (m, 2H), 3.66-3.63 (m, 2H), 2.90 (s, 2H), 1.78-1.69 (m, 4H) ppm.

The following examples in Table 17 were made with non-critical changes or substitutions to the exemplified procedure for Example 56, that would be understood by one skilled in the art using 5-(trifluoromethyl)-1H-indole-2-carbaldehyde (CAS 1367793-79-0)

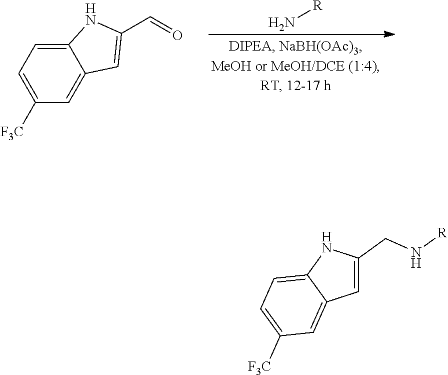

TABLE 17

| Example | Chemical IUPAC | | |
|---|---|---|---|
| No. | name | H₂NR | Analytical |
| Example 101 | 4-(2-((6- (pyridazin-4-yl)- 1H- benzo[d][1,2,3] triazol-4-yl) oxy)ethoxy)-N- ((5- (trifluoromethyl)- 1H-indol-2- yl)methyl)butan- 1-amine | 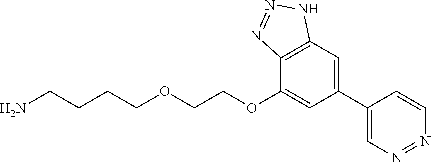 (Intermediate 2.367) | ¹H NMR (400 MHz, MeOH-d₄) δ 9.60 (d, J = 1.2 Hz, 1H), 9.16 (dd, J = 5.6, 1.2 Hz, 1H), 8.03 (dd, J = 5.2, 2.4 Hz, 1H), 7.92 (s, 1H), 7.80 (s, 1H), 7.51 (d, J = 8.4 Hz, 1H), 7.34 (d, J = 8.4 Hz, 1H), 7.00 (d, J = 1.2 Hz, 1H), 6.61 (s, 1H), 4.45- 4.38 (m, 2H), 4.31 (s, 2H), 3.98- 3.89 (m, 2H), 3.66 (t, J = 5.2 Hz, 2H), 3.11 (t, J = 7.2 Hz, 2H), 1.99- 1.88 (m, 2H), 1.83-1.72 (m, 2H) ppm |
| | | | LCMS (AM3): rt = 0.820 min, |
| | | | (526.3 [M + H]⁺), 99% purity |
| | | | Purification Method PM285 |
| Example 116 | 4-(2-((6-(4H- 1,2,4-triazol-4-yl)- 1H- benzo[d][1,2,3] triazol-4- yl)oxy)ethoxy)-N- ((5- (trifluoromethyl)- 1H-indol-2- yl)methyl)butan- 1-amine | 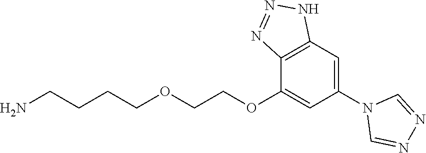 (Intermediate 2.347) | ¹H NMR (400 MHz, MeOH-d₄) δ 8.99 (s, 2H), 7.82 (s, 1H), 7.61 (d, J = 1.6 Hz, 1H), 7.54 (d, J = 8.4 Hz, 1H), 7.37 (dd, J = 8.4, 1.6 Hz, 1H), 6.82 (d, J = 1.6 Hz, 1H), 6.66 (s, 1H), 4.39-4.35 (m, 4H), 3.94-3.91 (m, 2H), 3.67 (t, J = 5.6 Hz, 2H), 3.17 (t, J = 7.2 Hz, 2H), 2.00-1.93 (m, 2H), 1.81-1.75 (m, 2H) ppm LCMS (AM3): rt = 0.794 min, (515.2 [M + H]⁺), 100.00% purity Purification Method PM286 |
| Example 117 | 4-(2-((6-(4H- 1,2,4-triazol-4-yl)- 1H-indazol-4- yl)oxy)ethoxy)-N- ((5- (trifluoromethyl)- 1H-indol-2- yl)methyl)butan- 1-amine | 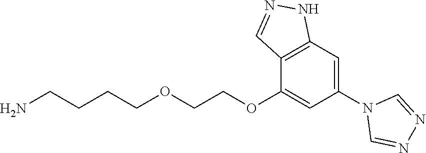 (Intermediate 1.374) | ¹H NMR (400 MHz, MeOH-d₄) δ 9.04 (s, 2H), 8.16 (s, 1H), 7.75 (s, 1H), 7.41 (d, J = 8.4 Hz, 1H), 7.36 (s, 1H), 7.30-7.27 (m, 1H), 6.82 (s, 1H), 6.44 (s, 1H), 4.39-4.36 (m, 2H), 3.91-3.88 (m, 4H), 3.61 (t, J = 5.6 Hz, 2H), 2.66 (t, J = 7.2 Hz, 2H), 1.67-1.62 (m, 4H) ppm LCMS (AM3): rt = 0.817 min, (514.0 [M + H]⁺), 99.8% purity Purification Method PM287 |
| Example 128 | 4-((1-(6-(4H-1,2,4- triazol-4-yl)-1H- indazol-4-yl)azetidin- 3-yl)oxy)-N-((5- (trifluoromethyl)- 1H-indol-2- yl)methyl)butan- 1-amine | 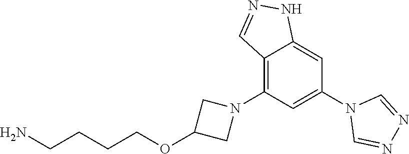 (Intermediate 2.221) | ¹H NMR (400 MHz, MeOH-d₄) δ 8.99 (s, 2H), 8.08 (s, 1H), 7.76 (s, 1H), 7.43 (d, J = 8.0 Hz, 1H), 7.29 (dd, J = 1.6 Hz, J = 8.8 Hz, 1H), 7.01 (s, 1H), 6.48 (s, 1H), 6.09 (d, J = 1.6 Hz, 1H), 4.50-4.43 (m, 3H), 4.06-4.02 (m, 2H), 3.94 (s, 2H), 3.53-3.50 (m, 2H), 2.70-2.66 (m, 2H), 1.68-1.64 (m, 4H) ppm LCMS (AM3): rt = 0.766 min, (525.2 [M + H]⁺), 100% purity Purification Method PM288 |
| Example 139 | 4-((1-(6- (isoxazol-4-yl)- 1H-indazol-4- yl)azetidin-3- yl)oxy)-N-((5- (trifluoromethyl)- 1H-indol-2- yl)methyl)butan- 1-amine | 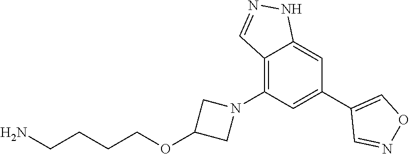 (Intermediate 2.115) | ¹H NMR (400 MHz, MeOH-d₄) δ 9.07 (s, 1H), 8.85 (s, 1H), 8.50 (s, 1H), 7.98 (s, 1H), 7.87 (s, 1H), 7.53 (d, J = 8.8 Hz, 1H), 7.40 (d, J = 8.8 Hz, 1H), 7.09 (s, 1H), 6.74 (s, 1H), 6.17 (s, 1H), 4.52-4.49 (m, 1H), 4.43 (t, J = 7.2 Hz, 2H), 4.36 (s, 2H), 3.99 (dd, J = 8.4, 4.0 Hz, 2H), 3.56 (t, J = 5.6 Hz, 2H), 3.10 (t, J = 7.6 Hz, 2H), 1.87-1.83 (m, 2H), 1.75-1.71 (m, 2H) ppm LCMS (AM3): rt = 0.835 min, |
| | | | (525.3 [M + H]⁺), 99.1% purity |
| | | | Purification Method PM289 |
| Example 140 | 4-((1-(6- (pyridazin-4-yl)- 1H-indazol-4- yl)azetidin-3- yl)oxy)-N-((5- (trifluoromethyl)- 1H-indol-2- yl)methyl)butan- 1-amine | 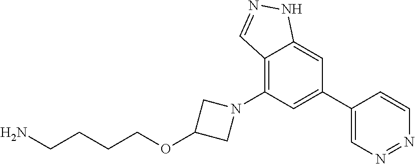 (Intermediate 2.113) | ¹H NMR (400 MHz, MeOH-d₄-d4) δ 9.55 (dd, J = 1.2, 2.4 Hz, 1H), 9.19 (dd, J = 1.2, 5.6 Hz, 1H), 8.07 (s, 1H), 8.01 (dd, J = 2.4, 5.6 Hz, 1H), 7.76 (s, 1H), 7.42 (d, J = 8.8 Hz, 1H), 7.33-7.25 (m, 2H), 6.48 (s, 1H), 6.28 (d, J = 1.2 Hz, 1H), 4.53- 4.40 (m, 3H), 4.03 (dd, J = 3.6, 8.4 Hz, 2H), 3.93 (s, 2H), 3.51 (t, J = 5.6 Hz, 2H), 2.67 (br t, J = 6.8 Hz, 2H), 1.70-1.61 (m, 4H) ppm LCMS (AM3): rt = 0.630 min, |
| | | | (536.1 [M + H]⁺), 96.88% purity |
| | | | Purification Method PM277 |
| Example 152 | 4-((1-(6-(4H- 1,2,4-triazol-4-yl)- 1H- benzo[d][1,2,3] triazol-4-yl)azetidin- 3-yl)oxy)-N-((5- (trifluoromethyl)- 1H-indol-2- yl)methyl)butan- 1-amine | 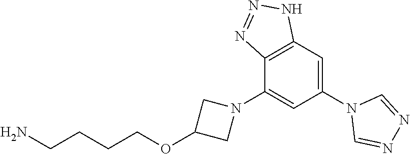 (Intermediate 2.304) | ¹H NMR (400 MHz, MeOH-d₄) δ 9.00 (s, 2H), 7.79 (s, 1H), 7.50- 7.41 (m, 1H), 7.35-7.26 (m, 1H), 7.17 (s, 1H), 6.55 (s, 1H), 6.23 (s, 1H), 4.60-4.50 (m, 3H), 4.20-4.10 (m, 2H), 4.04 (s, 2H), 3.52 (t, J = 6.0 Hz, 2H), 2.79 (t, J = 6.8 Hz, 2H), 1.78-1.60 (m, 4H) ppm LCMS (AM3): rt = 0.821 min, (526.2 [M + H]⁺), 100% purity. Purification Method PM293 |

### Example 57

### N-(2-(4-(((5-cyclobutoxy-1H-indol-2-yl)methyl)amino)butoxy)ethyl)-6-(pyridazin-4-yl)-1H-indazol-4-amine

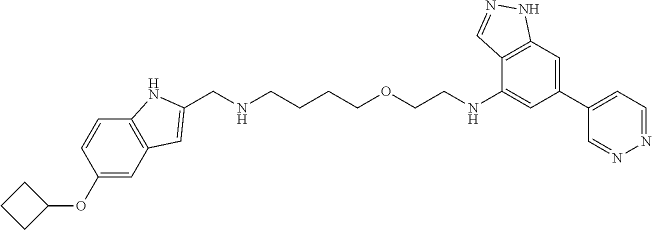 [SMILES: c1cc(-c2cc(NCCOCCCCNCc3cc4cc(OC5CCC5)ccc4[nH]3)c3cn[nH]c3c2)cnn1]

To a solution of compound 1.921 (300 mg, 826.77 μmol) and compound 1.420 (160.16 mg, 744.10 μmol) in MeOH (10 mL) was added DIPEA (320.56 mg, 2.48 mmol). The mixture was stirred at 20° C. for 12 h, then NaBH₃CN (519.56 mg, 8.27 mmol) was added and the reaction was stirred at 20° C. for 1 h. The reaction mixture was concentrated in vacuo to give a residue, which was purified (PM294) to afford EXAMPLE 57 (92.33 mg, 169.50 μmol, 20.5% yield) as a yellow solid.

LCMS (AM3): rt=0.814 min, (526.2 [M+H]⁺), 96.4% purity.

¹H NMR: (400 MHz, MeOH-d₄) δ: 9.53 (dd, J=2.8, 1.2 Hz, 1H), 9.16 (dd, J=5.6, 1.2 Hz, 1H), 8.19 (d, J=0.8 Hz, 1H), 7.97 (dd, J=5.6, 2.4 Hz, 1H), 7.19-7.14 (m, 2H), 6.83 (d, J=2.4 Hz, 1H), 6.64 (dd, J=8.8, 2.4 Hz, 1H), 6.52 (d, J=1.2 Hz, 1H), 6.23 (s, 1H), 4.62-4.55 (m, 1H), 3.84 (s, 2H), 3.72 (t, J=5.6 Hz, 2H), 3.53 (q, J=5.6 Hz, 4H), 2.64 (t, J=7.2 Hz, 2H), 2.45-2.37 (m, 2H), 2.14-2.04 (m, 2H), 1.82-1.66 (m, 2H), 1.63-1.61 (m, 4H) ppm

### Example 59

### 4-((3-(3-(((2-chloro-[1,1′-biphenyl]-4-yl)methyl)amino)propanamido)propyl)amino)-1H-indazole-6-carboxylic acid

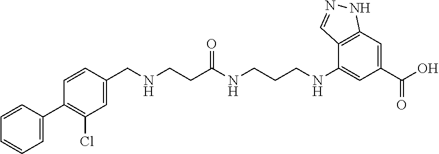 [SMILES: O=C(CCNCc1ccc(-c2ccccc2)c(Cl)c1)NCCCNc1cc(C(=O)O)cc2[nH]ncc12]

A mixture of compound 2.569 (100 mg, 192.30 μmol) in MeOH (2.0 mL) and NaOH (2.0 M in water, 3.50 mL) was stirred at 25° C. for 4 h. The solvent was removed under reduced pressure to give a residue, which was purified (PM295) to obtain 70 mg of crude product, then the crude product was further purified (PM296) to afford EXAMPLE 59 (20 mg, 37.5 μmol, 13.9% yield) as a white solid.

LCMS (AM21): rt=1.825 min, (506.2 [M+H]⁺), 95.47% purity.

¹H NMR (400 MHz, DMSO) δ 8.21 (s, 1H), 8.03-8.02 (m, 1H), 7.50 (s, 1H), 7.44-7.30 (m, 8H), 6.60 (s, 1H), 6.34 (s, 1H), 3.73 (s, 2H), 3.20-3.16 (m, 4H), 2.74-2.70 (m, 2H), 2.30-2.27 (m, 2H), 1.81-1.77 (m, 2H) ppm.

### Example 60

### 4-((4-((4-(((2-chloro-[1,1′-biphenyl]-4-yl)methyl)amino)butyl)amino)butyl)amino)-1H-indazole-6-carboxylic acid

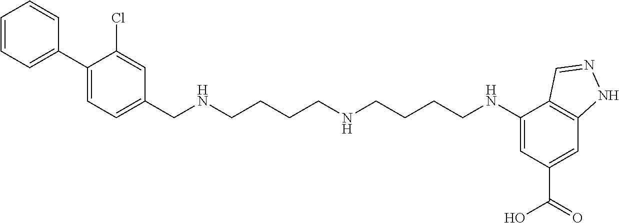 [SMILES: O=C(O)c1cc(NCCCCNCCCCNCc2ccc(-c3ccccc3)c(Cl)c2)c2cn[nH]c2c1]

To a solution of compound 2.574 (16.0 mg, 30.0 μmol) in AcOH (1.50 mL) was added HCl (1.50 mL) and the reaction was stirred at 90° C. for 2 h. The reaction mixture was concentrated in vacuo to give a residue, which was purified (PM297) to afford EXAMPLE 60 (10 mg, 18.27 μmol, 60.98% yield) as a grey solid.

LCMS (AM3): rt=0.742 min, (520.3 [M+H]⁺), 100% purity.

¹H NMR (400 MHz, D₂O) δ 8.10 (s, 1H), 7.68 (s, 1H), 7.50 (s, 1H), 7.40-7.33 (m, 8H), 4.13 (s, 2H), 3.30-3.27 (t, J=6.4 Hz, 2H), 2.99-2.90 (m, 6H), 1.68-1.62 (m, 8H) ppm.

### Example 61

### N-(3-((1H-indazol-4-yl)amino)propyl)-3-(((2-chloro-[1,1′-biphenyl]-4-yl)methyl)amino)propenamide

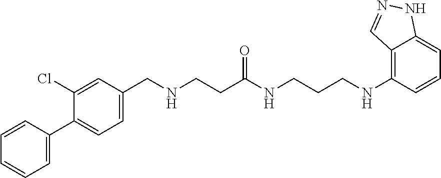 [SMILES: O=C(CCNCc1ccc(-c2ccccc2)c(Cl)c1)NCCCNc1cccc2[nH]ncc12]

A mixture of compound 2.357 (50 mg, 88.95 μmol) in HCl (2 M, 5 mL) was stirred at 25° C. for 16 h. The mixture was concentrated in vacuo to give a residue, which was purified (PM298) to afford EXAMPLE 61 (21.9 mg, 43.94 μmol, 49.39% yield, HCl salt) as a white solid.

LCMS (AM3): rt=0.806 min, (462.2 [M+H]⁺), 100.00% purity.

¹H NMR (400 MHz, MeOH-d₄) δ 8.49 (s, 1H), 7.71 (d, J=1.6 Hz, 1H), 7.58-7.33 (m, 10H), 4.30 (s, 2H), 3.56 (t, J=7.2 Hz, 2H), 3.40-3.34 (m, 4H), 2.74 (t, J=6.8 Hz, 2H), 2.05-1.98 (m, 2H) ppm

### Example 64

### N-(2-(2-(2-((3-chloro-4-(trifluoromethoxy)benzyl)amino)ethyl)oxazol-5-yl)ethyl)-6-(4H-1,2,4-triazol-4-yl)-1H-indazol-4-amine

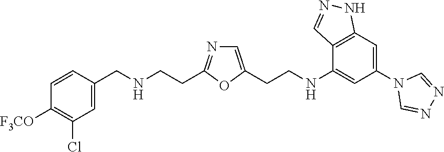 [SMILES: FC(F)(F)Oc1ccc(CNCCc2ncc(CCNc3cc(-n4cnnc4)cc4[nH]ncc34)o2)cc1Cl]

To a mixture of compound 2.069 (50 mg, 68.38 μmol) in DCM (2 mL) was added TFA (3.08 g, 27.01 mmol). The mixture was stirred at 20° C. for 1 h. The mixture was concentrated in vacuo to give a residue, which was purified (PM77) to afford EXAMPLE 64 (15.88 mg, 23.31 μmol, 34.08% yield, TFA salt) as a yellow oil.

LCMS (AM3): rt=0.792 min, (547.1 [M+H]⁺), 96.9% purity.

¹H NMR (400 MHz, MeOH-d₄) δ 9.00 (s, 2H), 8.15 (m, 1H), 7.77 (s, 1H), 7.55 (s, 2H), 6.95 (d, J=1.2 Hz, 1H), 6.90 (s, 1H), 6.28 (s, 1H), 4.29 (s, 2H), 3.65 (t, J=6.8 Hz, 2H), 3.43 (t, J=7.2 Hz, 2H), 3.30-3.12 (m, 4H) ppm

### Example 65

### N-(6-(4H-1,2,4-triazol-4-yl)-1H-indazol-4-yl)-2-(5-(2-((3-chloro-4-_(trifluoromethoxy)benzyl)amino)ethyl)-4H-1,2,4-triazol-3-yl)acetamide

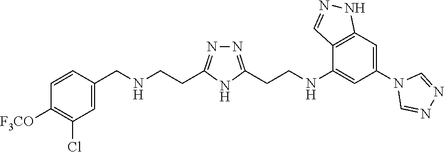 [SMILES: FC(F)(F)Oc1ccc(CNCCc2nnc(CCNc3cc(-n4cnnc4)cc4[nH]ncc34)[nH]2)cc1Cl]

To a mixture of compound 1.299 (50 mg, 67.10 μmol) in DCM (1 mL) was added TFA (1.54 g, 13.51 mmol, 1.00 mL). The mixture was stirred at 20° C. for 1 h. The solvent was removed to give a residue, which was purified (PM77) to afford EXAMPLE 65 (16.44 mg, 24.11 μmol, 35.94% yield, TFA salt) as a green solid.

LCMS (AM3): rt=0.763 min. (561.1[M+H]+), 99.27% purity

¹H NMR (400 MHz, MeOH-d₄) δ 9.05 (s, 2H), 8.40 (d, J=1.2 Hz, 1H), 8.02 (d, J=1.6 Hz, 1H), 7.78 (d, J=2.0 Hz, 1H), 7.63-7.59 (m, 1H), 7.58-7.55 (m, 1H), 7.53-7.48 (m, 1H), 4.33 (s, 2H), 4.21-4.10 (m, 2H), 3.51 (t, J=6.8 Hz, 2H), 3.20 (t, J=6.8 Hz, 2H) ppm

### Example 66

### N-(6-(4H-1,2,4-triazol-4-yl)-1H-indazol-4-yl)-3-((3-((3-chloro-4-(trifluoromethoxy)benzyl)amino)propyl)amino)propanamide

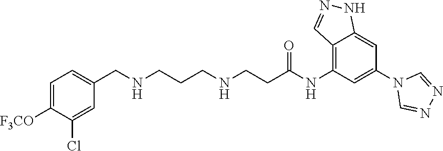 [SMILES: O=C(CCNCCCNCc1ccc(OC(F)(F)F)c(Cl)c1)Nc1cc(-n2cnnc2)cc2[nH]ncc12]

A mixture of compound 2.294 (30 mg, 39.94 μmol, TFA salt) and TFA (67.53 mmol, 5 mL) in DCM (5 mL) was stirred at 25° C. for 2 h. The reaction mixture was concentrated in vacuo to give a residue, which was purified (PM219) to afford EXAMPLE 66 (17.29 mg, 56.59% yield, TFA salt) as a white solid.

LCMS (AM3): rt=0.712 min, (537.1 [M+H]⁺), 100% purity.

¹H NMR (400 MHz, MeOH-d₄) δ 9.01 (s, 2H), 8.39 (s, 1H), 8.07 (d, J=1.6 Hz, 1H), 7.78 (s, 1H), 7.59 (s, 1H), 7.55 (s, 2H), 4.28 (s, 2H), 3.45 (t, J=5.6 Hz, 2H), 3.27-3.20 (m, 4H), 3.08 (t, J=6.8 Hz, 2H), 2.26-2.14 (m, 2H) ppm

### Example 67

### N1-(3-((6-(4H-1,2,4-triazol-4-yl)-1H-indazol-4-yl)oxy)propyl)-N3-(3-chloro-4-(trifluoromethoxy)benzyl)propane-1,3-diamine

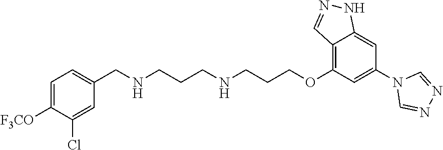 [SMILES: FC(F)(F)Oc1ccc(CNCCCNCCCOc2cc(-n3cnnc3)cc3[nH]ncc23)cc1Cl]

To a mixture of compound 2.363 (40 mg, 64.10 μmol) in DCM (2 mL) was added TFA (3.08 g, 27.01 mmol). The mixture was stirred at 25° C. for 0.5 h. The mixture was concentrated in vacuo to give a residue, which was purified (PM299) to afford EXAMPLE 67 (27.33 mg, 36.34 μmol, 56.70% yield, TFA salt) as a white solid.

LCMS (AM3): rt=0.746 min, (524.1 [M+H]⁺), 100% purity.

¹H NMR (400 MHz, MeOH-d₄) δ 9.05 (s, 2H), 8.16 (d, J=1.2 Hz, 1H), 7.78 (s, 1H), 7.60-7.51 (m, 2H), 7.42 (t, J=1.6 Hz, 1H), 6.84 (d, J=1.6 Hz, 1H), 4.39 (t, J=6.0 Hz, 2H), 4.28 (s, 2H), 3.38-3.34 (m, 2H), 3.26-3.19 (m, 4H), 2.38-2.29 (m, 2H), 2.24-2.14 (s, 2H) ppm

### Example 68

### N-(2-(4-(((6-chloro-1-methyl-1H-benzo[d]imidazol-2-yl)methyl)amino)butoxy)ethyl)-6-(4H-1,2,4-triazol-4-yl)-1H-indazol-4-amine

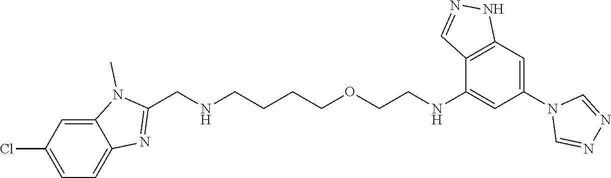 [SMILES: Cn1c(CNCCCCOCCNc2cc(-n3cnnc3)cc3[nH]ncc23)nc2ccc(Cl)cc21]

To a solution of compound 1.306 (100 mg, 284.23 μmol, HCl salt) in DMF (15 mL) was added K₂CO₃ (117.84 mg, 852.68 μmol) and 6-chloro-2-(chloromethyl)-1-methyl-1H-1,3-benzimidazole (CAS 109047-39-4, 30.57 mg, 142.11 μmol). The reaction mixture was stirred at 50° C. for 3 h. To the reaction mixture was added H₂O (15 mL) and then extracted with EA (20 mL×2). The combined organic phases were washed (brine, 30 mL), dried (Na₂SO₄), filtered and concentrated in vacuo to give a residue, which was purified (PM229) to afford EXAMPLE 68 (21.32 mg, 35.06 μmol, 12.34% yield, TFA salt) as a purple gum.

LCMS (AM3): rt=0.737 min, (494.4 [M+H]⁺), 100.00% purity

¹H NMR (400 MHz, MeOH-d₄) δ 9.16 (s, 2H), 8.21 (s, 1H), 7.55 (d, J=8.8 Hz, 1H), 7.49 (d, J=2.0 Hz, 1H), 7.18-7.15 (m, 1H), 6.94 (s, 1H), 6.19 (s, 1H), 4.50 (s, 2H), 3.81 (t, J=5.2 Hz, 2H), 3.67 (t, J=5.6 Hz, 2H), 3.61 (s, 3H), 3.53 (t, J=5.6 Hz, 2H), 3.33-3.30 (m, 2H), 1.97-1.91 (m, 2H), 1.86-1.80 (m, 2H) ppm

### Example 69

### 3-(3-((6-(4H-1,2,4-triazol-4-yl)-1H-indazol-4-yl)oxy)propoxy)-N-(3-chloro-4-(trifluoromethoxy)benzyl)propan-1-amine

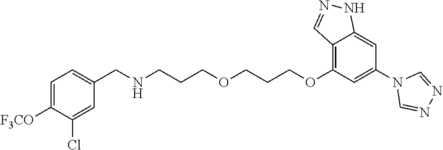 [SMILES: FC(F)(F)Oc1ccc(CNCCCOCCCOc2cc(-n3cnnc3)cc3[nH]ncc23)cc1Cl]

A mixture of compound 2.289 (94.27 mg, 267.18 μmol, HCl), 3-chloro-4-(trifluoromethoxy)benzaldehyde (CAS 83279-39-4, 60 mg, 267.18 μmol) and DIPEA (69.06 mg, 534.37 μmol) in MeOH (1 mL) was stirred at 25° C. for 1 h, then NaBH(OAc)₃ (283.14 mg, 1.34 mmol) was added. The mixture was stirred at 25° C. for 11 h. The mixture was filtered and the filtrate was concentrated in vacuo to give a residue, which was purified (PM214) to afford EXAMPLE 69 (68.53 mg, 107.25 μmol, 40.14% yield, TFA salt) as a brown gum.

LCMS (AM3): rt=0.716 min, (525.2 [M+H]⁺), 100.00% purity

¹H NMR (400 MHz, MeOH-d₄) δ 9.08 (s, 2H), 8.13 (d, J=0.8 Hz, 1H), 7.73 (d, J=2.0 Hz, 1H), 7.50 (t, J=2.0 Hz, 2H), 7.38 (s, 1H), 6.81 (s, 1H), 4.34 (t, J=6.0 Hz, 2H), 4.21 (s, 2H), 3.73 (t, J=6.0 Hz, 2H), 3.62 (t, J=5.6 Hz, 2H), 3.19 (t, J=7.2 Hz, 2H), 2.20-2.16 (m, 2H), 2.03-1.98 (m, 2H) ppm

The following examples in Table 18 were made with non-critical changes or substitutions to the exemplified procedure for Example 69, that would be understood by one skilled in the art using 3-chloro-4-(trifluoromethoxy)benzaldehyde

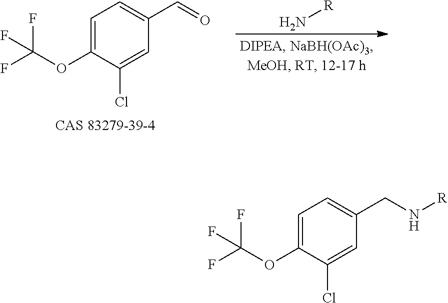

TABLE 18

| Example | Chemical IUPAC | | |
|---|---|---|---|
| No. | name | H₂NR | Analytical |
| Example 102 | N-(3-chloro-4- (trifluoromethoxy) benzyl)-4-(2-((6- (pyridazin-4-yl)- 1H-benzo[d][1,2,3] triazol-4-yl)oxy)ethoxy) butan-1-amine | 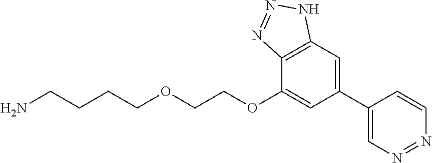 (Intermediate 2.367) | ¹H NMR (400 MHz, MeOH-d₄) δ 9.62 (dd, J = 2.8, 1.2 Hz, 1H), 9.20 (dd, J = 5.2, 1.2 Hz, 1H), 8.07 (dd, J = 5.6, 2.8 Hz, 1H), 7.91 (d, J = 0.8 Hz, 1H), 7.57 (d, J = 2.0 Hz, 1H), 7.39-7.27 (m, 2H), 7.08 (d, J = 1.2 Hz, 1H), 4.50- 4.41 (m, 2H), 4.08 (s, 2H), 3.99- 3.92 (m, 2H), 3.68 (t, J = 5.2 Hz, 2H), 3.08 (t, J = 7.2 Hz, 2H), 1.94- 1.85 (m, 2H), 1.83-1.73 (m, 2H) ppm |
| | | | LCMS (AM3): rt = 0.813 min, |
| | | | (537.0 [M + H]⁺), 97.13% purity |
| | | | Purification Method PM300 |
| Example 107 | N-(3-chloro-4- (trifluoromethoxy) benzyl)-4-((1-(6- (pyridazin-4-yl)- 1H-indazol-4- yl)azetidin-3- yl)oxy)butan-1- amine | 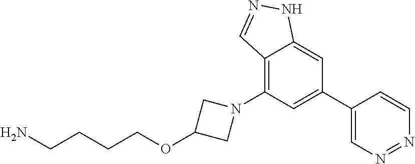 (Intermediate 2.113) | ¹H NMR (400 MHz, MeOH-d₄) δ 9.53 (dd, J = 1.2, 2.4 Hz, 1H) 9.17 (dd, J = 1.2, 5.6 Hz, 1H), 8.06 (d, J = 1.2 Hz, 1H), 7.99 (dd, J = 2.4, 5.6 Hz, 1H), 7.56 (s, 1H), 7.34 (s, 2H), 7.25 (s, 1H), 6.26 (d, J = 1.2 Hz, 1H), 4.54-4.42 (m, 3H), 4.03 (dd, J = 3.6, 8.4 Hz, 2H), 3.75 (s, 2H), 3.55-3.47 (m, 2H), 2.65-2.56 (m, 2H), 1.69- 1.60 (m, 4H) ppm LCMS (AM3): rt = 0.813 min, |
| | | | (547.2 [M + H]⁺), 100.00% purity |
| | | | Purification Method PM301 |
| Example 123 | 4-(2-((6-(4H-1,2,4- triazol-4-yl)-1H- benzo[d][1,2,3] triazol-4- yl)oxy)ethoxy)-N- (3-chloro-4- (trifluoromethoxy) benzyl)butan-1- amine | 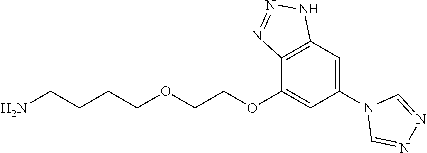 (Intermediate 2.347) | ¹H NMR (400 MHz, DMSO-d₆) δ 9.15 (s, 2H), 7.67 (s, 2H), 7.51- 7.45 (m, 2H), 7.00 (s, 1H), 4.50- 4.46 (m, 2H), 3.85-3.81 (m, 2H), 3.78 (s, 2H), 3.55-3.49 (m, 2H), 2.58-2.54 (m, 2H), 2.58-2.54 (m, 2H), 1.60-1.53 (m, 4H) ppm LCMS (AM3): rt = 0.801 min, (526.0 [M + H]⁺), 100.00% purity Purification Method PM229 |
| Example 132 | 4-((1-(6-(4H- 1,2,4-triazol-4-yl)- 1H-indazol-4- yl)azetidin-3- yl)oxy)-N-(3- chloro-4- (trifluoromethoxy) benzyl)butan-1- amine | 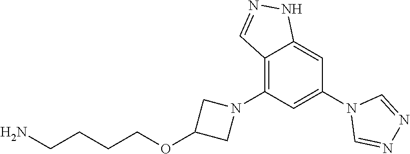 (Intermediate 2.221) | ¹H NMR (400 MHz, MeOH-d₄) δ 9.00 (s, 2H), 8.09 (d, J = 0.8 Hz, 1H), 7.57 (s, 1H), 7.38-7.33 (m, 2H), 7.04-6.98 (m, 1H), 6.11 (d, J = 1.6 Hz, 1H), 4.54-4.45 (m, 3H), 4.06 (dd, J = 8.4, 3.6 Hz, 2H), 3.76 (s, 2H), 3.55-3.49 (m, 2H), 2.66-2.57 (m, 2H), 1.69-1.62 (m, 4H) ppm LCMS (AM3): rt = 0.616 min, (536.1 [M + H]⁺), 95% purity Purification Method PM302 |
| Example 137 | N-(3-chloro-4- (trifluoromethoxy) benzyl)-4-((1-(6- (isoxazol-4-yl)- 1H-indazol-4- yl)azetidin-3- yl)oxy)butan-1- amine | 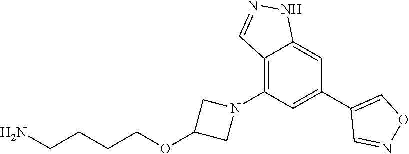 (Intermediate 2.115) | ¹H NMR (400 MHz, MeOH-d₄) δ 9.07 (s, 1H), 8.85 (s, 1H), 8.51 (s, 1H), 7.99 (s, 1H), 7.72 (s, 1H), 7.51-7.46 (m, 2H), 7.09 (s, 1H), 6.18 (s, 1H), 4.54-4.49 (m, 1H), 4.44 (t, J = 6.8 Hz, 2H), 4.15 (s, 2H), 4.00 (dd, J = 8.4, 4.0 Hz, 2H), 3.55 (t, J = 6.0 Hz, 2H), 3.04 (t, J = 7.2 Hz, 2H), 1.84-1.79 (m, 2H), 1.74-1.70 (m, 2H) ppm LCMS (AM3): rt = 0.821 min, (536.3 [M + H]⁺), 98.9% purity |
| | | | Purification Method PM289 |
| Example 153 | 4-((1-(6-(4H- 1,2,4-triazol-4-yl)- 1H-benzo[d][1,2,3] triazol-4-yl)azetidin- 3-yl)oxy)-N-(3- chloro-4- (trifluoromethoxy) benzyl)butan-1-amine | 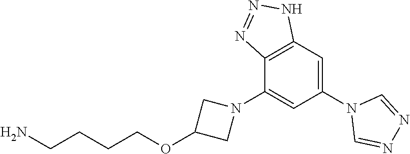 (Intermediate 2.304) | ¹H NMR (400 MHz, MeOH-d₄) δ 9.00 (s, 2H), 7.60 (s, 1H), 7.42- 7.33 (m, 2H), 7.16 (d, J = 1.6 Hz, 1H), 6.25 (d, J = 1.6 Hz, 1H), 4.60- 4.48 (m, 3H), 4.19-4.10 (m, 2H), 3.85 (s, 2H), 3.53 (t, J = 5.8 Hz, 2H), 2.72 (t, J = 7.2 Hz, 2H), 1.75-1.60 (m, 4H) ppm LCMS (AM3): rt = 0.824 min, (537.2 [M + H]⁺), 100% purity. Purification Method PM304 |

### Example 70

### N-(6-(4H-1,2,4-triazol-4-yl)-1H-indazol-4-yl)-3-(3-((3-chloro-4-(trifluoromethoxy)benzyl)amino)propoxy)propanamide

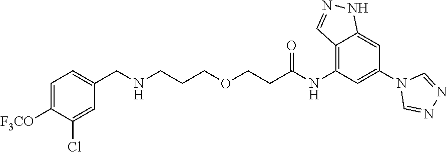 [SMILES: O=C(CCOCCCNCc1ccc(OC(F)(F)F)c(Cl)c1)Nc1cc(-n2cnnc2)cc2[nH]ncc12]

A mixture of compound 2.298 (180 mg, 492.05 μmol, HCl salt), 3-chloro-4-(trifluoromethoxy)benzaldehyde (CAS 83279-39-4, 23 mg, 547.73 μmol) and DIPEA (1.15 mmol, 200 μL) in MeOH (10 mL) was stirred at 25° C. for 16 h, then NaBH₃CN (137 mg, 2.18 mmol) was added and the reaction mixture was stirred at 25° C. for 2 h. The reaction mixture was concentrated in vacuo to give a residue, which was purified (PM305) to afford EXAMPLE 70 (76.72 mg, 23.92% yield, TFA salt) as a brown solid.

LCMS (AM3): rt=0.749 min, (538.1 [M+H]⁺), 100% purity.

¹H NMR (400 MHz, MeOH-d₄) δ 9.04 (s, 2H), 8.37 (s, 1H), 7.91 (s, 1H), 7.65 (d, J=2.0 Hz, 1H), 7.59 (d, J=1.2 Hz, 1H), 7.48-7.38 (m, 1H), 7.33-7.25 (m, 1H), 4.20 (s, 2H), 3.86 (t, J=5.6 Hz, 2H), 3.71 (t, J=5.6 Hz, 2H), 3.24 (t, J=5.6 Hz, 2H), 2.87 (t, J=5.6 Hz, 2H), 2.10-1.98 (m, 2H) ppm

### Example 71

### 3-(((2-chloro-[1,1′-biphenyl]-4-yl)methyl)amino)-N-(3-((6-(3-methyl-4H-1,2,4-triazol-4-yl)-1H-indazol-4-yl)amino)propyl)propanamide

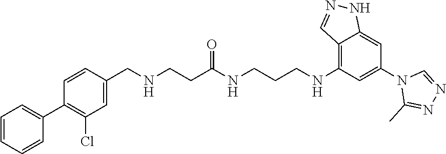 [SMILES: Cc1nncn1-c1cc(NCCCNC(=O)CCNCc2ccc(-c3ccccc3)c(Cl)c2)c2cn[nH]c2c1]

To a mixture of compound 2.072 (100 mg, 155.48 μmol) in DCM (3 mL) was added TFA (4.62 g, 40.52 mmol). The mixture was stirred at 30° C. for 0.5 h. The mixture was concentrated in vacuo to give a residue, which was purified (PM220) to afford EXAMPLE 71 (66.13 mg, 99.64 μmol, 64.08% yield, TFA salt) as a grey solid.

LCMS (AM3): rt=0.785 min, (543.4 [M+H]⁺), 99% purity.

¹H NMR (400 MHz, MeOH-d₄) δ 8.90 (s, 1H), 8.25 (d, J=1.2 Hz, 1H), 7.69 (d, J=1.6 Hz, 1H), 7.55-7.35 (m, 7H), 6.85 (s, 1H), 6.14 (d, J=1.6 Hz, 1H), 4.29 (s, 2H), 3.41-3.32 (m, 6H), 2.68 (t, J=6.4 Hz, 2H), 2.54 (s, 3H), 1.99-1.85 (m, 2H) ppm

The following examples in Table 19 were made with non-critical changes or substitutions to the exemplified procedure for Example 71 that would be understood by one skilled in the art

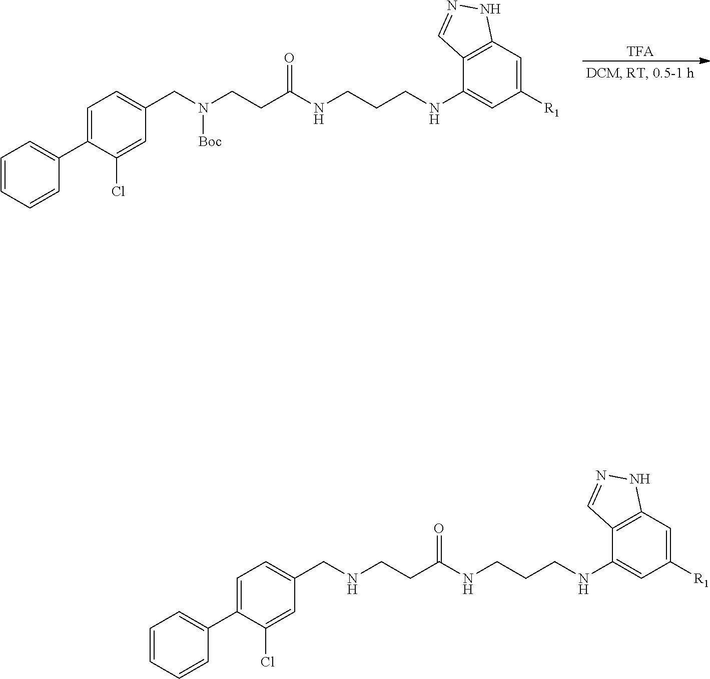

TABLE 19

| Example | Chemical | R₁ | |
|---|---|---|---|
| No. | IUPAC name | (Intermediate) | Analytical |
| Example 90 | 3-(((2-chloro-[1,1′-biphenyl]- 4-yl)methyl)amino)-N-(3- ((6-(tetrahydro-2H-pyran- 4-yl)-1H-indazol-4-yl) amino)propyl)propanamide | 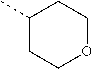 (Intermediate 2.277) | ¹H NMR (400 MHz, DMSO-d₆) δ 12.55 (br. s, 1H), 8.90 (br. s, 2H), 8.22 (s, 1H), 8.06 (s, 1 H), 7.75 (s, 1H), 7.55-7.42 (m, 7H), 6.50 (s, 1H), 5.95 (s, 1H), 4.23 (s, 2H), 3.96- 3.93 (m, 2H), 3.44-3.43 (m, 2H), 3.22-3.17 (m, 6H), 2.58-2.56 (m, 1H), 2.51-2.50 (m, 2H), 1.80-1.78 (m, 2H), 1.72-1.69 (m, 4H) ppm LCMS (AM3): rt = 0.847 min, (546.3 [M + H]⁺), 98.28% purity Purification Method PM223 |

### Example 72

### N1-(2-((6-(4H-1,2,4-triazol-4-yl)-1H-indazol-4-yl)amino)ethyl)-N4-(3-chloro-4-(trifluoromethoxy)benzyl)-N1-methylbutane-1,4-diamine

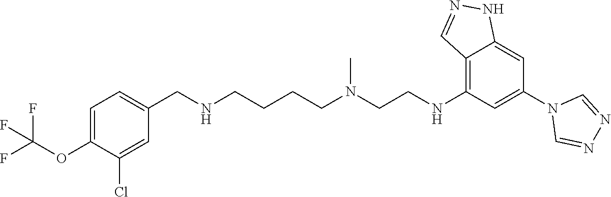 [SMILES: CN(CCCCNCc1ccc(OC(F)(F)F)c(Cl)c1)CCNc1cc(-n2cnnc2)cc2[nH]ncc12]

To a solution of compound 2.225 (90 mg, 141.27 μmol) in DCM (8 mL) was added TFA (12.32 g, 108.05 mmol). The reaction mixture was stirred at 20° C. for 0.5 h. The reaction mixture was concentrated in vacuo to give a residue, which was purified (PM306) and then further purified (PM307) to afford EXAMPLE 72 (31.77 mg, 59.16 μmol, 41.88% yield) as an off-white solid.

LCMS (AM3): rt=0.729 min, (537.4 [M+H]⁺), 100% purity

¹H NMR (400 MHz, MeOH-d₄) δ 9.08 (s, 2H), 8.19 (s, 1H), 7.76 (s, 1H), 7.54 (s, 2H), 7.10 (s, 1H), 6.46 (s, 1H), 4.23 (s, 2H), 3.81 (t, J=6.0 Hz, 2H), 3.65-3.45 (m, 2H), 3.28-3.20 (m, 2H), 3.11 (t, J=7.6 Hz, 2H), 2.99 (s, 3H), 1.90-1.78 (m, 4H) ppm

### Example 81

### 3-(((2-chloro-[1,1′-biphenyl]-4-yl)methyl)amino)-N-(3-((6-(oxetan-3-yl)-1H-indazol-4-yl)amino)propyl)propanamide

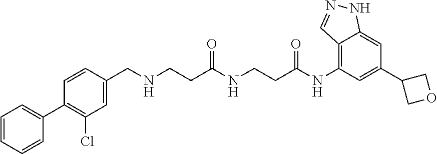 [SMILES: O=C(CCNCc1ccc(-c2ccccc2)c(Cl)c1)NCCC(=O)Nc1cc(C2COC2)cc2[nH]ncc12]

To a solution of compound 2.011 (60 mg, 0.085 mmol) in DCM (3 mL) was added TFA (2.31 g, 20.26 mmol). The mixture was stirred at 25° C. for 0.5 h. The mixture was neutralized to pH=7 with formic acid. The mixture was concentrated in vacuo to give a residue, which was purified (PM308) to afford EXAMPLE 82 (21.01 mg, 47.4% yield) as a yellow oil.

LCMS (AM3): rt=0.633 min, (518.1 [M+H]⁺), 100% purity.

¹H NMR: (400 MHz, CHCl₃-d) δ: 7.92 (s, 1H), 7.61-7.59 (m, 1H), 7.28-7.24 (m, 3H), 7.14-7.12 (m, 1H), 7.08-7.06 (m, 1H), 6.60 (s, 1H), 6.10 (s, 1H), 4.93-4.89 (m, 2H), 4.67-4.63 (m, 2H), 4.08-4.04 (m, 2H), 3.66 (s, 2H), 3.31-3.23 (m, 4H), 2.82-2.79 (m, 2H), 2.34-2.31 (m, 2H), 1.79-1.72 (m, 2H) ppm

### Example 83

### N-(3-((5-(4H-1,2,4-triazol-4-yl)-1H-indazol-7-yl)amino)propyl)-3-((3-chloro-4-(trifluoromethoxy)benzyl)amino)propanamide

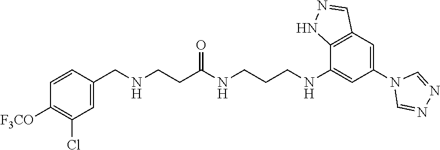 [SMILES: O=C(CCNCc1ccc(OC(F)(F)F)c(Cl)c1)NCCCNc1cc(-n2cnnc2)cc2cn[nH]c12]

A mixture of compound 2.081 (36 mg, 47.54 μmol) in TFA (4.62 g, 40.52 mmol) was stirred at 30° C. for 12 h. The mixture was concentrated in vacuo to give a residue, which was purified (PM309) to afford EXAMPLE 83 (13.98 mg, 20.40 μmol, 42.91% yield, TFA salt) as a yellow oil.

LCMS (AM3): rt=0.790 min, (537.1 [M+H]⁺), 95.4% purity.

¹H NMR (400 MHz, MeOH-d₄) δ 9.05 (s, 2H), 8.07 (s, 1H), 7.77 (d, J=1.2 Hz, 1H), 7.56-7.52 (M, 2H), 7.24 (d, J=1.6 Hz, 1H), 6.55 (d, J=1.6 Hz, 1H), 4.27 (s, 2H), 3.44-3.65 (m, 4H), 3.30-3.27 (m, 2H), 2.68 (t, J=6.4 Hz, 2H), 2.05-1.89 (m, 2H) ppm

### Example 84

### methyl4-((2-(4-(((5-(trifluoromethyl)-1H-indol-2-yl)methyl)amino)butoxy)ethyl)amino)-1H-indazole-6-carboxylate

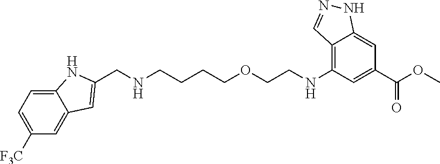 [SMILES: COC(=O)c1cc(NCCOCCCCNCc2cc3cc(C(F)(F)F)ccc3[nH]2)c2cn[nH]c2c1]

A mixture of 5-(trifluoromethyl)-1H-indole-2-carbaldehyde (CAS 1367793-79-0, 100 mg, 469.14 μmol), compound 2.105 (250.71 mg, 469.14 μmol) and DIPEA (121.26 mg, 938.28 μmol) in MeOH (10 mL) was stirred at 25° C. for 1 h, then NaBH₃CN (294.82 mg, 4.69 mmol) was added. The mixture was stirred at 25° C. for 11 h. The mixture concentrated in vacuo to give a residue, which was purified (PM310) to afford EXAMPLE 84 (60 mg, 117.97 μmol, 25.15% yield) as a brown solid.

LCMS (AM3): rt=0.870 min, (504.2 [M+H]⁺), 94.60% purity

¹H NMR (400 MHz, MeOH-d₄) δ 8.20 (br s, 1H), 7.88 (s, 1H), 7.50-7.57 (m, 2H), 7.42 (dd, J=8.8, 1.2 Hz, 1H), 6.65-6.94 (m, 2H), 4.34 (s, 2H), 3.90 (s, 3H), 3.73 (t, J=5.6 Hz, 2H), 3.59 (t, J=5.6 Hz, 2H), 3.49 (t, J=5.6 Hz, 2H), 3.10-3.16 (m, 2H), 1.81-1.89 (m, 2H), 1.70-1.75 (m, 2H) ppm

### Example 85

### 4-((2-(4-(((5-(trifluoromethyl)-1H-indol-2-yl)methyl)amino)butoxy)ethyl)amino)-1H-indazole-6-carboxylic acid

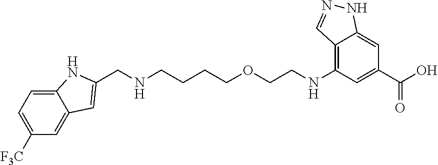 [SMILES: O=C(O)c1cc(NCCOCCCCNCc2cc3cc(C(F)(F)F)ccc3[nH]2)c2cn[nH]c2c1]

To a mixture of EXAMPLE 84 (50 mg, 99.30 μmol) in THF (1 mL) and MeOH (1 mL) and H₂O (1 mL) was added LiOH·H₂O (41.67 mg, 993.02 μmol). The mixture was stirred at 25° C. for 12 h. The mixture concentrated in vacuo to give a residue, which was purified (PM208) to afford EXAMPLE 85 (21.06 mg, 33.85 μmol, 34.09% yield) as a yellow solid.

LCMS (AM3): rt=0.789 min, (490.2 [M+H]⁺), 97.91% purity

¹H NMR (400 MHz, MeOH-d₄) δ 8.16 (d, J=0.8 Hz, 1H), 7.87 (s, 1H), 7.57-7.48 (m, 2H), 7.41 (dd, J=8.8, 1.6 Hz, 1H), 6.84 (d, J=0.8 Hz, 1H), 6.72 (s, 1H), 4.31 (s, 2H), 3.73 (t, J=5.6 Hz, 2H), 3.58 (t, J=5.6 Hz, 2H), 3.49 (t, J=5.6 Hz, 2H), 3.14-3.08 (m, 2H), 1.87-1.78 (m, 2H), 1.76-1.68 (m, 2H) ppm

### Example 86

### 6-(3-methyl-4H-1,2,4-triazol-4-yl)-N-(2-(4-(((5-(trifluoromethyl)-1H-indol-2-yl)methyl)amino)butoxy)ethyl)-1H-indazol-4-amine

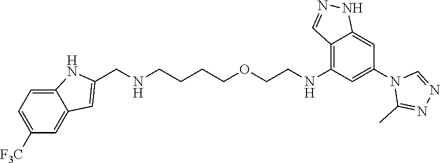 [SMILES: Cc1nncn1-c1cc(NCCOCCCCNCc2cc3cc(C(F)(F)F)ccc3[nH]2)c2cn[nH]c2c1]

To a mixture of compound 2.093 (50 mg, 136.66 μmol, HCl salt) and DIPEA (44.16 mg, 341.66 μmol) in MeOH (3 mL) was added 5-(trifluoromethyl)-1H-indole-2-carbaldehyde (CAS 1367793-79-0, 29.13 mg, 136.66 μmol). The mixture was stirred at 25° C. for 12 h. NaBH₃CN (85.88 mg, 1.37 mmol) was added and the mixture was stirred at 25° C. for 1 h. The mixture was concentrated in vacuo to give a residue, which was purified (PM309) to afford EXAMPLE 86 (14.48 mg, 19.00 μmol, 13.90% yield, TFA salt) as a brown solid.

LCMS (AM3): rt=0.811 min, (527.2 [M+H]⁺), 99% purity.

¹H NMR (400 MHz, DMSO-d₆) δ 8.78 (s, 1H), 8.23 (d, J=0.8 Hz, 1H), 7.88 (s, 1H), 7.55 (d, J=8.4 Hz, 1H), 7.43 (dd, J=8.4, 1.2 Hz, 1H), 6.84 (s, 1H), 6.75 (s, 1H), 6.15 (d, J=1.6 Hz, 1H), 4.36 (s, 2H), 3.71 (t, J=5.6 Hz, 2H), 3.56 (d, J=6.0 Hz, 1H), 3.46 (t, J=5.2 Hz, 2H), 3.18-3.05 (m, 2H), 2.49 (s, 3H), 1.88-1.76 (m, 2H), 1.76-1.64 (m, 2H) ppm

The following examples in Table 20 were made with non-critical changes or substitutions to the exemplified procedure for Example 86, that would be understood by one skilled in the art using 5-(trifluoromethyl)-1H-indole-2-carbaldehyde (CAS 1367793-79-0)

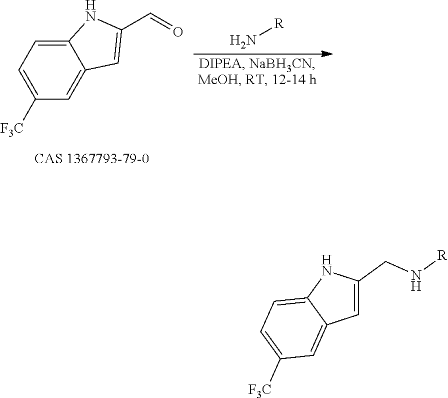

TABLE 20

| Example | Chemical | | |
|---|---|---|---|
| No. | IUPAC name | H₂NR | Analytical |
| Example 88 | methyl 3-(4-((2- (4-(((5- (trifluoromethyl)- 1H-indol-2-yl) methyl)amino) butoxy)ethyl) amino)-1H- indazol-6- yl)propanoate | 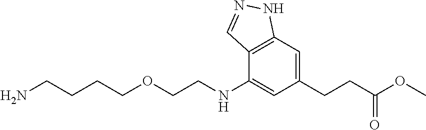 (Intermediate 2.352) | ¹H NMR (400 MHz, MeOH-d₄) δ 10.73 (br s, 1H), 8.10 (s, 1H), 7.63 (s, 1H), 7.39 (d, J = 8.4 Hz, 1H), 7.24 (dd, J = 0.8, 8.8 Hz, 1H), 6.20 (s, 1H), 5.93 (s, 1H), 4.39 (s, 2H), 3.75 (t, J = 5.6 Hz, 2H), 3.64-3.59 (m, 2H), 3.58 (s, 3H), 3.49 (t, J = 5.6 Hz, 2H), 3.06-2.93 (m, 4H), 2.49 (t, J = 7.6 Hz, 2H), 1.84-1.69 (m, 4H) ppm LCMS (AM3): rt = 0.863 min, (532.3 |
| | | | [M + H]⁺), 97.66% purity |
| | | | Purification Method PM311 |
| Example 92 | 6-(3-methoxy- 1H-pyrazol-4- yl)-N-(2-(4-(((5- (trifluoromethyl)-1H- indol-2-yl)methyl) amino)butoxy) ethyl)-1H-indazol- 4-amine | 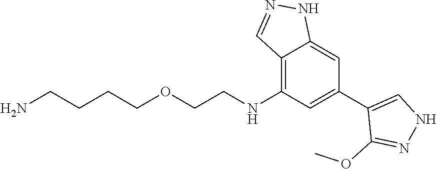 (Intermediate 2.086) | ¹H NMR (400 MHz, DMSO-d₆) δ 11.64 (s, 1H), 8.97 (s, 1H), 8.07 (s, 1H), 8.01-7.99 (m, 2H), 7.62 (d, J = 8.8 Hz, 1H), 7.43-7.41 (m, 1H), 7.04 (s, 1H), 6.75 (s, 1H), 6.35 (s, 1H), 4.32-4.31 (m, 2H), 3.92 (s, 3H), 3.65 (t, J = 5.6 Hz, 2H), 3.47 (t, J = 6.4 Hz, 2H), 3.41 (t, J = 6.0 Hz, 2H), 2.98 (d, J = 2.8 Hz, 2H), 1.72-1.66 (m, 2H), 1.61-1.57 (m, 2H) ppm LCMS (AM3): rt = 0.827 min, (542.4 [M + H]⁺), 100% purity |
| | | | Purification Method PM312 |
| Example 93 | 6-(pyridazin-4- yl)-N-(2-(4-(((5- (trifluoromethyl)- 1H-indol-2-yl) methyl)amino) butoxy)ethyl)-1H- indazol-4-amine | 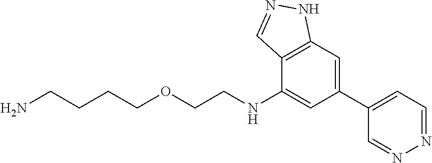 (Intermediate 1.921) | ¹H NMR (400 MHz, MeOH-d₄) δ 9.55 (d, J = 1.6 Hz, 1H), 9.20 (dd, J = 0.8, 5.6 Hz, 1H), 8.52 (br s, 1H), 8.21 (s, 1H), 8.01 (dd, J = 2.4, 5.6 Hz, 1H), 7.86 (s, 1H), 7.53 (d, J = 8.8 Hz, 1H), 7.40 (dd, J = 1.6, 8.5 Hz, 1H), 7.22 (s, 1H), 6.70 (s, 1H), 6.55 (d, J = 1.2 Hz, 1H), 4.29 (s, 2H), 3.78 (t, J = 5.6 Hz, 2H), 3.61-3.55 (m, 4H), 3.12- 3.04 (m, 2H), 1.86-1.79 (m, 2H), 1.75-1.70 (m, 2H) ppm LCMS (AM3): rt = 0.780 min, (524.3 |
| | | | [M + H]⁺), 95.811% purity |
| | | | Purification Method PM313 |
| Example 95 | 6-(pyrimidin-4- yl)-N-(2-(4-(((5- (trifluoromethyl)- 1H-indol-2- yl)methyl)amino) butoxy)ethyl)- 1H-indazol-4- amine | 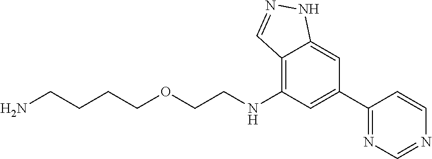 (Intermediate 2.117) | ¹H NMR (400 MHz, DMSO-d₆) δ 11.63 (s, 1H), 9.21 (s, 1H), 8.80 (d, J = 5.2 Hz, 1H), 8.25 (s, 1H), 8.21 (s, 1H), 8.08 (d, J = 4.4 Hz, 1H), 7.84 (s, 1H), 7.55 (s, 1H), 7.50 (d, J = 8.4 Hz, 1H), 7.33-7.30 (m, 1H), 6.88 (s, 1H), 6.48-6.43 (m, 2H), 3.94 (s, 2H), 3.65 (t, J = 6.0 Hz, 2H), 3.48- 3.44 (m, 4H), 2.61 (t, J = 6.4 Hz, 2H), 1.55 (s, 4H) ppm LCMS (AM3): rt = 0.835 min, (524.2 [M + H]⁺), 98.8% purity |
| | | | Purification method 1) PM78 then |
| | | | 2) PM79 |
| Example 97 | 6-(isoxazol-4- yl)-N-(2-(4-(((5- (trifluoromethyl)- 1H-indol-2- yl)methyl)amino) butoxy)ethyl)- 1H-indazol-4- amine | 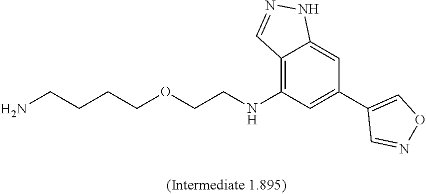 | ¹H NMR (400 MHz, DMSO-d₆) δ: 11.71 (s, 1H), 9.42 (s, 1H), 9.15 (s, 1H), 9.04 (s, 1H), 8.18 (s, 1H), 7.98 (s, 1H), 7.63-7.61 (m, 1H), 7.43- 7.41 (m, 1H), 6.98 (s, 1H), 6.75 (s, 1H), 6.36 (s, 1H), 4.33 (s, 2H), 3.67- 3.64 (m, 2H), 3.48-3.44 (m, 4H), 2.99 (s, 2H), 1.72-1.68 (m, 2H), 1.61-1.57 (m, 2H) ppm LCMS (AM3): rt = 0.759 min, (513.2 [M + H]⁺), 97.52% purity. Purification Method PM314 |
| Example 100 | 6-(pyridazin-4- yl)-N-(2-(4-(((5- (trifluoromethyl)- 1H-indol-2- yl)methyl)amino) butoxy)ethyl)-1H- benzo[d][1,2,3] triazol-4-amine | 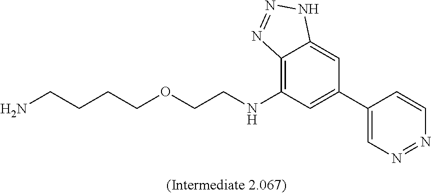 | ¹H NMR (400 MHz, MeOH-d₄) δ 9.58 (dd, J = 1.2, 2.4 Hz, 1H), 9.20 (dd, J = 1.2, 5.6 Hz, 1H), 8.03 (dd, J = 2.4, 5.6 Hz, 1H), 7.85-7.79 (m, 1H), 7.50-7.43 (m, 2H), 7.33 (dd, J = 1.2, 8.4 Hz, 1H), 6.73 (d, J = 1.2 Hz, 1H), 6.54 (s, 1H), 4.06 (s, 2H), 3.82-3.76 (m, 2H), 3.60 (td, J = 5.4, 13.6 Hz, 4H), 2.82 (t, J = 7.2 Hz, 2H), 1.83-1.66 (m, 4H) ppm LCMS (AM3): rt = 0.792 min, (525.1 [M + H]⁺), 97.7% purity |
| | | | Purification Method PM210 |
| Example 142 | 6-(4H-1,2,4- triazol-4-yl)-N- (2-(4-(((5- (trifluoromethyl)- 1H-indol-2- yl)methyl)amino) butoxy)ethyl)- 1H- benzo[d][1,2,3] triazol-4-amine | 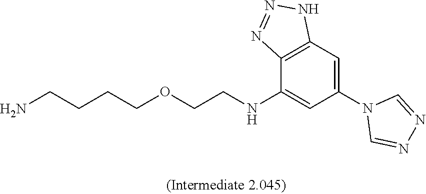 | ¹H NMR (400 MHz, MeOH-d₄) δ (s, 2H), 7.81 (s, 1H), 7.49 (d, J = 8.8 Hz, 1H), 7.34 (dd, J = 1.6, 8.4 Hz, 1H), 7.18 (d, J = 1.6 Hz, 1H), 6.57 (s, 1H), 6.49 (d, J = 1.6 Hz, 1H), 4.09 (s, 2H), 3.77 (t, J = 5.2 Hz, 2H), 3.61- 3.53 (m, 4H), 2.84 (t, J = 7.2 Hz, 2H), 1.80-1.67 (m, 4H) ppm LCMS (AM3): rt = 0.753 min, (514.2 [M + H]⁺), 100% purity Purification method 1) PM97 then 2) PM225 |
| Example 143 | 6-(isoxazol-4- yl)-N-(2-(4-(((5- (trifluoromethyl)- 1H-indol-2- yl)methyl)amino) butoxy)ethyl)-1H- benzo[d][1,2,3] triazol-4-amine | 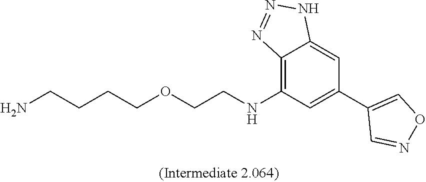 | ¹H NMR (400 MHz, MeOH-d₄) δ 9.14 (s, 1H), 8.91 (s, 1H), 8.53 (s, 1H), 7.85 (s, 1H), 7.53 (d, J = 8.4 Hz, 1H), 7.44-7.37 (m, 1H), 7.18 (d, J = 1.2 Hz, 1H), 6.70 (s, 1H), 6.66 (d, J = 1.2 Hz, 1H), 4.32 (s, 2H), 3.80 (t, J = 5.2 Hz, 2H), 3.65-3.58 (m, 4H), 3.12- 3.06 (m, 2H), 1.90-1.82 (m, 2H), 1.79-1.71 (m, 2H) ppm LCMS (AM3): rt = 0.810 min, (514.1 [M + H]⁺), 100% purity Purification method 1) PM88 then |
| | | | 2) PM290 |
| Example 144 | 4-(2-((6- (isoxazol-4-yl)- 1H-indazol-4- yl)oxy)ethoxy)- N-((5- (trifluoromethyl)- 1H-indol-2- yl)methyl)butan- 1-amine | 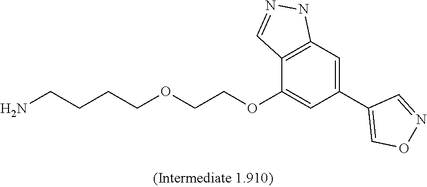 | ¹H NMR (400 MHz, MeOH-d₄) δ 9.14 (s, 1H), 8.90 (s, 1H), 8.50 (s, 1H), 8.06 (d, J = 0.8 Hz, 1H), 7.85 (s, 1H), 7.51 (d, J = 8.8 Hz, 1H), 7.41-7.34 (m, 2H), 6.80 (d, J = 0.8 Hz, 1H), 6.68 (s, 1H), 4.39-4.35 (m, 2H), 4.27 (s, 2H), 3.95-3.90 (m, 2H), 3.67 (t, J = 5.6 Hz, 2H), 3.08 (t, J = 7.6 Hz, 2H), 1.88-1.79 (m, 2H), 1.78- 1.72 (m, 2H) ppm LCMS (AM3): rt = 0.867 min, (514.2 [M + H]⁺), 100% purity |
| | | | Purification Method PM291 |
| Example 149 | 4-((1-(6- (pyridazin-4-yl)- 1H-benzo[d][1,2,3] triazol-4- yl)azetidin-3- yl)oxy)-N-((5- (trifluoromethyl)- 1H-indol-2- yl)methyl)butan- 1-amine | 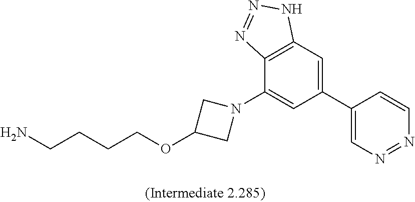 | ¹H NMR (400 MHz, MeOH-d₄) δ 9.58 (d, J = 2.4 Hz, 1H), 9.22-9.16 (m, 1H), 8.06-8.03 (m,, 1H), 7.79 (s, 1H), 7.56 (s, 1H), 7.44 (d, J = 8.8 Hz, 1H), 7.35-7.28 (m, 1H), 6.51 (s, 1H), 6.42 (s, 1H), 4.60-4.58 (m, 2H), 4.57-.4.53 (m, 1H), 4.15-4.07 (m, 2H), 3.97 (s, 2H), 3.57-3.52 (m, 2H), 2.76-2.69 (m, 2H), 1.71-1.66 (m, 4H) ppm LCMS (AM3): rt = 0.785 min, (537.2 [M + H]⁺), 97.95% purity Purification Method PM292 |

### Example 87

### 6-(2-methoxypyridin-4-yl)-N-(2-(4-(((5-(trifluoromethyl)-1H-indol-2-yl)methyl)amino)butoxy)ethyl)-1H-indazol-4-amine

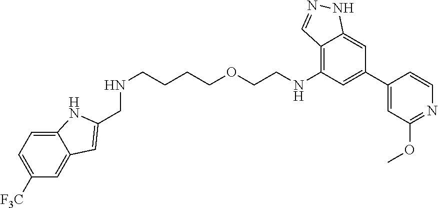 [SMILES: COc1cc(-c2cc(NCCOCCCCNCc3cc4cc(C(F)(F)F)ccc4[nH]3)c3cn[nH]c3c2)ccn1]

To a solution of compound 2.001 (100 mg, 281.35 μmol) in MeOH (1 mL) was added 5-(trifluoromethyl)-1H-indole-2-carbaldehyde (CAS 1367793-79-0, 53.97 mg, 0.25 mmol). After 11 h, NaBH₃CN (176.80 mg, 2.81 mmol) was added. The mixture was stirred at 25° C. for 1 h. The mixture was concentrated in vacuo to give a residue, which was purified (PM315) to afford EXAMPLE 87 (44.03 mg, 23.4% yield, TFA salt) as a yellow solid.

LCMS (AM3): rt=0.849 min, (553.3 [M+H]⁺), 99.7% purity.

¹H NMR: (400 MHz, MeOH-d₄) δ: 8.17-8.15 (m, 2H), 7.84 (s, 1H), 7.52-7.50 (m, 1H), 7.40-7.39 (m, 1H), 7.33-7.32 (m, 1H), 7.14 (s, 1H), 7.10 (s, 1H), 6.68 (s, 1H), 6.46 (s, 1H), 4.26 (s, 2H), 3.99 (s, 3H), 3.77-3.74 (m, 2H), 3.59-3.53 (m, 4H), 3.10-3.06 (m, 2H), 1.85-1.77 (m, 2H), 1.74-1.68 (m, 2H) ppm

The following examples in Table 21 were made with non-critical changes or substitutions to the exemplified procedure in Example 87, that would be understood by one skilled in the art using 5-(trifluoromethyl)-1H-indole-2-carbaldehyde (CAS 1367793-79-0)

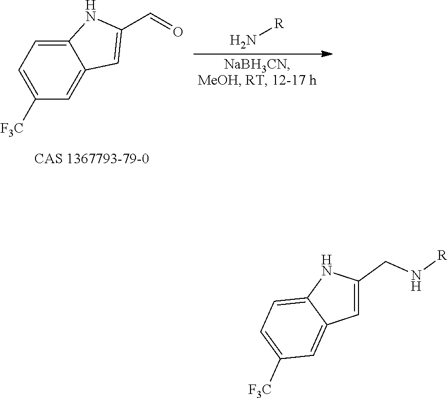

TABLE 21

| Example | Chemical IUPAC | | |
|---|---|---|---|
| No. | name | H₂NR | Analytical |
| Example 94 | N-(2-(4-(((5- (trifluoromethyl)-1H- indol-2-yl)methyl)amino) butoxy)ethyl)-1H- indazol-4-amine | 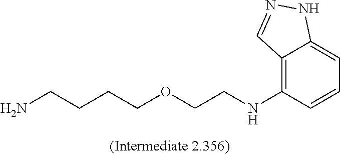 | ¹H NMR (400 MHz, MeOH-d₄) δ 8.58 (s, 1H), 8.12 (s, 1H), 7.86 (s, 1H), 7.52 (d, J = 8.8 Hz, 1H), 7.45- 7.35 (m, 1H), 7.18 (t, J = 8.0 Hz, 1H), 6.79 (d, J = 8.4 Hz, 1H), 6.63 (s, 1H), 6.20 (d, J = 7.6 Hz, 1H), 4.17 (s, 2H), 3.72 (t, J = 9.2 Hz, 2H), 3.56 (t, J = 8.8 Hz, 2H), 3.45 (t, J = 8.8 Hz, 2H), 3.00-2.91 (m, 2H), 1.84-1.62 (m, 4H) ppm LCMS (AM3): rt = 0.832 min, (446.3 [M + H]⁺), 100.00% purity Purification Method PM316 |

### Example 89

### 3-(4-((2-(4-(((5-(trifluoromethyl)-1H-indol-2-yl)methyl)amino)butoxy)ethyl)amino)-1H-indazol-6-yl)propanoic acid

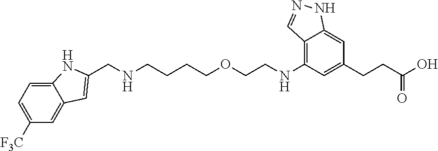 [SMILES: O=C(O)CCc1cc(NCCOCCCCNCc2cc3cc(C(F)(F)F)ccc3[nH]2)c2cn[nH]c2c1]

To a solution of EXAMPLE 88 (50 mg, 94.06 μmol) in THF (1 mL) and water (1 mL) was added LiOH (5.63 mg, 235.15 μmol). The reaction was stirred at 20° C. for 1 h. The reaction mixture was acidified to pH=7 by HCl (1N) and concentrated in vacuo to give a residue, which was purified (PM317) to afford EXAMPLE 89 (13.69 mg, 26.45 μmol, 28.12% yield, 100% purity) as a red oil.

LCMS (AM3): rt=0.838 min, (518.2 [M+H]⁺), 100.00% purity

¹H NMR (400 MHz, MeOH-d₄) δ 10.74 (s, 1H), 8.13 (s, 1H), 7.64 (s, 1H), 7.39 (d, J=8.4 Hz, 1H), 7.24 (dd, J=1.2, 8.4 Hz, 1H), 6.31 (s, 1H), 5.95 (s, 1H), 4.41 (s, 2H), 3.75 (t, J=5.6 Hz, 2H), 3.59 (t, J=5.6 Hz, 2H), 3.54-3.49 (m, 2H), 3.04-2.93 (m, 4H), 2.51 (t, J=7.8 Hz, 2H), 1.82-1.67 (m, 4H) ppm

### Example 96

### 4-((2-(4-(((5-(trifluoromethyl)-1H-indol-2-yl)methyl)amino)butoxy)ethyl)amino)-1H-indazole-6-carbonitrile

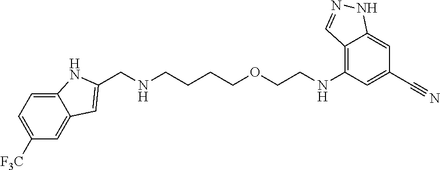 [SMILES: N#Cc1cc(NCCOCCCCNCc2cc3cc(C(F)(F)F)ccc3[nH]2)c2cn[nH]c2c1]

To a mixture of compound 2.108 (30 mg, 45.28 μmol) in MeOH (2 mL) was added K₂CO₃ (12.52 mg, 90.57 μmol). The mixture was stirred at 60° C. for 1 h. The mixture was concentrated in vacuo to give a residue, which was purified (PM318) to afford EXAMPLE 96 (6.05 mg, 11.48 μmol, 25.35% yield, FA salt) as a yellow solid.

LCMS (AM3): rt=0.854 min, (471.2 [M+H]⁺), 98.1% purity

¹H NMR (400 MHz, MeOH-d₄) δ 8.51 (br s, 1H), 8.21 (d, J=0.8 Hz, 1H), 7.86 (s, 1H), 7.53 (d, J=8.8 Hz, 1H), 7.40 (dd, J=8.8, 1.6 Hz, 1H), 7.14 (s, 1H), 6.70 (s, 1H), 6.35 (s, 1H), 4.28 (s, 2H), 3.70 (t, J=5.6 Hz, 2H), 3.56 (t, J=6.0 Hz, 2H), 3.49-3.44 (m, 2H), 3.09-3.03 (m, 2H), 1.83-1.77 (m, 2H), 1.71-1.68 (m, 2H) ppm

### Example 103

### 2-(3-(((4-((1-(6-(4H-1,2,4-triazol-4-yl)-1H-indazol-4-yl)azetidin-3-yl)oxy)butyl)amino)methyl)-5-fluorophenyl)acetonitrile

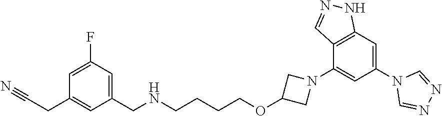 [SMILES: N#CCc1cc(F)cc(CNCCCCOC2CN(c3cc(-n4cnnc4)cc4[nH]ncc34)C2)c1]

To a solution of compound 2.221 (200 mg, 535.61 μmol) and 2-(3-Fluoro-5-formylphenyl)acetonitrile (Ref: WO2022185041, 87.38 mg, 535.61 μmol) in MeOH (2 mL) was added DIPEA (222.89 mg, 1.72 mmol). The mixture was stirred at 20° C. for 12 h, then NaBH(OAc)₃ (454.07 mg, 2.14 mmol) was added and the mixture was stirred at 20° C. for 1 h.

The reaction mixture was concentrated in vacuo to give a residue, which was purified (PM319) to afford EXAMPLE 103 (77.47 mg, 162.60 μmol, 30.36% yield) as a yellow gum.

LCMS (AM3): rt=0.682 min, (475.2 [M+H]⁺), 99.6% purity.

¹H NMR: (400 MHz, DMSO-d₆) δ 13.18 (s, 1H), 9.13 (s, 2H), 8.08 (s, 1H), 7.16 (s, 1H), 7.13 (d, J=9.6 Hz, 1H), 7.02-7.01 (m, 2H), 6.13 (d, J=1.6 Hz, 1H), 4.48-4.44 (m, 1H), 4.41 (t, J=6.8 Hz, 2H), 4.04 (s, 2H), 3.97 (dd, J=8.8, 3.6 Hz, 2H), 3.69 (s, 2H), 3.43-3.41 (m, 2H), 2.47-2.46 (m, 2H), 1.59-1.54 (m, 2H), 1.51-1.46 (m, 2H) ppm

The following examples in Table 22 were made with non-critical changes or substitutions to the exemplified procedure for Example 103, that would be understood by one skilled in the art, using Intermediate 2.221

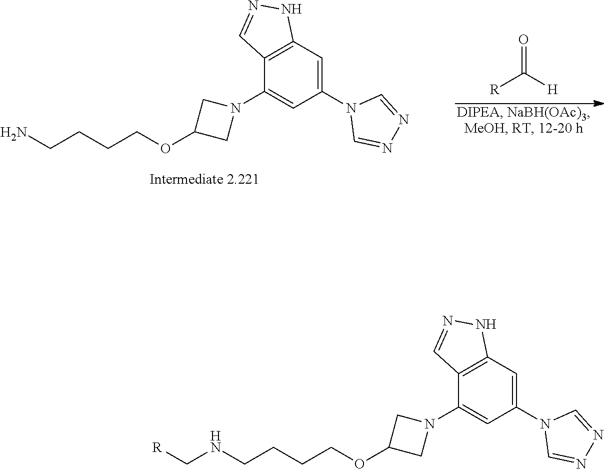

TABLE 22

| | | Aldehyde | |
|---|---|---|---|
| Example | Chemical IUPAC | Intermediate | |
| No. | name | No./CAS | Analytical |
| Example 104 | (3-(((4-((1-(6-(4H- 1,2,4-triazol-4-yl)- 1H-indazol-4- yl)azetidin-3- yl)oxy)butyl)amino) methyl)-5- (trifluoromethyl) phenyl)methanol | 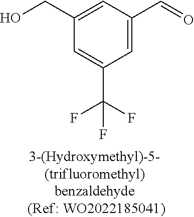 [SMILES: O=Cc1cc(CO)cc(C(F)(F)F)c1] | ¹H NMR (400 MHz, MeOH-d₄) δ 9.00 (s, 2H), 8.08 (d, J = 0.4 Hz, 1H), 7.58-7.55 (m, 3H), 7.01-6.99 (m 1H), 6.11 (d, J = 1.6 Hz, 1H), 4.66 (s, 2H), 4.54-4.50 (m, 1H), 4.49-4.45 (m, 2H), 4.07-4.02 (m, 2H), 3.83 (s, 2H), 3.54-3.49 (m, 2H), 2.63 (t, J = 6.8 Hz, 2H), 1.68-1.63 (m, 4H) LCMS (AM3): rt = 0.750 min, (516.2 [M + H]⁺), 99.2% purity Purification Method PM320 |
| Example 105 | 2-(3-(((4-((1-(6- (4H-1,2,4-triazol- 4-yl)-1H-indazol- 4-yl)azetidin-3- yl)oxy)butyl)amino) methyl)-5- (trifluoromethyl) phenyl)acetonitrile | 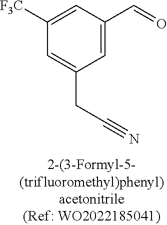 [SMILES: N#CCc1cc(C=O)cc(C(F)(F)F)c1] | ¹H NMR (400 MHz, MeOH-d₄) δ 8.99 (s, 2H), 8.08 (d, J = 0.8 Hz, 1H), 7.66 (s, 1H), 7.62 (s, 1H), 7.56 (s, 1H), 7.00 (s, 1H), 6.10 (d, J = 1.6 Hz, 1H), 4.52-4.50 (m, 1H), 4.47 (t, J = 7.2 Hz, 2H), 4.05 (dd, J = 8.4, 3.6 Hz, 2H), 3.85 (s, 2H), 3.52 (t, J = 5.6 Hz, 2H), 3.34 (s, 2H), 2.63 (t, J = 7.2 Hz, 2H), 1.68-1.65 (m, 4H) ppm LCMS (AM3): rt = 0.714 min, (525.3 [M + H]⁺), 98.9% purity Purification Method PM321 |
| Example 106 | 2-(3-(((4-((1-(6- (4H-12,4-triazol- 4-yl)-1H-indazol- 4-yl)azetidin-3- yl)oxy)butyl)amino) methyl)-5- methylphenyl) acetonitrile | 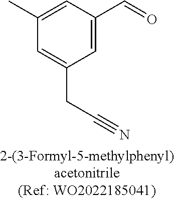 | ¹H NMR (400 MHz, MeOH-d₄) δ 8.99 (s, 2H), 8.07 (d, J = 0.8 Hz, 1H), 7.12 (s, 2H), 7.04 (s, 1H), 7.01-6.99 (m, 1H), 6.10 (d, J = 1.6 Hz, 1H), 4.52-4.49 (m, 1H), 4.48- 4.44 (m, 2H), 4.05-4.01 (m, 2H), 3.81 (s, 2H), 3.72 (s, 2H), 3.51 (t, J = 5.6 Hz, 2H), 2.62 (t, J = 6.8 Hz, 2H), 2.31 (s, 3H), 1.67- 1.62 (m, 4H) ppm LCMS (AM3): rt = 0.694 min, (471.2 [M + H]⁺), 100% purity Purification Method PM253 |
| Example 125 | (3-(((4-((1-(6-(4H- 1,2,4-triazol-4-yl)- 1H-indazol-4- yl)azetidin-3- yl)oxy)butyl)amino) methyl)-5- fluorophenyl) methanol | 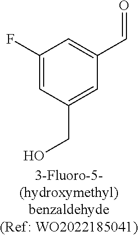 [SMILES: O=Cc1cc(F)cc(CO)c1] | ¹H NMR (400 MHz, MeOH-d₄) δ 9.00 (s, 2H), 8.08 (s, 1H), 7.12 (s, 1H), 7.01-6.93 (m, 3H), 6.11 (d, J = 1.6 Hz, 1H), 4.58 (s, 2H), 4.54-4.42 (m, 3H), 4.09-4.01 (m, 2H), 3.76 (s, 2H), 3.52 (t, J = 5.6 Hz, 2H), 2.62 (t, J = 7.2 Hz, 2H), 1.70-1.60 (m, 4H) ppm LCMS (AM3): rt = 0.729 min, (466.2 [M + H]⁺), 100% purity. Purification method 1) PM254 then 2) PM255 |
| Example 129 | 4-((6-(6-(4H- 1,2,4-triazol-4-yl)- 1H-indazol-4-yl) azetidine-3-yl)oxy)- N-(3-fluoro-4- (trifluoromethoxy) benzyl)butan-1-amine | 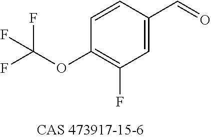 [SMILES: O=Cc1ccc(OC(F)(F)F)c(F)c1] | ¹H NMR (400 MHz, MeOH-d₄) δ 9.00 (s, 2H), 8.08 (d, J = 0.8 Hz, 1H), 7.35-7.31 (m, 2H), 7.32-7.23 (m, 1H), 7.01 (s, 1H), 6.11 (d, J = 1.6 Hz, 1H), 4.53-4.46 (m, 3H), 4.06 (dd, J = 3.6 Hz, J = 8.6 Hz, 2H), 3.77 (s, 2H), 3.54-3.50 (m, 2H), 2.63- 2.59 (m, 2H), 1.67-1.63 (m, 4H) ppm LCMS (AM3): rt = 0.751 min, (520.2 [M + H]⁺), 99.80% purity Purification Method PM280 |
| Example 130 | (3-(((4-((1-(6-(4H- 1,2,4-triazol-4-yl)- 1H-indazol-4- yl)azetidin-3- yl)oxy)butyl)amino) methyl)-5- chlorophenyl) methanol | 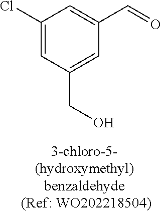 [SMILES: O=Cc1cc(Cl)cc(CO)c1] | ¹H NMR (400 MHz, MeOH-d₄) δ 9.00 (s, 2H), 8.08-8.07 (m, 1H), 7.27-7.22 (m, 3H), 7.01-6.99 (m, 1H), 6.11-6.09 (m, 1H), 4.56 (s, 2H), 4.51-4.44 (m, 3H), 4.06-4.02 (m, 2H), 3.74 (s, 2H), 3.51 (s, 2H), 2.63-2.59 (m, 2H), 1.65 (quin, J = 3.2 Hz, 4H) ppm LCMS (AM3): rt = 0.690 min, (482.1 [M + H]⁺), 99.39% purity Purification method 1) PM322 then 2) PM323 |
| Example 131 | 2-(3-(((4-((1-(6- (4H-1,2,4-triazol- 4-yl)-1H-indazol- 4-yl)azetidin-3- yl)oxy)butyl)amino) methyl)-5- chlorophenyl) acetonitrile | 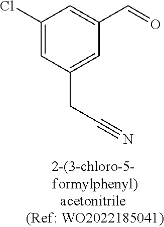 [SMILES: N#CCc1cc(Cl)cc(C=O)c1] | ¹H NMR (400 MHz, MeOH-d₄) δ 9.00 (s, 2H), 8.08 (d, J = 1.2 Hz, 1H), 7.36 (t, J = 1.6 Hz, 1H), 7.29-7.26 (m, 1H), 7.26- 7.23 (m, 1H), 7.03-6.98 (m, 1H), 6.11 (d, J = 1.6 Hz, 1H), 4.53-4.45 (m, 3H), 4.04 (dd, J = 8.4, 3.6 Hz, 2H), 3.76 (s, 2H), 3.54-3.49 (m, 2H), 3.34-3.32 (m, 2H), 2.64-2.58 (m, 2H), 1.68-1.61 (m, 4H) ppm LCMS (AM3): rt = 0.553 min, (491.1 [M + H]⁺), 100% purity. Purificaton Method PM324 |
| Example 147 | 2-(3-(((4-((1-(6- (4H-1,2,4-triazol- 4-yl)-1H-indazol- 4-yl)azetidin-3- yl)oxy)butyl)amino) methyl)-5- (trifluoromethoxy) phenyl)acetonitrile | 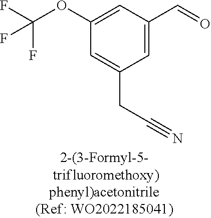 [SMILES: N#CCc1cc(C=O)cc(OC(F)(F)F)c1] | ¹H NMR (400 MHz, DMSO-d₆) δ 9.11 (s, 2H), 8.08 (s, 1H), 7.36 (s, 1H), 7.29 (s, 1H), 7.19 (s, 1H), 7.02 (s, 1H), 6.13 (s, 1H), 4.46-4.43 (m, 1H), 4.43-4.39 (m, 2H), 4.09 (s, 2H), 3.99-3.95 (m, 2H), 3.73 (s, 2H), 3.42 (t, J = 6.4 Hz, 2H), 2.49 (t, J = 6.4 Hz, 2H), 1.60-1.54 (m, 2H), 1.52-1.46 (m, 2H) ppm LCMS (AM3): rt = 0.767 min, (541.5 [M + H]⁺), 98.6% purity Purification Method PM321 |

### Example 108

### N-(3-fluoro-4-(trifluoromethoxy) benzyl)-4-((1-(6-(pyridazin-4-yl)-1H-indazol-4-yl) azetidin-3-yl) oxy) butan-1-amine

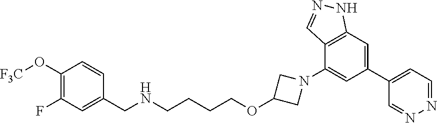 [SMILES: Fc1cc(CNCCCCOC2CN(c3cc(-c4ccnnc4)cc4[nH]ncc34)C2)ccc1OC(F)(F)F]

A mixture of compound 2.113 (200 mg, 591.01 μmol), 3-fluoro-4-(trifluoromethoxy)benzaldehyde (CAS 473917-15-6, 122.99 mg, 591.01 μmol) and DIPEA (229.14 mg, 1.77 mmol) in MeOH (3 mL) was stirred at 25° C. for 1 h, then sodium triacetoxyborhydride (375.77 mg, 1.77 mmol) was added. The mixture was stirred at 25° C. for 11 h. The mixture was concentrated in vacuo to give a residue, which was purified (PM325) to afford EXAMPLE 108 (20.82 mg, 38.46 μmol, 6.51% yield) as a yellow gum.

LCMS (AM3): rt=0.612 min, (531.1 [M+H]⁺), 98.8% purity

¹H NMR (400 MHz, MeOH-d₄) δ 9.53 (dd, J=2.4, 1.2 Hz, 1H), 9.17 (dd, J=5.6, 1.2 Hz, 1H), 8.06 (d, J=1.2 Hz, 1H), 7.99 (dd, J=5.6, 2.4 Hz, 1H), 7.38-7.29 (m, 2H), 7.28-7.17 (m, 2H), 6.26 (d, J=1.2 Hz, 1H), 4.56-4.43 (m, 3H), 4.03 (dd, J=8.4, 3.6 Hz, 2H), 3.78 (s, 2H), 3.55-3.48 (m, 2H), 2.62 (t, J=7.2 Hz, 2H), 1.70-1.60 (m, 4H) ppm

The following examples in Table 23 were made with non-critical changes or substitutions to the exemplified procedure for Example 108, that would be understood by one skilled in the art, using Intermediate 2.113

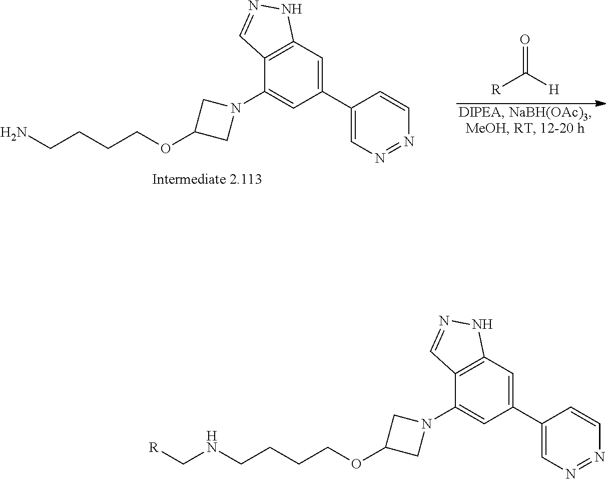

TABLE 23

| | | Aldehyde | |
|---|---|---|---|
| Example | Chemical | Intermediate | |
| No. | IUPAC name | No./CAS | Analytical |
| Example 110 | (3-fluoro-5- (((4-((1-(6- (pyridazin-4- yl)-1H-indazol- 4-yl)azetidin-3- yl)oxy)butyl)a- mino)methyl)phe- nyl)methanol | 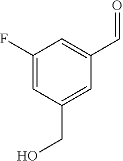 [SMILES: O=Cc1cc(F)cc(CO)c1] | ¹H NMR (400 MHz, MeOH-d₄) δ 9.55 (dd, J = 1.2, 2.4 Hz, 1H), 9.19 (dd, J = 1.2, 5.6 Hz, 1H), 8.07 (d, J = 0.8 Hz, 1H), 8.02 (dd, J = 2.4, 5.6 Hz, 1H), 7.28 (s, 1H), 7.12 (s, 1H), 6.99 (t, J = 8.8 Hz, 2H), 6.29 (d, J = 1.2 Hz, 1H), 4.58 (s, 2H), 4.51 (s, 1H), 4.50-4.44 (m, 2H), 4.03 (dd, J = 3.6, 8.4 Hz, 2H), 3.79 (s, 2H), 3.56-3.49 (m, 2H), 2.70-2.61 (m, 2H), 1.69-1.61 (m, 4H) ppm LCMS (AM3): rt = 0.730 min, (477.3 |
| | | 3-Fluoro-5- | [M + H]⁺), 99.25% purity |
| | | (hydroxymethyl) | Purification method 1) PM303 then 2) |
| | | benzaldehyde | PM326 |
| | | (Ref: | |
| | | WO2022185041) | |
| Example 111 | (3-chloro-5- (((4-((1-(6- (pyridazin-4- yl)-1H-indazol- 4-yl)azetidin-3- yl)oxy)butyl)a- mino)methyl)phe- nyl)methanol | 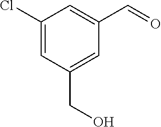 [SMILES: O=Cc1cc(Cl)cc(CO)c1] | ¹H NMR (400 MHz, MeOH-d₄) δ 9.53 (dd, J = 1.2, 2.4 Hz, 1H), 9.18 (dd, J = 1.2, 5.6 Hz, 1H), 8.55 (s, 1H), 8.06 (d, J = 0.8 Hz, 1H), 8.00 (dd, J = 2.4, 5.6 Hz, 1H), 7.33-7.27 (m, 3H), 6.26 (d, J = 1.2 Hz, 1H), 4.57 (s, 2H), 4.53-4.44 (m, 3H), 4.01 (dd, J = 3.6, 8.4 Hz, 2H), 3.94 (s, 2H), 3.53 (t, J = 5.6 Hz, 2H), 2.83 (t, J = 7.6 Hz, 2H), 1.77-1.66 |
| | | 3-Fluoro-5- | (m, 4H) ppm |
| | | (hydroxymethyl) | LCMS (AM3): rt = 0.754 min, (493.3 |
| | | benzaldehyde | [M + H]⁺), 99.32% purity |
| | | (Ref: | Purification method 1) PM327 then 2) |
| | | WO2022185041) | PM328 |
| Example 112 | (3-(((4-((1-(6- (pyridazin-4- yl)-1H-indazol- 4-yl)azetidin-3- yl)oxy)butyl)a- mino)methyl)- 5- (trifluoromethyl) phenyl)metha- | 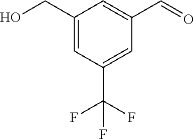 [SMILES: O=Cc1cc(CO)cc(C(F)(F)F)c1] | ¹H NMR (400 MHz, MeOH-d₄) δ 9.54 (dd, J = 1.2, 2.4 Hz, 1H), 9.18 (dd, J = 1.2, 5.6 Hz, 1H), 8.06 (d, J = 0.8 Hz, 1H), 8.01 (dd, J = 2.4, 5.6 Hz, 1H), 7.56 (d, J = 3.6 Hz, 3H), 7.27 (s, 1H), 6.27 (d, J = 1.2 Hz, 1H), 4.65 (s, 2H), 4.53-4.49 (m, 1H), 4.49-4.44 (m, 2H), 4.03 (dd, J = 4.0, 8.4 Hz, 2H), 3.83 (s, 2H), 3.55-3.48 (m, 2H), 2.63 (t, J = 6.8 Hz, 2H), 1.69-1.61 (m, 4H) ppm |
| | nol | 3- | LCMS (AM11): rt = 0.770 min, (527.3 |
| | | (Hydroxymethyl)- | [M + H]⁺), 100.00% purity |
| | | 5- | Purification method 1) PM329 then 2) |
| | | (trifluoromethyl) | PM330 |
| | | benzaldehyde, | |
| | | (Ref: | |
| | | WO2022185041) | |
| Example 113 | 2-(3-fluoro-5- (((4-((1-(6- (pyridazin-4- yl)-1H-indazol- 4-yl)azetidin-3- yl)oxy)butyl)a- mino)methyl)phe- nyl)acetonitrile | 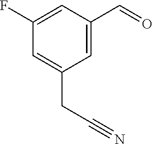 [SMILES: N#CCc1cc(F)cc(C=O)c1] | ¹H NMR (400 MHz, DMSO-d₆) δ 13.18 (br s, 1H), 9.65 (dd, J = 1.2, 2.4 Hz, 1H), 9.24 (dd, J = 1.2, 5.6 Hz, 1H), 8.09 (s, 1H), 8.01 (dd, J = 2.4, 5.6 Hz, 1H), 7.28 (s, 1H), 7.20- 7.09 (m, 2H), 7.02 (d, J = 9.2Hz, 1H), 6.30 (d, J = 1.2 Hz, 1H), 4.50-4.39 (m, 3H), 4.04 (s, 2H), 3.97 (dd, J = 3.6, 8.4 Hz, 2H), 3.70 (s, 2H), 3.43-3.40 (m, 2H), 2.49-2.47 (m, 2H), 1.61-1.46 (m, 4H) ppm |
| | | 2-(3-Fluoro-5- | LCMS (AM3): rt = 0.744 min, (486.2 |
| | | formylphenyl)ace- | [M + H]⁺), 99.16% purity |
| | | tonitrile (Ref: | Purification method 1) PM329 then 2) |
| | | WO2022185041) | PM319 |
| Example 114 | 2-(3-(((4-((1- (6-(pyridazin- 4-yl)-1H- indazol-4- yl)azetidin-3- yl)oxy)butyl)a- mino)methyl)- 5- (trifluoromethyl) | 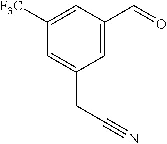 [SMILES: N#CCc1cc(C=O)cc(C(F)(F)F)c1] | ¹H NMR (400 MHz, DMSO-d₆) δ 13.18 (br s, 1H), 9.65 (dd, J = 1.2, 2.4 Hz, 1H), 9.24 (dd, J = 0.8, 5.6 Hz, 1H), 8.09 (s, 1H), 8.01 (dd, J = 2.4, 5.6 Hz, 1H), 7.65 (d, J = 12.4 Hz, 2H), 7.56 (s, 1H), 7.28 (s, 1H), 6.30 (s, 1H), 4.50-4.39 (m, 3H), 4.15 (s, 2H), 3.97 (dd, J = 3.6, 8.4 Hz, 2H), 3.78 (s, 2H), 3.42 (t, J = 6.4 Hz, 2H), 2.49-2.48 (m, 2H), 1.61- 1.47 (m, 4H) ppm |
| | phenyl)acetoni- | 2-(3-Formyl-5- | LCMS (AM3): rt = 0.776 min, (536.2 |
| | trile | (trifluoromethyl) | [M + H]⁺), 100.00% purity |
| | | phenyl)acetonitrile | Purification method 1) PM327 then 2) |
| | | (Ref: | PM331 |
| | | WO2022185041) | |
| Example 115 | 2-(3-methyl-5- (((4-((1-(6- (pyridazin-4- yl)-1H-indazol- 4-yl)azetidin-3- yl)oxy)butyl)a- mino)methyl)phe- nyl)acetonitrile | 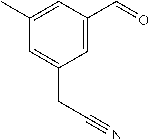 | ¹H NMR (400 MHz, DMSO-d₆) δ 9.65 (dd, J= 1.2, 2.4 Hz, 1H), 9.24 (dd, J = 1.2, 5.6 Hz, 1H), 8.09 (d, J = 0.8 Hz, 1H), 8.01 (dd, J = 2.8, 5.6 Hz, 1H), 7.28 (s, 1H), 7.08 ( d, J = 4.8 Hz, 2H), 6.97 (s, 1H), 6.30 (d, J = 1.2 Hz, 1H), 4.49-4.38 (m, 3H), 3.97 (dd, J = 3.6, 8.4 Hz, 2H), 3.94 (s, 2H), 3.63 (s, 2H), 3.43-3.41 (m, 2H), 2.49-2.45 (m, 2H), 2.26 (s, 3H), 1.61-1.44 (m, 4H) ppm |
| | | 2-(3-Formyl-5- | LCMS (AM3): rt = 0.764 min, (482.2 |
| | | methylphenyl)aceto- | [M + H]⁺), 100.00% purity |
| | | nitrile (Ref: | Purification method 1) PM327 then 2) |
| | | WO2022185041) | PM331 |
| Example 133 | 2-(3-chloro-5- (((4-((1-(6- (pyridazin-4- yl)-1H-indazol- 4-yl)azetidin-3- yl)oxy)butyl)a- mino)methyl)phe- nyl)acetonitrile | 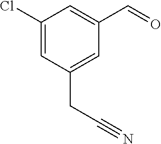 [SMILES: N#CCc1cc(Cl)cc(C=O)c1] | ¹H NMR (400 MHz, DMSO-d₆) δ 9.64 (dd, J = 1.2, 2.4 Hz, 1H), 9.24 (dd, J = 1.2, 5.6 Hz, 1H), 8.09 (s, 1H), 8.01 (dd, J = 2.4, 5.6 Hz, 1H), 7.40 (s, 1H), 7.33-7.24 (m, 3H), 6.30 (d, J = 1.2 Hz, 1H), 4.49-4.40 (m, 3H), 4.05 (s, 2H), 3.97 (dd, J = 3.6, 8.4 Hz, 2H), 3.77 (s, 2H), 3.44 -3.41 (m, 2H), 2.56 (t, J = 6.4 Hz, 2H), 1.63-1.50 (m, 4H). ppm LCMS (AM3): rt = 0.570 min, (502.1 |
| | | 2-(3-chloro-5- | [M + H]⁺), 97.10% purity |
| | | formylphenyl)ace- | Purification Method PM277 |
| | | tonitrile (Ref: | |
| | | WO2022185041) | |
| Example 134 | 2-(3-(((4-((1- (6-(pyridazin- 4-yl)-1H- indazol-4- yl)azetidin-3- yl)oxy)butyl)a- mino)methyl)- 5- (trifluoromethoxy) | 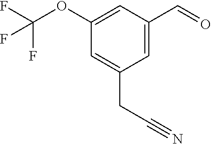 [SMILES: N#CCc1cc(C=O)cc(OC(F)(F)F)c1] | ¹H NMR (400 MHz, DMSO-d₆) δ 9.63 (br s, 1H), 9.24 ( d, J = 5.2 Hz, 1H), 8.14-7.93 (m, 2H), 7.38-7.16 (m, 4H), 6.29 (s, 1H), 4.43 (d, J = 7.6 Hz, 3H), 4.08 (s, 2H), 3.97 (d, J = 4.8 Hz, 2H), 3.72 (s, 2H), 3.44-3.43 (m, 2H), 2.49-2.45 (m, 2H), 1.62-1.43 (m, 4H) ppm LCMS (AM3): rt = 0.600 min, (552.2 [M + H]⁺), 100.00% purity |
| | phenyl)aceto- | 2-(3-Formyl-5- | Purification Method PM281 |
| | nitrile | (trifluoromethoxy) | |
| | | phenyl)acetoni- | |
| | | trile (Ref: | |
| | | WO2022185041) | |

### Example 120

### (3-(((4-((1-(6-(isoxazol-4-yl)-1H-indazol-4-yl)azetidin-3-yl)oxy)butyl)amino)methyl)-5-(trifluoromethyl)phenyl)methanol

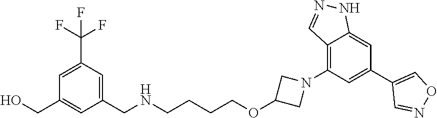 [SMILES: OCc1cc(CNCCCCOC2CN(c3cc(-c4cnoc4)cc4[nH]ncc34)C2)cc(C(F)(F)F)c1]

To a solution of compound 2.115 (300 mg, 803.42 μmol) and 3-(Hydroxymethyl)-5-(trifluoromethyl) benzaldehyde (Ref: WO2022185041, 164.01 mg, 803.42 μmol) in MeOH (2 mL) was added DIPEA (311.50 mg, 2.41 mmol). The mixture was stirred at 20° C. for 12 h, then NaBH(OAc)₃ (681.10 mg, 3.21 mmol) was added and the mixture was stirred at 20° C. for 1 h. The reaction mixture was concentrated in vacuo to give a residue, which was purified (PM205) to afford EXAMPLE 120 (29.53 mg, 52.59 μmol, 6.55% yield, FA salt) as a brown solid.

LCMS (AM3): rt=0.791 min, (516.3 [M+H]⁺), 100% purity

¹H NMR (400 MHz, MeOH-d₄) δ 9.07 (s, 1H), 8.85 (s, 1H), 8.48 (s, 1H), 7.98 (s, 1H), 7.72 (s, 3H), 7.08 (s, 1H), 6.19-6.14 (m, 1H), 4.69 (s, 2H), 4.53-4.48 (m, 1H), 4.46-4.41 (m, 2H), 4.26 (s, 2H), 3.98 (dd, J=4.0, 8.0 Hz, 2H), 3.55 (t, J=5.6 Hz, 2H), 3.10 (t, J=8.0 Hz, 2H), 1.89-1.82 (m, 2H), 1.75-1.70 (m, 2H) ppm

The following examples in Table 24 were made with non-critical changes or substitutions to the exemplified procedure for Example 120, that would be understood by one skilled in the art, using Intermediate 2.115

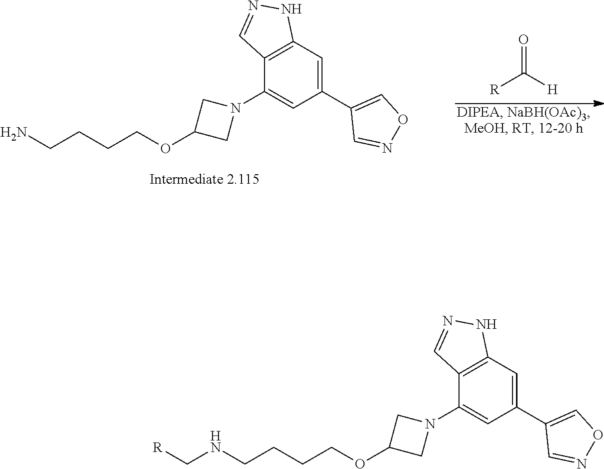

TABLE 24

| | | Aldehyde | |
|---|---|---|---|
| Example | Chemical | (Intermediate | |
| No. | IUPAC name | No./CAS) | Analytical |
| Example 121 | 2-(3-(((4-((1- (6-(isoxazol-4- yl)-1H-indazol- 4-yl)azetidin-3- yl)oxy)butyl)a- mino)methyl)- 5- methylphenyl) acetonitrile | 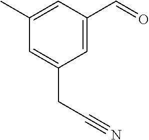 | ¹H NMR (400 MHz, MeOH-d₄) δ 9.08 (s, 1H), 8.86 (s, 1H), 8.51 (s, 1H), 7.98 (d, J = 0.8 Hz, 1H), 7.25 (d, J = 4.4 Hz, 2H), 7.18 (s, 1H), 7.11-7.08 (m, 1H), 6.18 (d, J = 1.2 Hz, 1H), 4.53-4.48 (m, 1H), 4.46-4.41 (m, 2H), 4.14 (s, 2H), 3.95 (dd, J = 4.0, 8.0 Hz, 2H), 3.84 (s, 2H), 3.55 (t, J = 5.6 Hz, 2H), 3.09-3.05 (m, 2H), 2.34 (s, 3H), 1.87- 1.81 (m, 2H), 1.75-1.71 (m, 2H) ppm |
| | | 2-(3-Formyl-5- | LCMS (AM3): rt = 0.788 min, (471.3 |
| | | methylphenyl)ace- | [M + H]⁺), 100% purity |
| | | tonitrile (Ref: | Purification Method PM205 |
| | | WO2022185041) | |
| Example 122 | 2-(3-chloro-5- (((4-((1-(6- (isoxazol-4-yl)- 1H-indazol-4- yl)azetidin-3- yl)oxy)butyl)a- mino)methyl)phe- nyl)acetonitrile | 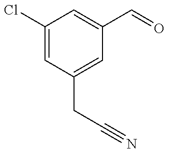 [SMILES: N#CCc1cc(Cl)cc(C=O)c1] | ¹H NMR (400 MHz, MeOH-d₄) δ 9.08 (s, 1H), 8.85 (s, 1H), 8.48 (s, 1H), 7.98 (d, J = 0.8 Hz, 1H), 7.50 (s, 1H), 7.42 (s, 2H), 7.10 (s, 1H), 6.18 (d, J = 1.2 Hz, 1H), 4.53-4.49 (m, 1H), 4.46-4.42 (m, 2H), 4.18 (s, 2H), 3.97 (dd, J = 8.4, 4.0 Hz, 2H), 3.91 (s, 2H), 3.56 (t, J = 6.0 Hz, 2H), 3.08 (t, J = 7.6 Hz, 2H), 1.87-1.83 (m, 2H), 1.75-.72 (m, 2H) ppm LCMS (AM3): rt = 0.799 min, (491.2 |
| | | 2-(3-chloro-5- | [M + H]⁺), 100% purity |
| | | formylphenyl)ace- | Purification Method PM205 |
| | | tonitrile (Ref: | |
| | | WO2022185041) | |
| Example 135 | N-(3-fluoro-4- (trifluoromethoxy) benzyl)-4- ((1-(6- (isoxazol-4-yl)- 1H-indazol-4- yl)azetidin-3- | 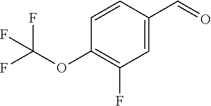 [SMILES: O=Cc1ccc(OC(F)(F)F)c(F)c1] | ¹H NMR (400 MHz, MeOH-d₄) δ 9.07 (s, 1H), 8.85 (s, 1H), 8.46 (s, 1H), 7.99 (d, J = 1.2 Hz, 1H), 7.51-7.46 (m, 2H), 7.37 (d, J = 8.4 Hz, 1H), 7.09 (s, 1H), 6.18 (d, J = 1.2 Hz, 1H), 4.54-4.50 (m, 1H), 4.46-4.43 (m, 2H), 4.20 (s, 2H), 4.00 (dd, J = 8.4, 4.0 Hz, 2H), 3.56 (t, J = 6.0 Hz, 2H), 3.11-3.07 (m, |
| | yl)oxy)butan-1- | CAS 473917- | 2H), 1.87-1.83 (m, 2H), 1.76-1.71 (m, |
| | amine | 15-6 | 2H) ppm |
| | | | LCMS (AM3): rt = 0.820 min, (520.4 |
| | | | [M + H]⁺), 100% purity |
| | | | Purification Method PM79 |
| Example 136 | (3-chloro-5- (((4-((1-(6- (isoxazol-4-yl)- 1H-indazol-4- yl)azetidin-3- yl)oxy)butyl)a- mino)methyl)phe- nyl)methanol | 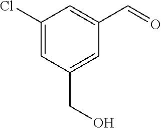 [SMILES: O=Cc1cc(Cl)cc(CO)c1] | ¹H NMR (400 MHz, DMSO-d₆) δ 9.44 (s, 1H), 9.17 (s, 1H), 8.21 (s, 1H), 8.00 (s, 1H), 7.31 (s, 1H), 7.26 (d, J = 4.4 Hz, 2H), 7.21 (s, 1H), 7.09 (s, 1H), 6.19 (s, 1H), 4.48 (s, 2H), 4.45-4.44 (m, 1H), 4.41-4.37 (m, 2H), 3.91 (dd, J = 8.4, 4.0 Hz, 2H), 3.82 (s, 2H), 3.44-3.41 (m, 2H), 2.63 (t, J = 6.4 Hz, 2H), 1.58-1.56 (m, 4H) ppm |
| | | 3-chloro-5- | LCMS (AM3): rt = 0.771 min, (482.3 |
| | | (hydroxymethyl) | [M + H]⁺), 100% purity |
| | | benzaldehyde | Purification Method PM332 |
| | | (Ref: | |
| | | WO2022185041) | |
| Example 138 | (3-fluoro-5- (((4-((1-(6- (isoxazol-4-yl)- 1H-indazol-4- yl)azetidin-3- yl)oxy)butyl)a- mino)methyl)phe- nyl)methanol | 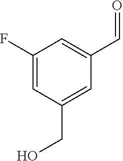 [SMILES: O=Cc1cc(F)cc(CO)c1] | ¹H NMR (400 MHz, DMSO-d₆) δ 9.44 (s, 1H), 9.17 (s, 1H), 8.21 (s, 1H), 8.00 (s, 1H), 7.14 (s, 1H), 7.09-6.99 (m, 4H), 6.19 (s, 1H), 4.49 (s, 2H), 4.47-4.44 (m, 1H), 4.38 (t, J = 7.2 Hz, 2H), 3.91 (dd, J = 4.4, 8.4 Hz, 2H), 3.81 (s, 2H), 3.42 (s, 2H), 2.61 (t, J = 6.8 Hz, 2H), 1.57-1.53 (m, 4H) ppm LCMS (AM3): rt = 0.751 min, (466.3 [M + H]⁺), 100% purity Purification Method PM227 |
| | | 3-Fluoro-5- | |
| | | (hydroxymethyl) | |
| | | benzaldehyde | |
| | | (Ref: | |
| | | WO2022185041 | |
| Example 145 | 2-(3-(((4-((1- (6-(isoxazol-4- yl)-1H-indazol- 4-yl)azetidin-3- yl)oxy)butyl)a- mino)methyl)- 5- (trifluoromethyl) phenyl)acetoni- | 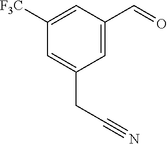 [SMILES: N#CCc1cc(C=O)cc(C(F)(F)F)c1] | ¹H NMR (400 MHz, MeOH-d₄) δ 9.08 (s, 1H), 8.85 (s, 1H), 8.47 (s, 1H), 7.98 (d, J = 0.8 Hz, 1H), 7.81 (s, 1H), 7.76 (s, 1H), 7.73 (s, 1H), 7.09 (s, 1H), 6.18 (d, J = 0.8 Hz, 1H), 4.53-4.50 (m, 1H), 4.46-4.43 (m, 2H), 4.27 (s, 2H), 4.03 (s, 2H), 3.98 (dd, J = 8.4, 4.0 Hz, 2H), 3.56 (t, J = 6.0 Hz, 2H), 3.11-3.07 (m, 2H), 1.87-1.81 (m, 2H), 1.75-1.72 (m, 2H) ppm |
| | trile | 2-(3-Formyl-5- | LCMS (AM3): rt = 0.807 min, (525.3 |
| | | (trifluoromethyl) | [M + H]⁺), 100% purity |
| | | phenyl)acetonitrile | Purification Method PM205 |
| | | (Ref: | |
| | | WO2022185041) | |
| Example 146 | 2-(3-(((4-((1- (6-(isoxazol-4- yl)-1H-indazol- 4-yl)azetidin-3- yl)oxy)butyl)a- mino)methyl)- 5- (trifluoromethoxy) phenyl)aceto- | 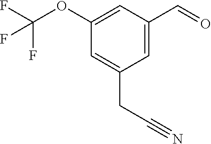 [SMILES: N#CCc1cc(C=O)cc(OC(F)(F)F)c1] | ¹H NMR (400 MHz, MeOH-d₄) δ 9.07 (s, 1H), 8.85 (s, 1H), 8.45 (s, 1H), 7.98 (s, 1H), 7.51 (s, 1H), 7.43 (s, 1H), 7.34 (s, 1H), 7.09 (s, 1H), 6.17 (d, J = 0.8 Hz, 1H), 4.51-4.49 (m, 1H), 4.45-4.41 (m, 2H), 4.24 (s, 2H), 3.99-3.96 (m, 4H), 3.56 (t, J = 6.0 Hz, 2H), 3.10 (t, J = 8.0 Hz, 2H), 1.87-1.83 (m, 2H), 1.75-1.71 (m, 2H) ppm LCMS (AM3): rt = 0.816 min, (541.3 |
| | nitrile | 2-(3-Formyl-5- | [M + H]⁺), 100% purity |
| | | (trifluoromethoxy) | Purification Method PM79 |
| | | phenyl)acetoni- | |
| | | trile (Ref: | |
| | | WO2022185041) | |

### Example 141

### 2-(3-(((4-(2-((6-(4H-1,2,4-triazol-4-yl)-1H-benzo[d][1,2,3]triazol-4-yl)amino)ethoxy)butyl)amino)methyl)-5-(trifluoromethoxy)phenyl)acetonitrile

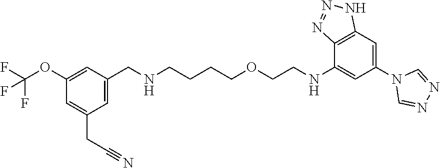 [SMILES: N#CCc1cc(CNCCCCOCCNc2cc(-n3cnnc3)cc3[nH]nnc23)cc(OC(F)(F)F)c1]

To a solution of compound 2.045 (300 mg, 704.65 μmol, HCl salt) and 2-(3-Formyl-5-(trifluoromethoxy)phenyl)acetonitrile (Ref: WO2022185041, 137.25 mg, 598.95 μmol) in DCE (3 mL) and MeOH (0.5 mL) was added DIPEA (273.21 mg, 2.11 mmol, 368.21 μL). The mixture was stirred at 25° C. for 15 h, then NaBH₃CN (88.56 mg, 1.41 mmol) was added and the mixture was stirred at 25° C. for 1 h. The reaction mixture was concentrated in vacuo to give a residue, which was purified (PM333) to afford a crude product. The product was further purified (PM334) to afford EXAMPLE 141 (51.04 mg, 95.81 μmol, 13.60% yield) as a brown gum

LCMS (AM3): rt=0.734 min, (530.1 [M+H]⁺), 99.4% purity

¹H NMR (400 MHz, DMSO-d₆) δ 9.13 (s, 2H), 7.35 (s, 1H), 7.29 (s, 1H), 7.20 (s, 1H), 7.14 (d, J=1.6 Hz, 1H), 6.64 (t, J=5.2 Hz, 1H), 6.58 (d, J=1.6 Hz, 1H), 4.10 (s, 2H), 3.72 (s, 2H), 3.67-3.64 (m, 2H), 3.57 (d, J=4.8 Hz, 4H), 2.49-2.45 (m, 2H), 1.59-1.51 (m, 2H), 1.50-1.43 (m, 2H)

The following examples in Table 25 were made with non-critical changes or substitutions to the exemplified procedure for Example 141, that would be understood by one skilled in the art, using 2-(3-Formyl-5-(trifluoromethoxy)phenyl)acetonitrile (WO2022185041)

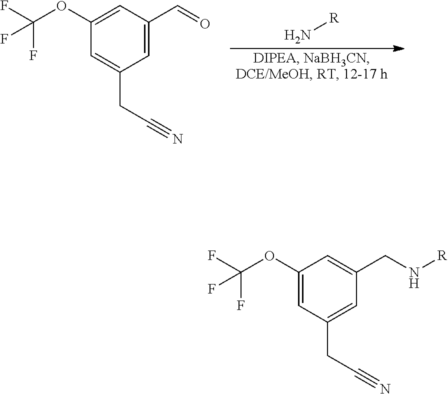

TABLE 25

| Example | Chemical IUPAC | | |
|---|---|---|---|
| No. | name | H₂NR | Analytical |
| Example 148 | 2-(3-(((4-(2-((6- (4H-1,2,4-triazol- 4-yl)-1H- benzo[d][1,2,3]tria- zol-4- yl)oxy)ethoxy)butyl) amino)methyl)- 5- (trifluoromethoxy) phenyl)acetonitrile | 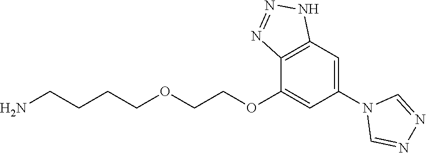 | ¹H NMR (400 MHz, DMSO-d₆) δ 9.17 (s, 2H), 7.69 (d, J = 1.6 Hz, 1H), 7.39 (s, 1H), 7.35 (s, 1H), 7.23 (s, 1H), 7.05 (s, 1H), 4.47 (t, J = 4.0 Hz, 2H), 4.10 (s, 2H), 3.83 (s, 4H), 2.59 (t, J = 6.4 Hz, 2H), 2.53-2.51 (m, 2H), 1.60-1.49 (m, 4H) ppm LCMS (AM3): rt = 0.756 min, (531.3 [M + H]⁺), 100.00% purity Purification Method PM335 |
| | | (Intermediate 2.347) | |
| Example 151 | 2-(3-(((4-((1-(6- (pyridazin-4-yl)- 1H- benzo[d][1,2,3]tria- zol-4-yl)azetidin- 3- yl)oxy)butyl)amino) methyl)-5- (trifluoromethoxy) phenyl)acetonitrile | 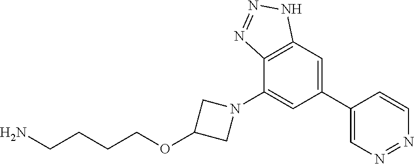 | ¹H NMR (400 MHz, DMSO-d₆) δ 9.68 (dd, J = 1.2, 2.4 Hz, 1H), 9.27 (dd, J = 1.2, 5.6 Hz, 1H), 8.06 (dd, J = 2.4, 5.6 Hz, 1H), 7.48 (d, J = 1.2 Hz, 1H), 7.38 (s, 1H), 7.32 (s, 1H), 7.20 (s, 1H), 6.52 (d, J = 1.2 Hz, 1H), 4.58-4.52 (m, 2H), 4.52-4.47 (m, 1H), 4.11 (s, 2H), 4.10-4.06 (m, 2H), 3.77 (s, 2H), 3.44 (t, J = 6.0 Hz, 2H), 2.55-2.52 (m, 2H), |
| | | (Intermediate 2.285) | 1.67-1.44 (m, 4H) ppm |
| | | | LCMS (AM3): rt = 0.747 min, |
| | | | (553.4 [M + H]⁺), 99.19% purity |
| | | | Purification Method PM225 |

### Example 154

### 2-(3-(((4-((1-(6-(4H-1,2,4-triazol-4-yl)-1H-benzo[d][1,2,3]triazol-4-yl)azetidin-3-yl)oxy)butyl)amino)methyl)-5-(trifluoromethoxy)phenyl)acetonitrile

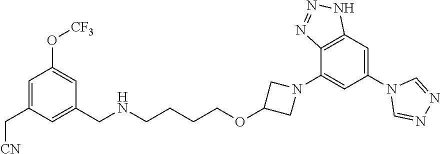 [SMILES: N#CCc1cc(CNCCCCOC2CN(c3cc(-n4cnnc4)cc4[nH]nnc34)C2)cc(OC(F)(F)F)c1]

A mixture of 2-(3-Formyl-5-(trifluoromethoxy)phenyl)acetonitrile (Ref: WO2022185041, 108 mg, 471.30 μmol), compound 2.304 (160 mg, 427.35 μmol, FA salt) and DIPEA (0.15 mL, 861.18 μmol) in MeOH (10 mL) was stirred at 25° C. for 16 h before NaBH(OAc)₃ (362 mg, 1.71 mmol) was added. The reaction mixture was stirred for 1 h. The reaction mixture was concentrated in vacuo to give a residue, which was purified (PM336) to afford EXAMPLE 154 (33.18 mg, 14.07% yield) as a brown solid.

LCMS (AM3): rt=0.789 min, (542.2 [M+H]⁺), 98.11% purity.

¹H NMR (400 MHz, DMSO-d₆) δ 9.15 (s, 2H), 7.37 (s, 1H), 7.31 (s, 1H), 7.23 (s, 1H), 7.20 (s, 1H), 6.35 (s, 1H), 4.59-4.48 (m, 3H), 4.12-4.05 (m, 4H), 3.75 (s, 2H), 3.43 (t, J=5.6 Hz, 2H), 2.52-2.50 (m, 2H), 1.66-1.55 (m, 2H), 1.55-1.45 (m, 2H) ppm

### Example 155

### 2-(3-(((4-(2-((6-(pyridazin-4-yl)-1H-indazol-4-yl)oxy)ethoxy)butyl)amino)methyl)-5-(trifluoromethyl)phenyl)acetonitrile

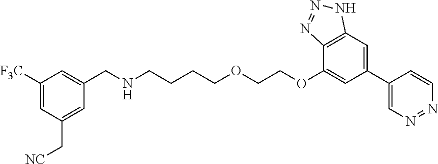 [SMILES: N#CCc1cc(CNCCCCOCCOc2cc(-c3ccnnc3)cc3[nH]nnc23)cc(C(F)(F)F)c1]

To a solution of compound 2.367 (71.61 mg, 312.47 μmol) in MeOH (2 mL) was added DIPEA (85.02 mg, 657.84 μmol, 114.58 μL) and 2-(3-Formyl-5-(trifluoromethyl)phenyl)acetonitrile (Ref: WO2022185041, 120 mg, 328.92 μmol). The reaction was stirred at 20° C. for 12 h before NaBH(OAc)₃ (348.56 mg, 1.64 mmol) was added. The reaction was stirred at 25° C. for 2 h. The mixture was concentrated in vacuo to give a residue, which was purified (PM337) to afford EXAMPLE 155 (63.63 mg, 116.33 μmol, 35.37% yield, 99% purity) as a yellow solid.

LCMS (AM3): rt=0.761 min, (542.5 [M+H]⁺), 99.30% purity

¹H NMR (400 MHz, MeOH-d₄) δ 9.73-9.72 (m, 1H), 9.24-9.23 (m, 1H), 8.09-8.07 (m, 1H), 7.97 (s, 1H), 7.37 (s, 1H), 7.32 (s, 1H), 7.20-7.18 (m, 2H), 4.51 (t, J=4.8 Hz, 2H), 4.09 (s, 2H), 3.82 (t, J=4.4 Hz, 2H), 3.62 (s, 2H), 3.50 (t, J=6.0 Hz, 2H), 2.55-2.50 (m, 2H), 1.59-1.52 (m, 4H) ppm

### Example 156

### 3-((3-chloro-4-(trifluoromethoxy)benzyl)amino)-N-(3-((6-(5-cyano-1H-pyrazol-4-yl)-1H-indazol-4-yl)amino)propyl)propanamide

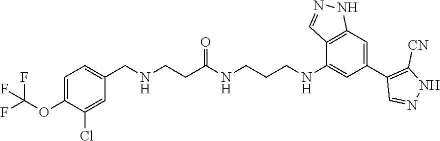 [SMILES: N#Cc1[nH]ncc1-c1cc(NCCCNC(=O)CCNCc2ccc(OC(F)(F)F)c(Cl)c2)c2cn[nH]c2c1]

To a solution of compound 2.226 (100 mg, 120.58 μmol) in DCM (5 mL) was added TFA (1 mL, 13.51 mmol) at 20° C. and the mixture was stirred for 2 h. Sat. NaHCO₃ (aq.) (40 mL) was added and the mixture was extracted with EA (20 mL×3). The combined organic phase was washed (brine, 15 mL×2), dried (Na₂SO₄), filtered and concentrated in vacuo to give a residue, which was purified (PM235) to afford EXAMPLE 156 (4.55 mg, 8.07 μmol, 6.37% yield) as a yellow solid.

LCMS (AM16): rt=0.603 min, (561.2 [M+H]⁺), 99.53% purity.

¹H NMR (400 MHz, MeOH-d₄) δ 8.14 (d, J=6.4 Hz, 2H), 7.53 (s, 1H), 7.31 (s, 2H), 7.07 (s, 1H), 6.45 (s, 1H), 3.73 (s, 2H), 3.37 (t, J=6.4 Hz, 4H), 2.83 (t, J=6.4 Hz, 2H), 2.42 (t, J=6.8 Hz, 2H), 2.03-1.84 (m, 2H).

### Example 158

### 3-((3-chloro-4-(trifluoromethoxy)benzyl)amino)-N-(3-((7-chloro-6-(pyridin-4-yl)-1H-indazol-4-yl)amino)propyl)propanamide

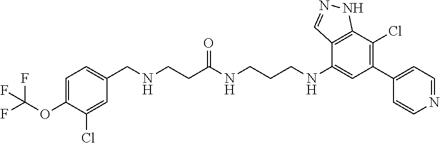 [SMILES: O=C(CCNCc1ccc(OC(F)(F)F)c(Cl)c1)NCCCNc1cc(-c2ccncc2)c(Cl)c2[nH]ncc12]

To a solution of compound 2.249 (60 mg, 78.37 μmol) in DCM (3 mL) was added TFA (0.3 mL, 4.05 mmol). The mixture was stirred at 25° C. for 1 h. The reaction mixture was added to saturated sodium bicarbonate aqueous solution (10 mL) until pH=9 and extracted with EA (10 mL×3). The combined organic layers were washed (brine, 15 mL×2), dried (Na₂SO₄), filtered and concentrated in vacuo to give a residue, which was purified (PM338) to afford EXAMPLE 158 (16.62 mg, 28.19 μmol, 35.97% yield) as a white solid.

LCMS (AM11): rt=0.314 min, (581.1 [M+H]⁺), 98.61% purity.

¹H NMR (400 MHz, MeOH-d₄) δ 8.62-8.57 (m, 2H), 8.24 (s, 1H), 7.60-7.50 (m, 3H), 7.31 (s, 2H), 6.12 (s, 1H), 3.72 (s, 2H), 3.36-3.31 (m, 4H), 2.80 (t, J=6.4 Hz, 2H), 2.38 (t, J=6.4 Hz, 2H), 1.95-1.86 (m, 2H) ppm.

### Example 159

### 3-((3-chloro-4-(trifluoromethoxy)benzyl)amino)-N-(3-((6-(5-methyl-1H-pyrazol-4-yl)-1H-indazol-4-yl)amino)propyl)propanamide

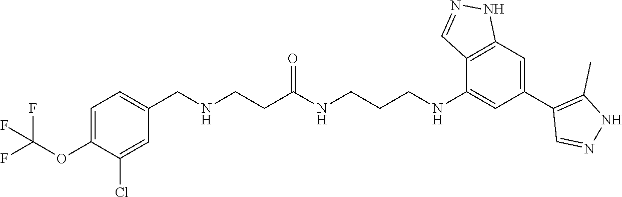 [SMILES: Cc1[nH]ncc1-c1cc(NCCCNC(=O)CCNCc2ccc(OC(F)(F)F)c(Cl)c2)c2cn[nH]c2c1]

To a solution of compound 2.252 (30 mg, 36.66 μmol) in DCM (3 mL) was added TFA (0.3 mL, 4.05 mmol) and the mixture was stirred at 25° C. for 1 hour. The reaction mixture was added to sat. NaHCO₃ (aq.) (10 mL) until pH=9 and extracted with EA (10 mL×3). The combined organic layers were washed (brine, 15 mL×2), dried (Na₂SO₄), filtered and concentrated in vacuo to give a residue, which was purified (PM237) to afford EXAMPLE 159 (8.22 mg, 14.30 μmol, 39.00% yield) as a yellow gum.

LCMS (AM11): rt=0.330 min, (550.2 [M+H]⁺), 95.66% purity.

¹H NMR (400 MHz, CD₃OD) δ 8.10 (s, 1H), 7.70 (s, 1H), 7.52 (s, 1H), 7.30 (s, 2H), 6.78 (s, 1H), 6.23 (s, 1H), 3.71 (s, 2H), 3.40-3.33 (m, 4H), 2.81 (t, J=6.8 Hz, 2H), 2.44 (s, 3H), 2.41-2.38 (m, 2H), 1.99-1.86 (m, 2H) ppm.

### Example 163

### N-(3-((1H-indazol-4-yl)amino)propyl)-3-((3-chloro-4-(trifluoromethoxy)benzyl)amino)propanamide

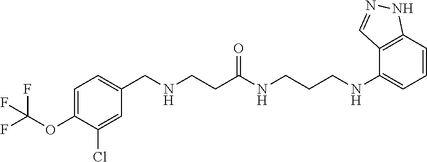 [SMILES: O=C(CCNCc1ccc(OC(F)(F)F)c(Cl)c1)NCCCNc1cccc2[nH]ncc12]

To a solution of compound 2.390 (28 mg, 49.12 μmol) in DCM (10 mL) was added TFA (1 mL, 13.51 mmol). The mixture was stirred at 20° C. for 1 h. The reaction mixture was poured into saturated NaHCO₃ (20 mL) solution and extracted with EA (20 mL×3). The combined organic phase was washed (brine, 20 mL×3), dried (Na₂SO₄), filtered and concentrated in vacuo to give a residue, which was purified (PM339) to afford EXAMPLE 163 (4.63 mg, 9.34 μmol, 19.02% yield, 94.8% purity) as a yellow gum.

LCMS (AM12): rt=0.508 min, (470.2, [M+H]⁺), 94.81% purity

¹H NMR (400 MHz, MeOH-d₄) δ 8.11 (s, 1H), 7.55 (s, 1H), 7.33 (s, 2H), 7.14 (t, J=8.0 Hz, 1H), 6.74 (d, J=8.0 Hz, 1H), 6.15 (d, J=8.0 Hz, 1H), 3.75 (s, 2H), 3.37-3.32 (m, 4H), 2.83 (t, J=7.6 Hz, 2H), 2.41 (t, J=6.4 Hz, 2H), 1.95-1.86 (m, 2H) ppm

The following examples in Table 26 were made with non-critical changes or substitutions to the exemplified procedure for Example 163, that would be understood by one skilled in the art.

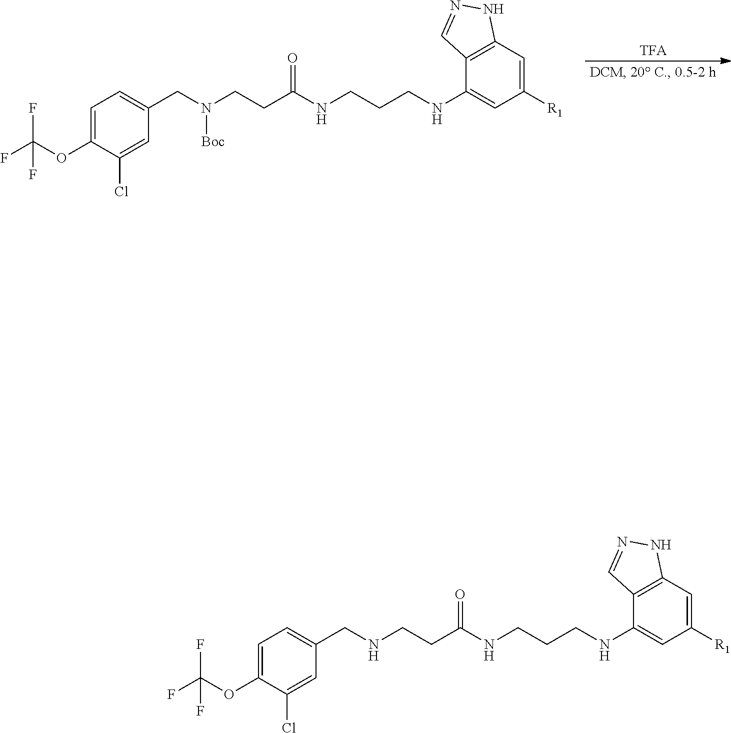

TABLE 26

| Example | Chemical | R₁ | |
|---|---|---|---|
| No. | IUPAC name | (Intermediate) | Analytical |
| Example 164 | 3-((3-chloro-4- (trifluoromethoxy) benzyl)ami- no)-N-(3-((6- | 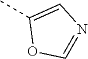 | ¹H NMR (400 MHz, DMSO-d₆) δ 12.87 (br s, 1H), 8.88 (br. s, 2H), 8.40 (s, 1H), 8.22 (t, J = 5.6 Hz, 1H), 8.18 (s, 1H), 7.86 (d, J = 1.6 Hz, 1H), 7.70-7.62 (m, 2H), 7.60 (dd, J = |
| | (oxazol-5-yl)- | (Intermediate | 8.8, 1.6 Hz, 1H), 7.01 (s, 1H), 6.46-6.26 |
| | 1H-indazol-4- | 2.389) | (m, 2H), 4.21 (s, 2H), 3.33-3.20 (m, 4H), |
| | yl)amino)propyl) | | 3.16-3.06 (m, 2H), 2.56-2.53 (m, 2H), |
| | propanamide | | 1.86-1.76 (m, 2H) ppm |
| | | | LCMS (AM12): rt = 0.438 min, (537.3 |
| | | | [M + H]⁺), 99.58% purity |
| | | | Purification Method PM340 |
| Example 165 | 3-((3-chloro-4- (trifluoromethoxy) benzyl)ami- no)-N-(3-((6- (3- methylpyridin- 4-yl)-1H- | 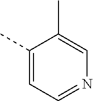 | ¹H NMR (400 MHz, MeOH-d₄) δ 8.42 (s, 1H), 8.36 (d, J = 4.8 Hz, 1H), 8.18 (s, 1H), 7.54 (s, 1H), 7.35-7.26 (m, 3H), 6.68 (s, 1H), 6.06 (s, 1H), 3.73 (s, 2H), 3.38-3.33 (m, 4H), 2.81 (t, J = 6.8 Hz, 2H), 2.38 (t, J = 6.8 Hz, 2H), 2.31 (s, 3H), 1.95-1.86 (m, 2H) ppm |
| | indazol-4- | (Intermediate | LCMS (AM16): rt = 0.665 min, (561.2 |
| | yl)amino)propyl) | 2.393) | [M + H]⁺), 97.20% purity. |
| | propanamide | | Purification Method PM341 |

### Example 166

### 7-chloro-N-(2-(4-((3,5-difluoro-4-(trifluoromethoxy)benzyl)amino)butoxy)ethyl)-6-(pyridin-4-yl)-1H-indazol-4-amine

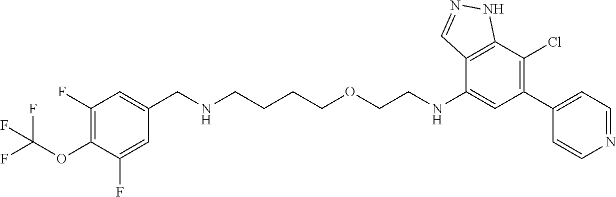 [SMILES: Fc1cc(CNCCCCOCCNc2cc(-c3ccncc3)c(Cl)c3[nH]ncc23)cc(F)c1OC(F)(F)F]

To a solution of compound 2.255 (48 mg, 63.64 μmol) in DCM (3 mL) was added TFA (0.6 mL, 8.10 mmol). The mixture was stirred at 20° C. for 1 h. The reaction mixture was poured into saturated NaHCO₃ aqueous solution (30 mL) and extracted with EA (30 mL×3). The combined organic phases were washed by brine (30 mL×2), dried (Na₂SO₄), filtered and concentrated in vacuo to give a residue, which was purified (PM342) to afford EXAMPLE 166 (20.71 mg, 35.12 μmol, 55.19% yield) as a white solid.

LCMS (AM11): rt=0.319 min, (570.1 [M+H]⁺), 96.66% purity.

¹H NMR (400 MHz, MeOH-d₄) δ 8.63 (d, J=6.0 Hz, 2H), 8.25 (s, 1H), 7.60-7.58 (m, 2H), 7.19 (d, J=8.4 Hz, 2H), 6.20 (s, 1H), 3.79 (s, 2H), 3.72 (t, J=5.6 Hz, 2H), 3.55 (t, J=5.6 Hz, 2H), 3.48 (t, J=5.6 Hz, 2H), 2.67 (t, J=5.6 Hz, 2H), 1.78-1.59 (m, 4H) ppm.

### Example 167

### 4-((2-(4-((3,5-difluoro-4-(trifluoromethoxy)benzyl)amino)butoxy)ethyl)amino)-1H-indazole-6-carbonitrile

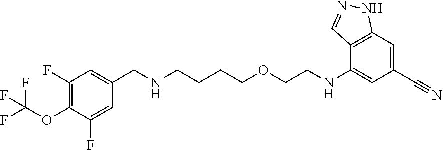 [SMILES: N#Cc1cc(NCCOCCCCNCc2cc(F)c(OC(F)(F)F)c(F)c2)c2cn[nH]c2c1]

To a mixture of compound 2.248 (30 mg, 44.93 μmol) in DCM (1 mL) was added TFA (0.3 mL, 4.05 mmol). The mixture was stirred at 25° C. for 1 h. The mixture was concentrated in vacuo to give a residue, which was purified (PM343) to afford EXAMPLE 167 (17.59 mg, 35.81 μmol, 79.70% yield) as an off-white solid.

LCMS (AM12): rt=0.458 min, (484.3 [M+H]⁺), 98.42% purity.

¹H NMR (400 MHz, MeOH-d₄) δ 8.22 (s, 1H), 7.17 (s, 1H), 7.14 (d, J=3.6 Hz, 2H), 6.37 (s, 1H), 3.72-3.69 (m, 4H), 3.53 (t, J=6.4 Hz, 2H), 3.48 (t, J=5.4 Hz, 2H), 2.58 (t, J=6.4 Hz, 2H), 1.65-1.60 (m, 4H) ppm.

The following examples in Table 27 were made with non-critical changes or substitutions to the exemplified procedure for Example 167, that would be understood by one skilled in the art

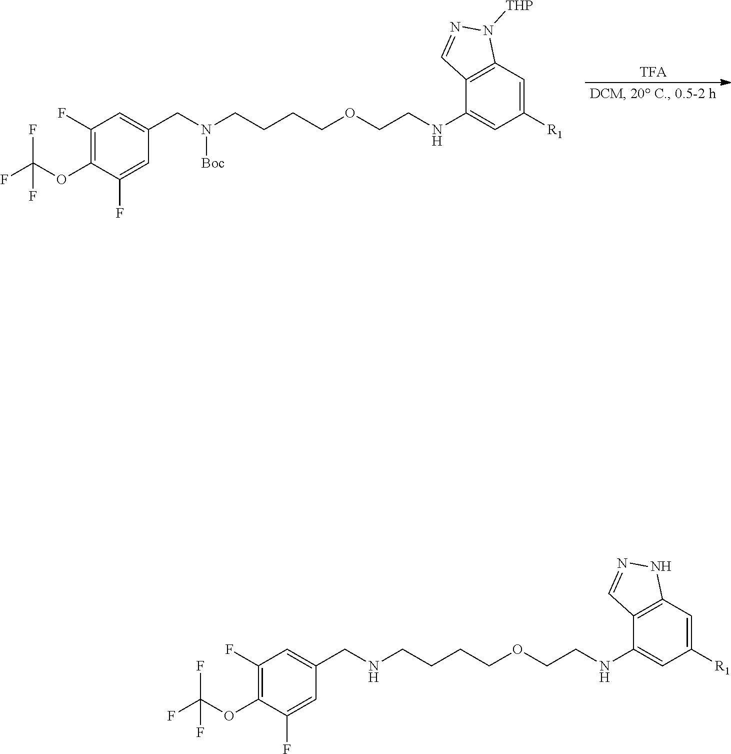

TABLE 27

| Example | Chemical | R₁ | |
|---|---|---|---|
| No. | IUPAC name | (Intermediate) | Analytical |
| Example 171 | 4-(2-(((6-(4H- 1,2,4-triazol-4- yl)-1H-indazol- 4- yl)methyl)amino) | 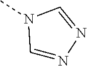 | ¹H NMR (400 MHz, MeOH-d₄) δ 9.05 (s, 2H), 8.33 (s, 1H), 7.73 (s, 1H), 7.40 (s, 1H), 7.18 (d, J = 8.8 Hz, 2H), 4.22 (s, 2H), 3.75 (s, 2H), 3.57 (t, J = 4.0 Hz, 2H), 3.46 (t, J = 4.0 Hz, 2H), 2.85 (t, J = 5.2 Hz, 2H), |
| | ethoxy)-N- | (Intermediate | 2.57 (t, J = 7.2 Hz, 2H), 1.62-1.56 (m, 4H) |
| | (3,5-difluoro-4- | 2.490) | ppm |
| | (trifluoromethoxy) | | LCMS (AM11): rt = 0.311 min, (540.2 |
| | benzyl)butan- | | [M + H]⁺), 99.51% purity |
| | 1-amine | | Purification Method PM344 |
| Example 198 | 2-(4-((2-(4- ((3,5-difluoro- 4- (trifluoromethoxy) benzyl)ami- | 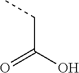 | ¹H NMR (400 MHz, DMSO-d₆) δ 12.68- 12.56 (m, 1 H), 12.37-12.00 (m, 1H), 8.81- 8.73 (m, 2 H), 8.09 (s, 1 H), 7.54 (br d, J = 8.8 Hz, 2 H), 6.57 (s, 1 H), 6.22-6.06 (m, 1 H), 5.96 (s, 1 H), 4.16 (br s, 2 H), 3.62 (br t, |
| | no)butoxy)ethyl) | (Intermediate | J = 6.0 Hz, 2 H), 3.51 (s, 2 H), 3.46 (t, J = |
| | amino)-1H- | 1.3) | 6.0 Hz, 2 H), 2.99-2.93 (m,, 1 H), 2.62- |
| | indazol-6- | | 2.58 (m, 2H), 1.70-1.64 (m, 2 H), 1.62- |
| | yl)acetic acid | | 1.57 (m, 2 H) ppm |
| | | | LCMS (AM11): rt = 0.374 min, (517.1 |
| | | | [M + H]⁺), 9.05% purity |
| | | | Purification method 1) PM246 then 2) |
| | | | RM244 |

### Example 172

### N-(3-(((6-(4H-1,2,4-triazol-4-yl)-1H-indazol-4-yl)methyl)amino)propyl)-3-((3-chloro-4-(trifluoromethoxy)benzyl)amino)propanamide

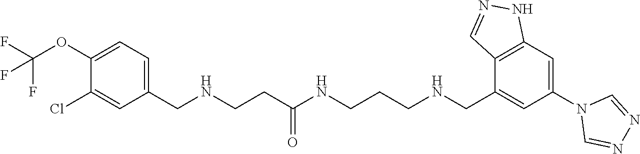 [SMILES: O=C(CCNCc1ccc(OC(F)(F)F)c(Cl)c1)NCCCNCc1cc(-n2cnnc2)cc2[nH]ncc12]

To a solution of compound 2.489 (70 mg, 95.21 μmol) in DCM (5 mL) was added TFA (1 mL, 13.51 mmol). The mixture was stirred at 20° C. for 1 hour. The mixture was basified to pH=8 by saturated NaHCO₃ aqueous solution and then concentrated in vacuo to give a residue, which was purified (PM248) to afford EXAMPLE 172 (20.15 mg, 36.08 μmol, 37.90% yield) as a yellow gum.

LCMS (AM16): rt=0.556 min, (551.2, [M+H]⁺), 98.66% purity

¹H NMR (400 MHz, MeOH-d₄) δ 9.06 (s, 2H), 8.35 (d, J=0.4 Hz, 1H), 7.73 (s, 1H), 7.56 (s, 1H), 7.40 (d, J=1.2 Hz, 1H), 7.35 (s, 2H), 4.18 (s, 2H), 3.75 (s, 2H), 3.26 (t, J=6.8 Hz, 2H), 2.80 (t, J=6.8 Hz, 2H), 2.72 (t, J=6.8 Hz, 2H), 2.37 (t, J=6.8 Hz, 2H), 1.76 (m, 2H) ppm

### Example 173

### N-(3-((6-(1,2,4-oxadiazol-5-yl)-1H-indazol-4-yl)amino)propyl)-3-((3-chloro-4-(trifluoromethoxy)benzyl)amino)propanamide

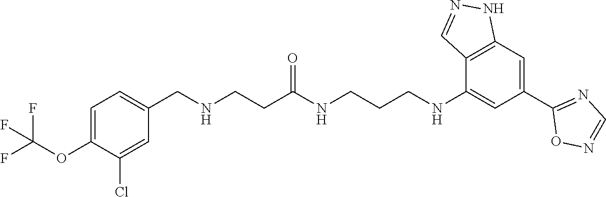 [SMILES: O=C(CCNCc1ccc(OC(F)(F)F)c(Cl)c1)NCCCNc1cc(-c2ncno2)cc2[nH]ncc12]

To a solution of compound −0 (35 mg, 44.98 μmol, 82% purity) in DCM (2 mL) was added TFA (0.4 mL, 5.40 mmol). The mixture was stirred at 20° C. for 0.5 h. The mixture was concentrated in vacuo to give a residue, which was purified (PM345) to afford EXAMPLE 173 (6.19 mg, 11.08 μmol, 24.63% yield) as a white solid.

LCMS (AM12): rt=0.468 min, (538.2 [M+H]⁺), 96.28% purity.

¹H NMR (400 MHz, MeOH-d₄) δ 8.72 (s, 1H), 8.23 (s, 1H), 7.59 (s, 1H), 7.54 (s, 1H), 7.31 (d, J=2.8 Hz, 2H), 6.84 (s, 1H), 3.77 (s, 2H), 3.41-3.37 (m, 4H), 2.86 (t, J=6.8 Hz, 2H), 2.45 (t, J=6.8 Hz, 2H), 1.96 (t, J=6.8 Hz, 2H) ppm.

The following examples in Table 28 were made with non-critical changes or substitutions to the exemplified procedure for Example 173, that would be understood by one skilled in the art

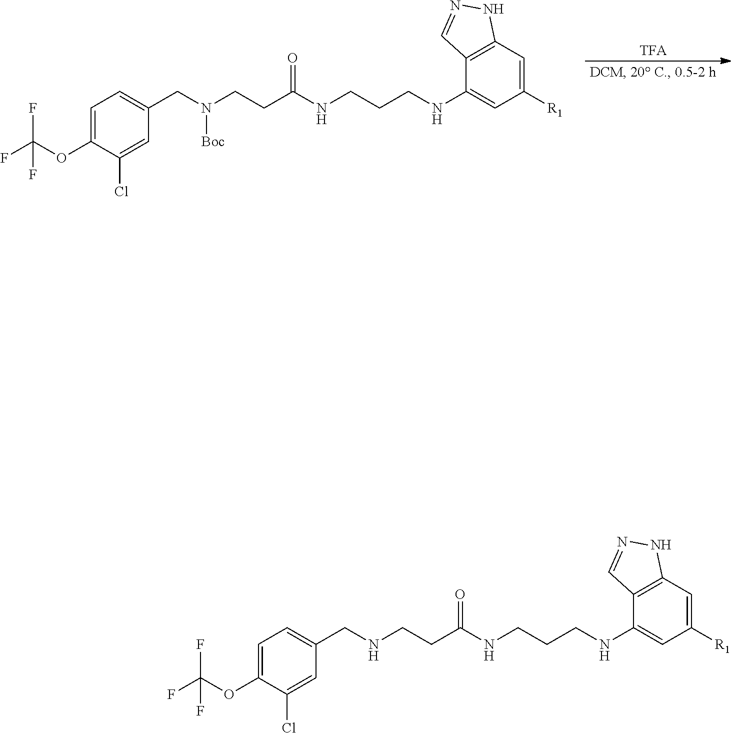

TABLE 28

| Example | Chemical | R₁ | |
|---|---|---|---|
| No. | IUPAC name | (Intermediate) | Analytical |
| Example 175 | 3-((3-chloro-4- (trifluoromethoxy) benzyl)ami- no)-N-(3-((6- | 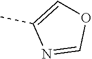 | ¹H NMR (400 MHz, MeOH-d₄) δ 8.31 (s, 1H), 8.26 (s, 1H), 8.11 (s, 1H), 7.75 (s, 1H), 7.53 (s, 2H), 7.20 (s, 1H), 6.55 (s, 1H), 4.25 (s, 2H), 3.40 (q, J = 6.8 Hz, 6H), 2.67 (t, J = |
| | (oxazol-4-yl)- | (Intermediate | 6.4 Hz, 2H), 1.96 (quin, J = 6.8 Hz, 2H) |
| | 1H-indazol-4- | 2.341) | ppm |
| | yl)amino)propyl) | | LCMS (AM14): rt = 0.562 min, (537.3, |
| | propanamide | | [M + H]⁺), 96.4% purity |
| | | | Purification Method PM346 |
| Example 184 | N-(3-((6-(2- aminopyridin- 4-yl)-1H- indazol-4- yl)amino)propyl)- 3-((3-chloro- 4- | 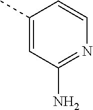 | ¹H NMR (400 MHz, MeOH-d₄) δ 8.17 (s, 1H), 7.93 (d, J = 5.2 Hz, 1H), 7.54 (s, 1H), 7.32 (s, 2H), 7.00 (s, 1H), 6.92 (dd, J = 6.4, 1.6 Hz, 1H), 6.88 (s, 1H), 6.38 (s, 1H), 3.73 (s, 2H), 3.42-3.37 (m, 4H), 2.83 (t, J = 6.8, 2H), 2.43 (t, J = 6.4 Hz, 2H), 1.97 (t, J = 6.8 Hz, 2H) ppm LCMS (AM11): rt = 0.340 |
| | (trifluoromethoxy) | (Intermediate | min, (562.2 [M + H]⁺), 100% purity |
| | benzyl)ami- | 2.323) | Purification Method PM347 |
| | no)propanamide | | |

### Example 176

### N-(2-(4-((3,5-difluoro-4-(trifluoromethoxy)benzyl)amino)butoxy)ethyl)-7-fluoro-6-(pyridin-4-yl)-1H-indazol-4-amine

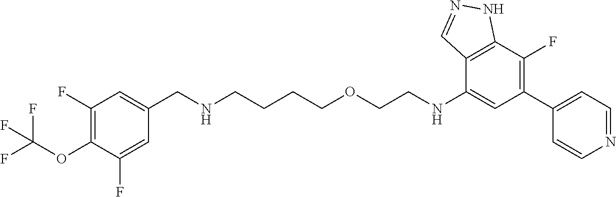 [SMILES: Fc1cc(CNCCCCOCCNc2cc(-c3ccncc3)c(F)c3[nH]ncc23)cc(F)c1OC(F)(F)F]

To a solution of compound 2.264 (105 mg, 142.33 μmol) in DCM (6 mL) was added TFA (1 ml-, 13.51 mmol) at 20° C. The mixture was stirred at 20° C. for 1 h. The reaction mixture was concentrated in vacuo below 30° C. The residue was diluted with MeOH (3 mL) and basified to pH=10 by NH₃·H₂O, then the resulting mixture was purified (PM348) to afford EXAMPLE 176 (24.02 mg, 41.86 μmol, 29.41% yield) as a grey solid.

LCMS (AM12): rt=0.397 min, (554.3 [M+H]⁺), 96.47% purity.

¹H NMR (400 MHz, MeOH-d₄) δ 8.62-8.57 (m, 2H), 8.22 (d, J=3.2 Hz, 1H), 7.69 (d, J=4.8 Hz, 2H), 7.16-7.13 (m, 2H), 6.18 (d, J=4.4 Hz, 1H), 3.73 (t, J=5.6 Hz, 2H), 3.70 (s, 2H), 3.53 (t, J=6.0 Hz, 2H), 3.48 (t, J=5.6 Hz, 2H), 2.56 (t, J=6.8 Hz, 2H), 1.66-1.56 (m, 4H) ppm.

### Example 178

### 3-((3-chloro-4-(trifluoromethoxy)benzyl)amino)-N-(3-((7-methyl-6-(pyridin-4-yl)-1H-indazol-4-yl)amino)propyl)propanamide

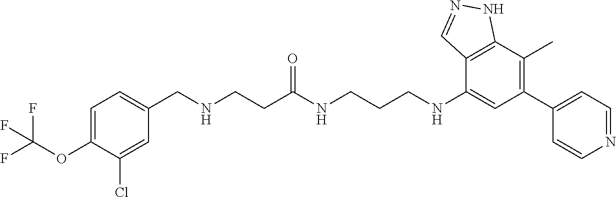 [SMILES: Cc1c(-c2ccncc2)cc(NCCCNC(=O)CCNCc2ccc(OC(F)(F)F)c(Cl)c2)c2cn[nH]c12]

To a solution of compound 2.026 (50 mg, 58.21 μmol, 86.753% purity) in DCM (1 mL) was added TFA (308.00 mg, 2.70 mmol, 0.2 mL) dropwise. The mixture was stirred at 20° C. for 1 h. The mixture was concentrated in vacuo and adjusted to pH=8 with DIPEA. The residue was purified (PM349) and (PM350) to afford EXAMPLE 178 (4.76 mg, 8.38 μmol, 14.40% yield) as a yellow solid.

LCMS (AM3): rt=0.750 min, (561.2 [M+H]⁺), 98.752% purity.

¹H NMR (400 MHz, MeOH-d₄): δ 8.57 (d, J=6.0 Hz, 2H), 8.43 (s, 1H), 8.17 (s, 1H), 7.67 (s, 1H), 7.46-7.45 (m, 1H), 7.44 (s, 2H), 6.04 (s, 1H), 4.04 (s, 2H), 3.77-3.33 (m, 2H), 3.29-3.27 (m, 2H), 3.09 (t, J=6.4 Hz, 2H), 2.54 (t, J=6.4 Hz, 2H), 2.31 (s, 3H), 1.90 (t, J=6.4 Hz, 2H) ppm.

¹⁹F NMR (400 MHz, MeOH-d₄): δ −59.571 ppm

The following examples in Table 29 were made with non-critical changes or substitutions to the exemplified procedure for Example 178, that would be understood by one skilled in the art

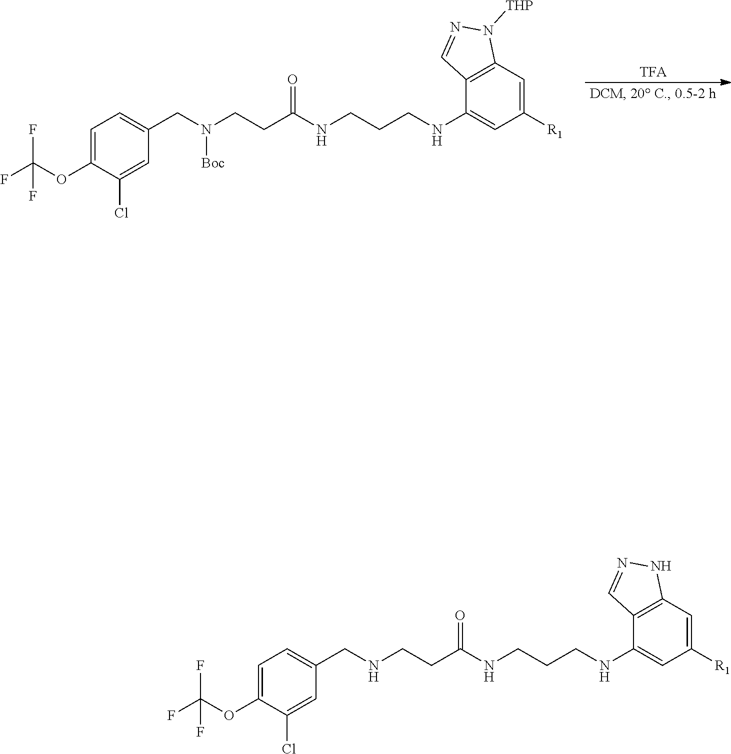

TABLE 29

| Example | Chemical | R1 | |
|---|---|---|---|
| No. | IUPAC name | (Intermediate) | Analytical |
| Example 188 | N-(3-((6-(6- aminopyridazin- 4-yl)-1H-indazol- 4- yl)amino)propyl)- 3-((3-chloro-4- (trifluoromethoxy) | 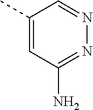 | ¹H NMR (400 MHz, MeOH-d₄) δ 8.73 (d, J = 1.6 Hz, 1H), 8.18 (s, 1H), 7.53 (s, 1H), 7.30 (s, 2H), 7.17 (s, 1H), 7.05 (s, 1H), 6.35 (s, 1H), 3.74 (s, 2H), 3.41-3.33 (m, 4H), 2.83 (t, J = 6.8 Hz, 2H), 2.42 (t, J = 6.8 Hz, 2H), 1.95 (t, J = 6.8 Hz, 2H) ppm LCMS (AM3): rt = 0.733 min, (563.1 [M + H]⁺), |
| | benzyl)amino)pro- | (Intermediate | 99.725% purity. |
| | panamide | 2.027) | Purification Method PM351 |

### Example 179

### N-(2-(4-((3,5-difluoro-4-(trifluoromethoxy)benzyl)amino)butoxy)ethyl)-7-methyl-6-(pyridin-4-yl)-1H-indazol-4-amine

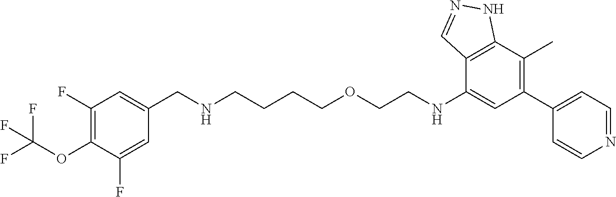 [SMILES: Cc1c(-c2ccncc2)cc(NCCOCCCCNCc2cc(F)c(OC(F)(F)F)c(F)c2)c2cn[nH]c12]

To a solution of compound 2.382 (70 mg, 95.40 μmol) in DCM (1 mL) was added TFA (308.00 mg, 2.70 mmol, 0.2 mL). The reaction was stirred at 20° C. for 1 h. The mixture was concentrated in vacuo to give a residue, which was purified (PM352) to afford EXAMPLE 179 (21.84 mg, 38.16 μmol, 40.00% yield) as a yellow solid.

LCMS (AM3): rt=0.547 min, (550.1 [M+H]⁺), 96.02% purity.

¹H NMR (400 MHz, MeOH-d₄) δ8.58 (dd, J=4.8, 1.6 Hz, 2H), 8.16 (s, 1H), 7.46 (dd, J=4.8, 1.6 Hz, 2H), 7.19 (d, J=8.4 Hz, 2H), 6.10 (s, 1H), 3.88 (s, 2H), 3.72 (t, J=5.2 Hz, 2H), 3.55 (t, J=5.6 Hz, 2H), 3.44 (t, J=5.2 Hz, 2H), 2.81 (t, J=6.4 Hz, 2H), 2.32 (s, 3H), 1.69-1.67 (m, 4H) ppm.

### Example 180

### 3-((3-chloro-4-(trifluoromethoxy)benzyl)amino)-N-(3-((7-fluoro-6-(pyridin-4-yl)-1H-indazol-4-yl)amino)propyl)propanamide

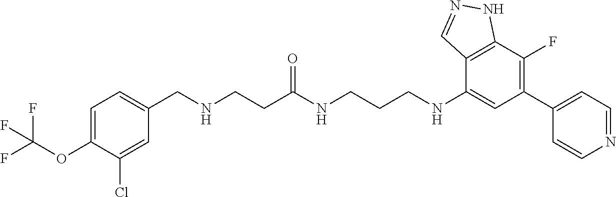 [SMILES: O=C(CCNCc1ccc(OC(F)(F)F)c(Cl)c1)NCCCNc1cc(-c2ccncc2)c(F)c2[nH]ncc12]

To a solution of compound 2.320 (60 mg, 80.09 μmol) in DCM (5 mL) was added TFA (1 mL, 13.51 mmol). The mixture was stirred at 20° C. for 2 h. The reaction mixture was concentrated in vacuo to give a residue, which was purified (PM353) to afford EXAMPLE 180 (19.01 mg, 33.65 μmol, 42.02% yield) as a white solid.

LCMS (AM11): rt=0.345 min, (565.0 [M+H]⁺), 100% purity.

¹H NMR (400 MHz, MeOH-d₄) δ 8.62-8.57 (m, 2H), 8.22 (d, J=2.8 Hz, 1H), 7.71-7.66 (m, 2H), 7.54 (d, J=0.8 Hz, 1H), 7.32 (d, J=1.6 Hz, 2H), 6.11 (d, J=4.8 Hz, 1H), 3.75 (s, 2H), 3.40-3.32 (m, 4H), 2.84 (t, J=6.8 Hz, 2H), 2.41 (t, J=6.4 Hz, 2H), 1.97-1.86 (m, 2H) ppm.

### Example 181

### 3-((3-chloro-4-(trifluoromethoxy)benzyl)amino)-N-(3-((7-chloro-6-(4H-1,2,4-triazol-4-yl)-1H-indazol-4-yl)amino)propyl)propanamide

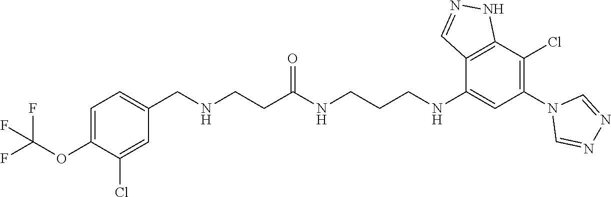 [SMILES: O=C(CCNCc1ccc(OC(F)(F)F)c(Cl)c1)NCCCNc1cc(-n2cnnc2)c(Cl)c2[nH]ncc12]

To a solution of compound 2.322 (55 mg, 72.79 μmol) in DCM (6.8 mL) was added TFA (2.16 g, 18.91 mmol). The mixture was stirred at 20° C. for 2 h. The mixture was concentrated in vacuo to give a residue, which was purified (PM354) to afford EXAMPLE 181 (32.08 mg, 53.79 μmol, 73.89% yield) as a colourless gum.

LCMS (AM14): rt=0.511 min, (571.2 [M+H]⁺), 95.86% purity.

¹H NMR (400 MHz, MeOH-d₄) δ 8.82 (s, 2H), 8.29 (s, 1H), 7.58 (s, 1H), 7.36 (s, 2H), 6.21 (s, 1H), 3.80 (s, 2H), 3.36-3.32 (m, 2H), 3.29 (s, 2H), 2.87 (t, J=6.4 Hz, 2H), 2.42 (t, J=6.8 Hz, 2H), 1.94-1.87 (m, 2H) ppm.

### Example 182

### 7-chloro-N-(2-(4-((3,5-difluoro-4-(trifluoromethoxy)benzyl)amino)butoxy)ethyl)-6-(4H-1,2,4-triazol-4-yl)-1H-indazol-4-amine

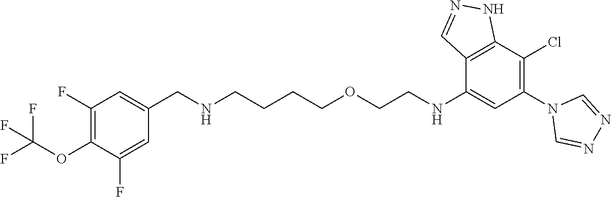 [SMILES: Fc1cc(CNCCCCOCCNc2cc(-n3cnnc3)c(Cl)c3[nH]ncc23)cc(F)c1OC(F)(F)F]

To a solution of compound 2.376 (35 mg, 47.03 μmol) in DCM (6 mL) was added TFA (1.05 mL, 14.18 mmol). The mixture was stirred at 20° C. for 2 h. The mixture was concentrated in vacuo to give a residue, which was purified (PM257) to afford EXAMPLE 182 (16.42 mg, 29.33 μmol, 62.35% yield) as a white solid.

LCMS (AM12): rt=0.436 min, (560.1 [M+H]⁺), 100% purity

¹H NMR (400 MHz, MeOH-d₄) δ 8.83 (s, 2H), 8.30 (s, 1H), 7.16 (d, J=8.4 Hz, 2H), 6.30 (s, 2H), 3.73-3.68 (m, 4H), 3.52 (t, J=5.6 Hz, 2H), 3.47 (t, J=5.2 Hz, 2H), 2.56 (t, J=6.8 Hz, 2H), 1.63-1.55 (m, 4H) ppm

### Example 185

### 3-((3-chloro-4-(trifluoromethoxy)benzyl)amino)-N-(3-((6-(pyridin-4-yl)-1H-pyrazolo[3,4-b]pyridin-4-yl)amino)propyl)propanamide

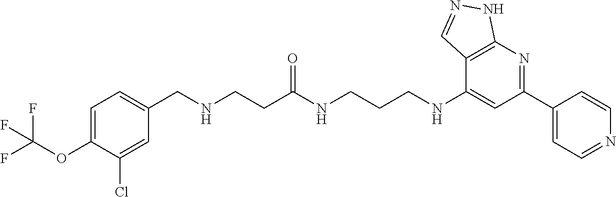 [SMILES: O=C(CCNCc1ccc(OC(F)(F)F)c(Cl)c1)NCCCNc1cc(-c2ccncc2)nc2[nH]ncc12]

To a mixture of compound 2.324 (180 mg, 277.75 μmol) in DCM (6 mL) was added TFA (0.6 mL, 8.1 mmol) at 20° C. The mixture was stirred at 2° C. for 1 h. The mixture was concentrated in vacuo to give a residue, which was purified (PM355) to afford EXAMPLE 185 (14.47 mg, 25.87 μmol, 9.31% yield) as a white solid.

LCMS (AM11): rt=0.314 min, (547.9 [M+H]⁺), 97.95% purity.

¹H NMR (400 MHz, MeOH-d₄) δ 8.64 (dd, J=6.4 Hz, 1.6 Hz, 2H), 8.17 (s, 1H), 8.04 (dd, J=4.8 Hz, 1.6 Hz, 2H), 7.55 (s, 1H), 7.33 (s, 2H), 6.77 (s, 1H), 3.75 (s, 2H), 3.53 (t, J=6.8 Hz, 2H), 3.39 (t, J=6.8 Hz, 2H), 2.85 (t, J=6.8 Hz, 2H), 2.44 (t, J=6.8 Hz, 2H), 2.03-1.92 (m, 2H) ppm.

### Example 186

### 5-chloro-N-(2-(4-((3,5-difluoro-4-(trifluoromethoxy)benzyl)amino)butoxy)ethyl)-6-(4H-1,2,4-triazol-4-yl)-1H-indazol-4-amine

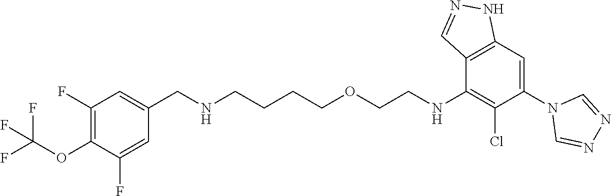 [SMILES: Fc1cc(CNCCCCOCCNc2c(Cl)c(-n3cnnc3)cc3[nH]ncc23)cc(F)c1OC(F)(F)F]

To a solution of compound 2.396 (74 mg, 99.44 μmol) in DCM (5 mL) was added TFA (1 mL, 13.51 mmol). The mixture was stirred at 20° C. for 2 hours. The mixture was concentrated in vacuo to give a residue, which was purified (PM193) to afford EXAMPLE 186 (38.63 mg, 68.03 μmol, 68.41% yield) as a colourless gum.

LCMS (AM14): rt=0.586 min, (560.2, [M+H]⁺), 98.65% purity

¹H NMR (400 MHz, CD₃OD) δ 8.76 (s, 2H), 8.37 (d, J=0.8 Hz, 1H), 7.18 (d, J=8.8 Hz, 2H), 7.00 (d, J=0.8 Hz, 1H), 3.91 (t, J=5.2 Hz, 2H), 3.75 (t, J=6.8 Hz, 4H), 3.54 (t, J=5.6 Hz, 2H), 2.58 (t, J=7.2 Hz, 2H), 1.65-1.60 (m, 4H) ppm

### Example 189

### N-(3-((6-(1,2,4-oxadiazol-3-yl)-1H-indazol-4-yl)amino)propyl)-3-((3-chloro-4-(trifluoromethoxy)benzyl)amino)propanamide

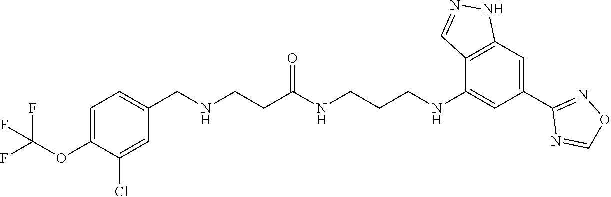 [SMILES: O=C(CCNCc1ccc(OC(F)(F)F)c(Cl)c1)NCCCNc1cc(-c2ncon2)cc2[nH]ncc12]

To a solution of compound 2.038 (50 mg, 68.37 μmol, 87.25% purity) in DCM (2 mL) was added TFA (537.47 mg, 4.71 mmol, 349.00 μL). The mixture was stirred at 20° C. for 1 h. The mixture was concentrated in vacuo to give a residue, which was adjusted to pH=8 with DIPEA and then purified (PM356) to afford EXAMPLE 189 (11.65 mg, 21.58 μmol, 31.56% yield) as a yellow oil.

LCMS (AM3): rt=0.792 min, (538.1 [M+H]⁺), 99.62% purity.

¹H NMR (400 MHz, MeOH-d₄) δ 9.24 (s, 1H), 8.19 (s, 1H), 7.53 (d, J=2.0 Hz, 2H), 7.31 (d, J=2.0 Hz, 2H), 6.83 (s, 1H), 3.76 (s, 2H), 3.39-3.36 (m, 4H), 2.86 (t, J=6.8 Hz, 2H), 2.44 (t, J=6.8 Hz, 2H), 1.95 (t, J=6.8 Hz, 2H) ppm.

¹⁹F NMR (400 MHz, MeOH-d₄) δ −59.624 ppm.

### Example 190

### N4-(2-(4-((3,5-difluoro-4-(trifluoromethoxy)benzyl)amino)butoxy)ethyl)-N3-methyl-6-(4H-1,2,4-triazol-4-yl)-1H-indazole-3,4-diamine

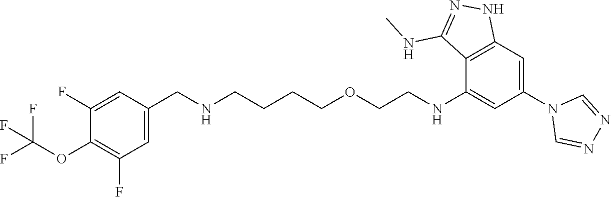 [SMILES: CNc1n[nH]c2cc(-n3cnnc3)cc(NCCOCCCCNCc3cc(F)c(OC(F)(F)F)c(F)c3)c12]

To a solution of compound 2.499 (70 mg, 83.45 μmol) in DCM (5 mL) was added TFA (1 mL, 13.51 mmol). The mixture was stirred at 20° C. for 1 h. The mixture was basified to pH=8 by saturated NaHCO₃ aqueous solution and then concentrated in vacuo at to give a residue, which was purified (PM357) to afford EXAMPLE 190 (75 mg, 75.50 μmol, 39.02% yield) as a brown gum.

LCMS (AM11): rt=0.368 min, (555.3, [M+H]⁺), 99.70% purity.

¹H NMR (400 MHz, MeOH-d₄) δ 8.94 (s, 2H), 7.13 (d, J=8.8 Hz, 2H), 6.71 (d, J=1.6 Hz, 1H), 6.21 (d, J=1.6 Hz, 1H), 3.74 (t, J=4.2 Hz, 2H), 3.70 (s, 2H), 3.55 (t, J=5.6 Hz, 2H), 3.42 (t, J=4.2 Hz, 2H), 2.91 (s, 3H), 2.57 (t, J=9.8 Hz, 2H), 1.65-1.60 (m, 4H) ppm.

### Example 191

### 3-((3-chloro-4-(trifluoromethoxy)benzyl)amino)-N-(3-((3-(methylamino)-6-(4H-1,2,4-triazol-4-yl)-1H-indazol-4-yl)amino)propyl)propanamide

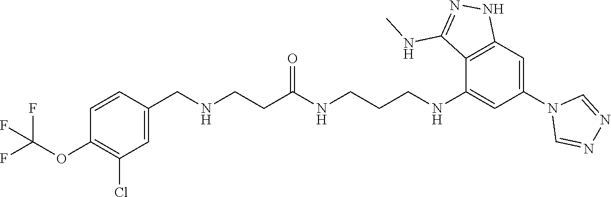 [SMILES: CNc1n[nH]c2cc(-n3cnnc3)cc(NCCCNC(=O)CCNCc3ccc(OC(F)(F)F)c(Cl)c3)c12]

To a solution of compound 2.500 (70 mg, 82.32 μmol) in DCM (5 mL) was added TFA (1 mL, 82.32 μmol). The mixture was stirred at 20° C. for 1 h. The mixture was basified to pH=8 by NaHCO₃ (1 mL) aqueous solution. The resulting mixture was concentrated in vacuo to give a residue, which was purified (PM177) to afford EXAMPLE 191 (13.38 mg, 22.90 μmol, 27.82% yield) as a brown gum.

LCMS (AM11): rt=0.340 min, (566.1[M+H]⁺), 96.87% purity.

¹H NMR (400 MHz, MeOH-d₄) δ 8.95 (s, 2H), 7.55 (s, 1H), 7.33 (s, 2H), 6.69 (d, J=1.6 Hz, 1H), 6.15 (d, J=1.2 Hz, 1H), 3.75 (s, 2H), 3.37-3.30 (m, 4H), 2.93 (s, 3H), 2.84 (t, J=7.2 Hz, 2H), 2.42 (t, J=7.6 Hz, 2H), 1.90 (t, J=6.8 Hz, 2H) ppm.

### Example 192

### (S)-(3-(((5-(2-((6-(4H-1,2,4-triazol-4-yl)-1H-indazol-4-yl)amino)ethoxy)pentan-2-yl)amino)methyl)-5-(trifluoromethoxy)phenyl)methanol

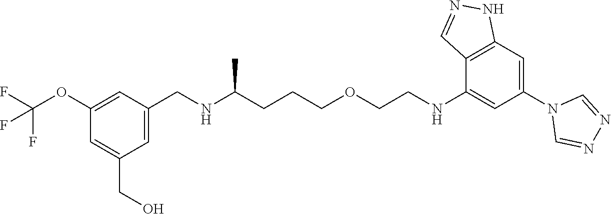 [SMILES: C[C@@H](CCCOCCNc1cc(-n2cnnc2)cc2[nH]ncc12)NCc1cc(CO)cc(OC(F)(F)F)c1]

To a solution of compound 2.432 (200 mg, 455.81 μmol, HCl salt) in MeOH (4 mL) was added 3-(Hydroxymethyl)-5-(trifluoromethoxy)benzaldehyde (Ref: WO2022185041, 200.69 mg, 911.61 μmol) and DABCO (255.64 mg, 2.28 mmol). The mixture was stirred at 20° C. for 1 h, then NaBH₃CN (57.29 mg, 911.61 μmol) was added. The mixture was stirred at 20° C. for 12 h. The mixture was filtered and the filtrate was purified (PM250) to afford EXAMPLE 192 (84.37 mg, 157.06 μmol, 34.46% yield) as a yellow solid.

LCMS (AM11): rt=0.304 min, (534.3, [M+H]⁺), 99.32% purity

¹H NMR (400 MHz, MeOH-d₄) δ 8.98 (s, 2H), 8.20 (s, 1H), 7.26 (s, 1H), 7.15 (br d, J=5.2 Hz, 2H), 6.92 (s, 1H), 6.35 (d, J=1.2 Hz, 1H), 4.61 (s, 2H), 3.78-3.68 (m, 4H), 3.52 (q, J=5.6 Hz, 4H), 2.69-2.59 (m, 1H), 1.65-1.53 (m, 3H), 1.45-1.31 (m, 1H), 1.05 (d, J=6.4 Hz, 3H) ppm

### Example 193

### (S)-5-(4-((2-((4-((3-(hydroxymethyl)-5-(trifluoromethoxy)benzyl)amino)pentyl)oxy)ethyl)amino)-1H-indazol-6-yl)pyridazin-3-ol

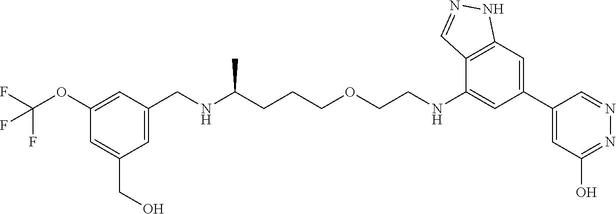 [SMILES: C[C@@H](CCCOCCNc1cc(-c2cnnc(O)c2)cc2[nH]ncc12)NCc1cc(CO)cc(OC(F)(F)F)c1]

To a solution of compound 2.463 (100 mg, 214.68 μmol, HCl salt) in MeOH (4 mL) was added 3-(Hydroxymethyl)-5-(trifluoromethoxy)benzaldehyde (Ref: WO2022185041, 94.52 mg, 429.36 μmol) and DABCO (168.57 mg, 1.50 mmol). The mixture was stirred at 20° C. for 1 h, then NaBH₃CN (26.98 mg, 429.36 μmol) was added. The mixture was stirred at 20° C. for 12 h. The mixture was filtered and the filtrate was directly purified (PM370) and further purified (PM358) to afford EXAMPLE 193 (31.11 mg, 38.81 μmol, 18.08% yield, TFA salt) as a yellow solid.

LCMS (AM12): rt=0.406 min, (561.1, [M+H]⁺), 98.38% purity.

¹H NMR (400 MHz, MeOH-d₄) δ 8.33 (d, J=2.0 Hz, 1H), 8.16 (s, 1H), 7.37 (s, 1H), 7.35 (s, 1H), 7.25 (s, 1H), 7.13 (d, J=2.0 Hz, 1H), 7.10 (s, 1H), 6.46 (d, J=0.8 Hz, 1H), 4.65 (s, 2H), 4.16-4.07 (m, 2H), 3.81-3.75 (m, 2H), 3.62-3.55 (m, 4H), 3.37-3.34 (m, 1H), 2.01-1.90 (m, 1H), 1.83-1.72 (m, 1H), 1.69-1.62 (m, 2H), 1.34 (d, J=6.4 Hz, 3H) ppm

The following examples in Table 30 were made with non-critical changes or substitutions to the exemplified procedure for Example 193, that would be understood by one skilled in the art, using Intermediate 2.463

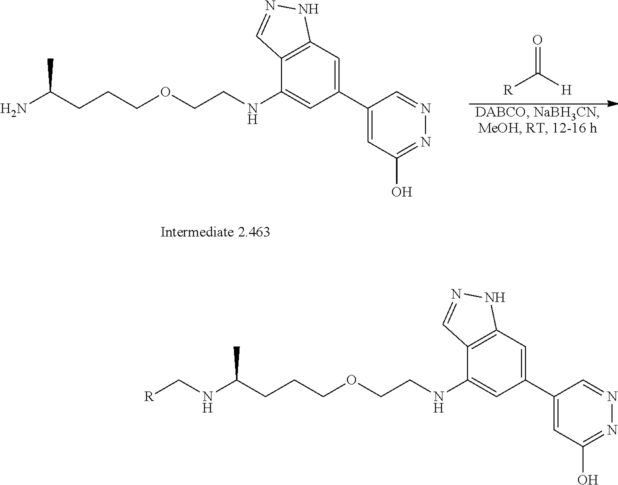

TABLE 30

| | | Aldehyde | |
|---|---|---|---|
| Example | Chemical | (Intermediate | |
| No. | IUPAC name | No./CAS) | Analytical |
| Example 194 | (S)-2-(3-(((5- (2-((6-(6- hydroxypyrida- zin-4-yl)-1H- indazol-4- yl)amino)ethoxy) pentan-2- yl)amino)meth- yl)-5- | 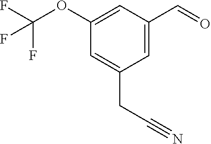 [SMILES: N#CCc1cc(C=O)cc(OC(F)(F)F)c1] | ¹H NMR (400 MHz, DMSO-d₆) δ 13.13- 12.90 (m, 2H), 8.81-8.56 (m, 2H), 8.29 (d, J = 1.6 Hz, 1H), 8.23 (s, 1H), 7.52 (br d, J = 6.4 Hz, 2H), 7.43 (s, 1H), 7.09 (s, 1H), 7.05 (s, 1H), 6.59-6.40 (m,1H), 6.36 (s, 1H), 4.23-4.16 (m, 4H), 3.66 (t, J = 5.6 Hz, 2H), 3.48 (br t, J = 5.2 Hz, 4H), 3.27-3.22 (m, 1H), 1.91-1.79 (m, 1H) 1.67-1.51 (m, 3H), 1.25 (d, J = 6.4 Hz, 3H) ppm |
| | (trifluoromethoxy) | 2-(3-Formyl-5- | LCMS (AM11): rt = 0.327 min, (570.1 |
| | phenyl)aceto- | (trifluoromethoxy) | [M + H]⁺), 97.26% purity |
| | nitrile | phenyl)acetoni- | Purification method PM359 |
| | | trile (Ref: | |
| | | WO2022185041) | |
| Example 195 | (S)-5-(4-((2- ((4-((3-(oxazol- 5-ylmethyl)-5- (trifluoromethoxy) benzyl)ami- no)pentyl)oxy) ethyl)amino)- 1H-indazol-6- yl)pyridazin-3- ol | 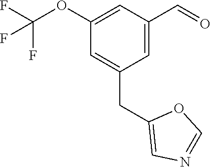 [SMILES: O=Cc1cc(Cc2cnco2)cc(OC(F)(F)F)c1] | ¹H NMR (400 MHz, MeOH-d₄) δ 8.31 (d, J = 2.0 Hz, 1H), 8.18 (s, 1H), 8.09 (s, 1H), 7.18 (s, 1H), 7.13 (br d, J = 2.0 Hz, 2H), 7.08 (s, 1H), 7.04 (s, 1H), 6.88 (s, 1H), 6.43 (s, 1H), 4.05 (s, 2H), 3.75-3.63 (m, 4H), 3.52 (dt, J = 14.4, 5.6 Hz, 4H), 2.64-2.54 (m, 1H), 1.65-1.52 (m, 3H), 1.41-1.30 (m, 1H), 1.03 (d, J = 6.0 Hz, 3H) ppm LCMS (AM11): rt = 0.337 min, (612.4 [M + H]⁺), 99.76% purity Purification method PM360 |
| | | 3-(Oxazol-5- | |
| | | ylmethyl)-5- | |
| | | (trifluoromethoxy) | |
| | | benzaldehyde | |
| | | (Ref: | |
| | | WO2022185041) | |

### Example 196

### (S)-3-(((5-(2-((6-(isoxazol-4-yl)-1H-indazol-4-yl)amino)ethoxy)pentan-2-yl)amino)methyl)-5-(trifluoromethoxy)benzamide

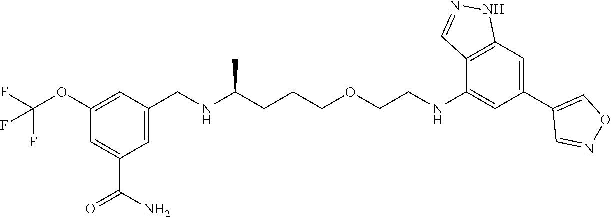 [SMILES: C[C@@H](CCCOCCNc1cc(-c2cnoc2)cc2[nH]ncc12)NCc1cc(OC(F)(F)F)cc(C(N)=O)c1]

To a solution of compound 2.468 (60 mg, 89.36 μmol, TFA salt) and 3-Formyl-5-(trifluoromethoxy)benzamide (Ref: WO2022185041, 27.08 mg, 116.16 μmol) in MeOH (4 mL) was added DABCO (50.12 mg, 446.78 μmol). The mixture was stirred at 20° C. for 1 h, then NaBH₃CN (11.23 mg, 178.71 μmol) was added. The mixture was stirred at 20° C. for 1 h. The mixture was acidified to pH=6 by 10% TFA aqueous solution and then directly purified (PM361) to afford EXAMPLE 196 (21.63 mg, 27.41 μmol, 30.67% yield, 2×TFA salt) as a brown gum.

LCMS (AM15): rt=0.549 min, (547.2, [M+H]⁺), 98.15% purity.

¹H NMR (400 MHz, DMSO-d₆) δ 12.83 (br s, 1H), 9.42 (s, 1H), 9.15 (s, 1H), 8.81-8.72 (m, 1H), 8.70-8.61 (m, 1H), 8.23-8.12 (m, 2H), 8.09 (s, 1H), 7.91 (s, 1H), 7.72-7.63 (m, 2H), 6.97 (s, 1H), 6.36 (s, 1H), 4.31-4.16 (m, 2H), 3.67 (t, J=6.0 Hz, 2H), 3.30-3.19 (m, 2H), 2.59-2.52 (m, 2H), 2.48-2.42 (m, 1H), 1.88-1.77 (m, 1H), 1.70-1.60 (m, 1H), 1.59-1.47 (m, 2H), 1.25 (d, J=6.8 Hz, 3H) ppm

### Example 197

### 4-((2-(4-((3,5-difluoro-4-(trifluoromethoxy)benzyl)amino)butoxy)ethyl)amino)-1H-indazole-6-carboxylic acid

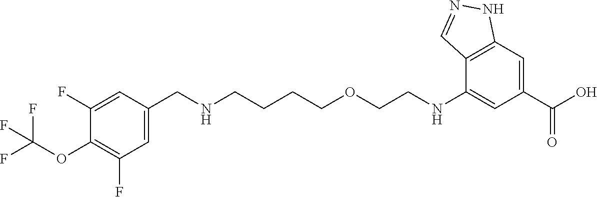 [SMILES: O=C(O)c1cc(NCCOCCCCNCc2cc(F)c(OC(F)(F)F)c(F)c2)c2cn[nH]c2c1]

To a solution of compound 2.512 (80 mg, 132.77 μmol) in DCM (4 mL) was added TFA (1.23 g, 10.80 mmol). The mixture was stirred at 20° C. for 1 h. The mixture was concentrated in vacuo to give a residue, which was purified (PM362) to afford EXAMPLE 197 (28.89 mg, 46.86 μmol, 35.30% yield, TFA salt) as a yellow solid.

LCMS (AM11): rt=0.373 min, (503.5 [M+H]⁺), 100.00% purity.

¹H NMR (400 MHz, MeOH-d₄) δ 8.17 (s, 1H), 7.52 (s, 1H), 7.29 (s, 1H), 7.27 (s, 1H), 6.85 (s, 1H), 4.10 (s, 2H), 3.75 (t, J=5.6 Hz, 2H), 3.59 (t, J=5.6 Hz, 2H), 3.53 (t, J=5.2 Hz, 2H), 3.10 (t, J=8.8 Hz, 2H), 1.83 (t, J=7.60 Hz, 2H), 1.75-1.68 (m, 2H) ppm.

### Example 199

### 5-(4-(2-((3-((3-(2-hydroxyethoxy)-5-(trifluoromethoxy)benzyl)amino)cyclobutyl)methoxy)ethoxy)-1H-indazol-6-yl)pyridazin-3-ol

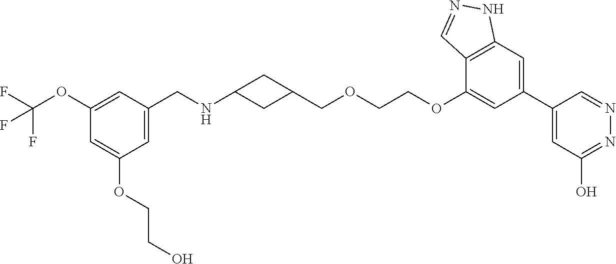 [SMILES: OCCOc1cc(CNC2CC(COCCOc3cc(-c4cnnc(O)c4)cc4[nH]ncc34)C2)cc(OC(F)(F)F)c1]

To a solution of compound 2.411 (80 mg, 186.78 μmol, HCl salt) in MeOH (1 mL) was added DABCO (104.76 mg, 933.90 μmol) and 3-(2-hydroxyethoxy)-5-(trifluoromethoxy)benzaldehyde (Ref: WO2022185041, 46.73 mg, 186.78 μmol). The mixture was stirred at 20° C. for 12 h, then NaBH₃CN (23.48 mg, 373.56 μmol) was added and the mixture was stirred at 20° C. for 0.5 h. The reaction was acidified to pH=5 by 20% TFA aqueous solution and filtered. The filtrate was purified (PM358) to afford compound EXAMPLE 199 (45.09 mg, 64.09 μmol, 34.31% yield, TFA salt) as a yellow solid.

LCMS (AM11): rt=0.349 min, (590.5, [M+H]⁺), 100.00% purity.

¹H NMR (400 MHz, MeOH-d₄) δ 8.41-8.35 (m, 1H), 8.11 (s, 1H), 8.51 (d, J=4.0 Hz, 1H), 7.22-7.17 (m, 1H), 7.03 (d, J=8.0 Hz, 1H), 6.97 (d, J=3.6 Hz, 1H), 6.92 (s, 1H), 6.86 (d, J=13.2 Hz, 1H), 4.44 (d, J=2.0 Hz, 2H), 4.11-4.02 (m, 4H), 3.97 (d, J=2.4 Hz, 2H), 3.92-3.85 (m, 2.5H), 3.75-3.70 (m, 0.5H), 3.66 (dd, J=25.8 Hz, 5.6 Hz, 2H), 3.72-3.61 (m, 0.5H), 2.57-2.49 (m, 0.5H), 2.48-2.41 (m, 1H), 2.40-2.71 (m, 2H), 2.15-2.02 (m, 1H) ppm.

The following examples in Table 31 were made with non-critical changes or substitutions to the exemplified procedure for Example 199, that would be understood by one skilled in the art, using Intermediate 2.411

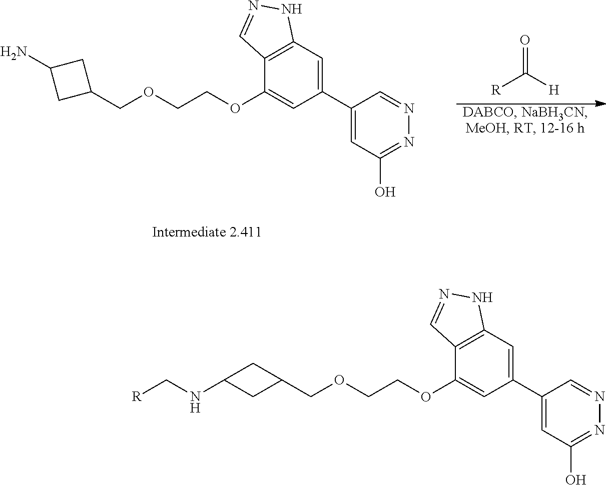

TABLE 31

| | | Aldehyde | |
|---|---|---|---|
| Example | Chemical | (Intermediate | |
| No. | IUPAC name | No./CAS) | Analytical |
| Example 200 | 5-(4-(2-((3- ((3- (hydroxymeth- yl)-5- (trifluorometh- oxy)benzyl)a- mino)cyclobu- tyl)methoxy)eth- | 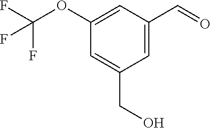 [SMILES: O=Cc1cc(CO)cc(OC(F)(F)F)c1] | ¹H NMR (MeOH-d₄, 400 MHz) δ 8.37 (d, J = 2.0 Hz, 1H), 8.11 (s, 1H), 7.50 (s, 1H), 7.40 (s, 1H), 7.34 (s, 1H), 7.30 (s, 1H), 7.22-7.15 (m, 1H), 6.85 (s, 1H), 4.65 (s, 2H), 4.43 (s, 2H), 4.12 (s, 2H), 3.97 (s, 2H), 3.76-3.59 (m, 3H), 2.72- 2.49 (m, 1H), 2.48-2.30 (m, 2.5 H), 2.13- 2.02 (m, 1.5H) ppm |
| | oxy)-1H- | 3- | LCMS (AM11): rt = 0.343 min, (560.4 |
| | indazol-6- | (Hydroxymethyl)- | [M + H]⁺), 99.10% purity |
| | yl)pyridazin- | 5- | Purification method PM264 |
| | 3-ol | (trifluoromethoxy) | |
| | | benzaldehyde | |
| | | (Ref: | |
| | | WO2022185041) | |

### Example 201

### 3-(4-((2-(4-((3,5-difluoro-4-(trifluoromethoxy)benzyl)amino)butoxy)ethyl)amino)-1H-indazol-6-yl)propanoic acid

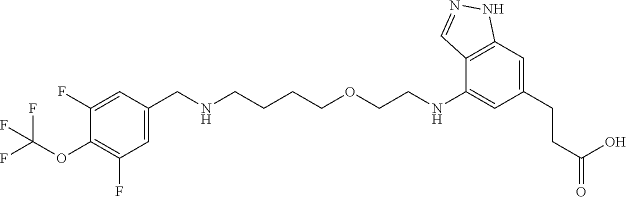 [SMILES: O=C(O)CCc1cc(NCCOCCCCNCc2cc(F)c(OC(F)(F)F)c(F)c2)c2cn[nH]c2c1]

To a solution of compound 2.515 (90 mg, 116.76 μmol) in DCM (9 mL) was added TFA (3 mL, 40.52 mmol), then the mixture was stirred at 20° C. for 1 h. The reaction was concentrated in vacuo to give a residue, which was purified (PM362) to afford EXAMPLE 201 (46.23 mg, 71.73 μmol, 61.43% yield, TFA salt) as a brown gum.

LCMS (AM11): rt=0.369 min, (531.6, [M+H]⁺), 100% purity

¹H NMR (400 MHz, DMSO-d₆) δ 12.52 (br s, 1H), 8.86 (s, 2H), 8.06 (s, 1H), 7.54 (d, J=8.8 Hz, 2H), 6.50 (s, 1H), 5.94 (s, 1H), 4.17 (t, J=5.2 Hz, 2H), 3.62 (s, 2H), 3.46 (s, 2H), 3.36-3.33 (m, 2H), 2.96 (br s, 2H), 2.81 (t, J=7.2 Hz, 2H), 2.56-2.53 (m, 2H), 1.70-1.57 (m, 4H) ppm

### Example 202

### 2-(3-(((4-(2-((1H-indazol-4-yl)amino)ethoxy)butyl)amino)methyl)-5-(trifluoromethoxy)phenoxy)ethan-1-ol

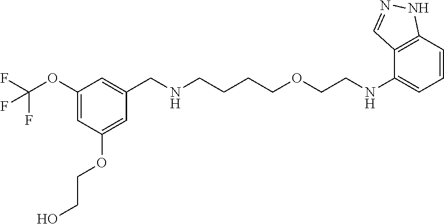 [SMILES: OCCOc1cc(CNCCCCOCCNc2cccc3[nH]ncc23)cc(OC(F)(F)F)c1]

To a solution of 3-(2-hydroxyethoxy)-5-(trifluoromethoxy)benzaldehyde (Ref: WO2022185041, 48.96 mg, 195.69 μmol) in MeOH (1.5 mL) was added compound 2.576 (70 mg, 195.69 μmol) and DABCO (109.75 mg, 978.46 μmol). The mixture was stirred at 20° C. for 3 h, then NaBH₃CN (43.04 mg, 684.92 μmol) was added. The resulting mixture was at for 20° C. for 1 h. The reaction mixture was acidified to pH=6 by 20% TFA aqueous solution and filtered. The filtrate was directly purified (PM363), then further purified (PM364) to afford EXAMPLE 202 (1.8 mg, 3.72 μmol, 1.90% yield, 99.60% purity) as a yellow gum

LCMS (AM11): rt=0.348 min, (483.3, [M+H]⁺), 100% purity.

¹H NMR (400 MHz, DMSO-d₆) δ 12.66 (s, 1H), 8.14 (s, 1H), 7.05 (t, J=8.0 Hz, 1H), 6.94 (s, 1H), 6.88 (s, 1H), 6.75 (s, 1H), 6.65 (d, J=8.4 Hz, 1H), 6.14 (t, J=5.2 Hz, 1H), 6.03 (d, J=7.2 Hz, 1H), 4.86 (t, J=5.2 Hz, 1H), 4.00 (t, J=4.8 Hz, 2H), 3.72-3.68 (m, 2H), 3.67 (s, 2H), 3.59 (t, J=5.6 Hz, 2H), 3.42 (t, J=6.4 Hz, 4H), 2.48-2.46 (m, 2H), 1.58-1.52 (m, 2H), 1.48-1.43 (m, 2H).

### Example 203

### (3-(((3-((2-((6-(1,2,3-thiadiazol-5-yl)-1H-indazol-4-yl)amino)ethoxy)methyl)cyclobutyl)amino)methyl)-5-(trifluoromethoxy)phenyl)methanol

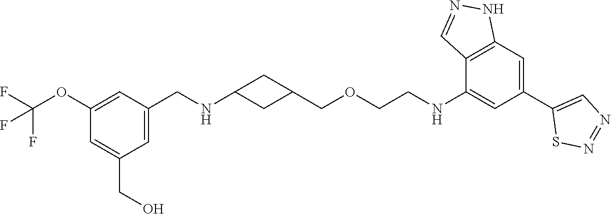 [SMILES: OCc1cc(CNC2CC(COCCNc3cc(-c4cnns4)cc4[nH]ncc34)C2)cc(OC(F)(F)F)c1]

To a solution of compound 2.529 (60 mg, 135.93 μmol) and 3-(hydroxymethyl)-5-(trifluoromethoxy)benzaldehyde (Ref: WO2022185041, 32.92 mg, 149.52 μmol) in MeOH (2 mL) was added DABCO (45.74 mg, 407.79 μmol) in one portion. The mixture was stirred at 25° C. for 12 h, then NaBH₃CN (12.81 mg, 203.90 μmol) was added and the mixture was stirred at 25° C. for 1 h. The mixture was acidified to pH=4 by 20% TFA aqueous solution and filtered. The filtrate was directly purified (PM359) to afford EXAMPLE 203 (13.36 mg, 20.06 μmol, 14.75% yield, TFA salt) as a yellow solid.

LCMS (AM11): rt=0.368 min, (549.3 [M+H]⁺), 99.47% purity

¹H NMR (400 MHz, MeOH-d₄) δ 9.16-9.08 (m, 1H), 8.17 (s, 1H), 7.40 (s, 1H), 7.35 (s, 1H), 7.28 (s, 1H), 7.16 (s, 1H), 6.52-6.40 (m, 1H), 4.67-4.63 (m, 2H), 4.13-3.99 (m, 2H), 3.83-3.76 (m, 2H), 3.68 (t, J=7.6 Hz, 1H), 3.60-3.51 (m, 4H), 2.65-2.28 (m, 3.5H), 2.06-1.98 (m, 1.5H) ppm.

The following examples in Table 32 were made with non-critical changes or substitutions to the exemplified procedure for Example 203, that would be understood by one skilled in the art, using Intermediate 2.529

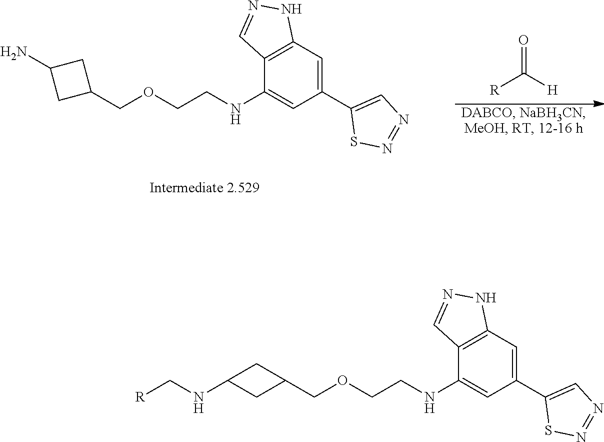

TABLE 32

| | | Aldehyde | |
|---|---|---|---|
| Example | Chemical | (Intermediate | |
| No. | IUPAC name | No./CAS) | Analytical |
| Example 205 | 2-(3-(((3-((2- ((6-(1,2,3- thiadiazol-5- yl)-1H- indazol-4- yl)amino)ethoxy) methyl)cy- clobutyl)amino) | 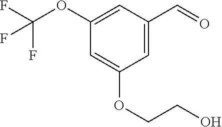 [SMILES: O=Cc1cc(OCCO)cc(OC(F)(F)F)c1] | ¹H NMR (400 MHz, DMSO-d₆) δ 13.19- 12.86 (m, 1H), 9.41 (s, 1H), 8.25 (s, 1H), 7.11 (s, 1H), 6.92 (s, 1H), 6.85 (s, 1H), 6.74 (s, 1H), 6.62-6.55 (m, 1H), 6.39 (s, 1H), 4.99-4.77 (m, 1H), 3.99 (t, J = 4.8 Hz, 2H), 3.70 (t, J = 4.8 Hz, 2H), 3.66- 3.60 (m, 2H), 3.59-3.52 (m, 2H), 3.47- 3.43 (m, 2H), 3.39 (d, J = 6.0 Hz, 1H), |
| | methyl)-5- | 3-(2- | 3.25 (s, 1H), 3.05-2.90 (m, 1H), 2.30- |
| | (trifluorometh- | Hydroxyethoxy)- | 2.05 (m, 0.5H), 2.18-2.07 (m, 2H), 1.88- |
| | oxy)phenoxy) | 5- | 1.76 (m, 1H), 1.45-1.35 (m, 1.5H) |
| | ethan-1-ol | (trifluoromethoxy) | ppm. |
| | | benzaldehyde | LCMS (AM11): rt = 0.365 min, (579.2, |
| | | (Ref: | [M + H]⁺), 99.58% purity. |
| | | WO2022185041) | Purification method PM365 |

### Example 204

### 2-(4-((2-(4-((3-(hydroxymethyl)-5-(trifluoromethoxy)benzyl)amino)butoxy)ethyl)amino)-1H-indazol-6-yl)acetonitrile

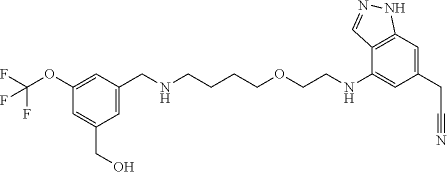 [SMILES: N#CCc1cc(NCCOCCCCNCc2cc(CO)cc(OC(F)(F)F)c2)c2cn[nH]c2c1]

To a solution of compound 2.454 (100 mg, 194.02 μmol, TFA salt) in MeOH (2 mL) was added 3-(hydroxymethyl)-5-(trifluoromethoxy)benzaldehyde (Ref: WO2022185041, 46.98 mg, 213.42 μmol) and DABCO (76.17 mg, 679.08 μmol). The mixture was stirred at 20° C. for 1 h, then NaBH₃CN (24.39 mg, 388.04 μmol) was added and the mixture was stirred at 20° C. for 0.5 h. The reaction mixture was acidified to pH=5 by 20% TFA aqueous solution and filtered. The filtrate was directly purified (PM358) to afford EXAMPLE 204 (13.02 mg, 20.79 μmol, 10.72% yield, TFA salt) as a brown gum.

LCMS (AM11): rt=0.321 min, (492.1 [M+H]⁺), 96.69% purity.

¹H NMR (400 MHz, DMSO-d₆) δ 12.74 (br. s, 1H), 8.92 (s, 2H), 8.15 (s, 1H), 7.45 (s, 1H), 7.37 (s, 1H), 7.33 (s, 1H), 6.67 (s, 1H), 6.00 (s, 1H), 4.57 (s, 2H), 4.16 (t, J=6.0 Hz, 2H), 3.97 (s, 2H), 3.62 (t, J=6.0 Hz, 2H), 3.46 (t, J=6.4 Hz, 2H), 3.36 (t, J=5.6 Hz, 2H), 3.00-2.90 (s, 2H), 1.73-1.64 (m, 2H), 1.62-1.53 (m, 2H) ppm.

The following examples in Table 33 were made with non-critical changes or substitutions to the exemplified procedure for Example 204, that would be understood by one skilled in the art, using 3-(hydroxymethyl)-5-(trifluoromethoxy)benzaldehyde (Ref: WO2022185041)

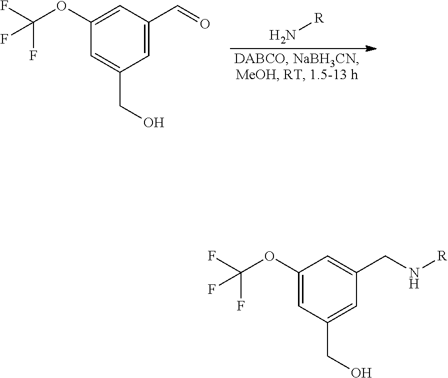

TABLE 33

| Example | Chemical IUPAC | | |
|---|---|---|---|
| No. | name | H₂NR | Analytical |
| Example 206 | 2-(4-((2-(4-((3- (hydroxymethyl)- 5- (trifluoromethoxy) benzyl)amino)butoxy) ethyl)amino)- 1H-indazol-6-yl)- 2- methylpropanenitrile | 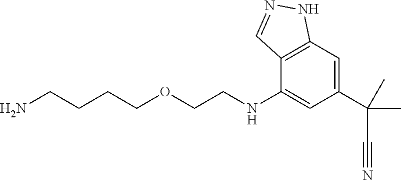 | ¹H NMR (400 MHz, DMSO-d₆) δ 13.00-12.64 (m, 1H), 8.87-8.71 (m, 2H), 8.15 (s, 1H), 7.46 (s, 1H), 7.37 (s, 1H), 7.34 (s, 1H), 6.77 (s, 1H), 6.16 (d, J = 1.2 Hz, 1H), 4.57 (s, 2H), 4.21-4.11 (m, 2H), 3.64 (t, J = 5.6 Hz, 2H), 3.47 (t, J = 6.0 Hz, 2H), 3.41 (t, J = 6.0 Hz, 2H), 3.00- 2.90 (m, 2H), 1.70 (s, 6H), 1.69- 1.63 (m, 2H), 1.62-1.54 (m, 2 H) ppm. |
| | | Compound 2.537 | LCMS (AM22): rt = 0.795 min, |
| | | | (520.4, [M + H]⁺), 99.54% purity. |
| | | | Purification method 1) PM366 |
| | | | then 2) PM367 |
| Example 208 | (R)-(3-(((4-(2-((6- (1,2,3-thiadiazol-5- yl)-1H-indazol-4- yl)amino)propoxy) butyl)amino)methyl)- 5- (trifluoromethoxy)phe- nyl)methanol | 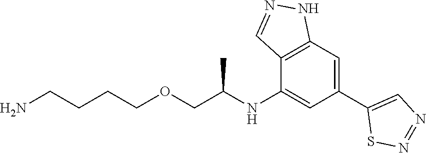 | ¹H NMR (400 MHz, MeOH-d₄) δ 9.11 (s, 1H), 8.22 (s, 1H), 7.24 (s, 1H), 7.17 (s, 1H), 7.12 (s, 2H), 6.49 (s, 1H), 4.62 (s, 2H), 4.01-3.94 (m, 1H), 3.69 (s, 2H), 3.62-3.58 (m, 1H), 3.57- 3.50 (m, 3H), 2.56 (t, J = 7.2 Hz, 2H), 1.61-1.58 (m, 4H), 1.33 (d, J = 6.4 Hz, 3H) ppm. LCMS (AM12): rt = 0.437 min, |
| | | Compound 2.548 | (551.4, [M + H]⁺), 98.27% purity. |
| | | | Purification method 1) PM209 |
| | | | then 2) PM368 |

### Example 207

### (S)-(3-(((5-(2-((6-(1,2,3-thiadiazol-5-yl)-1H-indazol-4-yl)amino)ethoxy)pentan-2-yl)amino)methyl)-5-(trifluoromethoxy)phenyl)methanol

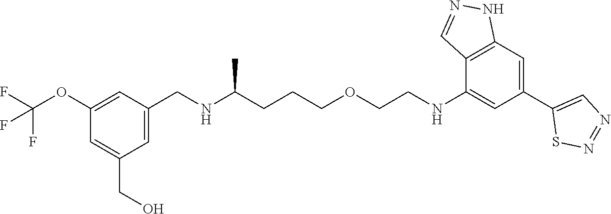 [SMILES: C[C@@H](CCCOCCNc1cc(-c2cnns2)cc2[nH]ncc12)NCc1cc(CO)cc(OC(F)(F)F)c1]

To a solution of compound 2.531 (60 mg, 143.07 μmol, HCl salt) in MeOH (3 mL) was added 3-(hydroxymethyl)-5-(trifluoromethoxy)benzaldehyde (Ref: WO2022185041, 37.80 mg, 171.69 μmol) and DABCO (80.24 mg, 715.36 μmol). The mixture was stirred at 20° C. for 12 h, then NaBH₃CN (26.97 mg, 429.21 μmol) was added. The mixture was stirred at 20° C. for 1 h. The mixture was filtered and the filtrate was purified (PM369) to afford EXAMPLE 207 (30.79 mg, 55.44 μmol, 38.75% yield) as a yellow solid.

LCMS (AM22): rt=0.438 min, (551.4, [M+H]⁺), 99.14% purity

¹H NMR (400 MHz, MeOH-d₄) δ 9.12 (s, 1H), 8.17 (s, 1H), 7.24 (s, 1H), 7.16 (s, 1H), 7.13 (s, 2H), 6.47 (s, 1H), 4.60 (s, 2H), 3.77-3.65 (m, 4H), 3.53 (q, J=5.6 Hz, 4H), 2.68-2.59 (m, 1H), 1.67-1.54 (m, 3H), 1.43-1.33 (m, 1H), 1.05 (d, J=6.4 Hz, 3H) ppm

### Biological Assays

### Assay 1: Biochemical Assay for Inhibitors of CK2α Kinase Activity

The inhibitory activity of putative kinase inhibitors and the potency of selected compounds were determined using ADP-Glo™ assay. The kinase reaction was performed in the presence of excess peptide substrate and ATP at a concentration equivalent to Km. Upon termination of the kinase reaction, remaining ATP was depleted leaving only ADP reaction product, which was converted back to ATP with a coupled luciferin/luciferase reaction. The luminescent output from the coupled reaction was quantified and correlated with the kinase activity.

CK2α (residues 2-329) was produced in Escherichia coli BL21 (DE3) for kinase activity screening. Single colonies of the cells were grown in 6×1 L of 2×TY with 100 μg/mL ampicillin at 37° C. Isopropyl thio-β-D-galactopyranoside (IPTG) was added to a final concentration of 0.4 mM to induce expression when the optical density at 600 nm reached 0.6. The cells were incubated overnight at 25° C. then harvested by centrifugation at 4,000 g for 20 minutes. The cell pellets were suspended in 20 mM Tris, 500 mM NaCl, pH 8.0 and lysed using a high pressure homogenizer. Protease inhibitor cocktail tablets (one tablet per 50 mL extract; Roche Diagnostics) and DNase I were then added. The crude cell extract was then centrifuged at 10,000 g for 45 minutes, the supernatant was filtered with a 0.22 μm filter. The soluble supernatant was applied on a Ni Sepharose Fast Flow6 column at pH 8.0, washed and eluted in 20 mM Tris pH 8.0, 500 mM NaCl, 200 mM imidazole. After overnight dialysis into 20 mM Tris, pH 8.0, 500 mM NaCl the N-terminal His6-tag was cleaved overnight by TEV protease and passed through a second metal affinity column to remove uncleaved protein and the protease. The cleaved protein was further purified on a Sepharose Q HP anion-exchange column and the main peak fraction from this column was further purified by gel filtration on a Superdex 75 16/60 HiPrep column equilibrated with Tris 20 mM, pH 8.0, 500 mM NaCl. Pure protein was concentrated to 15 mg/mL and flash frozen in liquid nitrogen.

Final assay conditions comprised 0.2 nM CK2α, 50 μM peptide substrate (RRRADDSDDDDD), 15 μM ATP in 1× reaction buffer (40 mM Tris pH7.5, 200 mM NaCl, 20 mM MgCl₂, 0.1 mg/mL BSA, 1% DMSO). The assay was conducted as follows:
1. Appropriate serial dilutions of test compound were prepared using Echo (Labcyte) and 50 nL of 100× compound in 100% DMSO transferred to the assay plate (white opaque OptiPlate-384, Perkin-Elmer).
2. Enzyme and peptide substrate were prepared in fresh reaction buffer and added to the assay plate in a total volume of 3 μl and incubated at RT for 15 minutes.
3. 2 μL of ATP solution freshly prepared in reaction buffer was added to start the reaction.
4. After 120 minutes, the reaction was stopped by addition of 5 pl ADP-Glo reagent (Promega V9102) and the plate incubated at RT for a further 60 minutes.
5. 10 μL of Kinase Detection reagent (Promega V9102) was added to assay plate and incubated for a further 30 minutes prior to reading luminescence on an Envision (Perkin-Elmer).

Data was analysed to calculate compound IC₅₀and Kᵢ as follows:
1. All assay plates contained 32 wells designated as 0% inhibition control wells, which were treated with vehicle only (1% DMSO) and 32 wells designated as 100% inhibition control wells, which were treated with a high concentration of non-specific kinase inhibitor in 1% DMSO.
2. Percent inhibition in each test well was calculated using the formula (MEAN_{0%inhibition control wells}−test well reading)/(MEAN_{0% inhibition control wells}−MEAN_{100%inhibition control wells})×100%.
3. IC₅₀ was determined using a standard 4-parameter fit method (Model 205, XL-fit).
4. Percent activity was calculated for each well using: (Test well reading−MEAN_{100%inhibition control wells})/(MEAN_{0% inhibition control wells}−MEAN_{100%inhibition control wells}).
5. Morrison Kᵢ was determined using Morrison Kᵢ equation (XL-fit).

### Assay 2: Biochemical Assay for Inhibitors of CLK2 Kinase Activity

The assay was conducted in the same way as described for CK2α, with final assay conditions comprising 20 nM CLK2 (Carna Biosciences-04-127), 50 μM peptide substrate (KRRRLASLR), 100 μM ATP in 1× reaction buffer (40 mM Tris pH7.5, 200 mM NaCl, 20 mM MgCl2, 0.1 mg/mL BSA, 1% DMSO).

### Assay 3: Cell-Based NanoBRET™ Assay for Inhibitor Binding to Intracellular CK2α

This assay used the NanoBRET™ System (Promega), an energy transfer technique designed to measure molecular proximity in living cells. The assay measured the apparent affinity of test compounds by competitive displacement of a NanoBRET™ tracer reversibly bound to a NanoLucR luciferase CK2α fusion protein in cells. A fixed concentration of tracer was added to cells expressing the desired NanoLucR-CK2α fusion protein to generate a BRET reporter complex. Introduction of competing compounds resulted in a dose-dependent decrease in NanoBRET™ energy transfer, which allowed quantitation of the apparent intracellular affinity of the target protein for the test compound.

The assay was conducted as follows using HCT116 cell line (ATCC CCL-247™) transiently transfected with CSNK2A2-NanoLuc® Fusion Vector (Promega NV1191):
1. Cells were resuspended to 2×10⁵ cells/mL in Opti-MEM (Invitrogen 11058021).
2. DNA complex was prepared in a final volume of 1.4 ml Opti-MEM containing 15 μg DNA and 42 μl FuGENE HD Transfection reagent (Promega E2311).
3. 20 ml cell suspension was combined with 1 ml DNA complex, added to T75 flask and incubated overnight at 37° C. in 5% CO₂ incubator.
4. Appropriate serial dilutions of test compound were prepared and 5 μl/well transferred to the assay plate (white opaque CulturPlate-384, Perkin-Elmer) using Bravo (Agilent) with 5 μl NanoBRET Tracer K-5 (Promega N2501) diluted to the recommended concentration in assay buffer (Invitrogen 11058021) and 30 μl cell suspension. The plate was incubated for 2 h at 37° C. in 5% CO₂ incubator.
5. 20 μl 3× complete substrate plus inhibitor solution (containing NanoBRET Nano-Glo substrate and extracellular NanoLuc inhibitor diluted to manufacturer's recommendations in assay medium) was added to each well.
6. Donor emission wavelength (450 nm) and acceptor emission wavelength (610 nm or 630 nm) were measured on the Envision (Perkin-Elmer) and BRET ratio calculated for data analysis: BRET Ratio=(Acceptorₛₐₘₚₗₑ/Donorₛₐₘₚₗₑ)×1,000.
7. All assay plates contained 32 wells designated as 0% inhibition control wells, which were treated with vehicle only (1% DMSO) and 32 wells designated as 100% inhibition control wells, which were treated with a high concentration of non-specific kinase inhibitor in 1% DMSO. Percent inhibition in each test well was calculated using the formula (MEAN_{0% inhibition control wells}−test well reading)/(MEAN_{0% inhibition control wells}−MEAN_{100%inhibition control wells})×100%.
8. IC₅₀ was determined using a standard 4-parameter fit method (Model 205, XL-fit).

### Biological Data:

| | | | Assay 3: |
|---|---|---|---|
| | | | NanoBRET ™ |
| | Assay 1: | Assay 2: | assay for binding |
| Example | CK2α Enzyme | CLK2 Enzyme | to intracellular |
| No | IC₅₀ (nM) | IC₅₀ (nM) | CK2α (nM) |
| 1 | 1.38 | 226.3 | 124.0 |
| 2 | 12.50 | 1234.0 | 5879.0 |
| 3 | 0.77 | 33.4 | 134.7 |
| 4 | 3.63 | 1009.0 | 241.8 |
| 5 | 2.15 | 306.5 | 239.9 |
| 6 | 0.19 | 235.1 | 50.8 |
| 7 | 0.25 | 69.8 | 146.4 |
| 8 | 0.62 | 1097.0 | 303.7 |
| 9 | 7.69 | 568.1 | 8550.0 |
| 10 | 6.96 | 132.5 | 449.0 |
| 11 | 0.29 | 31.7 | 249.3 |
| 12 | 0.60 | 612.4 | 6828.0 |
| 13 | 1.43 | 51.7 | 3894.0 |
| 14 | 1.60 | 322.4 | 2116.0 |
| 15 | 27.12 | 1833.0 | >50000 |
| 16 | 14.12 | 371.0 | 3399.0 |
| 17 | 8.26 | 2781.0 | 3845.0 |
| 18 | 2.81 | 1623.0 | 4420.0 |
| 19 | 3.13 | 574.1 | 5069.0 |
| 20 | 23.51 | 157.7 | 3295.0 |
| 21 | 2.93 | 446.1 | 346.5 |
| 22 | 1.35 | 1122.0 | 363.0 |
| 23 | 19.91 | 86.1 | 17500.0 |
| 24 | 0.78 | 344.4 | 591.0 |
| 25 | 0.44 | 18.2 | 93.6 |
| 26 | 20.43 | 4462.0 | 875.2 |
| 27 | 0.95 | 56.4 | 321.6 |
| 28 | 0.54 | 107.0 | 70.6 |
| 29 | 22.14 | 648.9 | 2088.0 |
| 30 | 0.99 | 2562.0 | 816.5 |
| 31 | 0.32 | 526.4 | 753.4 |
| 32 | 0.47 | 75.9 | 17.7 |
| 33 | 0.48 | 77.8 | 452.2 |
| 34 | 3.32 | 70.8 | 9891.0 |
| 35 | 17.54 | 112.4 | >50000 |
| 36 | 1.39 | 48.9 | 1396.0 |
| 37 | 0.78 | 30.7 | 959.8 |
| 38 | 0.53 | 121.2 | 791.7 |
| 39 | 0.28 | 63.3 | 2078.0 |
| 40 | 9.06 | 87.5 | >50000 |
| 41 | 0.58 | 78.1 | 7622.0 |
| 42 | 6.94 | 83.8 | >50000 |
| 43 | 0.62 | 38.4 | 20250.0 |
| 44 | 1.46 | 39.6 | 16760.0 |
| 45 | 0.95 | 123.4 | 6377.0 |
| 46 | 2.65 | 237.1 | 13360.0 |
| 47 | 2.65 | 133.3 | 11950.0 |
| 48 | 20.25 | 992.9 | 2746.0 |
| 49 | 0.44 | 90.5 | 106.2 |
| 50 | 2.20 | 94.0 | 152.8 |
| 51 | 0.71 | 224.8 | 72.1 |
| 52 | 1.43 | 38.4 | 294.1 |
| 53 | 0.33 | 118.5 | 158.7 |
| 54 | 1.44 | 217.2 | 128.4 |
| 55 | 1.25 | 4736.0 | 148.9 |
| 56 | 6.59 | 3150.0 | 387.0 |
| 57 | 0.60 | 781.0 | 69.8 |
| 58 | 0.28 | 40.9 | 9294.0 |
| 59 | 132.00 | 21840.0 | >50000 |
| 60 | 215.20 | 30170.0 | |
| 61 | 103.60 | 3472.0 | 176.0 |
| 62 | 181.60 | 14270.0 | 29590.0 |
| 63 | 68.31 | 2370.0 | 4982.0 |
| 64 | 7.88 | 349.5 | 1845.0 |
| 65 | 163.10 | 4162.0 | >50000 |
| 66 | 176.40 | 1004.0 | 15570.0 |
| 67 | 277.80 | 4160.0 | 13780.0 |
| 68 | 136.50 | 566.2 | 18660.0 |
| 69 | 46.63 | 688.0 | 2846.0 |
| 70 | 255.50 | 1520.0 | >50000 |
| 71 | 1.37 | 415.9 | 2163.0 |
| 72 | 0.70 | 431.0 | 294.4 |
| 73 | 44.28 | 1957.0 | 742.7 |
| 74 | 19.66 | 10030.0 | 2128.0 |
| 75 | 0.89 | 403.5 | 76.5 |
| 76 | 2.64 | 18.6 | 735.2 |
| 77 | 5.71 | 95.7 | >50000 |
| 78 | 471.90 | >50000 | >50000 |
| 79 | 0.33 | 406.6 | 5086.0 |
| 80 | 46.72 | 1918.0 | 8050.0 |
| 81 | 7.49 | 684.4 | 807.6 |
| 82 | 23.28 | 1868.0 | 2121.0 |
| 83 | 2.56 | 1091.0 | 2260.0 |
| 84 | 395.40 | 10490.0 | 36470.0 |
| 85 | 743.00 | 37060.0 | >50000 |
| 86 | 6.36 | 1675.0 | 1351.0 |
| 87 | 12.33 | 1763.0 | 1296.0 |
| 88 | >1000 | 41320.0 | >50000 |
| 89 | >1000 | 18380.0 | >50000 |
| 90 | 212.10 | 8776.0 | 10550.0 |
| 91 | 4.17 | 979.3 | 4588.0 |
| 92 | 19.09 | 24.1 | 1777.0 |
| 93 | 0.77 | 568.8 | 179.8 |
| 94 | 202.60 | 5791.0 | 29250.0 |
| 95 | 6.56 | 203.2 | 335.8 |
| 96 | 26.99 | 226.8 | 1999.0 |
| 97 | 1.42 | 104.4 | 321.3 |
| 98 | 0.82 | 122.1 | 105.2 |
| 99 | 20.14 | 27990.0 | 1011.0 |
| 100 | 22.32 | 22920.0 | 1409.0 |
| 101 | 223.30 | 48610.0 | >50000 |
| 102 | 392.60 | 42980.0 | >50000 |
| 103 | 26.51 | 128.6 | 10660.0 |
| 104 | 64.16 | 264.0 | 14880.0 |
| 105 | 21.55 | 167.5 | 5335.0 |
| 106 | 41.69 | 368.1 | 26630.0 |
| 107 | 28.15 | 2638.0 | 3741.0 |
| 108 | 20.86 | 2734.0 | 2377.0 |
| 109 | 42.45 | 2918.0 | 2807.0 |
| 110 | 384.40 | 2721.0 | 20630.0 |
| 111 | 227.80 | 2304.0 | 14230.0 |
| 112 | 204.70 | 632.9 | 13980.0 |
| 113 | 67.51 | 2707.0 | 8088.0 |
| 114 | 50.53 | 2146.0 | 2524.0 |
| 115 | 115.20 | 6462.0 | 9907.0 |
| 116 | 246.70 | >50000 | >50000 |
| 117 | 3.00 | 1265.0 | 214.3 |
| 118 | 0.86 | 1105.0 | 204.4 |
| 119 | 22.65 | 616.7 | 1618.0 |
| 120 | 84.56 | 140.7 | 3248.0 |
| 121 | 44.91 | 209.1 | 3671.0 |
| 122 | 29.78 | 106.5 | 840.3 |
| 123 | 305.90 | 44740.0 | >50000 |
| 124 | 7.44 | 133.7 | 1993.0 |
| 125 | 161.50 | 151.9 | 49220.0 |
| 126 | 11.05 | 18080.0 | 3631.0 |
| 127 | 5.06 | 1382.0 | 306.1 |
| 128 | 3.96 | 187.4 | >50000 |
| 129 | 23.53 | 494.1 | >50000 |
| 130 | 105.90 | 325.8 | 15370.0 |
| 131 | 28.08 | 146.2 | 3003.0 |
| 132 | 10.90 | 441.6 | 868.8 |
| 133 | 31.55 | 2041.0 | 3067.0 |
| 134 | 5.25 | 2676.0 | 808.9 |
| 135 | 13.33 | 230.1 | 1830.0 |
| 136 | 33.08 | 123.8 | 3351.0 |
| 137 | 13.04 | 332.7 | 2873.0 |
| 138 | 33.68 | 114.8 | 5923.0 |
| 139 | 10.88 | 498.3 | 2361.0 |
| 140 | 10.22 | 2025.0 | 2801.0 |
| 141 | 2.77 | 17830.0 | 2055.0 |
| 142 | 5.43 | 4791.0 | 3861.0 |
| 143 | 7.01 | 624.7 | 563.9 |
| 144 | 4.72 | 270.7 | 346.8 |
| 145 | 25.42 | 185.9 | 1726.0 |
| 146 | 8.18 | 152.2 | 299.1 |
| 147 | 8.80 | 299.8 | 528.0 |
| 148 | 138.50 | >50000 | >50000 |
| 149 | 255.10 | 15300.0 | >50000 |
| 150 | 338.40 | 17680.0 | 39270.0 |
| 151 | 186.60 | 39800.0 | 19300.0 |
| 152 | 49.68 | 5821.0 | >50000 |
| 153 | 136.40 | 11650.0 | 34250.0 |
| 154 | 170.40 | 24720.0 | >50000 |
| 155 | 59.81 | >50000 | 2957.0 |
| 156 | 0.64 | 71.0 | 107.6 |
| 157 | 9.06 | 1649.0 | 827.8 |
| 158 | 10.96 | 613.6 | 737.7 |
| 159 | 1.93 | 64.3 | 703.3 |
| 160 | 19.06 | 221.1 | 1106.0 |
| 161 | 2.02 | 667.1 | 193.5 |
| 162 | 6.53 | 806.7 | 402.2 |
| 163 | 216.90 | 6571.0 | 26780.0 |
| 164 | 2.98 | 242.1 | 134.5 |
| 165 | 72.87 | 7325.0 | 3736.0 |
| 166 | 21.30 | 783.3 | 2674.0 |
| 167 | 21.04 | 774.4 | 830.6 |
| 168 | 0.64 | 81.2 | 208.1 |
| 169 | 79.15 | 5682.0 | 6511.0 |
| 170 | 6.64 | 6390.0 | >50000 |
| 171 | 45.28 | 23340.0 | 4898.0 |
| 172 | 125.70 | 44610.0 | >50000 |
| 173 | 1.17 | 3175.0 | 361.2 |
| 174 | 7.41 | 5976.0 | 4638.0 |
| 175 | 10.67 | 1914.0 | 1239.0 |
| 176 | 5.99 | 331.8 | 216.9 |
| 177 | 56.18 | 1251.0 | 11730.0 |
| 178 | 23.89 | 1496.0 | 2540.0 |
| 179 | 42.73 | 1930.0 | 1453.0 |
| 180 | 3.68 | 190.3 | 100.0 |
| 181 | 17.03 | 447.9 | 11390.0 |
| 182 | 29.62 | 1824.0 | 5411.0 |
| 183 | 2.39 | 906.7 | 144.7 |
| 184 | 0.94 | 17.0 | 177.2 |
| 185 | 0.76 | 787.1 | 106.3 |
| 186 | 717.00 | 4751.0 | >48880 |
| 187 | 8.62 | 4680.0 | 948.8 |
| 188 | 0.50 | 52.9 | 515.6 |
| 189 | 19.24 | 3496.0 | 1634.0 |
| 190 | 1.45 | 1102.0 | 74.0 |
| 191 | 0.57 | 132.6 | 842.0 |
| 192 | 10.63 | 664.8 | 377.1 |
| 193 | 1.21 | 354.9 | 87.1 |
| 194 | 0.76 | 194.8 | 14.9 |
| 195 | 0.41 | 226.8 | 21.4 |
| 196 | 12.40 | 300.5 | 54.7 |
| 197 | >1000 | >50000 | >3000 |
| 198 | 72.63 | >50000 | >3000 |
| 199 | 21.51 | 1213.0 | 1941.0 |
| 200 | 38.16 | 594.1 | 2960.0 |
| 201 | 0.87 | 7831.0 | 141.7 |
| 202 | 216.00 | 23340.00 | >3000 |
| 203 | 36.13 | 307.40 | 546.40 |
| 204 | 46.20 | 3442.00 | 405.20 |
| 205 | 12.72 | 213.90 | 467.50 |
| 206 | 63.85 | 2264.00 | 595.10 |
| 207 | 1.71 | 240.00 | 8.66 |
| 208 | 8.92 | 69.73 | 69.93 |

### REFERENCES

Battistutta & Lolli (2011). Structural and functional determinants of protein kinase CK2α: facts and open questions. Mol. Cell. Biochem., 2011, 356, 67-73.
Niefind et al (2001). Crystal structure of human protein kinase CK2: insights into basic properties of the CK2 holoenzyme. EMBO J. 2001, 20, 5320-5331.
Meggio & Pinna (2003). One-thousand-and-one substrates of protein kinas CK2. The FASEB Journal 17, 349-368.
Behan et al (2019). Prioritization of cancer therapeutic targets using CRISPR-Cas9 screens. Nature 568, 511-516.
Lin et al (2011). Overexpression of Nuclear Protein Kinase CK2α Catalytic Subunit (CK2α) as a Poor Prognosticator in Human Colorectal Cancer. PLoS ONE 6, 17193-
Ortega et al (2014). Mining CK2 in cancer. PLoS ONE 9, 115609-
Di Maira et al. (2019). The protein kinase CK2 contributes to the malignant phenotype of cholangiocarcinoma cells. Oncogenesis 8, 61-
Zhan et al (2017). Wnt signaling in cancer. Oncogene 36, 1461-1473.
Gao & Wang (2006). Casein Kinase 2 is activated and essential for Wnt/R-Catenin signaling.
Journal of Biological Chemistry 281, 189394-18400.
Dowling et al (2016). Potent and selective CK2 Kinase Inhibitors with effects on Wnt pathway signaling in vivo. ACS Med. Chem. Lett. 7, 300-305.
Zakharia et al. (2019). Pre-clinical in vitro and in vivo evidence of an antitumour effect of CX-4945, a casein kinase II inhibitor, in cholangiocarcinoma. Translational Oncology 12, 143-153.
Brear et al. (2016). Specific inhibition of CK2α from an anchor outside the active site. Chem. Sci. 7, 6839-6845.
Ruzzene & Pinna (2010). Addiction to protein kinase CK2: A common denominator of diverse cancer cells?Biochimica et Biophysica Acta 1804, 499-504.
Montenarh (2016). Protein Kinase CK2 in DNA Damage and repair. Transl. Cancer Res. 5, 49-63.
Gordon, D. E., Jang, G. M., Bouhaddou, M. et al. (2020). A SARS-CoV-2 protein interaction map reveals targets for drug repurposing. Nature 583, 459-468.

1. A compound of formula I, or a pharmaceutically acceptable salt, hydrate or solvate thereof: 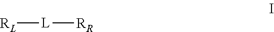
wherein:
L is linker that separates R_{L} from R_{R} by 7 to 13 bond lengths;
R_{L} is selected from one of formulae Ic, Id, Ie, If, Ig, Ih, Ii, Ij or Ik shown below: 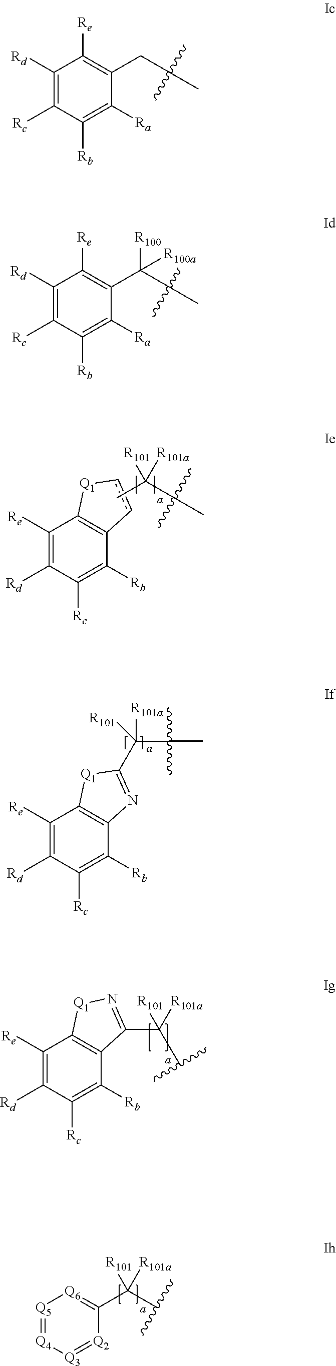 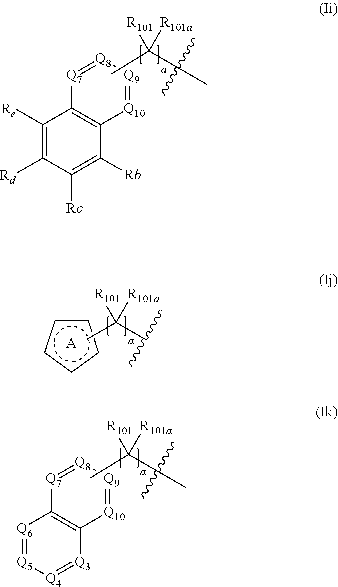
wherein:
Rₐ and Rₑ are each independently selected from hydrogen, methyl or halo;
R_{b} and R_{d} are each independently selected from hydrogen, halo, cyano, (1-4C)alkyl, (3-6C)cycloalkyl,
—[CH₂]₀₋₃-(1-4C)alkoxy,
—[CH₂]₀₋₃—C(O)NH₂,
—[CH₂]₀₋₃—C(O)NH(1-4C)alkyl,
—[CH₂]₀₋₃—C(O)N[(1-4C)alkyl]₂,
—[CH₂]₀₋₃—NH₂,
—[CH₂]₀₋₃—NH(1-4C)alkyl,
—[CH₂]₀₋₃—N[(1-4C)alkyl]₂,
—[CH₂]₀₋₃—S(O)_{q}-(1-4C)alkyl (wherein q is 0, 1 or 2),
—[CH₂]₀₋₃—C(O)(1-4C)alkyl,
—[CH₂]₀₋₃—C(O)OH,
—[CH₂]₀₋₃—C(O)O-(1-4C)alkyl,
—[CH₂]₀₋₃—N(R_{f})C(O)-(1-4C)alkyl (wherein R_{f} is hydrogen or methyl),
—[CH₂]₀₋₃—S(O)₂NH(1-4C)alkyl,
—[CH₂]₀₋₃—S(O)₂N[(1-4C)alkyl]₂,
—[CH₂]₀₋₃—N(R₉)SO₂-(1-4C)alkyl (wherein R₉ is hydrogen or methyl),
a group of the formula: 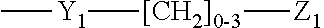
wherein Y₁ is absent, —O—, —NH—, —NMe-, —S—, —S(O)— or —S(O)₂—; and
Z₁ is (3-6C)cycloalkyl, phenyl, a 4- to 6-membered heterocyclyl or 5 or 6-membered heteroaryl;
and wherein:
any alkyl, alkoxy, cycloalkyl or —[CH₂]— moiety within a R_{b} and R_{d} substituent group is optionally substituted by one or more substituents selected from halo, hydroxy, cyano, amino, —C(O)OH, —C(O)NH₂, (1-2C)alkoxy, or (3-4C)cycloalkoxy; and
Z₁ is optionally substituted by one or more substituents selected from: halo, hydroxy, cyano, amino, —C(O)OH, —C(O)NH₂, (1-2C)alkoxy, (1-2C)alkyl, (3-4C)cycloalkyl, (3-4C)cycloalkoxy, —C(O)NH(1-2C)alkyl, —C(O)N[(1-2C)alkyl]₂, —NH(1-2C)alkyl, -N[(1-2C)alkyl]₂, —S(O)_{q}-(1-2C)alkyl (wherein q is 0, 1 or 2), —C(O)(1-2C)alkyl, —C(O)O-(1-2C)alkyl, —N(R_{f})C(O)-(1-2C)alkyl, —S(O)₂NH(1-2C)alkyl, —S(O)₂N[(1-2C)alkyl]₂, or —NHSO₂-(1-2C)alkyl, and wherein any (1-2C)alkoxy, (1-2C)alkyl, (3-4C)cycloalkyl or (3-4C)cycloalkoxy group is optionally substituted by one or more substituents selected from halo, cyano, hydroxy, (1-2C)alkyl, (1-2C)alkoxy or (1-2C)alkoxy-(1-2C)alkyl;
R_{c} is selected from hydrogen, halo, cyano, —C(O)NH₂, (1-4C)alkyl, (3-6C)cycloalkyl
—[CH₂]₀₋₃-(1-4C)alkoxy,
—[CH₂]₀₋₃-(3-6C)cycloalkoxy,
—[CH₂]₀₋₃—C(O)NH₂,
—[CH₂]₀₋₃—C(O)NH(1-4C)alkyl,
—[CH₂]₀₋₃—C(O)N[(1-4C)alkyl]₂,
—[CH₂]₀₋₃—NH₂,
—[CH₂]₀₋₃—NH(1-4C)alkyl,
—[CH₂]₀₋₃—N[(1-4C)alkyl]₂,
—[CH₂]₀₋₃—S(O)_{q}-(1-4C)alkyl (wherein q is 0, 1 or 2),
—[CH₂]₀₋₃—C(O)(1-4C)alkyl,
—[CH₂]₀₋₃—C(O)OH,
—[CH₂]₀₋₃—C(O)O-(1-4C)alkyl,
—[CH₂]₀₋₃—N(R_{b})C(O)-(1-4C)alkyl (wherein Rₕ is hydrogen or methyl),
—[CH₂]₀₋₃—S(O)₂NH(1-4C)alkyl,
—[CH₂]₀₋₃—S(O)₂N[(1-4C)alkyl]₂,
—[CH₂]₀₋₃—N(Ri)SO₂-(1-4C)alkyl (wherein Ri is hydrogen or methyl),
a group of the formula: 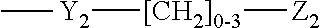
wherein Y₂ is absent, —O—, —NH—, —NMe-, —S—, —S(O)— or —S(O)₂—; and
Z₂ is (3-6C)cycloalkyl, phenyl, a 4- to 6-membered heterocyclyl or 5 or 6-membered heteroaryl;
and wherein:
any alkyl, alkoxy, cycloalkyl or —[CH₂]— moiety within a R_{c} substituent group is optionally substituted by one or more substituents selected from halo, hydroxy, cyano, amino, —C(O)OH, —C(O)NH₂, (1-2C)alkoxy, or (3-4C)cycloalkoxy; and
Z₂ is optionally substituted by one or more substituents selected from: halo, hydroxy, cyano, amino, —C(O)OH, —C(O)NH₂, (1-2C)alkoxy, (1-2C)alkyl, (3-4C)cycloalkyl, (3-4C)cycloalkoxy, —C(O)NH(1-2C)alkyl, —C(O)N[(1-2C)alkyl]₂, —NH(1-2C)alkyl, -N[(1-2C)alkyl]₂, —S(O)_{q}-(1-2C)alkyl (wherein q is 0, 1 or 2), —C(O)(1-2C)alkyl, —C(O)O-(1-2C)alkyl, —N(R_{f})C(O)-(1-2C)alkyl, —S(O)₂NH(1-2C)alkyl, —S(O)₂N[(1-2C)alkyl]₂, or —NHSO₂-(1-2C)alkyl, and wherein any (1-2C)alkoxy, (1-2C)alkyl, (3-4C)cycloalkyl or (3-4C)cycloalkoxy group is optionally substituted by one or more substituents selected from halo, cyano, hydroxy, (1-2C)alkyl, (1-2C)alkoxy or (1-2C)alkoxy-(1-2C)alkyl;
R₁₀₀ is (1-2C)alkyl optionally substituted by hydroxy or halo;
R₁₀₀ₐ is selected from hydrogen or methyl;
or R₁₀₀ and R₁₀₀ₐ are linked to form a cyclopropyl or cyclobutyl ring;
R₁₀₁ is hydrogen or (1-2C)alkyl optionally substituted by hydroxy or halo;
R₁₀₁ₐ is selected from hydrogen or methyl;
or R₁₀₁ and R₁₀₁ₐ are linked to form a cyclopropyl or cyclobutyl ring;
integer a is 0, 1 or 2;
Q₁ is selected from —NR₁₀₂—, —O—, —S— or —CH—;
R₁₀₂ is hydrogen or (1-2C)alkyl; [symbol]
represents a single or double bond;
Q₂ is N or CRₐ;
Q₃ is N or CR_{b};
Q₄ is N or CR_{c};
Q₅ is N or CR_{d};
Q₆ is N or CRₑ;
Rₐ, R_{b}, R_{c}, R_{d} and Rₑ are each as defined above;
with the proviso that one to three of Q₂, Q₃, Q₄, Q₅ or Q₆ is/are N;
Q₇ is N or CR_{f};
Q₈ is N or CR_{f};
Q₉ is N or CR_{f};
Q₁₀ is N or CR_{f};
with the proviso that one or two of Q₇, Q₈, Q₉ or Q₁₀ is/are N;
each R_{f} present is independently selected from hydrogen, methyl or halo;
Ring A is a five-membered heteroaryl ring optionally substituted by one R_{b} and/or one or two R_{c} substituents;
R_{R} is selected from one of formulae Im, In or Io shown below: 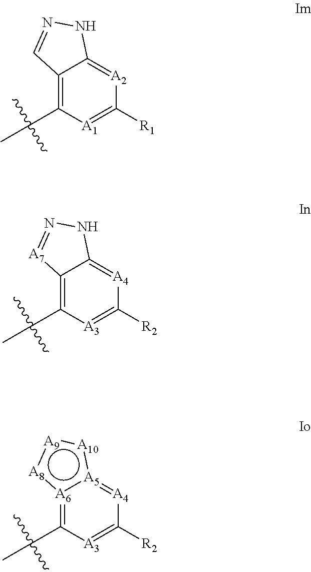
wherein: [symbol]
denotes the point of attachment;
A₁ and A₂ are both CH; or one of A₁ and A₂ is N and the other is CH;
R₁ is a 5- or 6-membered heteroaryl ring which is optionally substituted on any available carbon atom by one or more R_{1A} substituent groups and on any available nitrogen atom by one or more R_{1B} substituent groups; and wherein:
each R_{1A} group present is selected from hydroxy, cyano, amino, halo, —C(O)OH, —C(O)NH₂, (1-2C)alkoxy, (1-2C)alkyl, (3-4C)cycloalkyl, (3-4C)cycloalkoxy, —C(O)NH(1-2C)alkyl, —C(O)N[(1-2C)alkyl]₂, —NH(1-2C)alkyl, —N[(1-2C)alkyl]₂, —S(O)_{q}-(1-2C)alkyl (wherein q is 0, 1 or 2), —C(O)(1-2C)alkyl, —C(O)O-(1-2C)alkyl, —N(R_{f})C(O)-(1-2C)alkyl (wherein R_{f} is hydrogen or methyl), —S(O)₂NH(1-2C)alkyl, —S(O)₂N[(1-2C)alkyl]₂, or —NHSO₂-(1-2C)alkyl; and wherein any (1-2C)alkoxy, (1-2C)alkyl, (3-4C)cycloalkyl or (3-4C)cycloalkoxy group is optionally substituted by one or more substituents selected from halo, cyano, hydroxy, (1-2C)alkyl, (1-2C)alkoxy or (1-2C)alkoxy-(1-2C)alkyl];
and R_{1B} is (1-2C)alkyl or (3-4C)cycloalkyl;
A₃ is selected from CH, CR₃ or N;
A₄ is selected from CH, CR₄ or N;
A₅ and A₆ are both C, or one of A₅ and A₆ is N and the other is C;
A₇ is selected from CH, CR₇ or N;
R₂ is selected from:
(iii) a group R₁ defined above;
(iv) hydrogen, halo, cyano, (1-4C)alkyl, (1-4C)cyanoalkyl, (1-4C)hydroxyalkyl, (1-4C)haloalkyl, (1-4C)aminoalkyl,
—[CH₂]₀₋₃-(1-4C)alkoxy,
—[CH₂]₀₋₃—C(O)NH₂,
—[CH₂]₀₋₃—C(O)NH(1-4C)alkyl,
—[CH₂]₀₋₃—C(O)N[(1-4C)alkyl]₂,
—[CH₂]₀₋₃—NH(1-4C)alkyl,
—[CH₂]₀₋₃—N[(1-4C)alkyl]₂,
—[CH₂]₀₋₃—S(O)_{q}-(1-4C)alkyl (wherein q is 0, 1 or 2),
—[CH₂]₀₋₃—C(O)(1-4C)alkyl,
—[CH₂]₀₋₃—C(O)OH,
—[CH₂]₀₋₃—C(O)O-(1-4C)alkyl,
—[CH₂]₀₋₃—N(R₂ₐ)C(O)-(1-4C)alkyl (wherein R₂ₐ is hydrogen or methyl),
—[CH₂]₀₋₃—S(O)₂NH(1-4C)alkyl,
—[CH₂]₀₋₃—S(O)₂N[(1-4C)alkyl]₂,
—[CH₂]₀₋₃—N(R_{2b})SO₂-(1-4C)alkyl (wherein R_{2b} is hydrogen or methyl),
a 4- to 7-membered heterocyclyl,
(3-6C)cycloalkyl,
(3-6C)cycloalkyl(1-2C)alkyl,
phenyl, or
phenyl(1-2C)alkyl;
and wherein any cycloalkyl, heterocyclyl or phenyl group present is optionally substituted by one or more hydroxy, cyano, amino, halo, —C(O)OH, —C(O)NH₂, (1-2C)alkoxy, (1-2C)alkyl, —C(O)NH(1-2C)alkyl, —C(O)N[(1-2C)alkyl]₂, —NH(1-2C)alkyl, —N[(1-2C)alkyl]₂, —S(O)_{q}-(1-2C)alkyl (wherein q is 0, 1 or 2), —C(O)(1-2C)alkyl, —C(O)O-(1-2C)alkyl, —N(R_{f})C(O)-(1-2C)alkyl (wherein R_{f} is hydrogen or methyl), —S(O)₂NH(1-2C)alkyl, —S(O)₂N[(1-2C)alkyl]₂, or —NHSO₂-(1-2C)alkyl;
R₃ is selected from fluoro, chloro, methyl, methoxy or cyano;
R₄ is selected from fluoro, chloro, methyl, methoxy or cyano;
R₇ is selected from methyl, —NH(1-2C)alkyl or —NH(3-4C)cycloalkyl;
and wherein R₂ is not a group R₁ when A₃, A₄ and A₇ are all CH and A₅ and A₆ are C;
A₈ is selected from CH, CR₈, O, S, NH or N;
A₉ is selected from CH, CR₉, O, S, NH or N;
A₁₀ is selected from CH, CR₁₀, O, S, NH or N;
R₈, R₉ and R₁₀ are selected from methyl, amino, —NH(1-2C)alkyl or —NH(3-4C)cycloalkyl;
and wherein:
(i) only one of A₈, A₈ and A₁₀ can be NH;
(ii) one to four of A₅, A₈, A₉ and A₁₀ or A₆, A₈, A₉ and A₁₀ can be N
(iii) only one of A₈, A₉ or A₁₀ can be O or S;
(iv) when one of A₈, A₈ or A₁₀ are O or S, A₅ and/or A₆ cannot be N.

2. A compound according to claim 1, or a pharmaceutically acceptable salt, hydrate or solvate thereof, wherein L is linker that separates R_{L} from R_{R} by 8 to 12 bond lengths, 9 to 11 bond lengths, or 10 bond lengths.

3. A compound according to any one of claims 1 or 2, or a pharmaceutically acceptable salt, hydrate or solvate thereof, wherein L is a linker selected from:
(ii) a group of the formula: 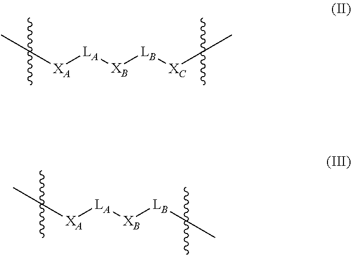
wherein:
X_{A} is selected from: —O—, —S—, —SO—, —SO₂—, —N(Rₓₐ)—, —C(O)—, —C(O)O—, —OC(O)—, —C(O)N(Rₓₐ)—, —N(Rₓₐ)C(O)—, —N(Rₓₐ₁)C(O)N(Rₓₐ)—, —S(O)₂N(Rₓₐ)— or —N(Rₓₐ)SO₂—, wherein Rₓₐ and Rₓₐ₁ are selected from hydrogen or methyl;
L_{A} is a (1-6C)alkylene optionally substituted by one or more R_{LA} substituent groups, or a -[(0-3C)alkylene-(3-6C)cycloalkylene-(0-3C)alkylene]-group optionally substituted by one or more R_{LA} substituent groups;
each R_{LA} group present is selected from hydroxy, halo, (1-2C)alkyl, (1-2C)hydroxyalkyl or (1-2C)haloalkyl;
X_{B} is selected from: —O—, —S—, —SO—, —SO₂—, —N(R_{xb})—, —C(O)—, —C(O)O—, —OC(O)—, —C(O)N(R_{xb})—, —N(R_{xb})C(O)—, —N(R_{xb})C(O)N(R_{xb})—, —S(O)₂N(R_{xb})— or —N(R_{xb})SO₂—, wherein R_{xb} and R_{xb1} are selected from hydrogen or methyl;
L_{B} is a (1-6C)alkylene optionally substituted by one or more R_{LB} substituent groups, or a -[(0-3C)alkylene-(3-6C)cycloalkylene-(0-3C)alkylene]-group optionally substituted by one or more R_{LB} substituent groups;
each R_{LB} group present is selected from hydroxy, halo, (1-2C)alkyl, (1-2C)hydroxyalkyl or (1-2C)haloalkyl;
X_{c} is selected from: —O—, —S—, —SO—, —SO₂—, —N(R_{xc})—, —C(O)—, —C(O)O—, —OC(O)—, —C(O)N(R_{xc})—, —N(R_{xc1})C(O)—, —N(R_{xc1})C(O)N(R_{xc})—, —S(O)₂N(R_{xc})— or —N(R_{xc})SO₂—, wherein R_{xc} and R_{xc1} are selected from hydrogen or methyl;
(ii) a group of the formula: 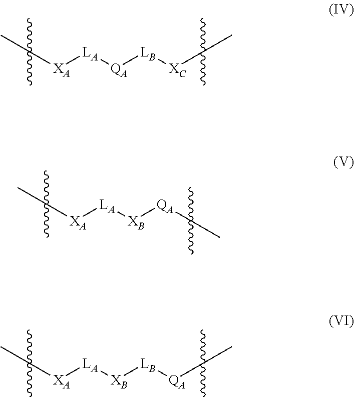
wherein:
X_{A}, L_{A}, X_{B}, L_{B} and X_{C} are as defined above;
Q_{A} is selected from a 5 or 6-membered heteroaryl ring, a 9 or 10-membered bicyclic heteroaryl ring, (3-6C)cycloalkyl, a phenyl, or a 4 to 6 membered heterocyclic ring;
(iii) a group of the formula: 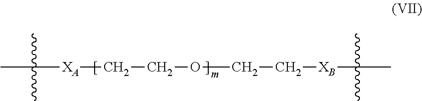
wherein:
X_{A} and X_{B} are each as defined above;
integer m is 1 or 2.

4. A compound according to any one of the preceding claims, or a pharmaceutically acceptable salt, hydrate or solvate thereof, wherein L is a linker selected from:
(i) a group of the formula: 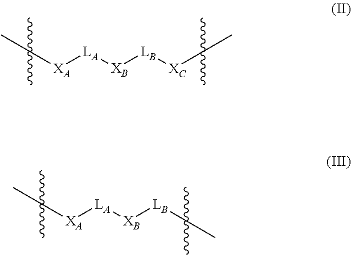
wherein:
X_{A} is selected from: —O—, —SO₂—, —N(Rₓₐ)—, —C(O)—, —C(O)N(Rₓₐ)—, —N(Rₓₐ)C(O)—, —S(O)₂N(Rₓₐ)— or —N(Rₓₐ)SO₂—, wherein Rₓₐ is selected from hydrogen or methyl;
L_{A} is a (1-5C)alkylene optionally substituted by one or more R_{LA} substituent groups, or a -[(0-2C)alkylene-(3-6C)cycloalkylene-(0-2C)alkylene]-group optionally substituted by one or more R_{LA} substituent groups;
each R_{LA} group present is selected from (1-2C)alkyl, (1-2C)hydroxyalkyl or (1-2C)haloalkyl;
X_{B} is selected from: —O—, —SO₂—, —N(R_{xb})—, —C(O)—, —C(O)N(R_{xb})—, —N(R_{xb1})C(O)—, —S(O)₂N(R_{xb})— or —N(R_{xb})SO₂—, wherein R_{xb} and R_{xb1} are selected from hydrogen or methyl;
L_{B} is a (1-4C)alkylene optionally substituted by one or more R_{LB} substituent groups, or a -[(0-2C)alkylene-(3-6C)cycloalkylene-(0-2C)alkylene]-group optionally substituted by one or more R_{LB} substituent groups;
each R_{LB} group present is selected from (1-2C)alkyl, (1-2C)hydroxyalkyl or (1-2C)haloalkyl;
X_{c} is selected from: —O—, —SO₂—, —N(R_{xc})—, —C(O)—, —C(O)N(R_{xc})—, —N(R_{xc1})C(O)—, —S(O)₂N(R_{xc})— or —N(R_{xc})SO₂—, wherein R_{xc} and R_{xc1} are selected from hydrogen or methyl;
(ii) a group of the formula: 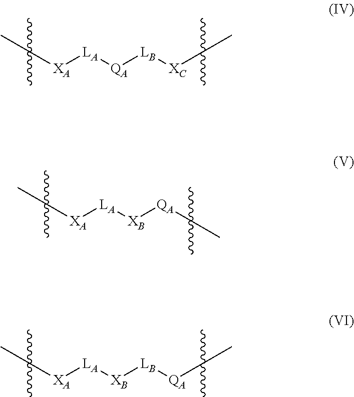
wherein:
X_{A}, L_{A}, X_{B}, L_{B} and X_{C} are as defined above;
Q_{A} is selected from a 5 or 6-membered heteroaryl ring, or a 4 to 6 membered heterocyclic ring;
(iii) a group of the formula: 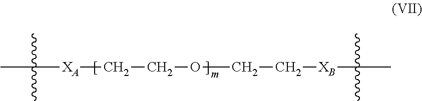
wherein:
X_{A} and X_{B} are each as defined above:
integer m is 1.

5. A compound according to any one of the preceding claims, or a pharmaceutically acceptable salt, hydrate or solvate thereof, wherein L is a linker selected from:
(i) a group of the formula: 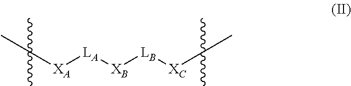
wherein:
X_{A} is selected from: —O—, —N(Rₓₐ)—, —C(O)N(Rₓₐ)—, or —N(Rₓₐ)C(O)—, wherein Rₓₐ is selected from hydrogen or methyl;
L_{A} is a (2-5C)alkylene optionally substituted by one or more R_{LA} substituent groups, or a -[(4-6C)cycloalkylene-(0-1C)alkylene]-group, each RLA group present is (1-2C)alkyl;
X_{B} is selected from: —O—, —N(R_{xb})—, —C(O)N(R_{xb})—, or —N(R_{xb1})C(O)—, wherein R_{xb} and R_{xb1} are selected from hydrogen or methyl;
L_{B} is a (1-4C)alkylene optionally substituted by one or more R_{LB} substituent groups,
each R_{LB} group present is (1-2C)alkyl;
X_{c} is selected from: —O—, —N(R_{xc})—, —C(O)N(R_{xc})— or —N(R_{xc1})C(O)—, wherein R_{xc} and R_{xc1} are selected from hydrogen or methyl;
(ii) a group of the formula: 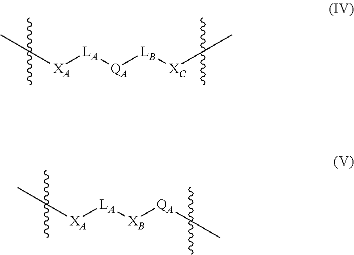
wherein:
X_{A}, L_{A}, X_{B}, L_{B} and X_{C} are as defined above;
Q_{A} is selected from a 5 or 6-membered heteroaryl ring, or a 4 to 6 membered heterocyclic ring.

6. A compound according to any one of the preceding claims, or a pharmaceutically acceptable salt, hydrate or solvate thereof, wherein L is a linker selected from:
(i) a group of the formula: 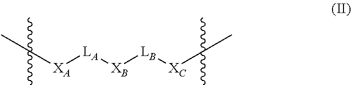
wherein:
X_{A} is selected from: —N(H)—, or —N(Me)-;
L_{A} is a (2-4C)alkylene optionally substituted by a methyl group, or L_{A} is a -[cyclobutylene-(0-1C)alkylene]-group;
X_{B} is selected from: —O—, —N(H)—, —N(Me)-, —C(O)N(H)—, or —C(O)N(Me)-;
L_{B} is a (1-4C)alkylene;
X_{c} is selected from: —O—, —N(H)—, —N(Me)-, —C(O)N(H)— or —C(O)N(Me)-;
(ii) a group of the formula: 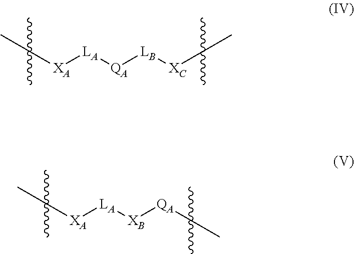
wherein:
X_{A}, L_{A}, X_{B}, L_{B} and X_{C} are as defined above;
Q_{A} is selected from an oxazole group, triazole group or a azetidine group.

7. A compound according to any one of the preceding claims, or a pharmaceutically acceptable salt, hydrate or solvate thereof, wherein L is a linker selected from:

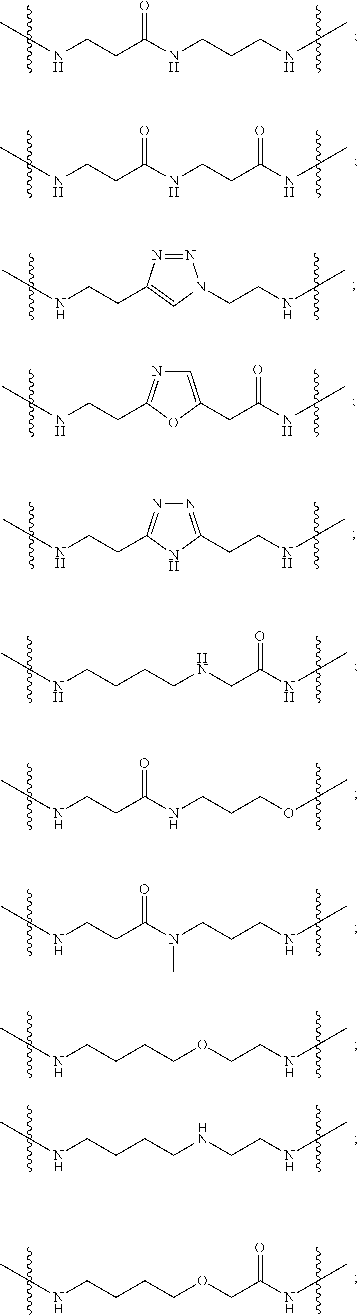

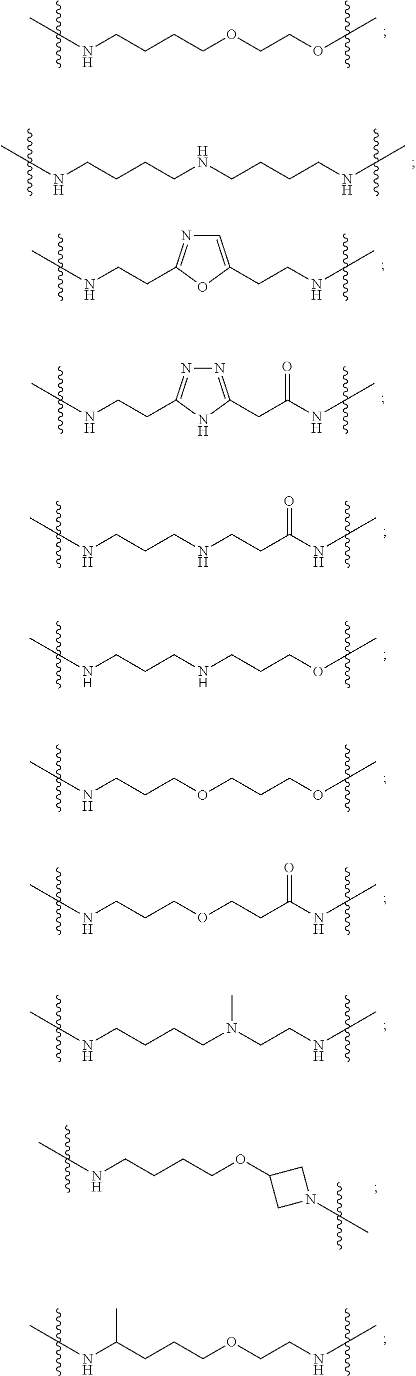

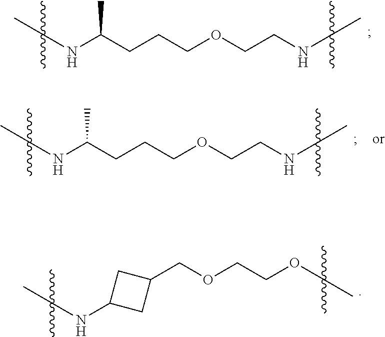 [SMILES: *NCCOCCC[C@H](C)N* ; *NCCOCCC[C@@H](C)N* ; *NC1CC(COCCO*)C1]

8. A compound according to any one of the preceding claims, or a pharmaceutically acceptable salt, hydrate or solvate thereof, wherein R_{L} is selected from one of formulae Ic, Ie or If shown below: 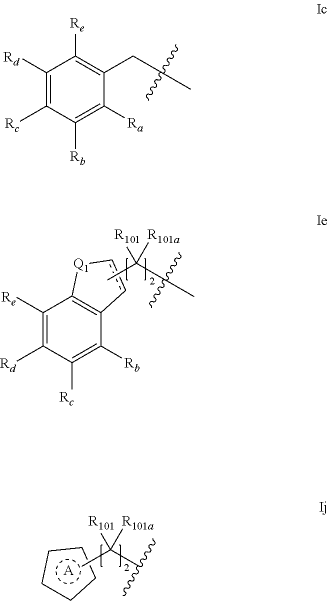
wherein: [symbol]
denotes the point of attachment;

9. A compound according to any one of the preceding claims, or a pharmaceutically acceptable salt, hydrate or solvate thereof, wherein R_{L} is selected from one of formulae shown below: 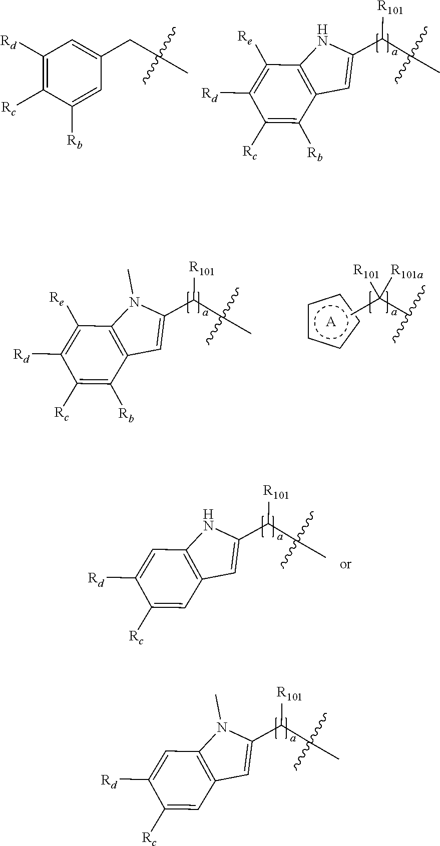
wherein: [symbol]
denotes the point of attachment.

10. A compound according to any one of the preceding claims, or a pharmaceutically acceptable salt, hydrate or solvate thereof, wherein Rₐ and Rₑ are both hydrogen.

11. A compound according to any one of the preceding claims, or a pharmaceutically acceptable salt, hydrate or solvate thereof, wherein R_{b} and R_{d} are each independently selected from hydrogen, halo, cyano, (1-4C)alkyl, (3-6C)cycloalkyl,
—[CH₂]₀₋₁-(1-4C)alkoxy,
—[CH₂]₀₋₁—C(O)NH₂,
—[CH₂]₀₋₁—C(O)NH(1-2C)alkyl,
—[CH₂]₀₋₁—C(O)(1-2C)alkyl,
—[CH₂]₀₋₁—C(O)OH,
a group of the formula: 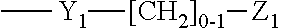
wherein Y₁ is absent, —O—, —NH—, —NMe-, —S—, —S(O)— or —S(O)₂—; and
Z₁ is (3-6C)cycloalkyl, phenyl, a 4- to 6-membered heterocyclyl or 5 or 6-membered heteroaryl;
and wherein:
any alkyl, alkoxy, cycloalkyl or —[CH₂]— moiety within a R_{b} and R_{d} substituent group is optionally substituted by one or more substituents selected from halo, hydroxy, cyano, amino, —C(O)OH, —C(O)NH₂, (1-2C)alkoxy, or (3-4C)cycloalkoxy; and
Z₁ is optionally substituted by one or more substituents selected from: halo, hydroxy, cyano, amino, —C(O)OH, —C(O)NH₂, (1-2C)alkoxy, (1-2C)alkyl, (3-4C)cycloalkyl, (3-4C)cycloalkoxy, —C(O)NH(1-2C)alkyl, —C(O)N[(1-2C)alkyl]₂, —NH(1-2C)alkyl, —N[(1-2C)alkyl]₂, —C(O)(1-2C)alkyl, —C(O)O-(1-2C)alkyl or —N(R_{f})C(O)—(1-2C)alkyl, and wherein any (1-2C)alkoxy, (1-2C)alkyl, (3-4C)cycloalkyl or (3-4C)cycloalkoxy group is optionally substituted by one or more substituents selected from halo, cyano, hydroxy, (1-2C)alkyl, (1-2C)alkoxy or (1-2C)alkoxy-(1-2C)alkyl.

12. A compound according to any one of the preceding claims, or a pharmaceutically acceptable salt, hydrate or solvate thereof, wherein one of R_{b} and R_{d} is hydrogen or halogen and the other is selected from hydrogen, fluoro, chloro cyano, methyl, ethyl, methoxy, ethoxy, —C(O)NH₂, —CH₂-oxazole, —CH₂OH, —CH₂CN, —CH₂CH₂OH, —CF3, —OCF₃, —O—CH₂CH₂OH or —CH₂—C(O)NH₂.

13. A compound according to any one of the preceding claims, or a pharmaceutically acceptable salt, hydrate or solvate thereof, wherein R_{c} is selected from hydrogen, halo, cyano, —C(O)NH₂, (1-4C)alkyl, (3-6C)cycloalkyl, -(1-4C)alkoxy,
a group of the formula: 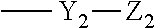
wherein Y₂ is absent, —O—, —NH— or —NMe-; and
Z₂ is (3-6C)cycloalkyl, phenyl, a 4- to 6-membered heterocyclyl or 5 or 6-membered heteroaryl;
and wherein:
any alkyl, alkoxy, cycloalkyl or —[CH₂]— moiety within a R_{c} substituent group is optionally substituted by one or more substituents selected from halo, hydroxy, cyano, amino, —C(O)OH, —C(O)NH₂, (1-2C)alkoxy, or (3-4C)cycloalkoxy; and
Z₂ is optionally substituted by one or more substituents selected from: halo, hydroxy, cyano, amino, —C(O)OH, —C(O)NH₂, (1-2C)alkoxy, (1-2C)alkyl, (3-4C)cycloalkyl, (3-4C)cycloalkoxy, —C(O)NH(1-2C)alkyl, —C(O)N[(1-2C)alkyl]₂, —NH(1-2C)alkyl, —N[(1-2C)alkyl]₂, —C(O)(1-2C)alkyl or —C(O)O-(1-2C)alkyl, and wherein any (1-2C)alkoxy, (1-2C)alkyl, (3-4C)cycloalkyl or (3-4C)cycloalkoxy group is optionally substituted by one or more substituents selected from halo, cyano, hydroxy, (1-2C)alkyl, (1-2C)alkoxy or (1-2C)alkoxy-(1-2C)alkyl.

14. A compound according to any one of the preceding claims, or a pharmaceutically acceptable salt, hydrate or solvate thereof, wherein R_{c} is selected from hydrogen, —CF₃, —OCF₃, cyclopropyl, phenyl, —O-cyclobutyl or -phenyl-CH₂—OH;

15. A compound according to any one of the preceding claims, or a pharmaceutically acceptable salt, hydrate or solvate thereof, wherein R_{R} is selected from one of formulae Im or In shown below: 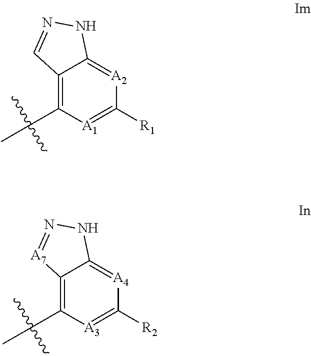
wherein: [symbol]
denotes the point of attachment;

16. A compound according to any one of the preceding claims, or a pharmaceutically acceptable salt, hydrate or solvate thereof, wherein R₁ is a 5- or 6-membered heteroaryl ring comprising one, two, three or four heteroatoms selected from N, O or S which is optionally substituted on any available carbon atom by one or more R_{1A} substituent groups and on any available nitrogen atom by one or more R_{1B} substituent groups;

17. A compound according to any one of the preceding claims, or a pharmaceutically acceptable salt, hydrate or solvate thereof, wherein R₁ is selected from: 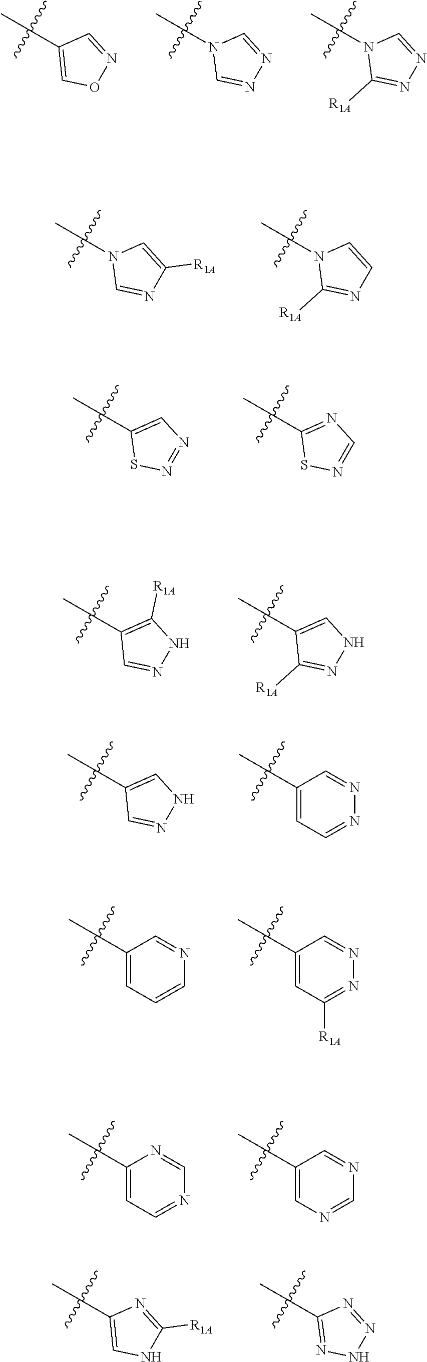 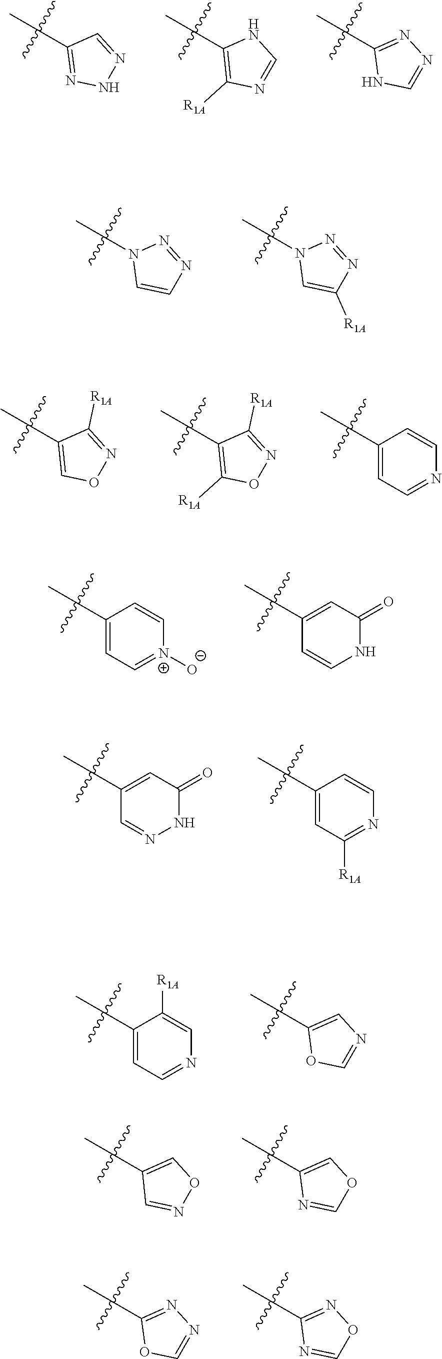
wherein R_{1A} is as defined herein;

18. A compound according to any one of the preceding claims, or a pharmaceutically acceptable salt, hydrate or solvate thereof, wherein each R_{1A} group present is selected from hydroxy, cyano, amino, chloro, fluoro, —C(O)OH, —C(O)NH₂, (1-2C)alkoxy, (1-2C)alkyl; and wherein any (1-2C)alkoxy, (1-2C)alkyl group is optionally substituted by one or more substituents selected from halo, cyano, hydroxy, (1-2C)alkyl, (1-2C)alkoxy or (1-2C)alkoxy-(1-2C)alkyl.

19. A compound according to any one of the preceding claims, or a pharmaceutically acceptable salt, hydrate or solvate thereof, wherein each R_{1B} group is methyl.

20. A compound according to any one of the preceding claims, or a pharmaceutically acceptable salt, hydrate or solvate thereof, wherein R₂ is selected from:
(i) a group R₁ defined above;
(ii) hydrogen, halo, cyano, (1-2C)alkyl, (1-2C)cyanoalkyl, (1-2C)hydroxyalkyl, (1-2C)haloalkyl, (1-2C)aminoalkyl,
—[CH₂]₀₋₁-(1-2C)alkoxy,
—[CH₂]₀₋₁—C(O)NH₂,
—[CH₂]₀₋₁—C(O)NH(1-2C)alkyl,
—[CH₂]₀₋₁—C(O)N[(1-2C)alkyl]₂,
—[CH₂]₀₋₁—C(O)(1-2C)alkyl,
—[CH₂]₀₋₂—C(O)OH,
—[CH₂]₀₋₁—C(O)O-(1-2C)alkyl,
—[CH₂]₀₋₁—N(H)C(O)—(1-2C)alkyl,
a 4- to 6-membered heterocyclyl,
(4-6C)cycloalkyl,
(4-6C)cycloalkyl(1-2C)alkyl,
phenyl, or
phenyl(1-2C)alkyl;
and wherein any cycloalkyl, heterocyclyl or phenyl group present is optionally substituted by one or more hydroxy, cyano, amino, halo, —C(O)OH, —C(O)NH₂, methoxy, methyl, —C(O)NH(Me), —C(O)N(Me)₂, —NH(Me), —N(Me)₂, —S(O)_{q}-(Me) (wherein q is 0, 1 or 2), —C(O)(Me), —C(O)O-(Me), —N(H)C(O)-(Me), —S(O)₂NH(Me), —S(O)₂N(Me)₂, or —NHSO₂-(Me).

21. A compound according to any one of the preceding claims, or a pharmaceutically acceptable salt, hydrate or solvate thereof, wherein R₂ is selected from:
(i) a group R₁ defined above;
(ii) hydrogen, cyano, —C(O)NH₂, —C(O)N(Me)₂, CH₂CH₂—C(O)OH, —C(O)OH, -morpholino, -oxetane, -tetrahydrofuran or -tetrahydropyran.

22. A compound according to any one of the preceding claims, or a salt, hydrate or solvate thereof, wherein the compound is selected from any one of the following:
3-(((2-Chloro-[1,1′-biphenyl]-4-yl)methyl)amino)-N-(3-((6-(pyridin-4-yl)-1H-indazol-4-yl)amino)propyl)propanamide;
4-((3-(3-(((2-chloro-[1,1′-biphenyl]-4-yl)methyl)amino)propanamido)propyl)amino)-1H-indazole-6-carboxamide;
N-(3-((6-(1H-pyrazol-4-yl)-1H-indazol-4-yl)amino)propyl)-3-(((2-chloro-[1,1′-biphenyl]-4-yl)methyl)amino)propanamide;
N-(3-((6-(1H-1,2,4-triazol-1-yl)-1H-indazol-4-yl)amino)propyl)-3-(((2-chloro-[1,1′-biphenyl]-4-yl)methyl)amino)propanamide;
3-((3-Chloro-4-(trifluoromethoxy)benzyl)amino)-N-(3-((6-(pyridin-4-yl)-1H-indazol-4-yl)amino)propyl)propanamide;
3-(((2-chloro-[1,1′-biphenyl]-4-yl)methyl)amino)-N-(3-((6-(pyridazin-4-yl)-1H-indazol-4-yl)amino)propyl)propanamide;
3-(((2-chloro-[1,1′-biphenyl]-4-yl)methyl)amino)-N-(3-((6-(3-cyano-1H-pyrazol-4-yl)-1H-indazol-4-yl)amino)propyl)propanamide;
3-(((2-chloro-[1,1′-biphenyl]-4-yl)methyl)amino)-N-(3-((6-(pyridin-4-yl)-1H-benzo[d][1,2,3]triazol-4-yl)amino)propyl)propanamide;
3-(((2-chloro-[1,1′-biphenyl]-4-yl)methyl)amino)-N-(3-((6-(2-methyl-1H-imidazol-4-yl)-1H-indazol-4-yl)amino)propyl)propanamide;
3-(((2-chloro-[1,1′-biphenyl]-4-yl)methyl)amino)-N-(3-((6-cyano-1H-indazol-4-yl)amino)propyl)propanamide;
N-(3-((6-(4H-1,2,4-triazol-4-yl)-1H-indazol-4-yl)amino)propyl)-3-(((2-chloro-[1,1′-biphenyl]-4-yl)methyl)amino)propanamide;
N-(6-(4H-1,2,4-triazol-4-yl)-1H-indazol-4-yl)-3-(3-(((2-chloro-[1,1′-biphenyl]-4-yl)methyl)amino)propanamido)propanamide;
N-(3-((6-(4H-1,2,4-triazol-4-yl)-1H-indazol-4-yl)amino)propyl)-3-((3-chlorobenzyl)amino)propanamide;
N-(2-(4-(2-(((2-chloro-[1,1′-biphenyl]-4-yl)methyl)amino)ethyl)-1H-1,2,3-triazol-1-yl)ethyl)-6-(4H-1,2,4-triazol-4-yl)-1H-indazol-4-amine;
N-(6-(4H-1,2,4-triazol-4-yl)-1H-indazol-4-yl)-2-(2-(2-((3-chloro-4-(trifluoromethoxy)benzyl)amino)ethyl)oxazol-5-yl)acetamide;
N-(2-(5-(2-((3-chloro-4-(trifluoromethoxy)benzyl)amino)ethyl)-4H-1,2,4-triazol-3-yl)ethyl)-6-(4H-1,2,4-triazol-4-yl)-1H-indazol-4-amine;
N-(6-(4H-1,2,4-triazol-4-yl)-1H-indazol-4-yl)-2-((4-((3-chloro-4-(trifluoromethoxy)benzyl)amino)butyl)amino)acetamide;
N-(6-(4H-1,2,4-triazol-4-yl)-1H-indazol-4-yl)-3-(3-((3-chloro-4-(trifluoromethoxy)benzyl)amino)propanamido)propanamide;
N-(3-((6-(4H-1,2,4-triazol-4-yl)-1H-indazol-4-yl)oxy)propyl)-3-((3-chloro-4-(trifluoromethoxy)benzyl)amino)propanamide;
N-(3-((6-(4H-1,2,4-triazol-4-yl)-1H-indazol-4-yl)amino)propyl)-3-((3-chloro-4-(trifluoromethoxy)benzyl)amino)-N-methylpropanamide;
3-(((2-Chloro-[1,1′-biphenyl]-4-yl)methyl)amino)-N-(3-((6-(2-methylpyridin-4-yl)-1H-indazol-4-yl)amino)propyl)propanamide;
3-(((2-Chloro-[1,1′-biphenyl]-4-yl)methyl)amino)-N-(3-((6-(2-methoxypyridin-4-yl)-1H-indazol-4-yl)amino)propyl)propanamide;
N-(2-(4-(((6-chloro-1H-benzo[d]imidazol-2-yl)methyl)amino)butoxy)ethyl)-6-(4H-1,2,4-triazol-4-yl)-1H-indazol-4-amine;
N1-(2-((6-(4H-1,2,4-triazol-4-yl)-1H-indazol-4-yl)amino)ethyl)-N4-(3-chloro-4-(trifluoromethoxy)benzyl)butane-1,4-diamine;
N-(3-((6-(2H-1,2,3-triazol-4-yl)-1H-indazol-4-yl)amino)propyl)-3-(((2-chloro-[1,1′-biphenyl]-4-yl)methyl)amino)propanamide;
3-(((2-chloro-[1,1′-biphenyl]-4-yl)methyl)amino)-N-(3-((6-(pyridin-3-yl)-1H-indazol-4-yl)amino)propyl)propanamide;
3-(((2-chloro-[1,1′-biphenyl]-4-yl)methyl)amino)-N-(3-((6-(3-methyl-1H-pyrazol-4-yl)-1H-indazol-4-yl)amino)propyl)propanamide;
3-(((2-chloro-[1,1′-biphenyl]-4-yl)methyl)amino)-N-(3-((6-(isoxazol-4-yl)-1H-indazol-4-yl)amino)propyl)propanamide;
3-(((2-chloro-[1,1′-biphenyl]-4-yl)methyl)amino)-N-(3-((6-(4-methyl-1H-imidazol-1-yl)-1H-indazol-4-yl)amino)propyl)propanamide;
4-(4-((3-(3-(((2-chloro-[1,1′-biphenyl]-4-yl)methyl)amino)propanamido)propyl)amino)-1H-indazol-6-yl)pyridine 1-oxide;
3-((3-chloro-4-(trifluoromethoxy)benzyl)amino)-N-(3-((6-(2-oxo-1,2-dihydropyridin-4-yl)-1H-indazol-4-yl)amino)propyl)propanamide;
N-(3-((6-(1,2,3-thiadiazol-5-yl)-1H-indazol-4-yl)amino)propyl)-3-((3-chloro-4-(trifluoromethoxy)benzyl)amino)propanamide;
N-(3-((6-(4H-1,2,4-triazol-4-yl)-1H-indazol-4-yl)amino)propyl)-3-((3-chloro-4-(trifluoromethoxy)benzyl)amino)propanamide;
N-(3-((6-(4H-1,2,4-triazol-4-yl)-1H-indazol-4-yl)amino)propyl)-3-(((4-chloro-5-phenyl-1H-pyrazol-3-yl)methyl)amino)propanamide;
N-(3-((6-(4H-1,2,4-triazol-4-yl)-1H-indazol-4-yl)amino)propyl)-3-((3-(hydroxymethyl)benzyl)amino)propanamide;
N-(3-((6-(4H-1,2,4-triazol-4-yl)-1H-indazol-4-yl)amino)propyl)-3-(((6-chloro-1-methyl-1H-indol-2-yl)methyl)amino)propanamide;
N-(3-((6-(4H-1,2,4-triazol-4-yl)-1H-indazol-4-yl)amino)propyl)-3-(((6-chloro-1H-indol-2-yl)methyl)amino)propanamide;
N-(3-((6-(4H-1,2,4-triazol-4-yl)-1H-indazol-4-yl)amino)propyl)-3-(((1-methyl-5-(trifluoromethyl)-1H-indol-2-yl)methyl)amino)propanamide;
N-(3-((6-(4H-1,2,4-triazol-4-yl)-1H-indazol-4-yl)amino)propyl)-3-((3-cyano-4-cyclobutoxybenzyl)amino)propanamide;
N-(3-((6-(4H-1,2,4-triazol-4-yl)-1H-indazol-4-yl)amino)propyl)-3-((3-(2-hydroxyethyl)benzyl)amino)propanamide;
N-(3-((6-(4H-1,2,4-triazol-4-yl)-1H-indazol-4-yl)amino)propyl)-3-((4-cyclobutoxy-3-(hydroxymethyl)benzyl)amino)propanamide;
N-(3-((6-(4H-1,2,4-triazol-4-yl)-1H-indazol-4-yl)amino)propyl)-3-((3-(2-hydroxyethoxy)benzyl)amino)propanamide;
N-(3-((6-(4H-1,2,4-triazol-4-yl)-1H-indazol-4-yl)amino)propyl)-3-((3-chloro-5-(hydroxymethyl)benzyl)amino)propanamide;
N-(3-((6-(4H-1,2,4-triazol-4-yl)-1H-indazol-4-yl)amino)propyl)-3-((3-(hydroxymethyl)-4-(trifluoromethoxy)benzyl)amino)propanamide;
N-(3-((6-(4H-1,2,4-triazol-4-yl)-1H-indazol-4-yl)amino)propyl)-3-((3-cyano-4-cyclopropylbenzyl)amino)propanamide;
N-(3-((6-(4H-1,2,4-triazol-4-yl)-1H-indazol-4-yl)amino)propyl)-3-((3-cyano-4-(trifluoromethoxy)benzyl)amino)propanamide;
N-(3-((6-(4H-1,2,4-triazol-4-yl)-1H-indazol-4-yl)amino)propyl)-3-((3-(cyanomethyl)benzyl)amino)propanamide;
N-(6-(4H-1,2,4-triazol-4-yl)-1H-indazol-4-yl)-2-(4-((3-chloro-4-(trifluoromethoxy)benzyl)amino)butoxy)acetamide;
6-(4H-1,2,4-triazol-4-yl)-N-(2-(4-(((5-(trifluoromethyl)-1H-indol-2-yl)methyl)amino)butoxy)ethyl)-1H-indazol-4-amine;
N-(2-(4-(((6-chloro-1-methyl-1H-indol-2-yl)methyl)amino)butoxy)ethyl)-6-(4H-1,2,4-triazol-4-yl)-1H-indazol-4-amine;
N-(2-(4-(((1-methyl-5-(trifluoromethyl)-1H-indol-2-yl)methyl)amino)butoxy)ethyl)-6-(4H-1,2,4-triazol-4-yl)-1H-indazol-4-amine;
N-(2-(4-(((6-chloro-1H-indol-2-yl)methyl)amino)butoxy)ethyl)-6-(4H-1,2,4-triazol-4-yl)-1H-indazol-4-amine;
N-(2-(4-(((5-cyclobutoxy-1H-indol-2-yl)methyl)amino)butoxy)ethyl)-6-(4H-1,2,4-triazol-4-yl)-1H-indazol-4-amine;
N-((5-cyclobutoxy-1H-indol-2-yl)methyl)-4-(2-((6-(isoxazol-4-yl)-1H-indazol-4-yl)oxy)ethoxy)butan-1-amine;
N-((5-cyclobutoxy-1H-indol-2-yl)methyl)-4-(2-((6-(pyridazin-4-yl)-1H-indazol-4-yl)oxy)ethoxy)butan-1-amine;
4-(2-((6-(pyridazin-4-yl)-1H-indazol-4-yl)oxy)ethoxy)-N-((5-(trifluoromethyl)-1H-indol-2-yl)methyl)butan-1-amine;
N-(2-(4-(((5-cyclobutoxy-1H-indol-2-yl)methyl)amino)butoxy)ethyl)-6-(pyridazin-4-yl)-1H-indazol-4-amine;
N-(3-((6-(4H-1,2,4-triazol-4-yl)-1H-indazol-4-yl)amino)propyl)-3-(((2-chloro-2′-(hydroxymethyl)-[1,1′-biphenyl]-4-yl)methyl)amino)propanamide;
4-((3-(3-(((2-chloro-[1,1′-biphenyl]-4-yl)methyl)amino)propanamido)propyl)amino)-1H-indazole-6-carboxylic acid;
4-((4-((4-(((2-chloro-[1,1′-biphenyl]-4-yl)methyl)amino)butyl)amino)butyl)amino)-1H-indazole-6-carboxylic acid;
N-(3-((1H-indazol-4-yl)amino)propyl)-3-(((2-chloro-[1,1′-biphenyl]-4-yl)methyl)amino)propanamide;
4-((3-(3-(((2-chloro-[1,1′-biphenyl]-4-yl)methyl)amino)propanamido)propyl)amino)-N,N-dimethyl-1H-indazole-6-carboxamide;
3-(((2-chloro-[1,1′-biphenyl]-4-yl)methyl)amino)-N-(3-((6-(hydroxymethyl)-1H-indazol-4-yl)amino)propyl)propanamide;
N-(2-(2-(2-((3-chloro-4-(trifluoromethoxy)benzyl)amino)ethyl)oxazol-5-yl)ethyl)-6-(4H-1,2,4-triazol-4-yl)-1H-indazol-4-amine;
N-(6-(4H-1,2,4-triazol-4-yl)-1H-indazol-4-yl)-2-(5-(2-((3-chloro-4-(trifluoromethoxy)benzyl)amino)ethyl)-4H-1,2,4-triazol-3-yl)acetamide;
N-(6-(4H-1,2,4-triazol-4-yl)-1H-indazol-4-yl)-3-((3-((3-chloro-4-(trifluoromethoxy)benzyl)amino)propyl)amino)propanamide;
N1-(3-((6-(4H-1,2,4-triazol-4-yl)-1H-indazol-4-yl)oxy)propyl)-N3-(3-chloro-4-(trifluoromethoxy)benzyl)propane-1,3-diamine;
N-(2-(4-(((6-chloro-1-methyl-1H-benzo[d]imidazol-2-yl)methyl)amino)butoxy)ethyl)-6-(4H-1,2,4-triazol-4-yl)-1H-indazol-4-amine;
3-(3-((6-(4H-1,2,4-triazol-4-yl)-1H-indazol-4-yl)oxy)propoxy)-N-(3-chloro-4-(trifluoromethoxy)benzyl)propan-1-amine;
N-(6-(4H-1,2,4-triazol-4-yl)-1H-indazol-4-yl)-3-(3-((3-chloro-4-(trifluoromethoxy)benzyl)amino)propoxy)propanamide;
3-(((2-chloro-[1,1′-biphenyl]-4-yl)methyl)amino)-N-(3-((6-(3-methyl-4H-1,2,4-triazol-4-yl)-1H-indazol-4-yl)amino)propyl)propanamide;
N1-(2-((6-(4H-1,2,4-triazol-4-yl)-1H-indazol-4-yl)amino)ethyl)-N4-(3-chloro-4-(trifluoromethoxy)benzyl)-N1-methylbutane-1,4-diamine;
3-(((2-chloro-[1,1′-biphenyl]-4-yl)methyl)amino)-N-(3-((6-(pyrimidin-5-yl)-1H-indazol-4-yl)amino)propyl)propanamide;
3-(((2-chloro-[1,1′-biphenyl]-4-yl)methyl)amino)-N-(3-((6-(2-cyanopyridin-4-yl)-1H-indazol-4-yl)amino)propyl)propanamide;
3-(((2-chloro-[1,1′-biphenyl]-4-yl)methyl)amino)-N-(3-((6-(pyrimidin-4-yl)-1H-indazol-4-yl)amino)propyl)propanamide;
3-(((2-chloro-[1,1′-biphenyl]-4-yl)methyl)amino)-N-(3-((6-(3-methoxy-1H-pyrazol-4-yl)-1H-indazol-4-yl)amino)propyl)propanamide;
N-(3-((6-(4H-1,2,4-triazol-4-yl)-1H-indazol-4-yl)amino)propyl)-3-((4-cyclopropyl-3-(hydroxymethyl)benzyl)amino)propanamide;
3-(((2-chloro-[1,1′-biphenyl]-4-yl)methyl)amino)-N-(3-((6-morpholino-1H-indazol-4-yl)amino)propyl)propanamide;
N-(3-((6-(2H-tetrazol-5-yl)-1H-indazol-4-yl)amino)propyl)-3-(((2-chloro-[1,1′-biphenyl]-4-yl)methyl)amino)propanamide;
3-(((2-chloro-[1,1′-biphenyl]-4-yl)methyl)amino)-N-(3-((6-(2-methyl-1H-imidazol-1-yl)-1H-indazol-4-yl)amino)propyl)propanamide;
3-(((2-chloro-[1,1′-biphenyl]-4-yl)methyl)amino)-N-(3-((6-(oxetan-3-yl)-1H-indazol-4-yl)amino)propyl)propanamide;
3-(((2-chloro-[1,1′-biphenyl]-4-yl)methyl)amino)-N-(3-((6-(tetrahydrofuran-3-yl)-1H-indazol-4-yl)amino)propyl)propanamide;
N-(3-((5-(4H-1,2,4-triazol-4-yl)-1H-indazol-7-yl)amino)propyl)-3-((3-chloro-4-(trifluoromethoxy)benzyl)amino)propanamide;
methyl 4-((2-(4-(((5-(trifluoromethyl)-1H-indol-2-yl)methyl)amino)butoxy)ethyl)amino)-1H-indazole-6-carboxylate;
4-((2-(4-(((5-(trifluoromethyl)-1H-indol-2-yl)methyl)amino)butoxy)ethyl)amino)-1H-indazole-6-carboxylic acid;
6-(3-methyl-4H-1,2,4-triazol-4-yl)-N-(2-(4-(((5-(trifluoromethyl)-1H-indol-2-yl)methyl)amino)butoxy)ethyl)-1H-indazol-4-amine;
6-(2-methoxypyridin-4-yl)-N-(2-(4-(((5-(trifluoromethyl)-1H-indol-2-yl)methyl)amino)butoxy)ethyl)-1H-indazol-4-amine;
methyl 3-(4-((2-(4-(((5-(trifluoromethyl)-1H-indol-2-yl)methyl)amino)butoxy)ethyl)amino)-1H-indazol-6-yl)propanoate;
3-(4-((2-(4-(((5-(trifluoromethyl)-1H-indol-2-yl)methyl)amino)butoxy)ethyl)amino)-1H-indazol-6-yl)propanoic acid;
3-(((2-chloro-[1,1′-biphenyl]-4-yl)methyl)amino)-N-(3-((6-(tetrahydro-2H-pyran-4-yl)-1H-indazol-4-yl)amino)propyl)propanamide;
3-(((2-chloro-[1,1′-biphenyl]-4-yl)methyl)amino)-N-(3-((6-(4-methyl-1H-imidazol-5-yl)-1H-indazol-4-yl)amino)propyl)propanamide;
6-(3-methoxy-1H-pyrazol-4-yl)-N-(2-(4-(((5-(trifluoromethyl)-1H-indol-2-yl)methyl)amino)butoxy)ethyl)-1H-indazol-4-amine;
6-(pyridazin-4-yl)-N-(2-(4-(((5-(trifluoromethyl)-1H-indol-2-yl)methyl)amino)butoxy)ethyl)-1H-indazol-4-amine;
N-(2-(4-(((5-(trifluoromethyl)-1H-indol-2-yl)methyl)amino)butoxy)ethyl)-1H-indazol-4-amine;
6-(pyrimidin-4-yl)-N-(2-(4-(((5-(trifluoromethyl)-1H-indol-2-yl)methyl)amino)butoxy)ethyl)-1H-indazol-4-amine;
4-((2-(4-(((5-(trifluoromethyl)-1H-indol-2-yl)methyl)amino)butoxy)ethyl)amino)-1H-indazole-6-carbonitrile;
6-(isoxazol-4-yl)-N-(2-(4-(((5-(trifluoromethyl)-1H-indol-2-yl)methyl)amino)butoxy)ethyl)-1H-indazol-4-amine;
N-(2-(4-(((5-cyclobutoxy-1H-indol-2-yl)methyl)amino)butoxy)ethyl)-6-(isoxazol-4-yl)-1H-indazol-4-amine;
N-(2-(4-((3-chloro-4-(trifluoromethoxy)benzyl)amino)butoxy)ethyl)-6-(pyridazin-4-yl)-1H-benzo[d][1,2,3]triazol-4-amine;
6-(pyridazin-4-yl)-N-(2-(4-(((5-(trifluoromethyl)-1H-indol-2-yl)methyl)amino)butoxy)ethyl)-1H-benzo[d][1,2,3]triazol-4-amine;
4-(2-((6-(pyridazin-4-yl)-1H-benzo[d][1,2,3]triazol-4-yl)oxy)ethoxy)-N-((5-(trifluoromethyl)-1H-indol-2-yl)methyl)butan-1-amine;
N-(3-chloro-4-(trifluoromethoxy)benzyl)-4-(2-((6-(pyridazin-4-yl)-1H-benzo[d][1,2,3]triazol-4-yl)oxy)ethoxy)butan-1-amine;
2-(3-(((4-((1-(6-(4H-1,2,4-triazol-4-yl)-1H-indazol-4-yl)azetidin-3-yl)oxy)butyl)amino)methyl)-5-fluorophenyl)acetonitrile;
(3-(((4-((1-(6-(4H-1,2,4-triazol-4-yl)-1H-indazol-4-yl)azetidin-3-yl)oxy)butyl)amino)methyl)-5-(trifluoromethyl)phenyl)methanol;
2-(3-(((4-((1-(6-(4H-1,2,4-triazol-4-yl)-1H-indazol-4-yl)azetidin-3-yl)oxy)butyl)amino)methyl)-5-(trifluoromethyl)phenyl)acetonitrile;
2-(3-(((4-((1-(6-(4H-1,2,4-triazol-4-yl)-1H-indazol-4-yl)azetidin-3-yl)oxy)butyl)amino)methyl)-5-methylphenyl)acetonitrile;
N-(3-chloro-4-(trifluoromethoxy)benzyl)-4-((1-(6-(pyridazin-4-yl)-1H-indazol-4-yl)azetidin-3-yl)oxy)butan-1-amine;
N-(3-fluoro-4-(trifluoromethoxy)benzyl)-4-((1-(6-(pyridazin-4-yl)-1H-indazol-4-yl)azetidin-3-yl)oxy)butan-1-amine;
N-((5-cyclobutoxy-1H-indol-2-yl)methyl)-4-((1-(6-(pyridazin-4-yl)-1H-indazol-4-yl)azetidin-3-yl)oxy)butan-1-amine;
(3-fluoro-5-(((4-((1-(6-(pyridazin-4-yl)-1H-indazol-4-yl)azetidin-3-yl)oxy)butyl)amino)methyl)phenyl)methanol;
(3-chloro-5-(((4-((1-(6-(pyridazin-4-yl)-1H-indazol-4-yl)azetidin-3-yl)oxy)butyl)amino)methyl)phenyl)methanol;
(3-(((4-((1-(6-(pyridazin-4-yl)-1H-indazol-4-yl)azetidin-3-yl)oxy)butyl)amino)methyl)-5-(trifluoromethyl)phenyl)methanol;
2-(3-fluoro-5-(((4-((1-(6-(pyridazin-4-yl)-1H-indazol-4-yl)azetidin-3-yl)oxy)butyl)amino)methyl)phenyl)acetonitrile;
2-(3-(((4-((1-(6-(pyridazin-4-yl)-1H-indazol-4-yl)azetidin-3-yl)oxy)butyl)amino)methyl)-5-(trifluoromethyl)phenyl)acetonitrile;
2-(3-methyl-5-(((4-((1-(6-(pyridazin-4-yl)-1H-indazol-4-yl)azetidin-3-yl)oxy)butyl)amino)methyl)phenyl)acetonitrile;
4-(2-((6-(4H-1,2,4-triazol-4-yl)-1H-benzo[d][1,2,3]triazol-4-yl)oxy)ethoxy)-N-((5-(trifluoromethyl)-1H-indol-2-yl)methyl)butan-1-amine;
4-(2-((6-(4H-1,2,4-triazol-4-yl)-1H-indazol-4-yl)oxy)ethoxy)-N-((5-(trifluoromethyl)-1H-indol-2-yl)methyl)butan-1-amine;
4-(2-((6-(4H-1,2,4-triazol-4-yl)-1H-indazol-4-yl)oxy)ethoxy)-N-((5-cyclobutoxy-1H-indol-2-yl)methyl)butan-1-amine;
N-((5-cyclobutoxy-1H-indol-2-yl)methyl)-4-((1-(6-(isoxazol-4-yl)-1H-indazol-4-yl)azetidin-3-yl)oxy)butan-1-amine;
(3-(((4-((1-(6-(isoxazol-4-yl)-1H-indazol-4-yl)azetidin-3-yl)oxy)butyl)amino)methyl)-5-(trifluoromethyl)phenyl)methanol;
2-(3-(((4-((1-(6-(isoxazol-4-yl)-1H-indazol-4-yl)azetidin-3-yl)oxy)butyl)amino)methyl)-5-methylphenyl)acetonitrile;
2-(3-chloro-5-(((4-((1-(6-(isoxazol-4-yl)-1H-indazol-4-yl)azetidin-3-yl)oxy)butyl)amino)methyl)phenyl)acetonitrile;
4-(2-((6-(4H-1,2,4-triazol-4-yl)-1H-benzo[d][1,2,3]triazol-4-yl)oxy)ethoxy)-N-(3-chloro-4-(trifluoromethoxy)benzyl)butan-1-amine;
4-((1-(6-(4H-1,2,4-triazol-4-yl)-1H-indazol-4-yl)azetidin-3-yl)oxy)-N-((5-cyclobutoxy-1H-indol-2-yl)methyl)butan-1-amine:
(3-(((4-((1-(6-(4H-1,2,4-triazol-4-yl)-1H-indazol-4-yl)azetidin-3-yl)oxy)butyl)amino)methyl)-5-fluorophenyl)methanol;
N-(2-(4-((3-chloro-4-(trifluoromethoxy)benzyl)amino)butoxy)ethyl)-6-(4H-1,2,4-triazol-4-yl)-1H-benzo[d][1,2,3]triazol-4-amine;
N-(2-(4-((3-chloro-4-(trifluoromethoxy)benzyl)amino)butoxy)ethyl)-6-(isoxazol-4-yl)-1H-benzo[d][1,2,3]triazol-4-amine;
4-((1-(6-(4H-1,2,4-triazol-4-yl)-1H-indazol-4-yl)azetidin-3-yl)oxy)-N-((5-(trifluoromethyl)-1H-indol-2-yl)methyl)butan-1-amine;
4-((1-(6-(4H-1,2,4-triazol-4-yl)-1H-indazol-4-yl)azetidin-3-yl)oxy)-N-(3-fluoro-4-(trifluoromethoxy)benzyl)butan-1-amine;
(3-(((4-((1-(6-(4H-1,2,4-triazol-4-yl)-1H-indazol-4-yl)azetidin-3-yl)oxy)butyl)amino)methyl)-5-chlorophenyl)methanol;
2-(3-(((4-((1-(6-(4H-1,2,4-triazol-4-yl)-1H-indazol-4-yl)azetidin-3-yl)oxy)butyl)amino)methyl)-5-chlorophenyl)acetonitrile;
4-((1-(6-(4H-1,2,4-triazol-4-yl)-1H-indazol-4-yl)azetidin-3-yl)oxy)-N-(3-chloro-4-(trifluoromethoxy)benzyl)butan-1-amine;
2-(3-chloro-5-(((4-((1-(6-(pyridazin-4-yl)-1H-indazol-4-yl)azetidin-3-yl)oxy)butyl)amino)methyl)phenyl)acetonitrile;
2-(3-(((4-((1-(6-(pyridazin-4-yl)-1H-indazol-4-yl)azetidin-3-yl)oxy)butyl)amino)methyl)-5-(trifluoromethoxy)phenyl)acetonitrile;
N-(3-fluoro-4-(trifluoromethoxy)benzyl)-4-((1-(6-(isoxazol-4-yl)-1H-indazol-4-yl)azetidin-3-yl)oxy)butan-1-amine;
(3-chloro-5-(((4-((1-(6-(isoxazol-4-yl)-1H-indazol-4-yl)azetidin-3-yl)oxy)butyl)amino)methyl)phenyl)methanol;
N-(3-chloro-4-(trifluoromethoxy)benzyl)-4-((1-(6-(isoxazol-4-yl)-1H-indazol-4-yl)azetidin-3-yl)oxy)butan-1-amine;
(3-fluoro-5-(((4-((1-(6-(isoxazol-4-yl)-1H-indazol-4-yl)azetidin-3-yl)oxy)butyl)amino)methyl)phenyl)methanol;
4-((1-(6-(isoxazol-4-yl)-1H-indazol-4-yl)azetidin-3-yl)oxy)-N-((5-(trifluoromethyl)-1H-indol-2-yl)methyl)butan-1-amine;
4-((1-(6-(pyridazin-4-yl)-1H-indazol-4-yl)azetidin-3-yl)oxy)-N-((5-(trifluoromethyl)-1H-indol-2-yl)methyl)butan-1-amine;
2-(3-(((4-(2-((6-(4H-1,2,4-triazol-4-yl)-1H-benzo[d][1,2,3]triazol-4-yl)amino)ethoxy)butyl)amino)methyl)-5-(trifluoromethoxy)phenyl)acetonitrile;
6-(4H-1,2,4-triazol-4-yl)-N-(2-(4-(((5-(trifluoromethyl)-1H-indol-2-yl)methyl)amino)butoxy)ethyl)-1H-benzo[d][1,2,3]triazol-4-amine:
6-(isoxazol-4-yl)-N-(2-(4-(((5-(trifluoromethyl)-1H-indol-2-yl)methyl)amino)butoxy)ethyl)-1H-benzo[d][1,2,3]triazol-4-amine;
4-(2-((6-(isoxazol-4-yl)-1H-indazol-4-yl)oxy)ethoxy)-N-((5-(trifluoromethyl)-1H-indol-2-yl)methyl)butan-1-amine;
2-(3-(((4-((1-(6-(isoxazol-4-yl)-1H-indazol-4-yl)azetidin-3-yl)oxy)butyl)amino)methyl)-5-(trifluoromethyl)phenyl)acetonitrile;
2-(3-(((4-((1-(6-(isoxazol-4-yl)-1H-indazol-4-yl)azetidin-3-yl)oxy)butyl)amino)methyl)-5-(trifluoromethoxy)phenyl)acetonitrile;
2-(3-(((4-((1-(6-(4H-1,2,4-triazol-4-yl)-1H-indazol-4-yl)azetidin-3-yl)oxy)butyl)amino)methyl)-5-(trifluoromethoxy)phenyl)acetonitrile;
2-(3-(((4-(2-((6-(4H-1,2,4-triazol-4-yl)-1H-benzo[d][1,2,3]triazol-4-yl)oxy)ethoxy)butyl)amino)methyl)-5-(trifluoromethoxy)phenyl)acetonitrile;
4-((1-(6-(pyridazin-4-yl)-1H-benzo[d][1,2,3]triazol-4-yl)azetidin-3-yl)oxy)-N-((5-(trifluoromethyl)-1H-indol-2-yl)methyl)butan-1-amine;
N-(3-chloro-4-(trifluoromethoxy)benzyl)-4-((1-(6-(pyridazin-4-yl)-1H-benzo[d][1,2,3]triazol-4-yl)azetidin-3-yl)oxy)butan-1-amine;
2-(3-(((4-((1-(6-(pyridazin-4-yl)-1H-benzo[d][1,2,3]triazol-4-yl)azetidin-3-yl)oxy)butyl)amino)methyl)-5-(trifluoromethoxy)phenyl)acetonitrile;
4-((1-(6-(4H-1,2,4-triazol-4-yl)-1H-benzo[d][1,2,3]triazol-4-yl)azetidin-3-yl)oxy)-N-((5-(trifluoromethyl)-1H-indol-2-yl)methyl)butan-1-amine;
4-((1-(6-(4H-1,2,4-triazol-4-yl)-1H-benzo[d][1,2,3]triazol-4-yl)azetidin-3-yl)oxy)-N-(3-chloro-4-(trifluoromethoxy)benzyl)butan-1-amine;
2-(3-(((4-((1-(6-(4H-1,2,4-triazol-4-yl)-1H-benzo[d][1,2,3]triazol-4-yl)azetidin-3-yl)oxy)butyl)amino)methyl)-5-(trifluoromethoxy)phenyl)acetonitrile;
2-(3-(((4-(2-((6-(pyridazin-4-yl)-1H-benzo[d][1,2,3]triazol-4-yl)oxy)ethoxy)butyl)amino)methyl)-5-(trifluoromethoxy)phenyl)acetonitrile;
3-((3-chloro-4-(trifluoromethoxy)benzyl)amino)-N-(3-((6-(5-cyano-1H-pyrazol-4-yl)-1H-indazol-4-yl)amino)propyl)propanamide;
N-(3-((6-(1,3,4-oxadiazol-2-yl)-1H-indazol-4-yl)amino)propyl)-3-((3-chloro-4-(trifluoromethoxy)benzyl)amino)propanamide;
3-((3-chloro-4-(trifluoromethoxy)benzyl)amino)-N-(3-((7-chloro-6-(pyridin-4-yl)-1H-indazol-4-yl)amino)propyl)propanamide;
3-((3-chloro-4-(trifluoromethoxy)benzyl)amino)-N-(3-((6-(5-methyl-1H-pyrazol-4-yl)-1H-indazol-4-yl)amino)propyl)propanamide;
3-((3-chloro-4-(trifluoromethoxy)benzyl)amino)-N-(3-((6-cyano-1H-indazol-4-yl)amino)propyl)propanamide;
N-(3-((6-(1H-1,2,3-triazol-1-yl)-1H-indazol-4-yl)amino)propyl)-3-((3-chloro-4-(trifluoromethoxy)benzyl)amino)propanamide;
3-((3-chloro-4-(trifluoromethoxy)benzyl)amino)-N-(3-((6-(3-fluoropyridin-4-yl)-1H-indazol-4-yl)amino)propyl)propanamide;
N-(3-((1H-indazol-4-yl)amino)propyl)-3-((3-chloro-4-(trifluoromethoxy)benzyl)amino)propanamide;
3-((3-chloro-4-(trifluoromethoxy)benzyl)amino)-N-(3-((6-(oxazol-5-yl)-1H-indazol-4-yl)amino)propyl)propanamide;
3-((3-chloro-4-(trifluoromethoxy)benzyl)amino)-N-(3-((6-(3-methylpyridin-4-yl)-1H-indazol-4-yl)amino)propyl)propanamide;
7-chloro-N-(2-(4-((3,5-difluoro-4-(trifluoromethoxy)benzyl)amino)butoxy)ethyl)-6-(pyridin-4-yl)-1H-indazol-4-amine;
4-((2-(4-((3,5-difluoro-4-(trifluoromethoxy)benzyl)amino)butoxy)ethyl)amino)-1H-indazole-6-carbonitrile;
3-((3-chloro-4-(trifluoromethoxy)benzyl)amino)-N-(3-((6-(6-oxo-1,6-dihydropyridazin-4-yl)-1H-indazol-4-yl)amino)propyl)propanamide;
3-((3-chloro-4-(trifluoromethoxy)benzyl)amino)-N-(3-((6-(3-chloropyridin-4-yl)-1H-indazol-4-yl)amino)propyl)propanamide;
3-((3-chloro-4-(trifluoromethoxy)benzyl)amino)-N-(3-((6-(2-oxo-1,2-dihydropyrimidin-4-yl)-1H-indazol-4-yl)amino)propyl)propanamide;
4-(2-(((6-(4H-1,2,4-triazol-4-yl)-1H-indazol-4-yl)methyl)amino)ethoxy)-N-(3,5-difluoro-4-(trifluoromethoxy) benzyl)butan-1-amine;
N-(3-(((6-(4H-1,2,4-triazol-4-yl)-1H-indazol-4-yl)methyl)amino)propyl)-3-((3-chloro-4-(trifluoromethoxy)benzyl)amino)propanamide;
N-(3-((6-(1,2,4-oxadiazol-5-yl)-1H-indazol-4-yl)amino)propyl)-3-((3-chloro-4-(trifluoromethoxy)benzyl)amino)propanamide;
N-(3-((6-(4H-1,2,4-triazol-3-yl)-1H-indazol-4-yl)amino)propyl)-3-((3-chloro-4-(trifluoromethoxy)benzyl)amino)propanamide;
3-((3-chloro-4-(trifluoromethoxy)benzyl)amino)-N-(3-((6-(oxazol-4-yl)-1H-indazol-4-yl)amino)propyl)propanamide;
N-(2-(4-((3,5-difluoro-4-(trifluoromethoxy)benzyl)amino)butoxy)ethyl)-7-fluoro-6-(pyridin-4-yl)-1H-indazol-4-amine;
3-((3-chloro-4-(trifluoromethoxy)benzyl)amino)-N-(3-((6-(3-cyano-4H-1,2,4-triazol-4-yl)-1H-indazol-4-yl)amino)propyl)propanamide;
3-((3-chloro-4-(trifluoromethoxy)benzyl)amino)-N-(3-((7-methyl-6-(pyridin-4-yl)-1H-indazol-4-yl)amino)propyl)propanamide;
N-(2-(4-((3,5-difluoro-4-(trifluoromethoxy)benzyl)amino)butoxy)ethyl)-7-methyl-6-(pyridin-4-yl)-1H-indazol-4-amine;
3-((3-chloro-4-(trifluoromethoxy)benzyl)amino)-N-(3-((7-fluoro-6-(pyridin-4-yl)-1H-indazol-4-yl)amino)propyl)propanamide;
3-((3-chloro-4-(trifluoromethoxy)benzyl)amino)-N-(3-((7-chloro-6-(4H-1,2,4-triazol-4-yl)-1H-indazol-4-yl)amino)propyl)propanamide;
7-chloro-N-(2-(4-((3,5-difluoro-4-(trifluoromethoxy)benzyl)amino)butoxy)ethyl)-6-(4H-1,2,4-triazol-4-yl)-1H-indazol-4-amine;
N-(3-((6-(1,2,4-thiadiazol-5-yl)-1H-indazol-4-yl)amino)propyl)-3-((3-chloro-4-(trifluoromethoxy)benzyl)amino)propanamide;
N-(3-((6-(2-aminopyridin-4-yl)-1H-indazol-4-yl)amino)propyl)-3-((3-chloro-4-(trifluoromethoxy)benzyl)amino)propanamide;
3-((3-chloro-4-(trifluoromethoxy)benzyl)amino)-N-(3-((6-(pyridin-4-yl)-1H-pyrazolo[3,4-b]pyridin-4-yl)amino)propyl)propanamide;
5-chloro-N-(2-(4-((3,5-difluoro-4-(trifluoromethoxy)benzyl)amino)butoxy)ethyl)-6-(4H-1,2,4-triazol-4-yl)-1H-indazol-4-amine;
N-(3-((6-(6-aminopyridazin-4-yl)-1H-indazol-4-yl)amino)propyl)-3-((3-chloro-4-(trifluoromethoxy)benzyl)amino)propanamide;
N-(3-((6-(6-aminopyridazin-4-yl)-1H-indazol-4-yl)amino)propyl)-3-((3-chloro-4-(trifluoromethoxy)benzyl)amino)propanamide;
N-(3-((6-(1,2,4-oxadiazol-3-yl)-1H-indazol-4-yl)amino)propyl)-3-((3-chloro-4-(trifluoromethoxy)benzyl)amino)propanamide;
N4-(2-(4-((3,5-difluoro-4-(trifluoromethoxy) benzyl)amino) butoxy)ethyl)-N3-methyl-6-(4H-1,2,4-triazol-4-yl)-1H-indazole-3,4-diamine;
3-((3-chloro-4-(trifluoromethoxy)benzyl)amino)-N-(3-((3-(methylamino)-6-(4H-1,2,4-triazol-4-yl)-1H-indazol-4-yl)amino)propyl)propanamide;
(S)-(3-(((5-(2-((6-(4H-1,2,4-triazol-4-yl)-1H-indazol-4-yl)amino)ethoxy)pentan-2-yl)amino)methyl)-5-(trifluoromethoxy)phenyl)methanol;
(S)-5-(4-((2-((4-((3-(hydroxymethyl)-5-(trifluoromethoxy)benzyl)amino)pentyl)oxy)ethyl)amino)-1H-indazol-6-yl)pyridazin-3-ol;
(S)-2-(3-(((5-(2-((6-(6-hydroxypyridazin-4-yl)-1H-indazol-4-yl)amino)ethoxy)pentan-2-yl)amino)methyl)-5-(trifluoromethoxy)phenyl)acetonitrile;
(S)-5-(4-((2-((4-((3-(oxazol-5-ylmethyl)-5-(trifluoromethoxy)benzyl)amino)pentyl)oxy)ethyl)amino)-1H-indazol-6-yl)pyridazin-3-ol;
(S)-3-(((5-(2-((6-(isoxazol-4-yl)-1H-indazol-4-yl)amino)ethoxy)pentan-2-yl)amino)methyl)-5-(trifluoromethoxy) benzamide;
4-((2-(4-((3,5-difluoro-4-(trifluoromethoxy)benzyl)amino)butoxy)ethyl)amino)-1H-indazole-6-carboxylic acid;
2-(4-((2-(4-((3,5-difluoro-4-(trifluoromethoxy)benzyl)amino)butoxy)ethyl)amino)-1H-indazol-6-yl)acetic acid;
5-(4-(2-((3-((3-(2-hydroxyethoxy)-5-(trifluoromethoxy)benzyl)amino)cyclobutyl)methoxy)ethoxy)-1H-indazol-6-yl)pyridazin-3-ol;
5-(4-(2-((3-((3-(hydroxymethyl)-5-(trifluoromethoxy)benzyl)amino)cyclobutyl)methoxy)ethoxy)-1H-indazol-6-yl)pyridazin-3-ol;
3-(4-((2-(4-((3,5-difluoro-4-(trifluoromethoxy)benzyl)amino)butoxy)ethyl)amino)-1H-indazol-6-yl)propanoic acid;
2-(3-(((4-(2-((1H-indazol-4-yl)amino)ethoxy)butyl)amino)methyl)-5-(trifluoromethoxy)phenoxy)ethanol;
(3-(((3-((2-((6-(1,2,3-thiadiazol-5-yl)-1H-indazol-4-yl)amino)ethoxy)methyl)cyclobutyl)amino)methyl)-5-(trifluoromethoxy)phenyl)methanol;
2-(4-((2-(4-((3-(hydroxymethyl)-5-(trifluoromethoxy)benzyl)amino)butoxy)ethyl)amino)-1H-indazol-6-yl)acetonitrile;
2-(3-(((3-((2-((6-(1,2,3-thiadiazol-5-yl)-1H-indazol-4-yl)amino)ethoxy)methyl)cyclobutyl)amino)methyl)-5-(trifluoromethoxy)phenoxy)ethan-1-ol;
2-(4-((2-(4-((3-(hydroxymethyl)-5-(trifluoromethoxy)benzyl)amino)butoxy)ethyl)amino)-1H-indazol-6-yl)-2-methylpropanenitrile;
(S)-(3-(((5-(2-((6-(1,2,3-thiadiazol-5-yl)-1H-indazol-4-yl)amino)ethoxy)pentan-2-yl)amino)methyl)-5-(trifluoromethoxy)phenyl)methanol; and
(R)-(3-(((4-(2-((6-(1,2,3-thiadiazol-5-yl)-1H-indazol-4-yl)amino)propoxy)butyl)amino)methyl)-5-(trifluoromethoxy)phenyl)methanol.

23. A pharmaceutical composition comprising a compound according to any one of claims 1 to 22, or a pharmaceutically acceptable salt, hydrate or solvate thereof, and a pharmaceutically acceptable excipient.

24. A compound according to any one of claims 1 to 22, or a pharmaceutically acceptable salt of solvate thereof, or a pharmaceutical composition according to claim 23 for use in:
(i) therapy:
(ii) the treatment of a disease or condition in which CK2α activity is implicated;
(iii) the treatment of a disease or condition associated with aberrant activity of CK2α;
(i) the treatment of proliferative disorders (e.g. cancer or benign neoplasms), viral infections, an inflammatory disease or condition, diabetes, vascular and ischemic disorders, neurodegenerative disorders and/or the regulation of circadian rhythm;
(ii) the treatment of a cancer; and/or
(iii) the treatment of a viral infection.
